SEQUENCE LISTING (13 sequences)

SEQ ID NO: 1 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 2 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 3 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 4 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 5 - protein, 467 aa, Homo sapiens [sequence omitted]

SEQ ID NO: 6 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 7 - protein, 22152 aa, Homo sapiens
  MOD_RES (13877)..(13878): Any amino acid
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLXXEXLYWELSXLTXXIXELGPYTLDRXSLYVNGFTHXXSXPTTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMXXXPLLXPFTXNXTITNLXXXXXMXXPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAXXXPLLXPFTXNXTITNLXXXXXMXXPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGATTGMDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLXXEXLYWELSXLTXXIXELGPYTLDRXSLYVNGFTHXXSXPTTSTPGTSTVXXGTSGTPSSXPXXTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTXXXPLLXPFTXNXTITNLXXXXXMXXPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 8 - protein, 89 aa, Homo sapiens
MKGLAAALLVLVCTMALCSCAQVGTNKELCCLVYTSWQIPQKFIVDYSETSPQCPKPGVILLTKRGRQICADPNKKWVQKYISDLKLNA

SEQ ID NO: 9 - protein, 119 aa, Homo sapiens
MAGLMTIVTSLLFLGVCAHHIIPTGSVVIPSPCCMFFVSKRIPENRVVSYQLSSRSTCLKAGVIFTTKKGQQFCGDPKQEWVQRYMKNLDAKQKKASPRARAVAVKGPVQRYPGNQTTC

SEQ ID NO: 10 - protein, 119 aa, Homo sapiens; identical to SEQ ID NO: 9

SEQ ID NO: 11 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 12 - protein, 4 aa, Homo sapiens
ATLG

SEQ ID NO: 13 - protein, 5 aa, Homo sapiens
GCRLY

(19) United States
(12) Patent Application Publication
(10) Pub. No.: US 2013/0165344 A1
(43) Pub. Date: Jun. 27, 2013

(54) METHODS AND COMPOSITIONS FOR DIAGNOSIS AND PROGNOSIS OF RENAL INJURY AND RENAL FAILURE

(71) Applicants: Joseph Anderberg, Encinitas, CA (US); Jeff Gray, Solana Beach, CA (US); Paul McPherson, Encinitas, CA (US); Kevin Nakamura, Cardiff by the Sea, CA (US); James Patrick Kampf, San Diego, CA (US)

(72) Inventors: Joseph Anderberg, Encinitas, CA (US); Jeff Gray, Solana Beach, CA (US); Paul McPherson, Encinitas, CA (US); Kevin Nakamura, Cardiff by the Sea, CA (US); James Patrick Kampf, San Diego, CA (US)

(73) Assignee: ASTUTE MEDICAL, INC., San Diego, CA (US)

(21) Appl. No.: 13/806,759

(22) PCT Filed: Jun. 23, 2011
(86) PCT No.: PCT/US2011/001127

§ 371 Date: Feb. 7, 2013

Related U.S. Application Data
(60) Provisional application No. 61/357,965, filed on Jun. 23, 2010.
(60) Provisional application No. 61/357,956, filed on Jun. 23, 2010.
(60) Provisional application No. 61/364,300, filed on Jul. 14, 2010.
(60) Provisional application No. 61/364,304, filed on Jul. 14, 2010.

(51) Int. Cl. G01N 33/68; G01N 33/74

(52) U.S. Cl. CPC G01N 33/6893; G01N 33/74; G01N 33/6854; G01N 33/6863
USPC 506/9; 436/501; 435/7.4; 435/7.92

(57) ABSTRACT

The present invention relates to methods and compositions for monitoring, diagnosis, prognosis, and determination of treatment regimens in subjects suffering from or suspected of having a renal injury. In particular, the invention relates to using a one or more assays configured to detect a kidney injury marker selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 as diagnostic and prognostic biomarkers in renal injuries.

The present application claims priority to U.S. Provisional Patent Application No. 61/357,965 filed Jun. 23, 2010; U.S. Provisional Patent Application No. 61/357,956 filed Jun. 23, 2010; U.S. Provisional Patent Application No. 61/364,304 filed Jul. 14, 2010; and U.S. Provisional Patent Application No. 61/364,300 filed Jul. 14, 2010, each of which is hereby incorporated in its entirety including all tables, figures, and claims.

### BACKGROUND OF THE INVENTION

The following discussion of the background of the invention is merely provided to aid the reader in understanding the invention and is not admitted to describe or constitute prior art to the present invention.

The kidney is responsible for water and solute excretion from the body. Its functions include maintenance of acid-base balance, regulation of electrolyte concentrations, control of blood volume, and regulation of blood pressure. As such, loss of kidney function through injury and/or disease results in substantial morbidity and mortality. A detailed discussion of renal injuries is provided in Harrison's Principles of Internal Medicine, 17^{th} Ed., a McGraw Hill, New York, pages 1741-1830, which are hereby incorporated by reference in their entirety. Renal disease and/or injury may be acute or chronic. Acute and chronic kidney disease are described as follows (from Current Medical Diagnosis & Treatment 2008, 47^{th} Ed, McGraw Hill, New York, pages 785-815, which are hereby incorporated by reference in their entirety): “Acute renal failure is worsening of renal function over hours to days, resulting in the retention of nitrogenous wastes (such as urea nitrogen) and creatinine in the blood. Retention of these substances is called azotemia. Chronic renal failure (chronic kidney disease) results from an abnormal loss of renal function over months to years”.

Acute renal failure (ARF, also known as acute kidney injury, or AKI) is an abrupt (typically detected within about 48 hours to 1 week) reduction in glomerular filtration. This loss of filtration capacity results in retention of nitrogenous (urea and creatinine) and non-nitrogenous waste products that are normally excreted by the kidney, a reduction in urine output, or both. It is reported that ARF complicates about 5% of hospital admissions, 4-15% of cardiopulmonary bypass surgeries, and up to 30% of intensive care admissions. ARF may be categorized as prerenal, intrinsic renal, or postrenal in causation. Intrinsic renal disease can be further divided into glomerular, tubular, interstitial, and vascular abnormalities. Major causes of ARF are described in the following table, which is adapted from the Merck Manual, 17^{th} ed., Chapter 222, and which is hereby incorporated by reference in their entirety:

| Type | Risk Factors |
|---|---|
| Prerenal | |
| ECF volume depletion | Excessive diuresis, hemorrhage, GI losses, loss of |
| | intravascular fluid into the extravascular space (due to |
| | ascites, peritonitis, pancreatitis, or burns), loss of skin |
| | and mucus membranes, renal salt- and water-wasting states |
| Low cardiac output | Cardiomyopathy, MI, cardiac tamponade, pulmonary embolism, |
| | pulmonary hypertension, positive-pressure mechanical ventilation |
| Low systemic vascular | Septic shock, liver failure, antihypertensive drugs |
| resistance | |
| Increased renal vascular | NSAIDs, cyclosporines, tacrolimus, hypercalcemia, anaphylaxis, |
| resistance | anesthetics, renal artery obstruction, renal vein thrombosis, |
| | sepsis, hepatorenal syndrome |
| Decreased efferent | ACE inhibitors or angiotensin II receptor blockers |
| arteriolar tone (leading to | |
| decreased GFR from | |
| reduced glomerular | |
| transcapillary pressure, | |
| especially in patients with | |
| bilateral renal artery | |
| stenosis) | |
| Intrinsic Renal | |
| Acute tubular injury | Ischemia (prolonged or severe prerenal state): surgery, hemorrhage, |
| | arterial or venous obstruction; Toxins: NSAIDs, cyclosporines, |
| | tacrolimus, aminoglycosides, foscarnet, ethylene glycol, hemoglobin, |
| | myoglobin, ifosfamide, heavy metals, methotrexate, radiopaque contrast |
| | agents, streptozotocin |
| Acute glomerulonephritis | ANCA-associated: Crescentic glomerulonephritis, polyarteritis nodosa, |
| | Wegener's granulomatosis; Anti-GBM glomerulonephritis: |
| | Goodpasture's syndrome; Immune-complex: Lupus glomerulonephritis, |
| | postinfectious glomerulonephritis, cryoglobulinemic glomerulonephritis |
| Acute tubulointerstitial | Drug reaction (eg, β-lactams, NSAIDs, sulfonamides, ciprofloxacin, |
| nephritis | thiazide diuretics, furosemide, phenytoin, allopurinol, pyelonephritis, |
| | papillary necrosis |
| Acute vascular | Vasculitis, malignant hypertension, thrombotic microangiopathies, |
| nephropathy | scleroderma, atheroembolism |
| Infiltrative diseases | Lymphoma, sarcoidosis, leukemia |
| Postrenal | |
| Tubular precipitation | Uric acid (tumor lysis), sulfonamides, triamterene, acyclovir, indinavir, |
| | methotrexate, ethylene glycol ingestion, myeloma protein, myoglobin |
| Ureteral obstruction | Intrinsic: Calculi, clots, sloughed renal tissue, fungus ball, edema, |
| | malignancy, congenital defects; Extrinsic: Malignancy, retroperitoneal |
| | fibrosis, ureteral trauma during surgery or high impact injury |
| Bladder obstruction | Mechanical: Benign prostatic hyperplasia, prostate cancer, bladder cancer, |
| | urethral strictures, phimosis, paraphimosis, urethral valves, obstructed |
| | indwelling urinary catheter; Neurogenic: Anticholinergic drugs, upper or |
| | lower motor neuron lesion |

In the case of ischemic ARF, the course of the disease may be divided into four phases. During an initiation phase, which lasts hours to days, reduced perfusion of the kidney is evolving into injury. Glomerular ultrafiltration reduces, the flow of filtrate is reduced due to debris within the tubules, and back leakage of filtrate through injured epithelium occurs. Renal injury can be mediated during this phase by reperfusion of the kidney. Initiation is followed by an extension phase which is characterized by continued ischemic injury and inflammation and may involve endothelial damage and vascular congestion. During the maintenance phase, lasting from 1 to 2 weeks, renal cell injury occurs, and glomerular filtration and urine output reaches a minimum. A recovery phase can follow in which the renal epithelium is repaired and GFR gradually recovers. Despite this, the survival rate of subjects with ARF may be as low as about 60%.

Acute kidney injury caused by radiocontrast agents (also called contrast media) and other nephrotoxins such as cyclosporine, antibiotics including aminoglycosides and anticancer drugs such as cisplatin manifests over a period of days to about a week. Contrast induced nephropathy (CIN, which is AKI caused by radiocontrast agents) is thought to be caused by intrarenal vasoconstriction (leading to ischemic injury) and from the generation of reactive oxygen species that are directly toxic to renal tubular epithelial cells. CIN classically presents as an acute (onset within 24-48 h) but reversible (peak 3-5 days, resolution within 1 week) rise in blood urea nitrogen and serum creatinine.

A commonly reported criteria for defining and detecting AKI is an abrupt (typically within about 2-7 days or within a period of hospitalization) elevation of serum creatinine. Although the use of serum creatinine elevation to define and detect AKI is well established, the magnitude of the serum creatinine elevation and the time over which it is measured to define AKI varies considerably among publications. Traditionally, relatively large increases in serum creatinine such as 100%, 200%, an increase of at least 100% to a value over 2 mg/dL and other definitions were used to define AKI. However, the recent trend has been towards using smaller serum creatinine rises to define AKI. The relationship between serum creatinine rise, AKI and the associated health risks are reviewed in Praught and Shlipak, Curr Opin Nephrol Hypertens 14:265-270, 2005 and Chertow et al, J Am Soc Nephrol 16: 3365-3370, 2005, which, with the references listed therein, are hereby incorporated by reference in their entirety. As described in these publications, acute worsening renal function (AKI) and increased risk of death and other detrimental outcomes are now known to be associated with very small increases in serum creatinine. These increases may be determined as a relative (percent) value or a nominal value. Relative increases in serum creatinine as small as 20% from the pre-injury value have been reported to indicate acutely worsening renal function (AKI) and increased health risk, but the more commonly reported value to define AKI and increased health risk is a relative increase of at least 25%. Nominal increases as small as 0.3 mg/dL, 0.2 mg/dL or even 0.1 mg/dL have been reported to indicate worsening renal function and increased risk of death. Various time periods for the serum creatinine to rise to these threshold values have been used to define AKI, for example, ranging from 2 days, 3 days, 7 days, or a variable period defined as the time the patient is in the hospital or intensive care unit. These studies indicate there is not a particular threshold serum creatinine rise (or time period for the rise) for worsening renal function or AKI, but rather a continuous increase in risk with increasing magnitude of serum creatinine rise.

One study (Lassnigg et all, J Am Soc Nephrol 15:1597-1605, 2004, hereby incorporated by reference in its entirety) investigated both increases and decreases in serum creatinine. Patients with a mild fall in serum creatinine of −0.1 to −0.3 mg/dL following heart surgery had the lowest mortality rate. Patients with a larger fall in serum creatinine (more than or equal to −0.4 mg/dL) or any increase in serum creatinine had a larger mortality rate. These findings caused the authors to conclude that even very subtle changes in renal function (as detected by small creatinine changes within 48 hours of surgery) seriously effect patient's outcomes. In an effort to reach consensus on a unified classification system for using serum creatinine to define AKI in clinical trials and in clinical practice, Bellomo et al., Crit. Care. 8(4):R204-12, 2004, which is hereby incorporated by reference in its entirety, proposes the following classifications for stratifying AKI patients:

“Risk”: serum creatinine increased 1.5 fold from baseline OR urine production of <0.5 ml/kg body weight/hr for 6 hours;
“Injury”: serum creatinine increased 2.0 fold from baseline OR urine production <0.5 ml/kg/hr for 12 h;
“Failure”: serum creatinine increased 3.0 fold from baseline OR creatinine >355 μmol/l (with a rise of >44) or urine output below 0.3 ml/kg/hr for 24 h or anuria for at least 12 hours;
And included two clinical outcomes:
“Loss”: persistent need for renal replacement therapy for more than four weeks.
“ESRD”: end stage renal disease—the need for dialysis for more than 3 months.

These criteria are called the RIFLE criteria, which provide a useful clinical tool to classify renal status. As discussed in Kellum, Crit. Care Med. 36: S141-45, 2008 and Ricci et al., Kidney Int. 73, 538-546, 2008, each hereby incorporated by reference in its entirety, the RIFLE criteria provide a uniform definition of AKI which has been validated in numerous studies.

More recently, Mehta et al., Crit. Care 11:R31 (doi:10.1186.cc5713), 2007, hereby incorporated by reference in its entirety, proposes the following similar classifications for stratifying AKI patients, which have been modified from RIFLE:
“Stage I”: increase in serum creatinine of more than or equal to 0.3 mg/dL (≧26.4 mol/L) or increase to more than or equal to 150% (1.5-fold) from baseline OR urine output less than 0.5 mL/kg per hour for more than 6 hours;
“Stage II”: increase in serum creatinine to more than 200% (>2-fold) from baseline OR urine output less than 0.5 mL/kg per hour for more than 12 hours;

“Stage III”: increase in serum creatinine to more than 300% (>3-fold) from baseline OR serum creatinine ≧354 μmol/L accompanied by an acute increase of at least 44 μmol/L OR urine output less than 0.3 mL/kg per hour for 24 hours or anuria for 12 hours.

The CIN Consensus Working Panel (McCollough et al, Rev Cardiovasc Med. 2006; 7(4):177-197, hereby incorporated by reference in its entirety) uses a serum creatinine rise of 25% to define Contrast induced nephropathy (which is a type of AKI). Although various groups propose slightly different criteria for using serum creatinine to detect AKI, the consensus is that small changes in serum creatinine, such as 0.3 mg/dL or 25%, are sufficient to detect AKI (worsening renal function) and that the magnitude of the serum creatinine change is an indicator of the severity of the AKI and mortality risk.

Although serial measurement of serum creatinine over a period of days is an accepted method of detecting and diagnosing AKI and is considered one of the most important tools to evaluate AKI patients, serum creatinine is generally regarded to have several limitations in the diagnosis, assessment and monitoring of AKI patients. The time period for serum creatinine to rise to values (e.g., a 0.3 mg/dL or 25% rise) considered diagnostic for AKI can be 48 hours or longer depending on the definition used. Since cellular injury in AKI can occur over a period of hours, serum creatinine elevations detected at 48 hours or longer can be a late indicator of injury, and relying on serum creatinine can thus delay diagnosis of AKI. Furthermore, serum creatinine is not a good indicator of the exact kidney status and treatment needs during the most acute phases of AKI when kidney function is changing rapidly. Some patients with AKI will recover fully, some will need dialysis (either short term or long term) and some will have other detrimental outcomes including death, major adverse cardiac events and chronic kidney disease. Because serum creatinine is a marker of filtration rate, it does not differentiate between the causes of AKI (pre-renal, intrinsic renal, post-renal obstruction, atheroembolic, etc) or the category or location of injury in intrinsic renal disease (for example, tubular, glomerular or interstitial in origin). Urine output is similarly limited, Knowing these things can be of vital importance in managing and treating patients with AKI.

These limitations underscore the need for better methods to detect and assess AKI, particularly in the early and subclinical stages, but also in later stages when recovery and repair of the kidney can occur. Furthermore, there is a need to better identify patients who are at risk of having an AKI.

### BRIEF SUMMARY OF THE INVENTION

It is an object of the invention to provide methods and compositions for evaluating renal function in a subject. As described herein, measurement of one or more biomarkers selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 (each referred to herein as a “kidney injury marker”) can be used for diagnosis, prognosis, risk stratification, staging, monitoring, categorizing and determination of further diagnosis and treatment regimens in subjects suffering or at risk of suffering from an injury to renal function, reduced renal function, and/or acute renal failure (also called acute kidney injury).

The kidney injury markers of the present invention may be used, individually or in panels comprising a plurality of kidney injury markers, for risk stratification (that is, to identify subjects at risk for a future injury to renal function, for future progression to reduced renal function, for future progression to ARF, for future improvement in renal function, etc.); for diagnosis of existing disease (that is, to identify subjects who have suffered an injury to renal function, who have progressed to reduced renal function, who have progressed to ARF, etc.); for monitoring for deterioration or improvement of renal function; and for predicting a future medical outcome, such as improved or worsening renal function, a decreased or increased mortality risk, a decreased or increased risk that a subject will require renal replacement therapy (i.e., hemodialysis, peritoneal dialysis, hemofiltration, and/or renal transplantation, a decreased or increased risk that a subject will recover from an injury to renal function, a decreased or increased risk that a subject will recover from ARF, a decreased or increased risk that a subject will progress to end stage renal disease, a decreased or increased risk that a subject will progress to chronic renal failure, a decreased or increased risk that a subject will suffer rejection of a transplanted kidney, etc.

In a first aspect, the present invention relates to methods for evaluating renal status in a subject. These methods comprise performing an assay method that is configured to detect one or more biomarkers selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 is/are then correlated to the renal status of the subject. This correlation to renal status may include correlating the assay result(s) to one or more of risk stratification, diagnosis, prognosis, staging, classifying and monitoring of the subject as described herein. Thus, the present invention utilizes one or more kidney injury markers of the present invention for the evaluation of renal injury.

In certain embodiments, the methods for evaluating renal status described herein are methods for risk stratification of the subject; that is, assigning a likelihood of one or more future changes in renal status to the subject. In these embodiments, the assay result(s) is/are correlated to one or more such future changes. The following are preferred risk stratification embodiments.

In preferred risk stratification embodiments, these methods comprise determining a subject's risk for a future injury to renal function, and the assay result(s) is/are correlated to a likelihood of such a future injury to renal function. For example, the measured concentration(s) may each be compared to a threshold value. For a “positive going” kidney injury marker, an increased likelihood of suffering a future injury to renal function is assigned to the subject when the measured concentration is above the threshold, relative to a likelihood assigned when the measured concentration is below the threshold. For a “negative going” kidney injury marker, an increased likelihood of suffering a future injury to renal function is assigned to the subject when the measured concentration is below the threshold, relative to a likelihood assigned when the measured concentration is above the threshold.

In other preferred risk stratification embodiments, these methods comprise determining a subject's risk for future reduced renal function, and the assay result(s) is/are correlated to a likelihood of such reduced renal function. For example, the measured concentrations may each be compared to a threshold value. For a “positive going” kidney injury marker, an increased likelihood of suffering a future reduced renal function is assigned to the subject when the measured concentration is above the threshold, relative to a likelihood assigned when the measured concentration is below the threshold. For a “negative going” kidney injury marker, an increased likelihood of future reduced renal function is assigned to the subject when the measured concentration is below the threshold, relative to a likelihood assigned when the measured concentration is above the threshold.

In still other preferred risk stratification embodiments, these methods comprise determining a subject's likelihood for a future improvement in renal function, and the assay result(s) is/are correlated to a likelihood of such a future improvement in renal function. For example, the measured concentration(s) may each be compared to a threshold value. For a “positive going” kidney injury marker, an increased likelihood of a future improvement in renal function is assigned to the subject when the measured concentration is below the threshold, relative to a likelihood assigned when the measured concentration is above the threshold. For a “negative going” kidney injury marker, an increased likelihood of a future improvement in renal function is assigned to the subject when the measured concentration is above the threshold, relative to a likelihood assigned when the measured concentration is below the threshold.

In yet other preferred risk stratification embodiments, these methods comprise determining a subject's risk for progression to ARF, and the result(s) is/are correlated to a likelihood of such progression to ARF. For example, the measured concentration(s) may each be compared to a threshold value. For a “positive going” kidney injury marker, an increased likelihood of progression to ARF is assigned to the subject when the measured concentration is above the threshold, relative to a likelihood assigned when the measured concentration is below the threshold. For a “negative going” kidney injury marker, an increased likelihood of progression to ARF is assigned to the subject when the measured concentration is below the threshold, relative to a likelihood assigned when the measured concentration is above the threshold.

And in other preferred risk stratification embodiments, these methods comprise determining a subject's outcome risk, and the assay result(s) is/are correlated to a likelihood of the occurrence of a clinical outcome related to a renal injury suffered by the subject. For example, the measured concentration(s) may each be compared to a threshold value. For a “positive going” kidney injury marker, an increased likelihood of one or more of: acute kidney injury, progression to a worsening stage of AKI, mortality, a requirement for renal replacement therapy, a requirement for withdrawal of renal toxins, end stage renal disease, heart failure, stroke, myocardial infarction, progression to chronic kidney disease, etc., is assigned to the subject when the measured concentration is above the threshold, relative to a likelihood assigned when the measured concentration is below the threshold. For a “negative going” kidney injury marker, an increased likelihood of one or more of: acute kidney injury, progression to a worsening stage of AKI, mortality, a requirement for renal replacement therapy, a requirement for withdrawal of renal toxins, end stage renal disease, heart failure, stroke, myocardial infarction, progression to chronic kidney disease, etc., is assigned to the subject when the measured concentration is below the threshold, relative to a likelihood assigned when the measured concentration is above the threshold.

In such risk stratification embodiments, preferably the likelihood or risk assigned is that an event of interest is more or less likely to occur within 180 days of the time at which the body fluid sample is obtained from the subject. In particularly preferred embodiments, the likelihood or risk assigned relates to an event of interest occurring within a shorter time period such as 18 months, 120 days, 90 days, 60 days, 45 days, 30 days, 21 days, 14 days, 7 days, 5 days, 96 hours, 72 hours, 48 hours, 36 hours, 24 hours, 12 hours, or less. A risk at 0 hours of the time at which the body fluid sample is obtained from the subject is equivalent to diagnosis of a current condition.

In preferred risk stratification embodiments, the subject is selected for risk stratification based on the pre-existence in the subject of one or more known risk factors for prerenal, intrinsic renal, or postrenal ARF. For example, a subject undergoing or having undergone major vascular surgery, coronary artery bypass, or other cardiac surgery; a subject having pre-existing congestive heart failure, preeclampsia, eclampsia, diabetes mellitus, hypertension, coronary artery disease, proteinuria, renal insufficiency, glomerular filtration below the normal range, cirrhosis, serum creatinine above the normal range, or sepsis; or a subject exposed to NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin are all preferred subjects for monitoring risks according to the methods described herein. This list is not meant to be limiting. By “pre-existence” in this context is meant that the risk factor exists at the time the body fluid sample is obtained from the subject. In particularly preferred embodiments, a subject is chosen for risk stratification based on an existing diagnosis of injury to renal function, reduced renal function, or ARF.

In other embodiments, the methods for evaluating renal status described herein are methods for diagnosing a renal injury in the subject; that is, assessing whether or not a subject has suffered from an injury to renal function, reduced renal function, or ARF. In these embodiments, the assay result(s), for example measured concentration(s) of one or more biomarkers selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 is/are correlated to the occurrence or nonoccurrence of a change in renal status. The following are preferred diagnostic embodiments.

In preferred diagnostic embodiments, these methods comprise diagnosing the occurrence or nonoccurrence of an injury to renal function, and the assay result(s) is/are correlated to the occurrence or nonoccurrence of such an injury. For example, each of the measured concentration(s) may be compared to a threshold value. For a positive going marker, an increased likelihood of the occurrence of an injury to renal function is assigned to the subject when the measured concentration is above the threshold (relative to the likelihood assigned when the measured concentration is below the threshold); alternatively, when the measured concentration is below the threshold, an increased likelihood of the nonoccurrence of an injury to renal function may be assigned to the subject (relative to the likelihood assigned when the measured concentration is above the threshold). For a negative going marker, an increased likelihood of the occurrence of an injury to renal function is assigned to the subject when the measured concentration is below the threshold (relative to the likelihood assigned when the measured concentration is above the threshold); alternatively, when the measured concentration is above the threshold, an increased likelihood of the nonoccurrence of an injury to renal function may be assigned to the subject (relative to the likelihood assigned when the measured concentration is below the threshold).

In other preferred diagnostic embodiments, these methods comprise diagnosing the occurrence or nonoccurrence of reduced renal function, and the assay result(s) is/are correlated to the occurrence or nonoccurrence of an injury causing reduced renal function. For example, each of the measured concentration(s) may be compared to a threshold value. For a positive going marker, an increased likelihood of the occurrence of an injury causing reduced renal function is assigned to the subject when the measured concentration is above the threshold (relative to the likelihood assigned when the measured concentration is below the threshold); alternatively, when the measured concentration is below the threshold, an increased likelihood of the nonoccurrence of an injury causing reduced renal function may be assigned to the subject (relative to the likelihood assigned when the measured concentration is above the threshold). For a negative going marker, an increased likelihood of the occurrence of an injury causing reduced renal function is assigned to the subject when the measured concentration is below the threshold (relative to the likelihood assigned when the measured concentration is above the threshold); alternatively, when the measured concentration is above the threshold, an increased likelihood of the nonoccurrence of an injury causing reduced renal function may be assigned to the subject (relative to the likelihood assigned when the measured concentration is below the threshold).

In yet other preferred diagnostic embodiments, these methods comprise diagnosing the occurrence or nonoccurrence of ARF, and the assay result(s) is/are correlated to the occurrence or nonoccurrence of an injury causing ARF. For example, each of the measured concentration(s) may be compared to a threshold value. For a positive going marker, an increased likelihood of the occurrence of ARF is assigned to the subject when the measured concentration is above the threshold (relative to the likelihood assigned when the measured concentration is below the threshold); alternatively, when the measured concentration is below the threshold, an increased likelihood of the nonoccurrence of ARF may be assigned to the subject (relative to the likelihood assigned when the measured concentration is above the threshold). For a negative going marker, an increased likelihood of the occurrence of ARF is assigned to the subject when the measured concentration is below the threshold (relative to the likelihood assigned when the measured concentration is above the threshold); alternatively, when the measured concentration is above the threshold, an increased likelihood of the nonoccurrence of ARF may be assigned to the subject (relative to the likelihood assigned when the measured concentration is below the threshold).

In still other preferred diagnostic embodiments, these methods comprise diagnosing a subject as being in need of renal replacement therapy, and the assay result(s) is/are correlated to a need for renal replacement therapy. For example, each of the measured concentration(s) may be compared to a threshold value. For a positive going marker, an increased likelihood of the occurrence of an injury creating a need for renal replacement therapy is assigned to the subject when the measured concentration is above the threshold (relative to the likelihood assigned when the measured concentration is below the threshold); alternatively, when the measured concentration is below the threshold, an increased likelihood of the nonoccurrence of an injury creating a need for renal replacement therapy may be assigned to the subject (relative to the likelihood assigned when the measured concentration is above the threshold). For a negative going marker, an increased likelihood of the occurrence of an injury creating a need for renal replacement therapy is assigned to the subject when the measured concentration is below the threshold (relative to the likelihood assigned when the measured concentration is above the threshold); alternatively, when the measured concentration is above the threshold, an increased likelihood of the nonoccurrence of an injury creating a need for renal replacement therapy may be assigned to the subject (relative to the likelihood assigned when the measured concentration is below the threshold).

In still other preferred diagnostic embodiments, these methods comprise diagnosing a subject as being in need of renal transplantation, and the assay result(s) is/are correlated to a need for renal transplantation. For example, each of the measured concentration(s) may be compared to a threshold value. For a positive going marker, an increased likelihood of the occurrence of an injury creating a need for renal transplantation is assigned to the subject when the measured concentration is above the threshold (relative to the likelihood assigned when the measured concentration is below the threshold); alternatively, when the measured concentration is below the threshold, an increased likelihood of the nonoccurrence of an injury creating a need for renal transplantation may be assigned to the subject (relative to the likelihood assigned when the measured concentration is above the threshold). For a negative going marker, an increased likelihood of the occurrence of an injury creating a need for renal transplantation is assigned to the subject when the measured concentration is below the threshold (relative to the likelihood assigned when the measured concentration is above the threshold); alternatively, when the measured concentration is above the threshold, an increased likelihood of the nonoccurrence of an injury creating a need for renal transplantation may be assigned to the subject (relative to the likelihood assigned when the measured concentration is below the threshold).

In still other embodiments, the methods for evaluating renal status described herein are methods for monitoring a renal injury in the subject; that is, assessing whether or not renal function is improving or worsening in a subject who has suffered from an injury to renal function, reduced renal function, or ARF. In these embodiments, the assay result(s), for example measured concentration(s) of one or more biomarkers selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 is/are correlated to the occurrence or nonoccurrence of a change in renal status. The following are preferred monitoring embodiments.

In preferred monitoring embodiments, these methods comprise monitoring renal status in a subject suffering from an injury to renal function, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In other preferred monitoring embodiments, these methods comprise monitoring renal status in a subject suffering from reduced renal function, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In yet other preferred monitoring embodiments, these methods comprise monitoring renal status in a subject suffering from acute renal failure, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In other additional preferred monitoring embodiments, these methods comprise monitoring renal status in a subject at risk of an injury to renal function due to the pre-existence of one or more known risk factors for prerenal, intrinsic renal, or postrenal ARF, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In still other embodiments, the methods for evaluating renal status described herein are methods for classifying a renal injury in the subject; that is, determining whether a renal injury in a subject is prerenal, intrinsic renal, or postrenal; and/or further subdividing these classes into subclasses such as acute tubular injury, acute glomerulonephritis acute tubulointerstitial nephritis, acute vascular nephropathy, or infiltrative disease; and/or assigning a likelihood that a subject will progress to a particular RIFLE stage. In these embodiments, the assay result(s), for example measured concentration(s) of one or more biomarkers selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 is/are correlated to a particular class and/or subclass. The following are preferred classification embodiments.

In preferred classification embodiments, these methods comprise determining whether a renal injury in a subject is prerenal, intrinsic renal, or postrenal; and/or further subdividing these classes into subclasses such as acute tubular injury, acute glomerulonephritis acute tubulointerstitial nephritis, acute vascular nephropathy, or infiltrative disease; and/or assigning a likelihood that a subject will progress to a particular RIFLE stage, and the assay result(s) is/are correlated to the injury classification for the subject. For example, the measured concentration may be compared to a threshold value, and when the measured concentration is above the threshold, a particular classification is assigned; alternatively, when the measured concentration is below the threshold, a different classification may be assigned to the subject.

A variety of methods may be used by the skilled artisan to arrive at a desired threshold value for use in these methods. For example, the threshold value may be determined from a population of normal subjects by selecting a concentration representing the 75th, 85th, 90th, 95th, or 99th percentile of a kidney injury marker measured in such normal subjects. Alternatively, the threshold value may be determined from a “diseased” population of subjects, e.g., those suffering from an injury or having a predisposition for an injury (e.g., progression to ARF or some other clinical outcome such as death, dialysis, renal transplantation, etc.), by selecting a concentration representing the 75th, 85th, 90th, 95th, or 99th percentile of a kidney injury marker measured in such subjects. In another alternative, the threshold value may be determined from a prior measurement of a kidney injury marker in the same subject; that is, a temporal change in the level of a kidney injury marker in the subject may be used to assign risk to the subject.

The foregoing discussion is not meant to imply, however, that the kidney injury markers of the present invention must be compared to corresponding individual thresholds. Methods for combining assay results can comprise the use of multivariate logistical regression, loglinear modeling, neural network analysis, n-of-m analysis, decision tree analysis, calculating ratios of markers, etc. This list is not meant to be limiting. In these methods, a composite result which is determined by combining individual markers may be treated as if it is itself a marker; that is, a threshold may be determined for the composite result as described herein for individual markers, and the composite result for an individual patient compared to this threshold.

The ability of a particular test to distinguish two populations can be established using ROC analysis. For example, ROC curves established from a “first” subpopulation which is predisposed to one or more future changes in renal status, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably at least 0.8, even more preferably at least 0.9, and most preferably at least 0.95.

In certain aspects, the measured concentration of one or more kidney injury markers, or a composite of such markers, may be treated as continuous variables. For example, any particular concentration can be converted into a corresponding probability of a future reduction in renal function for the subject, the occurrence of an injury, a classification, etc. In yet another alternative, a threshold that can provide an acceptable level of specificity and sensitivity in separating a population of subjects into “bins” such as a “first” subpopulation (e.g., which is predisposed to one or more future changes in renal status, the occurrence of an injury, a classification, etc.) and a “second” subpopulation which is not so predisposed. A threshold value is selected to separate this first and second population by one or more of the following measures of test accuracy:

an odds ratio greater than 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less;
a specificity of greater than 0.5, preferably at least about 0.6, more preferably at least about 0.7, still more preferably at least about 0.8, even more preferably at least about 0.9 and most preferably at least about 0.95, with a corresponding sensitivity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;
a sensitivity of greater than 0.5, preferably at least about 0.6, more preferably at least about 0.7, still more preferably at least about 0.8, even more preferably at least about 0.9 and most preferably at least about 0.95, with a corresponding specificity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;
at least about 75% sensitivity, combined with at least about 75% specificity;
a positive likelihood ratio (calculated as sensitivity/(1-specificity)) of greater than 1, at least about 2, more preferably at least about 3, still more preferably at least about 5, and most preferably at least about 10; or
a negative likelihood ratio (calculated as (1-sensitivity)/specificity) of less than 1, less than or equal to about 0.5, more preferably less than or equal to about 0.3, and most preferably less than or equal to about 0.1.
The term “about” in the context of any of the above measurements refers to +/−5% of a given measurement.

Multiple thresholds may also be used to assess renal status in a subject. For example, a “first” subpopulation which is predisposed to one or more future changes in renal status, the occurrence of an injury, a classification, etc., and a “second” subpopulation which is not so predisposed can be combined into a single group. This group is then subdivided into three or more equal parts (known as tertiles, quartiles, quintiles, etc., depending on the number of subdivisions). An odds ratio is assigned to subjects based on which subdivision they fall into. If one considers a tertile, the lowest or highest tertile can be used as a reference for comparison of the other subdivisions. This reference subdivision is assigned an odds ratio of 1. The second tertile is assigned an odds ratio that is relative to that first tertile. That is, someone in the second tertile might be 3 times more likely to suffer one or more future changes in renal status in comparison to someone in the first tertile. The third tertile is also assigned an odds ratio that is relative to that first tertile.

In certain embodiments, the assay method is an immunoassay. Antibodies for use in such assays will specifically bind a full length kidney injury marker of interest, and may also bind one or more polypeptides that are “related” thereto, as that term is defined hereinafter. Numerous immunoassay formats are known to those of skill in the art. Preferred body fluid samples are selected from the group consisting of urine, blood, serum, saliva, tears, and plasma. In the case of those kidney injury markers which are membrane proteins as described hereinafter, preferred assays detect soluble forms thereof.

The foregoing method steps should not be interpreted to mean that the kidney injury marker assay result(s) is/are used in isolation in the methods described herein. Rather, additional variables or other clinical indicia may be included in the methods described herein. For example, a risk stratification, diagnostic, classification, monitoring, etc. method may combine the assay result(s) with one or more variables measured for the subject selected from the group consisting of demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score, risk scores of Thakar et al. (J. Am. Soc. Nephrol. 16: 162-68, 2005), Mehran et al. (J. Am. Coll. Cardiol. 44: 1393-99, 2004), Wijeysundera et al. (JAMA 297: 1801-9, 2007), Goldstein and Chawla (Clin. J. Am. Soc. Nephrol. 5: 943-49, 2010), or Chawla et al. (Kidney Intl. 68: 2274-80, 2005)), a glomerular filtration rate, an estimated glomerular filtration rate, a urine production rate, a serum or plasma creatinine concentration, a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatnine ratio, a renal failure index calculated as urine sodium/(urine creatinine/plasma creatinine), a serum or plasma neutrophil gelatinase (NGAL) concentration, a urine NGAL concentration, a serum or plasma cystatin C concentration, a serum or plasma cardiac troponin concentration, a serum or plasma BNP concentration, a serum or plasma NTproBNP concentration, and a serum or plasma proBNP concentration. Other measures of renal function which may be combined with one or more kidney injury marker assay result(s) are described hereinafter and in Harrison's Principles of Internal Medicine, 17^{th} ED., McGraw Hill, New York, pages 1741-1830, and Current Medical Diagnosis & Treatment 2008, 47^{th} Ed, McGraw Hill, New York, pages 785-815, each of which are hereby incorporated by reference in their entirety.

When more than one marker is measured, the individual markers may be measured in samples obtained at the same time, or may be determined from samples obtained at different (e.g., an earlier or later) times. The individual markers may also be measured on the same or different body fluid samples. For example, one kidney injury marker may be measured in a serum or plasma sample and another kidney injury marker may be measured in a urine sample. In addition, assignment of a likelihood may combine an individual kidney injury marker assay result with temporal changes in one or more additional variables.

In various related aspects, the present invention also relates to devices and kits for performing the methods described herein. Suitable kits comprise reagents sufficient for performing an assay for at least one of the described kidney injury markers, together with instructions for performing the described threshold comparisons.

In certain embodiments, reagents for performing such assays are provided in an assay device, and such assay devices may be included in such a kit. Preferred reagents can comprise one or more solid phase antibodies, the solid phase antibody comprising antibody that detects the intended biomarker target(s) bound to a solid support. In the case of sandwich immunoassays, such reagents can also include one or more detectably labeled antibodies, the detectably labeled antibody comprising antibody that detects the intended biomarker target(s) bound to a detectable label. Additional optional elements that may be provided as part of an assay device are described hereinafter.

Detectable labels may include molecules that are themselves detectable (e.g., fluorescent moieties, electrochemical labels, ecl (electrochemical luminescence) labels, metal chelates, colloidal metal particles, etc.) as well as molecules that may be indirectly detected by production of a detectable reaction product (e.g., enzymes such as horseradish peroxidase, alkaline phosphatase, etc.) or through the use of a specific binding molecule which itself may be detectable (e.g., a labeled antibody that binds to the second antibody, biotin, digoxigenin, maltose, oligohistidine, 2,4-dintrobenzene, phenylarsenate, ssDNA, dsDNA, etc.).

Generation of a signal from the signal development element can be performed using various optical, acoustical, and electrochemical methods well known in the art. Examples of detection modes include fluorescence, radiochemical detection, reflectance, absorbance, amperometry, conductance, impedance, interferometry, ellipsometry, etc. In certain of these methods, the solid phase antibody is coupled to a transducer (e.g., a diffraction grating, electrochemical sensor, etc) for generation of a signal, while in others, a signal is generated by a transducer that is spatially separate from the solid phase antibody (e.g., a fluorometer that employs an excitation light source and an optical detector). This list is not meant to be limiting. Antibody-based biosensors may also be employed to determine the presence or amount of analytes that optionally eliminate the need for a labeled molecule.

### DETAILED DESCRIPTION OF THE INVENTION

The present invention relates to methods and compositions for diagnosis, differential diagnosis, risk stratification, monitoring, classifying and determination of treatment regimens in subjects suffering or at risk of suffering from injury to renal function, reduced renal function and/or acute renal failure through measurement of one or more kidney injury markers. In various embodiments, a measured concentration of one or more biomarkers selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 or one or more markers related thereto, are correlated to the renal status of the subject.

For purposes of this document, the following definitions apply:

As used herein, an “injury to renal function” is an abrupt (within 14 days, preferably within 7 days, more preferably within 72 hours, and still more preferably within 48 hours) measurable reduction in a measure of renal function. Such an injury may be identified, for example, by a decrease in glomerular filtration rate or estimated GFR, a reduction in urine output, an increase in serum creatinine, an increase in serum cystatin C, a requirement for renal replacement therapy, etc. “Improvement in Renal Function” is an abrupt (within 14 days, preferably within 7 days, more preferably within 72 hours, and still more preferably within 48 hours) measurable increase in a measure of renal function. Preferred methods for measuring and/or estimating GFR are described hereinafter.

As used herein, “reduced renal function” is an abrupt (within 14 days, preferably within 7 days, more preferably within 72 hours, and still more preferably within 48 hours) reduction in kidney function identified by an absolute increase in serum creatinine of greater than or equal to 0.1 mg/dL (≧8.8 μmol/L), a percentage increase in serum creatinine of greater than or equal to 20% (1.2-fold from baseline), or a reduction in urine output (documented oliguria of less than 0.5 ml/kg per hour).

As used herein, “acute renal failure” or “ARF” is an abrupt (within 14 days, preferably within 7 days, more preferably within 72 hours, and still more preferably within 48 hours) reduction in kidney function identified by an absolute increase in serum creatinine of greater than or equal to 0.3 mg/dI (≧26.4 μmol/L), a percentage increase in serum creatinine of greater than or equal to 50% (1.5-fold from baseline), or a reduction in urine output (documented oliguria of less than 0.5 ml/kg per hour for at least 6 hours). This term is synonymous with “acute kidney injury” or “AKI.”

As used herein, the term “C-C motif chemokine 8” refers to one or more polypeptides present in a biological sample that are derived from the C-C motif chemokine 8 precursor (Swiss-Prot P80075 (SEQ ID NO: 1)).

```
                10 20 30 40 50 60
MKVSAALLCL LLMAATFSPQ GLAQPDSVSI PITCCFNVIN RKIPIQRLES YTRITNIQCP
                70 80 90
KEAVIFKTKR GKEVCADPKE RWVRDSMKHL DQIFQNLKP
```

The following domains have been identified in C-C motif chemokine 8:

| Residues | Length | Domain ID |
|---|---|---|
| 1-23 | 23 | Signal peptide |
| 24-99 | 76 | C-C motif chemokine 8 |
| 29-99 | 76 | MCP-2(6-76) |

As used herein, the term “Interleukin-2 receptor subunit alpha” refers to one or more polypeptides present in a biological sample that are derived from the Interleukin-2 receptor subunit alpha precursor (Swiss-Prot P01589 (SEQ ID NO: 2)):

```
                10 20 30 40 50 60
MDSYLLMWGL LTFIMVPGCQ AELCDDDPPE IPHATFKAMA YKEGTMLNCE CKRGFRRIKS
                70 80 90 100 110 120
GSLYMLCTGN SSHSSWDNQC QCTSSATRNT TKQVTPQPEE QKERKTTEMQ SPMQPVDQAS
              130 140 150 160 170 180
LPGHCREPPP WENEATERIY HFVVGQMVYY QCVQGYRALH RGPAESVCKM THGKTRWTQP
              190 200 210 220 230 240
QLICTGEMET SQFPGEEKPQ ASPEGRPESE TSCLVTTTDF QIQTEMAATM ETSIFTTEYQ
              250 260 270
VAVAGCVFLL ISVLLLSGLT WQRRQRKSRR TI
```

Interleukin-2 receptor subunit alpha is a single-pass type I membrane protein having a large extracellular domain, some or all of which is present, in soluble forms of Interleukin-2 receptor subunit alpha generated either through alternative splicing event which deletes all or a portion of the transmembrane domain, or by proteolysis of the membrane-bound form. In the case of an immunoassay, one or more antibodies that bind to epitopes within this extracellular domain may be used to detect these soluble form(s). The following domains have been identified in Interleukin-2 receptor subunit alpha:

| Residues | Length | Domain ID |
|---|---|---|
| 1-21 | 21 | Signal peptide |
| 22-272 | 251 | Interleukin-2 receptor subunit alpha |
| 22-240 | 219 | Extracellular domain |
| 241-259 | 19 | Transmembrane domain |
| 260-272 | 13 | Cytoplasmic domain |

As used herein, the term “Insulin-like growth factor-binding protein 3” refers to one or more polypeptides present in a biological sample that are derived from the Insulin-like growth factor-binding protein 3 precursor (Swiss-Prot P17936 (SEQ ID NO: 3)).

```
                10 20 30 40 50 60
MQRARPTLWA AALTLLVLLR GPPVARAGAS SAGLGPVVRC EPCDARALAQ CAPPPAVCAE
                70 80 90 100 110 120
LVREPGCGCC LTCALSEGQP CGIYTERCGS GLRCQPSPDE ARPLQALLDG RGLCVNASAV
              130 140 150 160 170 180
SRLRAYLLPA PPAPGNASES EEDRSAGSVE SPSVSSTHRV SDPKFHPLHS KIIIIKKGHA
              190 200 210 220 230 240
KDSQRYKVDY ESQSTDTQNF SSESKRETEY GPCRREMEDT LNHLKFLNVL SPRGVHIPNC
              250 260 270 280 290
DKKGFYKKKQ CRPSKGRKRG FCWCVDKYGQ PLPGYTTKGK EDVHCYSMQS K
```

The following domains have been identified in Insulin-like growth factor-binding protein 3:

| Residues | Length | Domain ID |
|---|---|---|
| 1-27 | 27 | Signal peptide |
| 28-291 | 264 | Insulin-like growth factor-binding protein 3 |

As used herein, the term “Interleukin-11” refers to one or more polypeptides present in a biological sample that are derived from the Interleukin-11 precursor (Swiss-Prot P20809 (SEQ ID NO: 4)).

```
                10 20 30 40 50 60
MNCVCRLVLV VLSLWPDTAV APGPPPGPPR VSPDPRAELD STVLLTRSLL ADTRQLAAQL
                70 80 90 100 110 120
RDKFPADGDH NLDSLPTLAM SAGALGALQL PGVLTRLRAD LLSYLRHVQW LRRAGGSSLK
              130 140 150 160 170 180
TLEPELGTLQ ARLDRLLRRL QLLMSRLALP QPPPDPPAPP LAPPSSAWGG IRAAHAILGG
              190
LHLTLDWAVR GLLLLKTRL
```

The following domains have been identified in Interleukin-11:

| Residues | Length | Domain ID |
|---|---|---|
| 1-21 | 21 | Signal peptide |
| 22-199 | 178 | Interleukin-11 |

As used herein, the term “Neutrophil collagenase” (also known as MMP-8 and matrix metalloproteinase 8) refers to one or more polypeptides present in a biological sample that are derived from the Neutrophil collagenase precursor (Swiss-Prot P22894 (SEQ ID NO: 5)).

```
                10 20 30 40 50 60
MFSLKTLPFL LLLHVQISKA FPVSSKEKNT KTVQDYLEKF YQLPSNQYQS TRKNGTNVIV
                70 80 90 100 110 120
EKLKEMQRFF GLNVTGKPNE ETLDMMKKPR CGVPDSGGFM LTPGNPKWER TNLTYRIRNY
              130 140 150 160 170 180
TPQLSEAEVE RAIKDAFELW SVASPLIFTR ISQGEADINI AFYQRDHGDN SPFDGPNGIL
              190 200 210 220 230 240
AHAFQPGQGI GGDAHFDAEE TWTNTSANYN LFLVAAHEFG HSLGLAHSSD PGALMYPNYA
              250 260 270 280 290 300
FRETSNYSLP QDDIDGIQAI YGLSSNPIQP TGPSTPKPCD PSLTFDAITT LRGEILFFKD
              310 320 330 340 350 360
RYFWRRHPQL QRVEMNFISL FWPSLPTGIQ AAYEDFDRDL IFLFKGNQYW ALSGYDILQG
              370 380 390 400 410 420
YPKDISNYGF PSSVQAIDAA VFYRSKTYFF VNDQFWRYDN QRQFMEPGYP KSISGAFPGI
              430 440 450 460
ESKVDAVFQQ EHFFHVFSGP RYYAFDLIAQ RVTRVARGNK WLNCRYG
```

The following domains have been identified in Neutrophil collagenase:

| Residues | Length | Domain ID |
|---|---|---|
| 1-20 | 20 | Signal peptide |
| 21-100 | 80 | Activation peptide |
| 101-467 | 367 | Neutrophil collagenase |

As used herein, the term “Transforming growth factor alpha” refers to one or more polypeptides present in a biological sample that are derived from the Transforming growth factor alpha precursor (Swiss-Prot P01135 (SEQ ID NO: 6)).

```
                10 20 30 40 50 60
MVPSAGQLAL FALGIVLAAC QALENSTSPL SADPPVAAAV VSHFNDCPDS HTQFCFHGTC
                70 80 90 100 110 120
RFLVQEDKPA CVCHSGYVGA RCEHADLLAV VAASQKKQAI TALVVVSIVA LAVLIITCVL
              130 140 150 160
IHCCQVRKHC EWCRALICRH EKPSALLKGR TACCHSETVV
```

The following domains have been identified in Transforming growth factor alpha:

| Residues | Length | Domain ID |
|---|---|---|
| 1-23 | 23 | Signal peptide |
| 24-160 | 131 | pro-Transforming growth factor alpha |
| 24-39 | 16 | propeptide |
| 40-89 | 50 | Transforming growth factor alpha |
| 90-160 | 71 | propeptide |
| 32 | | Missing in isoform 2 |
| 32 | | Missing in isoform 3 |
| 159-160 | | VV → .ATLG in isoform \dot{3} |
| 159-160 | | VV → GCRLY in isoform 4 |
| 32 | | Missing in isoform 5 |
| 159-160 | | VV → GCRLY in isoform 5 |

As used herein, the term “Cancer Antigen CA 15-3” refers to one or more polypeptides present in a biological sample that are derived from the Mucin-16 precursor (Swiss-Prot Q8WXI7 (SEQ ID NO: 7)):

```
                10 20 30 40 50 60
MLKPSGLPGS SSPTRSLMTG SRSTKATPEM DSGLTGATLS PKTSTGAIVV TEHTLPFTSP
                70 80 90 100 110 120
DKTLASPTSS VVGRTTQSLG VMSSALPEST SRGMTHSEQR TSPSLSPQVN GTPSRNYPAT
              130 140 150 160 170 180
SMVSGLSSPR TRTSSTEGNF TKEASTYTLT VETTSGPVTE KYTVPTETST TEGDSTETPW
              190 200 210 220 230 240
DTRYIPVKIT SPMKTFADST ASKENAPVSM TPAETTVTDS HTPGRTNPSF GTLYSSFLDL
              250 260 270 280 290 300
SPKGTPNSRG ETSLELILST TGYPFSSPEP GSAGHSRIST SAPLSSSASV LDNKISETSI
              310 320 330 340 350 360
FSGQSLTSPL SPGVPEARAS TMPNSAIPFS MTLSNAETSA ERVRSTISSL GTPSISTKQT
              370 380 390 400 410 420
AETILTFHAF AETMDIPSTH IAKTLASEWL GSPGTLGGTS TSALTTTSPS TTLVSEETNT
              430 440 450 460 470 480
HHSTSGKETE GTLNTSMTPL ETSAPGEESE MTATLVPTLG FTTLDSKIRS PSQVSSSHPT
              490 500 510 520 530 540
RELRTTGSTS GRQSSSTAAH GSSDILRATT SSTSKASSWT SESTAQQFSE PQHTQWVETS
              550 560 570 580 590 600
PSMKTERPPA STSVAAPITT SVPSVVSGFT TLKTSSTKGI WLEETSADTL IGESTAGPTT
              610 620 630 640 650 660
HQFAVPTGIS MTGGSSTRGS QGTTHLLTRA TASSETSADL TLATNGVPVS VSPAVSKTAA
              670 680 690 700 710 720
GSSPPGGTKP SYTMVSSVIP ETSSLQSSAF REGTSLGLTP LNTRHPFSSP EPDSAGHTKI
              730 740 750 760 770 780
STSIPLLSSA SVLEDKVSAT STFSHHKATS SITTGTPEIS TKTKPSSAVL SSMTLSNAAT
              790 800 810 820 830 840
SPERVRNATS PLTHPSPSGE ETAGSVLTLS TSAETTDSPN IHPTGTLTSE SSESPSTLSL
              850 860 870 880 890 900
PSVSGVKTTF SSSTPSTHLF TSGEETEETS NPSVSQPETS VSRVRTTLAS TSVPTPVFPT
              910 920 930 940 950 960
MDTWPTRSAQ FSSSHLVSEL RATSSTSVTN STGSALPKIS HLTGTATMSQ TNRDTFNDSA
              970 980 990 1000 1010 1020
APQSTTWPET SPRFKTGLPS ATTTVSTSAT SLSATVMVSK FTSPATSSME ATSIREPSTT
            1030 1040 1050 1060 1070 1080
ILTTETTNGP GSMAVASTNI PIGKGYITEG RLDTSHLPIG TTASSETSMD FTMAKESVSM
            1090 1100 1110 1120 1130 1140
SVSPSQSMDA AGSSTPGRTS QFVDTFSDDV YHLTSREITI PRDGTSSALT PQMTATHPPS
            1150 1160 1170 1180 1190 1200
PDPGSARSTW LGILSSSPSS PTPKVTMSST FSTQRVTTSM IMDTVETSRW NMPNLPSTTS
            1210 1220 1230 1240 1250 1260
LTPSNIPTSG AIGKSTLVPL DTPSPATSLE ASEGGLPTLS TYPESTNTPS IHLGAHASSE
            1270 1280 1290 1300 1310 1320
SPSTIKLTMA SVVKPGSYTP LTFPSIETHI HVSTARMAYS SGSSPEMTAP GETNTGSTWD
            1330 1340 1350 1360 1370 1380
PTTYITTTDP KDTSSAQVST PHSVRTLRTT ENHPKTESAT PAAYSGSPKI SSSPNLTSPA
            1390 1400 1410 1420 1430 1440
TKAWTITDTT EHSTQLHYTK LAEKSSGFET QSAPGPVSVV IPTSPTIGSS TLELTSDVPG
            1450 1460 1470 1480 1490 1500
EPLVLAPSEQ TTITLPMATW LSTSLTEEMA STDLDISSPS SPMSTFAIFP PMSTPSHELS
            1510 1520 1530 1540 1550 1560
KSEADTSAIR NTDSTTLDQH LGIRSLGRTG DLTTVPITPL TTTWTSVIEH STQAQDTLSA
            1570 1580 1590 1600 1610 1620
TMSPTHVTQS LKDQTSIPAS ASPSHLTEVY PELGTQGRSS SEATTFWKPS TDTLSREIET
            1630 1640 1650 1660 1670 1680
GPTNIQSTPP MDNTTTGSSS SGVTLGIAHL PIGTSSPAET STNMALERRS STATVSMAGT
            1690 1700 1710 1720 1730 1740
MGLLVTSAPG RSISQSLGRV SSVLSESTTE GVTDSSKGSS PRLNTQGNTA LSSSLEPSYA
            1750 1760 1770 1780 1790 1800
EGSQMSTSIP LTSSPTTPDV EFIGGSTFWT KEVTTVMTSD ISKSSARTES SSATLMSTAL
            1810 1820 1830 1840 1850 1860
GSTENTGKEK LRTASMDLPS PTPSMEVTPW ISLTLSNAPN TTDSLDLSHG VHTSSAGTLA
            1870 1880 1890 1900 1910 1920
TDRSLNTGVT RASRLENGSD TSSKSLSMGN STHTSMTDTE KSEVSSSIHP RPETSAPGAE
            1930 1940 1950 1960 1970 1980
TTLTSTPGNR AISLTLPFSS IPVEEVISTG ITSGPDINSA PMTHSPITPP TIVWTSTGTI
            1990 2000 2010 2020 2030 2040
EQSTQPLHAV SSEKVSVQTQ STPYVNSVAV SASPTHENSV SSGSSTSSPY SSASLESLDS
            2050 2060 2070 2080 2090 2100
TISRRNAITS WLWDLTTSLP TTTWPSTSLS EALSSGHSGV SNPSSTTTEF PLFSAASTSA
            2110 2120 2130 2140 2150 2160
AKQRNPETET HGPQNTAAST LNTDASSVTG LSETPVGASI SSEVPLPMAI TSRSDVSGLT
            2170 2180 2190 2200 2210 2220
SESTANPSLG TASSAGTKLT RTISLPTSES LVSFRMNKDP WTVSIPLGSH PTTNTETSIP
            2230 2240 2250 2260 2270 2280
VNSAGPPGLS TVASDVIDTP SDGAESIPTV SFSPSPDTEV TTISHFPEKT THSFRTISSL
            2290 2300 2310 2320 2330 2340
THELTSRVTP IPGDWMSSAM STKPTGASPS ITLGERRTIT SAAPTTSPIV LTASFTETST
            2350 2360 2370 2380 2390 2400
VSLDNETTVK TSDILDARKT NELPSDSSSS SDLINTSIAS STMDVTKTAS ISPTSISGMT
            2410 2420 2430 2440 2450 2460
ASSSPSLFSS DRPQVPTSTT ETNTATSPSV SSNTYSLDGG SNVGGTPSTL PPFTITHPVE
            2470 2480 2490 2500 2510 2520
TSSALLAWSR PVRTFSTMVS TDTASGENPT SSNSVVTSVP APGTWASVGS TTDLPAMGFL
            2530 2540 2550 2560 2570 2580
KTSPAGEAHS LLASTIEPAT AFTPHLSAAV VTGSSATSEA SLLTTSESKA IHSSPQTPTT
            2590 2600 2610 2620 2630 2640
PTSGANWETS ATPESLLVVT ETSDTTLTSK ILVTDTILFS TVSTPPSKFP STGTLSGASF
            2650 2660 2670 2680 2690 2700
PTLLPDTPAI PLTATEPTSS LATSFDSTPL VTIASDSLGT VPETTLTMSE TSNGDALVLK
            2710 2720 2730 2740 2750 2760
TVSNPDRSIP GITIQGVTES PLHPSSTSPS KIVAPRNTTY EGSITVALST LPAGTTGSLV
            2770 2780 2790 2800 2810 2820
FSQSSENSET TALVDSSAGL ERASVMPLTT GSQGMASSGG IRSGSTHSTG TKTFSSLPLT
            2830 2840 2850 2860 2870 2880
MNPGEVTAMS EITTNRLTAT QSTAPKGIPV KPTSAESGLL TPVSASSSPS KAFASLTTAP
            2890 2900 2910 2920 2930 2940
PSTWGIPQST LTFEFSEVPS LDTKSASLPT PGQSLNTIPD SDASTASSSL SKSPEKNPRA
            2950 2960 2970 2980 2990 3000
RMMTSTKAIS ASSFQSTGFT ETPEGSASPS MAGHEPRVPT SGTGDPRYAS ESMSYPDPSK
            3010 3020 3030 3040 3050 3060
ASSAMTSTSL ASKLTTLFST GQAARSGSSS SPISLSTEKE TSFLSPTAST SRKTSLFLGP
            3070 3080 3090 3100 3110 3120
SMARQPNILV HLQTSALTLS PTSTLNMSQE EPPELTSSQT IAEEEGTTAE TQTLTFTPSE
            3130 3140 3150 3160 3170 3180
TPTSLLPVSS PTEPTARRKS SPETWASSIS VPAKTSLVET TDGTLVTTIK MSSQAAQGNS
            3190 3200 3210 3220 3230 3240
TWPAPAEETG TSPAGTSPGS PEVSTTLKIM SSKEPSISPE IRSTVRNSPW KTPETTVPME
            3250 3260 3270 3280 3290 3300
TTVEPVTLQS TALGSGSTSI SHLPTGTTSP TKSPTENMLA TERVSLSPSP PEAWTNLYSG
            3310 3320 3330 3340 3350 3360
TPGGTRQSLA TMSSVSLESP TARSITGTGQ QSSPELVSKT TGMEFSMWHG STGGTTGDTH
            3370 3380 3390 3400 3410 3420
VSLSTSSNIL EDPVTSPNSV SSLTDKSKHK TETWVSTTAI PSTVLNNKIM AAEQQTSRSV
            3430 3440 3450 3460 3470 3480
DEAYSSTSSW SDQTSGSDIT LGASPDVTNT LYITSTAQTT SLVSLPSGDQ GITSLTNPSG
            3490 3500 3510 3520 3530 3540
GKTSSASSVT SPSIGLETLR ANVSAVKSDI APTAGHLSQT SSPAEVSILD VTTAPTPGIS
            3550 3560 3570 3580 3590 3600
TTITTMGTNS ISTTTPNPEV GMSTMDSTPA TERRTTSTEH PSTWSSTAAS DSWTVTDMTS
            3610 3620 3630 3640 3650 3660
NLKVARSPGT ISTMHTTSFL ASSTELDSMS TPHGRITVIG TSLVTPSSDA SAVKTETSTS
            3670 3680 3690 3700 3710 3720
ERTLSPSDTT ASTPISTFSR VQRMSISVPD ILSTSWTPSS TEAEDVPVSM VSTDHASTKT
            3730 3740 3750 3760 3770 3780
DPNTPLSTFL FDSLSTLDWD TGRSLSSATA TTSAPQGATT PQELTLETMI SPATSQLPFS
            3790 3800 3810 3820 3830 3840
IGHITSAVTP AAMARSSGVT FSRPDPTSKK AEQTSTQLPT TTSAHPGQVP RSAATTLDVI
            3850 3860 3870 3880 3890 3900
PHTAKTPDAT FQRQGQTALT TEARATSDSW NEKEKSTPSA PWITEMMNSV SEDTIKEVTS
            3910 3920 3930 3940 3950 3960
SSSVLKDPEY AGHKLGIWDD FIPKFGKAAH MRELPLLSPP QDKEAIHPST NTVETTGWVT
            3970 3980 3990 4000 4010 4020
SSEHASHSTI PAHSASSKLT SPVVTTSTRE QAIVSMSTTT WPESTRARTE PNSFLTIELR
            4030 4040 4050 4060 4070 4080
DVSPYMDTSS TTQTSIISSP GSTAITKGPR TEITSSKRIS SSFLAQSMRS SDSPSEAITR
            4090 4100 4110 4120 4130 4140
LSNFPAMTES GGMILAMQTS PPGATSLSAP TLDTSATASW TGTPLATTQR FTYSEKTTLF
            4150 4160 4170 4180 4190 4200
SKGPEDTSQP SPPSVEETSS SSSLVPIHAT TSPSNILLTS QGHSPSSTPP VTSVFLSETS
            4210 4220 4230 4240 4250 4260
GLGKTTDMSR ISLEPGTSLP PNLSSTAGEA LSTYEASRDT KAIHHSADTA VTNMEATSSE
            4270 4280 4290 4300 4310 4320
YSPIPGHTKP SKATSPLVTS HIMGDITSST SVFGSSETTE IETVSSVNQG LQERSTSQVA
            4330 4340 4350 4360 4370 4380
SSATETSTVI THVSSGDATT HVTKTQATFS SGTSISSPHQ FITSTNTFTD VSTNPSTSLI
            4390 4400 4410 4420 4430 4440
MTESSGVTIT TQTGPTGAAT QGPYLLDTST MPYLTETPLA VTPDFMQSEK TTLISKGPKD
            4450 4460 4470 4480 4490 4500
VTWTSPPSVA ETSYPSSLTP FLVTTIPPAT STLQGQHTSS PVSATSVLTS GLVKTTDMLN
            4510 4520 4530 4540 4550 4560
TSMEPVTNSP QNLNNPSNEI LATLAATTDI ETIHPSINKA VTNMGTASSA HVLHSTLPVS
            4570 4580 4590 4600 4610 4620
SEPSTATSPM VPASSMGDAL ASISIPGSET TDIEGEPTSS LTAGRKENST LQEMNSTTES
            4630 4640 4650 4660 4670 4680
NIILSNVSVG AITEATKMEV PSFDATFIPT PAQSTKFPDI FSVASSRLSN SPPMTISTHM
            4690 4700 4710 4720 4730 4740
TTTQTGSSGA TSKIPLALDT STLETSAGTP SVVTEGFAHS KITTAMNNDV KDVSQTNPPF
            4750 4760 4770 4780 4790 4800
QDEASSPSSQ APVLVTTLPS SVAFTPQWHS TSSPVSMSSV LTSSLVKTAG KVDTSLETVT
            4810 4820 4830 4840 4850 4860
SSPQSMSNTL DDISVTSAAT TDIETTHPSI NTVVTNVGTT GSAFESHSTV SAYPEPSKVT
            4870 4880 4890 4900 4910 4920
SPNVTTSTME DTTISRSIPK SSKTTRTETE TTSSLTPKLR ETSISQEITS STETSTVPYK
            4930 4940 4950 4960 4970 4980
ELTGATTEVS RTDVTSSSST SFPGPDQSTV SLDISTETNT RLSTSPIMTE SAEITITTQT
            4990 5000 5010 5020 5030 5040
GPHGATSQDT FTMDPSNTTP QAGIHSAMTH GFSQLDVTTL MSRIPQDVSW TSPPSVDKTS
            5050 5060 5070 5080 5090 5100
SPSSFLSSPA MTTPSLISST LPEDKLSSPM TSLLTSGLVK ITDILRTRLE PVTSSLPNFS
            5110 5120 5130 5140 5150 5160
STSDKILATS KDSKDTKEIF PSINTEETNV KANNSGHESH SPALADSETP KATTQMVITT
            5170 5180 5190 5200 5210 5220
TVGDPAPSTS MPVHGSSETT NIKREPTYFL TPRLRETSTS QESSFPTDTS FLLSKVPTGT
            5230 5240 5250 5260 5270 5280
ITEVSSTGVN SSSKISTPDH DKSTVPPDTF TGEIPRVFTS SIKTKSAEMT ITTQASPPES
            5290 5300 5310 5320 5330 5340
ASHSTLPLDT STTLSQGGTH STVTQGFPYS EVTTLMGMGP GNVSWMTTPP VEETSSVSSL
            5350 5360 5370 5380 5390 5400
MSSPAMTSPS PVSSTSPQSI PSSPLPVTAL PTSVLVTTTD VLGTTSPESV TSSPPNLSSI
            5410 5420 5430 5440 5450 5460
THERPATYKD TAHTEAAMHH STNTAVTNVG TSGSGHKSQS SVLADSETSK ATPLMSTTST
            5470 5480 5490 5500 5510 5520
LGDTSVSTST PNISQTNQIQ TEPTASLSPR LRESSTSEKT SSTTETNTAF SYVPTGAITQ
            5530 5540 5550 5560 5570 5580
ASRTEISSSR TSISDLDRPT IAPDISTGMI TRLFTSPIMT KSAEMTVTTQ TTTPGATSQG
            5590 5600 5610 5620 5630 5640
ILPWDTSTTL FQGGTHSTVS QGFPHSEITT LRSRTPGDVS WMTTPPVEET SSGFSLMSPS
            5650 5660 5670 5680 5690 5700
MTSPSPVSST SPESIPSSPL PVTALLTSVL VTTTNVLGTT SPETVTSSPP NLSSPTQERL
            5710 5720 5730 5740 5750 5760
TTYKDTAHTE AMHASMHTNT AVANVGTSIS GHESQSSVPA DSHTSKATSP MGITFAMGDT
            5770 5780 5790 5800 5810 5820
SVSTSTPAFF ETRIQTESTS SLIPGLRDTR TSEEINTVTE TSTVLSEVPT TTTTEVSRTE
            5830 5840 5850 5860 5870 5880
VITSSRTTIS GPDHSKMSPY ISTETITRLS TFPFVTGSTE MAITNQTGPI GTISQATLTL
            5890 5900 5910 5920 5930 5940
DTSSTASWEG THSPVTQRFP HSEETTTMSR STKGVSWQSP PSVEETSSPS SPVPLPAITS
            5950 5960 5970 5980 5990 6000
HSSLYSAVSG SSPTSALPVT SLLTSGRRKT IDMLDTHSEL VTSSLPSASS FSGEILTSEA
            6010 6020 6030 6040 6050 6060
STNTETIHFS ENTAETNMGT TNSMHKLHSS VSIHSQPSGH TPPKVTGSMM EDAIVSTSTP
            6070 6080 6090 6100 6110 6120
GSPETKNVDR DSTSPLTPEL KEDSTALVMN STTESNTVFS SVSLDAATEV SRAEVTYYDP
            6130 6140 6150 6160 6170 6180
TFMPASAQST KSPDISPEAS SSHSNSPPLT ISTHKTIATQ TGPSGVTSLG QLTLDTSTIA
            6190 6200 6210 6220 6230 6240
TSAGTPSART QDFVDSETTS VMNNDLNDVL KTSPFSAEEA NSLSSQAPLL VTTSPSPVTS
            6250 6260 6270 6280 6290 6300
TLQEHSTSSL VSVTSVPTPT LAKITDMDTN LEPVTRSPQN LRNTLATSEA TTDTHTMHPS
            6310 6320 6330 6340 6350 6360
INTAMANVGT TSSPNEFYFT VSPDSDPYKA TSAVVITSTS GDSIVSTSMP RSSAMKKIES
            6370 6380 6390 6400 6410 6420
ETTFSLIFRL RETSTSQKIG SSSDTSTVFD KAFTAATTEV SRTELTSSSR TSIQGTEKPT
            6430 6440 6450 6460 6470 6480
MSPDTSTRSV TMLSTFAGLT KSEERTIATQ TGPHRATSQG TLTWDTSITT SQAGTHSAMT
            6490 6500 6510 6520 6530 6540
HGFSQLDLST LTSRVPEYIS GTSPPSVEKT SSSSSLLSLP AITSPSPVPT TLPESRPSSP
            6550 6560 6570 6580 6590 6600
VHLTSLPTSG LVKTTDMLAS VASLPPNLGS TSHKIPTTSE DIKDTEKMYP STNIAVTNVG
            6610 6620 6630 6640 6650 6660
TTTSEKESYS SVPAYSEPPK VTSPMVTSFN IRDTIVSTSM PGSSEITRIE MESTFSVAHG
            6670 6680 6690 6700 6710 6720
LKGTSTSQDP IVSTEKSAVL HKLTTGATET SRTEVASSRR TSIPGPDHST ESPDISTEVI
            6730 6740 6750 6760 6770 6780
PSLPISLGIT ESSNMTIITR TGPPLGSTSQ GTFTLDTPTT SSRAGTHSMA TQEFPHSEMT
            6790 6800 6810 6820 6830 6840
TVMNKDPEIL SWTIPPSIEK TSFSSSLMPS PAMTSPPVSS TLPKTIHTTP SPMTSLLTPS
            6850 6860 6870 6880 6890 6900
LVMTTDTLGT SPEPTTSSPP NLSSTSHVIL TTDEDTTAIE AMHPSTSTAA TNVETTCSGH
            6910 6920 6930 6940 6950 6960
GSQSSVLTDS EKTKATAPMD TTSTMGHTTV STSMSVSSET TKIKRESTYS LTPGLRETSI
            6970 6980 6990 7000 7010 7020
SQNASFSTDT SIVLSEVPTG TTAEVSRTEV TSSGRTSIPG PSQSTVLPEI STRTMTRLFA
            7030 7040 7050 7060 7070 7080
SPTMTESAEM TIPTQTGPSG STSQDTLTLD TSTTKSQAKT HSTLTQRFPH SEMTTLMSRG
            7090 7100 7110 7120 7130 7140
PGDMSWQSSP SLENPSSLPS LLSLPATTSP PPISSTLPVT ISSSPLPVTS LLTSSPVTTT
            7150 7160 7170 7180 7190 7200
DMLHTSPELV TSSPPKLSHT SDERLTTGKD TTNTEAVHPS TNTAASNVEI PSFGHESPSS
            7210 7220 7230 7240 7250 7260
ALADSETSKA TSPMFITSTQ EDTTVAISTP HFLETSRIQK ESISSLSPKL RETGSSVETS
            7270 7280 7290 7300 7310 7320
SAIETSAVLS EVSIGATTEI SRTEVTSSSR TSISGSAEST MLPEISTTRK IIKFPTSPIL
            7330 7340 7350 7360 7370 7380
AESSEMTIKT QTSPPGSTSE STFTLDTSTT PSLVITHSTM TQRLPHSEIT TLVSRGAGDV
            7390 7400 7410 7420 7430 7440
PRPSSLPVEE TSPPSSQLSL SAMISPSPVS STLPASSHSS SASVTSPLTP GQVKTTEVLD
            7450 7460 7470 7480 7490 7500
ASAEPETSSP PSLSSTSVEI LATSEVTTDT EKIHPFPNTA VTKVGTSSSG HESPSSVLPD
            7510 7520 7530 7540 7550 7560
SETTKATSAM GTISIMGDTS VSTLTPALSN TRKIQSEPAS SLTTRLRETS TSEETSLATE
            7570 7580 7590 7600 7610 7620
ANTVLSKVST GATTEVSRTE AISFSRTSMS GPEQSTMSQD ISIGTIPRIS ASSVLTESAK
            7630 7640 7650 7660 7670 7680
MTITTQTGPS ESTLESTLNL NTATTPSWVE THSIVIQGFP HPEMTTSMGR GPGGVSWPSP
            7690 7700 7710 7720 7730 7740
PFVKETSPPS SPLSLPAVTS PHPVSTTFLA HIPPSPLPVT SLLTSGPATT TDILGTSTEP
            7750 7760 7770 7780 7790 7800
GTSSSSSLST TSHERLTTYK DTAHTEAVHP STNTGGTNVA TTSSGYKSQS SVLADSSPMC
            7810 7820 7830 7840 7850 7860
TTSTMGDTSV LTSTPAFLET RRIQTELASS LTPGLRESSG SEGTSSGTKM STVLSKVPTG
            7870 7880 7890 7900 7910 7920
ATTEISKEDV TSIPGPAQST ISPDISTRTV SWFSTSPVMT ESAEITMNTH TSPLGATTQG
            7930 7940 7950 7960 7970 7980
TSTLATSSTT SLTMTHSTIS QGFSHSQMST LMRRGPEDVS WMSPPLLEKT RPSFSLMSSP
            7990 8000 8010 8020 8030 8040
ATTSPSPVSS TLPESISSSP LPVTSLLTSG LAKTTDMLHK SSEPVTNSPA NLSSTSVEIL
            8050 8060 8070 8080 8090 8100
ATSEVTTDTE KTHPSSNRTV TDVGTSSSGH ESTSFVLADS QTSKVTSPMV ITSTMEDTSV
            8110 8120 8130 8140 8150 8160
STSTPGFFET SRIQTEPTSS LTLGLRKTSS SEGTSLATEM STVLSGVPTG ATAEVSRTEV
            8170 8180 8190 8200 8210 8220
TSSSRTSISG FAQLTVSPET STETITRLPT SSIMTESAEM MIKTQTDPPG STPESTHTVD
            8230 8240 8250 8260 8270 8280
ISTTPNWVET HSTVTQRFSH SEMTTLVSRS PGDMLWPSQS SVEETSSASS LLSLPATTSP
            8290 8300 8310 8320 8330 8340
SPVSSTLVED FPSASLPVTS LLTPGLVITT DRMGISREPG TSSTSNLSST SHERLTTLED
            8350 8360 8370 8380 8390 8400
TVDTEDMQPS THTAVTNVRT SISGHESQSS VLSDSETPKA TSPMGTTYTM GETSVSISTS
            8410 8420 8430 8440 8450 8460
DFFETSRIQI EPTSSLTSGL RETSSSERIS SATEGSTVLS EVPSGATTEV SRTEVISSRG
            8470 8480 8490 8500 8510 8520
TSMSGPDQFT ISPDISTEAI TRLSTSPIMT ESAESAITIE TGSPGATSEG TLTLDTSTTT
            8530 8540 8550 8560 8570 8580
FWSGTHSTAS PGFSHSEMTT LMSRTPGDVP WPSLPSVEEA SSVSSSLSSP AMTSTSFFSA
            8590 8600 8610 8620 8630 8640
LPESISSSPH PVTALLTLGP VKTTDMLRTS SEPETSSPPN LSSTSAEILA TSEVTKDREK
            8650 8660 8670 8680 8690 8700
IHPSSNTPVV NVGTVIYKHL SPSSVLADLV TTKPTSPMAT TSTLGNTSVS TSTPAFPETM
            8710 8720 8730 8740 8750 8760
MTQPTSSLTS GLREISTSQE TSSATERSAS LSGMPTGATT KVSRTEALSL GRTSTPGPAQ
            8770 8780 8790 8800 8810 8820
STISPEISTE TITRISTPLT TTGSAEMTIT PKTGHSGASS QGTFTLDTSS RASWPGTHSA
            8830 8840 8850 8860 8870 8880
ATHRSPHSGM TTPMSRGPED VSWPSRPSVE KTSPPSSLVS LSAVTSPSPL YSTPSESSHS
            8890 8900 8910 8920 8930 8940
SPLRVTSLFT PVMMKTTDML DTSLEPVTTS PPSMNITSDE SLATSKATME TEAIQLSENT
            8950 8960 8970 8980 8990 9000
AVTQMGTISA RQEFYSSYPG LPEPSKVTSP VVTSSTIKDI VSTTIPASSE ITRIEMESTS
            9010 9020 9030 9040 9050 9060
TLTPTPRETS TSQEIHSATK PSTVPYKALT SATIEDSMTQ VMSSSRGPSP DQSTMSQDIS
            9070 9080 9090 9100 9110 9120
SEVITRLSTS PIKAESTEMT ITTQTGSPGA TSRGTLTLDT STTFMSGTHS TASQGFSHSQ
            9130 9140 9150 9160 9170 9180
MTALMSRTPG DVPWLSHPSV EEASSASFSL SSPVMTSSSP VSSTLPDSIH SSSLPVTSLL
            9190 9200 9210 9220 9230 9240
TSGLVKTTEL LGTSSEPETS SPPNLSSTSA EILATTEVTT DTEKLEMTNV VTSGYTHESP
            9250 9260 9270 9280 9290 9300
SSVLADSVTT KATSSMGITY PTGDTNVLTS TPAFSDTSRI QTKSKLSLTP GLMETSISEE
            9310 9320 9330 9340 9350 9360
TSSATEKSTV LSSVPTGATT EVSRTEAISS SRTSIPGPAQ STMSSDTSME TITRISTPLT
            9370 9380 9390 9400 9410 9420
RKESTDMAIT PKTGPSGATS QGTFTLDSSS TASWPGTHSA TTQRFPQSVV TTPMSRGPED
            9430 9440 9450 9460 9470 9480
VSWPSPLSVE KNSPPSSLVS SSSVTSPSPL YSTPSGSSHS SPVPVTSLFT SIMMKATDML
            9490 9500 9510 9520 9530 9540
DASLEPETTS APNMNITSDE SLATSKATTE TEAIHVFENT AASHVETTSA TEELYSSSPG
            9550 9560 9570 9580 9590 9600
FSEPTKVISP VVTSSSIRDN MVSTTMPGSS GITRIEIESM SSLTPGLRET RTSQDITSST
            9610 9620 9630 9640 9650 9660
ETSTVLYKMS SGATPEVSRT EVMPSSRTSI PGPAQSTMSL DISDEVVTRL STSPIMTESA
            9670 9680 9690 9700 9710 9720
EITITTQTGY SLATSQVTLP LGTSMTFLSG THSTMSQGLS HSEMTNLMSR GPESLSWTSP'
            9730 9740 9750 9760 9770 9780
RFVETTRSSS SLTSLPLTTS LSPVSSTLLD SSPSSPLPVT SLILPGLVKT TEVLDTSSEP
            9790 9800 9810 9820 9830 9840
KTSSSPNLSS TSVEIPATSE IMTDTEKIHP SSNTAVAKVR TSSSVHESHS SVLADSETTI
            9850 9860 9870 9880 9890 9900
TIPSMGITSA VDDTTVFTSN PAFSETRRIP TEPTFSLTPG FRETSTSEET TSITETSAVL
            9910 9920 9930 9940 9950 9960
YGVPTSATTE VSMTEIMSSN RTHIPDSDQS TMSPDIITEV ITRLSSSSMM SESTQMTITT
            9970 9980 9990 10000 10010 10020
QKSSPGATAQ STLTLATTTA PLARTHSTVP PRFLHSEMTT LMSRSPENPS WKSSPFVEKT
          10030 10040 10050 10060 10070 10080
SSSSSLLSLP VTTSPSVSST LPQSIPSSSF SVTSLLTPGM VKTTDTSTEP GTSLSPNLSG
          10090 10100 10110 10120 10130 10140
TSVEILAASE VTTDTEKIHP SSSMAVTNVG TTSSGHELYS SVSIHSEPSK ATYPVGTPSS
          10150 10160 10170 10180 10190 10200
MAETSISTSM PANFETTGFE AEPFSHLTSG FRKTNMSLDT SSVTPTNTPS SPGSTHLLQS
          10210 10220 10230 10240 10250 10260
SKTDFTSSAK TSSPDWPPAS QYTEIPVDII TPFNASPSIT ESTGITSFPE SRFTMSVTES
          10270 10280 10290 10300 10310 10320
THHLSTDLLP SAETISTGTV MPSLSEAMTS FATTGVPRAI SGSGSPFSRT ESGPGDATLS
          10330 10340 10350 10360 10370 10380
TIAESLPSST PVPFSSSTFT TTDSSTIPAL HEITSSSATP YRVDTSLGTE SSTTEGRLVM
          10390 10400 10410 10420 10430 10440
VSTLDTSSQP GRTSSTPILD TRMTESVELG TVTSAYQVPS LSTRLTRTDG IMEHITKIPN
          10450 10460 10470 10480 10490 10500
EAAHRGTIRP VKGPQTSTSP ASPKGLHTGG TKRMETTTTA LKTTTTALKT TSRATLTTSV
          10510 10520 10530 10540 10550 10560
YTPTLGTLTP LNASRQMAST ILTEMMITTP YVFPDVPETT SSLATSLGAE TSTALPRTTP
          10570 10580 10590 10600 10610 10620
SVLNRESETT ASLVSRSGAE RSPVIQTLDV SSSEPDTTAS WVIHPAETIP TVSKTTPNFF
          10630 10640 10650 10660 10670 10680
HSELDTVSST ATSHGADVSS AIPTNISPSE LDALTPLVTI SGTDTSTTFP TLTKSPHETE
          10690 10700 10710 10720 10730 10740
TRTTWLTHPA ETSSTIPRTI PNFSHHESDA TPSIATSPGA ETSSAIPIMT VSPGAEDLVT
          10750 10760 10770 10780 10790 10800
SQVTSSGTDR NMTIPTLTLS PGEPKTIASL VTHPEAQTSS AIPTSTISPA VSRLVTSMVT
          10810 10820 10830 10840 10850 10860
SLAAKTSTTN RALTNSPGEP ATTVSLVTHP AQTSPTVPWT TSIFFHSKSD TTPSMTTSHG
          10870 10880 10890 10900 10910 10920
AESSSAVPTP TVSTEVPGVV TPLVTSSRAV ISTTIPILTL SPGEPETTPS MATSHGEEAS
          10930 10940 10950 10960 10970 10980
SAIPTPTVSP GVPGVVTSLV TSSRAVTSTT IPILTFSLGE PETTPSMATS HGTEAGSAVP
          10990 11000 11010 11020 11030 11040
TVLPEVPGMV TSLVASSRAV TSTTLPTLTL SPGEPETTPS MATSHGAEAS STVPTVSPEV
          11050 11060 11070 11080 11090 11100
PGVVTSLVTS SSGVNSTSIP TLILSPGELE TTPSMATSHG AEASSAVPTP TVSPGVSGVV
          11110 11120 11130 11140 11150 11160
TPLVTSSRAV TSTTIPILTL SSSEPETTPS MATSHGVEAS SAVLTVSPEV PGMVTSLVTS
          11170 11180 11190 11200 11210 11220
SRAVTSTTIP TLTISSDEPE TTTSLVTHSE AKMISAIPTL AVSPTVQGLV TSLVTSSGSE
          11230 11240 11250 11260 11270 11280
TSAFSNLTVA SSQPETIDSW VAHPGTEASS VVPTLTVSTG EPFTNISLVT HPAESSSTLP
          11290 11300 11310 11320 11330 11340
RTTSRFSHSE LDTMPSTVTS PEAESSSAIS TTISPGIPGV LTSLVTSSGR DISATFPTVP
          11350 11360 11370 11380 11390 11400
ESPHESEATA SWVTHPAVTS TTVPRTTPNY SHSEPDTTPS IATSPGAEAT SDFPTITVSP
          11410 11420 11430 11440 11450 11460
DVPDMVTSQV TSSGTDTSIT IPTLTLSSGE PETTTSFITY SETHTSSAIP TLPVSPGASK
          11470 11480 11490 11500 11510 11520
MLTSLVISSG TDSTTTFPTL TETPYEPETT AIQLIHPAET NTMVPKTTPK FSHSKSDTTL
          11530 11540 11550 11560 11570 11580
PVAITSPGPE ASSAVSTTTI SPDMSDLVTS LVPSSGTDTS TTFPTLSETP YEPETTVTWL
          11590 11600 11610 11620 11630 11640
THPAETSTTV SGTIPNFSHR GSDTAPSMVT SPGVDTRSGV PTTTIPPSIP GVVTSQVTSS
          11650 11660 11670 11680 11690 11700
ATDTSTAIPT LTPSPGEPET TASSATHPGT QTGFTVPIRT VPSSEPDTMA SWVTHPPQTS
          11710 11720 11730 11740 11750 11760
TPVSRTTSSF SHSSPDATPV MATSPRTEAS SAVLTTISPG APEMVTSQIT SSGAATSTTV
          11770 11780 11790 11800 11810 11820
PTLTHSPGMP ETTALLSTHP RTGTSKTFPA STVFPQVSET TASLTIRPGA ETSTALPTQT
          11830 11840 11850 11860 11870 11880
TSSLFTLLVT GTSRVDLSPT ASPGVSAKTA PLSTHPGTET STMIPTSTLS LGLLETTGLL
          11890 11900 11910 11920 11930 11940
ATSSSAETST STLTLTVSPA VSGLSSASIT TDKPQTVTSW NTETSPSVTS VGPPEFSRTV
          11950 11960 11970 11980 11990 12000
TGTTMTLIPS EMPTPPKTSH GEGVSPTTIL RTTMVEATNL ATTGSSPTVA KTTTTFNTLA
          12010 12020 12030 12040 12050 12060
GSLFTPLTTP GMSTLASESV TSRTSYNHRS WISTTSSYNR RYWTPATSTP VTSTFSPGIS
          12070 12080 12090 12100 12110 12120
TSSIPSSTAA TVPFMVPFTL NFTITNLQYE EDMRHPGSRK FNATERELQG LLKPLFRNSS
          12130 12140 12150 12160 12170 12180
LEYLYSGCRL ASLRPEKDSS AMAVDAICTH RPDPEDLGLD RERLYWELSN LTNGIQELGP
          12190 12200 12210 12220 12230 12240
YTLDRNSLYV NGFTHRSSMP TTSTPGTSTV DVGTSGTPSS SPSPTAAGPL LMPFTLNFTI
          12250 12260 12270 12280 12290 12300
TNLQYEEDMR RTGSRKFNTM ESVLQGLLKP LFKNTSVGPL YSGCRLTLLR PEKDGAATGV
          12310 12320 12330 12340 12350 12360
DAICTHRLDP KSPGLNREQL YWELSKLTND IEELGPYTLD RNSLYVNGFT HQSSVSTTST
          12370 12380 12390 12400 12410 12420
PGTSTVDLRT SGTPSSLSSP TIMAAGPLLV PFTLNFTITN LQYGEDMGHP GSRKFNTTER
          12430 12440 12450 12460 12470 12480
VLQGLLGPIF KNTSVGPLYS GCRLTSLRSE KDGAATGVDA ICIHHLDPKS PGLNRERLYW
          12490 12500 12510 12520 12530 12540
ELSQLTNGIK ELGPYTLDRN SLYVNGFTHR TSVPTTSTPG TSTVDLGTSG TPFSLPSPAT
          12550 12560 12570 12580 12590 12600
AGPLLVLFTL NFTITNLKYE EDMHRPGSRK FNTTERVLQT LLGPMFKNTS VGLLYSGCRL
          12610 12620 12630 12640 12650 12660
TLLRSEKDGA ATGVDAICTH RLDPKSPGLD REQLYWELSQ LTNGIKELGP YTLDRNSLYV
          12670 12680 12690 12700 12710 12720
NGFTHWIPVP TSSTPGTSTV DLGSGTPSSL PSPTAAGPLL VPFTLNFTIT NLQYEEDMHH
          12730 12740 12750 12760 12770 12780
PGSRKFNTTE RVLQGLLGPM FKNTSVGLLY SGCRLTLLRS EKDGAATGVD AICTHRLDPK
          12790 12800 12810 12820 12830 12840
SPGVDREQLY WELSQLTNGI KELGPYTLDR NSLYVNGFTH QTSAPNTSTP GTSTVDLGTS
          12850 12860 12870 12880 12890 12900
GTPSSLPSPT SAGPLLVPFT LNFTITNLQY EEDMRHPGSR KFNTTERVLQ GLLKPLFKST
          12910 12920 12930 12940 12950 12960
SVGPLYSGCR LTLLRSEKDG AATGVDAICT HRLDPKSPGV DREQLYWELS QLTNGIKELG
          12970 12980 12990 13000 13010 13020
PYTLDRNSLY VNGFTHQTSA PNTSTPGTST VDLGTSGTPS SLPSPTSAGP LLVPFTLNFT
          13030 13040 13050 13060 13070 13080
ITNLQYEEDM HHPGSRKFNT TERVLQGLLG PMFKNTSVGL LYSGCRLTLL RPEKNGAATG
          13090 13100 13110 13120 13130 13140
MDAICSHRLD PKSPGLNREQ LYWELSQLTH GIKELGPYTL DRNSLYVNGF THRSSVAPTS
          13150 13160 13170 13180 13190 13200
TPGTSTVDLG TSGTPSSLPS PTTAVPLLVP FTLNFTITNL QYGEDMRHPG SRKFNTTERV
          13210 13220 13230 13240 13250 13260
LQGLLGPLFK NSSVGPLYSG CRLISLRSEK DGAATGVDAI CTHHLNPQSP GLDREQLYWQ
          13270 13280 13290 13300 13310 13320
LSQMTNGIKE LGPYTLDRNS LYVNGFTHRS SGLTTSTPWT STVDLGTSGT PSPVPSPTTA
          13330 13340 13350 13360 13370 13380
GPLLVPFTLN FTITNLQYEE DMHRPGSRKF NTTERVLQGL LSPIFKNSSV GPLYSGCRLT
          13390 13400 13410 13420 13430 13440
SLRPEKDGAA TGMDAVCLYH PNPKRPGLDR EQLYWELSQL THNITELGPY SLDRDSLYVN
          13450 13460 13470 13480 13490 13500
GFTHQNSVPT TSTPGTSTVY WATTGTPSSF PGHTEPGPLL IPFTFNFTIT NLHYEENMQH
          13510 13520 13530 13540 13550 13560
PGSRKFNTTE RVLQGLLKPL FKNTSVGPLY SGCRLTSLRP EKDGAATGMD AVCLYHPNPK
          13570 13580 13590 13600 13610 13620
RPGLDREQLY WELSQLTHNI TELGPYSLDR DSLYVNGFTH QNSVPTTSTP GTSTVYWATT
          13630 13640 13650 13660 13670 13680
GTPSSFPGHT EPGPLLIPFT FNFTITNLHY EENMQHPGSR KFNTTERVLQ GLLKPLFKNT
          13690 13700 13710 13720 13730 13740
SVGPLYSGCR LTLLRPEKHE AATGVDTICT HRVDPIGPGL DRERLYWELS QLTNSITELG
          13750 13760 13770 13780 13790 13800
PYTLDRDSLY VNGFNPRSSV PTTSTPGTST VHLATSGTPS SLPGHTAPVP LLIPFTLNFT
          13810 13820 13830 13840 13850 13860
ITNLHYEENM QHPGSRKFNT TERVLQGLLK PLFKNTSVGP LYSGCRLTLL RPEKHEAATG
          13870 13880 13890 13900 13910 13920
VDTICTHRVD PIGPGLXXEX LYWELSXLTX XIXELGPYTL DRXSLYVNGF THXXSXPTTS
          13930 13940 13950 13960 13970 13980
TPGTSTVXXG TSGTPSSXPX XTSAGPLLVP FTLNFTITNL QYEEDMHHPG SRKFNTTERV
          13990 14000 14010 14020 14030 14040
LQGLLGPMFK NTSVGLLYSG CRLTLLRPEK NGAATGMDAI CSHRLDPKSP GLDREQLYWE
          14050 14060 14070 14080 14090 14100
LSQLTHGIKE LGPYTLDRNS LYVNGFTHRS SVAPTSTPGT STVDLGTSGT PSSLPSPTTA
          14110 14120 14130 14140 14150 14160
VPLLVPFTLN FTITNLQYGE DMRHPGSRKF NTTERVLQGL LGPLFKNSSV GPLYSGCRLI
          14170 14180 14190 14200 14210 14220
SLRSEKDGAA TGVDAICTHH LNPQSPGLDR EQLYWQLSQM TNGIKELGPY TLDRNSLYVN
          14230 14240 14250 14260 14270 14280
GFTHRSSGLT TSTPWTSTVD LGTSGTPSPV PSPTTAGPLL VPFTLNFTIT NLQYEEDMHR
          14290 14300 14310 14320 14330 14340
PGSRKFNATE RVLQGLLSPI FKNSSVGPLY SGCRLTSLRP EKDGAATGMD AVCLYHPNPK
          14350 14360 14370 14380 14390 14400
RPGLDREQLY WELSQLTHNI TELGPYSLDR DSLYVNGFTH QSSMTTTRTP DTSTMHLATS
          14410 14420 14430 14440 14450 14460
RTPASLSGPT TASPLLVLFT INCTITNLQY EEDMRRTGSR KFNTMESVLQ GLLKPLFKNT
          14470 14480 14490 14500 14510 14520
SVGPLYSGCR LTLLRPKKDG AATGVDAICT HRLDPKSPGL NREQLYWELS KLTNDIEELG
          14530 14540 14550 14560 14570 14580
PYTLDRNSLY VNGFTHQSSV STTSTPGTST VDLRTSGTPS SLSSPTIMXX XPLLXPFTXN
          14590 14600 14610 14620 14630 14640
XTITNLXXXX XMXXPGSRKF NTTERVLQGL LRPLFKNTSV SSLYSGCRLT LLRPEKDGAA
          14650 14660 14670 14680 14690 14700
TRVDAACTYR PDPKSPGLDR EQLYWELSQL THSITELGPY TLDRVSLYVN GFNPRSSVPT
          14710 14720 14730 14740 14750 14760
TSTPGTSTVH LATSGTPSSL PGHTXXXPLL XPFTXNXTIT NLXXXXXMXX PGSRKFNTTE
          14770 14780 14790 14800 14810 14820
RVLQGLLKPL FRNSSLEYLY SGCRLASLRP EKDSSAMAVD AICTHRPDPE DLGLDRERLY
          14830 14840 14850 14860 14870 14880
WELSNLTNGI QELGPYTLDR NSLYVNGFTH RSSGLTTSTP WTSTVDLGTS GTPSPVPSPT
          14890 14900 14910 14920 14930 14940
TAGPLLVPFT LNFTITNLQY EEDMHRPGSR RFNTTERVLQ GLLTPLFKNT SVGPLYSGCR
          14950 14960 14970 14980 14990 15000
LTLLRPEKQE AATGVDTICT HRVDPIGPGL DRERLYWELS QLTNSITELG PYTLDRDSLY
          15010 15020 15030 15040 15050 15060
VNGFNPWSSV PTTSTPGTST VHLATSGTPS SLPGHTAPVP LLIPFTLNFT ITDLHYEENM
          15070 15080 15090 15100 15110 15120
QHPGSRKFNT TERVLQGLLK PLFKSTSVGP LYSGCRLTLL RPEKHGAATG VDAICTLRLD
          15130 15140 15150 15160 15170 15180
PTGPGLDRER LYWELSQLTN SVTELGPYTL DRDSLYVNGF THRSSVPTTS IPGTSAVHLE
          15190 15200 15210 15220 15230 15240
TSGTPASLPG HTAPGPLLVP FTLNFTITNL QYEEDMRHPG SRKFSTTERV LQGLLKPLFK
          15250 15260 15270 15280 15290 15300
NTSVSSLYSG CRLTLLRPEK DGAATRVDAV CTHRPDPKSP GLDRERLYWK LSQLTHGITE
          15310 15320 15330 15340 15350 15360
LGPYTLDRHS LYVNGFTHQS SMTTTRTPDT STMHLATSRT PASLSGPTTA SPLLVLFTIN
          15370 15380 15390 15400 15410 15420
FTITNLRYEE NMHHPGSRKF NTTERVLQGL LRPVFKNTSV GPLYSGCRLT TLRPKKDGAA
          15430 15440 15450 15460 15470 15480
TKVDAICTYR PDPKSPGLDR EQLYWELSQL THSITELGPY TQDRDSLYVN GFTHRSSVPT
          15490 15500 15510 15520 15530 15540
TSIPGTSAVH LETSGTPASL PGHTAPGPLL VPFTLNFTIT NLQYEEDMRH PGSRKFNTTE
          15550 15560 15570 15580 15590 15600
RVLQGLLKPL FKSTSVGPLY SGCRLTLLRP EKRGAATGVD TICTHRLDPL NPGLDREQLY
          15610 15620 15630 15640 15650 15660
WELSKLTRGI IELGPYLLDR GSLYVNGFTH RTSVPTTSTP GTSTVDLGTS GTPFSLPSPA
          15670 15680 15690 15700 15710 15720
XXXPLLXPFT XNXTITNLXX XXXMXXPGSR KFNTTERVLQ TLLGPMFKNT SVGLLYSGCR
          15730 15740 15750 15760 15770 15780
LTLLRSEKDG AATGVDAICT HRLDPKSPGV DREQLYWELS QLTNGIKELG PYTLDRNSLY
          15790 15800 15810 15820 15830 15840
VNGFTHWIPV PtSSTPGTST VDLGSGTPSS LPSPTTAGPL LVPFTLNFTI TNLKYEEDMH
          15850 15860 15870 15880 15890 15900
CPGSRKFNTT ERVLQSLLGP MFKNTSVGPL YSGCRLTLLR SEKDGAATGV DAICTHRLDP
          15910 15920 15930 15940 15950 15960
KSPGVDREQL YWELSQLTNG IKELGPYTLD RNSLYVNGFT HQTSAPNTST PGTSTVDLGT
          15970 15980 15990 16000 16010 16020
SGTPSSLPSP TXXXPLLXPF TXNXTITNLX XXXXMXXPGS RKFNTTEXVL QGLLXPXFKN
          16030 16040 16050 16060 16070 16080
XSVGXLYSGC RLTXLRXEKX GAATGXDAIC XHXXXPKXPG LXXEXLYWEL SXLTXXIXEL
          16090 16100 16110 16120 16130 16140
GPYTLDRXSL YVNGFTHWIP VPTSSTPGTS TVDLGSGTPS SLPSPTTAGP LLVPFTLNFT
          16150 16160 16170 16180 16190 16200
ITNLKYEEDM HCPGSRKFNT TERVLQSLLG PMFKNTSVGP LYSGCRLTSL RSEKDGAATG
          16210 16220 16230 16240 16250 16260
VDAICTHRVD PKSPGVDREQ LYWELSQLTN GIKELGPYTL DRNSLYVNGF THQTSAPNTS
          16270 16280 16290 16300 16310 16320
TPGTSTVXXG TSGTPSSXPX XTSAGPLLVP FTLNFTITNL QYEEDMHHPG SRKFNTTERV
          16330 16340 16350 16360 16370 16380
LQGLLGPMFK NTSVGLLYSG CRLTLLRPEK NGATTGMDAI CTHRLDPKSP GLXXEXLYWE
          16390 16400 16410 16420 16430 16440
LSXLTXXIXE LGPYTLDRXS LYVNGFTHXX SXPTTSTPGT STVXXGTSGT PSSXPXXTXX
          16450 16460 16470 16480 16490 16500
XPLLXPFTXN XTITNLXXXX XMXXPGSRKF NTTERVLQGL LKPLFRNSSL EYLYSGCRLA
          16510 16520 16530 16540 16550 16560
SLRPEKDSSA MAVDAICTHR PDPEDLGLDR ERLYWELSNL TNGIQELGPY TLDRNSLYVN
          16570 16580 16590 16600 16610 16620
GFTHRSSMPT TSTPGTSTVD VGTSGTPSSS PSPTTAGPLL IPFTLNFTIT NLQYGEDMGH
          16630 16640 16650 16660 16670 16680
PGSRKFNTTE RVLQGLLGPI FKNTSVGPLY SGCRLTSLRS EKDGAATGVD AICIHHLDPK
          16690 16700 16710 16720 16730 16740
SPGLNRERLY WELSQLTNGI KELGPYTLDR NSLYVNGFTH RTSVPTTSTP GTSTVDLGTS
          16750 16760 16770 16780 16790 16800
GTPFSLPSPA TAGPLLVLFT LNFTITNLKY EEDMHRPGSR KFNTTERVLQ TLLGPMFKNT
          16810 16820 16830 16840 16850 16860
SVGLLYSGCR LTLLRSEKDG AATGVDAICT HRLDPKSPGL XXEXLYWELS XLTXXIXELG
          16870 16880 16890 16900 16910 16920
PYTLDRXSLY VNGFTHXXSX PTTSTPGTST VXXGTSGTPS SXPXXTXXXP LLXPFTXNXT
          16930 16940 16950 16960 16970 16980
ITNLXXXXXM XXPGSRKFNT TERVLQGLLR PVFKNTSVGP LYSGCRLTLL RPKKDGAATK
          16990 17000 17010 17020 17030 17040
VDAICTYRPD PKSPGLDREQ LYWELSQLTH SITELGPYTQ DRDSLYVNGF THRSSVPTTS
          17050 17060 17070 17080 17090 17100
IPGTSAVHLE TTGTPSSFPG HTEPGPLLIP FTFNFTITNL RYEENMQHPG SRKFNTTERV
          17110 17120 17130 17140 17150 17160
LQGLLTPLFK NTSVGPLYSG CRLTLLRPEK QEAATGVDTI CTHRVDPIGP GLDRERLYWE
          17170 17180 17190 17200 17210 17220
LSQLTNSITE LGPYTLDRDS LYVDGFNPWS SVPTTSTPGT STVHLATSGT PSPLPGHTAP
          17230 17240 17250 17260 17270 17280
VPLLIPFTLN FTITDLHYEE NMQHPGSRKF NTTERVLQGL LKPLFKSTSV GPLYSGCRLT
          17290 17300 17310 17320 17330 17340
LLRPEKHGAA TGVDAICTLR LDPTGPGLDR ERLYWELSQL TNSITELGPY TLDRDSLYVN
          17350 17360 17370 17380 17390 17400
GFNPWSSVPT TSTPGTSTVH LATSGTPSSL PGHTTAGPLL VPFTLNFTIT NLKYEEDMHC
          17410 17420 17430 17440 17450 17460
PGSRKFNTTE RVLQSLHGPM FKNTSVGPLY SGCRLTLLRS EKDGAATGVD AICTHRLDPK
          17470 17480 17490 17500 17510 17520
SPGLXXEXLY WELSXLTXXI XELGPYTLDR XSLYVNGFTH XXSXPTTSTP GTSTVXXGTS
          17530 17540 17550 17560 17570 17580
GTPSSXPXXT XXXPLLXPFT XNXTITNLXX XXXMXXPGSR KFNTTEXVLQ GLLXPXFKNX
          17590 17600 17610 17620 17630 17640
SVGXLYSGCR LTXLRXEKXG AATGXDAICX HXXXPKXPGL XXEXLYWELS XLTNSITELG
          17650 17660 17670 17680 17690 17700
PYTLDRDSLY VNGFTHRSSM PTTSIPGTSA VHLETSGTPA SLPGHTAPGP LLVPFTLNFT
          17710 17720 17730 17740 17750 17760
ITNLQYEEDM RHPGSRKFNT TERVLQGLLK PLFKSTSVGP LYSGCRLTLL RPEKRGAATG
          17770 17780 17790 17800 17810 17820
VDTICTHRLD PLNPGLXXEX LYWELSXLTX XIXELGPYTL DRXSLYVNGF THXXSXPTTS
          17830 17840 17850 17860 17870 17880
TPGTSTVXXG TSGTPSSXPX XTXXXPLLXP FTXNXTITNL XXXXXMXXPG SRKFNTTEXV
          17890 17900 17910 17920 17930 17940
LQGLLXPXFK NXSVGXLYSG CRLTXLRXEK XGAATGXDAI CXHXXXPKXP GLXXEXLYWE
          17950 17960 17970 17980 17990 18000
LSXLTXXIXE LGPYTLDRXS LYVNGFHPRS SVPTTSTPGT STVHLATSGT PSSLPGHTAP
          18010 18020 18030 18040 18050 18060
VPLLIPFTLN FTITNLHYEE NMQHPGSRKF NTTERVLQGL LGPMFKNTSV GLLYSGCRLT
          18070 18080 18090 18100 18110 18120
LLRPEKNGAA TGMDAICSHR LDPKSPGLXX EXLYWELSXL TXXIXELGPY TLDRXSLYVN
          18130 18140 18150 18160 18170 18180
GFTHXXSXPT TSTPGTSTVX XGTSGTPSSX PXXTXXXPLL XPFTXNXTIT NLXXXXXMXX
          18190 18200 18210 18220 18230 18240
PGSRKFNTTE XVLQGLLXPX FKNXSVGXLY SGCRLTXLRX EKXGAATGXD AICXHXXXPK
          18250 18260 18270 18280 18290 18300
XPGLXXEXLY WELSXLTXXI XELGPYTLDR XSLYVNGFTH QNSVPTTSTP GTSTVYWATT
          18310 18320 18330 18340 18350 18360
GTPSSFPGHT EPGPLLIPFT FNFTITNLHY EENMQHPGSR KFNTTERVLQ GLLTPLFKNT
          18370 18380 18390 18400 18410 18420
SVGPLYSGCR LTLLRPEKQE AATGVDTICT HRVDPIGPGL XXEXLYWELS XLTXXIXELG
          18430 18440 18450 18460 18470 18480
PYTLDRXSLY VNGFTHXXSX PTTSTPGTST VXXGTSGTPS SXPXXTXXXP LLXPFTXNXT
          18490 18500 18510 18520 18530 18540
ITNLXXXXXM XXPGSRKFNT TEXVLQGLLX PXFKNXSVGX LYSGCRLTXL RXEKXGAATG
          18550 18560 18570 18580 18590 18600
XDAICXHXXX PKXPGLXXEX LYWELSXLTX XIXELGPYTL DRXSLYVNGF THRSSVPTTS
          18610 18620 18630 18640 18650 18660
SPGTSTVHLA TSGTPSSLPG HTAPVPLLIP FTLNFTITNL HYEENMQHPG SRKFNTTERV
          18670 18680 18690 18700 18710 18720
LQGLLKPLFK STSVGPLYSG CRLTLLRPEK HGAATGVDAI CTLRLDPTGP GLXXEXLYWE
          18730 18740 18750 18760 18770 18780
LSXLTXXIXE LGPYTLDRXS LYVNGFTHXX SXPTTSTPGT STVXXGTSGT PSSXPXXTXX
          18790 18800 18810 18820 18830 18840
XPLLXPFTXN XTITNLXXXX XMXXPGSRKF NTTEXVLQGL LXPXFKNXSV GXLYSGCRLT
          18850 18860 18870 18880 18890 18900
XLRXEKXGAA TGXDAICXHX XXPKXPGLXX EXLYWELSXL TXXIXELGPY TLDRXSLYVN
          18910 18920 18930 18940 18950 18960
GFTHRTSVPT TSTPGTSTVH LATSGTPSSL PGHTAPVPLL IPFTLNFTIT NLQYEEDMHR
          18970 18980 18990 19000 19010 19020
PGSRKFNTTE RVLQGLLSPI FKNSSVGPLY SGCRLTSLRP EKDGAATGMD AVCLYHPNPK
          19030 19040 19050 19060 19070 19080
RPGLDREQLY CELSQLTHNI TELGPYSLDR DSLYVNGFTH QNSVPTTSTP GTSTVYWATT
          19090 19100 19110 19120 19130 19140
GTPSSFPGHT XXXPLLXPFT XNXTITNLXX XXXMXXPGSR KFNTTEXVLQ GLLXPXFKNX
          19150 19160 19170 19180 19190 19200
SVGXLYSGCR LTXLRXEKXG AATGXDAICX HXXXPKXPGL XXEXLYWELS XLTXXIXELG
          19210 19220 19230 19240 19250 19260
PYTLDRXSLY VNGFTHWSSG LTTSTPWTST VDLGTSGTPS PVPSPTTAGP LLVPFTLNFT
          19270 19280 19290 19300 19310 19320
ITNLQYEEDM HRPGSRKFNA TERVLQGLLS PIFKNTSVGP LYSGCRLTLL RPEKQEAATG
          19330 19340 19350 19360 19370 19380
VDTICTHRVD PIGPGLXXEX LYWELSXLTX XIXELGPYTL DRXSLYVNGF THXXSXPTTS
          19390 19400 19410 19420 19430 19440
TPGTSTVXXG TSGTPSSXPX XTXXXPLLXP FTXNXTITNL XXXXXMXXPG SRKFNTTEXV
          19450 19460 19470 19480 19490 19500
LQGLLXPXFK NXSVGXLYSG CRLTXLRXEK XGAATGXDAI CXHXXXPKXP GLXXEXLYWE
          19510 19520 19530 19540 19550 19560
LSXLTXXIXE LGPYTLDRXS LYVNGFTHRS FGLTTSTPWT STVDLGTSGT PSPVPSPTTA
          19570 19580 19590 19600 19610 19620
GPLLVPFTLN FTITNLQYEE DMHRPGSRKF NTTERVLQGL LTPLFRNTSV SSLYSGCRLT
          19630 19640 19650 19660 19670 19680
LLRPEKDGAA TRVDAVCTHR PDPKSPGLXX EXLYWELSXL TXXIXELGPY TLDRXSLYVN
          19690 19700 19710 19720 19730 19740
GFTHXXSXPT TSTPGTSTVX XGTSGTPSSX PXXTXXXPLL XPFTXNXTIT NLXXXXXMXX
          19750 19760 19770 19780 19790 19800
PGSRKFNTTE XVLQGLLXPX FKNXSVGXLY SGCRLTXLRX EKXGAATGXD AICXHXXXPK
          19810 19820 19830 19840 19850 19860
XPGLXXEXLY WELSXLTXXI XELGPYTLDR XSLYVNGFTH WIPVPTSSTP GTSTVDLGSG
          19870 19880 19890 19900 19910 19920
TPSSLPSPTT AGPLLVPFTL NFTITNLQYG EDMGHPGSRK FNTTERVLQG LLGPIFKNTS
          19930 19940 19950 19960 19970 19980
VGPLYSGCRL TSLRSEKDGA ATGVDAICIH HLDPKSPGLX XEXLYWELSX LTXXIXELGP
          19990 20000 20010 20020 20030 20040
YTLDRXSLYV NGFTHXXSXP TTSTPGTSTV XXGTSGTPSS XPXXTXXXPL LXPFTXNXTI.
          20050 20060 20070 20080 20090 20100
TNLXXXXXMX XPGSRKFNTT EXVLQGLLXP XFKNXSVGXL YSGCRLTXLR XEKXGAATGX
          20110 20120 20130 20140 20150 20160
DAICXHXXXP KXPGLXXEXL YWELSXLTXX IXELGPYTLD RXSLYVNGFT HQTFAPNTST
          20170 20180 20190 20200 20210 20220
PGTSTVDLGT SGTPSSLPSP TSAGPLLVPF TLNFTITNLQ YEEDMHHPGS RKFNTTERVL
          20230 20240 20250 20260 20270 20280
QGLLGPMFKN TSVGLLYSGC RLTLLRPEKN GAATRVDAVC THRPDPKSPG LXXEXLYWEL
          20290 20300 20310 20320 20330 20340
SXLTXXIXEL GPYTLDRXSL YVNGFTHXXS XPTTSTPGTS TVXXGTSGTP SSXPXXTAPV
          20350 20360 20370 20380 20390 20400
PLLIPFTLNF TITNLHYEEN MQHPGSRKFN TTERVLQGLL KPLFKSTSVG PLYSGCRLTL
          20410 20420 20430 20440 20450 20460
LRPEKHGAAT GVDAICTLRL DPTGPGLDRE RLYWELSQLT NSVTELGPYT LDRDSLYVNG
          20470 20480 20490 20500 20510 20520
FTQRSSVPTT SIPGTSAVHL ETSGTPASLP GHTAPGPLLV PFTLNFTITN LQYEVDMRHP
          20530 20540 20550 20560 20570 20580
GSRKFNTTER VLQGLLKPLF KSTSVGPLYS GCRLTLLRPE KRGAATGVDT ICTHRLDPLN
          20590 20600 20610 20620 20630 20640
PGLDREQLYW ELSKLTRGII ELGPYLLDRG SLYVNGFTHR NFVPITSTPG TSTVHLGTSE
          20650 20660 20670 20680 20690 20700
TPSSLPRPIV PGPLLVPFTL NFTITNLQYE EAMRHPGSRK FNTTERVLQG LLRPLFKNTS
          20710 20720 20730 20740 20750 20760
IGPLYSSCRL TLLRPEKDKA ATRVDAICTH HPDPQSPGLN REQLYWELSQ LTHGITELGP
          20770 20780 20790 20800 20810 20820
YTLDRDSLYV DGFTHWSPIP TTSTPGTSIV NLGTSGIPPS LPETTXXXPL LXPFTXNXTI
          20830 20840 20850 20860 20870 20880
TNLXXXXXMX XPGSRKFNTT ERVLQGLLKP LFKSTSVGPL YSGCRLTLLR PEKDGVATRV
          20890 20900 20910 20920 20930 20940
DAICTHRPDP KIPGLDRQQL YWELSQLTHS ITELGPYTLD RDSLYVNGFT QRSSVPTTST
          20950 20960 20970 20980 20990 21000
PGTFTVQPET SETPSSLPGP TATGPVLLPF TLNFTITNLQ YEEDMHRPGS RKFNTTERVL
          21010 21020 21030 21040 21050 21060
QGLLMPLFKN TSVSSLYSGC RLTLLRPEKD GAATRVDAVC THRPDPKSPG LDRERLYWKL
          21070 21080 21090 21100 21110 21120
SQLTHGITEL GPYTLDRHSL YVNGFTHQSS MTTTRTPDTS TMHLATSRTP ASLSGPTTAS
          21130 21140 21150 21160 21170 21180
PLLVLFTINF TITNLRYEEN MHHPGSRKFN TTERVLQGLL RPVFKNTSVG PLYSGCRLTL
          21190 21200 21210 21220 21230 21240
LRPKKDGAAT KVDAICTYRP DPKSPGLDRE QLYWELSQLT HSITELGPYT LDRDSLYVNG
          21250 21260 21270 21280 21290 21300
FTQRSSVPTT SIPGTPTVDL GTSGTPVSKP GPSAASPLLV LFTLNFTITN LRYEENMQHP
          21310 21320 21330 21340 21350 21360
GSRKFNTTER VLQGLLRSLF KSTSVGPLYS GCRLTLLRPE KDGTATGVDA ICTHHPDPKS
          21370 21380 21390 21400 21410 21420
PRLDREQLYW ELSQLTHNIT ELGHYALDND SLFVNGFTHR SSVSTTSTPG TPTVYLGASK
          21430 21440 21450 21460 21470 21480
TPASIFGPSA ASHLLILFTL NFTITNLRYE ENMWPGSRKF NTTERVLQGL LRPLFKNTSV
          21490 21500 21510 21520 21530 21540
GPLYSGSRLT LLRPEKDGEA TGVDAICTHR PDPTGPGLDR EQLYLELSQL THSITELGPY
          21550 21560 21570 21580 21590 21600
TLDRDSLYVN GFTHRSSVPT TSTGVVSEEP FTLNFTINNL RYMADMGQPG SLKFNITDNV
          21610 21620 21630 21640 21650 21660
MKHLLSPLFQ RSSLGARYTG CRVIALRSVK NGAETRVDLL CTYLQPLSGP GLPIKQVFHE
          21670 21680 21690 21700 21710 21720
LSQQTHGITR LGPYSLDKDS LYLNGYNEPG LDEPPTTPKP ATTFLPPLSE ATTAMGYHLK
          21730 21740 21750 21760 21770 21780
TLTLNFTISN LQYSPDMGKG SATFNSTEGV LQHLLRPLFQ KSSMGPFYLG CQLISLRPEK
          21790 21800 21810 21820 21830 21840
DGAATGVDTT CTYHPDPVGP GLDIQQLYWE LSQLTHGVTQ LGFYVLDRDS LFINGYAPQN
          21850 21860 21870 21880 21890 21900
LSIRGEYQIN FHIVNWNLSN PDPTSSEYIT LLRDIQDKVT TLYKGSQLHD TFRFCLVTNL
          21910 21920 21930 21940 21950 21960
TMDSVLVTVK ALFSSNLDPS LVEQVFLDKT LNASFHWLGS TYQLVDIHVT EMESSVYQPT
          21970 21980 21990 22000 22010 22020
SSSSTQHFYL NFTITNLPYS QDKAQPGTTN YQRNKRNIED ALNQLFRNSS IKSYFSDCQV
          22030 22040 22050 22060 22070 22080
STFRSVPNRH HTGVDSLCNF SPLARRVDRV AIYEEFLRMT RNGTQLQNFT LDRSSVLVDG
          22090 22100 22110 22120 22130 22140
YSPNRNEPLT GNSDLPFWAV ILIGLAGLLG LITCLICGVL VTTRRRKKEG EYNVQQQCPG
          22150
YYQSHLDLED LQ
```

Cancer antigen CA 15-3 is a single-pass type I membrane protein having a large extracellular domain, some or all of which is present in soluble forms Cancer antigen CA 15-3 generated either through alternative splicing event which deletes all or a portion of the transmembrane domain, or by proteolysis of the membrane-bound form. In the case of an immunoassay, one or more antibodies that bind to epitopes within this extracellular domain may be used to detect these soluble form(s). The following domains have been identified in Cancer antigen CA 15-3:

| Residues | Length | Domain ID |
|---|---|---|
| 1-22152 | 22152 | Cancer antigen CA 15-3 |
| 1-22096 | 22096 | Extracellular domain |
| 22097-22117 | 21 | Transmembrane domain |
| 22118-22152 | 35 | Cytoplasmic domain |

As used herein, the term “C-C motif chemokine 18” refers to one or more polypeptides present in a biological sample that are derived from the C-C motif chemokine 18 precursor (Swiss-Prot P55774 (SEQ ID NO: 8)).

```
                10 20 30 40 50 60
MKGLAAALLV LVCTMALCSC AQVGTNKELC CLVYTSWQIP QKFIVDYSET SPQCPKPGVI
                70 80
LLTKRGRQIC ADPNKKWVQK YISDLKLNA
```

The following domains have been identified in C-C motif chemokine 18:

| Residues | Length | Domain ID |
|---|---|---|
| 1-20 | 20 | Signal peptide |
| 21-89 | 69 | C-C motif chemokine 18 |
| 21-88 | 68 | CCL 18 (1-68) |
| 23-89 | 67 | CCL 18 (3-69) |
| 24-89 | 66 | CCL 18 (4-69) |

As used herein, the term “C-C motif chemokine 24” refers to one or more polypeptides present in a biological sample that are derived from the C-C motif chemokine 24 precursor (Swiss-Prot O00175 (SEQ ID NO: 9)).

```
                10 20 30 40 50 60
MAGLMTIVTS LLFLGVCAHH IIPTGSVVIP SPCCMFFVSK RIPENRVVSY QLSSRSTCLK
                70 80 90 100 110
AGVIFTTKKG QQFCGDPKQE WVQRYMKNLD AKQKKASPRA RAVAVKGPVQ RYPGNQTTC
```

The following domains have been identified in C-C motif chemokine 24:

| Residues | Length | Domain ID |
|---|---|---|
| 1-26 | 26 | Signal peptide |
| 27-119 | 93 | C-C motif chemokine 24 |

As used herein, the term “Cathepsin D” refers to one or more polypeptides present in a biological sample that are derived from the Cathepsin D precursor (Swiss-Prot P07339 (SEQ ID NO: 10)).

```
                10 20 30 40 50 60
MQPSSLLPLA LCLLAAPASA LVRIPLHKFT SIRRTMSEVG GSVEDLIAKG PVSKYSQAVP
                70 80 90 100 110 120
AVTEGPIPEV LKNYMDAQYY GEIGIGTPPQ CFTVVFDTGS SNLWVPSIHC KLLDIACWIH
              130 140 150 160 170 180
HKYNSDKSST YVKNGTSFDI HYGSGSLSGY LSQDTVSVPC QSASSASALG GVKVERQVFG
              190 200 210 220 230 240
EATKQPGITF IAAKFDGILG MAYPRISVNN VLPVFDNLMQ QKLVDQNIFS FYLSRDPDAQ
              250 260 270 280 290 300
PGGELMLGGT DSKYYKGSLS YLNVTRKAYW QVHLDQVEVA SGLTLCKEGC EAIVDTGTSL
              310 320 330 340 350 360
MVGPVDEVRE LQKAIGAVPL IQGEYMIPCE KVSTLPAITL KLGGKGYKLS PEDYTLKVSQ
              370 380 390 400 410
AGKTLCLSGF MGMDIPPPSG PLWILGDVFI GRYYTVFDRD NNRVGFAEAA RL
```

The following domains have been identified in Capthesin D:

| Residues | Length | Domain ID |
|---|---|---|
| 1-18 | 18 | Signal peptide |
| 19-64 | 46 | Activation peptide |
| 65-412 | 348 | Cathepsin D |
| 65-161 | 348 | Cathepsin D light chain |
| 169-412 | 348 | Cathepsin D heavy chain |

As used herein, the term “C-X-C Motif chemokine 13” refers to one or more polypeptides present in a biological sample that are derived from the C-X-C Motif chemokine 13 precursor (Swiss-Prot O43927 (SEQ ID NO: 11)).

```
                10 20 30 40 50 60
MKFISTSLLL MLLVSSLSPV QGVLEVYYTS LRCRCVQESS VFIPRRFIDR IQILPRGNGC
                70 80 90 100
PRKEIIVWKK NKSIVCVDPQ AEWIQRMMEV LRKRSSSTLP VPVFKRKIP
```

The following domains have been identified in C-X-C Motif chemokine 13:

| Residues | Length | Domain ID |
|---|---|---|
| 1-22 | 22 | Signal peptide |
| 23-109 | 87 | C-X-C Motif chemokine 13 |

As used herein, the term “IgG1” refers to subclass 1 of the glycoprotein immunoglobulin G (IgG), a major effector molecule of the humoral immune response in man. Antibodies of the IgG class express their predominant activity during a secondary antibody response. The basic immunoglobulin G molecule has a four-chain structure, comprising two identical heavy (H) chains and two identical light (L) chains, linked together by inter-chain disulfide bonds. Each heavy chain is encoded by 4 distinct types of gene segments, designated V_{H} (variable), D (diversity), J_{H} (joining) and C_{H}(constant). The variable region of the heavy chain is encoded by the V_{H}, D and J_{H} segments. The light chains are encoded by the 3 gene segments, V_{L}, J_{L} and C_{L}. The variable region of the light chains is encoded by the V_{L} and J_{L} segments.

As used herein, the term “IgG2” refers to subclass 2 of the glycoprotein immunoglobulin G (IgG), a major effector molecule of the humoral immune response in man. Antibodies of the IgG class express their predominant activity during a secondary antibody response. The basic immunoglobulin G molecule has a four-chain structure, comprising two identical heavy (H) chains and two identical light (L) chains, linked together by inter-chain disulfide bonds. Each heavy chain is encoded by 4 distinct types of gene segments, designated V_{H} (variable), D (diversity), J_{H} (joining) and C_{H}(constant). The variable region of the heavy chain is encoded by the V_{H}, D and J_{H} segments. The light chains are encoded by the 3 gene segments, V_{L}, J_{L} and C_{L}. The variable region of the light chains is encoded by the V_{L} and J_{L} segments.

The length and flexibility of the hinge region varies among the IgG subclasses. The hinge region of IgG1 encompasses amino acids 216-231 and since it is freely flexible, the Fab fragments can rotate about their axes of symmetry and move within a sphere centered at the first of two inter-heavy chain disulfide bridges (23). IgG2 has a shorter hinge than IgG1, with 12 amino acid residues and four disulfide bridges. The hinge region of IgG2 lacks a glycine residue, it is relatively short and contains a rigid poly-proline double helix, stabilised by extra inter-heavy chain disulfide bridges. These properties restrict the flexibility of the IgG2 molecule (24). IgG3 differs from the other subclasses by its unique extended hinge region (about four times as long as the IgG1 hinge), containing 62 amino acids (including 21 prolines and 11 cysteines), forming an inflexible poly-proline double helix (25,26). In IgG3 the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2.

The four IgG subclasses also differ with respect to the number of inter-heavy chain disulfide bonds in the hinge region (26). The structural differences between the IgG subclasses are also reflected in their susceptibility to proteolytic enzymes. IgG3 is very susceptible to cleavage by these enzymes, whereas IgG2 is relatively resistant. IgG1 and IgG4 exhibit an intermediary sensitivity, depending upon the enzyme used. Since these proteolytic enzymes all cleave IgG molecules near or within the hinge region, it is likely that the high sensitivity of IgG3 to enzyme digestion is related to its accessible hinge. Another structural difference between the human IgG subclasses is the linkage of the heavy and light chain by a disulfide bond. This bond links the carboxy-terminal of the light chain with the cysteine residue at position 220 (in IgG) or at position 131 (in IgG2, IgG3 and IgG4) of the CH1 sequence of the heavy chain.

As a consequence of the structural differences, the four IgG subclasses may be distinguished from one another, for example using antibodies that are specific for differences between the isoforms. In the present application, a level of IgG1 is determined using an assay which distinguishes this subclass, relative to the other subclasses.

As used herein, the term “relating a signal to the presence or amount” of an analyte reflects the following understanding. Assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Because an antibody epitope is on the order of 8 amino acids, an immunoassay configured to detect a marker of interest will also detect polypeptides related to the marker sequence, so long as those polypeptides contain the epitope(s) necessary to bind to the antibody or antibodies used in the assay. The term “related marker” as used herein with regard to a biomarker such as one of the kidney injury markers described herein refers to one or more fragments, variants, etc., of a particular marker or its biosynthetic parent that may be detected as a surrogate for the marker itself or as independent biomarkers. The term also refers to one or more polypeptides present in a biological sample that are derived from the biomarker precursor complexed to additional species, such as binding proteins, receptors, heparin, lipids, sugars, etc.

In this regard, the skilled artisan will understand that the signals obtained from an immunoassay are a direct result of complexes formed between one or more antibodies and the target biomolecule (i.e., the analyte) and polypeptides containing the necessary epitope(s) to which the antibodies bind. While such assays may detect the full length biomarker and the assay result be expressed as a concentration of a biomarker of interest, the signal from the assay is actually a result of all such “immunoreactive” polypeptides present in the sample. Expression of biomarkers may also be determined by means other than immunoassays, including protein measurements (such as dot blots, western blots, chromatographic methods, mass spectrometry, etc.) and nucleic acid measurements (mRNA quatitation). This list is not meant to be limiting.

The term “positive going” marker as that term is used herein refer to a marker that is determined to be elevated in subjects suffering from a disease or, condition, relative to subjects not suffering from that disease or condition. The term “negative going” marker as that term is used herein refer to a marker that is determined to be reduced in subjects suffering from a disease or condition, relative to subjects not suffering from that disease or condition.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

Preferably, an analyte is measured in a sample. Such a sample may be obtained from a subject, or may be obtained from biological materials intended to be provided to the subject. For example, a sample may be obtained from a kidney being evaluated for possible transplantation into a subject, and an analyte measurement used to evaluate the kidney for preexisting damage. Preferred samples are body fluid samples.

The term “body fluid sample” as used herein refers to a sample of bodily fluid obtained for the purpose of diagnosis, prognosis, classification or evaluation of a subject of interest, such as a patient or transplant donor. In certain embodiments, such a sample may be obtained for the purpose of determining the outcome of an ongoing condition or the effect of a treatment regimen on a condition. Preferred body fluid samples include blood, serum, plasma, cerebrospinal fluid, urine, saliva, sputum, and pleural effusions. In addition, one of skill in the art would realize that certain body fluid samples would be more readily analyzed following a fractionation or purification procedure, for example, separation of whole blood into serum or plasma components.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, for a kidney injury marker of the present invention, optionally together with other clinical characteristics, to arrive at a diagnosis (that is, the occurrence or nonoccurrence) of an acute renal injury or ARF for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening renal function, future ARF, or death) is referred to as being “indicative of an increased likelihood” of an adverse outcome in a patient.

Marker Assays

In general, immunoassays involve contacting a sample containing or suspected of containing a biomarker of interest with at least one antibody that specifically binds to the biomarker. A signal is then generated indicative of the presence or amount of complexes formed by the binding of polypeptides in the sample to the antibody. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, figures and claims.

The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing immunoassays. But any suitable immunoassay may be utilized, for example, enzyme-linked immunoassays (ELISA), radioimmunoassays (RIAs), competitive binding assays, and the like.

Antibodies or other polypeptides may be immobilized onto a variety of solid supports for use in assays. Solid phases that may be used to immobilize specific binding members include include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the later case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

In certain aspects, the present invention provides kits for the analysis of the described kidney injury markers. The kit comprises reagents for the analysis of at least one test sample which comprise at least one antibody that a kidney injury marker. The kit can also include devices and instructions for performing one or more of the diagnostic and/or prognostic correlations described herein. Preferred kits will comprise an antibody pair for performing a sandwich assay, or a labeled species for performing a competitive assay, for the analyte. Preferably, an antibody pair comprises a first antibody conjugated to a solid phase and a second antibody conjugated to a detectable label, wherein each of the first and second antibodies that bind a kidney injury marker. Most preferably each of the antibodies are monoclonal antibodies. The instructions for use of the kit and performing the correlations can be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

Antibodies

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Antibodies used in the immunoassays described herein preferably specifically bind to a kidney injury marker of the present invention. The term “specifically binds” is not intended to indicate that an antibody binds exclusively to its intended target since, as noted above, an antibody binds to any polypeptide displaying the epitope(s) to which the antibody binds. Rather, an antibody “specifically binds” if its affinity for its intended target is about 5-fold greater when compared to its affinity for a non-target molecule which does not display the appropriate epitope(s). Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Numerous publications discuss the use of phage display technology to produce and screen libraries of polypeptides for binding to a selected analyte. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

While the present application describes antibody-based binding assays in detail, alternatives to antibodies as binding species in assays are well known in the art. These include receptors for a particular target, aptamers, etc. Aptamers are oligonucleic acid or peptide molecules that bind to a specific target molecule. Aptamers are usually created by selecting them from a large random sequence pool, but natural aptamers also exist. High-affinity aptamers containing modified nucleotides conferring improved characteristics on the ligand, such as improved in vivo stability or improved delivery characteristics. Examples of such modifications include chemical substitutions at the ribose and/or phosphate and/or base positions, and may include amino acid side chain functionalities.

Assay Correlations

The term “correlating” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy which is highly efficacious and has a low level of risk, few tests are needed because clinicians can accept substantial diagnostic uncertainty. On the other hand, in situations where treatment options are less effective and more risky, clinicians often need a higher degree of diagnostic certainty. Thus, cost/benefit analysis is involved in selecting a diagnostic threshold.

Suitable thresholds may be determined in a variety of ways. For example, one recommended diagnostic threshold for the diagnosis of acute myocardial infarction using cardiac troponin is the 97.5th percentile of the concentration seen in a normal population. Another method may be to look at serial samples from the same patient, where a prior “baseline” result is used to monitor for temporal changes in a biomarker level.

Population studies may also be used to select a decision threshold. Reciever Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when the person tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1-specificity, the ROC graph is sometimes called the sensitivity vs (1-specificity) plot. A perfect test will have an area under the ROC curve of 1.0; a random test will have an area of 0.5. A threshold is selected to provide an acceptable level of specificity and sensitivity.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.8, even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1

Additional clinical indicia may be combined with the kidney injury marker assay result(s) of the present invention. These include other biomarkers related to renal status. Examples include the following, which recite the common biomarker name, followed by the Swiss-Prot entry number for that biomarker or its parent: Actin (P68133); Adenosine deaminase binding protein (DPP4, P27487); Alpha-1-acid glycoprotein 1 (P02763); Alpha-1-microglobulin (P02760); Albumin (P02768); Angiotensinogenase (Renin, P00797); Annexin A2 (P07355); Beta-glucuronidase (P08236); B-2-microglobulin (P61679); Beta-galactosidase (P16278); BMP-7 (P18075); Brain natriuretic peptide (proBNP, BNP-32, NTproBNP; P16860); Calcium-binding protein Beta (S100-beta, P04271); Carbonic anhydrase (Q16790); Casein Kinase 2 (P68400); Ceruloplasmin (P00450); Clusterin (P10909); Complement C3 (P01024); Cysteine-rich protein (CYR61, O00622); Cytochrome C(P99999); Epidermal growth factor (EGF, P01133); Endothelin-1 (P05305); Exosomal Fetuin-A (P02765); Fatty acid-binding protein, heart (FABP3, P05413); Fatty acid-binding protein, liver (P07148); Ferritin (light chain, P02793; heavy chain P02794); Fructose-1,6-biphosphatase (P09467); GRO-alpha (CXCL1, (P09341); Growth Hormone (P01241); Hepatocyte growth factor (P14210); Insulin-like growth factor I (P01343); Immunoglobulin G; Immunoglobulin Light Chains (Kappa and Lambda); Interferon gamma (P01308); Lysozyme (P61626); Interleukin-1alpha (P01583); Interleukin-2 (P60568); Interleukin-4 (P60568); Interleukin-9 (P15248); Interleukin-12p40 (P29460); Interleukin-13 (P35225); Interleukin-16 (Q14005); L1 cell adhesion molecule (P32004); Lactate dehydrogenase (P00338); Leucine Aminopeptidase (P28838); Meprin A-alpha subunit (Q16819); Meprin A-beta subunit (Q16820); Midkine (P21741); MIP2-alpha (CXCL2, P19875); MMP-2 (P08253); MMP-9 (P14780); Netrin-1 (O95631); Neutral endopeptidase (P08473); Osteopontin (P10451); Renal papillary antigen 1 (RPA1); Renal papillary antigen 2 (RPA2); Retinol binding protein (P09455); Ribonuclease; S100 calcium-binding protein A6 (P06703); Serum Amyloid P Component (P02743); Sodium/Hydrogen exchanger isoform (NHE3, P48764); Spermidine/spermine N1-acetyltransferase (P21673); TGF-Beta1 (P01137); Transferrin (P02787); Trefoil factor 3 (TFF3, Q07654); Toll-Like protein 4 (O00206); Total protein; Tubulointerstitial nephritis antigen (Q9UJW2); Uromodulin (Tamm-Horsfall protein, P07911).

For purposes of risk stratification, Adiponectin (Q15848); Alkaline phosphatase (P05186); Aminopeptidase N(P15144); CalbindinD28k (P05937); Cystatin C(P01034); 8 subunit of FIFO ATPase (P03928); Gamma-glutamyltransferase (P19440); GSTa (alpha-glutathione-S-transferase, P08263); GSTpi (Glutathione-S-transferase P; GST class-pi; P09211); IGFBP-1 (P08833); IGFBP-2 (P18065); IGFBP-6 (P24592); Integral membrane protein 1 (Itml, P46977); Interleukin-6 (P05231); Interleukin-8 (P10145); Interleukin-18 (Q14116); IP-10 (10 kDa interferon-gamma-induced protein, P02778); IRPR (IFRD1, O00458); Isovaleryl-CoA dehydrogenase (IVD, P26440); I-TAC/CXCL11 (O14625); Keratin 19 (P08727); Kim-1 (Hepatitis A virus cellular receptor 1, O43656); L-arginine:glycine amidinotransferase (P50440); Leptin (P41159); Lipocalin2 (NGAL, P80188); MCP-1 (P13500); MIG (Gamma-interferon-induced monokine Q07325); MIP-1a (P10147); MIP-3a (P78556); MIP-1beta (P13236); MIP-1d (Q16663); NAG (N-acetyl-beta-D-glucosaminidase, P54802); Organic ion transporter (OCT2, O15244); Osteoprotegerin (O14788); P8 protein (O60356); Plasminogen activator inhibitor 1 (PAI-1, P05121); ProANP(1-98) (P01160); Protein phosphatase 1-beta (PPI-beta, P62140); Rab GDI-beta (P50395); Renal kallikrein (Q86U61); RT1.B-1 (alpha) chain of the integral membrane protein (Q5Y7A8); Soluble tumor necrosis factor receptor superfamily member 1A (sTNFR-I, P19438); Soluble tumor necrosis factor receptor superfamily member 1B (sTNFR-11, P20333); Tissue inhibitor of metalloproteinases 3 (TIMP-3, P35625); uPAR (Q03405) may be combined with the kidney injury marker assay result(s) of the present invention.

Other clinical indicia which may be combined with the kidney injury marker assay result(s) of the present invention includes demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score), a urine total protein measurement, a glomerular filtration rate, an estimated glomerular filtration rate, a urine production rate, a serum or plasma creatinine concentration, a renal papillary antigen 1 (RPA1) measurement; a renal papillary antigen 2 (RPA2) measurement; a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatnine ratio, and/or a renal failure index calculated as urine sodium/(urine creatinine/plasma creatinine). Other measures of renal function which may be combined with the kidney injury marker assay result(s) are described hereinafter and in Harrison's Principles of Internal Medicine, 17^{th} Ed., McGraw Hill, New York, pages 1741-1830, and Current Medical Diagnosis & Treatment 2008, 47^{th} Ed, McGraw Hill, New York, pages 785-815, each of which are hereby incorporated by reference in their entirety.

Combining assay results/clinical indicia in this manner can comprise the use of multivariate logistical regression, loglinear modeling, neural network analysis, n-of-m analysis, decision tree analysis, etc. This list is not meant to be limiting.

Diagnosis of Acute Renal Failure

As noted above, the terms “acute renal (or kidney) injury” and “acute renal (or kidney) failure” as used herein are defined in part in terms of changes in serum creatinine from a baseline value. Most definitions of ARF have common elements, including the use of serum creatinine and, often, urine output. Patients may present with renal dysfunction without an available baseline measure of renal function for use in this comparison. In such an event, one may estimate a baseline serum creatinine value by assuming the patient initially had a normal GFR. Glomerular filtration rate (GFR) is the volume of fluid filtered from the renal (kidney) glomerular capillaries into the Bowman's capsule per unit time. Glomerular filtration rate (GFR) can be calculated by measuring any chemical that has a steady level in the blood, and is freely filtered but neither reabsorbed nor secreted by the kidneys. GFR is typically expressed in units of ml/min:

$$G F R = \frac{Urine Concentration \times Urine Flow}{Plasma Concentration}$$

By normalizing the GFR to the body surface area, a GFR of approximately 75-100 ml/min per 1.73 m² can be assumed. The rate therefore measured is the quantity of the substance in the urine that originated from a calculable volume of blood.

There are several different techniques used to calculate or estimate the glomerular filtration rate (GFR or eGFR). In clinical practice, however, creatinine clearance is used to measure GFR. Creatinine is produced naturally by the body (creatinine is a metabolite of creatine, which is found in muscle). It is freely filtered by the glomerulus, but also actively secreted by the renal tubules in very small amounts such that creatinine clearance overestimates actual GFR by 10-20%. This margin of error is acceptable considering the ease with which creatinine clearance is measured.

Creatinine clearance (CCr) can be calculated if values for creatinine's urine concentration (U_{Cr}), urine flow rate (V), and creatinine's plasma concentration (P_{Cr}) are known. Since the product of urine concentration and urine flow rate yields creatinine's excretion rate, creatinine clearance is also said to be its excretion rate (U_{Cr}×V) divided by its plasma concentration. This is commonly represented mathematically as:

$${C}_{Cr} = \frac{{U}_{Cr} \times V}{{P}_{Cr}}$$

Commonly a 24 hour urine collection is undertaken, from empty-bladder one morning to the contents of the bladder the following morning, with a comparative blood test then taken:

$${C}_{Cr} = \frac{{U}_{Cr} \times 24 \text{-} hour volume}{{P}_{cr} \times 24 \times 60 mins}$$

To allow comparison of results between people of different sizes, the CCr is often corrected for the body surface area (BSA) and expressed compared to the average sized man as ml/min/1.73 m2. While most adults have a BSA that approaches 1.7 (1.6-1.9), extremely obese or slim patients should have their CCr corrected for their actual BSA:

$${C}_{Cr \text{-} corrected} = \frac{{C}_{Cr} \times 1.73}{B S A}$$

The accuracy of a creatinine clearance measurement (even when collection is complete) is limited because as glomerular filtration rate (GFR) falls creatinine secretion is increased, and thus the rise in serum creatinine is less. Thus, creatinine excretion is much greater than the filtered load, resulting in a potentially large overestimation of the GFR (as much as a twofold difference). However, for clinical purposes it is important to determine whether renal function is stable or getting worse or better. This is often determined by monitoring serum creatinine alone. Like creatinine clearance, the serum creatinine will not be an accurate reflection of GFR in the non-steady-state condition of ARF. Nonetheless, the degree to which serum creatinine changes from baseline will reflect the change in GFR. Serum creatinine is readily and easily measured and it is specific for renal function.

For purposes of determining urine output on a Urine output on a mL/kg/hr basis, hourly urine collection and measurement is adequate. In the case where, for example, only a cumulative 24-h output was available and no patient weights are provided, minor modifications of the RIFLE urine output criteria have been described. For example, Bagshaw et al., Nephrol. Dial. Transplant. 23: 1203-1210, 2008, assumes an average patient weight of 70 kg, and patients are assigned a RIFLE classification based on the following: <35 mL/h (Risk), <21 mL/h (Injury) or <4 mL/h (Failure).

Selecting a Treatment Regimen

Once a diagnosis is obtained, the clinician can readily select a treatment regimen that is compatible with the diagnosis, such as initiating renal replacement therapy, withdrawing delivery of compounds that are known to be damaging to the kidney, kidney transplantation, delaying or avoiding procedures that are known to be damaging to the kidney, modifying diuretic administration, initiating goal directed therapy, etc. The skilled artisan is aware of appropriate treatments for numerous diseases discussed in relation to the methods of diagnosis described herein. See, e.g., Merck Manual of Diagnosis and Therapy, 17th Ed. Merck Research Laboratories, Whitehouse Station, N.J., 1999. In addition, since the methods and compositions described herein provide prognostic information, the markers of the present invention may be used to monitor a course of treatment. For example, improved or worsened prognostic state may indicate that a particular treatment is or is not efficacious.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### Example 1

### Contrast-Induced Nephropathy Sample Collection

The objective of this sample collection study is to collect samples of plasma and urine and clinical data from patients before and after receiving intravascular contrast media. Approximately 250 adults undergoing radiographic/angiographic procedures involving intravascular administration of iodinated contrast media are enrolled. To be enrolled in the study, each patient must meet all of the following inclusion criteria and none of the following exclusion criteria:

### Inclusion Criteria

males and females 18 years of age or older;
undergoing a radiographic/angiographic procedure (such as a CT scan or coronary intervention) involving the intravascular administration of contrast media;
expected to be hospitalized for at least 48 hours after contrast administration.
able and willing to provide written informed consent for study participation and to comply with all study procedures.

### Exclusion Criteria

renal transplant recipients;
acutely worsening renal function prior to the contrast procedure;
already receiving dialysis (either acute or chronic) or in imminent need of dialysis at enrollment;
expected to undergo a major surgical procedure (such as involving cardiopulmonary bypass) or an additional imaging procedure with contrast media with significant risk for further renal insult within the 48 hrs following contrast administration;
participation in an interventional clinical study with an experimental therapy within the previous 30 days;
known infection with human immunodeficiency virus (HIV) or a hepatitis virus.

Immediately prior to the first contrast administration (and after any pre-procedure hydration), an EDTA anti-coagulated blood sample (10 mL) and a urine sample (10 mL) are collected from each patient. Blood and urine samples are then collected at 4 (±0.5), 8 (±1), 24 (±2) 48 (±2), and 72 (±2) hrs following the last administration of contrast media during the index contrast procedure. Blood is collected via direct venipuncture or via other available venous access, such as an existing femoral sheath, central venous line, peripheral intravenous line or hep-lock. These study blood samples are processed to plasma at the clinical site, frozen and shipped to Astute Medical, Inc., San Diego, Calif. The study urine samples are frozen and shipped to Astute Medical, Inc.

Serum creatinine is assessed at the site immediately prior to the first contrast administration (after any pre-procedure hydration) and at 4 (±0.5), 8 (±1), 24 (±2) and 48 (±2)), and 72 (±2) hours following the last administration of contrast (ideally at the same time as the study samples are obtained). In addition, each patient's status is evaluated through day 30 with regard to additional serum and urine creatinine measurements, a need for dialysis, hospitalization status, and adverse clinical outcomes (including mortality).

Prior to contrast administration, each patient is assigned a risk based on the following assessment: systolic blood pressure <80 mm Hg=5 points; intra-arterial balloon pump=5 points; congestive heart failure (Class III-IV or history of pulmonary edema)=5 points; age>75 yrs=4 points; hematocrit level<39% for men, <35% for women=3 points; diabetes=3 points; contrast media volume=1 point for each 100 mL; serum creatinine level>1.5 g/dL=4 points OR estimated GFR 40-60 mL/min/1.73 m²=2 points, 20-40 mL/min/1.73 m²=4 points, <20 mL/min/1.73 m²=6 points. The risks assigned are as follows: risk for CIN and dialysis: 5 or less total points=risk of CIN−7.5%, risk of dialysis−0.04%; 6-10 total points=risk of CIN−14%, risk of dialysis−0.12%; 11-16 total points=risk of CIN−26.1%, risk of dialysis−1.09%; >16 total points=risk of CIN−57.3%, risk of dialysis−12.8%.

### Example 2

### Cardiac Surgery Sample Collection

The objective of this sample collection study is to collect samples of plasma and urine and clinical data from patients before and after undergoing cardiovascular surgery, a procedure known to be potentially damaging to kidney function. Approximately 900 adults undergoing such surgery are enrolled. To be enrolled in the study, each patient must meet all of the following inclusion criteria and none of the following exclusion criteria:

### Inclusion Criteria

males and females 18 years of age or older;
undergoing cardiovascular surgery;
Toronto/Ottawa Predictive Risk Index for Renal Replacement risk score of at least 2 (Wijeysundera et al., JAMA 297: 1801-9, 2007); and
able and willing to provide written informed consent for study participation and to comply with all study procedures.

### Exclusion Criteria

known pregnancy;
previous renal transplantation;
acutely worsening renal function prior to enrollment (e.g., any category of RIFLE criteria);
already receiving dialysis (either acute or chronic) or in imminent need of dialysis at enrollment;
currently enrolled in another clinical study or expected to be enrolled in another clinical study within 7 days of cardiac surgery that involves drug infusion or a therapeutic intervention for AKI;
known infection with human immunodeficiency virus (HIV) or a hepatitis virus.

Within 3 hours prior to the first incision (and after any pre-procedure hydration), an EDTA anti-coagulated blood sample (10 mL), whole blood (3 mL), and a urine sample (35 mL) are collected from each patient. Blood and urine samples are then collected at 3 (±0.5), 6 (±0.5), 12 (±1), 24 (±2) and 48 (±2) hrs following the procedure and then daily on days 3 through 7 if the subject remains in the hospital. Blood is collected via direct venipuncture or via other available venous access, such as an existing femoral sheath, central venous line, peripheral intravenous line or hep-lock. These study blood samples are frozen and shipped to Astute Medical, Inc., San Diego, Calif. The study urine samples are frozen and shipped to Astute Medical, Inc.

### Example 3

### Acutely Ill Subject Sample Collection

The objective of this study is to collect samples from acutely ill patients. Approximately 1900 adults expected to be in the ICU for at least 48 hours will be enrolled. To be enrolled in the study, each patient must meet all of the following inclusion criteria and none of the following exclusion criteria:

### Inclusion Criteria

males and females 18 years of age or older;
Study population 1: approximately 300 patients that have at least one of:
shock (SBP<90 mmHg and/or need for vasopressor support to maintain MAP>60 mmHg and/or documented drop in SBP of at least 40 mmHg); and
sepsis;
Study population 2: approximately 300 patients that have at least one of:
IV antibiotics ordered in computerized physician order entry (CPOE) within 24 hours of enrollment;
contrast media exposure within 24 hours of enrollment;
increased Intra-Abdominal Pressure with acute decompensated heart failure; and severe trauma as the primary reason for ICU admission and likely to be hospitalized in the ICU for 48 hours after enrollment;
Study population 3: approximately 300 patients expected to be hospitalized through acute care setting (ICU or ED) with a known risk factor for acute renal injury (e.g. sepsis, hypotension/shock (Shock=systolic BP<90 mmHg and/or the need for vasopressor support to maintain a MAP>60 mmHg and/or a documented drop in SBP>40 mmHg), major trauma, hemorrhage, or major surgery); and/or expected to be hospitalized to the ICU for at least 24 hours after enrollment;
Study population 4: approximately 1000 patients that are 21 years of age or older, within 24 hours of being admitted into the ICU, expected to have an indwelling urinary catheter for at least 48 hours after enrollment, and have at least one of the following acute conditions within 24 hours prior to enrollment:
(i) respiratory SOFA score of >2 (PaO2/FiO2<300), (ii) cardiovascular SOFA score of >1 (MAP<70 mm Hg and/or any vasopressor required).

### Exclusion Criteria

known pregnancy;
institutionalized individuals;
previous renal transplantation;
known acutely worsening renal function prior to enrollment (e.g., any category of RIFLE criteria);
received dialysis (either acute or chronic) within 5 days prior to enrollment or in imminent need of dialysis at the time of enrollment;
known infection with human immunodeficiency virus (HIV) or a hepatitis virus;
meets any of the following:
(i) active bleeding with an anticipated need for >4 units PRBC in a day;
(ii) hemoglobin<7 g/dL;
(iii) any other condition that in the physician's opinion would contraindicate drawing serial blood samples for clinical study purposes;
meets only the SBP<90 mmHg inclusion criterion set forth above, and does not have shock in the attending physician's or principal investigator's opinion;

After obtaining informed consent, an EDTA anti-coagulated blood sample (10 mL) and a urine sample (25-50 mL) are collected from each patient. Blood and urine samples are then collected at 4 (±0.5) and 8 (±1) hours after contrast administration (if applicable); at 12 (±1), 24 (±2), 36 (±2), 48 (±2), 60 (±2), 72 (±2), and 84 (±2) hours after enrollment, and thereafter daily up to day 7 to day 14 while the subject is hospitalized. Blood is collected via direct venipuncture or via other available venous access, such as an existing femoral sheath, central venous line, peripheral intravenous line or hep-lock. These study blood samples are processed to plasma at the clinical site, frozen and shipped to Astute Medical, Inc., San Diego, Calif. The study urine samples are frozen and shipped to Astute Medical, Inc.

### Example 4

### Immunoassay Format

Analytes are measured using standard sandwich enzyme immunoassay techniques. A first antibody which binds the analyte is immobilized in wells of a 96 well polystyrene microplate. Analyte standards and test samples are pipetted into the appropriate wells and any analyte present is bound by the immobilized antibody. After washing away any unbound substances, a horseradish peroxidase-conjugated second antibody which binds the analyte is added to the wells, thereby forming sandwich complexes with the analyte (if present) and the first antibody. Following a wash to remove any unbound antibody-enzyme reagent, a substrate solution comprising tetramethylbenzidine and hydrogen peroxide is added to the wells. Color develops in proportion to the amount of analyte present in the sample. The color development is stopped and the intensity of the color is measured at 540 nm or 570 nm. An analyte concentration is assigned to the test sample by comparison to a standard curve determined from the analyte standards.

Units for the concentrations reported in the following data tables are as follows: Cancer antigen CA 15-3-U/mL, C-C Motif chemokine 18-ng/mL, C-C Motif chemokine 24-pg/mL, Cathepsin D-pg/mL, C-X-C Motif chemokine 13-pg/mL, C-C motif chemokine 8-pg/mL, Interleukin-2 receptor alpha chain-pg/mL, Insulin-like growth factor-binding protein 3-ng/mL, Interleukin-11-pg/mL, Matrix Metalloproteinase-8-pg/mL, Transforming growth factor alpha-pg/mL, IgG1-ng/mL, and IgG2-ng/mL. In the case of those kidney injury markers which are membrane proteins as described herein, the assays used in these examples detect soluble forms thereof.

Commercially-available reagents were sourced from the following vendors:

| Analyte | Assay Source | Catalog number |
|---|---|---|
| CA 15-3 | EMD Chemicals | Cat. # BPHCP001-6 |
| Cathepsin D | Millipore | Cat. #HNDG3-36K |
| C-C motif | Millipore | Cat. #HNDG2-36K |
| chemokine 18 | | |
| C-C motif | Millipore | Cat. # MPXHCYP2-62K |
| chemokine 24 | | |
| C-C motif | Millipore | Cat. # MPXHCYP2-62K |
| chemokine 8 | | |
| C-X-C motif | Millipore | Cat. # MPXHCYP2-62K |
| chemokine 13 | | |
| Immunoglobulin | Millipore | Cat. # HGAM-301 |
| G, subclass 1 | | |
| Immunoglobulin | Millipore | Cat. # HGAM-301 |
| G, subclass 2 | | |
| Insulin-like | Millipore | Cat. # HIGFBP-53K |
| growth factor- | | |
| binding protein 3 | | |
| Interleukin-11 | Millipore | Cat. # MPXHCYP3-63K |
| Interleukin-2 | Millipore | Cat. # MPXHCYTO-60K |
| receptor alpha | | |
| chain | | |
| Matrix | R&D Systems | Cat. # LMP000, Cat. # LMP908 |
| Metalloproteinase- | | |
| 8 | | |
| Transforming | Millipore | Cat. # MPXHCYTO-60K |
| growth factor | | |
| alpha | | |

### Example 5

### Apparently Healthy Donor and Chronic Disease Patient Samples

Human urine samples from donors with no known chronic or acute disease (“Apparently Healthy Donors”) were purchased from two vendors (Golden West Biologicals, Inc., 27625 Commerce Center Dr., Temecula, Calif. 92590 and Virginia Medical Research, Inc., 915 First Colonial Rd., Virginia Beach, Va. 23454). The urine samples were shipped and stored frozen at less than −20° C. The vendors supplied demographic information for the individual donors including gender, race (Black/White), smoking status and age.

Human urine samples from donors with various chronic diseases (“Chronic Disease Patients”) including congestive heart failure, coronary artery disease, chronic kidney disease, chronic obstructive pulmonary disease, diabetes mellitus and hypertension were purchased from Virginia Medical Research, Inc., 915 First Colonial Rd., Virginia Beach, Va. 23454. The urine samples were shipped and stored frozen at less than −20 degrees centigrade. The vendor provided a case report form for each individual donor with age, gender, race (Black/White), smoking status and alcohol use, height, weight, chronic disease(s) diagnosis, current medications and previous surgeries.

### Example 6

### Use of Kidney Injury Markers for Evaluating Renal Status in Patients

Patients from the intensive care unit (ICU) were enrolled in the following study. Each patient was classified by kidney status as non-injury (O), risk of injury (R), injury (I), and failure (F) according to the maximum stage reached within 7 days of enrollment as determined by the RIFLE criteria. EDTA anti-coagulated blood samples (10 mL) and a urine samples (25-30 mL) were collected from each patient at enrollment, 4 (±0.5) and 8 (±1) hours after contrast administration (if applicable); at 12 (±1), 24 (±2), and 48 (±2) hours after enrollment, and thereafter daily up to day 7 to day 14 while the subject is hospitalized. Markers were each measured by standard immunoassay methods using commercially available assay reagents in the urine samples and the plasma component of the blood samples collected.

Two cohorts were defined to represent a “diseased” and a “normal” population. While these terms are used for convenience, “diseased” and “normal” simply represent two cohorts for comparison (say RIFLE 0 vs RIFLE R, I and F; RIFLE 0 vs RIFLE R; RIFLE 0 and R vs RIFLE I and F; etc.). The time “prior max stage” represents the time at which a sample is collected, relative to the time a particular patient reaches the lowest disease stage as defined for that cohort, binned into three groups which are +/−12 hours. For example, “24 hr prior” which uses 0 vs R, I, F as the two cohorts would mean 24 hr (+/−12 hours) prior to reaching stage R (or I if no sample at R, or F if no sample at R or I).

A receiver operating characteristic (ROC) curve was generated for each biomarker measured and the area under each ROC curve (AUC) is determined. Patients in Cohort 2 were also separated according to the reason for adjudication to cohort 2 as being based on serum creatinine measurements (sCr), being based on urine output (UO), or being based on either serum creatinine measurements or urine output. Using the same example discussed above (0 vs R, I, F), for those patients adjudicated to stage R, I, or F on the basis of serum creatinine measurements alone, the stage 0 cohort may include patients adjudicated to stage R, I, or F on the basis of urine output; for those patients adjudicated to stage R, I, or F on the basis of urine output alone, the stage 0 cohort may include patients adjudicated to stage R, I, or F on the basis of serum creatinine measurements; and for those patients adjudicated to stage R, I, or F on the basis of serum creatinine measurements or urine output, the stage 0 cohort contains only patients in stage 0 for both serum creatinine measurements and urine output. Also, in the data for patients adjudicated on the basis of serum creatinine measurements or urine output, the adjudication method which yielded the most severe RIFLE stage is used.

The ability to distinguish cohort 1 from Cohort 2 was determined using ROC analysis. SE is the standard error of the AUC, n is the number of sample or individual patients (“pts,” as indicated). Standard errors are calculated as described in Hanley, J. A., and McNeil, B. J., The meaning and use of the area under a receiver operating characteristic (ROC) curve. Radiology (1982) 143: 29-36; p values are calculated with a two-tailed Z-test. An AUC<0.5 is indicative of a negative going marker for the comparison, and an AUC>0.5 is indicative of a positive going marker for the comparison.

Various threshold (or “cutoff”) concentrations were selected, and the associated sensitivity and specificity for distinguishing cohort 1 from cohort 2 are determined. OR is the odds ratio calculated for the particular cutoff concentration, and 95% CI is the confidence interval for the odds ratio.

TABLE 1
Comparison of marker levels in urine samples collected from Cohort 1 (patients that did not progress beyond RIFLE stage 0)
and in urine samples collected from subjects at 0, 24 hours, and 48 hours prior to reaching stage R, I or F in Cohort 2.
c-C motif chemokine 18

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 0.336 | 0.757 | 0.336 | 0.545 | 0.336 | 0.551 |
| Average | 2.29 | 3.44 | 2.29 | 2.98 | 2.29 | 2.60 |
| Stdev | 7.31 | 7.76 | 7.31 | 7.93 | 7.31 | 8.13 |
| p(t-test) | | 0.13 | | 0.35 | | 0.78 |
| Min | 3.13E−5 | 0.000952 | 3.13E−5 | 0.00194 | 3.13E−5 | 0.0160 |
| Max | 40.0 | 40.0 | 40.0 | 40.0 | 40.0 | 40.0 |
| n (Samp) | 463 | 119 | 463 | 128 | 463 | 47 |
| n (Patient) | 223 | 119 | 223 | 128 | 223 | 47 |
| sCr only | | | | | | |
| Median | 0.399 | 1.05 | 0.399 | 0.966 | 0.399 | 0.486 |
| Average | 2.26 | 5.09 | 2.26 | 2.77 | 2.26 | 3.91 |
| Stdev | 6.86 | 9.91 | 6.86 | 5.93 | 6.86 | 10.7 |
| p(t-test) | | 0.012 | | 0.62 | | 0.24 |
| Min | 3.13E−5 | 0.000952 | 3.13E−5 | 0.00497 | 3.13E−5 | 0.0172 |
| Max | 40.0 | 40.0 | 40.0 | 37.8 | 40.0 | 40.0 |
| n (Samp) | 1015 | 40 | 1015 | 46 | 1015 | 26 |
| n (Patient) | 374 | 40 | 374 | 46 | 374 | 26 |
| UO only | | | | | | |
| Median | 0.377 | 0.996 | 0.377 | 0.708 | 0.377 | 0.701 |
| Average | 2.54 | 4.28 | 2.54 | 4.16 | 2.54 | 3.83 |
| Stdev | 7.63 | 9.11 | 7.63 | 9.73 | 7.63 | 9.66 |
| p(t-test) | | 0.042 | | 0.054 | | 0.30 |
| Min | 3.13E−5 | 0.0153 | 3.13E−5 | 0.00194 | 3.13E−5 | 0.0160 |
| Max | 40.0 | 40.0 | 40.0 | 40.0 | 40.0 | 40.0 |
| n (Samp) | 436 | 107 | 436 | 117 | 436 | 44 |
| n (Patient) | 173 | 107 | 173 | 117 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.64 | 0.66 | 0.65 | 0.59 | 0.60 | 0.60 | 0.55 | 0.53 | 0.57 |
| SE | 0.030 | 0.048 | 0.031 | 0.029 | 0.045 | 0.030 | 0.045 | 0.058 | 0.047 |
| p | 1.1E−6 | 6.4E−4 | 1.9E−6 | 0.0024 | 0.024 | 0.0012 | 0.28 | 0.66 | 0.12 |
| nCohort 1 | 463 | 1015 | 436 | 463 | 1015 | 436 | 463 | 1015 | 436 |
| nCohort 2 | 119 | 40 | 107 | 128 | 46 | 117 | 47 | 26 | 44 |
| Cutoff 1 | 0.369 | 0.674 | 0.403 | 0.228 | 0.249 | 0.369 | 0.244 | 0.225 | 0.315 |
| Sens 1 | 71% | 70% | 70% | 70% | 72% | 70% | 70% | 73% | 70% |
| Spec 1 | 52% | 63% | 52% | 36% | 35% | 49% | 38% | 32% | 45% |
| Cutoff 2 | 0.223 | 0.315 | 0.258 | 0.142 | 0.150 | 0.177 | 0.134 | 0.129 | 0.162 |
| Sens 2 | 81% | 80% | 80% | 80% | 80% | 80% | 81% | 81% | 82% |
| Spec 2 | 35% | 44% | 36% | 26% | 24% | 27% | 25% | 20% | 25% |
| Cutoff 3 | 0.111 | 0.138 | 0.138 | 0.0629 | 0.0568 | 0.0751 | 0.0403 | 0.0403 | 0.124 |
| Sens 3 | 91% | 90% | 91% | 91% | 91% | 91% | 91% | 92% | 91% |
| Spec 3 | 21% | 21% | 21% | 12% | 8% | 13% | 9% | 7% | 20% |
| Cutoff 4 | 0.751 | 0.893 | 0.906 | 0.751 | 0.893 | 0.906 | 0.751 | 0.893 | 0.906 |
| Sens 4 | 50% | 62% | 50% | 45% | 52% | 46% | 40% | 35% | 36% |
| Spec 4 | 70% | 70% | 70% | 70% | 70% | 70% | 70% | 70% | 70% |
| Cutoff 5 | 1.12 | 1.51 | 1.63 | 1.12 | 1.51 | 1.63 | 1.12 | 1.51 | 1.63 |
| Sens 5 | 40% | 45% | 37% | 37% | 39% | 32% | 21% | 27% | 20% |
| Spec 5 | 80% | 80% | 80% | 80% | 80% | 80% | 80% | 80% | 80% |
| Cutoff 6 | 3.17 | 3.53 | 3.59 | 3.17 | 3.53 | 3.59 | 3.17 | 3.53 | 3.59 |
| Sens 6 | 24% | 22% | 26% | 16% | 20% | 16% | 9% | 12% | 11% |
| Spec 6 | 90% | 90% | 90% | 90% | 90% | 90% | 90% | 90% | 90% |
| OR Quart 2 | 1.3 | 1.2 | 1.6 | 0.73 | 0.39 | 0.75 | 0.78 | 0.83 | 0.65 |
| p Value | 0.40 | 0.77 | 0.22 | 0.32 | 0.12 | 0.40 | 0.61 | 0.76 | 0.43 |
| 95% CI of | 0.68 | 0.36 | 0.77 | 0.39 | 0.12 | 0.38 | 0.30 | 0.25 | 0.22 |
| OR Quart2 | 2.6 | 4.0 | 3.3 | 1.4 | 1.3 | 1.5 | 2.0 | 2.8 | 1.9 |
| OR Quart 3 | 2.0 | 2.0 | 2.2 | 1.3 | 1.2 | 1.6 | 1.7 | 1.3 | 2.0 |
| p Value | 0.029 | 0.20 | 0.022 | 0.32 | 0.66 | 0.14 | 0.22 | 0.59 | 0.10 |
| 95% CI of | 1.1 | 0.68 | 1.1 | 0.75 | 0.51 | 0.87 | 0.73 | 0.46 | 0.87 |
| OR Quart3 | 3.9 | 6.0 | 4.5 | 2.4 | 2.8 | 2.8 | 3.9 | 3.9 | 4.8 |
| OR Quart 4 | 3.8 | 4.0 | 4.1 | 2.3 | 2.1 | 2.2 | 1.3 | 1.2 | 1.4 |
| p Value | 1.9E−5 | 0.0066 | 2.5E−5 | 0.0027 | 0.067 | 0.0059 | 0.53 | 0.78 | 0.49 |
| 95% CI of | 2.1 | 1.5 | 2.1 | 1.3 | 0.95 | 1.3 | 0.56 | 0.39 | 0.55 |
| OR Quart4 | 7.0 | 11 | 8.0 | 4.0 | 4.5 | 4.0 | 3.1 | 3.5 | 3.4 |

C-C motif chemokine 24

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 8.31 | 20.6 | 8.31 | 15.9 | 8.31 | 9.14 |
| Average | 19.2 | 46.8 | 19.2 | 57.8 | 19.2 | 18.4 |
| Stdev | 49.6 | 108 | 49.6 | 237 | 49.6 | 34.6 |
| p(t-test) | | 4.9E−5 | | 0.0012 | | 0.91 |
| Min | 0.0120 | 0.0160 | 0.0120 | 0.0120 | 0.0120 | 0.0193 |
| Max | 730 | 1010 | 730 | 2380 | 730 | 216 |
| n (Samp) | 463 | 120 | 463 | 130 | 463 | 47 |
| n (Patient) | 223 | 120 | 223 | 130 | 223 | 47 |
| sCr only | | | | | | |
| Median | 11.9 | 28.7 | 11.9 | 20.3 | 11.9 | 13.7 |
| Average | 31.8 | 46.7 | 31.8 | 39.1 | 31.8 | 31.2 |
| Stdev | 113 | 62.4 | 113 | 63.5 | 113 | 54.6 |
| p(t-test) | | 0.41 | | 0.66 | | 0.98 |
| Min | 0.0120 | 0.0160 | 0.0120 | 0.0196 | 0.0120 | 0.0215 |
| Max | 2380 | 290 | 2380 | 347 | 2380 | 216 |
| n (Samp) | 1019 | 40 | 1019 | 46 | 1019 | 26 |
| n (Patient) | 375 | 40 | 375 | 46 | 375 | 26 |
| UO only | | | | | | |
| Median | 8.53 | 25.8 | 8.53 | 18.2 | 8.53 | 13.1 |
| Average | 21.8 | 85.0 | 21.8 | 86.7 | 21.8 | 49.4 |
| Stdev | 53.7 | 298 | 53.7 | 309 | 53.7 | 182 |
| p(t-test) | | 3.6E−5 | | 3.6E−5 | | 0.020 |
| Min | 0.0120 | 0.0160 | 0.0120 | 0.0120 | 0.0120 | 0.0160 |
| Max | 730 | 2790 | 730 | 2380 | 730 | 1170 |
| n (Samp) | 435 | 108 | 435 | 119 | 435 | 44 |
| n (Patient) | 173 | 108 | 173 | 119 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.66 | 0.62 | 0.69 | 0.63 | 0.59 | 0.65 | 0.52 | 0.50 | 0.56 |
| SE | 0.029 | 0.048 | 0.030 | 0.029 | 0.045 | 0.030 | 0.045 | 0.058 | 0.047 |
| p | 2.7E−8 | 0.0095 | 1.5E−10 | 7.5E−6 | 0.035 | 1.1E−6 | 0.66 | 0.94 | 0.22 |
| nCohort 1 | 463 | 1019 | 435 | 463 | 1019 | 435 | 463 | 1019 | 435 |
| nCohort 2 | 120 | 40 | 108 | 130 | 46 | 119 | 47 | 26 | 44 |
| Cutoff 1 | 8.53 | 10.4 | 11.5 | 7.45 | 9.88 | 8.20 | 3.27 | 1.48 | 5.35 |
| Sens 1 | 70% | 70% | 70% | 70% | 72% | 71% | 70% | 73% | 70% |
| Spec 1 | 51% | 47% | 59% | 48% | 46% | 49% | 35% | 22% | 42% |
| Cutoff 2 | 4.36 | 0.405 | 6.04 | 3.71 | 3.71 | 4.18 | 0.405 | 0.0635 | 1.03 |
| Sens 2 | 80% | 80% | 81% | 80% | 80% | 81% | 81% | 81% | 82% |
| Spec 2 | 38% | 20% | 43% | 36% | 28% | 38% | 28% | 18% | 28% |
| Cutoff 3 | 0.0469 | 0.0344 | 0.0562 | 0.0552 | 0.0469 | 0.0562 | 0.0235 | 0.0277 | 0.0263 |
| Sens 3 | 91% | 90% | 91% | 90% | 91% | 91% | 94% | 92% | 91% |
| Spec 3 | 19% | 11% | 23% | 22% | 13% | 23% | 8% | 7% | 7% |
| Cutoff 4 | 16.0 | 23.8 | 16.7 | 16.0 | 23.8 | 16.7 | 16.0 | 23.8 | 16.7 |
| Sens 4 | 57% | 57% | 61% | 50% | 46% | 50% | 36% | 38% | 41% |
| Spec 4 | 70% | 70% | 70% | 70% | 70% | 70% | 70% | 70% | 70% |
| Cutoff 5 | 25.6 | 36.4 | 25.7 | 25.6 | 36.4 | 25.7 | 25.6 | 36.4 | 25.7 |
| Sens 5 | 46% | 40% | 50% | 35% | 28% | 39% | 15% | 23% | 20% |
| Spec 5 | 80% | 80% | 80% | 80% | 80% | 80% | 80% | 80% | 80% |
| Cutoff 6 | 40.7 | 62.3 | 48.2 | 40.7 | 62.3 | 48.2 | 40.7 | 62.3 | 48.2 |
| Sens 6 | 30% | 20% | 28% | 22% | 15% | 22% | 9% | 12% | 9% |
| Spec 6 | 90% | 90% | 90% | 90% | 90% | 90% | 90% | 90% | 90% |
| OR Quart 2 | 1.2 | 0.54 | 1.8 | 2.0 | 0.87 | 2.2 | 2.0 | 0.55 | 1.6 |
| p Value | 0.62 | 0.28 | 0.14 | 0.041 | 0.79 | 0.023 | 0.14 | 0.29 | 0.34 |
| 95% CI of | 0.60 | 0.18 | 0.83 | 1.0 | 0.31 | 1.1 | 0.81 | 0.18 | 0.60 |
| OR Quart2 | 2.3 | 1.6 | 3.8 | 3.7 | 2.4 | 4.5 | 4.8 | 1.7 | 4.3 |
| OR Quart 3 | 1.5 | 0.77 | 2.2 | 2.2 | 1.8 | 2.6 | 1.4 | 0.33 | 2.1 |
| p Value | 0.20 | 0.61 | 0.037 | 0.015 | 0.20 | 0.0068 | 0.48 | 0.096 | 0.12 |
| 95% CI of | 0.80 | 0.28 | 1.0 | 1.2 | 0.74 | 1.3 | 0.55 | 0.087 | 0.82 |
| OR Quart3 | 2.9 | 2.1 | 4.6 | 4.2 | 4.3 | 5.1 | 3.6 | 1.2 | 5.4 |
| OR Quart 4 | 4.3 | 2.2 | 6.3 | 3.9 | 2.2 | 4.4 | 1.7 | 1.0 | 1.8 |
| p Value | 1.9E−6 | 0.059 | 1.3E−7 | 1.2E−5 | 0.073 | 1.0E−5 | 0.27 | 0.99 | 0.24 |
| 95% CI of | 2.3 | 0.97 | 3.2 | 2.1 | 0.93 | 2.3 | 0.67 | 0.39 | 0.67 |
| OR Quart4 | 7.7 | 4.9 | 13 | 7.2 | 5.2 | 8.4 | 4.2 | 2.6 | 4.7 |

C-C motif chemokine 8

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 1.21 | 1.72 | 1.21 | 2.97 | 1.21 | 2.77 |
| Average | 10.1 | 9.75 | 10.1 | 13.6 | 10.1 | 10.8 |
| Stdev | 24.9 | 21.6 | 24.9 | 33.2 | 24.9 | 34.3 |
| p(t-test) | | 0.88 | | 0.19 | | 0.86 |
| Min | 0.0250 | 0.0635 | 0.0250 | 0.0250 | 0.0250 | 0.0250 |
| Max | 250 | 158 | 250 | 294 | 250 | 213 |
| n (Samp) | 463 | 120 | 463 | 130 | 463 | 47 |
| n (Patient) | 223 | 120 | 223 | 130 | 223 | 47 |
| sCr only | | | | | | |
| Median | 1.36 | 2.23 | 1.36 | 6.03 | 1.36 | 2.14 |
| Average | 10.6 | 16.4 | 10.6 | 20.7 | 10.6 | 18.6 |
| Stdev | 32.3 | 36.8 | 32.3 | 46.5 | 32.3 | 45.7 |
| p(t-test) | | 0.27 | | 0.042 | | 0.22 |
| Min | 0.0250 | 0.0635 | 0.0250 | 0.0250 | 0.0250 | 0.0250 |
| Max | 492 | 185 | 492 | 279 | 492 | 213 |
| n (Samp) | 1019 | 40 | 1019 | 46 | 1019 | 26 |
| n (Patient) | 375 | 40 | 375 | 46 | 375 | 26 |
| UO only | | | | | | |
| Median | 1.21 | 2.59 | 1.21 | 2.77 | 1.21 | 2.16 |
| Average | 10.6 | 12.3 | 10.6 | 16.1 | 10.6 | 12.5 |
| Stdev | 26.0 | 27.1 | 26.0 | 36.2 | 26.0 | 37.4 |
| p(t-test) | | 0.54 | | 0.066 | | 0.67 |
| Min | 0.0250 | 0.0701 | 0.0250 | 0.0250 | 0.0250 | 0.0472 |
| Max | 250 | 176 | 250 | 294 | 250 | 214 |
| n (Samp) | 435 | 108 | 435 | 119 | 435 | 44 |
| n (Patient) | 173 | 108 | 173 | 119 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.57 | 0.58 | 0.59 | 0.60 | 0.62 | 0.60 | 0.56 | 0.55 | 0.53 |
| SE | 0.030 | 0.048 | 0.032 | 0.029 | 0.045 | 0.030 | 0.045 | 0.059 | 0.046 |
| p | 0.017 | 0.092 | 0.0041 | 9.0E−4 | 0.0090 | 8.1E−4 | 0.21 | 0.42 | 0.47 |
| nCohort 1 | 463 | 1019 | 435 | 463 | 1019 | 435 | 463 | 1019 | 435 |
| nCohort 2 | 120 | 40 | 108 | 130 | 46 | 119 | 47 | 26 | 44 |
| Cutoff 1 | 0.453 | 0.802 | 0.802 | 0.491 | 0.802 | 0.567 | 0.802 | 0.802 | 0.491 |
| Sens 1 | 72% | 72% | 73% | 73% | 74% | 71% | 74% | 73% | 70% |
| Spec 1 | 43% | 44% | 46% | 43% | 44% | 45% | 47% | 44% | 42% |
| Cutoff 2 | 0.154 | 0.240 | 0.239 | 0.239 | 0.239 | 0.240 | 0.239 | 0.136 | 0.154 |
| Sens 2 | 82% | 85% | 81% | 80% | 80% | 81% | 81% | 81% | 82% |
| Spec 2 | 33% | 35% | 33% | 36% | 32% | 36% | 36% | 25% | 29% |
| Cutoff 3 | 0.0945 | 0.127 | 0.127 | 0.0775 | 0.0775 | 0.127 | 0.0569 | 0.0705 | 0.0701 |
| Sens 3 | 90% | 90% | 91% | 92% | 93% | 91% | 94% | 92% | 91% |
| Spec 3 | 22% | 22% | 23% | 18% | 14% | 23% | 10% | 12% | 12% |
| Cutoff 4 | 5.70 | 6.40 | 5.77 | 5.70 | 6.40 | 5.77 | 5.70 | 6.40 | 5.77 |
| Sens 4 | 33% | 38% | 39% | 41% | 50% | 41% | 23% | 35% | 20% |
| Spec 4 | 70% | 70% | 70% | 70% | 70% | 70% | 70% | 70% | 70% |
| Cutoff 5 | 11.1 | 11.3 | 11.7 | 11.1 | 11.3 | 11.7 | 11.1 | 11.3 | 11.7 |
| Sens 5 | 19% | 22% | 24% | 25% | 35% | 24% | 13% | 19% | 14% |
| Spec 5 | 80% | 80% | 80% | 80% | 80% | 80% | 80% | 80% | 80% |
| Cutoff 6 | 28.3 | 24.5 | 25.3 | 28.3 | 24.5 | 25.3 | 28.3 | 24.5 | 25.3 |
| Sens 6 | 9% | 12% | 15% | 12% | 20% | 16% | 4% | 12% | 7% |
| Spec 6 | 90% | 90% | 90% | 90% | 90% | 90% | 90% | 90% | 90% |
| OR Quart 2 | 2.4 | 2.5 | 1.8 | 1.8 | 0.87 | 1.8 | 1.1 | 1.0 | 1.3 |
| p Value | 0.0066 | 0.096 | 0.074 | 0.061 | 0.79 | 0.075 | 0.80 | 1.0 | 0.62 |
| 95% CI of | 1.3 | 0.85 | 0.94 | 0.97 | 0.31 | 0.94 | 0.40 | 0.32 | 0.47 |
| OR Quart2 | 4.7 | 7.1 | 3.6 | 3.5 | 2.4 | 3.6 | 3.3 | 3.1 | 3.6 |
| OR Quart 3 | 3.4 | 2.5 | 2.7 | 3.0 | 1.5 | 3.1 | 4.0 | 1.3 | 3.2 |
| p Value | 1.6E−4 | 0.096 | 0.0028 | 4.7E−4 | 0.37 | 4.9E−4 | 0.0021 | 0.59 | 0.011 |
| 95% CI of | 1.8 | 0.85 | 1.4 | 1.6 | 0.61 | 1.6 | 1.7 | 0.46 | 1.3 |
| OR Quart3 | 6.3 | 7.1 | 5.1 | 5.4 | 3.8 | 5.9 | 9.7 | 3.9 | 7.9 |
| OR Quart 4 | 1.8 | 2.2 | 2.0 | 2.6 | 2.5 | 2.7 | 1.1 | 1.00 | 1.1 |
| p Value | 0.076 | 0.14 | 0.038 | 0.0027 | 0.036 | 0.0029 | 0.80 | 0.99 | 0.80 |
| 95% CI of | 0.94 | 0.77 | 1.0 | 1.4 | 1.1 | 1.4 | 0.40 | 0.32 | 0.40 |
| OR Quart4 | 3.6 | 6.5 | 3.9 | 4.7 | 5.7 | 5.1 | 3.3 | 3.1 | 3.3 |

Cathepsin D

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 65300 | 94300 | 65300 | 87400 | 65300 | 69000 |
| Average | 69400 | 98700 | 69400 | 88000 | 69400 | 77300 |
| Stdev | 35600 | 44500 | 35600 | 43900 | 35600 | 42200 |
| p(t-test) | | 1.3E−13 | | 9.3E−7 | | 0.16 |
| Min | 656 | 2920 | 656 | 3320 | 656 | 4810 |
| Max | 200000 | 200000 | 200000 | 200000 | 200000 | 200000 |
| n (Samp) | 463 | 119 | 463 | 128 | 463 | 47 |
| n (Patient) | 223 | 119 | 223 | 128 | 223 | 47 |
| sCr only | | | | | | |
| Median | 74500 | 106000 | 74500 | 85500 | 74500 | 75700 |
| Average | 79700 | 104000 | 79700 | 94500 | 79700 | 79600 |
| Stdev | 40600 | 50300 | 40600 | 50700 | 40600 | 47400 |
| p(t-test) | | 2.7E−4 | | 0.017 | | 0.99 |
| Min | 656 | 2920 | 656 | 4880 | 656 | 5800 |
| Max | 200000 | 200000 | 200000 | 200000 | 200000 | 200000 |
| n (Samp) | 1015 | 40 | 1015 | 46 | 1015 | 26 |
| n (Patient) | 374 | 40 | 374 | 46 | 374 | 26 |
| UO only | | | | | | |
| Median | 70200 | 99000 | 70200 | 88700 | 70200 | 80600 |
| Average | 75500 | 103000 | 75500 | 93900 | 75500 | 83900 |
| Stdev | 38400 | 43300 | 38400 | 42600 | 38400 | 41000 |
| p(t-test) | | 1.6E−10 | | 8.5E−6 | | 0.17 |
| Min | 2520 | 12600 | 2520 | 3320 | 2520 | 4810 |
| Max | 200000 | 200000 | 200000 | 200000 | 200000 | 200000 |
| n (Samp) | 436 | 107 | 436 | 117 | 436 | 44 |
| n (Patient) | 173 | 107 | 173 | 117 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.70 | 0.65 | 0.69 | 0.63 | 0.59 | 0.63 | 0.55 | 0.48 | 0.57 |
| SE | 0.029 | 0.048 | 0.031 | 0.029 | 0.045 | 0.030 | 0.045 | 0.058 | 0.047 |
| p | 1.1E−11 | 0.0015 | 6.3E−10 | 6.6E−6 | 0.037 | 8.8E−6 | 0.23 | 0.78 | 0.14 |
| nCohort 1 | 463 | 1015 | 436 | 463 | 1015 | 436 | 463 | 1015 | 436 |
| nCohort 2 | 119 | 40 | 107 | 128 | 46 | 1.17 | 47 | 26 | 44 |
| Cutoff 1 | 72900 | 81200 | 74300 | 65000 | 70400 | 70900 | 56000 | 46800 | 61900 |
| Sens 1 | 71% | 70% | 70% | 70% | 72% | 70% | 70% | 73% | 70% |
| Spec 1 | 58% | 57% | 55% | 50% | 46% | 52% | 38% | 21% | 41% |
| Cutoff 2 | 62500 | 63200 | 69000 | 52000 | 58400 | 58800 | 45500 | 42000 | 54100 |
| Sens 2 | 81% | 80% | 80% | 80% | 80% | 80% | 81% | 81% | 82% |
| Spec 2 | 46% | 37% | 48% | 32% | 32% | 38% | 25% | 18% | 31% |
| Cutoff 3 | 43600 | 43100 | 54100 | 26100 | 19700 | 41500 | 22000 | 22000 | 39600 |
| Sens 3 | 91% | 90% | 91% | 91% | 91% | 91% | 91% | 92% | 91% |
| Spec 3 | 24% | 18% | 31% | 11% | 5% | 18% | 8% | 6% | 16% |
| Cutoff 4 | 82600 | 98000 | 90300 | 82600 | 98000 | 90300 | 82600 | 98000 | 90300 |
| Sens 4 | 60% | 52% | 55% | 55% | 41% | 48% | 40% | 31% | 41% |
| Spec 4 | 70% | 70% | 70% | 70% | 70% | 70% | 70% | 70% | 70% |
| Cutoff 5 | 99400 | 112000 | 108000 | 99400 | 112000 | 108000 | 99400 | 112000 | 108000 |
| Sens 5 | 48% | 40% | 43% | 36% | 37% | 37% | 23% | 15% | 20% |
| Spec 5 | 80% | 80% | 80% | 80% | 80% | 80% | 80% | 80% | 80% |
| Cutoff 6 | 121000 | 135000 | 126000 | 121000 | 135000 | 126000 | 121000 | 135000 | 126000 |
| Sens 6 | 29% | 25% | 27% | 21% | 20% | 22% | 13% | 8% | 18% |
| Spec 6 | 90% | 90% | 90% | 90% | 90% | 90% | 90% | 90% | 90% |
| OR Quart 2 | 1.1 | 0.56 | 2.3 | 1.0 | 1.0 | 1.2 | 0.99 | 0.62 | 2.0 |
| p Value | 0.72 | 0.36 | 0.042 | 0.89 | 1.0 | 0.60 | 0.99 | 0.41 | 0.17 |
| 95% CI of | 0.55 | 0.16 | 1.0 | 0.55 | 0.37 | 0.61 | 0.40 | 0.20 | 0.75 |
| OR Quart2 | 2.4 | 1.9 | 5.1 | 2.0 | 2.7 | 2.4 | 2.5 | 1.9 | 5.1 |
| OR Quart 3 | 2.4 | 1.3 | 3.4 | 1.9 | 1.7 | 2.0 | 1.2 | 0.62 | 1.6 |
| p Value | 0.012 | 0.62 | 0.0017 | 0.042 | 0.27 | 0.031 | 0.66 | 0.41 | 0.33 |
| 95% CI of | 1.2 | 0.47 | 1.6 | 1.0 | 0.68 | 1.1 | 0.51 | 0.20 | 0.61 |
| OR Quart3 | 4.6 | 3.5 | 7.3 | 3.4 | 4.1 | 3.8 | 2.9 | 1.9 | 4.4 |
| OR Quart 4 | 5.4 | 3.0 | 6.6 | 3.1 | 2.2 | 3.3 | 1.6 | 1.0 | 2.0 |
| p Value | 1.6E−7 | 0.014 | 4.8E−7 | 1.3E−4 | 0.073 | 1.2E−4 | 0.30 | 0.99 | 0.17 |
| 95% CI of | 2.9 | 1.2 | 3.2 | 1.7 | 0.93 | 1.8 | 0.67 | 0.37 | 0.75 |
| OR Quart4 | 10 | 7.2 | 14 | 5.4 | 5.2 | 6.1 | 3.6 | 2.7 | 5.1 |

C-X-C motif chemokine 13

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 0.0163 | 1.10 | 0.0163 | 0.590 | 0.0163 | 0.448 |
| Average | 8.44 | 7.15 | 8.44 | 7.71 | 8.44 | 6.34 |
| Stdev | 53.2 | 23.6 | 53.2 | 21.5 | 53.2 | 16.8 |
| p(t-test) | | 0.80 | | 0.88 | | 0.79 |
| Min | 0.00269 | 0.00269 | 0.00269 | 0.00269 | 0.00269 | 0.00269 |
| Max | 832 | 210 | 832 | 131 | 832 | 75.7 |
| n (Samp) | 463 | 120 | 463 | 130 | 463 | 47 |
| n (Patient) | 223 | 120 | 223 | 130 | 223 | 47 |
| sCr only | | | | | | |
| Median | 0.0222 | 1.41 | 0.0222 | 0.880 | 0.0222 | 0.520 |
| Average | 7.21 | 16.4 | 7.21 | 8.42 | 7.21 | 6.73 |
| Stdev | 40.5 | 51.0 | 40.5 | 20.0 | 40.5 | 17.0 |
| p(t-test) | | 0.16 | | 0.84 | | 0.95 |
| Min | 0.00269 | 0.00471 | 0.00269 | 0.00269 | 0.00269 | 0.00269 |
| Max | 832 | 252 | 832 | 79.1 | 832 | 77.1 |
| n (Samp) | 1019 | 40 | 1019 | 46 | 1019 | 26 |
| n (Patient) | 375 | 40 | 375 | 46 | 375 | 26 |
| UO only | | | | | | |
| Median | 0.0163 | 1.68 | 0.0163 | 0.772 | 0.0163 | 0.644 |
| Average | 8.43 | 27.2 | 8.43 | 26.4 | 8.43 | 16.1 |
| Stdev | 54.5 | 187 | 54.5 | 171 | 54.5 | 67.5 |
| p(t-test) | | 0.072 | | 0.062 | | 0.39 |
| Min | 0.00269 | 0.00269 | 0.00269 | 0.00269 | 0.00269 | 0.00474 |
| Max | 832 | 1930 | 832 | 1850 | 832 | 440 |
| n (Samp) | 435 | 108 | 435 | 119 | 435 | 44 |
| n (Patient) | 173 | 108 | 173 | 119 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.64 | 0.63 | 0.68 | 0.58 | 0.58 | 0.61 | 0.57 | 0.54 | 0.61 |
| SE | 0.030 | 0.048 | 0.031 | 0.029 | 0.045 | 0.030 | 0.045 | 0.059 | 0.047 |
| p | 2.9E−6 | 0.0070 | 6.2E−9 | 0.0096 | 0.075 | 2.8E−4 | 0.14 | 0.50 | 0.020 |
| nCohort 1 | 463 | 1019 | 435 | 463 | 1019 | 435 | 463 | 1019 | 435 |
| nCohort 2 | 120 | 40 | 108 | 130 | 46 | 119 | 47 | 26 | 44 |
| Cutoff 1 | 0.0222 | 0.0193 | 0.392 | 0.0126 | 0.0126 | 0.0155 | 0.0151 | 0.0130 | 0.0174 |
| Sens 1 | 70% | 72% | 70% | 72% | 72% | 72% | 70% | 73% | 70% |
| Spec 1 | 55% | 47% | 62% | 37% | 32% | 48% | 44% | 34% | 50% |
| Cutoff 2 | 0.0126 | 0.0126 | 0.0193 | 0.00821 | 0.00821 | 0.00930 | 0.00821 | 0.0104 | 0.00930 |
| Sens 2 | 82% | 80% | 81% | 83% | 87% | 83% | 81% | 81% | 82% |
| Spec 2 | 37% | 32% | 51% | 26% | 21% | 27% | 26% | 27% | 27% |
| Cutoff 3 | 0.00821 | 0.0104 | 0.00821 | 0.00564 | 0.00789 | 0.00564 | 0.00471 | 0.00471 | 0.00789 |
| Sens 3 | 90% | 90% | 92% | 91% | 91% | 92% | 96% | 92% | 91% |
| Spec 3 | 26% | 27% | 22% | 17% | 18% | 14% | 10% | 8% | 20% |
| Cutoff 4 | 1.14 | 1.32 | 1.05 | 1.14 | 1.32 | 1.05 | 1.14 | 1.32 | 1.05 |
| Sens 4 | 49% | 50% | 58% | 36% | 43% | 44% | 34% | 38% | 41% |
| Spec 4 | 70% | 70% | 70% | 70% | 70% | 70% | 70% | 70% | 70% |
| Cutoff 5 | 3.19 | 3.33 | 2.67 | 3.19 | 3.33 | 2.67 | 3.19 | 3.33 | 2.67 |
| Sens 5 | 28% | 35% | 38% | 22% | 28% | 29% | 19% | 19% | 27% |
| Spec 5 | 80% | 80% | 80% | 80% | 80% | 80% | 80% | 80% | 80% |
| Cutoff 6 | 10.2 | 10.2 | 8.59 | 10.2 | 10.2 | 8.59 | 10.2 | 10.2 | 8.59 |
| Sens 6 | 13% | 20% | 18% | 14% | 15% | 22% | 13% | 15% | 16% |
| Spec 6 | 90% | 90% | 90% | 90% | 90% | 90% | 90% | 90% | 90% |
| OR Quart 2 | 1.7 | 2.3 | 1.1 | 1.3 | 0.63 | 1.0 | 1.3 | 1.2 | 0.99 |
| p Value | 0.16 | 0.17 | 0.86 | 0.44 | 0.34 | 0.89 | 0.64 | 0.76 | 0.99 |
| 95% CI of | 0.81 | 0.69 | 0.49 | 0.69 | 0.24 | 0.54 | 0.48 | 0.36 | 0.34 |
| OR Quart2 | 3.4 | 7.5 | 2.4 | 2.3 | 1.6 | 2.0 | 3.3 | 4.0 | 2.9 |
| OR Quart 3 | 3.8 | 2.5 | 3.5 | 2.2 | 1.2 | 2.3 | 2.1 | 1.8 | 2.1 |
| p Value | 7.4E−5 | 0.12 | 3.3E−4 | 0.0084 | 0.68 | 0.0060 | 0.092 | 0.29 | 0.12 |
| 95% CI of | 2.0 | 0.79 | 1.8 | 1.2 | 0.52 | 1.3 | 0.88 | 0.60 | 0.82 |
| OR Quart3 | 7.3 | 8.2 | 7.0 | 3.8 | 2.7 | 4.2 | 5.2 | 5.5 | 5.4 |
| OR Quart 4 | 3.8 | 4.5 | 4.5 | 1.8 | 1.4 | 2.3 | 1.7 | 1.2 | 2.5 |
| p Value | 7.4E−5 | 0.0079 | 1.3E−5 | 0.048 | 0.43 | 0.0066 | 0.27 | 0.77 | 0.057 |
| 95% CI of | 2.0 | 1.5 | 2.3 | 1.0 | 0.62 | 1.3 | 0.67 | 0.36 | 0.97 |
| OR Quart4 | 7.3 | 13 | 8.8 | 3.2 | 3.1 | 4.2 | 4.2 | 4.0 | 6.2 |

Insulin-like growth factor-binding protein 3

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 298 | 508 | 298 | 491 | 298 | 620 |
| Average | 559 | 748 | 559 | 1030 | 559 | 733 |
| Stdev | 935 | 758 | 935 | 1970 | 935 | 684 |
| p(t-test) | | 0.027 | | 6.9E−5 | | 0.21 |
| Min | 3.84 | 5.67 | 3.84 | 8.21 | 3.84 | 3.83 |
| Max | 12500 | 4890 | 12500 | 12500 | 12500 | 3220 |
| n (Samp) | 499 | 142 | 499 | 141 | 499 | 48 |
| n (Patient) | 235 | 142 | 235 | 141 | 235 | 48 |
| sCr only | | | | | | |
| Median | 388 | 432 | 388 | 376 | 388 | 423 |
| Average | 665 | 827 | 665 | 917 | 665 | 635 |
| Stdev | 1060 | 1290 | 1060 | 1400 | 1060 | 583 |
| p(t-test) | | 0.32 | | 0.10 | | 0.88 |
| Min | 0.0478 | 5.67 | 0.0478 | 14.5 | 0.0478 | 3.83 |
| Max | 12500 | 7720 | 12500 | 8160 | 12500 | 1890 |
| n (Samp) | 1097 | 46 | 1097 | 51 | 1097 | 28 |
| n (Patient) | 397 | 46 | 397 | 51 | 397 | 28 |
| UO only | | | | | | |
| Median | 311 | 599 | 311 | 590 | 311 | 699 |
| Average | 617 | 1010 | 617 | 1130 | 617 | 771 |
| Stdev | 1150 | 1400 | 1150 | 2020 | 1150 | 659 |
| p(t-test) | | 0.0012 | | 1.9E−4 | | 0.37 |
| Min | 3.84 | 21.2 | 3.84 | 8.21 | 3.84 | 10.2 |
| Max | 12500 | 12500 | 12500 | 12500 | 12500 | 3220 |
| n (Samp) | 487 | 129 | 487 | 131 | 487 | 47 |
| n (Patient) | 190 | 129 | 190 | 131 | 190 | 47 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.63 | 0.52 | 0.67 | 0.60 | 0.53 | 0.63 | 0.62 | 0.52 | 0.64 |
| SE | 0.028 | 0.044 | 0.028 | 0.028 | 0.042 | 0.029 | 0.045 | 0.056 | 0.045 |
| p | 5.3E−6 | 0.58 | 1.7E−9 | 3.0E−4 | 0.43 | 3.4E−6 | 0.0095 | 0.68 | 0.0020 |
| nCohort 1 | 499 | 1097 | 487 | 499 | 1097 | 487 | 499 | 1097 | 487 |
| nCohort 2 | 142 | 46 | 129 | 141 | 51 | 131 | 48 | 28 | 47 |
| Cutoff 1 | 275 | 205 | 349 | 225 | 202 | 314 | 219 | 174 | 369 |
| Sens 1 | 70% | 72% | 71% | 70% | 71% | 70% | 71% | 71% | 70% |
| Spec 1 | 48% | 32% | 54% | 42% | 32% | 51% | 41% | 28% | 56% |
| Cutoff 2 | 191 | 136 | 257 | 116 | 163 | 176 | 145 | 110 | 191 |
| Sens 2 | 80% | 80% | 81% | 80% | 80% | 80% | 81% | 82% | 81% |
| Spec 2 | 39% | 23% | 45% | 28% | 26% | 34% | 32% | 20% | 36% |
| Cutoff 3 | 105 | 27.5 | 141 | 46.2 | 43.4 | 80.9 | 51.9 | 43.4 | 77.9 |
| Sens 3 | 90% | 91% | 91% | 90% | 90% | 90% | 92% | 93% | 91% |
| Spec 3 | 27% | 5% | 30% | 11% | 7% | 19% | 14% | 7% | 19% |
| Cutoff 4 | 585 | 710 | 587 | 585 | 710 | 587 | 585 | 710 | 587 |
| Sens 4 | 43% | 35% | 51% | 44% | 41% | 50% | 52% | 36% | 55% |
| Spec 4 | 70% | 70% | 70% | 70% | 70% | 70% | 70% | 70% | 70% |
| Cutoff 5 | 829 | 997 | 859 | 829 | 997 | 859 | 829 | 997 | 859 |
| Sens 5 | 34% | 26% | 40% | 36% | 27% | 40% | 35% | 29% | 32% |
| Spec 5 | 80% | 80% | 80% | 80% | 80% | 80% | 80% | 80% | 80% |
| Cutoff 6 | 1260 | 1420 | 1320 | 1260 | 1420 | 1320 | 1260 | 1420 | 1320 |
| Sens 6 | 20% | 17% | 22% | 21% | 18% | 21% | 19% | 14% | 15% |
| Spec 6 | 90% | 90% | 90% | 90% | 90% | 90% | 90% | 90% | 90% |
| OR Quart 2 | 1.8 | 1.00 | 2.6 | 1.1 | 1.6 | 0.89 | 1.5 | 1.0 | 1.6 |
| p Value | 0.067 | 0.99 | 0.0083 | 0.77 | 0.23 | 0.73 | 0.46 | 1.0 | 0.41 |
| 95% CI of | 0.96 | 0.42 | 1.3 | 0.61 | 0.73 | 0.47 | 0.54 | 0.35 | 0.52 |
| OR Quart2 | 3.3 | 2.3 | 5.4 | 2.0 | 3.7 | 1.7 | 3.9 | 2.9 | 5.1 |
| OR Quart 3 | 2.5 | 0.90 | 3.6 | 1.4 | 0.69 | 1.6 | 1.5 | 0.85 | 2.5 |
| p Value | 0.0030 | 0.82 | 3.1E−4 | 0.20 | 0.46 | 0.14 | 0.46 | 0.78 | 0.089 |
| 95% CI of | 1.4 | 0.38 | 1.8 | 0.82 | 0.26 | 0.86 | 0.54 | 0.28 | 0.87 |
| OR Quart3 | 4.5 | 2.2 | 7.3 | 2.5 | 1.8 | 2.8 | 3.9 | 2.6 | 7.4 |
| OR Quart 4 | 3.5 | 1.3 | 6.2 | 2.5 | 1.9 | 3.0 | 3.3 | 1.1 | 5.0 |
| p Value | 2.1E−5 | 0.55 | 1.2E−7 | 8.6E−4 | 0.13 | 7.9E−5 | 0.0081 | 0.80 | 0.0016 |
| 95% CI of | 2.0 | 0.57 | 3.2 | 1.5 | 0.84 | 1.8 | 1.4 | 0.41 | 1.8 |
| OR Quart4 | 6.3 | 2.9 | 12 | 4.2 | 4.1 | 5.3 | 8.1 | 3.2 | 14 |

Immunoglogulin G1

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 2600 | 4230 | 2600 | 4600 | 2600 | 3720 |
| Average | 5310 | 6310 | 5310 | 7320 | 5310 | 5200 |
| Stdev | 7650 | 5510 | 7650 | 9110 | 7650 | 4150 |
| p(t-test) | | 0.18 | | 0.013 | | 0.92 |
| Min | 59.7 | 342 | 59.7 | 138 | 59.7 | 177 |
| Max | 80000 | 28400 | 80000 | 80000 | 80000 | 16300 |
| n (Samp) | 461 | 119 | 461 | 126 | 461 | 47 |
| n (Patient) | 222 | 119 | 222 | 126 | 222 | 47 |
| sCr only | | | | | | |
| Median | 3180 | 5010 | 3180 | 6800 | 3180 | 4830 |
| Average | 5750 | 7340 | 5750 | 8240 | 5750 | 6160 |
| Stdev | 7310 | 6940 | 7310 | 7030 | 7310 | 4600 |
| p(t-test) | | 0.18 | | 0.024 | | 0.78 |
| Min | 3.36 | 342 | 3.36 | 388 | 3.36 | 177 |
| Max | 80000 | 28400 | 80000 | 25600 | 80000 | 14900 |
| n (Samp) | 1011 | 40 | 1011 | 46 | 1011 | 26 |
| n (Patient) | 373 | 40 | 373 | 46 | 373 | 26 |
| UO only | | | | | | |
| Median | 2690 | 5000 | 2690 | 5650 | 2690 | 3900 |
| Average | 5520 | 7480 | 5520 | 8340 | 5520 | 6110 |
| Stdev | 7740 | 6370 | 7740 | 9920 | 7740 | 5690 |
| p(t-test) | | 0.016 | | 0.0012 | | 0.62 |
| Min | 59.7 | 663 | 59.7 | 138 | 59.7 | 550 |
| Max | 80000 | 35700 | 80000 | 80000 | 80000 | 25300 |
| n (Samp) | 433 | 107 | 433 | 115 | 433 | 44 |
| n (Patient) | 171 | 107 | 171 | 115 | 171 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.63 | 0.60 | 0.66 | 0.62 | 0.61 | 0.64 | 0.59 | 0.57 | 0.62 |
| SE | 0.030 | 0.048 | 0.031 | 0.029 | 0.045 | 0.030 | 0.045 | 0.059 | 0.047 |
| p | 1.2E−5 | 0.033 | 2.2E−7 | 7.0E−5 | 0.017 | 2.8E−6 | 0.053 | 0.23 | 0.014 |
| nCohort 1 | 461 | 1011 | 433 | 461 | 1011 | 433 | 461 | 1011 | 433 |
| nCohort 2 | 119 | 40 | 107 | 126 | 46 | 115 | 47 | 26 | 44 |
| Cutoff 1 | 2770 | 3070 | 3090 | 2420 | 2620 | 3070 | 2500 | 2440 | 2880 |
| Sens 1 | 71% | 70% | 70% | 71% | 72% | 70% | 70% | 73% | 70% |
| Spec 1 | 52% | 49% | 55% | 46% | 42% | 55% | 48% | 39% | 52% |
| Cutoff 2 | 2010 | 2840 | 2510 | 1690 | 1550 | 1990 | 1690 | 1690 | 2370 |
| Sens 2 | 81% | 80% | 80% | 80% | 80% | 80% | 81% | 81% | 82% |
| Spec 2 | 39% | 45% | 46% | 31% | 23% | 38% | 31% | 26% | 44% |
| Cutoff 3 | 1390 | 1090 | 1600 | 924 | 911 | 1280 | 1080 | 831 | 1670 |
| Sens 3 | 91% | 90% | 91% | 90% | 91% | 90% | 91% | 92% | 91% |
| Spec 3 | 25% | 14% | 29% | 16% | 12% | 21% | 18% | 10% | 30% |
| Cutoff 4 | 4740 | 5870 | 4990 | 4740 | 5870 | 4990 | 4740 | 5870 | 4990 |
| Sens 4 | 45% | 32% | 50% | 49% | 52% | 53% | 36% | 46% | 36% |
| Spec 4 | 70% | 70% | 70% | 70% | 70% | 70% | 70% | 70% | 70% |
| Cutoff 5 | 7460 | 8580 | 7650 | 7460 | 8580 | 7650 | 7460 | 8580 | 7650 |
| Sens 5 | 29% | 30% | 38% | 33% | 41% | 38% | 28% | 35% | 27% |
| Spec 5 | 80% | 80% | 80% | 80% | 80% | 80% | 80% | 80% | 80% |
| Cutoff 6 | 13400 | 13900 | 14200 | 13400 | 13900 | 14200 | 13400 | 13900 | 14200 |
| Sens 6 | 12% | 18% | 12% | 16% | 22% | 17% | 6% | 4% | 11% |
| Spec 6 | 90% | 90% | 90% | 90% | 90% | 90% | 90% | 90% | 90% |
| OR Quart 2 | 1.9 | 1.3 | 2.0 | 0.94 | 0.69 | 1.4 | 2.5 | 0.80 | 6.0 |
| p Value | 0.085 | 0.59 | 0.090 | 0.85 | 0.46 | 0.37 | 0.088 | 0.74 | 0.022 |
| 95% CI of | 0.92 | 0.46 | 0.90 | 0.48 | 0.26 | 0.68 | 0.87 | 0.21 | 1.3 |
| OR Quart2 | 3.8 | 3.9 | 4.3 | 1.8 | 1.8 | 2.8 | 7.5 | 3.0 | 27 |
| OR Quart 3 | 3.6 | 2.4 | 3.8 | 2.2 | 0.69 | 2.9 | 3.0 | 1.0 | 10 |
| p Value | 1.6E−4 | 0.078 | 3.4E−4 | 0.0077 | 0.46 | 0.0015 | 0.039 | 1.0 | 0.0020 |
| 95% CI of | 1.8 | 0.91 | 1.8 | 1.2 | 0.26 | 1.5 | 1.1 | 0.29 | 2.4 |
| OR Quart3 | 6.9 | 6.3 | 7.9 | 4.0 | 1.8 | 5.6 | 8.7 | 3.5 | 46 |
| OR Quart 4 | 3.7 | 2.0 | 5.1 | 2.6 | 2.3 | 3.8 | 3.5 | 2.5 | 7.1 |
| p Value | 1.0E−4 | 0.16 | 8.4E−6 | 0.0011 | 0.034 | 4.2E−5 | 0.017 | 0.096 | 0.011 |
| 95% CI of | 1.9 | 0.75 | 2.5 | 1.5 | 1.1 | 2.0 | 1.2 | 0.85 | 1.6 |
| OR Quart4 | 7.1 | 5.5 | 10 | 4.7 | 5.0 | 7.3 | 9.9 | 7.1 | 32 |

Immunoglogulin G2

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 7990 | 11000 | 7990 | 12100 | 7990 | 9560 |
| Average | 16000 | 20900 | 16000 | 26500 | 16000 | 16600 |
| Stdev | 26200 | 31200 | 26200 | 45400 | 26200 | 26800 |
| p(t-test) | | 0.077 | | 8.8E−4 | | 0.87 |
| Min | 119 | 819 | 119 | 640 | 119 | 1050 |
| Max | 240000 | 240000 | 240000 | 240000 | 240000 | 172000 |
| n (Samp) | 461 | 119 | 461 | 126 | 461 | 47 |
| n (Patient) | 222 | 119 | 222 | 126 | 222 | 47 |
| sCr only | | | | | | |
| Median | 9050 | 11500 | 9050 | 13400 | 9050 | 9970 |
| Average | 17700 | 24000 | 17700 | 35800 | 17700 | 22000 |
| Stdev | 29600 | 43100 | 29600 | 55000 | 29600 | 34000 |
| p(t-test) | | 0.19 | | 1.1E−4 | | 0.46 |
| Min | 119 | 819 | 119 | 1070 | 119 | 1310 |
| Max | 240000 | 240000 | 240000 | 240000 | 240000 | 172000 |
| n (Samp) | 1011 | 40 | 1011 | 46 | 1011 | 26 |
| n (Patient) | 373 | 40 | 373 | 46 | 373 | 26 |
| UO only | | | | | | |
| Median | 8540 | 13200 | 8540 | 13500 | 8540 | 10800 |
| Average | 17400 | 26300 | 17400 | 29800 | 17400 | 21300 |
| Stdev | 29900 | 38600 | 29900 | 50500 | 29900 | 38200 |
| p(t-test) | | 0.010 | | 8.8E−4 | | 0.43 |
| Min | 334 | 2930 | 334 | 640 | 334 | 1050 |
| Max | 240000 | 240000 | 240000 | 240000 | 240000 | 240000 |
| n (Samp) | 433 | 107 | 433 | 115 | 433 | 44 |
| n (Patient) | 171 | 107 | 171 | 115 | 171 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.60 | 0.55 | 0.63 | 0.59 | 0.58 | 0.61 | 0.54 | 0.56 | 0.56 |
| SE | 0.030 | 0.048 | 0.031 | 0.029 | 0.045 | 0.031 | 0.045 | 0.059 | 0.047 |
| p | 0.0016 | 0.26 | 2.4E−5 | 0.0022 | 0.064 | 4.8E−4 | 0.40 | 0.30 | 0.21 |
| nCohort 1 | 461 | 1011 | 433 | 461 | 1011 | 433 | 461 | 1011 | 433 |
| nCohort 2 | 119 | 40 | 107 | 126 | 46 | 115 | 47 | 26 | 44 |
| Cutoff 1 | 7640 | 7760 | 8630 | 6690 | 5500 | 7670 | 6460 | 6690 | 7030 |
| Sens 1 | 71% | 70% | 70% | 71% | 72% | 70% | 70% | 73% | 70% |
| Spec 1 | 48% | 43% | 51% | 42% | 59% | 45% | 41% | 37% | 42% |
| Cutoff 2 | 5300 | 6310 | 6460 | 4860 | 4630 | 5930 | 4240 | 6520 | 4680 |
| Sens 2 | 81% | 80% | 80% | 80% | 80% | 80% | 81% | 81% | 82% |
| Spec 2 | 33% | 34% | 38% | 29% | 23% | 33% | 25% | 36% | 24% |
| Cutoff 3 | 3890 | 2220 | 4750 | 2560 | 3170 | 3280 | 2800 | 3420 | 3060 |
| Sens 3 | 91% | 90% | 91% | 90% | 91% | 91% | 91% | 92% | 91% |
| Spec 3 | 21% | 9% | 24% | 12% | 13% | 14% | 14% | 15% | 13% |
| Cutoff 4 | 14400 | 15000 | 14500 | 14400 | 15000 | 14500 | 14400 | 15000 | 14500 |
| Sens 4 | 36% | 30% | 44% | 43% | 43% | 45% | 34% | 42% | 36% |
| Spec 4 | 70% | 70% | 70% | 70% | 70% | 70% | 70% | 70% | 70% |
| Cutoff 5 | 19800 | 21300 | 19800 | 19800 | 21300 | 19800 | 19800 | 21300 | 19800 |
| Sens 5 | 25% | 25% | 33% | 31% | 37% | 33% | 21% | 27% | 23% |
| Spec 5 | 80% | 80% | 80% | 80% | 80% | 80% | 80% | 80% | 80% |
| Cutoff 6 | 32800 | 36600 | 35300 | 32800 | 36600 | 35300 | 32800 | 36600 | 35300 |
| Sens 6 | 16% | 15% | 20% | 18% | 26% | 17% | 9% | 12% | 11% |
| Spec 6 | 90% | 90% | 90% | 90% | 90% | 90% | 90% | 90% | 90% |
| OR Quart 2 | 1.4 | 0.87 | 1.7 | 1.1 | 0.63 | 1.3 | 0.89 | 1.4 | 0.88 |
| p Value | 0.32 | 0.79 | 0.16 | 0.77 | 0.34 | 0.41 | 0.81 | 0.56 | 0.80 |
| 95% CI of | 0.72 | 0.31 | 0.82 | 0.59 | 0.24 | 0.69 | 0.35 | 0.44 | 0.33 |
| OR Quart2 | 2.7 | 2.4 | 3.4 | 2.0 | 1.6 | 2.5 | 2.3 | 4.5 | 2.4 |
| OR Quart 3 | 3.0 | 1.8 | 2.9 | 1.7 | 1.0 | 1.9 | 1.7 | 1.0 | 1.6 |
| p Value | 4.5E−4 | 0.20 | 0.0020 | 0.064 | 1.0 | 0.046 | 0.22 | 1.0 | 0.28 |
| 95% CI of | 1.6 | 0.74 | 1.5 | 0.97 | 0.43 | 1.0 | 0.73 | 0.29 | 0.68 |
| OR Quart3 | 5.5 | 4.3 | 5.7 | 3.1 | 2.3 | 3.5 | 3.9 | 3.5 | 3.9 |
| OR Quart 4 | 2.2 | 1.4 | 3.3 | 2.1 | 1.6 | 2.6 | 1.2 | 1.8 | 1.5 |
| p Value | 0.016 | 0.49 | 5.4E−4 | 0.0091 | 0.25 | 0.0025 | 0.66 | 0.29 | 0.38 |
| 95% CI of | 1.2 | 0.55 | 1.7 | 1.2 | 0.72 | 1.4 | 0.51 | 0.60 | 0.61 |
| OR Quart4 | 4.0 | 3.5 | 6.4 | 3.8 | 3.4 | 4.7 | 2.9 | 5.5 | 3.6 |

Interleukin-11

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 109 | 223 | 109 | 165 | 109 | 120 |
| Average | 186 | 304 | 186 | 233 | 186 | 193 |
| Stdev | 225 | 268 | 225 | 205 | 225 | 328 |
| p(t-test) | | 1.2E−6 | | 0.033 | | 0.83 |
| Min | 0.154 | 13.2 | 0.154 | 7.35 | 0.154 | 0.0822 |
| Max | 1980 | 1780 | 1980 | 1050 | 1980 | 2260 |
| n (Samp) | 461 | 119 | 461 | 129 | 461 | 47 |
| n (Patient) | 223 | 119 | 223 | 129 | 223 | 47 |
| sCr only | | | | | | |
| Median | 131 | 226 | 131 | 202 | 131 | 124 |
| Average | 216 | 253 | 216 | 240 | 216 | 167 |
| Stdev | 249 | 173 | 249 | 191 | 249 | 139 |
| p(t-test) | | 0.35 | | 0.52 | | 0.32 |
| Min | 0.0822 | 13.2 | 0.0822 | 7.35 | 0.0822 | 0.0822 |
| Max | 2260 | 659 | 2260 | 996 | 2260 | 558 |
| n (Samp) | 1017 | 40 | 1017 | 46 | 1017 | 26 |
| n (Patient) | 375 | 40 | 375 | 46 | 375 | 26 |
| UO only | | | | | | |
| Median | 131 | 232 | 131 | 169 | 131 | 138 |
| Average | 206 | 349 | 206 | 264 | 206 | 244 |
| Stdev | 234 | 381 | 234 | 315 | 234 | 448 |
| p(t-test) | | 1.1E-6 | | 0.026 | | 0.35 |
| Min | 2.83 | 41.8 | 2.83 | 8.36 | 2.83 | 14.5 |
| Max | 1980 | 2900 | 1980 | 2590 | 1980 | 2260 |
| n (Samp) | 434 | 107 | 434 | 118 | 434 | 44 |
| n (Patient) | 173 | 107 | 173 | 118 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.70 | 0.61 | 0.69 | 0.62 | 0.58 | 0.59 | 0.52 | 0.47 | 0.48 |
| SE | 0.029 | 0.048 | 0.031 | 0.029 | 0.045 | 0.030 | 0.045 | 0.058 | 0.046 |
| p | 6.9E−12 | 0.029 | 1.2E−9 | 7.4E−5 | 0.072 | 0.0033 | 0.69 | 0.62 | 0.68 |
| nCohort 1 | 461 | 1017 | 434 | 461 | 1017 | 434 | 461 | 1017 | 434 |
| nCohort 2 | 119 | 40 | 107 | 129 | 46 | 118 | 47 | 26 | 44 |
| Cutoff 1 | 144 | 111 | 152 | 106 | 105 | 111 | 79.8 | 66.2 | 79.8 |
| Sens 1 | 71% | 70% | 70% | 71% | 72% | 70% | 70% | 73% | 70% |
| Spec 1 | 61% | 43% | 56% | 48% | 40% | 44% | 36% | 23% | 31% |
| Cutoff 2 | 107 | 78.7 | 121 | 76.0 | 76.5 | 85.7 | 66.1 | 57.1 | 66.1 |
| Sens 2 | 81% | 80% | 80% | 81% | 80% | 81% | 81% | 81% | 82% |
| Spec 2 | 49% | 29% | 47% | 34% | 28% | 32% | 30% | 19% | 24% |
| Cutoff 3 | 73.6 | 41.6 | 87.1 | 55.8 | 40.7 | 66.2 | 53.9 | 41.4 | 31.8 |
| Sens 3 | 91% | 90% | 91% | 91% | 91% | 91% | 91% | 92% | 91% |
| Spec 3 | 33% | 10% | 33% | 25% | 10% | 24% | 23% | 10% | 6% |
| Cutoff 4 | 189 | 218 | 217 | 189 | 218 | 217 | 189 | 218 | 217 |
| Sens 4 | 58% | 55% | 52% | 43% | 46% | 37% | 21% | 27% | 23% |
| Spec 4 | 70% | 70% | 70% | 70% | 70% | 70% | 70% | 70% | 70% |
| Cutoff 5 | 254 | 308 | 289 | 254 | 308 | 289 | 254 | 308 | 289 |
| Sens 5 | 46% | 35% | 42% | 31% | 30% | 28% | 17% | 15% | 18% |
| Spec 5 | 80% | 80% | 80% | 80% | 80% | 80% | 80% | 80% | 80% |
| Cutoff 6 | 399 | 503 | 414 | 399 | 503 | 414 | 399 | 503 | 414 |
| Sens 6 | 27% | 8% | 28% | 15% | 7% | 15% | 6% | 4% | 7% |
| Spec 6 | 90% | 90% | 90% | 90% | 90% | 90% | 90% | 90% | 90% |
| OR Quart 2 | 2.7 | 1.6 | 2.6 | 2.3 | 1.6 | 1.8 | 2.5 | 1.6 | 1.6 |
| p Value | 0.017 | 0.40 | 0.024 | 0.013 | 0.35 | 0.081 | 0.055 | 0.40 | 0.27 |
| 95% CI of | 1.2 | 0.52 | 1.1 | 1.2 | 0.61 | 0.93 | 0.98 | 0.52 | 0.68 |
| OR Quart2 | 6.1 | 5.0 | 5.9 | 4.5 | 4.2 | 3.4 | 6.2 | 5.0 | 4.0 |
| OR Quart 3 | 4.6 | 1.8 | 4.2 | 3.3 | 1.3 | 2.3 | 2.1 | 1.0 | 1.4 |
| p Value | 1.1E−4 | 0.29 | 3.7E−4 | 3.0E−4 | 0.62 | 0.011 | 0.12 | 1.0 | 0.49 |
| 95% CI of | 2.1 | 0.60 | 1.9 | 1.7 | 0.47 | 1.2 | 0.83 | 0.29 | 0.55 |
| OR Quart3 | 10 | 5.5 | 9.2 | 6.3 | 3.5 | 4.2 | 5.5 | 3.5 | 3.4 |
| OR Quart 4 | 9.0 | 3.8 | 7.2 | 3.6 | 2.8 | 2.4 | 1.5 | 1.6 | 1.0 |
| p Value | 1.2E−8 | 0.0097 | 4.4E−7 | 8.9E−5 | 0.021 | 0.0072 | 0.45 | 0.40 | 0.99 |
| 95% CI of | 4.2 | 1.4 | 3.3 | 1.9 | 1.2 | 1.3 | 0.54 | 0.52 | 0.39 |
| OR Quart4 | 19 | 10 | 15 | 6.8 | 6.9 | 4.4 | 4.0 | 5.0 | 2.6 |

Interleukin-2 receptor alpha chain

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 434 | 788 | 434 | 572 | 434 | 526 |
| Average | 833 | 1150 | 833 | 1190 | 833 | 986 |
| Stdev | 1060 | 1220 | 1060 | 1640 | 1060 | 1440 |
| p(t-test) | | 0.0055 | | 0.0028 | | 0.37 |
| Min | 0.0317 | 0.0339 | 0.0317 | 0.0339 | 0.0317 | 0.120 |
| Max | 10400 | 6080 | 10400 | 9090 | 10400 | 7740 |
| n (Samp) | 462 | 120 | 462 | 130 | 462 | 47 |
| n (Patient) | 223 | 120 | 223 | 130 | 223 | 47 |
| sCr only | | | | | | |
| Median | 622 | 431 | 622 | 570 | 622 | 515 |
| Average | 1060 | 1110 | 1060 | 1190 | 1060 | 1020 |
| Stdev | 1380 | 1720 | 1380 | 1550 | 1380 | 1450 |
| p(t-test) | | 0.82 | | 0.56 | | 0.87 |
| Min | 0.0317 | 0.0339 | 0.0317 | 0.0365 | 0.0317 | 0.141 |
| Max | 10400 | 7330 | 10400 | 7190 | 10400 | 5640 |
| n (Samp) | 1019 | 40 | 1019 | 46 | 1019 | 26 |
| n (Patient) | 375 | 40 | 375 | 46 | 375 | 26 |
| UO only | | | | | | |
| Median | 454 | 942 | 454 | 734 | 454 | 533 |
| Average | 840 | 1310 | 840 | 1330 | 840 | 981 |
| Stdev | 1070 | 1260 | 1070 | 1690 | 1070 | 1430 |
| p(t-test) | | 8.8E−5 | | 1.3E−4 | | 0.42 |
| Min | 0.0317 | 0.0339 | 0.0317 | 0.0339 | 0.0317 | 0.120 |
| Max | 10400 | 6080 | 10400 | 9090 | 10400 | 7740 |
| n (Samp) | 436 | 108 | 436 | 119 | 436 | 44 |
| n (Patient) | 173 | 108 | 173 | 119 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.60 | 0.44 | 0.65 | 0.54 | 0.50 | 0.58 | 0.52 | 0.47 | 0.52 |
| SE | 0.030 | 0.048 | 0.031 | 0.029 | 0.044 | 0.030 | 0.045 | 0.058 | 0.046 |
| p | 9.8E−4 | 0.19 | 1.9E−6 | 0.14 | 0.93 | 0.0089 | 0.64 | 0.58 | 0.66 |
| nCohort 1 | 462 | 1019 | 436 | 462 | 1019 | 436 | 462 | 1019 | 436 |
| nCohort 2 | 120 | 40 | 108 | 130 | 46 | 119 | 47 | 26 | 44 |
| Cutoff 1 | 394 | 73.3 | 548 | 216 | 226 | 280 | 213 | 195 | 205 |
| Sens 1 | 70% | 70% | 70% | 70% | 72% | 71% | 70% | 73% | 70% |
| Spec 1 | 48% | 13% | 55% | 34% | 27% | 37% | 34% | 24% | 31% |
| Cutoff 2 | 228 | 38.4 | 342 | 139 | 148 | 152 | 139 | 158 | 135 |
| Sens 2 | 80% | 80% | 81% | 80% | 80% | 81% | 81% | 81% | 82% |
| Spec 2 | 35% | 9% | 42% | 25% | 20% | 26% | 25% | 21% | 23% |
| Cutoff 3 | 55.0 | 23.9 | 128 | 33.7 | 35.6 | 49.3 | 45.1 | 126 | 40.9 |
| Sens 3 | 90% | 90% | 91% | 90% | 91% | 91% | 91% | 92% | 91% |
| Spec 3 | 16% | 8% | 23% | 13% | 9% | 14% | 14% | 17% | 12% |
| Cutoff 4 | 932 | 1150 | 928 | 932 | 1150 | 928 | 932 | 1150 | 928 |
| Sens 4 | 46% | 30% | 51% | 37% | 35% | 42% | 34% | 19% | 34% |
| Spec 4 | 70% | 70% | 70% | 70% | 70% | 70% | 70% | 70% | 70% |
| Cutoff 5 | 1410 | 1690 | 1330 | 1410 | 1690 | 1330 | 1410 | 1690 | 1330 |
| Sens 5 | 24% | 18% | 31% | 25% | 24% | 31% | 19% | 15% | 23% |
| Spec 5 | 80% | 80% | 80% | 80% | 80% | 80% | 80% | 80% | 80% |
| Cutoff 6 | 2100 | 2510 | 2210 | 2100 | 2510 | 2210 | 2100 | 2510 | 2210 |
| Sens 6 | 15% | 15% | 18% | 16% | 15% | 18% | 11% | 12% | 11% |
| Spec 6 | 90% | 90% | 90% | 90% | 90% | 90% | 90% | 90% | 90% |
| OR Quart 2 | 0.89 | 0.49 | 1.7 | 0.88 | 0.74 | 0.94 | 1.1 | 1.2 | 1.1 |
| p Value | 0.72 | 0.20 | 0.19 | 0.66 | 0.51 | 0.85 | 0.82 | 0.76 | 0.81 |
| 95% CI of | 0.46 | 0.17 | 0.78 | 0.50 | 0.31 | 0.51 | 0.45 | 0.36 | 0.44 |
| OR Quart2 | 1.7 | 1.5 | 3.6 | 1.6 | 1.8 | 1.7 | 2.7 | 4.0 | 2.9 |
| OR Quart 3 | 2.1 | 0.90 | 4.0 | 1.1 | 1.0 | 1.2 | 1.3 | 1.4 | 1.5 |
| p Value | 0.011 | 0.82 | 1.0E−4 | 0.78 | 0.99 | 0.47 | 0.51 | 0.56 | 0.37 |
| 95% CI of | 1.2 | 0.36 | 2.0 | 0.62 | 0.44 | 0.69 | 0.56 | 0.44 | 0.62 |
| OR Quart3 | 3.8 | 2.2 | 8.1 | 1.9 | 2.3 | 2.2 | 3.2 | 4.5 | 3.7 |
| OR Quart 4 | 2.0 | 1.6 | 4.2 | 1.3 | 1.1 | 1.8 | 1.3 | 1.6 | 1.4 |
| p Value | 0.023 | 0.23 | 6.6E−5 | 0.34 | 0.83 | 0.038 | 0.53 | 0.40 | 0.49 |
| 95% CI of | 1.1 | 0.73 | 2.1 | 0.76 | 0.49 | 1.0 | 0.56 | 0.52 | 0.55 |
| OR Quart4 | 3.5 | 3.7 | 8.4 | 2.2 | 2.4 | 3.2 | 3.1 | 5.0 | 3.4 |

Neutrophil collagenase

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 2540 | 4730 | 2540 | 5520 | 2540 | 3780 |
| Average | 11100 | 18100 | 11100 | 19900 | 11100 | 16200 |
| Stdev | 40300 | 56700 | 40300 | 45400 | 40300 | 36300 |
| p(t-test) | | 0.099 | | 0.028 | | 0.41 |
| Min | 0.114 | 150 | 0.114 | 22.5 | 0.114 | 50.0 |
| Max | 670000 | 625000 | 670000 | 312000 | 670000 | 236000 |
| n (Samp) | 498 | 142 | 498 | 140 | 498 | 48 |
| n (Patient) | 235 | 142 | 235 | 140 | 235 | 48 |
| sCr only | | | | | | |
| Median | 3460 | 3530 | 3460 | 7850 | 3460 | 4980 |
| Average | 15100 | 14000 | 15100 | 36300 | 15100 | 16200 |
| Stdev | 44100 | 25900 | 44100 | 106000 | 44100 | 44400 |
| p(t-test) | | 0.86 | | 0.0024 | | 0.90 |
| Min | 0.114 | 150 | 0.114 | 40.5 | 0.114 | 111 |
| Max | 670000 | 128000 | 670000 | 625000 | 670000 | 236000 |
| n (Samp) | 1095 | 46 | 1095 | 51 | 1095 | 28 |
| n (Patient) | 398 | 46 | 398 | 51 | 398 | 28 |
| UO only | | | | | | |
| Median | 2550 | 5870 | 2550 | 6090 | 2550 | 4960 |
| Average | 8970 | 26600 | 8970 | 24800 | 8970 | 11700 |
| Stdev | 22700 | 78300 | 22700 | 53900 | 22700 | 16600 |
| p(t-test) | | 1.7E−5 | | 6.8E−7 | | 0.42 |
| Min | 0.114 | 0.260 | 0.114 | 22.5 | 0.114 | 50.0 |
| Max | 300000 | 625000 | 300000 | 327000 | 300000 | 66900 |
| n (Samp) | 485 | 129 | 485 | 130 | 485 | 47 |
| n (Patient) | 190 | 129 | 190 | 130 | 190 | 47 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.62 | 0.54 | 0.65 | 0.61 | 0.57 | 0.64 | 0.58 | 0.54 | 0.59 |
| SE | 0.028 | 0.044 | 0.029 | 0.028 | 0.043 | 0.029 | 0.045 | 0.056 | 0.045 |
| p | 2.5E−5 | 0.33 | 1.5E−7 | 6.4E−5 | 0.091 | 9.6E−7 | 0.075 | 0.51 | 0.055 |
| nCohort 1 | 498 | 1095 | 485 | 498 | 1095 | 485 | 498 | 1095 | 485 |
| nCohort 2 | 142 | 46 | 129 | 140 | 51 | 130 | 48 | 28 | 47 |
| Cutoff 1 | 2030 | 1700 | 2620 | 2250 | 1970 | 2450 | 1430 | 1820 | 1550 |
| Sens 1 | 70% | 72% | 71% | 70% | 71% | 70% | 71% | 71% | 70% |
| Spec 1 | 44% | 34% | 51% | 47% | 37% | 49% | 37% | 36% | 38% |
| Cutoff 2 | 1110 | 977 | 1710 | 1060 | 935 | 1460 | 870 | 1410 | 898 |
| Sens 2 | 80% | 80% | 81% | 80% | 80% | 80% | 81% | 82% | 81% |
| Spec 2 | 31% | 22% | 40% | 30% | 21% | 36% | 27% | 29% | 26% |
| Cutoff 3 | 765 | 418 | 844 | 536 | 546 | 804 | 191 | 365 | 387 |
| Sens 3 | 90% | 91% | 91% | 90% | 90% | 90% | 92% | 93% | 91% |
| Spec 3 | 25% | 11% | 25% | 19% | 14% | 25% | 7% | 10% | 13% |
| Cutoff 4 | 6260 | 8700 | 6150 | 6260 | 8700 | 6150 | 6260 | 8700 | 6150 |
| Sens 4 | 45% | 41% | 49% | 46% | 49% | 50% | 44% | 32% | 47% |
| Spec 4 | 70% | 70% | 70% | 70% | 70% | 70% | 70% | 70% | 70% |
| Cutoff 5 | 10000 | 14900 | 9570 | 10000 | 14900 | 9570 | 10000 | 14900 | 9570 |
| Sens 5 | 35% | 28% | 39% | 35% | 27% | 38% | 35% | 18% | 34% |
| Spec 5 | 80% | 80% | 80% | 80% | 80% | 80% | 80% | 80% | 80% |
| Cutoff 6 | 20800 | 32400 | 21900 | 20800 | 32400 | 21900 | 20800 | 32400 | 21900 |
| Sens 6 | 16% | 7% | 20% | 19% | 14% | 20% | 21% | 7% | 15% |
| Spec 6 | 90% | 90% | 90% | 90% | 90% | 90% | 90% | 90% | 90% |
| OR Quart 2 | 1.4 | 0.74 | 2.1 | 1.3 | 0.52 | 1.8 | 1.1 | 2.0 | 1.1 |
| p Value | 0.29 | 0.51 | 0.026 | 0.46 | 0.18 | 0.083 | 0.82 | 0.25 | 0.81 |
| 95% CI of | 0.76 | 0.31 | 1.1 | 0.69 | 0.21 | 0.93 | 0.44 | 0.60 | 0.44 |
| OR Quart2 | 2.5 | 1.8 | 4.2 | 2.3 | 1.3 | 3.4 | 2.8 | 6.8 | 2.9 |
| OR Quart 3 | 2.1 | 0.66 | 2.6 | 2.0 | 1.0 | 2.4 | 1.2 | 2.5 | 1.2 |
| p Value | 0.012 | 0.37 | 0.0038 | 0.018 | 1.0 | 0.0053 | 0.64 | 0.12 | 0.64 |
| 95% CI of | 1.2 | 0.26 | 1.4 | 1.1 | 0.46 | 1.3 | 0.50 | 0.79 | 0.50 |
| OR Quart3 | 3.7 | 1.6 | 5.1 | 3.5 | 2.2 | 4.6 | 3.1 | 8.2 | 3.1 |
| OR Quart 4 | 2.9 | 1.4 | 4.6 | 2.6 | 1.4 | 3.7 | 2.1 | 1.5 | 2.0 |
| p Value | 1.6E−4 | 0.35 | 2.4E−6 | 7.1E−4 | 0.36 | 2.1E−5 | 0.076 | 0.53 | 0.10 |
| 95% CI of | 1.7 | 0.67 | 2.4 | 1.5 | 0.68 | 2.0 | 0.92 | 0.42 | 0.87 |
| OR Quart4 | 5.1 | 3.1 | 8.6 | 4.5 | 2.9 | 6.9 | 4.9 | 5.4 | 4.7 |

Protransforming growth factor alpha

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 5.17 | 7.34 | 5.17 | 8.58 | 5.17 | 5.71 |
| Average | 10.2 | 12.2 | 10.2 | 11.9 | 10.2 | 7.65 |
| Stdev | 23.4 | 16.8 | 23.4 | 11.0 | 23.4 | 7.10 |
| p(t-test) | | 0.38 | | 0.43 | | 0.46 |
| Min | 0.00184 | 0.00574 | 0.00184 | 0.00603 | 0.00184 | 0.00440 |
| Max | 361 | 135 | 361 | 54.1 | 361 | 32.4 |
| n (Samp) | 462 | 116 | 462 | 130 | 462 | 47 |
| n (Patient) | 223 | 116 | 223 | 130 | 223 | 47 |
| sCr only | | | | | | |
| Median | 6.18 | 7.25 | 6.18 | 7.37 | 6.18 | 4.95 |
| Average | 10.8 | 9.21 | 10.8 | 10.0 | 10.8 | 6.94 |
| Stdev | 19.7 | 7.72 | 19.7 | 10.1 | 19.7 | 6.70 |
| p(t-test) | | 0.61 | | 0.79 | | 0.32 |
| Min | 0.00184 | 0.00574 | 0.00184 | 0.00574 | 0.00184 | 0.00574 |
| Max | 361 | 30.2 | 361 | 50.4 | 361 | 27.7 |
| n (Samp) | 1014 | 39 | 1014 | 46 | 1014 | 26 |
| n (Patient) | 375 | 39 | 375 | 46 | 375 | 26 |
| UO only | | | | | | |
| Median | 5.14 | 8.56 | 5.14 | 10.2 | 5.14 | 6.78 |
| Average | 10.3 | 14.4 | 10.3 | 13.6 | 10.3 | 8.83 |
| Stdev | 24.0 | 18.7 | 24.0 | 12.8 | 24.0 | 7.87 |
| p(t-test) | | 0.10 | | 0.15 | | 0.68 |
| Min | 0.00184 | 0.00603 | 0.00184 | 0.00603 | 0.00184 | 0.00440 |
| Max | 361 | 135 | 361 | 77.6 | 361 | 36.0 |
| n (Samp) | 436 | 104 | 436 | 119 | 436 | 44 |
| n (Patient) | 173 | 104 | 173 | 119 | 173 | 44 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.59 | 0.52 | 0.64 | 0.63 | 0.52 | 0.67 | 0.52 | 0.43 | 0.55 |
| SE | 0.030 | 0.048 | 0.032 | 0.029 | 0.044 | 0.030 | 0.045 | 0.059 | 0.047 |
| p | 0.0022 | 0.67 | 1.7E−5 | 5.1E−6 | 0.61 | 2.5E−8 | 0.73 | 0.25 | 0.24 |
| nCohort 1 | 462 | 1014 | 436 | 462 | 1014 | 436 | 462 | 1014 | 436 |
| nCohort 2 | 116 | 39 | 104 | 130 | 46 | 119 | 47 | 26 | 44 |
| Cutoff 1 | 4.08 | 3.95 | 5.24 | 5.35 | 3.53 | 6.12 | 2.73 | 2.73 | 3.27 |
| Sens 1 | 71% | 72% | 70% | 70% | 72% | 71% | 70% | 73% | 70% |
| Spec 1 | 41% | 32% | 51% | 52% | 29% | 56% | 29% | 22% | 32% |
| Cutoff 2 | 3.05 | 2.30 | 3.71 | 3.25 | 3.07 | 3.61 | 2.55 | 2.17 | 2.52 |
| Sens 2 | 80% | 82% | 81% | 80% | 80% | 81% | 81% | 81% | 82% |
| Spec 2 | 32% | 18% | 36% | 34% | 25% | 35% | 27% | 17% | 23% |
| Cutoff 3 | 2.14 | 0.804 | 2.37 | 2.15 | 0.00713 | 2.17 | 1.65 | 1.60 | 1.79 |
| Sens 3 | 91% | 92% | 90% | 90% | 91% | 92% | 91% | 92% | 91% |
| Spec 3 | 23% | 6% | 22% | 23% | 2% | 21% | 18% | 12% | 18% |
| Cutoff 4 | 8.54 | 9.97 | 8.15 | 8.54 | 9.97 | 8.15 | 8.54 | 9.97 | 8.15 |
| Sens 4 | 43% | 41% | 51% | 51% | 33% | 61% | 32% | 27% | 41% |
| Spec 4 | 70% | 70% | 70% | 70% | 70% | 70% | 70% | 70% | 70% |
| Cutoff 5 | 11.8 | 13.4 | 11.2 | 11.8 | 13.4 | 11.2 | 11.8 | 13.4 | 11.2 |
| Sens 5 | 30% | 23% | 38% | 36% | 28% | 45% | 21% | 12% | 30% |
| Spec 5 | 80% | 80% | 80% | 80% | 80% | 80% | 80% | 80% | 80% |
| Cutoff 6 | 19.3 | 22.6 | 19.8 | 19.3 | 22.6 | 19.8 | 19.3 | 22.6 | 19.8 |
| Sens 6 | 17% | 8% | 20% | 16% | 9% | 20% | 6% | 4% | 7% |
| Spec 6 | 90% | 90% | 90% | 90% | 90% | 90% | 90% | 90% | 90% |
| OR Quart 2 | 1.8 | 0.69 | 1.9 | 1.2 | 0.81 | 1.1 | 1.6 | 1.0 | 0.56 |
| p Value | 0.064 | 0.46 | 0.077 | 0.51 | 0.65 | 0.87 | 0.28 | 1.0 | 0.24 |
| 95% CI of | 0.97 | 0.26 | 0.93 | 0.65 | 0.33 | 0.52 | 0.68 | 0.29 | 0.21 |
| OR Quart2 | 3.5 | 1.8 | 4.0 | 2.4 | 2.0 | 2.2 | 3.9 | 3.5 | 1.5 |
| OR Quart 3 | 1.7 | 1.0 | 2.5 | 2.4 | 1.0 | 2.1 | 1.2 | 1.6 | 0.73 |
| p Value | 0.11 | 1.0 | 0.013 | 0.0040 | 1.0 | 0.021 | 0.64 | 0.40 | 0.49 |
| 95% CI of | 0.89 | 0.41 | 1.2 | 1.3 | 0.43 | 1.1 | 0.50 | 0.52 | 0.30 |
| OR Quart3 | 3.2 | 2.4 | 5.0 | 4.4 | 2.3 | 4.0 | 3.1 | 5.0 | 1.8 |
| OR Quart 4 | 2.7 | 1.2 | 4.0 | 3.4 | 1.4 | 4.3 | 1.5 | 1.6 | 1.4 |
| p Value | 0.0017 | 0.67 | 7.6E−5 | 5.8E−5 | 0.42 | 3.7E−6 | 0.39 | 0.40 | 0.42 |
| 95% CI of | 1.4 | 0.51 | 2.0 | 1.9 | 0.62 | 2.3 | 0.61 | 0.52 | 0.62 |
| OR Quart4 | 4.9 | 2.8 | 7.8 | 6.1 | 3.1 | 7.9 | 3.6 | 5.0 | 3.1 |

CA 15-3

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 423 | 434 | 423 | 434 | 423 | 434 |
| Average | 280 | 376 | 280 | 378 | 280 | 361 |
| Stdev | 244 | 215 | 244 | 212 | 244 | 141 |
| p(t-test) | | 0.0018 | | 0.0022 | | 0.098 |
| Min | 3.03 | 11.6 | 3.03 | 6.39 | 3.03 | 21.6 |
| Max | 784 | 784 | 784 | 784 | 784 | 434 |
| n (Samp) | 194 | 86 | 194 | 76 | 194 | 26 |
| n (Patient) | 123 | 86 | 123 | 76 | 123 | 26 |
| sCr only | | | | | | |
| Median | 434 | 434 | 434 | 434 | 434 | 434 |
| Average | 331 | 306 | 331 | 360 | 331 | 296 |
| Stdev | 227 | 228 | 227 | 202 | 227 | 186 |
| p(t-test) | | 0.59 | | 0.50 | | 0.55 |
| Min | 1.17 | 9.20 | 1.17 | 20.6 | 1.17 | 11.7 |
| Max | 784 | 784 | 784 | 784 | 784 | 434 |
| n (Samp) | 428 | 26 | 428 | 28 | 428 | 16 |
| n (Patient) | 210 | 26 | 210 | 28 | 210 | 16 |
| UO only | | | | | | |
| Median | 434 | 434 | 434 | 434 | 434 | 434 |
| Average | 286 | 395 | 286 | 377 | 286 | 352 |
| Stdev | 233 | 193 | 233 | 216 | 233 | 147 |
| p(t-test) | | 2.9E−4 | | 0.0041 | | 0.16 |
| Min | 3.03 | 12.2 | 3.03 | 6.39 | 3.03 | 28.4 |
| Max | 784 | 784 | 784 | 784 | 784 | 434 |
| n (Samp) | 210 | 78 | 210 | 72 | 210 | 27 |
| n (Patient) | 119 | 78 | 119 | 72 | 119 | 27 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.62 | 0.47 | 0.63 | 0.63 | 0.55 | 0.62 | 0.62 | 0.48 | 0.60 |
| SE | 0.037 | 0.059 | 0.038 | 0.039 | 0.058 | 0.040 | 0.062 | 0.074 | 0.061 |
| p | 0.0010 | 0.64 | 5.3E−4 | 0.0013 | 0.42 | 0.0027 | 0.056 | 0.81 | 0.099 |
| nCohort 1 | 194 | 428 | 210 | 194 | 428 | 210 | 194 | 428 | 210 |
| nCohort 2 | 86 | 26 | 78 | 76 | 28 | 72 | 26 | 16 | 27 |
| Cutoff 1 | 320 | 66.6 | 427 | 413 | 413 | 427 | 413 | 113 | 427 |
| Sens 1 | 71% | 73% | 74% | 72% | 71% | 71% | 73% | 75% | 70% |
| Spec 1 | 47% | 25% | 47% | 50% | 39% | 47% | 50% | 30% | 47% |
| Cutoff 2 | 123 | 52.6 | 253 | 129 | 88.9 | 64.1 | 248 | 62.4 | 239 |
| Sens 2 | 80% | 81% | 81% | 80% | 82% | 81% | 81% | 81% | 81% |
| Spec 2 | 44% | 23% | 45% | 45% | 28% | 34% | 47% | 24% | 44% |
| Cutoff 3 | 48.4 | 25.1 | 52.2 | 33.2 | 24.5 | 42.9 | 68.6 | 21.3 | 68.6 |
| Sens 3 | 91% | 92% | 91% | 91% | 93% | 90% | 92% | 94% | 93% |
| Spec 3 | 33% | 13% | 32% | 25% | 12% | 28% | 39% | 11% | 35% |
| Cutoff 4 | 434 | 434 | 434 | 434 | 434 | 434 | 434 | 434 | 434 |
| Sens 4 | 12% | 8% | 10% | 12% | 7% | 14% | 0% | 0% | 0% |
| Spec 4 | 91% | 90% | 92% | 91% | 90% | 92% | 91% | 90% | 92% |
| Cutoff 5 | 434 | 434 | 434 | 434 | 434 | 434 | 434 | 434 | 434 |
| Sens 5 | 12% | 8% | 10% | 12% | 7% | 14% | 0% | 0% | 0% |
| Spec 5 | 91% | 90% | 92% | 91% | 90% | 92% | 91% | 90% | 92% |
| Cutoff 6 | 434 | 434 | 434 | 434 | 434 | 434 | 434 | 434 | 434 |
| Sens 6 | 12% | 8% | 10% | 12% | 7% | 14% | 0% | 0% | 0% |
| Spec 6 | 91% | 90% | 92% | 91% | 90% | 92% | 91% | 90% | 92% |
| OR Quart 2 | 5.8 | 0.50 | 5.9 | 3.3 | 4.5 | 4.2 | 6.6 | >6.3 | 10 |
| p Value | 8.1E−5 | 0.57 | 1.4E−4 | 0.0094 | 0.0091 | 0.0014 | 0.085 | <0.090 | 0.029 |
| 95% CI of | 2.4 | 0.045 | 2.4 | 1.3 | 1.5 | 1.7 | 0.77 | >0.75 | 1.3 |
| OR Quart2 | 14 | 5.6 | 15 | 8.1 | 14 | 10 | 57 | na | 85 |
| OR Quart 3 | 9.2 | 9.1 | 8.8 | 9.7 | 1.5 | 5.5 | 28 | >6.3 | 23 |
| p Value | 6.4E−7 | 0.0037 | 2.6E−6 | 4.7E−7 | 0.52 | 1.4E−4 | 0.0014 | <0.090 | 0.0026 |
| 95% CI of | 3.8 | 2.1 | 3.5 | 4.0 | 0.42 | 2.3 | 3.7 | >0.75 | 3.0 |
| OR Quart3 | 22 | 41 | 22 | 23 | 5.6 | 13 | 220 | na | 180 |
| OR Quart 4 | 1.3 | 3.7 | 1.2 | 1.1 | 0.49 | 1.3 | 0 | >4.1 | 0 |
| p Value | 0.61 | 0.11 | 0.79 | 0.82 | 0.42 | 0.64 | na | <0.21 | na |
| 95% CI of | 0.48 | 0.75 | 0.40 | 0.41 | 0.088 | 0.47 | na | >0.46 | na |
| OR Quart4 | 3.5 | 18 | 3.4 | 3.1 | 2.7 | 3.4 | na | na | na |

TABLE 2
Comparison of marker levels in urine samples collected from Cohort 1 (patients that did not progress beyond RIFLE stage 0
or R) and in urine samples collected from subjects at 0, 24 hours, and 48 hours prior to reaching stage I or F in Cohort 2.
C-C motif chemokine 18

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 0.418 | 0.977 | 0.418 | 1.37 | 0.418 | 0.524 |
| Average | 2.19 | 3.40 | 2.19 | 5.34 | 2.19 | 6.72 |
| Stdev | 6.72 | 6.53 | 6.72 | 10.3 | 6.72 | 13.8 |
| p(t-test) | | 0.17 | | 3.2E−4 | | 1.1E−4 |
| Min | 3.13E−5 | 0.00308 | 3.13E−5 | 0.0139 | 3.13E−5 | 0.00215 |
| Max | 40.0 | 40.0 | 40.0 | 40.0 | 40.0 | 40.0 |
| n (Samp) | 927 | 62 | 927 | 70 | 927 | 39 |
| n (Patient) | 360 | 62 | 360 | 70 | 360 | 39 |
| sCr only | | | | | | |
| Median | 0.455 | 1.90 | 0.455 | 1.34 | 0.455 | 1.26 |
| Average | 2.54 | 8.19 | 2.54 | 6.52 | 2.54 | 5.50 |
| Stdev | 7.37 | 12.4 | 7.37 | 10.7 | 7.37 | 11.1 |
| p(t-test) | | 0.0035 | | 0.024 | | 0.10 |
| Min | 3.13E−5 | 0.192 | 3.13E−5 | 0.150 | 3.13E−5 | 0.0404 |
| Max | 40.0 | 40.0 | 40.0 | 40.0 | 40.0 | 37.8 |
| n (Samp) | 1230 | 15 | 1230 | 18 | 1230 | 17 |
| n (Patient) | 440 | 15 | 440 | 18 | 440 | 17 |
| UO only | | | | | | |
| Median | 0.456 | 0.951 | 0.456 | 1.71 | 0.456 | 0.692 |
| Average | 2.32 | 4.12 | 2.32 | 6.37 | 2.32 | 8.23 |
| Stdev | 6.87 | 8.22 | 6.87 | 11.6 | 6.87 | 14.6 |
| p(t-test) | | 0.060 | | 2.5E−5 | | 4.6E−6 |
| Min | 3.13E−5 | 0.00308 | 3.13E−5 | 0.0139 | 3.13E−5 | 0.00215 |
| Max | 40.0 | 40.0 | 40.0 | 40.0 | 40.0 | 40.0 |
| n (Samp) | 815 | 57 | 815 | 63 | 815 | 34 |
| n (Patient) | 282 | 57 | 282 | 63 | 282 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.65 | 0.79 | 0.63 | 0.67 | 0.70 | 0.68 | 0.58 | 0.66 | 0.60 |
| SE | 0.039 | 0.070 | 0.041 | 0.036 | 0.070 | 0.038 | 0.049 | 0.073 | 0.052 |
| p | 1.9E−4 | 2.6E−5 | 0.0015 | 2.2E−6 | 0.0046 | 4.5E−6 | 0.094 | 0.025 | 0.047 |
| nCohort 1 | 927 | 1230 | 815 | 927 | 1230 | 815 | 927 | 1230 | 815 |
| nCohort 2 | 62 | 15 | 57 | 70 | 18 | 63 | 39 | 17 | 34 |
| Cutoff 1 | 0.351 | 1.29 | 0.351 | 0.408 | 0.533 | 0.408 | 0.372 | 0.415 | 0.373 |
| Sens 1 | 71% | 73% | 70% | 70% | 72% | 71% | 72% | 71% | 71% |
| Spec 1 | 46% | 75% | 44% | 50% | 54% | 48% | 47% | 48% | 45% |
| Cutoff 2 | 0.231 | 0.985 | 0.231 | 0.282 | 0.340 | 0.282 | 0.131 | 0.388 | 0.131 |
| Sens 2 | 81% | 80% | 81% | 80% | 83% | 81% | 82% | 82% | 82% |
| Spec 2 | 33% | 69% | 31% | 41% | 43% | 38% | 21% | 47% | 19% |
| Cutoff 3 | 0.156 | 0.767 | 0.147 | 0.150 | 0.181 | 0.142 | 0.0423 | 0.225 | 0.0950 |
| Sens 3 | 90% | 93% | 91% | 90% | 94% | 90% | 92% | 94% | 91% |
| Spec 3 | 25% | 63% | 22% | 25% | 26% | 21% | 7% | 30% | 13% |
| Cutoff 4 | 0.900 | 1.02 | 0.982 | 0.900 | 1.02 | 0.982 | 0.900 | 1.02 | 0.982 |
| Sens 4 | 53% | 73% | 49% | 57% | 61% | 59% | 38% | 59% | 44% |
| Spec 4 | 70% | 70% | 70% | 70% | 70% | 70% | 70% | 70% | 70% |
| Cutoff 5 | 1.48 | 1.77 | 1.61 | 1.48 | 1.77 | 1.61 | 1.48 | 1.77 | 1.61 |
| Sens 5 | 42% | 53% | 42% | 49% | 44% | 51% | 33% | 35% | 38% |
| Spec 5 | 80% | 80% | 80% | 80% | 80% | 80% | 80% | 80% | 80% |
| Cutoff 6 | 3.30 | 3.86 | 3.53 | 3.30 | 3.86 | 3.53 | 3.30 | 3.86 | 3.53 |
| Sens 6 | 26% | 33% | 28% | 29% | 28% | 33% | 23% | 24% | 24% |
| Spec 6 | 90% | 90% | 90% | 90% | 90% | 90% | 90% | 90% | 90% |
| OR Quart 2 | 2.2 | >1.0 | 1.8 | 1.9 | 3.0 | 1.5 | 1.00 | 5.0 | 0.71 |
| p Value | 0.088 | <1.00 | 0.19 | 0.14 | 0.34 | 0.37 | 0.99 | 0.14 | 0.56 |
| 95% CI of | 0.89 | >0.062 | 0.74 | 0.80 | 0.31 | 0.61 | 0.37 | 0.59 | 0.22 |
| OR Quart2 | 5.5 | na | 4.4 | 4.6 | 29 | 3.8 | 2.7 | 43 | 2.3 |
| OR Quart 3 | 1.6 | >5.1 | 1.0 | 1.4 | 5.1 | 1.0 | 1.3 | 3.0 | 1.3 |
| p Value | 0.34 | <0.14 | 1.0 | 0.48 | 0.14 | 1.0 | 0.63 | 0.34 | 0.61 |
| 95% CI of | 0.61 | >0.59 | 0.37 | 0.55 | 0.59 | 0.37 | 0.49 | 0.31 | 0.47 |
| OR Quart3 | 4.2 | na | 2.7 | 3.5 | 44 | 2.7 | 3.3 | 29 | 3.6 |
| OR Quart 4 | 4.5 | >9.2 | 3.7 | 5.1 | 9.2 | 5.0 | 1.7 | 8.2 | 1.9 |
| p Value | 4.5E−4 | <0.035 | 0.0016 | 5.4E−5 | 0.035 | 7.1E−5 | 0.27 | 0.048 | 0.18 |
| 95% CI of | 1.9 | >1.2 | 1.6 | 2.3 | 1.2 | 2.3 | 0.67 | 1.0 | 0.74 |
| OR Quart4 | 11 | na | 8.4 | 11 | 73 | 11 | 4.1 | 66 | 4.9 |

C-C motif chemokine 24

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 11.1 | 26.1 | 11.1 | 27.2 | 11.1 | 13.3 |
| Average | 25.6 | 78.6 | 25.6 | 100 | 25.6 | 65.6 |
| Stdev | 60.0 | 180 | 60.0 | 331 | 60.0 | 197 |
| p(t-test) | | 4.5E−8 | | 1.1E−8 | | 5.4E−4 |
| Min | 0.0120 | 0.0123 | 0.0120 | 0.0347 | 0.0120 | 0.0123 |
| Max | 1090 | 1010 | 1090 | 2380 | 1090 | 1170 |
| n (Samp) | 928 | 62 | 928 | 70 | 928 | 39 |
| n (Patient) | 361 | 62 | 361 | 70 | 361 | 39 |
| sCr only | | | | | | |
| Median | 13.4 | 60.3 | 13.4 | 28.9 | 13.4 | 25.4 |
| Average | 37.8 | 104 | 37.8 | 60.4 | 37.8 | 46.1 |
| Stdev | 125 | 123 | 125 | 68.1 | 125 | 67.5 |
| p(t-test) | | 0.041 | | 0.44 | | 0.78 |
| Min | 0.0120 | 0.444 | 0.0120 | 2.39 | 0.0120 | 0.0196 |
| Max | 2380 | 373 | 2380 | 214 | 2380 | 249 |
| n (Samp) | 1232 | 15 | 1232 | 18 | 1232 | 17 |
| n (Patient) | 441 | 15 | 441 | 18 | 441 | 17 |
| UO only | | | | | | |
| Median | 11.3 | 26.8 | 11.3 | 27.4 | 11.3 | 15.7 |
| Average | 26.4 | 125 | 26.4 | 139 | 26.4 | 79.3 |
| Stdev | 63.0 | 403 | 63.0 | 416 | 63.0 | 210 |
| p(t-test) | | 1.9E−9 | | 1.9E−11 | | 5.4E−5 |
| Min | 0.0120 | 0.0123 | 0.0120 | 0.0347 | 0.0120 | 0.0123 |
| Max | 1090 | 2790 | 1090 | 2380 | 1090 | 1170 |
| n (Samp) | 817 | 57 | 817 | 63 | 817 | 34 |
| n (Patient) | 283 | 57 | 283 | 63 | 283 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.68 | 0.75 | 0.68 | 0.68 | 0.69 | 0.69 | 0.52 | 0.59 | 0.56 |
| SE | 0.039 | 0.073 | 0.040 | 0.036 | 0.070 | 0.038 | 0.048 | 0.073 | 0.052 |
| p | 5.1E−6 | 5.2E−4 | 5.7E−6 | 5.9E−7 | 0.0067 | 3.0E−7 | 0.61 | 0.20 | 0.22 |
| nCohort 1 | 928 | 1232 | 817 | 928 | 1232 | 817 | 928 | 1232 | 817 |
| nCohort 2 | 62 | 15 | 57 | 70 | 18 | 63 | 39 | 17 | 34 |
| Cutoff 1 | 10.4 | 25.9 | 11.3 | 14.5 | 15.6 | 14.5 | 4.24 | 14.9 | 4.59 |
| Sens 1 | 71% | 73% | 70% | 70% | 72% | 71% | 72% | 71% | 71% |
| Spec 1 | 48% | 69% | 50% | 57% | 55% | 58% | 32% | 54% | 33% |
| Cutoff 2 | 6.47 | 21.9 | 6.55 | 9.55 | 8.15 | 9.78 | 0.303 | 2.37 | 2.59 |
| Sens 2 | 81% | 80% | 81% | 80% | 83% | 81% | 82% | 82% | 82% |
| Spec 2 | 37% | 65% | 38% | 46% | 39% | 46% | 22% | 24% | 27% |
| Cutoff 3 | 2.85 | 2.85 | 1.36 | 4.66 | 3.22 | 5.39 | 0.0277 | 0.0277 | 0.0562 |
| Sens 3 | 90% | 93% | 91% | 90% | 94% | 90% | 92% | 94% | 91% |
| Spec 3 | 28% | 25% | 24% | 33% | 26% | 35% | 8% | 7% | 19% |
| Cutoff 4 | 22.3 | 26.6 | 21.8 | 22.3 | 26.6 | 21.8 | 22.3 | 26.6 | 21.8 |
| Sens 4 | 58% | 67% | 60% | 57% | 61% | 59% | 36% | 41% | 44% |
| Spec 4 | 70% | 70% | 70% | 70% | 70% | 70% | 70% | 70% | 70% |
| Cutoff 5 | 34.1 | 40.7 | 33.4 | 34.1 | 40.7 | 33.4 | 34.1 | 40.7 | 33.4 |
| Sens 5 | 42% | 67% | 44% | 39% | 39% | 41% | 21% | 24% | 26% |
| Spec 5 | 80% | 80% | 80% | 80% | 80% | 80% | 80% | 80% | 80% |
| Cutoff 6 | 57.3 | 69.9 | 62.1 | 57.3 | 69.9 | 62.1 | 57.3 | 69.9 | 62.1 |
| Sens 6 | 27% | 33% | 28% | 20% | 28% | 21% | 15% | 18% | 21% |
| Spec 6 | 90% | 90% | 90% | 90% | 90% | 90% | 90% | 90% | 90% |
| OR Quart 2 | 2.4 | 0.50 | 2.0 | 5.2 | 3.0 | 4.2 | 1.3 | 0.25 | 1.5 |
| p Value | 0.077 | 0.57 | 0.16 | 0.0096 | 0.34 | 0.029 | 0.64 | 0.21 | 0.44 |
| 95% CI of | 0.91 | 0.045 | 0.75 | 1.5 | 0.31 | 1.2 | 0.49 | 0.028 | 0.53 |
| OR Quart2 | 6.4 | 5.5 | 5.6 | 18 | 29 | 15 | 3.2 | 2.2 | 4.3 |
| OR Quart 3 | 2.4 | 1.00 | 2.1 | 6.8 | 6.1 | 6.1 | 1.5 | 2.0 | 1.5 |
| p Value | 0.076 | 1.00 | 0.16 | 0.0023 | 0.095 | 0.0045 | 0.37 | 0.25 | 0.44 |
| 95% CI of | 0.91 | 0.14 | 0.76 | 2.0 | 0.73 | 1.7 | 0.61 | 0.60 | 0.53 |
| OR Quart3 | 6.4 | 7.1 | 5.6 | 23 | 51 | 21 | 3.8 | 6.8 | 4.3 |
| OR Quart 4 | 5.1 | 5.1 | 5.0 | 12 | 8.2 | 12 | 1.1 | 1.00 | 1.7 |
| p Value | 3.8E−4 | 0.036 | 5.2E−4 | 3.5E−5 | 0.048 | 5.4E−5 | 0.81 | 1.00 | 0.32 |
| 95% CI of | 2.1 | 1.1 | 2.0 | 3.8 | 1.0 | 3.6 | 0.43 | 0.25 | 0.60 |
| OR Quart4 | 13 | 24 | 12 | 41 | 66 | 39 | 3.0 | 4.0 | 4.7 |

C-C motif chemokine 8

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 1.21 | 5.60 | 1.21 | 6.10 | 1.21 | 1.51 |
| Average | 9.02 | 18.6 | 9.02 | 19.2 | 9.02 | 18.6 |
| Stdev | 25.8 | 33.5 | 25.8 | 42.6 | 25.8 | 41.8 |
| p(t-test) | | 0.0056 | | 0.0027 | | 0.028 |
| Min | 0.0250 | 0.0775 | 0.0250 | 0.0250 | 0.0250 | 0.0250 |
| Max | 473 | 162 | 473 | 294 | 473 | 214 |
| n (Samp) | 928 | 62 | 928 | 70 | 928 | 39 |
| n (Patient) | 361 | 62 | 361 | 70 | 361 | 39 |
| sCr only | | | | | | |
| Median | 1.36 | 7.59 | 1.36 | 8.78 | 1.36 | 2.77 |
| Average | 11.2 | 23.8 | 11.2 | 40.3 | 11.2 | 11.6 |
| Stdev | 32.4 | 36.8 | 32.4 | 75.9 | 32.4 | 16.3 |
| p(t-test) | | 0.14 | | 2.5E−4 | | 0.96 |
| Min | 0.0250 | 0.127 | 0.0250 | 0.0569 | 0.0250 | 0.0250 |
| Max | 492 | 116 | 492 | 279 | 492 | 46.5 |
| n (Samp) | 1232 | 15 | 1232 | 18 | 1232 | 17 |
| n (Patient) | 441 | 15 | 441 | 18 | 441 | 17 |
| UO only | | | | | | |
| Median | 1.21 | 5.77 | 1.21 | 6.84 | 1.21 | 2.14 |
| Average | 9.52 | 22.6 | 9.52 | 22.9 | 9.52 | 20.3 |
| Stdev | 27.3 | 39.9 | 27.3 | 45.9 | 27.3 | 44.5 |
| p(t-test) | | 7.8E−4 | | 4.5E−4 | | 0.029 |
| Min | 0.0250 | 0.0775 | 0.0250 | 0.0250 | 0.0250 | 0.0250 |
| Max | 473 | 176 | 473 | 294 | 473 | 214 |
| n (Samp) | 817 | 57 | 817 | 63 | 817 | 34 |
| n (Patient) | 283 | 57 | 283 | 63 | 283 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.67 | 0.63 | 0.69 | 0.65 | 0.67 | 0.66 | 0.56 | 0.55 | 0.57 |
| SE | 0.039 | 0.078 | 0.040 | 0.037 | 0.071 | 0.039 | 0.049 | 0.072 | 0.052 |
| p | 8.0E−6 | 0.093 | 2.4E−6 | 4.7E−5 | 0.016 | 2.2E−5 | 0.18 | 0.49 | 0.16 |
| nCohort 1 | 928 | 1232 | 817 | 928 | 1232 | 817 | 928 | 1232 | 817 |
| nCohort 2 | 62 | 15 | 57 | 70 | 18 | 63 | 39 | 17 | 34 |
| Cutoff 1 | 1.55 | 0.240 | 2.74 | 1.21 | 3.16 | 1.55 | 0.438 | 0.802 | 0.453 |
| Sens 1 | 71% | 73% | 70% | 71% | 72% | 71% | 72% | 71% | 71% |
| Spec 1 | 54% | 33% | 59% | 51% | 59% | 54% | 41% | 43% | 41% |
| Cutoff 2 | 0.802 | 0.239 | 0.802 | 0.240 | 0.239 | 0.453 | 0.154 | 0.127 | 0.240 |
| Sens 2 | 82% | 80% | 86% | 81% | 89% | 81% | 85% | 82% | 85% |
| Spec 2 | 45% | 31% | 46% | 36% | 31% | 41% | 29% | 21% | 35% |
| Cutoff 3 | 0.154 | 0.127 | 0.240 | 0.154 | 0.154 | 0.127 | 0.0775 | 0.0705 | 0.127 |
| Sens 3 | 92% | 93% | 91% | 90% | 94% | 90% | 92% | 94% | 91% |
| Spec 3 | 29% | 21% | 35% | 29% | 27% | 21% | 16% | 11% | 21% |
| Cutoff 4 | 5.57 | 6.87 | 5.57 | 5.57 | 6.87 | 5.57 | 5.57 | 6.87 | 5.57 |
| Sens 4 | 50% | 53% | 51% | 53% | 61% | 56% | 33% | 35% | 35% |
| Spec 4 | 70% | 70% | 70% | 70% | 70% | 70% | 70% | 70% | 70% |
| Cutoff 5 | 10.6 | 12.1 | 11.1 | 10.6 | 12.1 | 11.1 | 10.6 | 12.1 | 11.1 |
| Sens 5 | 39% | 33% | 40% | 30% | 44% | 33% | 31% | 35% | 24% |
| Spec 5 | 80% | 80% | 80% | 80% | 80% | 80% | 80% | 80% | 80% |
| Cutoff 6 | 21.9 | 26.4 | 22.0 | 21.9 | 26.4 | 22.0 | 21.9 | 26.4 | 22.0 |
| Sens 6 | 24% | 27% | 28% | 20% | 22% | 25% | 18% | 18% | 18% |
| Spec 6 | 90% | 90% | 90% | 90% | 90% | 90% | 90% | 90% | 90% |
| OR Quart 2 | 5.2 | 1.5 | 2.8 | 2.4 | 4.0 | 1.4 | 2.7 | 1.0 | 3.1 |
| p Value | 0.0096 | 0.66 | 0.079 | 0.077 | 0.21 | 0.46 | 0.065 | 1.0 | 0.053 |
| 95% CI of | 1.5 | 0.25 | 0.89 | 0.91 | 0.45 | 0.54 | 0.94 | 0.25 | 0.98 |
| OR Quart2 | 18 | 9.0 | 9.0 | 6.4 | 36 | 3.9 | 7.6 | 4.0 | 9.8 |
| OR Quart 3 | 6.4 | 1.5 | 4.5 | 4.1 | 4.0 | 3.0 | 1.8 | 0.75 | 2.0 |
| p Value | 0.0032 | 0.66 | 0.0075 | 0.0025 | 0.21 | 0.013 | 0.29 | 0.70 | 0.25 |
| 95% CI of | 1.9 | 0.25 | 1.5 | 1.6 | 0.45 | 1.3 | 0.60 | 0.17 | 0.60 |
| OR Quart3 | 22 | 9.0 | 14 | 10 | 36 | 7.4 | 5.5 | 3.4 | 6.8 |
| OR Quart 4 | 9.5 | 3.5 | 6.9 | 4.9 | 9.2 | 4.1 | 2.5 | 1.5 | 2.6 |
| p Value | 2.6E−4 | 0.12 | 4.2E−4 | 5.6E−4 | 0.036 | 0.0013 | 0.095 | 0.53 | 0.12 |
| 95% CI of | 2.8 | 0.73 | 2.4 | 2.0 | 1.2 | 1.7 | 0.85 | 0.42 | 0.79 |
| OR Quart4 | 32 | 17 | 20 | 12 | 73 | 9.6 | 7.1 | 5.4 | 8.3 |

Cathepsin D

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 72900 | 109000 | 72900 | 93800 | 72900 | 87500 |
| Average | 77100 | 113000 | 77100 | 103000 | 77100 | 90300 |
| Stdev | 39300 | 46500 | 39300 | 43200 | 39300 | 45100 |
| p(t-test) | | 8.8E−12 | | 1.9E−7 | | 0.042 |
| Min | 656 | 3710 | 656 | 22600 | 656 | 4320 |
| Max | 200000 | 200000 | 200000 | 200000 | 200000 | 200000 |
| n (Samp) | 927 | 62 | 927 | 70 | 927 | 39 |
| n (Patient) | 360 | 62 | 360 | 70 | 360 | 39 |
| sCr only | | | | | | |
| Median | 76100 | 141000 | 76100 | 98000 | 76100 | 111000 |
| Average | 82300 | 128000 | 82300 | 112000 | 82300 | 97600 |
| Stdev | 42300 | 39000 | 42300 | 43000 | 42300 | 43200 |
| p(t-test) | | 3.2E−5 | | 0.0034 | | 0.14 |
| Min | 656 | 70300 | 656 | 52100 | 656 | 22100 |
| Max | 200000 | 193000 | 200000 | 200000 | 200000 | 200000 |
| n (Samp) | 1230 | 15 | 1230 | 18 | 1230 | 17 |
| n (Patient) | 440 | 15 | 440 | 18 | 440 | 17 |
| UO only | | | | | | |
| Median | 74300 | 111000 | 74300 | 97500 | 74300 | 94700 |
| Average | 79900 | 114000 | 79900 | 105000 | 79900 | 97200 |
| Stdev | 38900 | 47800 | 38900 | 44600 | 38900 | 44600 |
| p(t-test) | | 4.5E−10 | | 9.0E−7 | | 0.012 |
| Min | 2520 | 3710 | 2520 | 22600 | 2520 | 4320 |
| Max | 200000 | 200000 | 200000 | 200000 | 200000 | 200000 |
| n (Samp) | 815 | 57 | 815 | 63 | 815 | 34 |
| n (Patient) | 282 | 57 | 282 | 63 | 282 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.73 | 0.79 | 0.72 | 0.67 | 0.69 | 0.67 | 0.59 | 0.61 | 0.63 |
| SE | 0.037 | 0.070 | 0.039 | 0.036 | 0.070 | 0.038 | 0.049 | 0.073 | 0.052 |
| p | 1.1E−9 | 3.2E−5 | 4.4E−8 | 2.2E−6 | 0.0060 | 1.4E−5 | 0.067 | 0.13 | 0.015 |
| nCohort 1 | 927 | 1230 | 815 | 927 | 1230 | 815 | 927 | 1230 | 815 |
| nCohort 2 | 62 | 15 | 57 | 70 | 18 | 63 | 39 | 17 | 34 |
| Cutoff 1 | 83000 | 100000 | 84700 | 78100 | 84700 | 77500 | 65500 | 70400 | 73300 |
| Sens 1 | 71% | 73% | 70% | 70% | 72% | 71% | 72% | 71% | 71% |
| Spec 1 | 62% | 70% | 60% | 57% | 58% | 53% | 43% | 44% | 49% |
| Cutoff 2 | 73800 | 94300 | 76300 | 68200 | 79000 | 68200 | 55600 | 55600 | 65500 |
| Sens 2 | 81% | 80% | 81% | 80% | 83% | 81% | 82% | 82% | 82% |
| Spec 2 | 52% | 66% | 53% | 45% | 53% | 43% | 31% | 27% | 40% |
| Cutoff 3 | 61400 | 73800 | 59600 | 54900 | 53700 | 55200 | 38800 | 40900 | 54900 |
| Sens 3 | 90% | 93% | 91% | 90% | 94% | 90% | 92% | 94% | 91% |
| Spec 3 | 37% | 47% | 33% | 30% | 25% | 28% | 15% | 16% | 27% |
| Cutoff 4 | 92900 | 101000 | 96900 | 92900 | 101000 | 96900 | 92900 | 101000 | 96900 |
| Sens 4 | 61% | 67% | 61% | 51% | 50% | 51% | 46% | 59% | 47% |
| Spec 4 | 70% | 70% | 70% | 70% | 70% | 70% | 70% | 70% | 70% |
| Cutoff 5 | 111000 | 117000 | 112000 | 111000 | 117000 | 112000 | 111000 | 117000 | 112000 |
| Sens 5 | 48% | 53% | 49% | 39% | 44% | 40% | 26% | 35% | 26% |
| Spec 5 | 80% | 80% | 80% | 80% | 80% | 80% | 80% | 80% | 80% |
| Cutoff 6 | 130000 | 140000 | 133000 | 130000 | 140000 | 133000 | 130000 | 140000 | 133000 |
| Sens 6 | 37% | 53% | 32% | 23% | 28% | 24% | 18% | 6% | 21% |
| Spec 6 | 90% | 90% | 90% | 90% | 90% | 90% | 90% | 90% | 90% |
| OR Quart 2 | 2.3 | >2.0 | 1.2 | 2.9 | 2.0 | 2.7 | 2.0 | 2.0 | 2.7 |
| p Value | 0.17 | <0.57 | 0.76 | 0.044 | 0.57 | 0.065 | 0.20 | 0.42 | 0.14 |
| 95% CI of | 0.70 | >0.18 | 0.36 | 1.0 | 0.18 | 0.94 | 0.68 | 0.36 | 0.71 |
| OR Quart2 | 7.6 | na | 4.0 | 8.2 | 22 | 7.7 | 6.0 | 11 | 10 |
| OR Quart 3 | 4.2 | >4.1 | 3.1 | 4.7 | 7.1 | 3.8 | 2.0 | 1.00 | 3.1 |
| p Value | 0.011 | <0.21 | 0.029 | 0.0020 | 0.067 | 0.0091 | 0.20 | 1.00 | 0.094 |
| 95% CI of | 1.4 | >0.45 | 1.1 | 1.8 | 0.87 | 1.4 | 0.69 | 0.14 | 0.82 |
| OR Quart3 | 13 | na | 8.8 | 13 | 58 | 11 | 6.1 | 7.1 | 12 |
| OR Quart 4 | 9.3 | >9.2 | 7.1 | 6.4 | 8.2 | 6.0 | 2.9 | 4.6 | 4.9 |
| p Value | 3.3E−5 | <0.035 | 7.2E−5 | 1.7E−4 | 0.048 | 3.2E−4 | 0.044 | 0.053 | 0.014 |
| 95% CI of | 3.3 | >1.2 | 2.7 | 2.4 | 1.0 | 2.3 | 1.0 | 0.98 | 1.4 |
| OR Quart4 | 27 | na | 19 | 17 | 66 | 16 | 8.2 | 21 | 17 |

C-X-C motif chemokine 13

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 0.0222 | 1.42 | 0.0222 | 1.46 | 0.0222 | 0.232 |
| Average | 5.79 | 13.4 | 5.79 | 16.4 | 5.79 | 17.2 |
| Stdev | 38.4 | 34.8 | 38.4 | 33.5 | 38.4 | 71.5 |
| p(t-test) | | 0.13 | | 0.025 | | 0.083 |
| Min | 0.00269 | 0.00471 | 0.00269 | 0.00269 | 0.00269 | 0.00269 |
| Max | 832 | 210 | 832 | 131 | 832 | 440 |
| n (Samp) | 928 | 62 | 928 | 70 | 928 | 39 |
| n (Patient) | 361 | 62 | 361 | 70 | 361 | 39 |
| sCr only | | | | | | |
| Median | 0.115 | 3.89 | 0.115 | 3.31 | 0.115 | 1.39 |
| Average | 8.27 | 22.4 | 8.27 | 20.2 | 8.27 | 9.64 |
| Stdev | 41.2 | 45.9 | 41.2 | 50.6 | 41.2 | 20.1 |
| p(t-test) | | 0.19 | | 0.22 | | 0.89 |
| Min | 0.00269 | 0.00627 | 0.00269 | 0.00822 | 0.00269 | 0.00269 |
| Max | 832 | 171 | 832 | 210 | 832 | 79.0 |
| n (Samp) | 1232 | 15 | 1232 | 18 | 1232 | 17 |
| n (Patient) | 441 | 15 | 441 | 18 | 441 | 17 |
| UO only | | | | | | |
| Median | 0.0222 | 1.79 | 0.0222 | 1.86 | 0.0222 | 1.09 |
| Average | 6.06 | 48.6 | 6.06 | 48.7 | 6.06 | 20.3 |
| Stdev | 40.6 | 257 | 40.6 | 234 | 40.6 | 76.3 |
| p(t-test) | | 4.8E−5 | | 1.0E−5 | | 0.057 |
| Min | 0.00269 | 0.00471 | 0.00269 | 0.00269 | 0.00269 | 0.00269 |
| Max | 832 | 1930 | 832 | 1850 | 832 | 440 |
| n (Samp) | 817 | 57 | 817 | 63 | 817 | 34 |
| n (Patient) | 283 | 57 | 283 | 63 | 283 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.67 | 0.68 | 0.69 | 0.67 | 0.68 | 0.69 | 0.56 | 0.56 | 0.62 |
| SE | 0.039 | 0.077 | 0.040 | 0.036 | 0.070 | 0.038 | 0.048 | 0.073 | 0.052 |
| p | 8.2E−6 | 0.020 | 1.1E−6 | 2.1E−6 | 0.012 | 3.2E−7 | 0.23 | 0.43 | 0.020 |
| nCohort 1 | 928 | 1232 | 817 | 928 | 1232 | 817 | 928 | 1232 | 817 |
| nCohort 2 | 62 | 15 | 57 | 70 | 18 | 63 | 39 | 17 | 34 |
| Cutoff 1 | 0.352 | 1.02 | 0.362 | 0.335 | 0.605 | 0.392 | 0.0109 | 0.0130 | 0.0174 |
| Sens 1 | 71% | 73% | 72% | 70% | 72% | 71% | 74% | 71% | 71% |
| Spec 1 | 58% | 64% | 58% | 58% | 58% | 59% | 29% | 33% | 47% |
| Cutoff 2 | 0.0126 | 0.0109 | 0.0193 | 0.0130 | 0.0109 | 0.0140 | 0.00930 | 0.00564 | 0.0109 |
| Sens 2 | 81% | 80% | 81% | 80% | 83% | 81% | 82% | 82% | 82% |
| Spec 2 | 34% | 25% | 48% | 36% | 25% | 37% | 28% | 13% | 27% |
| Cutoff 3 | 0.00930 | 0.00756 | 0.00930 | 0.00834 | 0.00821 | 0.0109 | 0.00564 | 0.00269 | 0.00930 |
| Sens 3 | 90% | 93% | 95% | 90% | 100% | 90% | 92% | 94% | 91% |
| Spec 3 | 28% | 15% | 26% | 25% | 19% | 27% | 15% | 2% | 26% |
| Cutoff 4 | 1.13 | 1.62 | 1.13 | 1.13 | 1.62 | 1.13 | 1.13 | 1.62 | 1.13 |
| Sens 4 | 56% | 67% | 60% | 56% | 67% | 59% | 44% | 47% | 50% |
| Spec 4 | 70% | 70% | 70% | 70% | 70% | 70% | 70% | 70% | 70% |
| Cutoff 5 | 2.67 | 3.52 | 2.66 | 2.67 | 3.52 | 2.66 | 2.67 | 3.52 | 2.66 |
| Sens 5 | 39% | 53% | 44% | 40% | 50% | 43% | 28% | 35% | 32% |
| Spec 5 | 80% | 80% | 80% | 80% | 80% | 80% | 80% | 80% | 80% |
| Cutoff 6 | 8.75 | 11.3 | 8.56 | 8.75 | 11.3 | 8.56 | 8.75 | 11.3 | 8.56 |
| Sens 6 | 26% | 33% | 28% | 26% | 22% | 30% | 15% | 18% | 21% |
| Spec 6 | 90% | 90% | 90% | 90% | 90% | 90% | 90% | 90% | 90% |
| OR Quart 2 | 2.7 | 0.33 | 2.0 | 1.9 | 0.66 | 1.5 | 1.4 | 0.20 | 3.1 |
| p Value | 0.065 | 0.34 | 0.20 | 0.18 | 0.65 | 0.43 | 0.47 | 0.14 | 0.096 |
| 95% CI of | 0.94 | 0.034 | 0.69 | 0.74 | 0.11 | 0.53 | 0.54 | 0.023 | 0.82 |
| OR Quart2 | 7.6 | 3.2 | 6.1 | 4.8 | 4.0 | 4.3 | 3.9 | 1.7 | 12 |
| OR Quart 3 | 3.4 | 0.66 | 3.1 | 2.9 | 0.66 | 3.6 | 1.1 | 0.60 | 2.7 |
| p Value | 0.020 | 0.65 | 0.029 | 0.020 | 0.66 | 0.0075 | 0.80 | 0.48 | 0.14 |
| 95% CI of | 1.2 | 0.11 | 1.1 | 1.2 | 0.11 | 1.4 | 0.41 | 0.14 | 0.71 |
| OR Quart3 | 9.3 | 4.0 | 8.8 | 6.9 | 4.0 | 9.1 | 3.2 | 2.5 | 10 |
| OR Quart 4 | 6.2 | 3.0 | 6.0 | 4.9 | 3.8 | 5.2 | 2.1 | 1.6 | 4.9 |
| p Value | 2.4E−4 | 0.097 | 3.2E−4 | 2.1E−4 | 0.044 | 3.4E−4 | 0.13 | 0.41 | 0.014 |
| 95% CI of | 2.3 | 0.82 | 2.3 | 2.1 | 1.0 | 2.1 | 0.81 | 0.52 | 1.4 |
| OR Quart4 | 16 | 11 | 16 | 11 | 14 | 13 | 5.2 | 5.0 | 17 |

Insulin-like growth factor-binding protein 3

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 355 | 654 | 355 | 566 | 355 | 559 |
| Average | 611 | 1050 | 611 | 1210 | 611 | 688 |
| Stdev | 946 | 1310 | 946 | 2170 | 946 | 574 |
| p(t-test) | | 3.3E−4 | | 2.2E−6 | | 0.59 |
| Min | 0.0478 | 17.3 | 0.0478 | 32.5 | 0.0478 | 0.311 |
| Max | 12500 | 7720 | 12500 | 12500 | 12500 | 2100 |
| n (Samp) | 1005 | 70 | 1005 | 80 | 1005 | 46 |
| n (Patient) | 386 | 70 | 386 | 80 | 386 | 46 |
| sCr only | | | | | | |
| Median | 408 | 542 | 408 | 618 | 408 | 540 |
| Average | 715 | 1100 | 715 | 1280 | 715 | 797 |
| Stdev | 1130 | 1190 | 1130 | 1880 | 1130 | 745 |
| p(t-test) | | 0.17 | | 0.024 | | 0.74 |
| Min | 0.0478 | 38.8 | 0.0478 | 44.3 | 0.0478 | 44.1 |
| Max | 12500 | 4040 | 12500 | 8160 | 12500 | 2990 |
| n (Samp) | 1341 | 16 | 1341 | 21 | 1341 | 20 |
| n (Patient) | 472 | 16 | 472 | 21 | 472 | 20 |
| UO only | | | | | | |
| Median | 367 | 713 | 367 | 646 | 367 | 701 |
| Average | 630 | 1320 | 630 | 1340 | 630 | 812 |
| Stdev | 1030 | 1980 | 1030 | 2260 | 1030 | 675 |
| p(t-test) | | 1.7E−6 | | 6.5E−7 | | 0.27 |
| Min | 0.0478 | 17.3 | 0.0478 | 32.5 | 0.0478 | 0.311 |
| Max | 12500 | 12500 | 12500 | 12500 | 12500 | 2870 |
| n (Samp) | 897 | 65 | 897 | 73 | 897 | 40 |
| n (Patient) | 310 | 65 | 310 | 73 | 310 | 40 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.65 | 0.61 | 0.67 | 0.62 | 0.62 | 0.64 | 0.57 | 0.58 | 0.61 |
| SE | 0.037 | 0.075 | 0.038 | 0.035 | 0.066 | 0.036 | 0.045 | 0.067 | 0.048 |
| p | 3.7E−5 | 0.13 | 4.9E−6 | 3.5E−4 | 0.080 | 6.4E−5 | 0.097 | 0.22 | 0.023 |
| nCohort 1 | 1005 | 1341 | 897 | 1005 | 1341 | 897 | 1005 | 1341 | 897 |
| nCohort 2 | 70 | 16 | 65 | 80 | 21 | 73 | 46 | 20 | 40 |
| Cutoff 1 | 408 | 212 | 411 | 313 | 287 | 319 | 201 | 294 | 271 |
| Sens 1 | 70% | 75% | 71% | 70% | 71% | 71% | 72% | 70% | 70% |
| Spec 1 | 54% | 33% | 53% | 46% | 40% | 45% | 35% | 41% | 40% |
| Cutoff 2 | 259 | 210 | 262 | 205 | 259 | 245 | 138 | 249 | 191 |
| Sens 2 | 80% | 81% | 80% | 80% | 81% | 81% | 80% | 80% | 80% |
| Spec 2 | 42% | 33% | 40% | 35% | 38% | 38% | 27% | 37% | 31% |
| Cutoff 3 | 118 | 163 | 118 | 114 | 163 | 118 | 43.4 | 139 | 52.6 |
| Sens 3 | 90% | 94% | 91% | 90% | 90% | 90% | 91% | 90% | 90% |
| Spec 3 | 24% | 26% | 21% | 23% | 26% | 21% | 8% | 24% | 9% |
| Cutoff 4 | 651 | 746 | 645 | 651 | 746 | 645 | 651 | 746 | 645 |
| Sens 4 | 51% | 44% | 57% | 45% | 43% | 51% | 46% | 45% | 50% |
| Spec 4 | 70% | 70% | 70% | 70% | 70% | 70% | 70% | 70% | 70% |
| Cutoff 5 | 922 | 1040 | 926 | 922 | 1040 | 926 | 922 | 1040 | 926 |
| Sens 5 | 33% | 38% | 37% | 35% | 43% | 38% | 33% | 30% | 40% |
| Spec 5 | 80% | 80% | 80% | 80% | 80% | 80% | 80% | 80% | 80% |
| Cutoff 6 | 1350 | 1540 | 1330 | 1350 | 1540 | 1330 | 1350 | 1540 | 1330 |
| Sens 6 | 23% | 19% | 26% | 22% | 19% | 27% | 15% | 10% | 22% |
| Spec 6 | 90% | 90% | 90% | 90% | 90% | 90% | 90% | 90% | 90% |
| OR Quart 2 | 1.4 | 4.0 | 1.00 | 1.8 | 2.5 | 1.6 | 1.00 | 2.0 | 1.1 |
| p Value | 0.49 | 0.21 | 0.99 | 0.14 | 0.27 | 0.23 | 0.99 | 0.32 | 0.79 |
| 95% CI of | 0.55 | 0.45 | 0.37 | 0.83 | 0.48 | 0.73 | 0.39 | 0.50 | 0.41 |
| OR Quart2 | 3.5 | 36 | 2.7 | 3.8 | 13 | 3.7 | 2.6 | 8.1 | 3.2 |
| OR Quart 3 | 2.9 | 4.0 | 2.5 | 1.8 | 2.5 | 1.8 | 1.1 | 1.3 | 0.85 |
| p Value | 0.012 | 0.21 | 0.034 | 0.14 | 0.27 | 0.17 | 0.82 | 0.70 | 0.78 |
| 95% CI of | 1.3 | 0.45 | 1.1 | 0.83 | 0.49 | 0.79 | 0.44 | 0.30 | 0.28 |
| OR Quart3 | 6.6 | 36 | 5.8 | 3.8 | 13 | 3.9 | 2.8 | 6.0 | 2.6 |
| OR Quart 4 | 3.9 | 7.1 | 4.1 | 3.0 | 4.6 | 3.3 | 2.1 | 2.4 | 2.9 |
| p Value | 8.3E−4 | 0.067 | 5.3E−4 | 0.0021 | 0.053 | 0.0017 | 0.083 | 0.22 | 0.020 |
| 95% CI of | 1.8 | 0.87 | 1.8 | 1.5 | 0.98 | 1.6 | 0.91 | 0.60 | 1.2 |
| OR Quart4 | 8.8 | 58 | 9.2 | 6.2 | 21 | 6.8 | 4.7 | 9.2 | 6.9 |

Immunoglogulin G1

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 3060 | 5910 | 3060 | 7970 | 3060 | 4550 |
| Average | 5430 | 8350 | 5430 | 10300 | 5430 | 6440 |
| Stdev | 6740 | 7390 | 6740 | 11400 | 6740 | 6380 |
| p(t-test) | | 0.0011 | | 6.3E−8 | | 0.36 |
| Min | 59.7 | 447 | 59.7 | 634 | 59.7 | 55.9 |
| Max | 80000 | 35700 | 80000 | 80000 | 80000 | 25300 |
| n (Samp) | 922 | 62 | 922 | 70 | 922 | 39 |
| n (Patient) | 358 | 62 | 358 | 70 | 358 | 39 |
| sCr only | | | | | | |
| Median | 3410 | 5220 | 3410 | 10900 | 3410 | 9840 |
| Average | 6040 | 9080 | 6040 | 10000 | 6040 | 9430 |
| Stdev | 7380 | 7850 | 7380 | 8360 | 7380 | 7740 |
| p(t-test) | | 0.11 | | 0.023 | | 0.060 |
| Min | 3.36 | 504 | 3.36 | 658 | 3.36 | 177 |
| Max | 80000 | 24700 | 80000 | 26100 | 80000 | 23800 |
| n (Samp) | 1225 | 15 | 1225 | 18 | 1225 | 17 |
| n (Patient) | 438 | 15 | 438 | 18 | 438 | 17 |
| UO only | | | | | | |
| Median | 3060 | 6850 | 3060 | 8300 | 3060 | 5080 |
| Average | 5550 | 9370 | 5550 | 11400 | 5550 | 7220 |
| Stdev | 6860 | 7890 | 6860 | 12000 | 6860 | 6330 |
| p(t-test) | | 6.2E−5 | | 2.1E−9 | | 0.16 |
| Min | 33.0 | 447 | 33.0 | 634 | 33.0 | 55.9 |
| Max | 80000 | 35700 | 80000 | 80000 | 80000 | 25300 |
| n (Samp) | 810 | 57 | 810 | 63 | 810 | 34 |
| n (Patient) | 280 | 57 | 280 | 63 | 280 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.66 | 0.64 | 0.69 | 0.67 | 0.61 | 0.71 | 0.55 | 0.60 | 0.61 |
| SE | 0.039 | 0.078 | 0.040 | 0.036 | 0.071 | 0.038 | 0.048 | 0.073 | 0.052 |
| p | 3.4E−5 | 0.068 | 3.2E−6 | 2.5E−6 | 0.11 | 2.0E−8 | 0.35 | 0.17 | 0.031 |
| nCohort 1 | 922 | 1225 | 810 | 922 | 1225 | 810 | 922 | 1225 | 810 |
| nCohort 2 | 62 | 15 | 57 | 70 | 18 | 63 | 39 | 17 | 34 |
| Cutoff 1 | 3550 | 3550 | 4050 | 4350 | 2320 | 4540 | 1740 | 1740 | 2930 |
| Sens 1 | 71% | 73% | 70% | 70% | 72% | 71% | 72% | 71% | 71% |
| Spec 1 | 57% | 52% | 61% | 64% | 34% | 65% | 27% | 25% | 48% |
| Cutoff 2 | 2720 | 2920 | 2720 | 2010 | 1090 | 2630 | 1390 | 831 | 1740 |
| Sens 2 | 81% | 80% | 81% | 80% | 83% | 81% | 82% | 82% | 82% |
| Spec 2 | 45% | 43% | 46% | 33% | 13% | 44% | 20% | 9% | 28% |
| Cutoff 3 | 1130 | 933 | 1880 | 1190 | 673 | 1600 | 541 | 541 | 1390 |
| Sens 3 | 90% | 93% | 91% | 90% | 94% | 90% | 92% | 94% | 91% |
| Spec 3 | 15% | 11% | 30% | 16% | 6% | 24% | 5% | 5% | 21% |
| Cutoff 4 | 5430 | 6270 | 5590 | 5430 | 6270 | 5590 | 5430 | 6270 | 5590 |
| Sens 4 | 52% | 47% | 58% | 60% | 56% | 62% | 44% | 59% | 47% |
| Spec 4 | 70% | 70% | 70% | 70% | 70% | 70% | 70% | 70% | 70% |
| Cutoff 5 | 8100 | 8950 | 8470 | 8100 | 8950 | 8470 | 8100 | 8950 | 8470 |
| Sens 5 | 42% | 33% | 44% | 49% | 56% | 49% | 31% | 53% | 32% |
| Spec 5 | 80% | 80% | 80% | 80% | 80% | 80% | 80% | 80% | 80% |
| Cutoff 6 | 13100 | 14200 | 13400 | 13100 | 14200 | 13400 | 13100 | 14200 | 13400 |
| Sens 6 | 18% | 27% | 23% | 31% | 44% | 32% | 18% | 29% | 21% |
| Spec 6 | 90% | 90% | 90% | 90% | 90% | 90% | 90% | 90% | 90% |
| OR Quart 2 | 1.4 | 1.0 | 1.8 | 0.57 | 1.00 | 0.62 | 0.69 | 0.20 | 0.83 |
| p Value | 0.46 | 1.0 | 0.29 | 0.25 | 1.00 | 0.40 | 0.46 | 0.14 | 0.76 |
| 95% CI of | 0.54 | 0.14 | 0.60 | 0.22 | 0.25 | 0.20 | 0.26 | 0.023 | 0.25 |
| OR Quart2 | 3.9 | 7.1 | 5.5 | 1.5 | 4.0 | 1.9 | 1.8 | 1.7 | 2.8 |
| OR Quart 3 | 2.7 | 2.5 | 3.1 | 1.2 | 0 | 1.9 | 0.90 | 0.20 | 1.9 |
| p Value | 0.029 | 0.27 | 0.030 | 0.69 | na | 0.14 | 0.81 | 0.14 | 0.22 |
| 95% CI of | 1.1 | 0.49 | 1.1 | 0.53 | na | 0.80 | 0.36 | 0.023 | 0.68 |
| OR Quart3 | 6.6 | 13 | 8.8 | 2.6 | na | 4.7 | 2.2 | 1.7 | 5.2 |
| OR Quart 4 | 4.2 | 3.0 | 6.3 | 3.4 | 2.5 | 5.0 | 1.3 | 2.0 | 2.1 |
| p Value | 9.4E−4 | 0.18 | 2.2E−4 | 3.4E−4 | 0.12 | 7.1E−5 | 0.53 | 0.20 | 0.16 |
| 95% CI of | 1.8 | 0.61 | 2.4 | 1.8 | 0.79 | 2.3 | 0.56 | 0.68 | 0.76 |
| OR Quart4 | 9.9 | 15 | 17 | 6.8 | 8.2 | 11 | 3.1 | 6.0 | 5.6 |

Immunoglogulin G2

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 8730 | 13800 | 8730 | 16800 | 8730 | 8880 |
| Average | 16700 | 27200 | 16700 | 38600 | 16700 | 23800 |
| Stdev | 28100 | 43200 | 28100 | 60500 | 28100 | 43900 |
| p(t-test) | | 0.0063 | | 2.5E−8 | | 0.13 |
| Min | 119 | 810 | 119 | 1870 | 119 | 25.4 |
| Max | 240000 | 240000 | 240000 | 240000 | 240000 | 240000 |
| n (Samp) | 922 | 62 | 922 | 70 | 922 | 39 |
| n (Patient) | 358 | 62 | 358 | 70 | 358 | 39 |
| sCr only | | | | | | |
| Median | 9310 | 11800 | 9310 | 16700 | 9310 | 40000 |
| Average | 18900 | 26000 | 18900 | 39600 | 18900 | 50100 |
| Stdev | 33500 | 44100 | 33500 | 59100 | 33500 | 64700 |
| p(t-test) | | 0.41 | | 0.010 | | 1.8E−4 |
| Min | 119 | 3150 | 119 | 3190 | 119 | 1310 |
| Max | 240000 | 182000 | 240000 | 240000 | 240000 | 240000 |
| n (Samp) | 1225 | 15 | 1225 | 18 | 1225 | 17 |
| n (Patient) | 438 | 15 | 438 | 18 | 438 | 17 |
| UO only | | | | | | |
| Median | 9320 | 14200 | 9320 | 18200 | 9320 | 9690 |
| Average | 17600 | 34200 | 17600 | 42800 | 17600 | 23600 |
| Stdev | 28600 | 53700 | 28600 | 63300 | 28600 | 43000 |
| p(t-test) | | 9.3E−5 | | 3.9E−9 | | 0.24 |
| Min | 334 | 810 | 334 | 1870 | 334 | 25.4 |
| Max | 240000 | 240000 | 240000 | 240000 | 240000 | 240000 |
| n (Samp) | 810 | 57 | 810 | 63 | 810 | 34 |
| n (Patient) | 280 | 57 | 280 | 63 | 280 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.63 | 0.61 | 0.63 | 0.65 | 0.62 | 0.67 | 0.51 | 0.63 | 0.54 |
| SE | 0.039 | 0.078 | 0.041 | 0.037 | 0.071 | p.038 | 0.047 | 0.073 | 0.052 |
| p | 8.5E−4 | 0.15 | 9.9E−4 | 3.0E−5 | 0.090 | 1.2E−5 | 0.90 | 0.075 | 0.44 |
| nCohort 1 | 922 | 1225 | 810 | 922 | 1225 | 810 | 922 | 1225 | 810 |
| nCohort 2 | 62 | 15 | 57 | 70 | 18 | 63 | 39 | 17 | 34 |
| Cutoff 1 | 8220 | 8900 | 8240 | 8630 | 6340 | 9450 | 5240 | 6690 | 8010 |
| Sens 1 | 71% | 73% | 70% | 70% | 72% | 71% | 72% | 71% | 71% |
| Spec 1 | 47% | 48% | 45% | 50% | 33% | 51% | 28% | 36% | 44% |
| Cutoff 2 | 6900 | 8400 | 6900 | 5620 | 4630 | 6460 | 4240 | 4240 | 4620 |
| Sens 2 | 81% | 80% | 81% | 80% | 83% | 81% | 82% | 82% | 82% |
| Spec 2 | 39% | 45% | 38% | 31% | 22% | 35% | 21% | 20% | 21% |
| Cutoff 3 | 4910 | 5870 | 4400 | 3990 | 3980 | 4130 | 1800 | 1800 | 2270 |
| Sens 3 | 90% | 93% | 91% | 90% | 94% | 90% | 92% | 94% | 91% |
| Spec 3 | 25% | 30% | 20% | 20% | 18% | 19% | 6% | 5% | 8% |
| Cutoff 4 | 14400 | 15700 | 14900 | 14400 | 15700 | 14900 | 14400 | 15700 | 14900 |
| Sens 4 | 45% | 40% | 47% | 53% | 50% | 54% | 28% | 59% | 32% |
| Spec 4 | 70% | 70% | 70% | 70% | 70% | 70% | 70% | 70% | 70% |
| Cutoff 5 | 19700 | 21200 | 20100 | 19700 | 21200 | 20100 | 19700 | 21200 | 20100 |
| Sens 5 | 44% | 33% | 40% | 43% | 44% | 43% | 23% | 59% | 26% |
| Spec 5 | 80% | 80% | 80% | 80% | 80% | 80% | 80% | 80% | 80% |
| Cutoff 6 | 32100 | 37400 | 35300 | 32100 | 37400 | 35300 | 32100 | 37400 | 35300 |
| Sens 6 | 19% | 7% | 25% | 31% | 28% | 30% | 18% | 53% | 15% |
| Spec 6 | 90% | 90% | 90% | 90% | 90% | 90% | 90% | 90% | 90% |
| OR Quart 2 | 2.5 | 5.1 | 1.3 | 0.91 | 0.75 | 1.1 | 0.63 | 0.75 | 1.0 |
| p Value | 0.043 | 0.14 | 0.51 | 0.83 | 0.70 | 0.81 | 0.34 | 0.70 | 1.0 |
| 95% CI of | 1.0 | 0.59 | 0.56 | 0.39 | 0.17 | 0.44 | 0.24 | 0.17 | 0.37 |
| OR Quart2 | 6.2 | 44 | 3.3 | 2.1 | 3.4 | 2.8 | 1.6 | 3.4 | 2.7 |
| OR Quart 3 | 1.6 | 3.0 | 1.1 | 1.1 | 0.50 | 1.5 | 1.1 | 0 | 1.1 |
| p Value | 0.34 | 0.34 | 0.82 | 0.84 | 0.42 | 0.38 | 0.83 | na | 0.80 |
| 95% CI of | 0.61 | 0.31 | 0.44 | 0.49 | 0.090 | 0.62 | 0.47 | na | 0.43 |
| OR Quart3 | 4.2 | 29 | 2.8 | 2.4 | 2.7 | 3.5 | 2.5 | na | 3.0 |
| OR Quart 4 | 4.2 | 6.1 | 3.1 | 3.1 | 2.3 | 3.8 | 0.81 | 2.5 | 1.1 |
| p Value | 9.4E−4 | 0.095 | 0.0043 | 0.0011 | 0.17 | 6.1E−4 | 0.64 | 0.12 | 0.80 |
| 95% CI of | 1.8 | 0.73 | 1.4 | 1.6 | 0.69 | 1.8 | 0.33 | 0.79 | 0.43 |
| OR Quart4 | 9.9 | 51 | 6.9 | 6.2 | 7.5 | 8.3 | 2.0 | 8.2 | 3.0 |

Interleukin-11

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 129 | 241 | 129 | 212 | 129 | 120 |
| Average | 209 | 341 | 209 | 288 | 209 | 207 |
| Stdev | 243 | 387 | 243 | 334 | 243 | 338 |
| p(t-test) | | 8.2E−5 | | 0.012 | | 0.97 |
| Min | 0.0822 | 21.2 | 0.0822 | 28.3 | 0.0822 | 14.5 |
| Max | 2260 | 2900 | 2260 | 2590 | 2260 | 2140 |
| n (Samp) | 929 | 62 | 929 | 69 | 929 | 39 |
| n (Patient) | 361 | 62 | 361 | 69 | 361 | 39 |
| sCr only | | | | | | |
| Median | 135 | 158 | 135 | 224 | 135 | 174 |
| Average | 231 | 256 | 231 | 258 | 231 | 192 |
| Stdev | 303 | 226 | 303 | 144 | 303 | 127 |
| p(t-test) | | 0.75 | | 0.71 | | 0.59 |
| Min | 0.0822 | 21.2 | 0.0822 | 32.3 | 0.0822 | 35.3 |
| Max | 3780 | 830 | 3780 | 547 | 3780 | 549 |
| n (Samp) | 1232 | 15 | 1232 | 18 | 1232 | 17 |
| n (Patient) | 441 | 15 | 441 | 18 | 441 | 17 |
| UO only | | | | | | |
| Median | 139 | 285 | 139 | 210 | 139 | 121 |
| Average | 217 | 374 | 217 | 307 | 217 | 221 |
| Stdev | 246 | 416 | 246 | 374 | 246 | 360 |
| p(t-test) | | 1.2E−5 | | 0.0083 | | 0.94 |
| Min | 0.0822 | 27.2 | 0.0822 | 28.3 | 0.0822 | 14.5 |
| Max | 2260 | 2900 | 2260 | 2590 | 2260 | 2140 |
| n (Samp) | 817 | 57 | 817 | 62 | 817 | 34 |
| n (Patient) | £83 | 57 | 283 | 62 | 283 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.68 | 0.58 | 0.69 | 0.64 | 0.65 | 0.62 | 0.50 | 0.54 | 0.49 |
| SE | 0.038 | 0.078 | 0.040 | 0.037 | 0.071 | 0.039 | 0.047 | 0.072 | 0.051 |
| p | 2.4E−6 | 0.33 | 2.1E−6 | 1.6E−4 | 0.033 | 0.0015 | 0.95 | 0.55 | 0.79 |
| nCohort 1 | 929 | 1232 | 817 | 929 | 1232 | 817 | 929 | 1232 | 817 |
| nCohort 2 | 62 | 15 | 57 | 69 | 18 | 62 | 39 | 17 | 34 |
| Cutoff 1 | 149 | 136 | 158 | 123 | 182 | 123 | 88.1 | 135 | 88.1 |
| Sens 1 | 71% | 73% | 70% | 71% | 72% | 71% | 72% | 71% | 71% |
| Spec 1 | 57% | 50% | 56% | 49% | 62% | 45% | 35% | 50% | 31% |
| Cutoff 2 | 119 | 87.1 | 128 | 101 | 105 | 101 | 71.2 | 93.8 | 74.7 |
| Sens 2 | 81% | 80% | 81% | 81% | 83% | 81% | 82% | 82% | 82% |
| Spec 2 | 47% | 32% | 46% | 40% | 39% | 37% | 26% | 35% | 24% |
| Cutoff 3 | 79.8 | 63.1 | 85.3 | 76.3 | 89.4 | 79.6 | 54.9 | 41.4 | 62.9 |
| Sens 3 | 90% | 93% | 91% | 91% | 94% | 90% | 92% | 94% | 91% |
| Spec 3 | 31% | 20% | 29% | 28% | 33% | 28% | 18% | 10% | 19% |
| Cutoff 4 | 212 | 230 | 230 | 212 | 230 | 230 | 212 | 230 | 230 |
| Sens 4 | 55% | 33% | 54% | 51% | 44% | 45% | 26% | 29% | 26% |
| Spec 4 | 70% | 70% | 70% | 70% | 70% | 70% | 70% | 70% | 70% |
| Cutoff 5 | 301 | 328 | 307 | 301 | 328 | 307 | 301 | 328 | 307 |
| Sens 5 | 42% | 33% | 44% | 33% | 33% | 34% | 13% | 12% | 15% |
| Spec 5 | 80% | 80% | 80% | 80% | 80% | 80% | 80% | 80% | 80% |
| Cutoff 6 | 461 | 509 | 460 | 461 | 509 | 460 | 461 | 509 | 460 |
| Sens 6 | 26% | 13% | 28% | 13% | 6% | 15% | 5% | 6% | 6% |
| Spec 6 | 90% | 90% | 90% | 90% | 90% | 90% | 90% | 90% | 90% |
| OR Quart 2 | 2.8 | 1.00 | 4.2 | 2.6 | 3.0 | 2.8 | 1.6 | 1.0 | 1.3 |
| p Value | 0.079 | 1.00 | 0.029 | 0.053 | 0.34 | 0.036 | 0.34 | 1.0 | 0.59 |
| 95% CI of | 0.89 | 0.14 | 1.2 | 0.99 | 0.31 | 1.1 | 0.61 | 0.20 | 0.46 |
| OR Quart2 | 9.0 | 7.1 | 15 | 6.8 | 29 | 7.3 | 4.2 | 5.0 | 3.9 |
| OR Quart 3 | 4.8 | 3.0 | 4.5 | 3.7 | 8.2 | 3.0 | 2.1 | 2.4 | 2.2 |
| p Value | 0.0054 | 0.18 | 0.019 | 0.0053 | 0.048 | 0.025 | 0.13 | 0.22 | 0.11 |
| 95% CI of | 1.6 | 0.61 | 1.3 | 1.5 | 1.0 | 1.1 | 0.82 | 0.61 | 0.84 |
| OR Quart3 | 14 | 15 | 16 | 9.4 | 66 | 7.7 | 5.2 | 9.2 | 6.0 |
| OR Quart 4 | 8.0 | 2.5 | 11 | 4.9 | 6.1 | 4.1 | 1.0 | 1.3 | 1.2 |
| p Value | 1 .2E−4 | 0.27 | 9.9E−5 | 5.6E−4 | 0.096 | 0.0024 | 1.0 | 0.71 | 0.77 |
| 95% CI of | 2.8 | 0.48 | 3.3 | 2.0 | 0.73 | 1.7 | 0.35 | 0.30 | 0.39 |
| OR Quart4 | 23 | 13 | 36 | 12 | 51 | 10 | 2.9 | 6.0 | 3.6 |

Interleukin-2 receptor alpha chain

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 554 | 923 | 554 | 868 | 554 | 524 |
| Average | 909 | 1440 | 909 | 1430 | 909 | 1190 |
| Stdev | 1090 | 1520 | 1090 | 1780 | 1090 | 1700 |
| p(t-test) | | 3.1E−4 | | 3.0E−4 | | 0.12 |
| Min | 0.0317 | 0.0482 | 0.0317 | 0.0482 | 0.0317 | 0.0383 |
| Max | 10400 | 6080 | 10400 | 9090 | 10400 | 7740 |
| n (Samp) | 930 | 62 | 930 | 70 | 930 | 39 |
| n (Patient) | 361 | 62 | 361 | 70 | 361 | 39 |
| sCr only | | | | | | |
| Median | 621 | 499 | 621 | 957 | 621 | 421 |
| Average | 1100 | 1150 | 1100 | 1680 | 1100 | 881 |
| Stdev | 1420 | 1520 | 1420 | 1930 | 1420 | 923 |
| p(t-test) | | 0.89 | | 0.086 | | 0.54 |
| Min | 0.0317 | 0.0482 | 0.0317 | 0.0482 | 0.0317 | 51.4 |
| Max | 10400 | 6080 | 10400 | 7190 | 10400 | 2590 |
| n (Samp) | 1234 | 15 | 1234 | 18 | 1234 | 17 |
| n (Patient) | 441 | 15 | 441 | 18 | 441 | 17 |
| UO only | | | | | | |
| Median | 563 | 1080 | 563 | 911 | 563 | 728 |
| Average | 927 | 1640 | 927 | 1570 | 927 | 1380 |
| Stdev | 1110 | 1540 | 1110 | 1840 | 1110 | 1780 |
| p(t-test) | | 5.2E−6 | | 2.7E−5 | | 0.022 |
| Min | 0.0317 | 0.249 | 0.0317 | 0.0632 | 0.0317 | 0.0383 |
| Max | 10400 | 6080 | 10400 | 9090 | 10400 | 7740 |
| n (Samp) | 819 | 57 | 819 | 63 | 819 | 34 |
| n (Patient) | 283 | 57 | 283 | 63 | 283 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.61 | 0.51 | 0.66 | 0.60 | 0.58 | 0.62 | 0.51 | 0.48 | 0.55 |
| SE | 0.039 | 0.076 | 0.040 | 0.037 | 0.071 | 0.039 | 0.048 | 0.071 | 0.052 |
| p | 0.0042 | 0.85 | 1.0E−4 | 0.0093 | 0.24 | 0.0014 | 0.78 | 0.75 | 0.31 |
| nCohort 1 | 930 | 1234 | 819 | 930 | 1234 | 819 | 930 | 1234 | 819 |
| nCohort 2 | 62 | 15 | 57 | 70 | 18 | 63 | 39 | 17 | 34 |
| Cutoff 1 | 446 | 411 | 528 | 417 | 373 | 466 | 226 | 226 | 333 |
| Sens 1 | 71% | 73% | 70% | 70% | 72% | 71% | 72% | 71% | 71% |
| Spec 1 | 45% | 40% | 48% | 44% | 37% | 45% | 31% | 27% | 36% |
| Cutoff 2 | 278 | 277 | 368 | 272 | 235 | 318 | 135 | 135 | 189 |
| Sens 2 | 81% | 80% | 81% | 80% | 83% | 81% | 82% | 82% | 82% |
| Spec 2 | 35% | 31% | 39% | 34% | 28% | 35% | 21% | 18% | 26% |
| Cutoff 3 | 112 | 21.0 | 167 | 109 | 55.0 | 152 | 35.7 | 66.8 | 35.7 |
| Sens 3 | 90% | 93% | 91% | 90% | 94% | 90% | 92% | 94% | 91% |
| Spec 3 | 18% | 8% | 24% | 17% | 11% | 23% | 11% | 12% | 10% |
| Cutoff 4 | 1050 | 1150 | 1060 | 1050 | 1150 | 1060 | 1050 | 1150 | 1060 |
| Sens 4 | 44% | 40% | 51% | 39% | 50% | 41% | 28% | 24% | 35% |
| Spec 4 | 70% | 70% | 70% | 70% | 70% | 70% | 70% | 70% | 70% |
| Cutoff 5 | 1490 | 1740 | 1500 | 1490 | 1740 | 1500 | 1490 | 1740 | 1500 |
| Sens 5 | 34% | 13% | 40% | 31% | 33% | 35% | 21% | 24% | 29% |
| Spec 5 | 80% | 80% | 80% | 80% | 80% | 80% | 80% | 80% | 80% |
| Cutoff 6 | 2250 | 2620 | 2290 | 2250 | 2620 | 2290 | 2250 | 2620 | 2290 |
| Sens 6 | 23% | 7% | 28% | 17% | 17% | 21% | 18% | 0% | 21% |
| Spec 6 | 90% | 90% | 90% | 90% | 90% | 90% | 90% | 90% | 90% |
| OR Quart 2 | 2.3 | 1.7 | 2.2 | 1.9 | 1.3 | 2.4 | 1.2 | 0.75 | 1.7 |
| p Value | 0.050 | 0.48 | 0.11 | 0.12 | 0.70 | 0.062 | 0.65 | 0.70 | 0.31 |
| 95% CI of | 1.0 | 0.40 | 0.84 | 0.84 | 0.30 | 0.96 | 0.50 | 0.17 | 0.61 |
| OR Quart2 | 5.5 | 7.1 | 6.0 | 4.1 | 6.0 | 5.9 | 3.0 | 3.4 | 4.8 |
| OR Quart 3 | 1.7 | 0.66 | 2.2 | 2.0 | 1.0 | 2.7 | 1.0 | 1.5 | 1.0 |
| p Value | 0.27 | 0.66 | 0.11 | 0.090 | 1.0 | 0.029 | 1.0 | 0.53 | 1.0 |
| 95% CI of | 0.68 | 0.11 | 0.84 | 0.90 | 0.20 | 1.1 | 0.39 | 0.42 | 0.32 |
| OR Quart3 | 4.1 | 4.0 | 6.0 | 4.3 | 5.0 | 6.6 | 2.6 | 5.4 | 3.2 |
| OR Quart 4 | 3.1 | 1.7 | 4.6 | 2.4 | 2.7 | 3.4 | 1.1 | 1.0 | 2.0 |
| p Value | 0.0077 | 0.48 | 0.0011 | 0.023 | 0.14 | 0.0064 | 0.82 | 1.00 | 0.16 |
| 95% CI of | 1.3 | 0.40 | 1.8 | 1.1 | 0.71 | 1.4 | 0.44 | 0.25 | 0.75 |
| OR Quart4 | 7.0 | 7.1 | 11 | 5.2 | 10 | 8.0 | 2.8 | 4.0 | 5.6 |

Neutrophil collagenase

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 3070 | 6050 | 3070 | 8280 | 3070 | 3260 |
| Average | 13400 | 18500 | 13400 | 34000 | 13400 | 9350 |
| Stdev | 38800 | 30300 | 38800 | 83200 | 38800 | 15200 |
| p(t-test) | | 0.29 | | 5.1E−5 | | 0.48 |
| Min | 0.114 | 0.159 | 0.114 | 25.8 | 0.114 | 111 |
| Max | 670000 | 136000 | 670000 | 670000 | 670000 | 66900 |
| n (Samp) | 1005 | 69 | 1005 | 80 | 1005 | 45 |
| n (Patient) | 387 | 69 | 387 | 80 | 387 | 45 |
| sCr only | | | | | | |
| Median | 3570 | 4670 | 3570 | 6260 | 3570 | 5710 |
| Average | 14700 | 20300 | 14700 | 69300 | 14700 | 17200 |
| Stdev | 41000 | 40500 | 41000 | 157000 | 41000 | 27500 |
| p(t-test) | | 0.59 | | 4.2E−8 | | 0.79 |
| Min | 0.114 | 0.260 | 0.114 | 25.8 | 0.114 | 111 |
| Max | 670000 | 128000 | 670000 | 625000 | 670000 | 115000 |
| n (Samp) | 1339 | 16 | 1339 | 21 | 1339 | 19 |
| n (Patient) | 473 | 16 | 473 | 21 | 473 | 19 |
| UO only | | | | | | |
| Median | 3120 | 7870 | 3120 | 10400 | 3120 | 3470 |
| Average | 13700 | 31600 | 13700 | 42800 | 13700 | 10000 |
| Stdev | 35000 | 78300 | 35000 | 94000 | 35000 | 15600 |
| p(t-test) | | 4.5E−4 | | 2.0E−8 | | 0.51 |
| Min | 0.114 | 0.159 | 0.114 | 0.260 | 0.114 | 25.8 |
| Max | 365000 | 561000 | 365000 | 670000 | 365000 | 66900 |
| n (Samp) | 896 | 64 | 896 | 73 | 896 | 40 |
| n (Patient) | 311 | 64 | 311 | 73 | 311 | 40 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.64 | 0.52 | 0.67 | 0.66 | 0.57 | 0.70 | 0.50 | 0.57 | 0.52 |
| SE | 0.037 | 0.074 | 0.038 | 0.034 | 0.065 | 0.035 | 0.044 | 0.069 | 0.047 |
| p | 1.9E−4 | 0.75 | 1.2E−5 | 2.1E−6 | 0.32 | 2.4E−8 | 0.92 | 0.34 | 0.70 |
| nCohort 1 | 1005 | 1339 | 896 | 1005 | 1339 | 896 | 1005 | 1339 | 896 |
| nCohort 2 | 69 | 16 | 64 | 80 | 21 | 73 | 45 | 19 | 40 |
| Cutoff 1 | 3000 | 1060 | 3260 | 3320 | 1020 | 4640 | 1270 | 2660 | 1680 |
| Sens 1 | 71% | 75% | 70% | 70% | 71% | 71% | 71% | 74% | 70% |
| Spec 1 | 50% | 23% | 51% | 52% | 22% | 59% | 30% | 43% | 36% |
| Cutoff 2 | 2400 | 1050 | 2830 | 2190 | 978 | 3080 | 742 | 751 | 813 |
| Sens 2 | 81% | 81% | 81% | 80% | 81% | 81% | 80% | 84% | 80% |
| Spec 2 | 45% | 23% | 48% | 43% | 22% | 50% | 20% | 18% | 21% |
| Cutoff 3 | 1050 | 358 | 1720 | 962 | 570 | 1550 | 387 | 191 | 571 |
| Sens 3 | 91% | 94% | 91% | 90% | 90% | 90% | 91% | 95% | 90% |
| Spec 3 | 25% | 9% | 36% | 24% | 14% | 34% | 12% | 4% | 16% |
| Cutoff 4 | 7950 | 9120 | 7860 | 7950 | 9120 | 7860 | 7950 | 9120 | 7860 |
| Sens 4 | 45% | 38% | 50% | 52% | 43% | 58% | 27% | 47% | 30% |
| Spec 4 | 70% | 70% | 70% | 70% | 70% | 70% | 70% | 70% | 70% |
| Cutoff 5 | 13800 | 15700 | 13900 | 13800 | 15700 | 13900 | 13800 | 15700 | 13900 |
| Sens 5 | 33% | 19% | 36% | 39% | 33% | 41% | 20% | 32% | 22% |
| Spec 5 | 80% | 80% | 80% | 80% | 80% | 80% | 80% | 80% | 80% |
| Cutoff 6 | 29000 | 32400 | 30900 | 29000 | 32400 | 30900 | 29000 | 32400 | 30900 |
| Sens 6 | 17% | 12% | 19% | 26% | 24% | 29% | 9% | 16% | 10% |
| Spec 6 | 90% | 90% | 90% | 90% | 90% | 90% | 90% | 90% | 90% |
| OR Quart 2 | 2.4 | 0.40 | 4.2 | 1.2 | 0.28 | 2.2 | 0.92 | 0.59 | 1.0 |
| p Value | 0.063 | 0.27 | 0.011 | 0.68 | 0.12 | 0.11 | 0.84 | 0.48 | 1.0 |
| 95% CI of | 0.95 | 0.076 | 1.4 | 0.52 | 0.058 | 0.83 | 0.40 | 0.14 | 0.39 |
| OR Quart2 | 5.8 | 2.1 | 13 | 2.7 | 1.4 | 6.0 | 2.1 | 2.5 | 2.6 |
| OR Quart 3 | 2.7 | 0.80 | 4.2 | 1.9 | 0.42 | 3.4 | 0.83 | 0.80 | 1.1 |
| p Value | 0.030 | 0.73 | 0.011 | 0.10 | 0.22 | 0.011 | 0.66 | 0.74 | 0.81 |
| 95% CI of | 1.1 | 0.21 | 1.4 | 0.88 | 0.11 | 1.3 | 0.35 | 0.21 | 0.45 |
| OR Quart3 | 6.5 | 3.0 | 13 | 4.0 | 1.7 | 8.5 | 1.9 | 3.0 | 2.8 |
| OR Quart 4 | 4.3 | 1.00 | 7.8 | 3.6 | 1.3 | 6.6 | 1.0 | 1.4 | 1.4 |
| p Value | 6.9E−4 | 1.00 | 1.6E−4 | 3.2E−4 | 0.61 | 2.9E−5 | 0.99 | 0.57 | 0.50 |
| 95% CI of | 1.9 | 0.29 | 2.7 | 1.8 | 0.48 | 2.7 | 0.44 | 0.44 | 0.56 |
| OR Quart4 | 10 | 3.5 | 23 | 7.2 | 3.5 | 16 | 2.3 | 4.5 | 3.3 |

Protransforming growth factor alpha

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 5.81 | 8.51 | 5.81 | 10.3 | 5.81 | 6.15 |
| Average | 10.7 | 12.5 | 10.7 | 16.0 | 10.7 | 9.77 |
| Stdev | 22.4 | 12.1 | 22.4 | 19.8 | 22.4 | 10.6 |
| p(t-test) | | 0.54 | | 0.057 | | 0.80 |
| Min | 0.00184 | 0.00603 | 0.00184 | 0.00603 | 0.00184 | 0.00454 |
| Max | 361 | 58.8 | 361 | 135 | 361 | 52.3 |
| n (Samp) | 925 | 62 | 925 | 69 | 925 | 39 |
| n (Patient) | 361 | 62 | 361 | 69 | 361 | 39 |
| sCr only | | | | | | |
| Median | 6.55 | 12.3 | 6.55 | 13.2 | 6.55 | 6.54 |
| Average | 11.4 | 11.8 | 11.4 | 16.9 | 11.4 | 10.6 |
| Stdev | 21.7 | 6.57 | 21.7 | 16.6 | 21.7 | 11.4 |
| p(t-test) | | 0.93 | | 0.28 | | 0.89 |
| Min | 0.00184 | 0.00603 | 0.00184 | 0.00603 | 0.00184 | 0.00574 |
| Max | 361 | 27.7 | 361 | 59.6 | 361 | 45.2 |
| n (Samp) | 1228 | 15 | 1228 | 18 | 1228 | 17 |
| n (Patient) | 441 | 15 | 441 | 18 | 441 | 17 |
| UO only | | | | | | |
| Median | 5.87 | 8.48 | 5.87 | 10.9 | 5.87 | 6.31 |
| Average | 11.1 | 13.7 | 11.1 | 16.6 | 11.1 | 10.8 |
| Stdev | 23.7 | 14.0 | 23.7 | 19.7 | 23.7 | 11.4 |
| p(t-test) | | 0.42 | | 0.075 | | 0.94 |
| Min | 0.00184 | 0.0114 | 0.00184 | 0.781 | 0.00184 | 0.00454 |
| Max | 361 | 63.1 | 361 | 135 | 361 | 52.3 |
| n (Samp) | 814 | 57 | 814 | 62 | 814 | 34 |
| n (Patient) | 283 | 57 | 283 | 62 | 283 | 34 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.62 | 0.66 | 0.63 | 0.65 | 0.63 | 0.67 | 0.53 | 0.53 | 0.54 |
| SE | 0.039 | 0.077 | 0.041 | 0.037 | 0.071 | 0.039 | 0.048 | 0.072 | 0.052 |
| p | 0.0018 | 0.035 | 0.0012 | 8.3E−5 | 0.071 | 6.1E−6 | 0.57 | 0.71 | 0.40 |
| nCohort 1 | 925 | 1228 | 814 | 925 | 1228 | 814 | 925 | 1228 | 814 |
| nCohort 2 | 62 | 15 | 57 | 69 | 18 | 62 | 39 | 17 | 34 |
| Cutoff 1 | 6.53 | 9.29 | 6.56 | 6.12 | 4.91 | 7.51 | 3.64 | 5.14 | 3.71 |
| Sens 1 | 71% | 73% | 70% | 71% | 72% | 71% | 72% | 71% | 71% |
| Spec 1 | 54% | 66% | 54% | 51% | 39% | 61% | 31% | 41% | 31% |
| Cutoff 2 | 5.18 | 7.12 | 5.42 | 4.16 | 3.53 | 5.57 | 2.57 | 2.47 | 3.00 |
| Sens 2 | 81% | 80% | 81% | 81% | 83% | 81% | 82% | 82% | 82% |
| Spec 2 | 45% | 54% | 47% | 36% | 28% | 48% | 21% | 18% | 25% |
| Cutoff 3 | 2.06 | 3.69 | 2.04 | 2.10 | 2.84 | 2.37 | 1.71 | 1.85 | 0.530 |
| Sens 3 | 90% | 93% | 91% | 91% | 94% | 90% | 92% | 94% | 91% |
| Spec 3 | 17% | 29% | 16% | 17% | 22% | 19% | 14% | 14% | 5% |
| Cutoff 4 | 9.53 | 10.3 | 9.60 | 9.53 | 10.3 | 9.60 | 9.53 | 10.3 | 9.60 |
| Sens 4 | 42% | 60% | 44% | 55% | 56% | 58% | 41% | 29% | 44% |
| Spec 4 | 70% | 70% | 70% | 70% | 70% | 70% | 70% | 70% | 70% |
| Cutoff 5 | 12.4 | 14.0 | 12.4 | 12.4 | 14.0 | 12.4 | 12.4 | 14.0 | 12.4 |
| Sens 5 | 34% | 40% | 37% | 39% | 44% | 42% | 28% | 24% | 32% |
| Spec 5 | 80% | 80% | 80% | 80% | 80% | 80% | 80% | 80% | 80% |
| Cutoff 6 | 20.3 | 23.5 | 21.6 | 20.3 | 23.5 | 21.6 | 20.3 | 23.5 | 21.6 |
| Sens 6 | 16% | 7% | 16% | 20% | 28% | 21% | 10% | 12% | 12% |
| Spec 6 | 90% | 90% | 90% | 90% | 90% | 90% | 90% | 90% | 90% |
| OR Quart 2 | 1.00 | 2.0 | 1.00 | 1.3 | 1.7 | 1.4 | 1.3 | 1.3 | 1.3 |
| p Value | 0.99 | 0.57 | 0.99 | 0.51 | 0.48 | 0.46 | 0.63 | 0.74 | 0.61 |
| 95% CI of | 0.37 | 0.18 | 0.34 | 0.56 | 0.40 | 0.54 | 0.49 | 0.33 | 0.47 |
| OR Quart2 | 2.7 | 22 | 2.9 | 3.3 | 7.1 | 3.9 | 3.3 | 4.7 | 3.6 |
| OR Quart 3 | 3.1 | 4.0 | 3.2 | 1.8 | 0 | 2.1 | 1.1 | 0.75 | 0.71 |
| p Value | 0.0080 | 0.21 | 0.0094 | 0.16 | na | 0.12 | 0.81 | 0.70 | 0.56 |
| 95% CI of | 1.3 | 0.45 | 1.3 | 0.79 | na | 0.82 | 0.43 | 0.17 | 0.22 |
| OR Quart3 | 7.0 | 36 | 7.7 | 4.2 | na | 5.2 | 3.0 | 3.4 | 2.3 |
| OR Quart 4 | 3.1 | 8.2 | 3.4 | 3.9 | 3.4 | 5.0 | 1.5 | 1.2 | 1.9 |
| p Value | 0.0080 | 0.048 | 0.0064 | 4.5E−4 | 0.065 | 1.9E−4 | 0.36 | 0.74 | 0.18 |
| 95% CI of | 1.3 | 1.0 | 1.4 | 1.8 | 0.93 | 2.1 | 0.61 | 0.33 | 0.75 |
| OR Quart4 | 7.0 | 66 | 8.1 | 8.4 | 12 | 12 | 3.8 | 4.7 | 4.9 |

CA 15-3

| | 0 hr prior to AKI stage | | 24 hr prior to AKI stage | | 48 hr prior to AKI stage | |
|---|---|---|---|---|---|---|
| | Cohort 1 | Cohort 2 | Cohort 1 | Cohort 2 | Cohort 1 | Cohort 2 |
| sCr or UO | | | | | | |
| Median | 434 | 434 | 434 | 434 | 434 | 434 |
| Average | 314 | 393 | 314 | 352 | 314 | 329 |
| Stdev | 236 | 116 | 236 | 212 | 236 | 171 |
| p(t-test) | | 0.041 | | 0.28 | | 0.75 |
| Min | 2.21 | 1.17 | 2.21 | 21.3 | 2.21 | 4.72 |
| Max | 784 | 434 | 784 | 784 | 784 | 434 |
| n (Samp) | 407 | 39 | 407 | 50 | 407 | 27 |
| n (Patient) | 214 | 39 | 214 | 50 | 214 | 27 |
| sCr only | | | | | | |
| Median | nd | nd | 434 | 434 | 434 | 434 |
| Average | nd | nd | 329 | 350 | 329 | 351 |
| Stdev | nd | nd | 223 | 223 | 223 | 152 |
| p(t-test) | nd | nd | | 0.78 | | 0.75 |
| Min | nd | nd | 1.17 | 39.8 | 1.17 | 43.5 |
| Max | nd | nd | 784 | 784 | 784 | 434 |
| n (Samp) | nd | nd | 535 | 10 | 535 | 11 |
| n (Patient) | nd | nd | 256 | 10 | 256 | 11 |
| UO only | | | | | | |
| Median | 434 | 434 | 434 | 434 | 434 | 434 |
| Average | 316 | 394 | 316 | 375 | 316 | 327 |
| Stdev | 232 | 116 | 232 | 215 | 232 | 177 |
| p(t-test) | | 0.039 | | 0.095 | | 0.81 |
| Min | 2.21 | 1.17 | 2.21 | 21.3 | 2.21 | 4.72 |
| Max | 784 | 434 | 784 | 784 | 784 | 434 |
| n (Samp) | 380 | 39 | 380 | 47 | 380 | 25 |
| n (Patient) | 189 | 39 | 189 | 47 | 189 | 25 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.60 | nd | 0.60 | 0.57 | 0.52 | 0.59 | 0.54 | 0.54 | 0.53 |
| SE | 0.050 | nd | 0.050 | 0.044 | 0.094 | 0.046 | 0.058 | 0.090 | 0.061 |
| p | 0.055 | nd | 0.048 | 0.11 | 0.80 | 0.056 | 0.54 | 0.62 | 0.60 |
| nCohort 1 | 407 | nd | 380 | 407 | 535 | 380 | 407 | 535 | 380 |
| nCohort 2 | 39 | nd | 39 | 50 | 10 | 47 | 27 | 11 | 25 |
| Cutoff 1 | 427 | nd | 427 | 239 | 265 | 427 | 427 | 427 | 427 |
| Sens 1 | 82% | nd | 85% | 70% | 70% | 70% | 70% | 73% | 72% |
| Spec 1 | 43% | nd | 42% | 39% | 35% | 42% | 43% | 39% | 42% |
| Cutoff 2 | 427 | nd | 427 | 80.0 | 167 | 87.5 | 75.8 | 265 | 75.8 |
| Sens 2 | 82% | nd | 85% | 80% | 80% | 81% | 81% | 82% | 80% |
| Spec 2 | 43% | nd | 42% | 31% | 32% | 30% | 30% | 35% | 29% |
| Cutoff 3 | 265 | nd | 265 | 43.7 | 45.0 | 42.6 | 18.9 | 75.8 | 18.8 |
| Sens 3 | 92% | nd | 92% | 90% | 90% | 91% | 93% | 91% | 92% |
| Spec 3 | 40% | nd | 39% | 22% | 20% | 22% | 12% | 26% | 11% |
| Cutoff 4 | 434 | nd | 434 | 434 | 434 | 434 | 434 | 434 | 434 |
| Sens 4 | 0% | nd | 0% | 12% | 10% | 15% | 0% | 0% | 0% |
| Spec 4 | 90% | nd | 91% | 90% | 91% | 91% | 90% | 91% | 91% |
| Cutoff 5 | 434 | nd | 434 | 434 | 434 | 434 | 434 | 434 | 434 |
| Sens 5 | 0% | nd | 0% | 12% | 10% | 15% | 0% | 0% | 0% |
| Spec 5 | 90% | nd | 91% | 90% | 91% | 91% | 90% | 91% | 91% |
| Cutoff 6 | 434 | nd | 434 | 434 | 434 | 434 | 434 | 434 | 434 |
| Sens 6 | 0% | nd | 0% | 12% | 10% | 15% | 0% | 0% | 0% |
| Spec 6 | 90% | nd | 91% | 90% | 91% | 91% | 90% | 91% | 91% |
| OR Quart 2 | 8.3 | nd | 11 | 1.0 | 3.6 | 1.3 | 0.99 | 11 | 1.0 |
| p Value | 8.3E−4 | nd | 1.3E−4 | 1.0 | 0.11 | 0.59 | 0.99 | 0.025 | 1.0 |
| 95% CI of | 2.4 | nd | 3.2 | 0.36 | 0.74 | 0.45 | 0.24 | 1.3 | 0.24 |
| OR Quart2 | 29 | nd | 38 | 2.8 | 18 | 4.0 | 4.1 | 84 | 4.1 |
| OR Quart 3 | 5.6 | nd | 3.5 | 4.3 | 0 | 5.3 | 5.6 | 0 | 4.9 |
| p Value | 0.0077 | nd | 0.060 | 6.1E−4 | na | 4.4E−4 | 0.0026 | na | 0.0057 |
| 95% CI of | 1.6 | nd | 0.95 | 1.9 | na | 2.1 | 1.8 | na | 1.6 |
| OR Quart3 | 20 | nd | 13 | 9.9 | na | 14 | 17 | na | 15 |
| OR Quart 4 | 0 | nd | 0 | 0.73 | 0.49 | 1.2 | 0 | 0 | 0 |
| p Value | na | nd | na | 0.57 | 0.57 | 0.79 | na | na | na |
| 95% CI of | na | nd | na | 0.24 | 0.044 | 0.38 | na | na | na |
| OR Quart4 | na | nd | na | 2.2 | 5.5 | 3.6 | na | na | na |

TABLE 3
Comparison of marker levels in urine samples collected within 12 hours of reaching
stage R from Cohort 1 (patients that reached, but did not progress beyond, RIFLE
stage R) and from Cohort 2 (patients that reached RIFLE stage I or F).
C-C motif chemokine 8

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 1.72 | 4.28 | 1.72 | 3.74 | 1.72 | 4.29 |
| Average | 11.4 | 14.2 | 20.6 | 8.92 | 10.2 | 10.3 |
| Stdev | 46.1 | 35.5 | 47.4 | 11.7 | 48.3 | 20.5 |
| p(t-test) | | 0.71 | | 0.37 | | 1.00 |
| Min | 0.0635 | 0.0250 | 0.0635 | 0.206 | 0.0701 | 0.0250 |
| Max | 473 | 214 | 214 | 40.5 | 473 | 108 |
| n (Samp) | 124 | 45 | 49 | 14 | 97 | 31 |
| n (Patient) | 124 | 45 | 49 | 14 | 97 | 31 |

| | At Enrollment | | |
|---|---|---|---|
| | sCr or UO | sCr only | UO only |
| AUC | 0.58 | 0.56 | 0.57 |
| SE | 0.051 | 0.089 | 0.060 |
| p | 0.097 | 0.48 | 0.22 |
| nCohort 1 | 124 | 49 | 97 |
| nCohort 2 | 45 | 14 | 31 |
| Cutoff 1 | 0.829 | 1.21 | 0.746 |
| Sens 1 | 71% | 79% | 74% |
| Spec 1 | 38% | 45% | 34% |
| Cutoff 2 | 0.627 | 0.756 | 0.555 |
| Sens 2 | 80% | 86% | 81% |
| Spec 2 | 35% | 33% | 34% |
| Cutoff 3 | 0.136 | 0.306 | 0.0775 |
| Sens 3 | 91% | 93% | 90% |
| Spec 3 | 19% | 22% | 6% |
| Cutoff 4 | 4.86 | 9.22 | 4.72 |
| Sens 4 | 44% | 29% | 48% |
| Spec 4 | 70% | 71% | 70% |
| Cutoff 5 | 8.21 | 19.7 | 7.85 |
| Sens 5 | 31% | 21% | 35% |
| Spec 5 | 81% | 82% | 80% |
| Cutoff 6 | 21.9 | 57.1 | 19.7 |
| Sens 6 | 11% | 0% | 13% |
| Spec 6 | 90% | 92% | 91% |
| OR Quart 2 | 2.7 | 2.2 | 1.0 |
| p Value | 0.074 | 0.42 | 1.0 |
| 95% CI of | 0.91 | 0.33 | 0.28 |
| OR Quart2 | 8.0 | 14 | 3.5 |
| OR Quart 3 | 1.9 | 3.0 | 1.2 |
| p Value | 0.27 | 0.24 | 0.76 |
| 95% CI of | 0.61 | 0.48 | 0.36 |
| OR Quart3 | 5.7 | 18 | 4.1 |
| OR Quart 4 | 3.6 | 1.5 | 2.6 |
| p Value | 0.019 | 0.68 | 0.10 |
| 95% CI of | 1.2 | 0.21 | 0.83 |
| OR Quart4 | 10 | 11 | 8.1 |

Immunoglogulin G1

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 4050 | 6810 | 4770 | 9980 | 3950 | 7590 |
| Average | 6150 | 8820 | 8120 | 10500 | 6060 | 7790 |
| Stdev | 6120 | 7880 | 8760 | 8330 | 5490 | 5330 |
| p(t-test) | | 0.023 | | 0.37 | | 0.13 |
| Min | 342 | 572 | 342 | 699 | 424 | 634 |
| Max | 35500 | 30900 | 35500 | 28400 | 27500 | 22200 |
| n (Samp) | 122 | 44 | 48 | 14 | 96 | 30 |
| n (Patient) | 122 | 44 | 48 | 14 | 96 | 30 |

| | At Enrollment | | |
|---|---|---|---|
| | sCr or UO | sCr only | UO only |
| AUC | 0.60 | 0.59 | 0.61 |
| SE | 0.051 | 0.089 | 0.061 |
| p | 0.054 | 0.32 | 0.060 |
| nCohort 1 | 122 | 48 | 96 |
| nCohort 2 | 44 | 14 | 30 |
| Cutoff 1 | 3600 | 3630 | 4450 |
| Sens 1 | 70% | 71% | 70% |
| Spec 1 | 45% | 42% | 57% |
| Cutoff 2 | 1790 | 1480 | 2710 |
| Sens 2 | 82% | 86% | 80% |
| Spec 2 | 20% | 17% | 33% |
| Cutoff 3 | 1470 | 1420 | 1600 |
| Sens 3 | 91% | 93% | 90% |
| Spec 3 | 13% | 17% | 14% |
| Cutoff 4 | 6730 | 6730 | 7270 |
| Sens 4 | 50% | 64% | 53% |
| Spec 4 | 70% | 71% | 71% |
| Cutoff 5 | 9090 | 16800 | 10500 |
| Sens 5 | 36% | 21% | 27% |
| Spec 5 | 80% | 81% | 80% |
| Cutoff 6 | 13500 | 22800 | 13100 |
| Sens 6 | 23% | 7% | 17% |
| Spec 6 | 90% | 92% | 91% |
| OR Quart 2 | 0.62 | 0.18 | 0.43 |
| p Value | 0.39 | 0.15 | 0.27 |
| 95% CI of | 0.21 | 0.018 | 0.098 |
| OR Quart2 | 1.8 | 1.9 | 1.9 |
| OR Quart 3 | 1.1 | 1.0 | 2.3 |
| p Value | 0.80 | 1.0 | 0.16 |
| 95% CI of | 0.42 | 0.20 | 0.72 |
| OR Quart3 | 3.1 | 5.0 | 7.3 |
| OR Quart 4 | 1.9 | 1.2 | 1.9 |
| p Value | 0.18 | 0.78 | 0.28 |
| 95% CI of | 0.74 | 0.26 | 0.59 |
| OR Quart4 | 4.9 | 5.9 | 6.1 |

Interleukin-11

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 215 | 179 | 223 | 200 | 217 | 157 |
| Average | 309 | 246 | 315 | 224 | 310 | 194 |
| Stdev | 297 | 329 | 363 | 163 | 298 | 153 |
| p(t-test) | | 0.24 | | 0.37 | | 0.044 |
| Min | 13.2 | 14.5 | 13.2 | 31.9 | 41.4 | 14.5 |
| Max | 1780 | 2140 | 2140 | 547 | 1780 | 753 |
| n (Samp) | 124 | 44 | 49 | 14 | 97 | 30 |
| n (Patient) | 124 | 44 | 49 | 14 | 97 | 30 |

| | At Enrollment | | |
|---|---|---|---|
| | sCr or UO | sCr only | UO only |
| AUC | 0.41 | 0.45 | 0.37 |
| SE | 0.051 | 0.089 | 0.061 |
| p | 0.087 | 0.61 | 0.037 |
| nCohort 1 | 124 | 49 | 97 |
| nCohort 2 | 44 | 14 | 30 |
| Cutoff 1 | 113 | 113 | 101 |
| Sens 1 | 70% | 71% | 70% |
| Spec 1 | 23% | 33% | 16% |
| Cutoff 2 | 85.8 | 50.4 | 87.1 |
| Sens 2 | 82% | 86% | 80% |
| Spec 2 | 15% | 12% | 13% |
| Cutoff 3 | 42.5 | 41.4 | 42.5 |
| Sens 3 | 91% | 93% | 90% |
| Spec 3 | 5% | 10% | 3% |
| Cutoff 4 | 347 | 390 | 341 |
| Sens 4 | 16% | 21% | 10% |
| Spec 4 | 70% | 71% | 70% |
| Cutoff 5 | 458 | 426 | 469 |
| Sens 5 | 9% | 14% | 3% |
| Spec 5 | 81% | 82% | 80% |
| Cutoff 6 | 613 | 659 | 601 |
| Sens 6 | 5% | 0% | 3% |
| Spec 6 | 90% | 92% | 91% |
| OR Quart 2 | 1.6 | 3.2 | 3.2 |
| p Value | 0.42 | 0.21 | 0.11 |
| 95% CI of | 0.53 | 0.52 | 0.77 |
| OR Quart2 | 4.6 | 20 | 14 |
| OR Quart 3 | 2.5 | 1.6 | 3.2 |
| p Value | 0.083 | 0.63 | 0.11 |
| 95% CI of | 0.89 | 0.23 | 0.77 |
| OR Quart3 | 7.0 | 11 | 14 |
| OR Quart 4 | 2.2 | 2.5 | 5.3 |
| p Value | 0.13 | 0.33 | 0.019 |
| 95% CI of | 0.79 | 0.39 | 1.3 |
| OR Quart4 | 6.4 | 17 | 22 |

Neutrophil collagenase

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 4380 | 7320 | 3490 | 12000 | 5330 | 5620 |
| Average | 13500 | 36800 | 8510 | 15400 | 17400 | 43100 |
| Stdev | 23700 | 118000 | 13200 | 19200 | 27900 | 138000 |
| p(t-test) | | 0.027 | | 0.10 | | 0.066 |
| Min | 150 | 23.8 | 23.8 | 25.8 | 124 | 0.260 |
| Max | 128000 | 670000 | 63000 | 62500 | 128000 | 670000 |
| n (Samp) | 138 | 58 | 56 | 16 | 108 | 42 |
| n (Patient) | 138 | 58 | 56 | 16 | 108 | 42 |

| | At Enrollment | | |
|---|---|---|---|
| | sCr or UO | sCr only | UO only |
| AUC | 0.58 | 0.61 | 0.54 |
| SE | 0.046 | 0.083 | 0.053 |
| p | 0.096 | 0.19 | 0.47 |
| nCohort 1 | 138 | 56 | 108 |
| nCohort 2 | 58 | 16 | 42 |
| Cutoff 1 | 2620 | 1580 | 2940 |
| Sens 1 | 71% | 75% | 71% |
| Spec 1 | 41% | 36% | 39% |
| Cutoff 2 | 1580 | 987 | 1630 |
| Sens 2 | 81% | 81% | 81% |
| Spec 2 | 32% | 25% | 27% |
| Cutoff 3 | 566 | 365 | 1110 |
| Sens 3 | 91% | 94% | 90% |
| Spec 3 | 10% | 11% | 21% |
| Cutoff 4 | 9550 | 7860 | 12900 |
| Sens 4 | 41% | 56% | 38% |
| Spec 4 | 70% | 71% | 70% |
| Cutoff 5 | 15700 | 10700 | 22200 |
| Sens 5 | 33% | 56% | 24% |
| Spec 5 | 80% | 80% | 81% |
| Cutoff 6 | 48400 | 28300 | 60700 |
| Sens 6 | 12% | 12% | 5% |
| Spec 6 | 91% | 91% | 91% |
| OR Quart 2 | 1.9 | 0.70 | 1.9 |
| p Value | 0.17 | 0.67 | 0.23 |
| 95% CI of | 0.76 | 0.13 | 0.67 |
| OR Quart2 | 4.7 | 3.7 | 5.3 |
| OR Quart 3 | 1.4 | 0.44 | 0.85 |
| p Value | 0.48 | 0.38 | 0.77 |
| 95% CI of | 0.55 | 0.069 | 0.27 |
| OR Quart3 | 3.6 | 2.8 | 2.6 |
| OR Quart 4 | 2.5 | 2.2 | 2.1 |
| p Value | 0.049 | 0.28 | 0.15 |
| 95% CI of | 1.0 | 0.52 | 0.76 |
| OR Quart4 | 6.1 | 9.6 | 5.9 |

TABLE 4
Comparison of the maximum marker levels in urine samples collected from Cohort 1 (patients that did not progress beyond RIFLE stage 0) and the
maximum values in urine samples collected from subjects between enrollment and 0, 24 hours, and 48 hours prior to reaching stage F in Cohort 2.
C-C motif chemokine 18

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.531 | 2.90 | 0.531 | 2.72 | 0.531 | 2.38 |
| Average | 2.66 | 12.9 | 2.66 | 10.2 | 2.66 | 9.02 |
| Stdev | 7.47 | 16.2 | 7.47 | 14.4 | 7.47 | 15.4 |
| p (t-test) | | 1.0E−8 | | 8.6E−6 | | 0.0030 |
| Min | 3.13E−5 | 0.226 | 3.13E−5 | 0.204 | 3.13E−5 | 0.126 |
| Max | 40.0 | 40.0 | 40.0 | 40.0 | 40.0 | 40.0 |
| n (Samp) | 223 | 30 | 223 | 30 | 223 | 16 |
| n (Patient) | 223 | 30 | 223 | 30 | 223 | 16 |
| sCr only | | | | | | |
| Median | 0.649 | 2.95 | 0.649 | 2.72 | 0.649 | 2.95 |
| Average | 3.73 | 15.1 | 3.73 | 8.44 | 3.73 | 8.52 |
| Stdev | 9.09 | 18.0 | 9.09 | 11.7 | 9.09 | 13.3 |
| p (t-test) | | 3.0E−5 | | 0.070 | | 0.17 |
| Min | 3.13E−5 | 0.460 | 3.13E−5 | 0.204 | 3.13E−5 | 0.460 |
| Max | 40.0 | 40.0 | 40.0 | 40.0 | 40.0 | 37.8 |
| n (Samp) | 374 | 13 | 374 | 13 | 374 | 7 |
| n (Patient) | 374 | 13 | 374 | 13 | 374 | 7 |
| UO only | | | | | | |
| Median | 0.620 | 2.95 | 0.620 | 2.85 | 0.620 | 2.53 |
| Average | 3.05 | 13.9 | 3.05 | 13.2 | 3.05 | 9.95 |
| Stdev | 7.82 | 16.8 | 7.82 | 16.7 | 7.82 | 16.3 |
| p (t-test) | | 3.6E−7 | | 1.8E−6 | | 0.0048 |
| Min | 3.13E−5 | 0.226 | 3.13E−5 | 0.226 | 3.13E−5 | 0.126 |
| Max | 40.0 | 40.0 | 40.0 | 40.0 | 40.0 | 40.0 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.82 | 0.79 | 0.80 | 0.80 | 0.75 | 0.79 | 0.72 | 0.78 | 0.68 |
| SE | 0.048 | 0.076 | 0.057 | 0.050 | 0.079 | 0.058 | 0.074 | 0.10 | 0.081 |
| p | 2.2E−11 | 1.2E−4 | 1.0E−7 | 2.6E−9 | 0.0014 | 6.2E−7 | 0.0028 | 0.0079 | 0.027 |
| nCohort 1 | 223 | 374 | 173 | 223 | 374 | 173 | 223 | 374 | 173 |
| nCohort 2 | 30 | 13 | 23 | 30 | 13 | 23 | 16 | 7 | 14 |
| Cutoff 1 | 1.98 | 1.97 | 1.98 | 1.98 | 1.97 | 1.98 | 0.714 | 1.99 | 1.34 |
| Sens 1 | 70% | 77% | 74% | 70% | 77% | 74% | 75% | 71% | 71% |
| Spec 1 | 83% | 76% | 76% | 83% | 76% | 76% | 61% | 76% | 71% |
| Cutoff 2 | 1.48 | 1.04 | 1.51 | 1.04 | 1.04 | 1.34 | 0.451 | 1.97 | 0.228 |
| Sens 2 | 80% | 85% | 83% | 80% | 85% | 83% | 81% | 86% | 86% |
| Spec 2 | 78% | 63% | 71% | 72% | 63% | 71% | 45% | 76% | 18% |
| Cutoff 3 | 0.531 | 0.531 | 0.862 | 0.451 | 0.451 | 0.767 | 0.225 | 0.451 | 0.205 |
| Sens 3 | 90% | 92% | 91% | 90% | 92% | 91% | 94% | 100% | 93% |
| Spec 3 | 50% | 43% | 60% | 45% | 39% | 57% | 28% | 39% | 18% |
| Cutoff 4 | 1.03 | 1.41 | 1.34 | 1.03 | 1.41 | 1.34 | 1.03 | 1.41 | 1.34 |
| Sens 4 | 83% | 77% | 87% | 80% | 77% | 83% | 69% | 86% | 71% |
| Spec 4 | 70% | 70% | 71% | 70% | 70% | 71% | 70% | 70% | 71% |
| Cutoff 5 | 1.71 | 2.82 | 2.68 | 1.71 | 2.82 | 2.68 | 1.71 | 2.82 | 2.68 |
| Sens 5 | 73% | 54% | 61% | 73% | 46% | 57% | 62% | 57% | 50% |
| Spec 5 | 80% | 80% | 80% | 80% | 80% | 80% | 80% | 80% | 80% |
| Cutoff 6 | 3.97 | 8.25 | 4.19 | 3.97 | 8.25 | 4.19 | 3.97 | 8.25 | 4.19 |
| Sens 6 | 43% | 38% | 43% | 37% | 31% | 39% | 25% | 29% | 21% |
| Spec 6 | 90% | 90% | 90% | 90% | 90% | 90% | 90% | 90% | 90% |
| OR Quart 2 | 3.1 | >2.0 | 0 | 3.1 | 0.99 | 0 | 3.1 | >1.0 | 0 |
| p Value | 0.33 | <0.57 | na | 0.33 | 0.99 | na | 0.34 | <0.99 | na |
| 95% CI of | 0.31 | >0.18 | na | 0.31 | 0.061 | na | 0.31 | >0.062 | na |
| OR Quart2 | 31 | na | na | 31 | 16 | na | 30 | na | na |
| OR Quart 3 | 4.2 | >3.1 | 3.9 | 4.2 | 3.0 | 3.9 | 0.98 | >0 | 0.98 |
| p Value | 0.20 | <0.34 | 0.100 | 0.20 | 0.34 | 0.100 | 0.99 | <na | 0.98 |
| 95% CI of | 0.46 | >0.31 | 0.77 | 0.46 | 0.31 | 0.77 | 0.060 | >na | 0.19 |
| OR Quart3 | 39 | na | 20 | 39 | 30 | 20 | 16 | na | 5.1 |
| OR Quart 4 | 32 | >8.6 | 9.4 | 32 | 8.5 | 9.4 | 13 | >6.3 | 2.9 |
| p Value | 8.4E−4 | <0.044 | 0.0045 | 8.4E−4 | 0.045 | 0.0045 | 0.016 | <0.090 | 0.13 |
| 95% CI of | 4.2 | >1.1 | 2.0 | 4.2 | 1.0 | 2.0 | 1.6 | >0.75 | 0.73 |
| OR Quart4 | 250 | na | 44 | 250 | 70 | 44 | 100 | na | 12 |

C-C motif chemokine 24

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 13.0 | 51.0 | 13.0 | 30.9 | 13.0 | 26.4 |
| Average | 26.0 | 308 | 26.0 | 269 | 26.0 | 95.6 |
| Stdev | 58.4 | 690 | 58.4 | 598 | 58.4 | 288 |
| p (t-test) | | 5.8E−9 | | 1.0E−8 | | 0.0037 |
| Min | 0.0120 | 2.55 | 0.0120 | 2.55 | 0.0120 | 2.55 |
| Max | 730 | 2790 | 730 | 2380 | 730 | 1170 |
| n (Samp) | 223 | 30 | 223 | 30 | 223 | 16 |
| n (Patient) | 223 | 30 | 223 | 30 | 223 | 16 |
| sCr only | | | | | | |
| Median | 17.8 | 30.9 | 17.8 | 28.7 | 17.8 | 28.0 |
| Average | 48.4 | 93.4 | 48.4 | 63.7 | 48.4 | 53.5 |
| Stdev | 160 | 110 | 160 | 74.6 | 160 | 86.7 |
| p (t-test) | | 0.32 | | 0.73 | | 0.93 |
| Min | 0.0120 | 2.55 | 0.0120 | 2.55 | 0.0120 | 2.55 |
| Max | 2380 | 373 | 2380 | 249 | 2380 | 249 |
| n (Samp) | 375 | 13 | 375 | 13 | 375 | 7 |
| n (Patient) | 375 | 13 | 375 | 13 | 375 | 7 |
| UO only | | | | | | |
| Median | 13.9 | 61.9 | 13.9 | 41.6 | 13.9 | 28.3 |
| Average | 30.6 | 390 | 30.6 | 342 | 30.6 | 112 |
| Stdev | 66.3 | 773 | 66.3 | 669 | 66.3 | 306 |
| p (t-test) | | 7.2E−9 | | 9.0E−9 | | 0.0051 |
| Min | 0.0120 | 3.62 | 0.0120 | 3.62 | 0.0120 | 3.62 |
| Max | 730 | 2790 | 730 | 2380 | 730 | 1170 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.81 | 0.72 | 0.83 | 0.78 | 0.66 | 0.81 | 0.66 | 0.58 | 0.70 |
| SE | 0.049 | 0.082 | 0.054 | 0.051 | 0.084 | 0.056 | 0.076 | 0.11 | 0.081 |
| p | 2.2E−10 | 0.0078 | 6.4E−10 | 4.2E−8 | 0.051 | 2.2E−8 | 0.034 | 0.49 | 0.015 |
| nCohort 1 | 223 | 375 | 173 | 223 | 375 | 173 | 223 | 375 | 173 |
| nCohort 2 | 30 | 13 | 23 | 30 | 13 | 23 | 16 | 7 | 14 |
| Cutoff 1 | 28.4 | 27.3 | 30.4 | 27.2 | 25.9 | 27.8 | 16.6 | 26.9 | 20.4 |
| Sens 1 | 70% | 77% | 74% | 70% | 77% | 74% | 75% | 71% | 71% |
| Spec 1 | 78% | 64% | 77% | 76% | 62% | 75% | 61% | 63% | 66% |
| Cutoff 2 | 25.4 | 25.4 | 27.8 | 20.4 | 10.9 | 26.6 | 8.65 | 8.65 | 16.6 |
| Sens 2 | 80% | 85% | 83% | 80% | 85% | 83% | 81% | 86% | 86% |
| Spec 2 | 73% | 61% | 75% | 68% | 35% | 73% | 40% | 31% | 58% |
| Cutoff 3 | 16.6 | 8.65 | 19.3 | 10.9 | 8.65 | 19.3 | 3.21 | 2.51 | 3.71 |
| Sens 3 | 90% | 92% | 91% | 90% | 92% | 91% | 94% | 100% | 93% |
| Spec 3 | 61% | 31% | 63% | 48% | 31% | 63% | 28% | 18% | 25% |
| Cutoff 4 | 23.3 | 36.9 | 24.6 | 23.3 | 36.9 | 24.6 | 23.3 | 36.9 | 24.6 |
| Sens 4 | 80% | 46% | 83% | 77% | 38% | 83% | 56% | 14% | 64% |
| Spec 4 | 71% | 70% | 71% | 71% | 70% | 71% | 71% | 70% | 71% |
| Cutoff 5 | 35.1 | 51.0 | 40.3 | 35.1 | 51.0 | 40.3 | 35.1 | 51.0 | 40.3 |
| Sens 5 | 53% | 46% | 61% | 43% | 38% | 52% | 19% | 14% | 29% |
| Spec 5 | 80% | 80% | 80% | 80% | 80% | 80% | 80% | 80% | 80% |
| Cutoff 6 | 62.8 | 87.2 | 74.4 | 62.8 | 87.2 | 74.4 | 62.8 | 87.2 | 74.4 |
| Sens 6 | 43% | 31% | 48% | 37% | 15% | 39% | 6% | 14% | 14% |
| Spec 6 | 90% | 90% | 90% | 90% | 90% | 90% | 90% | 90% | 90% |
| OR Quart 2 | 2.0 | 1.0 | 0 | 3.1 | 2.0 | 0 | 3.1 | 0.99 | 0 |
| p Value | 0.57 | 1.0 | na | 0.33 | 0.57 | na | 0.34 | 0.99 | na |
| 95% CI of | 0.18 | 0.062 | na | 0.31 | 0.18 | na | 0.31 | 0.061 | na |
| OR Quart2 | 23 | 16 | na | 31 | 23 | na | 30 | 16 | na |
| OR Quart 3 | 12 | 5.2 | 9.4 | 12 | 5.2 | 12 | 4.1 | 4.1 | 3.2 |
| p Value | 0.021 | 0.13 | 0.039 | 0.021 | 0.13 | 0.019 | 0.21 | 0.21 | 0.17 |
| 95% CI of | 1.4 | 0.60 | 1.1 | 1.4 | 0.60 | 1.5 | 0.45 | 0.45 | 0.61 |
| OR Quart3 | 94 | 46 | 78 | 94 | 46 | 100 | 38 | 38 | 17 |
| OR Quart 4 | 22 | 6.3 | 19 | 21 | 5.2 | 16 | 8.9 | 0.99 | 3.2 |
| p Value | 0.0030 | 0.090 | 0.0053 | 0.0039 | 0.13 | 0.0098 | 0.042 | 0.99 | 0.17 |
| 95% CI of | 2.9 | 0.75 | 2.4 | 2.6 | 0.60 | 1.9 | 1.1 | 0.061 | 0.61 |
| OR Quart4 | 170 | 54 | 150 | 160 | 46 | 130 | 74 | 16 | 17 |

C-C motif chemokine 8

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 2.74 | 24.0 | 2.74 | 22.9 | 2.74 | 13.6 |
| Average | 11.9 | 175 | 11.9 | 169 | 11.9 | 250 |
| Stdev | 27.9 | 664 | 27.9 | 665 | 27.9 | 912 |
| p (t-test) | | 2.8E−4 | | 4.6E−4 | | 9.2E−5 |
| Min | 0.0250 | 0.206 | 0.0250 | 0.136 | 0.0250 | 0.136 |
| Max | 250 | 3670 | 250 | 3670 | 250 | 3670 |
| n (Samp) | 223 | 30 | 223 | 30 | 223 | 16 |
| n (Patient) | 223 | 30 | 223 | 30 | 223 | 16 |
| sCr only | | | | | | |
| Median | 4.72 | 22.7 | 4.72 | 22.7 | 4.72 | 22.7 |
| Average | 17.7 | 49.4 | 17.7 | 45.4 | 17.7 | 36.6 |
| Stdev | 47.6 | 82.2 | 47.6 | 79.2 | 47.6 | 47.0 |
| p (t-test) | | 0.022 | | 0.045 | | 0.30 |
| Min | 0.0250 | 0.206 | 0.0250 | 0.206 | 0.0250 | 4.53 |
| Max | 492 | 279 | 492 | 279 | 492 | 140 |
| n (Samp) | 375 | 13 | 375 | 13 | 375 | 7 |
| n (Patient) | 375 | 13 | 375 | 13 | 375 | 7 |
| UO only | | | | | | |
| Median | 4.72 | 30.8 | 4.72 | 30.8 | 4.72 | 13.6 |
| Average | 14.8 | 220 | 14.8 | 209 | 14.8 | 284 |
| Stdev | 32.0 | 755 | 32.0 | 757 | 32.0 | 975 |
| p (t-test) | | 3.9E−4 | | 7.8E−4 | | 2.7E−4 |
| Min | 0.0250 | 0.829 | 0.0250 | 0.136 | 0.0250 | 0.136 |
| Max | 250 | 3670 | 250 | 3670 | 250 | 3670 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.79 | 0.70 | 0.82 | 0.77 | 0.69 | 0.78 | 0.70 | 0.77 | 0.65 |
| SE | 0.051 | 0.083 | 0.056 | 0.052 | 0.083 | 0.059 | 0.075 | 0.11 | 0.082 |
| p | 6.8E−9 | 0.017 | 1.3E−8 | 3.2E−7 | 0.022 | 2.1E−6 | 0.0080 | 0.010 | 0.078 |
| nCohort 1 | 223 | 375 | 173 | 223 | 375 | 173 | 223 | 375 | 173 |
| nCohort 2 | 30 | 13 | 23 | 30 | 13 | 23 | 16 | 7 | 14 |
| Cutoff 1 | 10.4 | 4.68 | 12.5 | 9.39 | 4.35 | 10.4 | 4.35 | 21.7 | 4.35 |
| Sens 1 | 70% | 77% | 74% | 70% | 77% | 74% | 75% | 71% | 71% |
| Spec 1 | 74% | 48% | 73% | 71% | 48% | 69% | 56% | 81% | 47% |
| Cutoff 2 | 7.37 | 4.35 | 9.05 | 4.35 | 3.16 | 8.05 | 1.21 | 7.37 | 0.756 |
| Sens 2 | 80% | 85% | 83% | 80% | 85% | 83% | 81% | 86% | 86% |
| Spec 2 | 67% | 48% | 66% | 56% | 46% | 65% | 39% | 59% | 26% |
| Cutoff 3 | 1.21 | 0.438 | 4.35 | 0.650 | 0.438 | 1.21 | 0.438 | 4.35 | 0.438 |
| Sens 3 | 90% | 92% | 91% | 90% | 92% | 91% | 94% | 100% | 93% |
| Spec 3 | 39% | 24% | 47% | 32% | 24% | 31% | 30% | 48% | 21% |
| Cutoff 4 | 9.38 | 12.0 | 11.1 | 9.38 | 12.0 | 11.1 | 9.38 | 12.0 | 11.1 |
| Sens 4 | 73% | 62% | 74% | 70% | 62% | 70% | 56% | 71% | 57% |
| Spec 4 | 70% | 70% | 71% | 70% | 70% | 71% | 70% | 70% | 71% |
| Cutoff 5 | 12.8 | 20.3 | 18.0 | 12.8 | 20.3 | 18.0 | 12.8 | 20.3 | 18.0 |
| Sens 5 | 63% | 62% | 70% | 63% | 62% | 65% | 50% | 71% | 43% |
| Spec 5 | 80% | 80% | 80% | 80% | 80% | 80% | 80% | 80% | 80% |
| Cutoff 6 | 31.5 | 36.6 | 34.7 | 31.5 | 36.6 | 34.7 | 31.5 | 36.6 | 34.7 |
| Sens 6 | 37% | 23% | 48% | 37% | 15% | 48% | 19% | 14% | 21% |
| Spec 6 | 90% | 90% | 90% | 90% | 90% | 90% | 90% | 90% | 90% |
| OR Quart 2 | 3.1 | 0.49 | 2.0 | 2.1 | 1.0 | 1.0 | 3.1 | >1.0 | 1.5 |
| p Value | 0.33 | 0.57 | 0.57 | 0.41 | 1.0 | 1.0 | 0.34 | <1.0 | 0.67 |
| 95% CI of | 0.31 | 0.044 | 0.18 | 0.36 | 0.14 | 0.14 | 0.31 | >0.062 | 0.24 |
| OR Quart2 | 31 | 5.5 | 23 | 12 | 7.2 | 7.4 | 30 | na | 9.4 |
| OR Quart 3 | 7.8 | 1.0 | 4.3 | 2.6 | 0.49 | 2.1 | 4.1 | >1.0 | 1.5 |
| p Value | 0.059 | 1.0 | 0.20 | 0.26 | 0.57 | 0.41 | 0.21 | <0.99 | 0.67 |
| 95% CI of | 0.92 | 0.14 | 0.46 | 0.49 | 0.044 | 0.36 | 0.45 | >0.062 | 0.24 |
| OR Quart3 | 65 | 7.2 | 40 | 14 | 5.5 | 12 | 38 | na | 9.4 |
| OR Quart 4 | 26 | 4.3 | 23 | 13 | 4.3 | 10 | 8.9 | >5.2 | 3.2 |
| p Value | 0.0018 | 0.071 | 0.0029 | 8.9E−4 | 0.071 | 0.0029 | 0.042 | <0.13 | 0.17 |
| 95% CI of | 3.4 | 0.88 | 2.9 | 2.9 | 0.88 | 2.2 | 1.1 | >0.60 | 0.61 |
| OR Quart4 | 200 | 21 | 180 | 58 | 21 | 48 | 74 | na | 17 |

Cathepsin D

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 71300 | 128000 | 71300 | 123000 | 71300 | 121000 |
| Average | 74100 | 136000 | 74100 | 131000 | 74100 | 130000 |
| Stdev | 38900 | 41600 | 38900 | 39100 | 38900 | 42500 |
| p (t-test) | | 1.8E−14 | | 7.3E−13 | | 7.9E−8 |
| Min | 656 | 65600 | 656 | 65600 | 656 | 65600 |
| Max | 200000 | 200000 | 200000 | 200000 | 200000 | 200000 |
| n (Samp) | 223 | 30 | 223 | 30 | 223 | 16 |
| n (Patient) | 223 | 30 | 223 | 30 | 223 | 16 |
| sCr only | | | | | | |
| Median | 84600 | 130000 | 84600 | 120000 | 84600 | 117000 |
| Average | 90900 | 127000 | 90900 | 121000 | 90900 | 127000 |
| Stdev | 44700 | 34200 | 44700 | 29100 | 44700 | 24800 |
| p (t-test) | | 0.0040 | | 0.018 | | 0.035 |
| Min | 656 | 71400 | 656 | 71400 | 656 | 110000 |
| Max | 200000 | 188000 | 200000 | 181000 | 200000 | 181000 |
| n (Samp) | 374 | 13 | 374 | 13 | 374 | 7 |
| n (Patient) | 374 | 13 | 374 | 13 | 374 | 7 |
| UO only | | | | | | |
| Median | 78900 | 138000 | 78900 | 130000 | 78900 | 125000 |
| Average | 84300 | 143000 | 84300 | 138000 | 84300 | 129000 |
| Stdev | 40600 | 41200 | 40600 | 40000 | 40600 | 43300 |
| p (t-test) | | 7.6E−10 | | 9.9E−9 | | 1.0E−4 |
| Min | 2520 | 65600 | 2520 | 65600 | 2520 | 65600 |
| Max | 200000 | 200000 | 200000 | 200000 | 200000 | 200000 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.86 | 0.74 | 0.84 | 0.85 | 0.72 | 0.83 | 0.84 | 0.77 | 0.78 |
| SE | 0.044 | 0.080 | 0.053 | 0.045 | 0.081 | 0.054 | 0.063 | 0.11 | 0.075 |
| p | 4.4E−16 | 0.0021 | 9.5E−11 | 1.2E−14 | 0.0069 | 1.7E−9 | 9.2E−8 | 0.011 | 1.9E−4 |
| nCohort 1 | 223 | 374 | 173 | 223 | 374 | 173 | 223 | 374 | 173 |
| nCohort 2 | 30 | 13 | 23 | 30 | 13 | 23 | 16 | 7 | 14 |
| Cutoff 1 | 117000 | 109000 | 119000 | 112000 | 109000 | 112000 | 109000 | 115000 | 109000 |
| Sens 1 | 70% | 77% | 74% | 70% | 77% | 74% | 75% | 71% | 71% |
| Spec 1 | 86% | 70% | 81% | 84% | 70% | 77% | 83% | 72% | 74% |
| Cutoff 2 | 109000 | 92900 | 111000 | 109000 | 89500 | 110000 | 99400 | 112000 | 96100 |
| Sens 2 | 80% | 85% | 83% | 80% | 85% | 83% | 81% | 86% | 86% |
| Spec 2 | 83% | 56% | 75% | 83% | 53% | 75% | 76% | 72% | 65% |
| Cutoff 3 | 84400 | 84400 | 96100 | 84400 | 84400 | 96100 | 66900 | 109000 | 66900 |
| Sens 3 | 90% | 92% | 91% | 90% | 92% | 91% | 94% | 100% | 93% |
| Spec 3 | 67% | 50% | 65% | 67% | 50% | 65% | 48% | 70% | 39% |
| Cutoff 4 | 92600 | 110000 | 103000 | 92600 | 110000 | 103000 | 92600 | 110000 | 103000 |
| Sens 4 | 87% | 69% | 87% | 83% | 69% | 87% | 88% | 86% | 71% |
| Spec 4 | 70% | 70% | 71% | 70% | 70% | 71% | 70% | 70% | 71% |
| Cutoff 5 | 104000 | 133000 | 117000 | 104000 | 133000 | 117000 | 104000 | 133000 | 117000 |
| Sens 5 | 80% | 38% | 78% | 80% | 23% | 70% | 75% | 14% | 57% |
| Spec 5 | 80% | 80% | 80% | 80% | 80% | 80% | 80% | 80% | 80% |
| Cutoff 6 | 133000 | 151000 | 143000 | 133000 | 151000 | 143000 | 133000 | 151000 | 143000 |
| Sens 6 | 43% | 15% | 43% | 40% | 8% | 39% | 38% | 14% | 36% |
| Spec 6 | 90% | 90% | 90% | 90% | 90% | 90% | 90% | 90% | 90% |
| OR Quart 2 | >3.1 | >2.0 | >2.1 | >3.1 | >2.0 | >2.1 | >2.0 | >0 | >2.0 |
| p Value | <0.33 | <0.57 | <0.55 | <0.33 | <0.57 | <0.55 | <0.57 | <na | <0.56 |
| 95% CI of | >0.32 | >0.18 | >0.18 | >0.32 | >0.18 | >0.18 | >0.18 | >na | >0.18 |
| OR Quart2 | na | na | na | na | na | na | na | na | na |
| OR Quart 3 | >3.1 | >4.1 | >5.6 | >3.1 | >5.2 | >6.8 | >2.0 | >5.3 | >4.3 |
| p Value | <0.33 | <0.21 | <0.12 | <0.33 | <0.13 | <0.081 | <0.57 | <0.13 | <0.20 |
| 95% CI of | >0.32 | >0.45 | >0.63 | >0.32 | >0.60 | >0.79 | >0.18 | >0.60 | >0.46 |
| OR Quart3 | na | na | na | na | na | na | na | na | na |
| OR Quart 4 | >38 | >7.5 | >24 | >38 | >6.3 | >22 | >15 | >2.0 | >9.4 |
| p Value | <4.8E−4 | <0.062 | <0.0027 | <4.8E−4 | <0.090 | <0.0036 | <0.011 | <0.57 | <0.038 |
| 95% CI of | >4.9 | >0.90 | >3.0 | >4.9 | >0.75 | >2.7 | >1.9 | >0.18 | >1.1 |
| OR Quart4 | na | na | na | na | na | na | na | na | na |

C—X—C motif chemokine 13

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.224 | 9.29 | 0.224 | 8.44 | 0.224 | 3.11 |
| Average | 10.4 | 107 | 10.4 | 104 | 10.4 | 39.9 |
| Stdev | 61.2 | 355 | 61.2 | 341 | 61.2 | 109 |
| p (t-test) | | 2.4E−4 | | 2.5E−4 | | 0.082 |
| Min | 0.00269 | 0.0163 | 0.00269 | 0.0163 | 0.00269 | 0.0163 |
| Max | 832 | 1930 | 832 | 1850 | 832 | 440 |
| n (Samp) | 223 | 30 | 223 | 30 | 223 | 16 |
| n (Patient) | 223 | 30 | 223 | 30 | 223 | 16 |
| sCr only | | | | | | |
| Median | 0.772 | 8.40 | 0.772 | 5.14 | 0.772 | 5.14 |
| Average | 11.8 | 18.6 | 11.8 | 16.7 | 11.8 | 15.2 |
| Stdev | 53.0 | 27.8 | 53.0 | 27.8 | 53.0 | 28.4 |
| p (t-test) | | 0.65 | | 0.74 | | 0.87 |
| Min | 0.00269 | 0.0174 | 0.00269 | 0.0174 | 0.00269 | 0.0222 |
| Max | 832 | 80.5 | 832 | 79.0 | 832 | 79.0 |
| n (Samp) | 375 | 13 | 375 | 13 | 375 | 7 |
| n (Patient) | 375 | 13 | 375 | 13 | 375 | 7 |
| UO only | | | | | | |
| Median | 0.392 | 23.5 | 0.392 | 20.4 | 0.392 | 2.87 |
| Average | 12.0 | 139 | 12.0 | 135 | 12.0 | 45.2 |
| Stdev | 68.6 | 402 | 68.6 | 387 | 68.6 | 117 |
| p (t-test) | | 1.9E−4 | | 1.9E−4 | | 0.10 |
| Min | 0.00269 | 0.0163 | 0.00269 | 0.0163 | 0.00269 | 0.0163 |
| Max | 832 | 1930 | 832 | 1850 | 832 | 440 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.82 | 0.73 | 0.83 | 0.81 | 0.71 | 0.82 | 0.73 | 0.70 | 0.72 |
| SE | 0.048 | 0.081 | 0.054 | 0.049 | 0.082 | 0.055 | 0.073 | 0.11 | 0.079 |
| p | 5.8E−11 | 0.0040 | 1.5E−9 | 4.8E−10 | 0.011 | 3.4E−9 | 0.0014 | 0.078 | 0.0048 |
| nCohort 1 | 223 | 375 | 173 | 223 | 375 | 173 | 223 | 375 | 173 |
| nCohort 2 | 30 | 13 | 23 | 30 | 13 | 23 | 16 | 7 | 14 |
| Cutoff 1 | 4.27 | 2.65 | 2.84 | 2.84 | 2.57 | 2.84 | 1.18 | 3.20 | 2.32 |
| Sens 1 | 70% | 77% | 74% | 70% | 77% | 74% | 75% | 71% | 71% |
| Spec 1 | 80% | 67% | 71% | 74% | 66% | 71% | 65% | 70% | 70% |
| Cutoff 2 | 2.65 | 0.449 | 2.65 | 2.49 | 0.449 | 2.65 | 0.448 | 0.449 | 0.448 |
| Sens 2 | 80% | 85% | 83% | 80% | 85% | 83% | 81% | 86% | 86% |
| Spec 2 | 74% | 45% | 71% | 73% | 45% | 71% | 56% | 45% | 51% |
| Cutoff 3 | 0.0193 | 0.0193 | 0.448 | 0.0193 | 0.0193 | 0.448 | 0.0155 | 0.0193 | 0.0155 |
| Sens 3 | 90% | 92% | 91% | 90% | 92% | 91% | 100% | 100% | 100% |
| Spec 3 | 42% | 32% | 51% | 42% | 32% | 51% | 39% | 32% | 31% |
| Cutoff 4 | 1.82 | 3.20 | 2.49 | 1.82 | 3.20 | 2.49 | 1.82 | 3.20 | 2.49 |
| Sens 4 | 80% | 69% | 83% | 80% | 62% | 83% | 69% | 71% | 64% |
| Spec 4 | 70% | 70% | 71% | 70% | 70% | 71% | 70% | 70% | 71% |
| Cutoff 5 | 4.64 | 7.41 | 5.36 | 4.64 | 7.41 | 5.36 | 4.64 | 7.41 | 5.36 |
| Sens 5 | 67% | 54% | 65% | 60% | 46% | 65% | 38% | 43% | 36% |
| Spec 5 | 80% | 80% | 80% | 80% | 80% | 80% | 80% | 80% | 80% |
| Cutoff 6 | 12.3 | 18.7 | 14.4 | 12.3 | 18.7 | 14.4 | 12.3 | 18.7 | 14.4 |
| Sens 6 | 47% | 31% | 57% | 43% | 23% | 52% | 25% | 14% | 29% |
| Spec 6 | 90% | 90% | 90% | 90% | 90% | 90% | 90% | 90% | 90% |
| OR Quart 2 | >4.3 | >3.1 | >3.2 | >4.3 | >3.1 | >3.2 | >3.1 | >2.0 | >3.1 |
| p Value | <0.20 | <0.33 | <0.32 | <0.20 | <0.33 | <0.32 | <0.33 | <0.57 | <0.33 |
| 95% CI of | >0.46 | >0.32 | >0.32 | >0.46 | >0.32 | >0.32 | >0.31 | >0.18 | >0.31 |
| OR Quart2 | na | na | na | na | na | na | na | na | na |
| OR Quart 3 | >6.6 | >1.0 | >5.6 | >7.9 | >3.1 | >5.6 | >6.6 | >1.0 | >5.5 |
| p Value | <0.084 | <0.99 | <0.12 | <0.057 | <0.33 | <0.12 | <0.086 | <0.99 | <0.13 |
| 95% CI of | >0.77 | >0.062 | >0.63 | >0.94 | >0.32 | >0.63 | >0.76 | >0.062 | >0.61 |
| OR Quart3 | na | na | na | na | na | na | na | na | na |
| OR Quart 4 | >29 | >9.9 | >22 | >27 | >7.5 | >22 | >7.8 | >4.1 | >6.7 |
| p Value | <0.0013 | <0.031 | <0.0036 | <0.0017 | <0.061 | <0.0036 | <0.059 | <0.21 | <0.083 |
| 95% CI of | >3.7 | >1.2 | >2.7 | >3.4 | >0.91 | >2.7 | >0.93 | >0.45 | >0.78 |
| OR Quart4 | na | na | na | na | na | na | na | na | na |

Insulin-like growth factor-binding protein 3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 375 | 686 | 375 | 686 | 375 | 609 |
| Average | 692 | 2080 | 692 | 1580 | 692 | 840 |
| Stdev | 1130 | 3120 | 1130 | 2400 | 1130 | 578 |
| p (t-test) | | 1.1E−6 | | 3.9E−4 | | 0.55 |
| Min | 3.84 | 108 | 3.84 | 88.0 | 3.84 | 186 |
| Max | 12500 | 12500 | 12500 | 12500 | 12500 | 2060 |
| n (Samp) | 235 | 35 | 235 | 35 | 235 | 21 |
| n (Patient) | 235 | 35 | 235 | 35 | 235 | 21 |
| sCr only | | | | | | |
| Median | 574 | 862 | 574 | 862 | 574 | 1000 |
| Average | 949 | 1440 | 949 | 1370 | 949 | 1640 |
| Stdev | 1480 | 1900 | 1480 | 1860 | 1480 | 2210 |
| p (t-test) | | 0.19 | | 0.26 | | 0.13 |
| Min | 3.84 | 108 | 3.84 | 88.0 | 3.84 | 411 |
| Max | 12500 | 8160 | 12500 | 8160 | 12500 | 8160 |
| n (Samp) | 397 | 17 | 397 | 17 | 397 | 11 |
| n (Patient) | 397 | 17 | 397 | 17 | 397 | 11 |
| UO only | | | | | | |
| Median | 434 | 993 | 434 | 993 | 434 | 533 |
| Average | 846 | 2640 | 846 | 1930 | 846 | 792 |
| Stdev | 1490 | 3590 | 1490 | 2790 | 1490 | 631 |
| p (t-test) | | 9.6E−6 | | 0.0028 | | 0.89 |
| Min | 3.84 | 186 | 3.84 | 186 | 3.84 | 186 |
| Max | 12500 | 12500 | 12500 | 12500 | 12500 | 2060 |
| n (Samp) | 190 | 25 | 190 | 25 | 190 | 16 |
| n (Patient) | 190 | 25 | 190 | 25 | 190 | 16 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.70 | 0.62 | 0.70 | 0.69 | 0.61 | 0.69 | 0.66 | 0.68 | 0.58 |
| SE | 0.052 | 0.074 | 0.061 | 0.052 | 0.074 | 0.062 | 0.067 | 0.090 | 0.077 |
| p | 7.3E−5 | 0.11 | 0.0011 | 2.0E−4 | 0.14 | 0.0025 | 0.019 | 0.049 | 0.33 |
| nCohort 1 | 235 | 397 | 190 | 235 | 397 | 190 | 235 | 397 | 190 |
| nCohort 2 | 35 | 17 | 25 | 35 | 17 | 25 | 21 | 11 | 16 |
| Cutoff 1 | 478 | 543 | 478 | 452 | 543 | 452 | 452 | 662 | 293 |
| Sens 1 | 71% | 71% | 72% | 71% | 71% | 72% | 71% | 73% | 75% |
| Spec 1 | 58% | 49% | 54% | 57% | 49% | 53% | 57% | 55% | 36% |
| Cutoff 2 | 388 | 411 | 388 | 323 | 411 | 323 | 323 | 543 | 267 |
| Sens 2 | 80% | 82% | 80% | 80% | 82% | 80% | 81% | 82% | 81% |
| Spec 2 | 51% | 40% | 46% | 44% | 40% | 38% | 44% | 49% | 33% |
| Cutoff 3 | 258 | 108 | 267 | 238 | 108 | 257 | 258 | 456 | 193 |
| Sens 3 | 91% | 94% | 92% | 91% | 94% | 92% | 90% | 91% | 94% |
| Spec 3 | 37% | 14% | 33% | 34% | 14% | 31% | 37% | 44% | 24% |
| Cutoff 4 | 736 | 1060 | 836 | 736 | 1060 | 836 | 736 | 1060 | 836 |
| Sens 4 | 49% | 41% | 52% | 49% | 41% | 52% | 43% | 45% | 38% |
| Spec 4 | 70% | 70% | 70% | 70% | 70% | 70% | 70% | 70% | 70% |
| Cutoff 5 | 1100 | 1380 | 1130 | 1100 | 1380 | 1130 | 1100 | 1380 | 1130 |
| Sens 5 | 40% | 35% | 44% | 40% | 29% | 44% | 29% | 27% | 25% |
| Spec 5 | 80% | 80% | 80% | 80% | 80% | 80% | 80% | 80% | 80% |
| Cutoff 6 | 1500 | 1870 | 1560 | 1500 | 1870 | 1560 | 1500 | 1870 | 1560 |
| Sens 6 | 34% | 24% | 40% | 34% | 18% | 40% | 24% | 18% | 12% |
| Spec 6 | 90% | 90% | 90% | 90% | 90% | 90% | 90% | 90% | 90% |
| OR Quart 2 | 3.7 | 2.0 | 5.3 | 3.7 | 2.0 | 2.6 | >5.4 | >3.1 | 2.0 |
| p Value | 0.11 | 0.42 | 0.13 | 0.11 | 0.42 | 0.27 | <0.13 | <0.33 | 0.42 |
| 95% CI of | 0.75 | 0.36 | 0.60 | 0.75 | 0.36 | 0.48 | >0.62 | >0.32 | 0.36 |
| OR Quart2 | 19 | 11 | 47 | 19 | 11 | 14 | na | na | 12 |
| OR Quart 3 | 7.1 | 2.0 | 9.0 | 7.1 | 2.0 | 3.8 | >9.1 | >4.2 | 3.3 |
| p Value | 0.013 | 0.42 | 0.041 | 0.013 | 0.42 | 0.11 | <0.040 | <0.21 | 0.16 |
| 95% CI of | 1.5 | 0.37 | 1.1 | 1.5 | 0.37 | 0.75 | >1.1 | >0.46 | 0.63 |
| OR Quart3 | 33 | 11 | 75 | 33 | 11 | 19 | na | na | 17 |
| OR Quart 4 | 8.4 | 3.6 | 13 | 8.4 | 3.6 | 6.5 | >9.1 | >4.2 | 2.0 |
| p Value | 0.0062 | 0.11 | 0.015 | 0.0062 | 0.11 | 0.018 | <0.040 | <0.21 | 0.42 |
| 95% CI of | 1.8 | 0.74 | 1.7 | 1.8 | 0.74 | 1.4 | >1.1 | >0.46 | 0.36 |
| OR Quart4 | 39 | 18 | 110 | 39 | 18 | 31 | na | na | 12 |

Immunoglogulin G1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3510 | 14800 | 3510 | 14600 | 3510 | 9360 |
| Average | 6360 | 16700 | 6360 | 15900 | 6360 | 11200 |
| Stdev | 8760 | 14300 | 8760 | 14100 | 8760 | 6710 |
| p (t-test) | | 6.8E−8 | | 5.0E−7 | | 0.032 |
| Min | 59.7 | 980 | 59.7 | 980 | 59.7 | 2380 |
| Max | 80000 | 80000 | 80000 | 80000 | 80000 | 25300 |
| n (Samp) | 222 | 30 | 222 | 30 | 222 | 16 |
| n (Patient) | 222 | 30 | 222 | 30 | 222 | 16 |
| sCr only | | | | | | |
| Median | 4660 | 11200 | 4660 | 11200 | 4660 | 9840 |
| Average | 7920 | 13300 | 7920 | 12300 | 7920 | 10200 |
| Stdev | 9560 | 8270 | 9560 | 7830 | 9560 | 4610 |
| p (t-test) | | 0.046 | | 0.099 | | 0.53 |
| Min | 59.7 | 980 | 59.7 | 980 | 59.7 | 2880 |
| Max | 80000 | 28300 | 80000 | 28300 | 80000 | 15300 |
| n (Samp) | 373 | 13 | 373 | 13 | 373 | 7 |
| n (Patient) | 373 | 13 | 373 | 13 | 373 | 7 |
| UO only | | | | | | |
| Median | 3840 | 14900 | 3840 | 14800 | 3840 | 10900 |
| Average | 6750 | 18700 | 6750 | 18000 | 6750 | 11800 |
| Stdev | 9200 | 15600 | 9200 | 15300 | 9200 | 7010 |
| p (t-test) | | 2.8E−7 | | 1.2E−6 | | 0.046 |
| Min | 59.7 | 2880 | 59.7 | 2880 | 59.7 | 2380 |
| Max | 80000 | 80000 | 80000 | 80000 | 80000 | 25300 |
| n (Samp) | 171 | 23 | 171 | 23 | 171 | 14 |
| n (Patient) | 171 | 23 | 171 | 23 | 171 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.84 | 0.73 | 0.86 | 0.83 | 0.71 | 0.86 | 0.77 | 0.70 | 0.76 |
| SE | 0.047 | 0.081 | 0.050 | 0.048 | 0.082 | 0.051 | 0.070 | 0.11 | 0.076 |
| p | 5.5E−13 | 0.0051 | 4.7E−13 | 7.0E−12 | 0.012 | 1.8E−12 | 1.1E−4 | 0.077 | 5.1E−4 |
| nCohort 1 | 222 | 373 | 171 | 222 | 373 | 171 | 222 | 373 | 171 |
| nCohort 2 | 30 | 13 | 23 | 30 | 13 | 23 | 16 | 7 | 14 |
| Cutoff 1 | 9450 | 7480 | 11000 | 9450 | 6410 | 11000 | 6410 | 8560 | 8690 |
| Sens 1 | 70% | 77% | 74% | 70% | 77% | 74% | 75% | 71% | 71% |
| Spec 1 | 80% | 66% | 85% | 80% | 61% | 85% | 72% | 70% | 80% |
| Cutoff 2 | 8150 | 6410 | 8710 | 8150 | 5190 | 8710 | 6150 | 6410 | 3840 |
| Sens 2 | 80% | 85% | 83% | 80% | 85% | 83% | 81% | 86% | 86% |
| Spec 2 | 77% | 61% | 80% | 77% | 53% | 80% | 72% | 61% | 50% |
| Cutoff 3 | 6150 | 2840 | 6150 | 5190 | 2840 | 6150 | 2840 | 2840 | 2840 |
| Sens 3 | 90% | 92% | 91% | 90% | 92% | 91% | 94% | 100% | 93% |
| Spec 3 | 72% | 32% | 71% | 64% | 32% | 71% | 43% | 32% | 38% |
| Cutoff 4 | 5990 | 8650 | 6090 | 5990 | 8650 | 6090 | 5990 | 8650 | 6090 |
| Sens 4 | 90% | 62% | 91% | 87% | 62% | 91% | 81% | 57% | 79% |
| Spec 4 | 70% | 70% | 70% | 70% | 70% | 70% | 70% | 70% | 70% |
| Cutoff 5 | 9450 | 12900 | 8710 | 9450 | 12900 | 8710 | 9450 | 12900 | 8710 |
| Sens 5 | 70% | 46% | 83% | 70% | 46% | 83% | 50% | 43% | 64% |
| Spec 5 | 80% | 80% | 80% | 80% | 80% | 80% | 80% | 80% | 80% |
| Cutoff 6 | 15800 | 17100 | 15800 | 15800 | 17100 | 15800 | 15800 | 17100 | 15800 |
| Sens 6 | 40% | 38% | 43% | 30% | 23% | 35% | 19% | 0% | 21% |
| Spec 6 | 90% | 90% | 90% | 90% | 90% | 90% | 90% | 90% | 90% |
| OR Quart 2 | 1.0 | 0.99 | >1.0 | 1.0 | 0.99 | >1.0 | >2.0 | >1.0 | >3.2 |
| p Value | 1.0 | 0.99 | <1.0 | 1.0 | 0.99 | <1.0 | <0.57 | <0.99 | <0.32 |
| 95% CI of | 0.061 | 0.061 | >0.061 | 0.061 | 0.061 | >0.061 | >0.18 | >0.062 | >0.32 |
| OR Quart2 | 16 | 16 | na | 16 | 16 | na | na | na | na |
| OR Quart 3 | 9.0 | 4.1 | >5.6 | 9.0 | 4.1 | >5.6 | >3.2 | >3.1 | >1.0 |
| p Value | 0.041 | 0.21 | <0.12 | 0.041 | 0.21 | <0.12 | <0.33 | <0.33 | <0.99 |
| 95% CI of | 1.1 | 0.45 | >0.63 | 1.1 | 0.45 | >0.63 | >0.32 | >0.32 | >0.062 |
| OR Quart3 | 74 | 38 | na | 74 | 38 | na | na | na | na |
| OR Quart 4 | 29 | 7.4 | >26 | 29 | 7.4 | >26 | >13 | >3.1 | >12 |
| p Value | 0.0013 | 0.064 | <0.0021 | 0.0013 | 0.064 | <0.0021 | <0.015 | <0.33 | <0.019 |
| 95% CI of | 3.7 | 0.89 | >3.2 | 3.7 | 0.89 | >3.2 | >1.7 | >0.32 | >1.5 |
| OR Quart4 | 220 | 61 | na | 220 | 61 | na | na | na | na |

Immunoglogulin G2

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 9710 | 38600 | 9710 | 36400 | 9710 | 27800 |
| Average | 20300 | 71300 | 20300 | 65400 | 20300 | 46400 |
| Stdev | 34400 | 73800 | 34400 | 72200 | 34400 | 58500 |
| p (t-test) | | 7.4E−10 | | 3.2E−8 | | 0.0059 |
| Min | 119 | 2380 | 119 | 2380 | 119 | 4940 |
| Max | 240000 | 240000 | 240000 | 240000 | 240000 | 240000 |
| n (Samp) | 222 | 30 | 222 | 30 | 222 | 16 |
| n (Patient) | 222 | 30 | 222 | 30 | 222 | 16 |
| sCr only | | | | | | |
| Median | 13000 | 52100 | 13000 | 36800 | 13000 | 20100 |
| Average | 26100 | 73800 | 26100 | 62100 | 26100 | 62000 |
| Stdev | 40800 | 71300 | 40800 | 67800 | 40800 | 84800 |
| p (t-test) | | 7.0E−5 | | 0.0025 | | 0.025 |
| Min | 119 | 2380 | 119 | 2380 | 119 | 4940 |
| Max | 240000 | 240000 | 240000 | 240000 | 240000 | 240000 |
| n (Samp) | 373 | 13 | 373 | 13 | 373 | 7 |
| n (Patient) | 373 | 13 | 373 | 13 | 373 | 7 |
| UO only | | | | | | |
| Median | 10700 | 40500 | 10700 | 40500 | 10700 | 35700 |
| Average | 22500 | 81500 | 22500 | 77900 | 22500 | 54600 |
| Stdev | 39100 | 84000 | 39100 | 81100 | 39100 | 62500 |
| p (t-test) | | 4.1E−8 | | 1.7E−7 | | 0.0056 |
| Min | 334 | 8300 | 334 | 8300 | 334 | 8300 |
| Max | 240000 | 240000 | 240000 | 240000 | 240000 | 240000 |
| n (Samp) | 171 | 23 | 171 | 23 | 171 | 14 |
| n (Patient) | 171 | 23 | 171 | 23 | 171 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.80 | 0.73 | 0.81 | 0.79 | 0.70 | 0.81 | 0.74 | 0.65 | 0.76 |
| SE | 0.050 | 0.081 | 0.056 | 0.051 | 0.083 | 0.056 | 0.073 | 0.11 | 0.077 |
| p | 3.6E−9 | 0.0041 | 1.7E−8 | 1.2E−8 | 0.018 | 2.2E−8 | 9.4E−4 | 0.18 | 8.5E−4 |
| nCohort 1 | 222 | 373 | 171 | 222 | 373 | 171 | 222 | 373 | 171 |
| nCohort 2 | 30 | 13 | 23 | 30 | 13 | 23 | 16 | 7 | 14 |
| Cutoff 1 | 19300 | 20100 | 14800 | 19300 | 13300 | 14800 | 13700 | 13300 | 14800 |
| Sens 1 | 70% | 77% | 74% | 70% | 77% | 74% | 75% | 71% | 71% |
| Spec 1 | 73% | 67% | 65% | 73% | 52% | 65% | 64% | 52% | 65% |
| Cutoff 2 | 14500 | 8240 | 14500 | 14500 | 8240 | 14500 | 9820 | 8240 | 9820 |
| Sens 2 | 80% | 85% | 83% | 80% | 85% | 83% | 81% | 86% | 86% |
| Spec 2 | 66% | 33% | 64% | 66% | 33% | 64% | 50% | 33% | 46% |
| Cutoff 3 | 8830 | 4910 | 12300 | 8830 | 4910 | 12300 | 8240 | 4910 | 8540 |
| Sens 3 | 90% | 92% | 91% | 90% | 92% | 91% | 94% | 100% | 93% |
| Spec 3 | 48% | 19% | 54% | 48% | 19% | 54% | 45% | 19% | 42% |
| Cutoff 4 | 17500 | 21800 | 18100 | 17500 | 21800 | 18100 | 17500 | 21800 | 18100 |
| Sens 4 | 70% | 69% | 70% | 70% | 62% | 70% | 56% | 43% | 57% |
| Spec 4 | 70% | 70% | 70% | 70% | 70% | 70% | 70% | 70% | 70% |
| Cutoff 5 | 23700 | 31900 | 22900 | 23700 | 31900 | 22900 | 23700 | 31900 | 22900 |
| Sens 5 | 63% | 62% | 65% | 60% | 54% | 65% | 50% | 43% | 57% |
| Spec 5 | 80% | 80% | 80% | 80% | 80% | 80% | 80% | 80% | 80% |
| Cutoff 6 | 40900 | 52000 | 44400 | 40900 | 52000 | 44400 | 40900 | 52000 | 44400 |
| Sens 6 | 47% | 54% | 48% | 43% | 46% | 48% | 31% | 43% | 36% |
| Spec 6 | 90% | 90% | 90% | 90% | 90% | 90% | 90% | 90% | 90% |
| OR Quart 2 | 3.1 | 0.49 | >3.1 | 3.1 | 0.49 | >3.1 | >4.2 | 1.0 | >3.2 |
| p Value | 0.33 | 0.56 | <0.33 | 0.33 | 0.56 | <0.33 | <0.20 | 1.0 | <0.32 |
| 95% CI of | 0.31 | 0.044 | >0.31 | 0.31 | 0.044 | >0.31 | >0.46 | 0.062 | >0.32 |
| OR Quart2 | 31 | 5.5 | na | 31 | 5.5 | na | na | 16 | na |
| OR Quart 3 | 7.8 | 1.0 | >5.6 | 9.0 | 1.5 | >5.6 | >4.3 | 2.0 | >3.2 |
| p Value | 0.059 | 1.0 | <0.12 | 0.041 | 0.65 | <0.12 | <0.20 | 0.57 | <0.32 |
| 95% CI of | 0.92 | 0.14 | >0.63 | 1.1 | 0.25 | >0.63 | >0.47 | 0.18 | >0.32 |
| OR Quart3 | 65 | 7.2 | na | 74 | 9.3 | na | na | 23 | na |
| OR Quart 4 | 27 | 4.2 | >21 | 25 | 3.7 | >21 | >9.1 | 3.1 | >9.4 |
| p Value | 0.0017 | 0.073 | <0.0039 | 0.0021 | 0.11 | <0.0039 | <0.041 | 0.34 | <0.038 |
| 95% CI of | 3.5 | 0.87 | >2.7 | 3.2 | 0.74 | >2.7 | >1.1 | 0.31 | >1.1 |
| OR Quart4 | 210 | 20 | na | 190 | 18 | na | na | 30 | na |

Interleukin-11

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 129 | 344 | 129 | 270 | 129 | 266 |
| Average | 208 | 537 | 208 | 472 | 208 | 492 |
| Stdev | 256 | 608 | 256 | 560 | 256 | 637 |
| p (t-test) | | 2.2E−7 | | 1.5E−5 | | 2.6E−4 |
| Min | 0.154 | 48.0 | 0.154 | 48.0 | 0.154 | 85.8 |
| Max | 1980 | 2900 | 1980 | 2590 | 1980 | 2140 |
| n (Samp) | 223 | 30 | 223 | 30 | 223 | 16 |
| n (Patient) | 223 | 30 | 223 | 30 | 223 | 16 |
| sCr only | | | | | | |
| Median | 174 | 295 | 174 | 219 | 174 | 212 |
| Average | 285 | 322 | 285 | 313 | 285 | 237 |
| Stdev | 315 | 259 | 315 | 262 | 315 | 112 |
| p (t-test) | | 0.67 | | 0.75 | | 0.69 |
| Min | 0.154 | 48.0 | 0.154 | 48.0 | 0.154 | 115 |
| Max | 2260 | 1040 | 2260 | 1040 | 2260 | 436 |
| n (Samp) | 375 | 13 | 375 | 13 | 375 | 7 |
| n (Patient) | 375 | 13 | 375 | 13 | 375 | 7 |
| UO only | | | | | | |
| Median | 179 | 396 | 179 | 295 | 179 | 321 |
| Average | 263 | 664 | 263 | 585 | 263 | 582 |
| Stdev | 283 | 683 | 283 | 640 | 283 | 682 |
| p (t-test) | | 7.1E−7 | | 3.7E−5 | | 5.7E−4 |
| Min | 2.83 | 90.9 | 2.83 | 90.9 | 2.83 | 85.8 |
| Max | 1980 | 2900 | 1980 | 2590 | 1980 | 2140 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.77 | 0.60 | 0.76 | 0.74 | 0.59 | 0.71 | 0.73 | 0.57 | 0.68 |
| SE | 0.052 | 0.084 | 0.061 | 0.054 | 0.084 | 0.063 | 0.074 | 0.11 | 0.081 |
| p | 1.7E−7 | 0.23 | 2.4E−5 | 1.0E−5 | 0.29 | 0.0010 | 0.0019 | 0.54 | 0.026 |
| nCohort 1 | 223 | 375 | 173 | 223 | 375 | 173 | 223 | 375 | 173 |
| nCohort 2 | 30 | 13 | 23 | 30 | 13 | 23 | 16 | 7 | 14 |
| Cutoff 1 | 230 | 135 | 250 | 180 | 135 | 212 | 143 | 179 | 235 |
| Sens 1 | 70% | 77% | 74% | 70% | 77% | 74% | 75% | 71% | 71% |
| Spec 1 | 73% | 40% | 64% | 64% | 40% | 58% | 57% | 52% | 62% |
| Cutoff 2 | 143 | 114 | 212 | 135 | 114 | 143 | 135 | 135 | 114 |
| Sens 2 | 80% | 85% | 83% | 80% | 85% | 83% | 81% | 86% | 86% |
| Spec 2 | 57% | 33% | 58% | 55% | 33% | 44% | 55% | 40% | 34% |
| Cutoff 3 | 114 | 110 | 128 | 110 | 110 | 114 | 85.8 | 114 | 89.1 |
| Sens 3 | 90% | 92% | 91% | 90% | 92% | 91% | 94% | 100% | 93% |
| Spec 3 | 47% | 32% | 36% | 46% | 32% | 34% | 37% | 33% | 24% |
| Cutoff 4 | 212 | 301 | 299 | 212 | 301 | 299 | 212 | 301 | 299 |
| Sens 4 | 77% | 38% | 61% | 67% | 38% | 48% | 62% | 14% | 50% |
| Spec 4 | 70% | 70% | 71% | 70% | 70% | 71% | 70% | 70% | 71% |
| Cutoff 5 | 301 | 461 | 383 | 301 | 461 | 383 | 301 | 461 | 383 |
| Sens 5 | 53% | 15% | 52% | 43% | 15% | 43% | 44% | 0% | 43% |
| Spec 5 | 80% | 80% | 80% | 80% | 80% | 80% | 80% | 80% | 80% |
| Cutoff 6 | 487 | 694 | 569 | 487 | 694 | 569 | 487 | 694 | 569 |
| Sens 6 | 33% | 8% | 39% | 30% | 8% | 35% | 19% | 0% | 29% |
| Spec 6 | 90% | 90% | 90% | 90% | 90% | 90% | 90% | 90% | 90% |
| OR Quart 2 | 4.2 | 3.1 | 3.1 | 4.2 | 3.1 | 2.1 | >3.1 | >2.0 | 0.98 |
| p Value | 0.20 | 0.34 | 0.33 | 0.20 | 0.34 | 0.41 | <0.33 | <0.57 | 0.98 |
| 95% CI of | 0.46 | 0.31 | 0.31 | 0.46 | 0.31 | 0.36 | >0.31 | >0.18 | 0.13 |
| OR Quart2 | 39 | 30 | 31 | 39 | 30 | 12 | na | na | 7.3 |
| OR Quart 3 | 7.8 | 5.2 | 8.0 | 12 | 5.2 | 3.3 | >5.4 | >4.2 | 1.5 |
| p Value | 0.059 | 0.13 | 0.056 | 0.021 | 0.13 | 0.16 | <0.13 | <0.20 | 0.67 |
| 95% CI of | 0.92 | 0.60 | 0.95 | 1.4 | 0.60 | 0.63 | >0.61 | >0.46 | 0.24 |
| OR Quart3 | 65 | 46 | 68 | 94 | 46 | 17 | na | na | 9.4 |
| OR Quart 4 | 24 | 4.1 | 16 | 19 | 4.1 | 6.8 | >9.1 | >1.0 | 3.8 |
| p Value | 0.0023 | 0.21 | 0.0098 | 0.0051 | 0.21 | 0.016 | <0.041 | <1.0 | 0.10 |
| 95% CI of | 3.1 | 0.45 | 1.9 | 2.4 | 0.45 | 1.4 | >1.1 | >0.062 | 0.76 |
| OR Quart4 | 190 | 38 | 130 | 150 | 38 | 33 | na | na | 20 |

Interleukin-2 receptor alpha chain

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 634 | 1730 | 634 | 1720 | 634 | 980 |
| Average | 1020 | 2350 | 1020 | 2180 | 1020 | 2070 |
| Stdev | 1210 | 2340 | 1210 | 2280 | 1210 | 2470 |
| p (t-test) | | 1.6E−6 | | 2.4E−5 | | 0.0026 |
| Min | 0.0708 | 0.118 | 0.0708 | 0.118 | 0.0708 | 437 |
| Max | 10400 | 10400 | 10400 | 10400 | 10400 | 10400 |
| n (Samp) | 223 | 30 | 223 | 30 | 223 | 16 |
| n (Patient) | 223 | 30 | 223 | 30 | 223 | 16 |
| sCr only | | | | | | |
| Median | 880 | 1300 | 880 | 1300 | 880 | 2450 |
| Average | 1390 | 2050 | 1390 | 2040 | 1390 | 2570 |
| Stdev | 1620 | 2100 | 1620 | 2100 | 1620 | 2370 |
| p (t-test) | | 0.16 | | 0.16 | | 0.059 |
| Min | 0.0708 | 0.118 | 0.0708 | 0.118 | 0.0708 | 437 |
| Max | 10400 | 7190 | 10400 | 7190 | 10400 | 7190 |
| n (Samp) | 375 | 13 | 375 | 13 | 375 | 7 |
| n (Patient) | 375 | 13 | 375 | 13 | 375 | 7 |
| UO only | | | | | | |
| Median | 767 | 2490 | 767 | 2200 | 767 | 1140 |
| Average | 1130 | 2710 | 1130 | 2480 | 1130 | 2170 |
| Stdev | 1300 | 2450 | 1300 | 2410 | 1300 | 2610 |
| p (t-test) | | 3.0E−6 | | 5.1E−5 | | 0.0095 |
| Min | 0.116 | 50.9 | 0.116 | 50.9 | 0.116 | 437 |
| Max | 10400 | 10400 | 10400 | 10400 | 10400 | 10400 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.70 | 0.59 | 0.74 | 0.69 | 0.59 | 0.73 | 0.70 | 0.69 | 0.68 |
| SE | 0.056 | 0.084 | 0.061 | 0.056 | 0.084 | 0.063 | 0.075 | 0.11 | 0.082 |
| p | 2.8E−4 | 0.31 | 7.2E−5 | 6.9E−4 | 0.31 | 3.1E−4 | 0.0094 | 0.098 | 0.030 |
| nCohort 1 | 223 | 375 | 173 | 223 | 375 | 173 | 223 | 375 | 173 |
| nCohort 2 | 30 | 13 | 23 | 30 | 13 | 23 | 16 | 7 | 14 |
| Cutoff 1 | 750 | 493 | 1070 | 750 | 493 | 954 | 712 | 960 | 750 |
| Sens 1 | 70% | 77% | 74% | 70% | 77% | 74% | 75% | 71% | 71% |
| Spec 1 | 53% | 32% | 63% | 53% | 32% | 59% | 52% | 53% | 49% |
| Cutoff 2 | 491 | 420 | 491 | 491 | 420 | 491 | 594 | 594 | 491 |
| Sens 2 | 80% | 85% | 83% | 80% | 85% | 83% | 81% | 86% | 86% |
| Spec 2 | 44% | 29% | 41% | 44% | 29% | 41% | 48% | 38% | 41% |
| Cutoff 3 | 384 | 55.0 | 411 | 384 | 55.0 | 411 | 484 | 420 | 484 |
| Sens 3 | 90% | 92% | 91% | 90% | 92% | 91% | 94% | 100% | 93% |
| Spec 3 | 40% | 9% | 38% | 40% | 9% | 38% | 44% | 29% | 41% |
| Cutoff 4 | 1250 | 1600 | 1270 | 1250 | 1600 | 1270 | 1250 | 1600 | 1270 |
| Sens 4 | 60% | 46% | 65% | 57% | 46% | 61% | 44% | 57% | 50% |
| Spec 4 | 70% | 70% | 71% | 70% | 70% | 71% | 70% | 70% | 71% |
| Cutoff 5 | 1690 | 2070 | 1890 | 1690 | 2070 | 1890 | 1690 | 2070 | 1890 |
| Sens 5 | 50% | 46% | 57% | 50% | 46% | 57% | 44% | 57% | 43% |
| Spec 5 | 80% | 80% | 80% | 80% | 80% | 80% | 80% | 80% | 80% |
| Cutoff 6 | 2530 | 3270 | 2620 | 2530 | 3270 | 2620 | 2530 | 3270 | 2620 |
| Sens 6 | 37% | 23% | 43% | 27% | 23% | 30% | 19% | 29% | 21% |
| Spec 6 | 90% | 90% | 90% | 90% | 90% | 90% | 90% | 90% | 90% |
| OR Quart 2 | 2.1 | 1.5 | 5.5 | 2.1 | 1.5 | 5.5 | >4.2 | >2.0 | >5.5 |
| p Value | 0.31 | 0.65 | 0.13 | 0.31 | 0.65 | 0.13 | <0.20 | <0.57 | <0.13 |
| 95% CI of | 0.50 | 0.25 | 0.61 | 0.50 | 0.25 | 0.61 | >0.46 | >0.18 | >0.61 |
| OR Quart2 | 8.8 | 9.3 | 49 | 8.8 | 9.3 | 49 | na | na | na |
| OR Quart 3 | 2.1 | 1.0 | 4.3 | 2.1 | 1.0 | 4.3 | >5.4 | >1.0 | >3.1 |
| p Value | 0.31 | 1.0 | 0.20 | 0.31 | 1.0 | 0.20 | <0.13 | <0.99 | <0.33 |
| 95% CI of | 0.50 | 0.14 | 0.46 | 0.50 | 0.14 | 0.46 | >0.61 | >0.062 | >0.31 |
| OR Quart3 | 8.8 | 7.2 | 40 | 8.8 | 7.2 | 40 | na | na | na |
| OR Quart 4 | 6.1 | 3.1 | 17 | 6.1 | 3.1 | 17 | >7.8 | >4.1 | >6.7 |
| p Value | 0.0061 | 0.17 | 0.0072 | 0.0061 | 0.17 | 0.0072 | <0.059 | <0.21 | <0.083 |
| 95% CI of | 1.7 | 0.62 | 2.2 | 1.7 | 0.62 | 2.2 | >0.93 | >0.45 | >0.78 |
| OR Quart4 | 22 | 16 | 140 | 22 | 16 | 140 | na | na | na |

Neutrophil collagenase

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3750 | 21100 | 3750 | 20800 | 3750 | 20800 |
| Average | 17900 | 92500 | 17900 | 89700 | 17900 | 60700 |
| Stdev | 56800 | 164000 | 56800 | 165000 | 56800 | 137000 |
| p (t-test) | | 4.4E−7 | | 1.1E−6 | | 0.0059 |
| Min | 0.114 | 25.8 | 0.114 | 25.8 | 0.114 | 580 |
| Max | 670000 | 649000 | 670000 | 649000 | 670000 | 625000 |
| n (Samp) | 235 | 34 | 235 | 34 | 235 | 20 |
| n (Patient) | 235 | 34 | 235 | 34 | 235 | 20 |
| sCr only | | | | | | |
| Median | 6390 | 10800 | 6390 | 10800 | 6390 | 21100 |
| Average | 25600 | 113000 | 25600 | 86800 | 25600 | 37900 |
| Stdev | 65500 | 215000 | 65500 | 175000 | 65500 | 47300 |
| p (t-test) | | 5.6E−6 | | 7.7E−4 | | 0.54 |
| Min | 0.114 | 25.8 | 0.114 | 25.8 | 0.114 | 2660 |
| Max | 670000 | 649000 | 670000 | 649000 | 670000 | 151000 |
| n (Samp) | 398 | 17 | 398 | 17 | 398 | 11 |
| n (Patient) | 398 | 17 | 398 | 17 | 398 | 11 |
| UO only | | | | | | |
| Median | 4250 | 48900 | 4250 | 41500 | 4250 | 21000 |
| Average | 14300 | 131000 | 14300 | 110000 | 14300 | 114000 |
| Stdev | 32500 | 200000 | 32500 | 178000 | 32500 | 215000 |
| p (t-test) | | 2.8E−12 | | 1.9E−10 | | 3.3E−8 |
| Min | 0.114 | 580 | 0.114 | 580 | 0.114 | 580 |
| Max | 300000 | 649000 | 300000 | 649000 | 300000 | 649000 |
| n (Samp) | 190 | 24 | 190 | 24 | 190 | 15 |
| n (Patient) | 190 | 24 | 190 | 24 | 190 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.75 | 0.61 | 0.82 | 0.74 | 0.60 | 0.81 | 0.75 | 0.67 | 0.76 |
| SE | 0.051 | 0.074 | 0.054 | 0.051 | 0.074 | 0.055 | 0.065 | 0.091 | 0.074 |
| p | 1.2E−6 | 0.13 | 1.8E−9 | 3.1E−6 | 0.18 | 1.4E−8 | 1.0E−4 | 0.057 | 3.2E−4 |
| nCohort 1 | 235 | 398 | 190 | 235 | 398 | 190 | 235 | 398 | 190 |
| nCohort 2 | 34 | 17 | 24 | 34 | 17 | 24 | 20 | 11 | 15 |
| Cutoff 1 | 10800 | 5530 | 20000 | 7010 | 5530 | 14600 | 7010 | 6050 | 7160 |
| Sens 1 | 71% | 71% | 71% | 71% | 71% | 71% | 70% | 73% | 73% |
| Spec 1 | 72% | 46% | 82% | 64% | 46% | 76% | 64% | 48% | 63% |
| Cutoff 2 | 3260 | 3170 | 11900 | 3260 | 3000 | 7160 | 5930 | 5930 | 5930 |
| Sens 2 | 82% | 82% | 83% | 82% | 82% | 83% | 80% | 82% | 80% |
| Spec 2 | 46% | 33% | 74% | 46% | 32% | 63% | 60% | 48% | 58% |
| Cutoff 3 | 2710 | 112 | 3260 | 2630 | 112 | 3260 | 3260 | 3170 | 3260 |
| Sens 3 | 91% | 94% | 92% | 91% | 94% | 92% | 90% | 91% | 93% |
| Spec 3 | 42% | 1% | 42% | 41% | 1% | 42% | 46% | 33% | 42% |
| Cutoff 4 | 9820 | 16300 | 9300 | 9820 | 16300 | 9300 | 9820 | 16300 | 9300 |
| Sens 4 | 71% | 47% | 83% | 68% | 47% | 79% | 65% | 55% | 67% |
| Spec 4 | 70% | 70% | 70% | 70% | 70% | 70% | 70% | 70% | 70% |
| Cutoff 5 | 18200 | 27300 | 19400 | 18200 | 27300 | 19400 | 18200 | 27300 | 19400 |
| Sens 5 | 59% | 35% | 71% | 56% | 35% | 67% | 55% | 36% | 60% |
| Spec 5 | 80% | 80% | 80% | 80% | 80% | 80% | 80% | 80% | 80% |
| Cutoff 6 | 33500 | 55700 | 32200 | 33500 | 55700 | 32200 | 33500 | 55700 | 32200 |
| Sens 6 | 41% | 29% | 54% | 41% | 29% | 54% | 35% | 27% | 47% |
| Spec 6 | 90% | 90% | 90% | 90% | 90% | 90% | 90% | 90% | 90% |
| OR Quart 2 | 1.4 | 3.1 | 2.0 | 1.4 | 3.1 | 2.0 | 2.0 | >4.2 | 1.0 |
| p Value | 0.70 | 0.17 | 0.58 | 0.70 | 0.17 | 0.58 | 0.58 | <0.21 | 1.0 |
| 95% CI of | 0.29 | 0.61 | 0.18 | 0.29 | 0.61 | 0.18 | 0.18 | >0.46 | 0.061 |
| OR Quart2 | 6.3 | 16 | 23 | 6.3 | 16 | 23 | 23 | na | 16 |
| OR Quart 3 | 2.5 | 0.99 | 4.2 | 2.9 | 0.99 | 5.4 | 6.4 | >2.0 | 4.3 |
| p Value | 0.20 | 0.99 | 0.20 | 0.13 | 0.99 | 0.13 | 0.090 | <0.56 | 0.20 |
| 95% CI of | 0.62 | 0.14 | 0.46 | 0.73 | 0.14 | 0.61 | 0.75 | >0.18 | 0.46 |
| OR Quart3 | 10 | 7.2 | 39 | 11 | 7.2 | 48 | 55 | na | 39 |
| OR Quart 4 | 8.9 | 3.6 | 24 | 8.3 | 3.6 | 22 | 13 | >5.2 | 10 |
| p Value | 7.5E−4 | 0.11 | 0.0025 | 0.0011 | 0.11 | 0.0034 | 0.016 | <0.14 | 0.029 |
| 95% CI of | 2.5 | 0.74 | 3.0 | 2.3 | 0.74 | 2.8 | 1.6 | >0.60 | 1.3 |
| OR Quart4 | 32 | 18 | 190 | 30 | 18 | 170 | 100 | na | 86 |

Protransforming growth factor alpha

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 6.37 | 16.8 | 6.37 | 15.7 | 6.37 | 14.5 |
| Average | 13.8 | 25.7 | 13.8 | 24.3 | 13.8 | 25.1 |
| Stdev | 31.8 | 23.8 | 31.8 | 23.3 | 31.8 | 28.7 |
| p (t-test) | | 0.049 | | 0.081 | | 0.17 |
| Min | 0.00574 | 2.98 | 0.00574 | 1.41 | 0.00574 | 5.53 |
| Max | 361 | 122 | 361 | 122 | 361 | 122 |
| n (Samp) | 223 | 30 | 223 | 30 | 223 | 16 |
| n (Patient) | 223 | 30 | 223 | 30 | 223 | 16 |
| sCr only | | | | | | |
| Median | 9.29 | 15.5 | 9.29 | 15.5 | 9.29 | 15.9 |
| Average | 16.5 | 19.3 | 16.5 | 18.4 | 16.5 | 22.4 |
| Stdev | 29.1 | 13.5 | 29.1 | 13.8 | 29.1 | 17.1 |
| p (t-test) | | 0.73 | | 0.81 | | 0.59 |
| Min | 0.00574 | 2.98 | 0.00574 | 1.41 | 0.00574 | 5.53 |
| Max | 361 | 50.4 | 361 | 50.4 | 361 | 50.4 |
| n (Samp) | 375 | 13 | 375 | 13 | 375 | 7 |
| n (Patient) | 375 | 13 | 375 | 13 | 375 | 7 |
| UO only | | | | | | |
| Median | 7.74 | 18.0 | 7.74 | 17.8 | 7.74 | 14.5 |
| Average | 16.2 | 30.0 | 16.2 | 26.9 | 16.2 | 25.0 |
| Stdev | 35.5 | 26.9 | 35.5 | 25.4 | 35.5 | 30.2 |
| p (t-test) | | 0.073 | | 0.16 | | 0.37 |
| Min | 0.00662 | 5.53 | 0.00662 | 5.53 | 0.00662 | 5.53 |
| Max | 361 | 122 | 361 | 122 | 361 | 122 |
| n (Samp) | 173 | 23 | 173 | 23 | 173 | 14 |
| n (Patient) | 173 | 23 | 173 | 23 | 173 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.80 | 0.67 | 0.79 | 0.78 | 0.65 | 0.78 | 0.77 | 0.69 | 0.73 |
| SE | 0.050 | 0.083 | 0.058 | 0.051 | 0.084 | 0.059 | 0.070 | 0.11 | 0.079 |
| p | 2.3E−9 | 0.038 | 6.7E−7 | 3.5E−8 | 0.079 | 3.0E−6 | 8.9E−5 | 0.086 | 0.0037 |
| nCohort 1 | 223 | 375 | 173 | 223 | 375 | 173 | 223 | 375 | 173 |
| nCohort 2 | 30 | 13 | 23 | 30 | 13 | 23 | 16 | 7 | 14 |
| Cutoff 1 | 13.1 | 12.2 | 13.6 | 12.7 | 11.3 | 12.7 | 11.4 | 12.6 | 12.4 |
| Sens 1 | 70% | 77% | 74% | 70% | 77% | 74% | 75% | 71% | 71% |
| Spec 1 | 78% | 64% | 73% | 77% | 59% | 72% | 72% | 65% | 72% |
| Cutoff 2 | 12.1 | 11.3 | 12.1 | 11.1 | 9.43 | 11.4 | 9.43 | 9.43 | 8.10 |
| Sens 2 | 80% | 85% | 83% | 80% | 85% | 83% | 81% | 86% | 86% |
| Spec 2 | 76% | 59% | 71% | 72% | 51% | 69% | 65% | 51% | 52% |
| Cutoff 3 | 8.13 | 5.52 | 8.10 | 8.13 | 5.52 | 8.10 | 6.18 | 5.52 | 6.18 |
| Sens 3 | 90% | 92% | 91% | 90% | 92% | 91% | 94% | 100% | 93% |
| Spec 3 | 59% | 31% | 52% | 59% | 31% | 52% | 48% | 31% | 40% |
| Cutoff 4 | 10.8 | 15.5 | 11.8 | 10.8 | 15.5 | 11.8 | 10.8 | 15.5 | 11.8 |
| Sens 4 | 83% | 54% | 83% | 80% | 54% | 78% | 75% | 57% | 71% |
| Spec 4 | 70% | 70% | 71% | 70% | 70% | 71% | 70% | 70% | 71% |
| Cutoff 5 | 14.8 | 20.2 | 17.9 | 14.8 | 20.2 | 17.9 | 14.8 | 20.2 | 17.9 |
| Sens 5 | 57% | 31% | 52% | 53% | 31% | 48% | 44% | 43% | 36% |
| Spec 5 | 80% | 80% | 80% | 80% | 80% | 80% | 80% | 80% | 80% |
| Cutoff 6 | 25.0 | 33.1 | 28.7 | 25.0 | 33.1 | 28.7 | 25.0 | 33.1 | 28.7 |
| Sens 6 | 30% | 15% | 35% | 27% | 15% | 30% | 25% | 29% | 21% |
| Spec 6 | 90% | 90% | 90% | 90% | 90% | 90% | 90% | 90% | 90% |
| OR Quart 2 | 2.0 | 1.0 | >3.2 | 2.0 | 1.0 | >3.2 | >2.0 | >1.0 | >3.1 |
| p Value | 0.57 | 1.0 | <0.32 | 0.57 | 1.0 | <0.32 | <0.57 | <1.0 | <0.33 |
| 95% CI of | 0.18 | 0.062 | >0.32 | 0.18 | 0.062 | >0.32 | >0.18 | >0.062 | >0.31 |
| OR Quart2 | 23 | 16 | na | 23 | 16 | na | na | na | na |
| OR Quart 3 | 10 | 6.3 | >8.2 | 10 | 7.5 | >9.6 | >4.2 | >3.1 | >6.7 |
| p Value | 0.029 | 0.090 | <0.054 | 0.029 | 0.062 | <0.037 | <0.20 | <0.33 | <0.083 |
| 95% CI of | 1.3 | 0.75 | >0.97 | 1.3 | 0.90 | >1.1 | >0.46 | >0.32 | >0.78 |
| OR Quart3 | 84 | 54 | na | 84 | 62 | na | na | na | na |
| OR Quart 4 | 24 | 5.2 | >18 | 24 | 4.1 | >16 | >12 | >3.1 | >5.5 |
| p Value | 0.0023 | 0.13 | <0.0068 | 0.0023 | 0.21 | <0.0093 | <0.021 | <0.34 | <0.13 |
| 95% CI of | 3.1 | 0.60 | >2.2 | 3.1 | 0.45 | >2.0 | >1.5 | >0.31 | >0.61 |
| OR Quart4 | 190 | 46 | na | 190 | 38 | na | na | na | na |

CA 15-3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 434 | 434 | 434 | 434 | 434 | 434 |
| Average | 327 | 448 | 327 | 426 | 327 | 393 |
| Stdev | 253 | 156 | 253 | 184 | 253 | 109 |
| p (t-test) | | 0.050 | | 0.11 | | 0.36 |
| Min | 3.03 | 63.1 | 3.03 | 39.8 | 3.03 | 63.1 |
| Max | 784 | 784 | 784 | 784 | 784 | 434 |
| n (Samp) | 123 | 18 | 123 | 18 | 123 | 13 |
| n (Patient) | 123 | 18 | 123 | 18 | 123 | 13 |
| sCr only | | | | | | |
| Median | 434 | 434 | 434 | 434 | 434 | 434 |
| Average | 379 | 451 | 379 | 415 | 379 | 413 |
| Stdev | 241 | 121 | 241 | 174 | 241 | 59.2 |
| p (t-test) | | 0.33 | | 0.63 | | 0.69 |
| Min | 3.03 | 266 | 3.03 | 39.8 | 3.03 | 266 |
| Max | 784 | 784 | 784 | 784 | 784 | 434 |
| n (Samp) | 210 | 11 | 210 | 11 | 210 | 8 |
| n (Patient) | 210 | 11 | 210 | 11 | 210 | 8 |
| UO only | | | | | | |
| Median | 434 | 434 | 434 | 434 | 434 | 434 |
| Average | 338 | 441 | 338 | 441 | 338 | 388 |
| Stdev | 243 | 172 | 243 | 172 | 243 | 131 |
| p (t-test) | | 0.19 | | 0.19 | | 0.57 |
| Min | 3.03 | 63.1 | 3.03 | 63.1 | 3.03 | 63.1 |
| Max | 784 | 784 | 784 | 784 | 784 | 434 |
| n (Samp) | 119 | 10 | 119 | 10 | 119 | 8 |
| n (Patient) | 119 | 10 | 119 | 10 | 119 | 8 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.65 | 0.58 | 0.64 | 0.63 | 0.54 | 0.64 | 0.60 | 0.54 | 0.58 |
| SE | 0.074 | 0.092 | 0.098 | 0.075 | 0.091 | 0.098 | 0.087 | 0.11 | 0.11 |
| p | 0.037 | 0.38 | 0.14 | 0.083 | 0.65 | 0.14 | 0.27 | 0.70 | 0.49 |
| nCohort 1 | 123 | 210 | 119 | 123 | 210 | 119 | 123 | 210 | 119 |
| nCohort 2 | 18 | 11 | 10 | 18 | 11 | 10 | 13 | 8 | 8 |
| Cutoff 1 | 413 | 413 | 363 | 413 | 413 | 363 | 413 | 413 | 363 |
| Sens 1 | 89% | 91% | 90% | 83% | 82% | 90% | 85% | 88% | 88% |
| Spec 1 | 42% | 32% | 37% | 42% | 32% | 37% | 42% | 32% | 37% |
| Cutoff 2 | 413 | 413 | 363 | 413 | 413 | 363 | 413 | 413 | 363 |
| Sens 2 | 89% | 91% | 90% | 83% | 82% | 90% | 85% | 88% | 88% |
| Spec 2 | 42% | 32% | 37% | 42% | 32% | 37% | 42% | 32% | 37% |
| Cutoff 3 | 248 | 413 | 363 | 61.3 | 253 | 363 | 248 | 253 | 61.3 |
| Sens 3 | 94% | 91% | 90% | 94% | 91% | 90% | 92% | 100% | 100% |
| Spec 3 | 40% | 32% | 37% | 28% | 29% | 37% | 40% | 29% | 25% |
| Cutoff 4 | 434 | 434 | 434 | 434 | 434 | 434 | 434 | 434 | 434 |
| Sens 4 | 17% | 9% | 20% | 17% | 9% | 20% | 0% | 0% | 0% |
| Spec 4 | 86% | 83% | 87% | 86% | 83% | 87% | 86% | 83% | 87% |
| Cutoff 5 | 434 | 434 | 434 | 434 | 434 | 434 | 434 | 434 | 434 |
| Sens 5 | 17% | 9% | 20% | 17% | 9% | 20% | 0% | 0% | 0% |
| Spec 5 | 86% | 83% | 87% | 86% | 83% | 87% | 86% | 83% | 87% |
| Cutoff 6 | 784 | 784 | 784 | 784 | 784 | 784 | 784 | 784 | 784 |
| Sens 6 | 0% | 0% | 0% | 0% | 0% | 0% | 0% | 0% | 0% |
| Spec 6 | 100% | 100% | 100% | 100% | 100% | 100% | 100% | 100% | 100% |
| OR Quart 2 | >12 | >8.0 | 7.2 | 12 | 6.6 | 7.2 | >10 | >5.4 | 0 |
| p Value | <0.022 | <0.055 | 0.077 | 0.023 | 0.085 | 0.077 | <0.032 | <0.13 | na |
| 95% CI of | >1.4 | >0.95 | 0.81 | 1.4 | 0.77 | 0.81 | >1.2 | >0.61 | na |
| OR Quart2 | na | na | 63 | 99 | 57 | 63 | na | na | na |
| OR Quart 3 | >7.2 | >3.2 | 1.0 | 5.7 | 3.1 | 1.0 | >4.5 | >3.2 | 8.4 |
| p Value | <0.074 | <0.32 | 1.0 | 0.12 | 0.33 | 1.0 | <0.19 | <0.32 | 0.054 |
| 95% CI of | >0.82 | >0.32 | 0.060 | 0.63 | 0.31 | 0.060 | >0.48 | >0.32 | 0.97 |
| OR Quart3 | na | na | 17 | 51 | 31 | 17 | na | na | 73 |
| OR Quart 4 | >3.2 | >1.0 | 2.0 | 3.1 | 0.98 | 2.0 | >1.0 | >0 | 0 |
| p Value | <0.33 | <1.0 | 0.58 | 0.34 | 0.99 | 0.58 | <0.98 | <na | na |
| 95% CI of | >0.31 | >0.061 | 0.17 | 0.31 | 0.060 | 0.17 | >0.062 | >na | na |
| OR Quart4 | na | na | 23 | 31 | 16 | 23 | na | na | na |

TABLE 5
Comparison of marker levels in EDTA samples collected from
Cohort 1 (patients that did not progress beyond RIFLE stage 0) and in EDTA samples
collected from subjects at 0, 24 hours, and 48 hours prior to reaching stage R, I or F in Cohort 2.
C-C motif chemokine 18

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 161 | 203 | 161 | 207 | 161 | 190 |
| Average | 217 | 302 | 217 | 307 | 217 | 310 |
| Stdev | 171 | 316 | 171 | 299 | 171 | 318 |
| p (t-test) | | 0.024 | | 0.013 | | 0.038 |
| Min | 37.8 | 40.9 | 37.8 | 41.6 | 37.8 | 82.0 |
| Max | 1020 | 2110 | 1020 | 1650 | 1020 | 1550 |
| n (Samp) | 121 | 53 | 121 | 53 | 121 | 26 |
| n (Patient) | 87 | 53 | 87 | 53 | 87 | 26 |
| sCr only | | | | | | |
| Median | 180 | 221 | 180 | 279 | 180 | 220 |
| Average | 257 | 264 | 257 | 296 | 257 | 262 |
| Stdev | 251 | 185 | 251 | 166 | 251 | 160 |
| p (t-test) | | 0.92 | | 0.56 | | 0.96 |
| Min | 34.0 | 40.9 | 34.0 | 41.6 | 34.0 | 66.4 |
| Max | 2110 | 848 | 2110 | 679 | 2110 | 548 |
| n (Samp) | 288 | 16 | 288 | 15 | 288 | 10 |
| n (Patient) | 161 | 16 | 161 | 15 | 161 | 10 |
| UO only | | | | | | |
| Median | 167 | 192 | 167 | 201 | 167 | 182 |
| Average | 229 | 303 | 229 | 298 | 229 | 314 |
| Stdev | 174 | 336 | 174 | 297 | 174 | 336 |
| p (t-test) | | 0.063 | | 0.053 | | 0.070 |
| Min | 37.8 | 46.6 | 37.8 | 59.1 | 37.8 | 66.1 |
| Max | 848 | 2110 | 848 | 1650 | 848 | 1550 |
| n (Samp) | 125 | 43 | 125 | 54 | 125 | 23 |
| n (Patient) | 80 | 43 | 80 | 54 | 80 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.61 | 0.57 | 0.58 | 0.60 | 0.63 | 0.57 | 0.60 | 0.57 | 0.56 |
| SE | 0.048 | 0.076 | 0.052 | 0.048 | 0.079 | 0.047 | 0.064 | 0.096 | 0.067 |
| p | 0.024 | 0.33 | 0.11 | 0.039 | 0.10 | 0.14 | 0.10 | 0.48 | 0.33 |
| nCohort 1 | 121 | 288 | 125 | 121 | 288 | 125 | 121 | 288 | 125 |
| nCohort 2 | 53 | 16 | 43 | 53 | 15 | 54 | 26 | 10 | 23 |
| Cutoff 1 | 162 | 167 | 161 | 147 | 169 | 154 | 138 | 186 | 118 |
| Sens 1 | 72% | 75% | 72% | 72% | 73% | 70% | 73% | 70% | 74% |
| Spec 1 | 50% | 45% | 48% | 45% | 46% | 45% | 44% | 53% | 33% |
| Cutoff 2 | 132 | 162 | 132 | 107 | 162 | 107 | 118 | 152 | 105 |
| Sens 2 | 81% | 81% | 81% | 81% | 80% | 81% | 81% | 80% | 83% |
| Spec 2 | 40% | 43% | 38% | 28% | 43% | 25% | 36% | 40% | 24% |
| Cutoff 3 | 99.3 | 114 | 99.3 | 73.3 | 126 | 73.3 | 102 | 102 | 82.0 |
| Sens 3 | 91% | 94% | 91% | 91% | 93% | 91% | 92% | 90% | 91% |
| Spec 3 | 24% | 26% | 20% | 12% | 32% | 10% | 26% | 20% | 14% |
| Cutoff 4 | 228 | 283 | 274 | 228 | 283 | 274 | 228 | 283 | 274 |
| Sens 4 | 47% | 31% | 35% | 43% | 47% | 33% | 46% | 30% | 30% |
| Spec 4 | 70% | 70% | 70% | 70% | 70% | 70% | 70% | 70% | 70% |
| Cutoff 5 | 302 | 361 | 345 | 302 | 361 | 345 | 302 | 361 | 345 |
| Sens 5 | 32% | 19% | 30% | 32% | 33% | 26% | 23% | 20% | 26% |
| Spec 5 | 80% | 80% | 80% | 80% | 80% | 80% | 80% | 80% | 80% |
| Cutoff 6 | 464 | 516 | 484 | 464 | 516 | 484 | 464 | 516 | 484 |
| Sens 6 | 15% | 6% | 14% | 19% | 7% | 17% | 23% | 10% | 22% |
| Spec 6 | 90% | 90% | 90% | 90% | 90% | 90% | 90% | 90% | 90% |
| OR Quart 2 | 1.3 | 1.5 | 1.0 | 1.1 | 4.1 | 0.58 | 2.6 | 0.49 | 0.78 |
| p Value | 0.65 | 0.65 | 1.0 | 0.85 | 0.21 | 0.29 | 0.20 | 0.56 | 0.72 |
| 95% CI of | 0.46 | 0.25 | 0.34 | 0.41 | 0.45 | 0.21 | 0.61 | 0.043 | 0.19 |
| OR Quart 2 | 3.4 | 9.4 | 3.0 | 2.9 | 38 | 1.6 | 11 | 5.5 | 3.2 |
| OR Quart 3 | 2.0 | 3.8 | 2.1 | 1.6 | 4.1 | 1.9 | 3.5 | 2.1 | 1.8 |
| p Value | 0.15 | 0.11 | 0.14 | 0.34 | 0.21 | 0.14 | 0.077 | 0.41 | 0.36 |
| 95% CI of | 0.77 | 0.75 | 0.78 | 0.61 | 0.45 | 0.80 | 0.87 | 0.37 | 0.52 |
| OR Quart 3 | 5.3 | 19 | 5.8 | 4.1 | 38 | 4.7 | 14 | 12 | 6.0 |
| OR Quart 4 | 2.6 | 2.1 | 1.9 | 2.3 | 6.3 | 1.3 | 2.6 | 1.5 | 1.2 |
| p Value | 0.047 | 0.41 | 0.21 | 0.081 | 0.091 | 0.53 | 0.20 | 0.66 | 0.74 |
| 95% CI of | 1.0 | 0.37 | 0.69 | 0.90 | 0.74 | 0.54 | 0.61 | 0.24 | 0.34 |
| OR Quart 4 | 6.8 | 12 | 5.2 | 5.8 | 54 | 3.3 | 11 | 9.2 | 4.5 |

C-C motif chemokine 24

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 225 | 409 | 225 | 258 | 225 | 177 |
| Average | 418 | 443 | 418 | 391 | 418 | 265 |
| Stdev | 512 | 312 | 512 | 316 | 512 | 209 |
| p (t-test) | | 0.77 | | 0.75 | | 0.22 |
| Min | 0.0260 | 47.3 | 0.0260 | 54.7 | 0.0260 | 2.15 |
| Max | 2920 | 1470 | 2920 | 1380 | 2920 | 579 |
| n (Samp) | 93 | 45 | 93 | 42 | 93 | 18 |
| n (Patient) | 64 | 45 | 64 | 42 | 64 | 18 |
| sCr only | | | | | | |
| Median | 225 | 533 | 225 | 459 | 225 | 554 |
| Average | 357 | 535 | 357 | 456 | 357 | 615 |
| Stdev | 403 | 315 | 403 | 282 | 403 | 353 |
| p (t-test) | | 0.10 | | 0.31 | | 0.060 |
| Min | 0.0260 | 105 | 0.0260 | 94.6 | 0.0260 | 141 |
| Max | 2920 | 1260 | 2920 | 931 | 2920 | 1100 |
| n (Samp) | 224 | 14 | 224 | 18 | 224 | 9 |
| n (Patient) | 131 | 14 | 131 | 18 | 131 | 9 |
| UO only | | | | | | |
| Median | 270 | 409 | 270 | 259 | 270 | 126 |
| Average | 453 | 490 | 453 | 392 | 453 | 232 |
| Stdev | 493 | 461 | 493 | 344 | 493 | 212 |
| p (t-test) | | 0.69 | | 0.47 | | 0.091 |
| Min | 24.7 | 47.3 | 24.7 | 38.1 | 24.7 | 2.15 |
| Max | 2920 | 2430 | 2920 | 1380 | 2920 | 637 |
| n (Samp) | 102 | 37 | 102 | 41 | 102 | 15 |
| n (Patient) | 63 | 37 | 63 | 41 | 63 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.61 | 0.72 | 0.55 | 0.55 | 0.65 | 0.49 | 0.43 | 0.75 | 0.34 |
| SE | 0.052 | 0.079 | 0.056 | 0.054 | 0.073 | 0.054 | 0.076 | 0.096 | 0.081 |
| p | 0.042 | 0.0051 | 0.39 | 0.36 | 0.041 | 0.83 | 0.39 | 0.0091 | 0.042 |
| nCohort 1 | 93 | 224 | 102 | 93 | 224 | 102 | 93 | 224 | 102 |
| nCohort 2 | 45 | 14 | 37 | 42 | 18 | 41 | 18 | 9 | 15 |
| Cutoff 1 | 221 | 350 | 188 | 171 | 207 | 155 | 96.1 | 400 | 85.2 |
| Sens 1 | 71% | 71% | 70% | 71% | 72% | 71% | 72% | 78% | 73% |
| Spec 1 | 49% | 69% | 30% | 37% | 47% | 25% | 24% | 73% | 13% |
| Cutoff 2 | 171 | 221 | 154 | 108 | 196 | 92.4 | 85.2 | 212 | 70.8 |
| Sens 2 | 80% | 86% | 81% | 81% | 83% | 83% | 83% | 89% | 80% |
| Spec 2 | 37% | 50% | 25% | 25% | 46% | 15% | 18% | 47% | 11% |
| Cutoff 3 | 122 | 207 | 92.4 | 90.5 | 108 | 80.9 | 2.15 | 140 | 2.15 |
| Sens 3 | 91% | 93% | 92% | 90% | 94% | 90% | 94% | 100% | 93% |
| Spec 3 | 26% | 47% | 15% | 19% | 27% | 12% | 1% | 31% | 0% |
| Cutoff 4 | 393 | 365 | 541 | 393 | 365 | 541 | 393 | 365 | 541 |
| Sens 4 | 51% | 64% | 38% | 36% | 56% | 24% | 33% | 78% | 13% |
| Spec 4 | 71% | 70% | 71% | 71% | 70% | 71% | 71% | 70% | 71% |
| Cutoff 5 | 664 | 548 | 705 | 664 | 548 | 705 | 664 | 548 | 705 |
| Sens 5 | 18% | 50% | 22% | 21% | 39% | 17% | 0% | 56% | 0% |
| Spec 5 | 81% | 80% | 80% | 81% | 80% | 80% | 81% | 80% | 80% |
| Cutoff 6 | 940 | 855 | 993 | 940 | 855 | 993 | 940 | 855 | 993 |
| Sens 6 | 7% | 14% | 8% | 7% | 6% | 10% | 0% | 33% | 0% |
| Spec 6 | 90% | 90% | 90% | 90% | 90% | 90% | 90% | 90% | 90% |
| OR Quart 2 | 2.1 | >3.1 | 0.96 | 0.96 | 5.3 | 1.3 | 1.8 | >2.1 | 6.0 |
| p Value | 0.19 | <0.33 | 0.95 | 0.94 | 0.13 | 0.60 | 0.45 | <0.56 | 0.11 |
| 95% CI of | 0.69 | >0.31 | 0.31 | 0.33 | 0.60 | 0.47 | 0.39 | >0.18 | 0.66 |
| OR Quart 2 | 6.7 | na | 2.9 | 2.8 | 47 | 3.7 | 8.4 | na | 55 |
| OR Quart 3 | 3.7 | >3.2 | 1.5 | 1.7 | 3.1 | 1.3 | 1.8 | >1.0 | 0 |
| p Value | 0.021 | <0.33 | 0.46 | 0.34 | 0.33 | 0.60 | 0.45 | <0.99 | na |
| 95% CI of | 1.2 | >0.32 | 0.51 | 0.59 | 0.31 | 0.47 | 0.39 | >0.062 | na |
| OR Quart 3 | 11 | na | 4.3 | 4.6 | 31 | 3.7 | 8.4 | na | na |
| OR Quart 4 | 2.8 | >9.1 | 1.3 | 1.3 | 10 | 1.2 | 1.9 | >6.6 | 13 |
| p Value | 0.075 | <0.041 | 0.63 | 0.65 | 0.030 | 0.73 | 0.42 | <0.086 | 0.019 |
| 95% CI of | 0.90 | >1.1 | 0.44 | 0.45 | 1.3 | 0.42 | 0.41 | >0.77 | 1.5 |
| OR Quart 4 | 8.4 | na | 3.8 | 3.6 | 83 | 3.4 | 8.9 | na | 110 |

C-C motif chemokine 8

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 12.8 | 12.4 | 12.8 | 13.6 | 12.8 | 16.9 |
| Average | 17.4 | 13.3 | 17.4 | 18.7 | 17.4 | 20.9 |
| Stdev | 23.1 | 9.56 | 23.1 | 26.9 | 23.1 | 17.7 |
| p (t-test) | | 0.24 | | 0.78 | | 0.55 |
| Min | 0.162 | 0.162 | 0.162 | 0.170 | 0.162 | 1.32 |
| Max | 148 | 50.3 | 148 | 180 | 148 | 72.1 |
| n (Samp) | 93 | 45 | 93 | 42 | 93 | 18 |
| n (Patient) | 64 | 45 | 64 | 42 | 64 | 18 |
| sCr only | | | | | | |
| Median | 13.2 | 11.3 | 13.2 | 12.7 | 13.2 | 18.1 |
| Average | 16.7 | 12.7 | 16.7 | 22.4 | 16.7 | 23.8 |
| Stdev | 20.5 | 10.7 | 20.5 | 37.7 | 20.5 | 15.3 |
| p (t-test) | | 0.47 | | 0.30 | | 0.30 |
| Min | 0.162 | 0.162 | 0.162 | 0.170 | 0.162 | 11.5 |
| Max | 180 | 38.1 | 180 | 169 | 180 | 59.4 |
| n (Samp) | 224 | 14 | 224 | 18 | 224 | 9 |
| n (Patient) | 131 | 14 | 131 | 18 | 131 | 9 |
| UO only | | | | | | |
| Median | 12.3 | 12.4 | 12.3 | 13.8 | 12.3 | 16.3 |
| Average | 17.6 | 12.7 | 17.6 | 24.9 | 17.6 | 16.9 |
| Stdev | 22.8 | 9.01 | 22.8 | 40.3 | 22.8 | 16.3 |
| p (t-test) | | 0.20 | | 0.17 | | 0.90 |
| Min | 0.162 | 0.170 | 0.162 | 0.162 | 0.162 | 0.162 |
| Max | 148 | 50.3 | 148 | 180 | 148 | 72.1 |
| n (Samp) | 102 | 37 | 102 | 41 | 102 | 15 |
| n (Patient) | 63 | 37 | 63 | 41 | 63 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.46 | 0.43 | 0.47 | 0.54 | 0.50 | 0.55 | 0.61 | 0.71 | 0.55 |
| SE | 0.053 | 0.082 | 0.056 | 0.054 | 0.071 | 0.054 | 0.076 | 0.098 | 0.082 |
| p | 0.51 | 0.41 | 0.57 | 0.52 | 0.98 | 0.37 | 0.14 | 0.029 | 0.55 |
| nCohort 1 | 93 | 224 | 102 | 93 | 224 | 102 | 93 | 224 | 102 |
| nCohort 2 | 45 | 14 | 37 | 42 | 18 | 41 | 18 | 9 | 15 |
| Cutoff 1 | 7.62 | 6.24 | 8.03 | 10.1 | 10.6 | 9.32 | 12.9 | 14.4 | 11.3 |
| Sens 1 | 71% | 71% | 70% | 71% | 72% | 71% | 72% | 78% | 73% |
| Spec 1 | 25% | 19% | 28% | 37% | 36% | 30% | 53% | 57% | 44% |
| Cutoff 2 | 6.24 | 1.20 | 6.24 | 7.60 | 6.24 | 6.24 | 11.3 | 11.5 | 9.54 |
| Sens 2 | 82% | 86% | 81% | 81% | 83% | 83% | 83% | 89% | 80% |
| Spec 2 | 19% | 7% | 20% | 25% | 19% | 20% | 39% | 38% | 33% |
| Cutoff 3 | 1.16 | 1.16 | 1.16 | 4.81 | 1.67 | 4.81 | 9.47 | 11.3 | 1.20 |
| Sens 3 | 93% | 93% | 95% | 90% | 94% | 90% | 94% | 100% | 93% |
| Spec 3 | 6% | 5% | 8% | 18% | 9% | 19% | 29% | 38% | 10% |
| Cutoff 4 | 17.2 | 16.9 | 17.2 | 17.2 | 16.9 | 17.2 | 17.2 | 16.9 | 17.2 |
| Sens 4 | 18% | 29% | 19% | 36% | 33% | 34% | 39% | 67% | 33% |
| Spec 4 | 71% | 70% | 71% | 71% | 70% | 71% | 71% | 70% | 71% |
| Cutoff 5 | 20.1 | 19.7 | 22.1 | 20.1 | 19.7 | 22.1 | 20.1 | 19.7 | 22.1 |
| Sens 5 | 11% | 14% | 8% | 24% | 28% | 22% | 17% | 44% | 7% |
| Spec 5 | 81% | 80% | 80% | 81% | 80% | 80% | 81% | 80% | 80% |
| Cutoff 6 | 28.4 | 26.8 | 31.0 | 28.4 | 26.8 | 31.0 | 28.4 | 26.8 | 31.0 |
| Sens 6 | 4% | 14% | 3% | 10% | 17% | 12% | 17% | 22% | 7% |
| Spec 6 | 90% | 90% | 90% | 90% | 90% | 90% | 90% | 90% | 90% |
| OR Quart 2 | 2.4 | 1.0 | 2.9 | 0.96 | 0.98 | 0.60 | 4.3 | >2.1 | 1.6 |
| p Value | 0.11 | 0.98 | 0.065 | 0.94 | 0.98 | 0.37 | 0.20 | <0.56 | 0.64 |
| 95% CI of | 0.83 | 0.20 | 0.94 | 0.34 | 0.27 | 0.20 | 0.45 | >0.18 | 0.24 |
| OR Quart 2 | 6.7 | 5.3 | 8.7 | 2.7 | 3.6 | 1.8 | 42 | na | 10 |
| OR Quart 3 | 1.5 | 1.0 | 1.7 | 0.96 | 0.58 | 1.4 | 14 | >3.2 | 6.1 |
| p Value | 0.42 | 1.0 | 0.38 | 0.94 | 0.47 | 0.50 | 0.015 | <0.32 | 0.031 |
| 95% CI of | 0.53 | 0.19 | 0.52 | 0.34 | 0.13 | 0.52 | 1.7 | >0.32 | 1.2 |
| OR Quart 3 | 4.5 | 5.2 | 5.3 | 2.7 | 2.5 | 3.8 | 120 | na | 31 |
| OR Quart 4 | 1.8 | 1.8 | 1.7 | 1.3 | 0.98 | 1.1 | 3.1 | >4.2 | 0.47 |
| p Value | 0.26 | 0.45 | 0.35 | 0.66 | 0.98 | 0.85 | 0.34 | <0.20 | 0.54 |
| 95% CI of | 0.64 | 0.40 | 0.54 | 0.45 | 0.27 | 0.40 | 0.30 | >0.46 | 0.040 |
| OR Quart 4 | 5.3 | 7.7 | 5.6 | 3.5 | 3.6 | 3.1 | 32 | na | 5.4 |

Cathepsin D

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 234000 | 264000 | 234000 | 249000 | 234000 | 239000 |
| Average | 255000 | 257000 | 255000 | 278000 | 255000 | 272000 |
| Stdev | 136000 | 138000 | 136000 | 149000 | 136000 | 179000 |
| p (t-test) | | 0.93 | | 0.32 | | 0.59 |
| Min | 34100 | 55100 | 34100 | 55100 | 34100 | 54000 |
| Max | 1020000 | 653000 | 1020000 | 648000 | 1020000 | 853000 |
| n (Samp) | 120 | 53 | 120 | 53 | 120 | 26 |
| n (Patient) | 86 | 53 | 86 | 53 | 86 | 26 |
| sCr only | | | | | | |
| Median | 231000 | 210000 | 231000 | 325000 | 231000 | 175000 |
| Average | 266000 | 222000 | 266000 | 302000 | 266000 | 221000 |
| Stdev | 175000 | 110000 | 175000 | 145000 | 175000 | 99200 |
| p (t-test) | | 0.32 | | 0.44 | | 0.41 |
| Min | 34100 | 55100 | 34100 | 78800 | 34100 | 129000 |
| Max | 1870000 | 413000 | 1870000 | 507000 | 1870000 | 437000 |
| n (Samp) | 287 | 16 | 287 | 15 | 287 | 10 |
| n (Patient) | 160 | 16 | 160 | 15 | 160 | 10 |
| UO only | | | | | | |
| Median | 239000 | 280000 | 239000 | 252000 | 239000 | 248000 |
| Average | 265000 | 296000 | 265000 | 295000 | 265000 | 291000 |
| Stdev | 136000 | 216000 | 136000 | 164000 | 136000 | 186000 |
| p (t-test) | | 0.27 | | 0.21 | | 0.42 |
| Min | 69400 | 62900 | 69400 | 55100 | 69400 | 54000 |
| Max | 1020000 | 1350000 | 1020000 | 655000 | 1020000 | 853000 |
| n (Samp) | 124 | 43 | 124 | 54 | 124 | 23 |
| n (Patient) | 79 | 43 | 79 | 54 | 79 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.50 | 0.44 | 0.52 | 0.53 | 0.60 | 0.54 | 0.50 | 0.44 | 0.52 |
| SE | 0.048 | 0.076 | 0.052 | 0.048 | 0.079 | 0.047 | 0.063 | 0.096 | 0.066 |
| p | 0.94 | 0.41 | 0.64 | 0.47 | 0.23 | 0.42 | 0.98 | 0.53 | 0.77 |
| nCohort 1 | 120 | 287 | 124 | 120 | 287 | 124 | 120 | 287 | 124 |
| nCohort 2 | 53 | 16 | 43 | 53 | 15 | 54 | 26 | 10 | 23 |
| Cutoff 1 | 143000 | 143000 | 167000 | 167000 | 185000 | 169000 | 161000 | 157000 | 169000 |
| Sens 1 | 72% | 75% | 72% | 72% | 73% | 70% | 73% | 70% | 74% |
| Spec 1 | 18% | 23% | 28% | 29% | 38% | 28% | 25% | 28% | 29% |
| Cutoff 2 | 125000 | 126000 | 126000 | 136000 | 167000 | 136000 | 154000 | 154000 | 161000 |
| Sens 2 | 81% | 81% | 81% | 81% | 80% | 81% | 81% | 80% | 83% |
| Spec 2 | 11% | 15% | 7% | 14% | 33% | 10% | 21% | 27% | 23% |
| Cutoff 3 | 93800 | 87200 | 105000 | 116000 | 130000 | 114000 | 107000 | 130000 | 107000 |
| Sens 3 | 91% | 94% | 91% | 91% | 93% | 91% | 92% | 90% | 91% |
| Spec 3 | 6% | 4% | 6% | 8% | 17% | 6% | 7% | 17% | 6% |
| Cutoff 4 | 300000 | 307000 | 307000 | 300000 | 307000 | 307000 | 300000 | 307000 | 307000 |
| Sens 4 | 36% | 25% | 40% | 42% | 53% | 41% | 27% | 20% | 30% |
| Spec 4 | 70% | 70% | 70% | 70% | 70% | 70% | 70% | 70% | 70% |
| Cutoff 5 | 330000 | 362000 | 342000 | 330000 | 362000 | 342000 | 330000 | 362000 | 342000 |
| Sens 5 | 32% | 12% | 26% | 36% | 33% | 39% | 19% | 10% | 26% |
| Spec 5 | 80% | 80% | 81% | 80% | 80% | 81% | 80% | 80% | 81% |
| Cutoff 6 | 407000 | 462000 | 426000 | 407000 | 462000 | 426000 | 407000 | 462000 | 426000 |
| Sens 6 | 15% | 0% | 14% | 25% | 20% | 22% | 15% | 0% | 17% |
| Spec 6 | 90% | 90% | 90% | 90% | 90% | 90% | 90% | 90% | 90% |
| OR Quart 2 | 0.35 | 0.74 | 0.40 | 0.57 | 0.99 | 0.42 | 1.5 | 3.1 | 0.97 |
| p Value | 0.036 | 0.70 | 0.10 | 0.24 | 0.99 | 0.083 | 0.52 | 0.33 | 0.96 |
| 95% CI of | 0.13 | 0.16 | 0.13 | 0.22 | 0.19 | 0.16 | 0.46 | 0.32 | 0.26 |
| OR Quart 2 | 0.93 | 3.4 | 1.2 | 1.5 | 5.0 | 1.1 | 4.8 | 31 | 3.7 |
| OR Quart 3 | 0.46 | 1.0 | 0.86 | 0.49 | 1.0 | 0.57 | 0.81 | 4.2 | 1.4 |
| p Value | 0.11 | 1.0 | 0.75 | 0.15 | 1.0 | 0.24 | 0.74 | 0.20 | 0.56 |
| 95% CI of | 0.18 | 0.24 | 0.33 | 0.19 | 0.20 | 0.22 | 0.22 | 0.46 | 0.41 |
| OR Quart 3 | 1.2 | 4.2 | 2.2 | 1.3 | 5.1 | 1.5 | 2.9 | 39 | 5.1 |
| OR Quart 4 | 1.1 | 1.3 | 1.2 | 1.4 | 2.1 | 1.7 | 1.2 | 2.1 | 1.2 |
| p Value | 0.90 | 0.72 | 0.69 | 0.43 | 0.32 | 0.23 | 0.72 | 0.56 | 0.78 |
| 95% CI of | 0.45 | 0.33 | 0.48 | 0.60 | 0.50 | 0.72 | 0.37 | 0.18 | 0.33 |
| OR Quart 4 | 2.5 | 5.0 | 3.1 | 3.4 | 8.5 | 4.0 | 4.2 | 23 | 4.3 |

C—X—C motif chemokine 13

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 45.8 | 52.8 | 45.8 | 69.6 | 45.8 | 80.6 |
| Average | 138 | 116 | 138 | 118 | 138 | 224 |
| Stdev | 297 | 292 | 297 | 216 | 297 | 275 |
| p (t-test) | | 0.68 | | 0.69 | | 0.26 |
| Min | 9.90 | 17.0 | 9.90 | 11.3 | 9.90 | 15.9 |
| Max | 1790 | 2000 | 1790 | 1340 | 1790 | 918 |
| n (Samp) | 93 | 45 | 93 | 42 | 93 | 18 |
| n (Patient) | 64 | 45 | 64 | 42 | 64 | 18 |
| sCr only | | | | | | |
| Median | 48.7 | 62.3 | 48.7 | 69.5 | 48.7 | 43.9 |
| Average | 125 | 207 | 125 | 187 | 125 | 128 |
| Stdev | 242 | 517 | 242 | 455 | 242 | 238 |
| p (t-test) | | 0.26 | | 0.33 | | 0.96 |
| Min | 9.90 | 25.4 | 9.90 | 18.6 | 9.90 | 17.3 |
| Max | 1790 | 2000 | 1790 | 2000 | 1790 | 758 |
| n (Samp) | 224 | 14 | 224 | 18 | 224 | 9 |
| n (Patient) | 131 | 14 | 131 | 18 | 131 | 9 |
| UO only | | | | | | |
| Median | 47.3 | 56.6 | 47.3 | 69.3 | 47.3 | 81.6 |
| Average | 123 | 76.0 | 123 | 170 | 123 | 229 |
| Stdev | 242 | 57.1 | 242 | 285 | 242 | 257 |
| p (t-test) | | 0.25 | | 0.32 | | 0.12 |
| Min | 9.90 | 17.0 | 9.90 | 11.3 | 9.90 | 15.9 |
| Max | 1790 | 287 | 1790 | 1340 | 1790 | 918 |
| n (Samp) | 102 | 37 | 102 | 41 | 102 | 15 |
| n (Patient) | 63 | 37 | 63 | 41 | 63 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.54 | 0.57 | 0.52 | 0.55 | 0.58 | 0.58 | 0.63 | 0.50 | 0.64 |
| SE | 0.053 | 0.082 | 0.056 | 0.054 | 0.073 | 0.054 | 0.076 | 0.099 | 0.081 |
| p | 0.47 | 0.42 | 0.66 | 0.38 | 0.25 | 0.15 | 0.096 | 0.97 | 0.082 |
| nCohort 1 | 93 | 224 | 102 | 93 | 224 | 102 | 93 | 224 | 102 |
| nCohort 2 | 45 | 14 | 37 | 42 | 18 | 41 | 18 | 9 | 15 |
| Cutoff 1 | 37.0 | 46.6 | 37.3 | 33.7 | 52.7 | 39.4 | 41.8 | 34.7 | 47.3 |
| Sens 1 | 71% | 71% | 70% | 71% | 72% | 71% | 72% | 78% | 73% |
| Spec 1 | 44% | 48% | 41% | 42% | 53% | 41% | 46% | 37% | 52% |
| Cutoff 2 | 34.0 | 28.0 | 34.5 | 28.3 | 32.8 | 33.7 | 29.5 | 34.0 | 29.5 |
| Sens 2 | 80% | 86% | 81% | 81% | 83% | 80% | 83% | 89% | 80% |
| Spec 2 | 42% | 27% | 35% | 35% | 35% | 34% | 41% | 36% | 33% |
| Cutoff 3 | 23.0 | 25.8 | 18.7 | 20.5 | 24.4 | 22.7 | 16.8 | 17.2 | 16.8 |
| Sens 3 | 91% | 93% | 92% | 90% | 94% | 90% | 94% | 100% | 93% |
| Spec 3 | 26% | 24% | 12% | 17% | 21% | 20% | 12% | 12% | 11% |
| Cutoff 4 | 106 | 98.9 | 102 | 106 | 98.9 | 102 | 106 | 98.9 | 102 |
| Sens 4 | 24% | 29% | 24% | 33% | 33% | 39% | 39% | 22% | 47% |
| Spec 4 | 71% | 70% | 71% | 71% | 70% | 71% | 71% | 70% | 71% |
| Cutoff 5 | 170 | 136 | 140 | 170 | 136 | 140 | 170 | 136 | 140 |
| Sens 5 | 9% | 14% | 11% | 7% | 22% | 22% | 39% | 11% | 47% |
| Spec 5 | 81% | 80% | 80% | 81% | 80% | 80% | 81% | 80% | 80% |
| Cutoff 6 | 251 | 254 | 232 | 251 | 254 | 232 | 251 | 254 | 232 |
| Sens 6 | 4% | 7% | 3% | 7% | 6% | 15% | 39% | 11% | 47% |
| Spec 6 | 90% | 90% | 90% | 90% | 90% | 90% | 90% | 90% | 90% |
| OR Quart 2 | 2.3 | 1.5 | 2.7 | 1.5 | 1.5 | 1.3 | 0.96 | 2.1 | 0.64 |
| p Value | 0.13 | 0.66 | 0.11 | 0.44 | 0.66 | 0.61 | 0.96 | 0.56 | 0.64 |
| 95% CI of | 0.78 | 0.24 | 0.81 | 0.51 | 0.24 | 0.43 | 0.18 | 0.18 | 0.099 |
| OR Quart 2 | 6.7 | 9.3 | 8.7 | 4.7 | 9.3 | 4.1 | 5.2 | 23 | 4.2 |
| OR Quart 3 | 3.4 | 3.2 | 3.9 | 2.9 | 4.5 | 2.0 | 1.7 | 5.5 | 1.0 |
| p Value | 0.024 | 0.16 | 0.023 | 0.050 | 0.066 | 0.21 | 0.48 | 0.13 | 1.0 |
| 95% CI of | 1.2 | 0.62 | 1.2 | 1.0 | 0.91 | 0.68 | 0.37 | 0.62 | 0.18 |
| OR Quart 3 | 10.0 | 17 | 12 | 8.6 | 22 | 5.9 | 8.1 | 48 | 5.4 |
| OR Quart 4 | 1.3 | 1.5 | 1.4 | 1.5 | 2.6 | 2.3 | 2.7 | 1.0 | 2.6 |
| p Value | 0.61 | 0.66 | 0.56 | 0.44 | 0.27 | 0.14 | 0.19 | 0.99 | 0.19 |
| 95% CI of | 0.43 | 0.24 | 0.41 | 0.51 | 0.48 | 0.77 | 0.61 | 0.062 | 0.61 |
| OR Quart 4 | 4.1 | 9.3 | 5.1 | 4.7 | 14 | 6.6 | 12 | 17 | 11 |

Insulin-like growth factor-binding protein 3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3010 | 3320 | 3010 | 3210 | 3010 | 3990 |
| Average | 3360 | 3500 | 3360 | 3620 | 3360 | 4050 |
| Stdev | 1520 | 1470 | 1520 | 1710 | 1520 | 1940 |
| p (t-test) | | 0.58 | | 0.33 | | 0.050 |
| Min | 823 | 651 | 823 | 730 | 823 | 804 |
| Max | 8260 | 7620 | 8260 | 8600 | 8260 | 7140 |
| n (Samp) | 121 | 53 | 121 | 53 | 121 | 26 |
| n (Patient) | 87 | 53 | 87 | 53 | 87 | 26 |
| sCr only | | | | | | |
| Median | 3050 | 3580 | 3050 | 4790 | 3050 | 5000 |
| Average | 3390 | 4110 | 3390 | 4440 | 3390 | 4510 |
| Stdev | 1500 | 1990 | 1500 | 1990 | 1500 | 1960 |
| p (t-test) | | 0.065 | | 0.0096 | | 0.022 |
| Min | 651 | 1820 | 651 | 1210 | 651 | 1660 |
| Max | 8260 | 7620 | 8260 | 8600 | 8260 | 7140 |
| n (Samp) | 288 | 16 | 288 | 15 | 288 | 10 |
| n (Patient) | 161 | 16 | 161 | 15 | 161 | 10 |
| UO only | | | | | | |
| Median | 2960 | 3320 | 2960 | 3220 | 2960 | 3300 |
| Average | 3290 | 3420 | 3290 | 3520 | 3290 | 3630 |
| Stdev | 1510 | 1370 | 1510 | 1580 | 1510 | 1780 |
| p (t-test) | | 0.61 | | 0.36 | | 0.34 |
| Min | 823 | 651 | 823 | 730 | 823 | 804 |
| Max | 8260 | 7300 | 8260 | 7520 | 8260 | 7100 |
| n (Samp) | 125 | 43 | 125 | 54 | 125 | 23 |
| n (Patient) | 80 | 43 | 80 | 54 | 80 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.54 | 0.59 | 0.55 | 0.54 | 0.66 | 0.55 | 0.61 | 0.67 | 0.56 |
| SE | 0.048 | 0.077 | 0.052 | 0.048 | 0.078 | 0.047 | 0.064 | 0.095 | 0.067 |
| p | 0.42 | 0.23 | 0.32 | 0.41 | 0.039 | 0.33 | 0.090 | 0.068 | 0.41 |
| nCohort 1 | 121 | 288 | 125 | 121 | 288 | 125 | 121 | 288 | 125 |
| nCohort 2 | 53 | 16 | 43 | 53 | 15 | 54 | 26 | 10 | 23 |
| Cutoff 1 | 2620 | 2560 | 2670 | 2370 | 3020 | 2430 | 2700 | 3050 | 2430 |
| Sens 1 | 72% | 75% | 72% | 72% | 73% | 70% | 73% | 70% | 74% |
| Spec 1 | 40% | 36% | 44% | 30% | 50% | 34% | 44% | 50% | 34% |
| Cutoff 2 | 2160 | 2070 | 2160 | 2290 | 2800 | 2290 | 2040 | 2430 | 2000 |
| Sens 2 | 81% | 81% | 81% | 81% | 80% | 81% | 81% | 80% | 83% |
| Spec 2 | 26% | 19% | 28% | 27% | 43% | 30% | 23% | 31% | 22% |
| Cutoff 3 | 1960 | 1940 | 1740 | 1780 | 2130 | 1740 | 1490 | 2430 | 1490 |
| Sens 3 | 91% | 94% | 91% | 91% | 93% | 91% | 92% | 90% | 91% |
| Spec 3 | 20% | 15% | 11% | 12% | 20% | 11% | 7% | 31% | 7% |
| Cutoff 4 | 4130 | 4130 | 4040 | 4130 | 4130 | 4040 | 4130 | 4130 | 4040 |
| Sens 4 | 28% | 44% | 33% | 34% | 53% | 37% | 50% | 60% | 39% |
| Spec 4 | 70% | 70% | 70% | 70% | 70% | 70% | 70% | 70% | 70% |
| Cutoff 5 | 4680 | 4650 | 4560 | 4680 | 4650 | 4560 | 4680 | 4650 | 4560 |
| Sens 5 | 17% | 38% | 16% | 26% | 53% | 26% | 38% | 60% | 30% |
| Spec 5 | 80% | 80% | 80% | 80% | 80% | 80% | 80% | 80% | 80% |
| Cutoff 6 | 5410 | 5490 | 5410 | 5410 | 5490 | 5410 | 5410 | 5490 | 5410 |
| Sens 6 | 8% | 31% | 5% | 19% | 40% | 15% | 31% | 40% | 22% |
| Spec 6 | 90% | 90% | 90% | 90% | 90% | 90% | 90% | 90% | 90% |
| OR Quart 2 | 0.86 | 0.74 | 1.6 | 1.5 | 0.65 | 2.1 | 0.44 | 3.0 | 0.63 |
| p Value | 0.76 | 0.70 | 0.42 | 0.37 | 0.64 | 0.12 | 0.28 | 0.34 | 0.50 |
| 95% CI of | 0.33 | 0.16 | 0.53 | 0.60 | 0.11 | 0.83 | 0.10 | 0.31 | 0.16 |
| OR Quart 2 | 2.2 | 3.4 | 4.6 | 4.0 | 4.0 | 5.6 | 1.9 | 30 | 2.4 |
| OR Quart 3 | 1.9 | 0.74 | 2.8 | 1.4 | 0.65 | 1.6 | 0.97 | 0 | 0.81 |
| p Value | 0.18 | 0.70 | 0.051 | 0.47 | 0.64 | 0.36 | 0.96 | na | 0.74 |
| 95% CI of | 0.76 | 0.16 | 0.99 | 0.55 | 0.11 | 0.60 | 0.28 | na | 0.22 |
| OR Quart 3 | 4.6 | 3.4 | 7.8 | 3.7 | 4.0 | 4.2 | 3.3 | na | 2.9 |
| OR Quart 4 | 0.97 | 1.5 | 1.8 | 1.9 | 2.8 | 2.1 | 2.1 | 6.3 | 1.4 |
| p Value | 0.95 | 0.52 | 0.29 | 0.18 | 0.14 | 0.12 | 0.19 | 0.091 | 0.55 |
| 95% CI of | 0.38 | 0.42 | 0.61 | 0.74 | 0.72 | 0.83 | 0.69 | 0.75 | 0.44 |
| OR Quart 4 | 2.5 | 5.7 | 5.1 | 4.8 | 11 | 5.6 | 6.5 | 54 | 4.6 |

Immunoglogulin G1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 8750000 | 8400000 | 8750000 | 9530000 | 8750000 | 7580000 |
| Average | 1.06E7 | 9010000 | 1.06E7 | 1.24E7 | 1.06E7 | 1.30E7 |
| Stdev | 6040000 | 4090000 | 6040000 | 8340000 | 6040000 | 9920000 |
| p (t-test) | | 0.14 | | 0.19 | | 0.28 |
| Min | 3800000 | 2060000 | 3800000 | 3530000 | 3800000 | 4390000 |
| Max | 3.48E7 | 2.33E7 | 3.48E7 | 4.18E7 | 3.48E7 | 3.57E7 |
| n (Samp) | 94 | 35 | 94 | 37 | 94 | 10 |
| n (Patient) | 67 | 35 | 67 | 37 | 67 | 10 |
| sCr only | | | | | | |
| Median | 8820000 | 7580000 | 8820000 | 1.02E7 | nd | nd |
| Average | 1.02E7 | 9650000 | 1.02E7 | 1.51E7 | nd | nd |
| Stdev | 5310000 | 6060000 | 5310000 | 1.34E7 | nd | nd |
| p (t-test) | | 0.73 | | 0.021 | nd | nd |
| Min | 2060000 | 3270000 | 2060000 | 4390000 | nd | nd |
| Max | 3.48E7 | 2.33E7 | 3.48E7 | 4.18E7 | nd | nd |
| n (Samp) | 205 | 10 | 205 | 8 | nd | nd |
| n (Patient) | 126 | 10 | 126 | 8 | nd | nd |
| UO only | | | | | | |
| Median | 7990000 | 8420000 | 7990000 | 9200000 | 7990000 | 7690000 |
| Average | 9560000 | 8580000 | 9560000 | 1.21E7 | 9560000 | 1.34E7 |
| Stdev | 5620000 | 3160000 | 5620000 | 7850000 | 5620000 | 9630000 |
| p (t-test) | | 0.38 | | 0.039 | | 0.043 |
| Min | 3270000 | 2060000 | 3270000 | 3530000 | 3270000 | 4390000 |
| Max | 3.48E7 | 1.63E7 | 3.48E7 | 4.18E7 | 3.48E7 | 3.57E7 |
| n (Samp) | 101 | 28 | 101 | 36 | 101 | 12 |
| n (Patient) | 65 | 28 | 65 | 36 | 65 | 12 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.45 | 0.42 | 0.51 | 0.55 | 0.56 | 0.61 | 0.54 | nd | 0.62 |
| SE | 0.058 | 0.097 | 0.062 | 0.057 | 0.11 | 0.056 | 0.098 | nd | 0.091 |
| p | 0.38 | 0.42 | 0.82 | 0.41 | 0.55 | 0.041 | 0.69 | nd | 0.18 |
| nCohort 1 | 94 | 205 | 101 | 94 | 205 | 101 | 94 | nd | 101 |
| nCohort 2 | 35 | 10 | 28 | 37 | 8 | 36 | 10 | nd | 12 |
| Cutoff 1 | 6710000 | 6260000 | 7160000 | 7600000 | 7160000 | 7600000 | 7140000 | nd | 7140000 |
| Sens 1 | 71% | 70% | 71% | 70% | 75% | 72% | 90% | nd | 92% |
| Spec 1 | 27% | 17% | 44% | 40% | 33% | 50% | 31% | nd | 41% |
| Cutoff 2 | 6260000 | 5810000 | 6300000 | 6740000 | 5340000 | 6940000 | 7140000 | nd | 7140000 |
| Sens 2 | 80% | 80% | 82% | 81% | 88% | 81% | 90% | nd | 92% |
| Spec 2 | 20% | 15% | 34% | 29% | 12% | 38% | 31% | nd | 41% |
| Cutoff 3 | 5320000 | 5320000 | 5320000 | 5340000 | 4360000 | 5370000 | 7140000 | nd | 7140000 |
| Sens 3 | 94% | 90% | 93% | 92% | 100% | 92% | 90% | nd | 92% |
| Spec 3 | 10% | 8% | 13% | 14% | 4% | 21% | 31% | nd | 41% |
| Cutoff 4 | 1.13E7 | 1.11E7 | 1.05E7 | 1.13E7 | 1.11E7 | 1.05E7 | 1.13E7 | nd | 1.05E7 |
| Sens 4 | 23% | 30% | 25% | 32% | 38% | 36% | 40% | nd | 42% |
| Spec 4 | 70% | 70% | 70% | 70% | 70% | 70% | 70% | nd | 70% |
| Cutoff 5 | 1.43E7 | 1.34E7 | 1.17E7 | 1.43E7 | 1.34E7 | 1.17E7 | 1.43E7 | nd | 1.17E7 |
| Sens 5 | 9% | 20% | 14% | 30% | 25% | 36% | 30% | nd | 42% |
| Spec 5 | 81% | 80% | 80% | 81% | 80% | 80% | 81% | nd | 80% |
| Cutoff 6 | 1.98E7 | 1.71E7 | 1.59E7 | 1.98E7 | 1.71E7 | 1.59E7 | 1.98E7 | nd | 1.59E7 |
| Sens 6 | 3% | 10% | 4% | 16% | 25% | 19% | 20% | nd | 25% |
| Spec 6 | 90% | 90% | 90% | 90% | 90% | 90% | 90% | nd | 90% |
| OR Quart 2 | 1.2 | 0 | 1.5 | 2.2 | 0.49 | 2.7 | 6.0 | nd | 5.9 |
| p Value | 0.72 | na | 0.52 | 0.19 | 0.57 | 0.13 | 0.12 | nd | 0.12 |
| 95% CI of | 0.39 | na | 0.42 | 0.69 | 0.043 | 0.74 | 0.64 | nd | 0.64 |
| OR Quart 2 | 3.9 | na | 5.4 | 6.8 | 5.6 | 9.8 | 55 | nd | 54 |
| OR Quart 3 | 1.9 | 1.0 | 2.5 | 1.6 | 1.5 | 3.1 | 0 | nd | 1.0 |
| p Value | 0.24 | 1.0 | 0.15 | 0.42 | 0.65 | 0.080 | na | nd | 1.0 |
| 95% CI of | 0.64 | 0.19 | 0.73 | 0.50 | 0.25 | 0.87 | na | nd | 0.059 |
| OR Quart 3 | 5.9 | 5.2 | 8.2 | 5.2 | 9.5 | 11 | na | nd | 17 |
| OR Quart 4 | 1.5 | 1.4 | 1.2 | 2.2 | 0.98 | 4.4 | 4.5 | nd | 5.6 |
| p Value | 0.52 | 0.68 | 0.78 | 0.19 | 0.98 | 0.019 | 0.19 | nd | 0.13 |
| 95% CI of | 0.47 | 0.30 | 0.33 | 0.69 | 0.13 | 1.3 | 0.47 | nd | 0.61 |
| OR Quart 4 | 4.5 | 6.5 | 4.4 | 6.8 | 7.2 | 15 | 44 | nd | 52 |

Immunoglogulin G2

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.41E7 | 1.03E7 | 1.41E7 | 1.08E7 | 1.41E7 | 1.08E7 |
| Average | 1.56E7 | 1.09E7 | 1.56E7 | 1.31E7 | 1.56E7 | 1.03E7 |
| Stdev | 1.10E7 | 8880000 | 1.10E7 | 6520000 | 1.10E7 | 6120000 |
| p (t-test) | | 0.027 | | 0.19 | | 0.14 |
| Min | 219000 | 190000 | 219000 | 1750000 | 219000 | 1750000 |
| Max | 5.80E7 | 4.70E7 | 5.80E7 | 3.11E7 | 5.80E7 | 1.77E7 |
| n (Samp) | 94 | 35 | 94 | 37 | 94 | 10 |
| n (Patient) | 67 | 35 | 67 | 37 | 67 | 10 |
| sCr only | | | | | | |
| Median | 1.03E7 | 1.21E7 | 1.03E7 | 1.34E7 | nd | nd |
| Average | 1.30E7 | 1.47E7 | 1.30E7 | 1.41E7 | nd | nd |
| Stdev | 9120000 | 1.27E7 | 9120000 | 9110000 | nd | nd |
| p (t-test) | | 0.57 | | 0.74 | nd | nd |
| Min | 190000 | 1750000 | 190000 | 1750000 | nd | nd |
| Max | 5.80E7 | 4.70E7 | 5.80E7 | 2.70E7 | nd | nd |
| n (Samp) | 205 | 10 | 205 | 8 | nd | nd |
| n (Patient) | 126 | 10 | 126 | 8 | nd | nd |
| UO only | | | | | | |
| Median | 1.03E7 | 1.03E7 | 1.03E7 | 1.08E7 | 1.03E7 | 1.08E7 |
| Average | 1.41E7 | 1.02E7 | 1.41E7 | 1.38E7 | 1.41E7 | 1.34E7 |
| Stdev | 1.02E7 | 7510000 | 1.02E7 | 7620000 | 1.02E7 | 1.20E7 |
| p (t-test) | | 0.060 | | 0.87 | | 0.82 |
| Min | 219000 | 190000 | 219000 | 1750000 | 219000 | 1750000 |
| Max | 5.80E7 | 2.91E7 | 5.80E7 | 3.69E7 | 5.80E7 | 4.70E7 |
| n (Samp) | 101 | 28 | 101 | 36 | 101 | 12 |
| n (Patient) | 65 | 28 | 65 | 36 | 65 | 12 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.38 | 0.52 | 0.41 | 0.47 | 0.56 | 0.53 | 0.39 | nd | 0.48 |
| SE | 0.057 | 0.095 | 0.063 | 0.057 | 0.11 | 0.057 | 0.099 | nd | 0.089 |
| p | 0.036 | 0.79 | 0.14 | 0.59 | 0.58 | 0.58 | 0.27 | nd | 0.79 |
| nCohort 1 | 94 | 205 | 101 | 94 | 205 | 101 | 94 | nd | 101 |
| nCohort 2 | 35 | 10 | 28 | 37 | 8 | 36 | 10 | nd | 12 |
| Cutoff 1 | 6810000 | 9110000 | 2450000 | 1.02E7 | 1.02E7 | 9110000 | 6700000 | nd | 6700000 |
| Sens 1 | 71% | 80% | 71% | 70% | 75% | 75% | 70% | nd | 75% |
| Spec 1 | 19% | 31% | 15% | 38% | 41% | 31% | 16% | nd | 20% |
| Cutoff 2 | 1750000 | 9110000 | 219000 | 7340000 | 4550000 | 7850000 | 4550000 | nd | 4550000 |
| Sens 2 | 80% | 80% | 96% | 84% | 88% | 83% | 80% | nd | 83% |
| Spec 2 | 9% | 31% | 1% | 21% | 18% | 26% | 14% | nd | 17% |
| Cutoff 3 | 563000 | 1930000 | 219000 | 4550000 | 563000 | 5450000 | 1750000 | nd | 1750000 |
| Sens 3 | 97% | 90% | 96% | 92% | 100% | 92% | 90% | nd | 92% |
| Spec 3 | 2% | 11% | 1% | 14% | 1% | 18% | 9% | nd | 8% |
| Cutoff 4 | 1.77E7 | 1.61E7 | 1.64E7 | 1.77E7 | 1.61E7 | 1.64E7 | 1.77E7 | nd | 1.64E7 |
| Sens 4 | 14% | 30% | 18% | 16% | 50% | 31% | 0% | nd | 33% |
| Spec 4 | 71% | 70% | 72% | 71% | 70% | 72% | 71% | nd | 72% |
| Cutoff 5 | 2.34E7 | 1.77E7 | 2.09E7 | 2.34E7 | 1.77E7 | 2.09E7 | 2.34E7 | nd | 2.09E7 |
| Sens 5 | 6% | 20% | 7% | 8% | 25% | 11% | 0% | nd | 8% |
| Spec 5 | 81% | 81% | 80% | 81% | 81% | 80% | 81% | nd | 80% |
| Cutoff 6 | 3.13E7 | 2.52E7 | 2.66E7 | 3.13E7 | 2.52E7 | 2.66E7 | 3.13E7 | nd | 2.66E7 |
| Sens 6 | 3% | 10% | 4% | 0% | 25% | 8% | 0% | nd | 8% |
| Spec 6 | 91% | 91% | 90% | 91% | 91% | 90% | 91% | nd | 90% |
| OR Quart 2 | 1.5 | 1.5 | 1.6 | 2.2 | 1.0 | 1.2 | >4.7 | nd | 2.9 |
| p Value | 0.51 | 0.66 | 0.49 | 0.16 | 1.0 | 0.77 | <0.18 | nd | 0.22 |
| 95% CI of | 0.46 | 0.24 | 0.44 | 0.72 | 0.14 | 0.38 | >0.49 | nd | 0.52 |
| OR Quart 2 | 4.9 | 9.4 | 5.6 | 7.1 | 7.4 | 3.7 | na | nd | 17 |
| OR Quart 3 | 2.0 | 0.98 | 1.6 | 2.6 | 0.49 | 2.1 | >2.2 | nd | 0.50 |
| p Value | 0.23 | 0.98 | 0.49 | 0.10 | 0.57 | 0.18 | <0.54 | nd | 0.58 |
| 95% CI of | 0.64 | 0.13 | 0.44 | 0.83 | 0.043 | 0.71 | >0.18 | nd | 0.043 |
| OR Quart 3 | 6.5 | 7.2 | 5.6 | 8.0 | 5.6 | 6.3 | na | nd | 5.8 |
| OR Quart 4 | 2.4 | 1.5 | 2.2 | 1.5 | 1.5 | 1.3 | >4.7 | nd | 2.2 |
| p Value | 0.14 | 0.66 | 0.21 | 0.51 | 0.66 | 0.61 | <0.18 | nd | 0.37 |
| 95% CI of | 0.75 | 0.24 | 0.64 | 0.46 | 0.24 | 0.43 | >0.49 | nd | 0.38 |
| OR Quart 4 | 7.4 | 9.4 | 7.5 | 4.9 | 9.4 | 4.1 | na | nd | 13 |

Interleukin-11

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 60.0 | 90.7 | 60.0 | 74.1 | 60.0 | 44.3 |
| Average | 245 | 134 | 245 | 115 | 245 | 90.2 |
| Stdev | 951 | 286 | 951 | 141 | 951 | 135 |
| p (t-test) | | 0.45 | | 0.38 | | 0.49 |
| Min | 0.368 | 0.359 | 0.368 | 0.359 | 0.368 | 0.359 |
| Max | 6920 | 1940 | 6920 | 802 | 6920 | 569 |
| n (Samp) | 94 | 45 | 94 | 42 | 94 | 18 |
| n (Patient) | 65 | 45 | 65 | 42 | 65 | 18 |
| sCr only | | | | | | |
| Median | 59.4 | 91.0 | 59.4 | 89.3 | 59.4 | 67.2 |
| Average | 148 | 236 | 148 | 465 | 148 | 118 |
| Stdev | 621 | 502 | 621 | 1250 | 621 | 171 |
| p (t-test) | | 0.60 | | 0.060 | | 0.89 |
| Min | 0.359 | 0.480 | 0.359 | 0.442 | 0.359 | 20.4 |
| Max | 6920 | 1940 | 6920 | 5420 | 6920 | 569 |
| n (Samp) | 225 | 14 | 225 | 18 | 225 | 9 |
| n (Patient) | 132 | 14 | 132 | 18 | 132 | 9 |
| UO only | | | | | | |
| Median | 47.4 | 71.6 | 47.4 | 83.4 | 47.4 | 38.4 |
| Average | 218 | 83.1 | 218 | 107 | 218 | 60.7 |
| Stdev | 909 | 62.4 | 909 | 125 | 909 | 69.0 |
| p (t-test) | | 0.37 | | 0.44 | | 0.51 |
| Min | 0.368 | 0.359 | 0.368 | 0.359 | 0.368 | 0.359 |
| Max | 6920 | 234 | 6920 | 741 | 6920 | 233 |
| n (Samp) | 103 | 37 | 103 | 41 | 103 | 15 |
| n (Patient) | 64 | 37 | 64 | 41 | 64 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.53 | 0.57 | 0.53 | 0.53 | 0.64 | 0.57 | 0.47 | 0.56 | 0.44 |
| SE | 0.053 | 0.082 | 0.056 | 0.054 | 0.073 | 0.054 | 0.075 | 0.10 | 0.082 |
| p | 0.56 | 0.39 | 0.57 | 0.53 | 0.048 | 0.22 | 0.73 | 0.56 | 0.50 |
| nCohort 1 | 94 | 225 | 103 | 94 | 225 | 103 | 94 | 225 | 103 |
| nCohort 2 | 45 | 14 | 37 | 42 | 18 | 41 | 18 | 9 | 15 |
| Cutoff 1 | 46.9 | 38.4 | 42.3 | 41.7 | 55.3 | 47.4 | 33.9 | 43.7 | 20.2 |
| Sens 1 | 71% | 71% | 70% | 71% | 72% | 71% | 72% | 78% | 73% |
| Spec 1 | 46% | 39% | 47% | 41% | 49% | 50% | 40% | 44% | 33% |
| Cutoff 2 | 29.7 | 6.00 | 15.1 | 15.1 | 29.7 | 20.2 | 20.2 | 38.4 | 7.90 |
| Sens 2 | 82% | 86% | 81% | 81% | 83% | 83% | 83% | 89% | 87% |
| Spec 2 | 34% | 16% | 26% | 23% | 31% | 33% | 31% | 39% | 24% |
| Cutoff 3 | 0.608 | 0.442 | 0.442 | 6.00 | 10.7 | 6.00 | 7.90 | 20.2 | 0.442 |
| Sens 3 | 91% | 100% | 92% | 90% | 94% | 90% | 94% | 100% | 93% |
| Spec 3 | 9% | 8% | 6% | 15% | 23% | 16% | 22% | 28% | 6% |
| Cutoff 4 | 139 | 113 | 118 | 139 | 113 | 118 | 139 | 113 | 118 |
| Sens 4 | 24% | 29% | 24% | 29% | 44% | 29% | 17% | 11% | 13% |
| Spec 4 | 70% | 71% | 71% | 70% | 71% | 71% | 70% | 71% | 71% |
| Cutoff 5 | 190 | 155 | 159 | 190 | 155 | 159 | 190 | 155 | 159 |
| Sens 5 | 9% | 29% | 14% | 19% | 44% | 17% | 17% | 11% | 13% |
| Spec 5 | 81% | 80% | 81% | 81% | 80% | 81% | 81% | 80% | 81% |
| Cutoff 6 | 270 | 209 | 221 | 270 | 209 | 221 | 270 | 209 | 221 |
| Sens 6 | 4% | 21% | 3% | 7% | 33% | 7% | 6% | 11% | 7% |
| Spec 6 | 90% | 91% | 90% | 90% | 91% | 90% | 90% | 91% | 90% |
| OR Quart 2 | 1.5 | 0.64 | 1.2 | 1.4 | 0.98 | 2.2 | 1.4 | >3.1 | 1.6 |
| p Value | 0.46 | 0.64 | 0.77 | 0.58 | 0.98 | 0.17 | 0.69 | <0.33 | 0.61 |
| 95% CI of | 0.51 | 0.10 | 0.38 | 0.46 | 0.19 | 0.71 | 0.28 | >0.31 | 0.25 |
| OR Quart 2 | 4.3 | 4.0 | 3.7 | 4.0 | 5.1 | 6.8 | 6.9 | na | 10 |
| OR Quart 3 | 3.1 | 1.7 | 2.7 | 2.3 | 1.3 | 3.6 | 3.3 | >5.5 | 3.5 |
| p Value | 0.033 | 0.48 | 0.072 | 0.12 | 0.71 | 0.023 | 0.10 | <0.13 | 0.15 |
| 95% CI of | 1.1 | 0.39 | 0.92 | 0.80 | 0.29 | 1.2 | 0.78 | >0.62 | 0.65 |
| OR Quart 3 | 8.6 | 7.4 | 7.8 | 6.5 | 6.2 | 11 | 14 | na | 19 |
| OR Quart 4 | 1.1 | 1.3 | 1.2 | 1.4 | 2.9 | 1.7 | 1.0 | >1.0 | 2.2 |
| p Value | 0.83 | 0.71 | 0.77 | 0.58 | 0.13 | 0.39 | 1.0 | <1.0 | 0.37 |
| 95% CI of | 0.38 | 0.29 | 0.38 | 0.46 | 0.72 | 0.52 | 0.18 | >0.061 | 0.38 |
| OR Quart 4 | 3.4 | 6.2 | 3.7 | 4.0 | 11 | 5.3 | 5.4 | na | 13 |

Interleukin-2 receptor alpha chain

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 18.1 | 22.6 | 18.1 | 18.3 | 18.1 | 20.6 |
| Average | 38.7 | 84.1 | 38.7 | 115 | 38.7 | 90.3 |
| Stdev | 51.1 | 146 | 51.1 | 221 | 51.1 | 180 |
| p (t-test) | | 0.0079 | | 0.0018 | | 0.020 |
| Min | 0.0290 | 0.0290 | 0.0290 | 0.0290 | 0.0290 | 0.0515 |
| Max | 232 | 715 | 232 | 1040 | 232 | 714 |
| n (Samp) | 94 | 45 | 94 | 42 | 94 | 18 |
| n (Patient) | 65 | 45 | 65 | 42 | 65 | 18 |
| sCr only | | | | | | |
| Median | 18.3 | 47.9 | 18.3 | 42.6 | 18.3 | 22.6 |
| Average | 61.1 | 86.0 | 61.1 | 95.8 | 61.1 | 148 |
| Stdev | 121 | 135 | 121 | 174 | 121 | 282 |
| p (t-test) | | 0.46 | | 0.26 | | 0.049 |
| Min | 0.0290 | 0.0461 | 0.0290 | 0.0290 | 0.0290 | 0.0290 |
| Max | 1040 | 518 | 1040 | 715 | 1040 | 862 |
| n (Samp) | 225 | 14 | 225 | 18 | 225 | 9 |
| n (Patient) | 132 | 14 | 132 | 18 | 132 | 9 |
| UO only | | | | | | |
| Median | 29.5 | 13.6 | 29.5 | 39.6 | 29.5 | 24.1 |
| Average | 46.4 | 89.1 | 46.4 | 152 | 46.4 | 98.0 |
| Stdev | 53.6 | 159 | 53.6 | 261 | 53.6 | 192 |
| p (t-test) | | 0.018 | | 1.3E−4 | | 0.027 |
| Min | 0.0290 | 0.0290 | 0.0290 | 0.0290 | 0.0290 | 0.0515 |
| Max | 232 | 715 | 232 | 1040 | 232 | 714 |
| n (Samp) | 103 | 37 | 103 | 41 | 103 | 15 |
| n (Patient) | 64 | 37 | 64 | 41 | 64 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.56 | 0.60 | 0.51 | 0.56 | 0.58 | 0.57 | 0.56 | 0.53 | 0.54 |
| SE | 0.053 | 0.082 | 0.056 | 0.054 | 0.073 | 0.054 | 0.076 | 0.100 | 0.081 |
| p | 0.25 | 0.22 | 0.80 | 0.31 | 0.29 | 0.21 | 0.46 | 0.79 | 0.60 |
| nCohort 1 | 94 | 225 | 103 | 94 | 225 | 103 | 94 | 225 | 103 |
| nCohort 2 | 45 | 14 | 37 | 42 | 18 | 41 | 18 | 9 | 15 |
| Cutoff 1 | 2.97 | 8.15 | 2.33 | 0.0569 | 11.0 | 4.31 | 5.94 | 0.0569 | 14.2 |
| Sens 1 | 71% | 71% | 70% | 71% | 72% | 71% | 72% | 78% | 73% |
| Spec 1 | 32% | 40% | 27% | 24% | 43% | 30% | 37% | 21% | 40% |
| Cutoff 2 | 0.0515 | 2.19 | 0.0515 | 0.0515 | 0.0515 | 0.0515 | 0.0612 | 0.0515 | 5.94 |
| Sens 2 | 84% | 86% | 84% | 81% | 89% | 83% | 83% | 89% | 80% |
| Spec 2 | 17% | 30% | 17% | 17% | 16% | 17% | 29% | 16% | 31% |
| Cutoff 3 | 0.0359 | 0.0359 | 0.0290 | 0.0359 | 0.0290 | 0.0359 | 0.0515 | 0 | 0.0612 |
| Sens 3 | 91% | 100% | 95% | 90% | 94% | 95% | 94% | 100% | 93% |
| Spec 3 | 11% | 9% | 5% | 11% | 4% | 10% | 17% | 0% | 23% |
| Cutoff 4 | 41.0 | 45.8 | 56.7 | 41.0 | 45.8 | 56.7 | 41.0 | 45.8 | 56.7 |
| Sens 4 | 44% | 50% | 35% | 45% | 50% | 41% | 28% | 33% | 33% |
| Spec 4 | 70% | 70% | 71% | 70% | 70% | 71% | 70% | 70% | 71% |
| Cutoff 5 | 68.3 | 84.9 | 89.4 | 68.3 | 84.9 | 89.4 | 68.3 | 84.9 | 89.4 |
| Sens 5 | 36% | 36% | 27% | 31% | 22% | 34% | 28% | 33% | 27% |
| Spec 5 | 81% | 80% | 81% | 81% | 80% | 81% | 81% | 80% | 81% |
| Cutoff 6 | 117 | 153 | 132 | 117 | 153 | 132 | 117 | 153 | 132 |
| Sens 6 | 22% | 14% | 19% | 24% | 11% | 29% | 17% | 33% | 13% |
| Spec 6 | 90% | 90% | 90% | 90% | 90% | 90% | 90% | 90% | 90% |
| OR Quart 2 | 1.3 | 1.5 | 0.87 | 0.66 | 0.72 | 0.74 | 1.8 | 0.32 | 3.4 |
| p Value | 0.66 | 0.66 | 0.79 | 0.43 | 0.68 | 0.59 | 0.45 | 0.32 | 0.16 |
| 95% CI of | 0.45 | 0.24 | 0.30 | 0.23 | 0.16 | 0.25 | 0.39 | 0.032 | 0.62 |
| OR Quart 2 | 3.5 | 9.3 | 2.5 | 1.9 | 3.4 | 2.2 | 8.4 | 3.1 | 18 |
| OR Quart 3 | 0.60 | 1.5 | 0.62 | 0.39 | 0.98 | 0.87 | 1.8 | 0.65 | 1.6 |
| p Value | 0.37 | 0.66 | 0.41 | 0.10 | 0.98 | 0.79 | 0.45 | 0.65 | 0.64 |
| 95% CI of | 0.20 | 0.24 | 0.21 | 0.13 | 0.23 | 0.30 | 0.39 | 0.11 | 0.24 |
| OR Quart 3 | 1.8 | 9.3 | 1.9 | 1.2 | 4.1 | 2.5 | 8.4 | 4.1 | 10 |
| OR Quart 4 | 2.0 | 3.2 | 1.1 | 1.4 | 1.8 | 1.7 | 1.8 | 0.98 | 2.1 |
| p Value | 0.16 | 0.17 | 0.79 | 0.46 | 0.36 | 0.32 | 0.45 | 0.98 | 0.42 |
| 95% CI of | 0.75 | 0.61 | 0.41 | 0.55 | 0.50 | 0.61 | 0.39 | 0.19 | 0.35 |
| OR Quart 4 | 5.5 | 16 | 3.2 | 3.8 | 6.6 | 4.5 | 8.4 | 5.1 | 12 |

Protransforming growth factor alpha

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.51 | 1.45 | 1.51 | 2.44 | 1.51 | 4.16 |
| Average | 6.28 | 6.62 | 6.28 | 13.7 | 6.28 | 7.24 |
| Stdev | 30.0 | 24.2 | 30.0 | 43.6 | 30.0 | 9.10 |
| p (t-test) | | 0.95 | | 0.25 | | 0.89 |
| Min | 0.00228 | 0.00228 | 0.00228 | 0.00305 | 0.00228 | 0.00305 |
| Max | 287 | 162 | 287 | 245 | 287 | 30.3 |
| n (Samp) | 94 | 45 | 94 | 42 | 94 | 18 |
| n (Patient) | 65 | 45 | 65 | 42 | 65 | 18 |
| sCr only | | | | | | |
| Median | 1.47 | 2.13 | 1.47 | 3.26 | 1.47 | 0.885 |
| Average | 7.40 | 5.15 | 7.40 | 31.0 | 7.40 | 4.52 |
| Stdev | 27.1 | 8.71 | 27.1 | 67.7 | 27.1 | 9.72 |
| p (t-test) | | 0.76 | | 0.0027 | | 0.75 |
| Min | 0.00228 | 0.00228 | 0.00228 | 0.00305 | 0.00228 | 0.0105 |
| Max | 287 | 30.8 | 287 | 245 | 287 | 30.3 |
| n (Samp) | 225 | 14 | 225 | 18 | 225 | 9 |
| n (Patient) | 132 | 14 | 132 | 18 | 132 | 9 |
| UO only | | | | | | |
| Median | 1.22 | 1.26 | 1.22 | 2.41 | 1.22 | 4.80 |
| Average | 5.94 | 6.29 | 5.94 | 9.09 | 5.94 | 6.62 |
| Stdev | 28.7 | 26.3 | 28.7 | 25.6 | 28.7 | 7.52 |
| p (t-test) | | 0.95 | | 0.54 | | 0.93 |
| Min | 0.00228 | 0.00305 | 0.00228 | 0.00228 | 0.00228 | 0.00305 |
| Max | 287 | 162 | 287 | 153 | 287 | 28.3 |
| n (Samp) | 103 | 37 | 103 | 41 | 103 | 15 |
| n (Patient) | 64 | 37 | 64 | 41 | 64 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.51 | 0.50 | 0.51 | 0.58 | 0.63 | 0.59 | 0.71 | 0.49 | 0.73 |
| SE | 0.053 | 0.080 | 0.056 | 0.054 | 0.073 | 0.054 | 0.073 | 0.099 | 0.078 |
| p | 0.86 | 0.99 | 0.81 | 0.12 | 0.067 | 0.084 | 0.0047 | 0.90 | 0.0034 |
| nCohort 1 | 94 | 225 | 103 | 94 | 225 | 103 | 94 | 225 | 103 |
| nCohort 2 | 45 | 14 | 37 | 42 | 18 | 41 | 18 | 9 | 15 |
| Cutoff 1 | 0.597 | 0.490 | 0.597 | 0.666 | 0.875 | 0.875 | 1.35 | 0.674 | 3.84 |
| Sens 1 | 71% | 71% | 70% | 71% | 72% | 71% | 72% | 78% | 73% |
| Spec 1 | 37% | 29% | 41% | 38% | 38% | 46% | 48% | 34% | 81% |
| Cutoff 2 | 0.0907 | 0.00228 | 0.0907 | 0.490 | 0.637 | 0.141 | 0.875 | 0.666 | 1.35 |
| Sens 2 | 80% | 86% | 81% | 81% | 83% | 80% | 83% | 89% | 80% |
| Spec 2 | 21% | 4% | 22% | 32% | 34% | 27% | 41% | 34% | 52% |
| Cutoff 3 | 0.00305 | 0 | 0.00305 | 0.00305 | 0.0223 | 0.00305 | 0.340 | 0.00584 | 0.0907 |
| Sens 3 | 91% | 100% | 95% | 93% | 94% | 93% | 94% | 100% | 93% |
| Spec 3 | 15% | 0% | 17% | 15% | 20% | 17% | 29% | 17% | 22% |
| Cutoff 4 | 3.05 | 3.27 | 2.83 | 3.05 | 3.27 | 2.83 | 3.05 | 3.27 | 2.83 |
| Sens 4 | 24% | 29% | 19% | 38% | 50% | 41% | 67% | 22% | 73% |
| Spec 4 | 70% | 70% | 71% | 70% | 70% | 71% | 70% | 70% | 71% |
| Cutoff 5 | 3.96 | 4.80 | 3.84 | 3.96 | 4.80 | 3.84 | 3.96 | 4.80 | 3.84 |
| Sens 5 | 24% | 29% | 19% | 33% | 39% | 32% | 56% | 11% | 73% |
| Spec 5 | 81% | 80% | 81% | 81% | 80% | 81% | 81% | 80% | 81% |
| Cutoff 6 | 6.50 | 9.07 | 7.79 | 6.50 | 9.07 | 7.79 | 6.50 | 9.07 | 7.79 |
| Sens 6 | 13% | 14% | 8% | 24% | 33% | 22% | 28% | 11% | 20% |
| Spec 6 | 90% | 90% | 90% | 90% | 90% | 90% | 90% | 90% | 90% |
| OR Quart 2 | 1.4 | 0.47 | 1.4 | 1.6 | 2.6 | 1.3 | 5.9 | 2.1 | 0.96 |
| p Value | 0.50 | 0.40 | 0.59 | 0.42 | 0.27 | 0.59 | 0.12 | 0.56 | 0.97 |
| 95% CI of | 0.52 | 0.083 | 0.46 | 0.53 | 0.48 | 0.46 | 0.64 | 0.18 | 0.13 |
| OR Quart 2 | 3.9 | 2.7 | 4.0 | 4.5 | 14 | 3.9 | 54 | 23 | 7.3 |
| OR Quart 3 | 1.1 | 0.98 | 1.8 | 1.2 | 1.5 | 1.3 | 2.1 | 5.4 | 0.48 |
| p Value | 0.86 | 0.98 | 0.29 | 0.78 | 0.66 | 0.59 | 0.56 | 0.13 | 0.56 |
| 95% CI of | 0.39 | 0.23 | 0.61 | 0.39 | 0.24 | 0.46 | 0.18 | 0.61 | 0.041 |
| OR Quart 3 | 3.1 | 4.1 | 5.0 | 3.5 | 9.3 | 3.9 | 24 | 47 | 5.6 |
| OR Quart 4 | 1.1 | 0.98 | 0.84 | 2.3 | 4.4 | 2.0 | 15 | 1.0 | 6.8 |
| p Value | 0.86 | 0.98 | 0.77 | 0.12 | 0.069 | 0.20 | 0.013 | 0.99 | 0.021 |
| 95% CI of | 0.39 | 0.23 | 0.27 | 0.80 | 0.89 | 0.70 | 1.8 | 0.062 | 1.3 |
| OR Quart 4 | 3.1 | 4.1 | 2.6 | 6.5 | 22 | 5.6 | 130 | 17 | 34 |

CA 15-3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.248 | 0.246 | 0.248 | 0.380 | 0.248 | 0.210 |
| Average | 0.476 | 0.678 | 0.476 | 1.88 | 0.476 | 3.33 |
| Stdev | 0.801 | 1.63 | 0.801 | 6.00 | 0.801 | 12.1 |
| p (t-test) | | 0.28 | | 0.012 | | 0.010 |
| Min | 0.00165 | 0.00165 | 0.00165 | 0.0177 | 0.00165 | 0.0355 |
| Max | 6.54 | 11.0 | 6.54 | 36.7 | 6.54 | 60.0 |
| n (Samp) | 121 | 53 | 121 | 53 | 121 | 26 |
| n (Patient) | 87 | 53 | 87 | 53 | 87 | 26 |
| sCr only | | | | | | |
| Median | 0.276 | 0.254 | 0.276 | 0.528 | 0.276 | 0.360 |
| Average | 1.11 | 0.413 | 1.11 | 1.07 | 1.11 | 0.876 |
| Stdev | 4.63 | 0.570 | 4.63 | 2.34 | 4.63 | 1.70 |
| p (t-test) | | 0.55 | | 0.98 | | 0.87 |
| Min | 0.00165 | 0.0294 | 0.00165 | 0.0463 | 0.00165 | 0.0355 |
| Max | 60.0 | 2.27 | 60.0 | 9.48 | 60.0 | 5.66 |
| n (Samp) | 288 | 16 | 288 | 15 | 288 | 10 |
| n (Patient) | 161 | 16 | 161 | 15 | 161 | 10 |
| UO only | | | | | | |
| Median | 0.292 | 0.246 | 0.292 | 0.395 | 0.292 | 0.399 |
| Average | 0.541 | 0.734 | 0.541 | 1.88 | 0.541 | 3.76 |
| Stdev | 0.887 | 1.78 | 0.887 | 5.95 | 0.887 | 12.8 |
| p (t-test) | | 0.36 | | 0.015 | | 0.0055 |
| Min | 0.00165 | 0.00165 | 0.00165 | 0.0177 | 0.00165 | 0.0642 |
| Max | 6.54 | 11.0 | 6.54 | 36.7 | 6.54 | 60.0 |
| n (Samp) | 125 | 43 | 125 | 54 | 125 | 23 |
| n (Patient) | 80 | 43 | 80 | 54 | 80 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.51 | 0.43 | 0.49 | 0.62 | 0.62 | 0.60 | 0.52 | 0.49 | 0.54 |
| SE | 0.048 | 0.076 | 0.051 | 0.047 | 0.079 | 0.047 | 0.063 | 0.093 | 0.067 |
| p | 0.78 | 0.35 | 0.85 | 0.012 | 0.14 | 0.036 | 0.76 | 0.95 | 0.51 |
| nCohort 1 | 121 | 288 | 125 | 121 | 288 | 125 | 121 | 288 | 125 |
| nCohort 2 | 53 | 16 | 43 | 53 | 15 | 54 | 26 | 10 | 23 |
| Cutoff 1 | 0.171 | 0.0734 | 0.172 | 0.203 | 0.345 | 0.235 | 0.126 | 0.147 | 0.148 |
| Sens 1 | 72% | 75% | 72% | 72% | 73% | 70% | 73% | 70% | 74% |
| Spec 1 | 32% | 6% | 28% | 40% | 60% | 41% | 25% | 24% | 26% |
| Cutoff 2 | 0.126 | 0.0598 | 0.136 | 0.143 | 0.187 | 0.186 | 0.107 | 0.100 | 0.126 |
| Sens 2 | 81% | 81% | 81% | 83% | 80% | 81% | 81% | 80% | 83% |
| Spec 2 | 25% | 5% | 22% | 26% | 32% | 30% | 22% | 15% | 22% |
| Cutoff 3 | 0.0598 | 0.0294 | 0.0734 | 0.117 | 0.117 | 0.127 | 0.0762 | 0.0762 | 0.107 |
| Sens 3 | 91% | 94% | 91% | 91% | 93% | 91% | 92% | 90% | 91% |
| Spec 3 | 7% | 2% | 8% | 23% | 18% | 22% | 8% | 7% | 20% |
| Cutoff 4 | 0.373 | 0.447 | 0.411 | 0.373 | 0.447 | 0.411 | 0.373 | 0.447 | 0.411 |
| Sens 4 | 34% | 19% | 33% | 51% | 60% | 48% | 46% | 50% | 48% |
| Spec 4 | 70% | 70% | 70% | 70% | 70% | 70% | 70% | 70% | 70% |
| Cutoff 5 | 0.536 | 0.685 | 0.720 | 0.536 | 0.685 | 0.720 | 0.536 | 0.685 | 0.720 |
| Sens 5 | 25% | 12% | 16% | 34% | 27% | 26% | 27% | 30% | 22% |
| Spec 5 | 80% | 80% | 80% | 80% | 80% | 80% | 80% | 80% | 80% |
| Cutoff 6 | 1.08 | 1.41 | 1.14 | 1.08 | 1.41 | 1.14 | 1.08 | 1.41 | 1.14 |
| Sens 6 | 9% | 6% | 9% | 17% | 7% | 19% | 15% | 10% | 17% |
| Spec 6 | 90% | 90% | 90% | 90% | 90% | 90% | 90% | 90% | 90% |
| OR Quart 2 | 1.3 | 1.4 | 0.77 | 0.75 | 0.32 | 1.2 | 0.97 | 0.24 | 0.81 |
| p Value | 0.53 | 0.70 | 0.61 | 0.57 | 0.33 | 0.67 | 0.95 | 0.21 | 0.74 |
| 95% CI of | 0.54 | 0.29 | 0.28 | 0.27 | 0.033 | 0.47 | 0.30 | 0.027 | 0.22 |
| OR Quart 2 | 3.3 | 6.3 | 2.1 | 2.0 | 3.1 | 3.2 | 3.1 | 2.2 | 2.9 |
| OR Quart 3 | 0.89 | 1.4 | 1.3 | 1.4 | 1.3 | 1.7 | 0.24 | 0.24 | 0.63 |
| p Value | 0.81 | 0.70 | 0.63 | 0.48 | 0.71 | 0.27 | 0.086 | 0.21 | 0.50 |
| 95% CI of | 0.34 | 0.29 | 0.49 | 0.55 | 0.29 | 0.66 | 0.046 | 0.026 | 0.16 |
| OR Quart 3 | 2.3 | 6.3 | 3.3 | 3.6 | 6.2 | 4.3 | 1.2 | 2.2 | 2.4 |
| OR Quart 4 | 1.3 | 1.7 | 0.88 | 2.2 | 2.4 | 2.1 | 1.5 | 1.0 | 1.4 |
| p Value | 0.53 | 0.47 | 0.80 | 0.087 | 0.21 | 0.13 | 0.45 | 0.98 | 0.55 |
| 95% CI of | 0.54 | 0.39 | 0.33 | 0.89 | 0.61 | 0.82 | 0.51 | 0.24 | 0.44 |
| OR Quart 4 | 3.3 | 7.4 | 2.4 | 5.5 | 9.8 | 5.2 | 4.6 | 4.2 | 4.6 |

TABLE 6
Comparison of marker levels in EDTA samples collected from Cohort 1 (patients
that did not progress beyond RIFLE stage 0 or R) and in EDTA samples collected
from subjects at 0, 24 hours, and 48 hours prior to reaching stage I or F in Cohort 2.
C-C motif chemokine 18

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 172 | 215 | 172 | 248 | 172 | 220 |
| Average | 240 | 229 | 240 | 325 | 240 | 302 |
| Stdev | 220 | 100 | 220 | 300 | 220 | 325 |
| p (t-test) | | 0.85 | | 0.062 | | 0.24 |
| Min | 31.7 | 51.5 | 31.7 | 92.4 | 31.7 | 103 |
| Max | 2110 | 378 | 2110 | 1650 | 2110 | 1550 |
| n (Samp) | 282 | 16 | 282 | 28 | 282 | 20 |
| n (Patient) | 160 | 16 | 160 | 28 | 160 | 20 |
| sCr only | | | | | | |
| Median | nd | nd | nd | nd | 180 | 266 |
| Average | nd | nd | nd | nd | 252 | 280 |
| Stdev | nd | nd | nd | nd | 237 | 101 |
| p (t-test) | nd | nd | nd | nd | | 0.77 |
| Min | nd | nd | nd | nd | 31.7 | 154 |
| Max | nd | nd | nd | nd | 2110 | 422 |
| n (Samp) | nd | nd | nd | nd | 353 | 6 |
| n (Patient) | nd | nd | nd | nd | 193 | 6 |
| UO only | | | | | | |
| Median | 176 | 238 | 176 | 254 | 176 | 198 |
| Average | 251 | 231 | 251 | 328 | 251 | 310 |
| Stdev | 226 | 108 | 226 | 307 | 226 | 354 |
| p (t-test) | | 0.75 | | 0.11 | | 0.32 |
| Min | 37.8 | 51.5 | 37.8 | 92.4 | 37.8 | 103 |
| Max | 2110 | 378 | 2110 | 1650 | 2110 | 1550 |
| n (Samp) | 258 | 13 | 258 | 26 | 258 | 17 |
| n (Patient) | 140 | 13 | 140 | 26 | 140 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.57 | nd | 0.55 | 0.64 | nd | 0.63 | 0.59 | 0.67 | 0.55 |
| SE | 0.076 | nd | 0.084 | 0.059 | nd | 0.061 | 0.069 | 0.12 | 0.074 |
| p | 0.33 | nd | 0.56 | 0.020 | nd | 0.040 | 0.18 | 0.17 | 0.46 |
| nCohort 1 | 282 | nd | 258 | 282 | nd | 258 | 282 | 353 | 258 |
| nCohort 2 | 16 | nd | 13 | 28 | nd | 26 | 20 | 6 | 17 |
| Cutoff 1 | 161 | nd | 159 | 169 | nd | 189 | 152 | 207 | 120 |
| Sens 1 | 75% | nd | 77% | 71% | nd | 73% | 70% | 83% | 71% |
| Spec 1 | 45% | nd | 42% | 48% | nd | 55% | 41% | 59% | 30% |
| Cutoff 2 | 159 | nd | 144 | 132 | nd | 150 | 118 | 207 | 117 |
| Sens 2 | 81% | nd | 85% | 82% | nd | 81% | 80% | 83% | 82% |
| Spec 2 | 44% | nd | 38% | 36% | nd | 40% | 31% | 59% | 29% |
| Cutoff 3 | 87.9 | nd | 87.9 | 107 | nd | 107 | 105 | 152 | 103 |
| Sens 3 | 94% | nd | 92% | 93% | nd | 92% | 90% | 100% | 94% |
| Spec 3 | 19% | nd | 18% | 27% | nd | 26% | 26% | 39% | 24% |
| Cutoff 4 | 268 | nd | 278 | 268 | nd | 278 | 268 | 276 | 278 |
| Sens 4 | 38% | nd | 38% | 46% | nd | 46% | 40% | 50% | 35% |
| Spec 4 | 70% | nd | 70% | 70% | nd | 70% | 70% | 70% | 70% |
| Cutoff 5 | 345 | nd | 384 | 345 | nd | 384 | 345 | 347 | 384 |
| Sens 5 | 19% | nd | 0% | 32% | nd | 19% | 15% | 33% | 12% |
| Spec 5 | 80% | nd | 80% | 80% | nd | 80% | 80% | 80% | 80% |
| Cutoff 6 | 497 | nd | 537 | 497 | nd | 537 | 497 | 497 | 537 |
| Sens 6 | 0% | nd | 0% | 11% | nd | 12% | 10% | 0% | 12% |
| Spec 6 | 90% | nd | 90% | 90% | nd | 90% | 90% | 90% | 90% |
| OR Quart 2 | 2.0 | nd | 1.5 | 3.7 | nd | 1.4 | 1.3 | >1.0 | 1.3 |
| p Value | 0.42 | nd | 0.66 | 0.11 | nd | 0.70 | 0.71 | <1.0 | 0.71 |
| 95% CI of | 0.36 | nd | 0.24 | 0.74 | nd | 0.29 | 0.29 | >0.062 | 0.29 |
| OR Quart 2 | 11 | nd | 9.3 | 18 | nd | 6.3 | 6.2 | na | 6.2 |
| OR Quart 3 | 2.6 | nd | 2.0 | 4.3 | nd | 3.3 | 2.1 | >2.0 | 1.7 |
| p Value | 0.26 | nd | 0.42 | 0.069 | nd | 0.084 | 0.31 | <0.57 | 0.48 |
| 95% CI of | 0.49 | nd | 0.36 | 0.89 | nd | 0.85 | 0.50 | >0.18 | 0.39 |
| OR Quart 3 | 14 | nd | 11 | 21 | nd | 13 | 8.7 | na | 7.4 |
| OR Quart 4 | 2.6 | nd | 2.0 | 6.2 | nd | 3.7 | 2.4 | >3.1 | 1.7 |
| p Value | 0.27 | nd | 0.42 | 0.021 | nd | 0.054 | 0.21 | <0.34 | 0.48 |
| 95% CI of | 0.48 | nd | 0.36 | 1.3 | nd | 0.98 | 0.61 | >0.31 | 0.39 |
| OR Quart 4 | 14 | nd | 11 | 29 | nd | 14 | 9.8 | na | 7.4 |

C-C motif chemokine 24

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 259 | 392 | 259 | 198 | 259 | 323 |
| Average | 397 | 544 | 397 | 325 | 397 | 429 |
| Stdev | 402 | 578 | 402 | 311 | 402 | 404 |
| p (t-test) | | 0.17 | | 0.36 | | 0.75 |
| Min | 0.0260 | 40.3 | 0.0260 | 13.1 | 0.0260 | 12.4 |
| Max | 2920 | 2430 | 2920 | 1100 | 2920 | 1380 |
| n (Samp) | 216 | 16 | 216 | 28 | 216 | 17 |
| n (Patient) | 132 | 16 | 132 | 28 | 132 | 17 |
| UO only | | | | | | |
| Median | 270 | 238 | 270 | 173 | 270 | 323 |
| Average | 409 | 394 | 409 | 345 | 409 | 471 |
| Stdev | 412 | 311 | 412 | 478 | 412 | 461 |
| p (t-test) | | 0.90 | | 0.45 | | 0.58 |
| Min | 2.15 | 40.3 | 2.15 | 13.1 | 2.15 | 12.4 |
| Max | 2920 | 980 | 2920 | 2430 | 2920 | 1380 |
| n (Samp) | 198 | 13 | 198 | 29 | 198 | 15 |
| n (Patient) | 117 | 13 | 117 | 29 | 117 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.58 | nd | 0.51 | 0.43 | nd | 0.39 | 0.51 | nd | 0.50 |
| SE | 0.077 | nd | 0.083 | 0.059 | nd | 0.059 | 0.073 | nd | 0.078 |
| p | 0.29 | nd | 0.93 | 0.23 | nd | 0.064 | 0.89 | nd | 0.99 |
| nCohort 1 | 216 | nd | 198 | 216 | nd | 198 | 216 | nd | 198 |
| nCohort 2 | 16 | nd | 13 | 28 | nd | 29 | 17 | nd | 15 |
| Cutoff 1 | 201 | nd | 173 | 85.2 | nd | 80.9 | 181 | nd | 96.1 |
| Sens 1 | 75% | nd | 77% | 71% | nd | 72% | 71% | nd | 73% |
| Spec 1 | 38% | nd | 29% | 12% | nd | 11% | 33% | nd | 15% |
| Cutoff 2 | 171 | nd | 154 | 69.9 | nd | 67.2 | 85.2 | nd | 85.2 |
| Sens 2 | 81% | nd | 85% | 82% | nd | 83% | 82% | nd | 80% |
| Spec 2 | 31% | nd | 26% | 10% | nd | 9% | 12% | nd | 12% |
| Cutoff 3 | 67.2 | nd | 67.2 | 33.2 | nd | 25.7 | 25.7 | nd | 25.7 |
| Sens 3 | 94% | nd | 92% | 93% | nd | 93% | 94% | nd | 93% |
| Spec 3 | 10% | nd | 9% | 2% | nd | 2% | 2% | nd | 2% |
| Cutoff 4 | 438 | nd | 440 | 438 | nd | 440 | 438 | nd | 440 |
| Sens 4 | 50% | nd | 38% | 32% | nd | 28% | 41% | nd | 40% |
| Spec 4 | 70% | nd | 70% | 70% | nd | 70% | 70% | nd | 70% |
| Cutoff 5 | 580 | nd | 625 | 580 | nd | 625 | 580 | nd | 625 |
| Sens 5 | 44% | nd | 31% | 21% | nd | 14% | 24% | nd | 33% |
| Spec 5 | 80% | nd | 80% | 80% | nd | 80% | 80% | nd | 80% |
| Cutoff 6 | 881 | nd | 940 | 881 | nd | 940 | 881 | nd | 940 |
| Sens 6 | 12% | nd | 8% | 11% | nd | 3% | 18% | nd | 27% |
| Spec 6 | 90% | nd | 90% | 90% | nd | 90% | 90% | nd | 90% |
| OR Quart 2 | 2.6 | nd | 2.6 | 1.0 | nd | 1.2 | 0.38 | nd | 0.31 |
| p Value | 0.26 | nd | 0.27 | 1.0 | nd | 0.75 | 0.26 | nd | 0.17 |
| 95% CI of | 0.49 | nd | 0.48 | 0.30 | nd | 0.35 | 0.070 | nd | 0.060 |
| OR Quart 2 | 14 | nd | 14 | 3.3 | nd | 4.3 | 2.0 | nd | 1.6 |
| OR Quart 3 | 1.0 | nd | 0.48 | 0.82 | nd | 1.2 | 0.79 | nd | 0.31 |
| p Value | 1.0 | nd | 0.55 | 0.75 | nd | 0.75 | 0.73 | nd | 0.17 |
| 95% CI of | 0.14 | nd | 0.042 | 0.24 | nd | 0.35 | 0.20 | nd | 0.060 |
| OR Quart 3 | 7.3 | nd | 5.5 | 2.8 | nd | 4.3 | 3.1 | nd | 1.6 |
| OR Quart 4 | 3.8 | nd | 2.6 | 2.0 | nd | 2.8 | 1.2 | nd | 0.83 |
| p Value | 0.10 | nd | 0.27 | 0.20 | nd | 0.068 | 0.77 | nd | 0.78 |
| 95% CI of | 0.76 | nd | 0.48 | 0.69 | nd | 0.93 | 0.35 | nd | 0.24 |
| OR Quart 4 | 19 | nd | 14 | 5.9 | nd | 8.7 | 4.2 | nd | 2.9 |

C-C motif chemokine 8

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 12.7 | 14.8 | 12.7 | 13.7 | 12.7 | 17.5 |
| Average | 18.3 | 18.6 | 18.3 | 17.2 | 18.3 | 24.4 |
| Stdev | 26.5 | 10.7 | 26.5 | 18.2 | 26.5 | 17.8 |
| p (t-test) | | 0.97 | | 0.83 | | 0.36 |
| Min | 0.162 | 6.68 | 0.162 | 1.20 | 0.162 | 9.54 |
| Max | 180 | 41.0 | 180 | 101 | 180 | 72.1 |
| n (Samp) | 216 | 16 | 216 | 28 | 216 | 17 |
| n (Patient) | 132 | 16 | 132 | 28 | 132 | 17 |
| UO only | | | | | | |
| Median | 12.3 | 14.5 | 12.3 | 13.8 | 12.3 | 16.3 |
| Average | 19.1 | 16.6 | 19.1 | 16.6 | 19.1 | 23.8 |
| Stdev | 28.1 | 9.85 | 28.1 | 17.6 | 28.1 | 18.9 |
| p (t-test) | | 0.74 | | 0.63 | | 0.53 |
| Min | 0.162 | 6.68 | 0.162 | 1.20 | 0.162 | 9.54 |
| Max | 180 | 41.0 | 180 | 101 | 180 | 72.1 |
| n (Samp) | 198 | 13 | 198 | 29 | 198 | 15 |
| n (Patient) | 117 | 13 | 117 | 29 | 117 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.61 | nd | 0.58 | 0.53 | nd | 0.53 | 0.70 | nd | 0.68 |
| SE | 0.077 | nd | 0.085 | 0.059 | nd | 0.058 | 0.073 | nd | 0.078 |
| p | 0.14 | nd | 0.37 | 0.57 | nd | 0.56 | 0.0050 | nd | 0.021 |
| nCohort 1 | 216 | nd | 198 | 216 | nd | 198 | 216 | nd | 198 |
| nCohort 2 | 16 | nd | 13 | 28 | nd | 29 | 17 | nd | 15 |
| Cutoff 1 | 12.8 | nd | 9.69 | 10.7 | nd | 10.7 | 15.4 | nd | 13.7 |
| Sens 1 | 75% | nd | 85% | 71% | nd | 72% | 71% | nd | 73% |
| Spec 1 | 50% | nd | 35% | 39% | nd | 42% | 62% | nd | 56% |
| Cutoff 2 | 9.69 | nd | 9.69 | 7.48 | nd | 7.48 | 13.0 | nd | 13.0 |
| Sens 2 | 88% | nd | 85% | 82% | nd | 83% | 82% | nd | 80% |
| Spec 2 | 33% | nd | 35% | 24% | nd | 25% | 52% | nd | 54% |
| Cutoff 3 | 8.22 | nd | 8.22 | 4.81 | nd | 4.81 | 11.3 | nd | 11.3 |
| Sens 3 | 94% | nd | 92% | 93% | nd | 93% | 94% | nd | 93% |
| Spec 3 | 28% | nd | 29% | 18% | nd | 19% | 41% | nd | 44% |
| Cutoff 4 | 17.1 | nd | 17.0 | 17.1 | nd | 17.0 | 17.1 | nd | 17.0 |
| Sens 4 | 31% | nd | 23% | 25% | nd | 21% | 53% | nd | 40% |
| Spec 4 | 70% | nd | 70% | 70% | nd | 70% | 70% | nd | 70% |
| Cutoff 5 | 20.1 | nd | 20.3 | 20.1 | nd | 20.3 | 20.1 | nd | 20.3 |
| Sens 5 | 25% | nd | 15% | 21% | nd | 17% | 29% | nd | 27% |
| Spec 5 | 80% | nd | 80% | 80% | nd | 80% | 80% | nd | 80% |
| Cutoff 6 | 29.8 | nd | 33.4 | 29.8 | nd | 33.4 | 29.8 | nd | 33.4 |
| Sens 6 | 19% | nd | 8% | 11% | nd | 3% | 24% | nd | 20% |
| Spec 6 | 90% | nd | 91% | 90% | nd | 91% | 90% | nd | 91% |
| OR Quart 2 | 4.2 | nd | 3.1 | 0.82 | nd | 0.80 | >3.2 | nd | >2.1 |
| p Value | 0.20 | nd | 0.34 | 0.75 | nd | 0.73 | <0.32 | nd | <0.56 |
| 95% CI of | 0.46 | nd | 0.31 | 0.24 | nd | 0.23 | >0.32 | nd | >0.18 |
| OR Quart 2 | 39 | nd | 30 | 2.8 | nd | 2.8 | na | nd | na |
| OR Quart 3 | 7.8 | nd | 7.8 | 1.8 | nd | 2.2 | >9.3 | nd | >9.4 |
| p Value | 0.058 | nd | 0.060 | 0.29 | nd | 0.14 | <0.039 | nd | <0.038 |
| 95% CI of | 0.93 | nd | 0.92 | 0.61 | nd | 0.77 | >1.1 | nd | >1.1 |
| OR Quart 3 | 66 | nd | 65 | 5.3 | nd | 6.4 | na | nd | na |
| OR Quart 4 | 4.2 | nd | 2.0 | 1.2 | nd | 0.98 | >6.6 | nd | >5.4 |
| p Value | 0.20 | nd | 0.58 | 0.77 | nd | 0.97 | <0.086 | nd | <0.13 |
| 95% CI of | 0.46 | nd | 0.18 | 0.38 | nd | 0.30 | >0.77 | nd | >0.61 |
| OR Quart 4 | 39 | nd | 23 | 3.8 | nd | 3.2 | na | nd | na |

Cathepsin D

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 229000 | 205000 | 229000 | 234000 | 229000 | 249000 |
| Average | 257000 | 294000 | 257000 | 292000 | 257000 | 281000 |
| Stdev | 147000 | 295000 | 147000 | 170000 | 147000 | 135000 |
| p (t-test) | | 0.36 | | 0.24 | | 0.47 |
| Min | 34100 | 127000 | 34100 | 90500 | 34100 | 83000 |
| Max | 1020000 | 1350000 | 1020000 | 655000 | 1020000 | 561000 |
| n (Samp) | 281 | 16 | 281 | 28 | 281 | 20 |
| n (Patient) | 159 | 16 | 159 | 28 | 159 | 20 |
| sCr only | | | | | | |
| Median | nd | nd | nd | nd | 227000 | 309000 |
| Average | nd | nd | nd | nd | 260000 | 329000 |
| Stdev | nd | nd | nd | nd | 166000 | 197000 |
| p (t-test) | nd | nd | nd | nd | | 0.32 |
| Min | nd | nd | nd | nd | 34100 | 131000 |
| Max | nd | nd | nd | nd | 1870000 | 561000 |
| n (Samp) | nd | nd | nd | nd | 352 | 6 |
| n (Patient) | nd | nd | nd | nd | 192 | 6 |
| UO only | | | | | | |
| Median | 236000 | 218000 | 236000 | 267000 | 236000 | 262000 |
| Average | 266000 | 237000 | 266000 | 332000 | 266000 | 310000 |
| Stdev | 148000 | 92200 | 148000 | 261000 | 148000 | 146000 |
| p (t-test) | | 0.49 | | 0.046 | | 0.24 |
| Min | 54000 | 130000 | 54000 | 90500 | 54000 | 83000 |
| Max | 1020000 | 411000 | 1020000 | 1350000 | 1020000 | 655000 |
| n (Samp) | 257 | 13 | 257 | 26 | 257 | 17 |
| n (Patient) | 139 | 13 | 139 | 26 | 139 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.49 | nd | 0.48 | 0.55 | nd | 0.56 | 0.57 | 0.60 | 0.61 |
| SE | 0.075 | nd | 0.083 | 0.058 | nd | 0.061 | 0.069 | 0.12 | 0.075 |
| p | 0.92 | nd | 0.76 | 0.42 | nd | 0.36 | 0.29 | 0.42 | 0.15 |
| nCohort 1 | 281 | nd | 257 | 281 | nd | 257 | 281 | 352 | 257 |
| nCohort 2 | 16 | nd | 13 | 28 | nd | 26 | 20 | 6 | 17 |
| Cutoff 1 | 159000 | nd | 167000 | 148000 | nd | 154000 | 212000 | 157000 | 244000 |
| Sens 1 | 75% | nd | 77% | 71% | nd | 73% | 70% | 83% | 71% |
| Spec 1 | 30% | nd | 32% | 26% | nd | 25% | 44% | 29% | 54% |
| Cutoff 2 | 143000 | nd | 159000 | 141000 | nd | 142000 | 169000 | 157000 | 212000 |
| Sens 2 | 81% | nd | 85% | 82% | nd | 81% | 80% | 83% | 82% |
| Spec 2 | 24% | nd | 28% | 22% | nd | 20% | 34% | 29% | 42% |
| Cutoff 3 | 129000 | nd | 137000 | 126000 | nd | 126000 | 157000 | 130000 | 166000 |
| Sens 3 | 94% | nd | 92% | 93% | nd | 92% | 90% | 100% | 94% |
| Spec 3 | 18% | nd | 19% | 16% | nd | 13% | 30% | 17% | 31% |
| Cutoff 4 | 305000 | nd | 310000 | 305000 | nd | 310000 | 305000 | 306000 | 310000 |
| Sens 4 | 31% | nd | 23% | 43% | nd | 38% | 35% | 50% | 35% |
| Spec 4 | 70% | nd | 70% | 70% | nd | 70% | 70% | 70% | 70% |
| Cutoff 5 | 357000 | nd | 374000 | 357000 | nd | 374000 | 357000 | 352000 | 374000 |
| Sens 5 | 19% | nd | 8% | 32% | nd | 35% | 25% | 50% | 29% |
| Spec 5 | 80% | nd | 80% | 80% | nd | 80% | 80% | 80% | 80% |
| Cutoff 6 | 450000 | nd | 469000 | 450000 | nd | 469000 | 450000 | 450000 | 469000 |
| Sens 6 | 6% | nd | 0% | 21% | nd | 19% | 15% | 33% | 18% |
| Spec 6 | 90% | nd | 90% | 90% | nd | 90% | 90% | 90% | 90% |
| OR Quart 2 | 0.49 | nd | 0.67 | 0.60 | nd | 0.68 | 3.2 | 2.0 | 4.1 |
| p Value | 0.42 | nd | 0.66 | 0.39 | nd | 0.53 | 0.17 | 0.57 | 0.21 |
| 95% CI of | 0.088 | nd | 0.11 | 0.19 | nd | 0.21 | 0.62 | 0.18 | 0.45 |
| OR Quart 2 | 2.8 | nd | 4.1 | 1.9 | nd | 2.3 | 16 | 22 | 38 |
| OR Quart 3 | 1.6 | nd | 2.1 | 0.73 | nd | 0.68 | 3.2 | 0 | 7.7 |
| p Value | 0.50 | nd | 0.31 | 0.58 | nd | 0.53 | 0.17 | na | 0.060 |
| 95% CI of | 0.42 | nd | 0.50 | 0.24 | nd | 0.21 | 0.62 | na | 0.92 |
| OR Quart 3 | 5.8 | nd | 8.8 | 2.2 | nd | 2.3 | 16 | na | 64 |
| OR Quart 4 | 1.0 | nd | 0.67 | 1.1 | nd | 1.3 | 3.1 | 3.0 | 5.2 |
| p Value | 0.98 | nd | 0.66 | 0.82 | nd | 0.62 | 0.17 | 0.34 | 0.14 |
| 95% CI of | 0.24 | nd | 0.11 | 0.41 | nd | 0.46 | 0.61 | 0.31 | 0.60 |
| OR Quart 4 | 4.2 | nd | 4.1 | 3.1 | nd | 3.7 | 16 | 30 | 46 |

C—X—C motif chemokine 13

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 47.3 | 51.3 | 47.3 | 50.8 | 47.3 | 47.6 |
| Average | 124 | 177 | 124 | 105 | 124 | 180 |
| Stdev | 237 | 487 | 237 | 245 | 237 | 278 |
| p (t-test) | | 0.44 | | 0.69 | | 0.36 |
| Min | 9.90 | 15.3 | 9.90 | 12.9 | 9.90 | 12.5 |
| Max | 1790 | 2000 | 1790 | 1340 | 1790 | 918 |
| n (Samp) | 216 | 16 | 216 | 28 | 216 | 17 |
| n (Patient) | 132 | 16 | 132 | 28 | 132 | 17 |
| UO only | | | | | | |
| Median | 52.2 | 49.8 | 52.2 | 53.6 | 52.2 | 50.0 |
| Average | 123 | 54.6 | 123 | 103 | 123 | 159 |
| Stdev | 218 | 26.9 | 218 | 241 | 218 | 247 |
| p (t-test) | | 0.26 | | 0.64 | | 0.55 |
| Min | 9.90 | 15.3 | 9.90 | 12.9 | 9.90 | 12.5 |
| Max | 1790 | 109 | 1790 | 1340 | 1790 | 918 |
| n (Samp) | 198 | 13 | 198 | 29 | 198 | 15 |
| n (Patient) | 117 | 13 | 117 | 29 | 117 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.49 | nd | 0.44 | 0.50 | nd | 0.48 | 0.51 | nd | 0.51 |
| SE | 0.075 | nd | 0.085 | 0.058 | nd | 0.058 | 0.073 | nd | 0.078 |
| p | 0.94 | nd | 0.51 | 0.94 | nd | 0.69 | 0.89 | nd | 0.85 |
| nCohort 1 | 216 | nd | 198 | 216 | nd | 198 | 216 | nd | 198 |
| nCohort 2 | 16 | nd | 13 | 28 | nd | 29 | 17 | nd | 15 |
| Cutoff 1 | 35.2 | nd | 35.2 | 39.1 | nd | 39.1 | 34.2 | nd | 34.2 |
| Sens 1 | 75% | nd | 77% | 71% | nd | 72% | 71% | nd | 73% |
| Spec 1 | 37% | nd | 34% | 42% | nd | 40% | 36% | nd | 33% |
| Cutoff 2 | 34.2 | nd | 34.2 | 29.3 | nd | 29.3 | 27.8 | nd | 27.8 |
| Sens 2 | 81% | nd | 85% | 82% | nd | 83% | 82% | nd | 80% |
| Spec 2 | 36% | nd | 33% | 30% | nd | 27% | 25% | nd | 23% |
| Cutoff 3 | 25.2 | nd | 24.9 | 23.5 | nd | 23.5 | 15.4 | nd | 15.4 |
| Sens 3 | 94% | nd | 92% | 93% | nd | 93% | 94% | nd | 93% |
| Spec 3 | 22% | nd | 19% | 20% | nd | 18% | 6% | nd | 6% |
| Cutoff 4 | 102 | nd | 106 | 102 | nd | 106 | 102 | nd | 106 |
| Sens 4 | 12% | nd | 8% | 14% | nd | 10% | 29% | nd | 33% |
| Spec 4 | 70% | nd | 70% | 70% | nd | 70% | 70% | nd | 70% |
| Cutoff 5 | 140 | nd | 141 | 140 | nd | 141 | 140 | nd | 141 |
| Sens 5 | 6% | nd | 0% | 7% | nd | 3% | 29% | nd | 33% |
| Spec 5 | 80% | nd | 80% | 80% | nd | 80% | 80% | nd | 80% |
| Cutoff 6 | 251 | nd | 254 | 251 | nd | 254 | 251 | nd | 254 |
| Sens 6 | 6% | nd | 0% | 4% | nd | 3% | 24% | nd | 20% |
| Spec 6 | 90% | nd | 90% | 90% | nd | 90% | 90% | nd | 90% |
| OR Quart 2 | 9.1 | nd | >6.8 | 3.1 | nd | 4.8 | 1.7 | nd | 1.0 |
| p Value | 0.040 | nd | <0.082 | 0.063 | nd | 0.020 | 0.47 | nd | 1.0 |
| 95% CI of | 1.1 | nd | >0.79 | 0.94 | nd | 1.3 | 0.39 | nd | 0.24 |
| OR Quart 2 | 75 | nd | na | 10 | nd | 18 | 7.6 | nd | 4.2 |
| OR Quart 3 | 5.4 | nd | >5.5 | 2.5 | nd | 3.4 | 1.4 | nd | 0.48 |
| p Value | 0.13 | nd | <0.12 | 0.15 | nd | 0.080 | 0.70 | nd | 0.41 |
| 95% CI of | 0.61 | nd | >0.62 | 0.72 | nd | 0.86 | 0.29 | nd | 0.084 |
| OR Quart 3 | 48 | nd | na | 8.5 | nd | 13 | 6.4 | nd | 2.7 |
| OR Quart 4 | 2.0 | nd | >2.1 | 1.0 | nd | 1.8 | 1.7 | nd | 1.2 |
| p Value | 0.57 | nd | <0.54 | 1.0 | nd | 0.45 | 0.48 | nd | 0.75 |
| 95% CI of | 0.18 | nd | >0.19 | 0.24 | nd | 0.40 | 0.39 | nd | 0.32 |
| OR Quart 4 | 23 | nd | na | 4.2 | nd | 7.8 | 7.5 | nd | 4.9 |

Insulin-like growth factor-binding protein 3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3220 | 2570 | 3220 | 3020 | 3220 | 2890 |
| Average | 3530 | 3330 | 3530 | 3480 | 3530 | 3450 |
| Stdev | 1570 | 1940 | 1570 | 1530 | 1570 | 2040 |
| p (t-test) | | 0.63 | | 0.87 | | 0.83 |
| Min | 823 | 698 | 823 | 651 | 823 | 730 |
| Max | 8600 | 6560 | 8600 | 7360 | 8600 | 7520 |
| n (Samp) | 282 | 16 | 282 | 28 | 282 | 20 |
| n (Patient) | 160 | 16 | 160 | 28 | 160 | 20 |
| sCr only | | | | | | |
| Median | nd | nd | nd | nd | 3090 | 3030 |
| Average | nd | nd | nd | nd | 3480 | 3870 |
| Stdev | nd | nd | nd | nd | 1600 | 1920 |
| p (t-test) | nd | nd | nd | nd | | 0.55 |
| Min | nd | nd | nd | nd | 651 | 2160 |
| Max | nd | nd | nd | nd | 8600 | 6560 |
| n (Samp) | nd | nd | nd | nd | 353 | 6 |
| n (Patient) | nd | nd | nd | nd | 193 | 6 |
| UO only | | | | | | |
| Median | 3060 | 2280 | 3060 | 3020 | 3060 | 3020 |
| Average | 3410 | 2650 | 3410 | 3540 | 3410 | 3240 |
| Stdev | 1460 | 1430 | 1460 | 1610 | 1460 | 1950 |
| p (t-test) | | 0.071 | | 0.66 | | 0.66 |
| Min | 823 | 698 | 823 | 651 | 823 | 730 |
| Max | 8260 | 6250 | 8260 | 7360 | 8260 | 7520 |
| n (Samp) | 258 | 13 | 258 | 26 | 258 | 17 |
| n (Patient) | 140 | 13 | 140 | 26 | 140 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.44 | nd | 0.34 | 0.50 | nd | 0.53 | 0.46 | 0.56 | 0.44 |
| SE | 0.076 | nd | 0.084 | 0.057 | nd | 0.060 | 0.068 | 0.12 | 0.074 |
| p | 0.43 | nd | 0.052 | 0.99 | nd | 0.67 | 0.57 | 0.62 | 0.45 |
| nCohort 1 | 282 | nd | 258 | 282 | nd | 258 | 282 | 353 | 258 |
| nCohort 2 | 16 | nd | 13 | 28 | nd | 26 | 20 | 6 | 17 |
| Cutoff 1 | 2130 | nd | 1950 | 2410 | nd | 2410 | 2370 | 2430 | 2040 |
| Sens 1 | 75% | nd | 77% | 71% | nd | 73% | 70% | 83% | 71% |
| Spec 1 | 21% | nd | 15% | 28% | nd | 29% | 27% | 31% | 19% |
| Cutoff 2 | 1950 | nd | 1610 | 2220 | nd | 2260 | 1880 | 2430 | 1590 |
| Sens 2 | 81% | nd | 85% | 82% | nd | 81% | 80% | 83% | 82% |
| Spec 2 | 15% | nd | 7% | 23% | nd | 24% | 12% | 31% | 7% |
| Cutoff 3 | 1210 | nd | 1210 | 2130 | nd | 2130 | 1490 | 2150 | 730 |
| Sens 3 | 94% | nd | 92% | 93% | nd | 92% | 90% | 100% | 94% |
| Spec 3 | 2% | nd | 3% | 21% | nd | 22% | 6% | 22% | 0% |
| Cutoff 4 | 4250 | nd | 4130 | 4250 | nd | 4130 | 4250 | 4230 | 4130 |
| Sens 4 | 25% | nd | 8% | 36% | nd | 38% | 30% | 33% | 29% |
| Spec 4 | 70% | nd | 70% | 70% | nd | 70% | 70% | 70% | 70% |
| Cutoff 5 | 4870 | nd | 4680 | 4870 | nd | 4680 | 4870 | 4800 | 4680 |
| Sens 5 | 25% | nd | 8% | 14% | nd | 27% | 20% | 33% | 18% |
| Spec 5 | 80% | nd | 80% | 80% | nd | 80% | 80% | 80% | 80% |
| Cutoff 6 | 5740 | nd | 5510 | 5740 | nd | 5510 | 5740 | 5740 | 5510 |
| Sens 6 | 25% | nd | 8% | 7% | nd | 8% | 20% | 33% | 12% |
| Spec 6 | 90% | nd | 90% | 90% | nd | 90% | 90% | 90% | 90% |
| OR Quart 2 | 0.75 | nd | 3.1 | 0.86 | nd | 1.2 | 0.59 | 2.0 | 1.7 |
| p Value | 0.71 | nd | 0.33 | 0.79 | nd | 0.77 | 0.48 | 0.57 | 0.47 |
| 95% CI of | 0.16 | nd | 0.31 | 0.27 | nd | 0.38 | 0.14 | 0.18 | 0.39 |
| OR Quart 2 | 3.5 | nd | 30 | 2.7 | nd | 3.7 | 2.6 | 22 | 7.5 |
| OR Quart 3 | 0.49 | nd | 3.1 | 1.2 | nd | 0.65 | 1.2 | 0.99 | 1.0 |
| p Value | 0.41 | nd | 0.33 | 0.79 | nd | 0.51 | 0.75 | 0.99 | 1.0 |
| 95% CI of | 0.086 | nd | 0.31 | 0.40 | nd | 0.17 | 0.36 | 0.061 | 0.19 |
| OR Quart 3 | 2.7 | nd | 30 | 3.4 | nd | 2.4 | 4.2 | 16 | 5.1 |
| OR Quart 4 | 1.9 | nd | 6.6 | 1.0 | nd | 1.6 | 1.2 | 2.0 | 2.1 |
| p Value | 0.34 | nd | 0.085 | 0.98 | nd | 0.42 | 0.74 | 0.57 | 0.30 |
| 95% CI of | 0.52 | nd | 0.77 | 0.34 | nd | 0.53 | 0.36 | 0.18 | 0.51 |
| OR Quart 4 | 6.6 | nd | 56 | 3.0 | nd | 4.7 | 4.2 | 22 | 8.9 |

Immunoglogulin G1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 8800000 | 7170000 | 8800000 | 1.04E7 | 8800000 | 9000000 |
| Average | 1.04E7 | 7280000 | 1.04E7 | 1.29E7 | 1.04E7 | 9890000 |
| Stdev | 5920000 | 1700000 | 5920000 | 7430000 | 5920000 | 4770000 |
| p (t-test) | | 0.12 | | 0.076 | | 0.82 |
| Min | 2060000 | 4390000 | 2060000 | 4620000 | 2060000 | 4390000 |
| Max | 4.18E7 | 9590000 | 4.18E7 | 2.98E7 | 4.18E7 | 2.05E7 |
| n (Samp) | 205 | 9 | 205 | 20 | 205 | 8 |
| n (Patient) | 127 | 9 | 127 | 20 | 127 | 8 |
| UO only | | | | | | |
| Median | 8690000 | 7170000 | 8690000 | 1.01E7 | 8690000 | 8470000 |
| Average | 1.01E7 | 7280000 | 1.01E7 | 1.21E7 | 1.01E7 | 9950000 |
| Stdev | 5930000 | 1700000 | 5930000 | 6630000 | 5930000 | 5150000 |
| p (t-test) | | 0.15 | | 0.19 | | 0.94 |
| Min | 2060000 | 4390000 | 2060000 | 4620000 | 2060000 | 4390000 |
| Max | 4.18E7 | 9590000 | 4.18E7 | 2.63E7 | 4.18E7 | 2.05E7 |
| n (Samp) | 191 | 9 | 191 | 18 | 191 | 7 |
| n (Patient) | 113 | 9 | 113 | 18 | 113 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.34 | nd | 0.36 | 0.61 | nd | 0.60 | 0.51 | nd | 0.52 |
| SE | 0.10 | nd | 0.10 | 0.070 | nd | 0.073 | 0.10 | nd | 0.11 |
| p | 0.11 | nd | 0.17 | 0.11 | nd | 0.15 | 0.94 | nd | 0.89 |
| nCohort 1 | 205 | nd | 191 | 205 | nd | 191 | 205 | nd | 191 |
| nCohort 2 | 9 | nd | 9 | 20 | nd | 18 | 8 | nd | 7 |
| Cutoff 1 | 6940000 | nd | 6940000 | 7810000 | nd | 7810000 | 8010000 | nd | 8010000 |
| Sens 1 | 78% | nd | 78% | 75% | nd | 72% | 75% | nd | 71% |
| Spec 1 | 28% | nd | 31% | 40% | nd | 43% | 44% | nd | 47% |
| Cutoff 2 | 5340000 | nd | 5340000 | 7160000 | nd | 7140000 | 6940000 | nd | 6940000 |
| Sens 2 | 89% | nd | 89% | 80% | nd | 83% | 88% | nd | 86% |
| Spec 2 | 13% | nd | 14% | 34% | nd | 34% | 28% | nd | 31% |
| Cutoff 3 | 4360000 | nd | 4360000 | 6940000 | nd | 6940000 | 4360000 | nd | 4360000 |
| Sens 3 | 100% | nd | 100% | 95% | nd | 94% | 100% | nd | 100% |
| Spec 3 | 5% | nd | 6% | 28% | nd | 31% | 5% | nd | 6% |
| Cutoff 4 | 1.13E7 | nd | 1.11E7 | 1.13E7 | nd | 1.11E7 | 1.13E7 | nd | 1.11E7 |
| Sens 4 | 0% | nd | 0% | 40% | nd | 39% | 25% | nd | 29% |
| Spec 4 | 71% | nd | 70% | 71% | nd | 70% | 71% | nd | 70% |
| Cutoff 5 | 1.38E7 | nd | 1.33E7 | 1.38E7 | nd | 1.33E7 | 1.38E7 | nd | 1.33E7 |
| Sens 5 | 0% | nd | 0% | 25% | nd | 22% | 12% | nd | 14% |
| Spec 5 | 80% | nd | 80% | 80% | nd | 80% | 80% | nd | 80% |
| Cutoff 6 | 1.71E7 | nd | 1.63E7 | 1.71E7 | nd | 1.63E7 | 1.71E7 | nd | 1.63E7 |
| Sens 6 | 0% | nd | 0% | 20% | nd | 22% | 12% | nd | 14% |
| Spec 6 | 90% | nd | 91% | 90% | nd | 91% | 90% | nd | 91% |
| OR Quart 2 | >2.1 | nd | >2.1 | 6.6 | nd | 6.7 | 3.1 | nd | 3.1 |
| p Value | <0.55 | nd | <0.55 | 0.086 | nd | 0.085 | 0.33 | nd | 0.34 |
| 95% CI of | >0.19 | nd | >0.18 | 0.77 | nd | 0.77 | 0.31 | nd | 0.31 |
| OR Quart 2 | na | nd | na | 57 | nd | 57 | 31 | nd | 31 |
| OR Quart 3 | >5.5 | nd | >5.6 | 9.2 | nd | 6.7 | 3.1 | nd | 2.0 |
| p Value | <0.13 | nd | <0.12 | 0.040 | nd | 0.085 | 0.33 | nd | 0.57 |
| 95% CI of | >0.62 | nd | >0.63 | 1.1 | nd | 0.77 | 0.31 | nd | 0.18 |
| OR Quart 3 | na | nd | na | 76 | nd | 57 | 31 | nd | 23 |
| OR Quart 4 | >2.1 | nd | >2.1 | 5.3 | nd | 5.3 | 0.98 | nd | 0.98 |
| p Value | <0.55 | nd | <0.55 | 0.13 | nd | 0.13 | 0.99 | nd | 0.99 |
| 95% CI of | >0.19 | nd | >0.18 | 0.60 | nd | 0.60 | 0.060 | nd | 0.060 |
| OR Quart 4 | na | nd | na | 47 | nd | 47 | 16 | nd | 16 |

Immunoglogulin G2

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.08E7 | 1.02E7 | 1.08E7 | 1.02E7 | 1.08E7 | 1.05E7 |
| Average | 1.37E7 | 8150000 | 1.37E7 | 1.05E7 | 1.37E7 | 1.01E7 |
| Stdev | 9640000 | 6300000 | 9640000 | 5640000 | 9640000 | 5710000 |
| p (t-test) | | 0.090 | | 0.15 | | 0.30 |
| Min | 190000 | 1750000 | 190000 | 1750000 | 190000 | 1750000 |
| Max | 5.80E7 | 1.70E7 | 5.80E7 | 2.70E7 | 5.80E7 | 1.70E7 |
| n (Samp) | 205 | 9 | 205 | 20 | 205 | 8 |
| n (Patient) | 127 | 9 | 127 | 20 | 127 | 8 |
| UO only | | | | | | |
| Median | 1.03E7 | 1.02E7 | 1.03E7 | 1.02E7 | 1.03E7 | 1.02E7 |
| Average | 1.33E7 | 8150000 | 1.33E7 | 1.05E7 | 1.33E7 | 1.00E7 |
| Stdev | 9390000 | 6300000 | 9390000 | 5580000 | 9390000 | 6160000 |
| p (t-test) | | 0.11 | | 0.22 | | 0.37 |
| Min | 190000 | 1750000 | 190000 | 1750000 | 190000 | 1750000 |
| Max | 5.80E7 | 1.70E7 | 5.80E7 | 2.70E7 | 5.80E7 | 1.70E7 |
| n (Samp) | 191 | 9 | 191 | 18 | 191 | 7 |
| n (Patient) | 113 | 9 | 113 | 18 | 113 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.34 | nd | 0.35 | 0.39 | nd | 0.40 | 0.41 | nd | 0.41 |
| SE | 0.10 | nd | 0.10 | 0.070 | nd | 0.073 | 0.11 | nd | 0.12 |
| p | 0.10 | nd | 0.13 | 0.11 | nd | 0.16 | 0.38 | nd | 0.42 |
| nCohort 1 | 205 | nd | 191 | 205 | nd | 191 | 205 | nd | 191 |
| nCohort 2 | 9 | nd | 9 | 20 | nd | 18 | 8 | nd | 7 |
| Cutoff 1 | 1930000 | nd | 1930000 | 8850000 | nd | 8850000 | 7850000 | nd | 7850000 |
| Sens 1 | 78% | nd | 78% | 70% | nd | 72% | 75% | nd | 71% |
| Spec 1 | 9% | nd | 9% | 27% | nd | 28% | 25% | nd | 27% |
| Cutoff 2 | 563000 | nd | 219000 | 6700000 | nd | 6700000 | 1930000 | nd | 1930000 |
| Sens 2 | 100% | nd | 100% | 80% | nd | 83% | 88% | nd | 86% |
| Spec 2 | 1% | nd | 1% | 20% | nd | 21% | 9% | nd | 9% |
| Cutoff 3 | 563000 | nd | 219000 | 4550000 | nd | 219000 | 563000 | nd | 219000 |
| Sens 3 | 100% | nd | 100% | 90% | nd | 100% | 100% | nd | 100% |
| Spec 3 | 1% | nd | 1% | 18% | nd | 1% | 1% | nd | 1% |
| Cutoff 4 | 1.64E7 | nd | 1.64E7 | 1.64E7 | nd | 1.64E7 | 1.64E7 | nd | 1.64E7 |
| Sens 4 | 22% | nd | 22% | 10% | nd | 6% | 12% | nd | 14% |
| Spec 4 | 71% | nd | 72% | 71% | nd | 72% | 71% | nd | 72% |
| Cutoff 5 | 1.85E7 | nd | 1.84E7 | 1.85E7 | nd | 1.84E7 | 1.85E7 | nd | 1.84E7 |
| Sens 5 | 0% | nd | 0% | 5% | nd | 6% | 0% | nd | 0% |
| Spec 5 | 80% | nd | 80% | 80% | nd | 80% | 80% | nd | 80% |
| Cutoff 6 | 2.55E7 | nd | 2.52E7 | 2.55E7 | nd | 2.52E7 | 2.55E7 | nd | 2.52E7 |
| Sens 6 | 0% | nd | 0% | 5% | nd | 6% | 0% | nd | 0% |
| Spec 6 | 90% | nd | 90% | 90% | nd | 90% | 90% | nd | 90% |
| OR Quart 2 | 1.0 | nd | 0 | 6.7 | nd | 6.8 | 3.2 | nd | 2.1 |
| p Value | 0.99 | nd | na | 0.083 | nd | 0.082 | 0.32 | nd | 0.55 |
| 95% CI of | 0.062 | nd | na | 0.78 | nd | 0.79 | 0.32 | nd | 0.18 |
| OR Quart 2 | 17 | nd | na | 58 | nd | 58 | 32 | nd | 24 |
| OR Quart 3 | 3.1 | nd | 1.5 | 9.3 | nd | 8.1 | 2.1 | nd | 2.0 |
| p Value | 0.33 | nd | 0.65 | 0.038 | nd | 0.055 | 0.56 | nd | 0.57 |
| 95% CI of | 0.31 | nd | 0.24 | 1.1 | nd | 0.96 | 0.18 | nd | 0.18 |
| OR Quart 3 | 31 | nd | 9.6 | 77 | nd | 68 | 24 | nd | 23 |
| OR Quart 4 | 4.3 | nd | 2.1 | 5.5 | nd | 4.3 | 2.1 | nd | 2.1 |
| p Value | 0.20 | nd | 0.41 | 0.13 | nd | 0.20 | 0.56 | nd | 0.55 |
| 95% CI of | 0.47 | nd | 0.36 | 0.62 | nd | 0.47 | 0.18 | nd | 0.18 |
| OR Quart 4 | 40 | nd | 12 | 49 | nd | 40 | 24 | nd | 24 |

Interleukin-11

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 67.2 | 37.8 | 67.2 | 89.1 | 67.2 | 51.1 |
| Average | 162 | 185 | 162 | 98.0 | 162 | 109 |
| Stdev | 635 | 477 | 635 | 75.8 | 635 | 142 |
| p (t-test) | | 0.89 | | 0.60 | | 0.73 |
| Min | 0.359 | 0.442 | 0.359 | 0.480 | 0.359 | 0.480 |
| Max | 6920 | 1940 | 6920 | 392 | 6920 | 569 |
| n (Samp) | 217 | 16 | 217 | 28 | 217 | 17 |
| n (Patient) | 133 | 16 | 133 | 28 | 133 | 17 |
| UO only | | | | | | |
| Median | 62.2 | 33.9 | 62.2 | 90.7 | 62.2 | 55.3 |
| Average | 156 | 47.3 | 156 | 86.9 | 156 | 88.3 |
| Stdev | 660 | 52.1 | 660 | 50.4 | 660 | 80.0 |
| p (t-test) | | 0.55 | | 0.57 | | 0.69 |
| Min | 0.359 | 0.442 | 0.359 | 0.480 | 0.359 | 0.480 |
| Max | 6920 | 176 | 6920 | 198 | 6920 | 233 |
| n (Samp) | 199 | 13 | 199 | 29 | 199 | 15 |
| n (Patient) | 118 | 13 | 118 | 29 | 118 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.43 | nd | 0.37 | 0.57 | nd | 0.57 | 0.51 | nd | 0.53 |
| SE | 0.077 | nd | 0.085 | 0.059 | nd | 0.059 | 0.073 | nd | 0.079 |
| p | 0.36 | nd | 0.14 | 0.24 | nd | 0.21 | 0.91 | nd | 0.70 |
| nCohort 1 | 217 | nd | 199 | 217 | nd | 199 | 217 | nd | 199 |
| nCohort 2 | 16 | nd | 13 | 28 | nd | 29 | 17 | nd | 15 |
| Cutoff 1 | 7.90 | nd | 6.00 | 55.3 | nd | 55.3 | 33.9 | nd | 33.9 |
| Sens 1 | 75% | nd | 77% | 71% | nd | 72% | 71% | nd | 80% |
| Spec 1 | 21% | nd | 18% | 46% | nd | 47% | 35% | nd | 36% |
| Cutoff 2 | 6.00 | nd | 0.608 | 47.0 | nd | 31.8 | 20.2 | nd | 33.9 |
| Sens 2 | 81% | nd | 85% | 82% | nd | 83% | 82% | nd | 80% |
| Spec 2 | 17% | nd | 12% | 44% | nd | 32% | 27% | nd | 36% |
| Cutoff 3 | 0.442 | nd | 0.442 | 15.1 | nd | 7.90 | 0.608 | nd | 6.07 |
| Sens 3 | 94% | nd | 92% | 93% | nd | 93% | 94% | nd | 93% |
| Spec 3 | 8% | nd | 8% | 23% | nd | 22% | 12% | nd | 21% |
| Cutoff 4 | 118 | nd | 113 | 118 | nd | 113 | 118 | nd | 113 |
| Sens 4 | 19% | nd | 15% | 25% | nd | 21% | 29% | nd | 27% |
| Spec 4 | 70% | nd | 70% | 70% | nd | 70% | 70% | nd | 70% |
| Cutoff 5 | 163 | nd | 158 | 163 | nd | 158 | 163 | nd | 158 |
| Sens 5 | 19% | nd | 8% | 14% | nd | 10% | 29% | nd | 27% |
| Spec 5 | 80% | nd | 82% | 80% | nd | 82% | 80% | nd | 82% |
| Cutoff 6 | 246 | nd | 209 | 246 | nd | 209 | 246 | nd | 209 |
| Sens 6 | 12% | nd | 0% | 4% | nd | 0% | 6% | nd | 13% |
| Spec 6 | 90% | nd | 90% | 90% | nd | 90% | 90% | nd | 90% |
| OR Quart 2 | 0.67 | nd | 2.0 | 2.1 | nd | 1.3 | 2.5 | nd | 3.2 |
| p Value | 0.66 | nd | 0.57 | 0.31 | nd | 0.73 | 0.21 | nd | 0.17 |
| 95% CI of | 0.11 | nd | 0.18 | 0.50 | nd | 0.32 | 0.61 | nd | 0.61 |
| OR Quart 2 | 4.1 | nd | 23 | 8.9 | nd | 5.0 | 10 | nd | 17 |
| OR Quart 3 | 1.8 | nd | 6.6 | 5.2 | nd | 4.7 | 0.65 | nd | 1.5 |
| p Value | 0.45 | nd | 0.085 | 0.013 | nd | 0.0095 | 0.65 | nd | 0.65 |
| 95% CI of | 0.40 | nd | 0.77 | 1.4 | nd | 1.5 | 0.11 | nd | 0.25 |
| OR Quart 3 | 7.7 | nd | 57 | 19 | nd | 15 | 4.1 | nd | 9.5 |
| OR Quart 4 | 2.2 | nd | 4.2 | 2.1 | nd | 1.3 | 1.7 | nd | 2.0 |
| p Value | 0.30 | nd | 0.20 | 0.32 | nd | 0.73 | 0.48 | nd | 0.42 |
| 95% CI of | 0.51 | nd | 0.46 | 0.49 | nd | 0.32 | 0.39 | nd | 0.36 |
| OR Quart 4 | 9.1 | nd | 39 | 8.7 | nd | 5.0 | 7.5 | nd | 12 |

Interleukin-2 receptor alpha chain

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 18.3 | 18.7 | 18.3 | 25.1 | 18.3 | 22.6 |
| Average | 59.5 | 84.0 | 59.5 | 65.2 | 59.5 | 121 |
| Stdev | 126 | 139 | 126 | 140 | 126 | 224 |
| p (t-test) | | 0.46 | | 0.82 | | 0.072 |
| Min | 0.0290 | 0.0290 | 0.0290 | 0.0290 | 0.0290 | 0.0461 |
| Max | 1040 | 518 | 1040 | 715 | 1040 | 862 |
| n (Samp) | 217 | 16 | 217 | 28 | 217 | 17 |
| n (Patient) | 133 | 16 | 133 | 28 | 133 | 17 |
| UO only | | | | | | |
| Median | 24.1 | 23.9 | 24.1 | 28.8 | 24.1 | 30.0 |
| Average | 67.8 | 95.2 | 67.8 | 84.5 | 67.8 | 135 |
| Stdev | 136 | 152 | 136 | 162 | 136 | 236 |
| p (t-test) | | 0.49 | | 0.55 | | 0.084 |
| Min | 0.0290 | 0.0290 | 0.0290 | 0.0290 | 0.0290 | 0.0461 |
| Max | 1040 | 518 | 1040 | 715 | 1040 | 862 |
| n (Samp) | 199 | 13 | 199 | 29 | 199 | 15 |
| n (Patient) | 118 | 13 | 118 | 29 | 118 | 15 |

| | 0 hr prior to AKI stage | | | 24 hr prior to AKI stage | | | hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.57 | nd | 0.55 | 0.50 | nd | 0.52 | 0.56 | nd | 0.56 |
| SE | 0.077 | nd | 0.085 | 0.058 | nd | 0.058 | 0.075 | nd | 0.079 |
| p | 0.38 | nd | 0.52 | 0.99 | nd | 0.78 | 0.43 | nd | 0.47 |
| nCohort 1 | 217 | nd | 199 | 217 | nd | 199 | 217 | nd | 199 |
| nCohort 2 | 16 | nd | 13 | 28 | nd | 29 | 17 | nd | 15 |
| Cutoff 1 | 10.6 | nd | 11.0 | 0.0515 | nd | 6.51 | 3.62 | nd | 3.62 |
| Sens 1 | 75% | nd | 77% | 75% | nd | 72% | 71% | nd | 73% |
| Spec 1 | 42% | nd | 38% | 17% | nd | 34% | 32% | nd | 29% |
| Cutoff 2 | 6.86 | nd | 10.6 | 0.0429 | nd | 0.0359 | 0.0569 | nd | 0.0612 |
| Sens 2 | 81% | nd | 85% | 86% | nd | 86% | 88% | nd | 80% |
| Spec 2 | 39% | nd | 38% | 9% | nd | 9% | 23% | nd | 24% |
| Cutoff 3 | 1.56 | nd | 1.56 | 0.0290 | nd | 0.0290 | 0.0515 | nd | 0.0569 |
| Sens 3 | 94% | nd | 92% | 96% | nd | 97% | 94% | nd | 93% |
| Spec 3 | 30% | nd | 26% | 5% | nd | 6% | 17% | nd | 20% |
| Cutoff 4 | 48.3 | nd | 54.6 | 48.3 | nd | 54.6 | 48.3 | nd | 54.6 |
| Sens 4 | 31% | nd | 31% | 32% | nd | 38% | 35% | nd | 40% |
| Spec 4 | 70% | nd | 70% | 70% | nd | 70% | 70% | nd | 70% |
| Cutoff 5 | 88.9 | nd | 102 | 88.9 | nd | 102 | 88.9 | nd | 102 |
| Sens 5 | 31% | nd | 31% | 11% | nd | 14% | 29% | nd | 27% |
| Spec 5 | 80% | nd | 80% | 80% | nd | 80% | 80% | nd | 80% |
| Cutoff 6 | 148 | nd | 153 | 148 | nd | 153 | 148 | nd | 153 |
| Sens 6 | 19% | nd | 23% | 11% | nd | 14% | 24% | nd | 27% |
| Spec 6 | 90% | nd | 90% | 90% | nd | 90% | 90% | nd | 90% |
| OR Quart 2 | 7.8 | nd | 6.6 | 0.20 | nd | 0.59 | 0.98 | nd | 0.72 |
| p Value | 0.058 | nd | 0.085 | 0.043 | nd | 0.38 | 0.98 | nd | 0.68 |
| 95% CI of | 0.93 | nd | 0.77 | 0.040 | nd | 0.18 | 0.23 | nd | 0.15 |
| OR Quart 2 | 66 | nd | 57 | 0.95 | nd | 1.9 | 4.1 | nd | 3.4 |
| OR Quart 3 | 3.1 | nd | 2.0 | 0.87 | nd | 1.1 | 1.0 | nd | 0.74 |
| p Value | 0.33 | nd | 0.57 | 0.79 | nd | 0.79 | 1.0 | nd | 0.70 |
| 95% CI of | 0.31 | nd | 0.18 | 0.31 | nd | 0.41 | 0.24 | nd | 0.16 |
| OR Quart 3 | 31 | nd | 23 | 2.4 | nd | 3.2 | 4.2 | nd | 3.5 |
| OR Quart 4 | 5.3 | nd | 4.2 | 0.98 | nd | 0.86 | 1.2 | nd | 1.2 |
| p Value | 0.13 | nd | 0.20 | 0.97 | nd | 0.78 | 0.75 | nd | 0.75 |
| 95% CI of | 0.60 | nd | 0.46 | 0.36 | nd | 0.29 | 0.32 | nd | 0.32 |
| OR Quart 4 | 47 | nd | 39 | 2.7 | nd | 2.5 | 4.9 | nd | 4.9 |

Neutrophil collagenase

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1090 | 1320 | 1090 | 1800 | 1090 | 1160 |
| Average | 2990 | 1400 | 2990 | 5630 | 2990 | 4630 |
| Stdev | 6890 | 1170 | 6890 | 9430 | 6890 | 7310 |
| p (t-test) | | 0.36 | | 0.064 | | 0.31 |
| Min | 57.2 | 21.8 | 57.2 | 1.14 | 57.2 | 131 |
| Max | 55000 | 3920 | 55000 | 38700 | 55000 | 28000 |
| n (Samp) | 281 | 16 | 281 | 28 | 281 | 20 |
| n (Patient) | 160 | 16 | 160 | 28 | 160 | 20 |
| sCr only | | | | | | |
| Median | nd | nd | nd | nd | 1130 | 885 |
| Average | nd | nd | nd | nd | 3250 | 9310 |
| Stdev | nd | nd | nd | nd | 6960 | 15400 |
| p (t-test) | nd | nd | nd | nd | | 0.040 |
| Min | nd | nd | nd | nd | 57.2 | 264 |
| Max | nd | nd | nd | nd | 55000 | 38700 |
| n (Samp) | nd | nd | nd | nd | 352 | 6 |
| n (Patient) | nd | nd | nd | nd | 193 | 6 |
| UO only | | | | | | |
| Median | 1100 | 1420 | 1100 | 2360 | 1100 | 1160 |
| Average | 3390 | 1590 | 3390 | 5940 | 3390 | 4490 |
| Stdev | 7790 | 1190 | 7790 | 9700 | 7790 | 7460 |
| p (t-test) | | 0.41 | | 0.12 | | 0.57 |
| Min | 40.0 | 196 | 40.0 | 21.8 | 40.0 | 1.14 |
| Max | 55000 | 3920 | 55000 | 38700 | 55000 | 28000 |
| n (Samp) | 257 | 13 | 257 | 26 | 257 | 17 |
| n (Patient) | 140 | 13 | 140 | 26 | 140 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.47 | nd | 0.52 | 0.61 | nd | 0.62 | 0.55 | 0.51 | 0.54 |
| SE | 0.075 | nd | 0.083 | 0.059 | nd | 0.061 | 0.068 | 0.12 | 0.074 |
| p | 0.72 | nd | 0.83 | 0.065 | nd | 0.045 | 0.44 | 0.96 | 0.57 |
| nCohort 1 | 281 | nd | 257 | 281 | nd | 257 | 281 | 352 | 257 |
| nCohort 2 | 16 | nd | 13 | 28 | nd | 26 | 20 | 6 | 17 |
| Cutoff 1 | 520 | nd | 669 | 799 | nd | 906 | 792 | 618 | 792 |
| Sens 1 | 75% | nd | 77% | 71% | nd | 73% | 70% | 83% | 71% |
| Spec 1 | 21% | nd | 30% | 38% | nd | 42% | 37% | 26% | 37% |
| Cutoff 2 | 318 | nd | 520 | 554 | nd | 743 | 724 | 618 | 724 |
| Sens 2 | 81% | nd | 85% | 82% | nd | 81% | 80% | 83% | 82% |
| Spec 2 | 8% | nd | 21% | 23% | nd | 35% | 35% | 26% | 34% |
| Cutoff 3 | 193 | nd | 200 | 414 | nd | 414 | 451 | 261 | 131 |
| Sens 3 | 94% | nd | 92% | 93% | nd | 92% | 90% | 100% | 94% |
| Spec 3 | 2% | nd | 3% | 15% | nd | 14% | 17% | 5% | 2% |
| Cutoff 4 | 1700 | nd | 1830 | 1700 | nd | 1830 | 1700 | 1880 | 1830 |
| Sens 4 | 31% | nd | 31% | 50% | nd | 54% | 35% | 33% | 35% |
| Spec 4 | 70% | nd | 70% | 70% | nd | 70% | 70% | 70% | 70% |
| Cutoff 5 | 2830 | nd | 3110 | 2830 | nd | 3110 | 2830 | 3180 | 3110 |
| Sens 5 | 12% | nd | 15% | 39% | nd | 38% | 25% | 33% | 24% |
| Spec 5 | 80% | nd | 80% | 80% | nd | 80% | 80% | 80% | 80% |
| Cutoff 6 | 4950 | nd | 5930 | 4950 | nd | 5930 | 4950 | 7160 | 5930 |
| Sens 6 | 0% | nd | 0% | 29% | nd | 31% | 25% | 33% | 24% |
| Spec 6 | 90% | nd | 90% | 90% | nd | 90% | 90% | 90% | 90% |
| OR Quart 2 | 1.3 | nd | 0.65 | 0.82 | nd | 0.78 | 2.1 | 3.0 | 1.3 |
| p Value | 0.72 | nd | 0.64 | 0.75 | nd | 0.71 | 0.31 | 0.34 | 0.71 |
| 95% CI of | 0.33 | nd | 0.10 | 0.24 | nd | 0.20 | 0.50 | 0.31 | 0.29 |
| OR Quart 2 | 5.0 | nd | 4.0 | 2.8 | nd | 3.0 | 8.7 | 30 | 6.2 |
| OR Quart 3 | 0.49 | nd | 1.7 | 0.65 | nd | 0.78 | 1.4 | 0 | 1.4 |
| p Value | 0.42 | nd | 0.47 | 0.52 | nd | 0.71 | 0.70 | na | 0.70 |
| 95% CI of | 0.088 | nd | 0.39 | 0.18 | nd | 0.20 | 0.29 | na | 0.29 |
| OR Quart 3 | 2.8 | nd | 7.5 | 2.4 | nd | 3.0 | 6.3 | na | 6.3 |
| OR Quart 4 | 1.3 | nd | 0.98 | 2.4 | nd | 2.9 | 2.4 | 2.0 | 2.1 |
| p Value | 0.72 | nd | 0.99 | 0.099 | nd | 0.055 | 0.21 | 0.57 | 0.32 |
| 95% CI of | 0.33 | nd | 0.19 | 0.85 | nd | 0.98 | 0.61 | 0.18 | 0.49 |
| OR Quart 4 | 5.0 | nd | 5.1 | 6.6 | nd | 8.7 | 9.8 | 22 | 8.6 |

Protransforming growth factor alpha

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.45 | 1.67 | 1.45 | 2.22 | 1.45 | 2.57 |
| Average | 5.56 | 16.8 | 5.56 | 13.3 | 5.56 | 21.2 |
| Stdev | 26.1 | 35.1 | 26.1 | 32.4 | 26.1 | 41.5 |
| p (t-test) | | 0.11 | | 0.15 | | 0.025 |
| Min | 0.00228 | 0.00228 | 0.00228 | 0.00228 | 0.00228 | 0.00228 |
| Max | 287 | 137 | 287 | 162 | 287 | 153 |
| n (Samp) | 217 | 16 | 217 | 28 | 217 | 17 |
| n (Patient) | 133 | 16 | 133 | 28 | 133 | 17 |
| UO only | | | | | | |
| Median | 1.26 | 1.22 | 1.26 | 1.87 | 1.26 | 2.57 |
| Average | 5.63 | 17.9 | 5.63 | 12.3 | 5.63 | 21.8 |
| Stdev | 27.2 | 38.5 | 27.2 | 31.7 | 27.2 | 44.0 |
| p (t-test) | | 0.13 | | 0.23 | | 0.036 |
| Min | 0.00228 | 0.00228 | 0.00228 | 0.00228 | 0.00228 | 0.00228 |
| Max | 287 | 137 | 287 | 162 | 287 | 153 |
| n (Samp) | 199 | 13 | 199 | 29 | 199 | 15 |
| n (Patient) | 118 | 13 | 118 | 29 | 118 | 15 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.60 | nd | 0.58 | 0.61 | nd | 0.62 | 0.66 | nd | 0.64 |
| SE | 0.077 | nd | 0.085 | 0.060 | nd | 0.059 | 0.074 | nd | 0.080 |
| p | 0.21 | nd | 0.32 | 0.075 | nd | 0.033 | 0.036 | nd | 0.084 |
| nCohort 1 | 217 | nd | 199 | 217 | nd | 199 | 217 | nd | 199 |
| nCohort 2 | 16 | nd | 13 | 28 | nd | 29 | 17 | nd | 15 |
| Cutoff 1 | 0.597 | nd | 0.597 | 0.885 | nd | 0.885 | 1.70 | nd | 0.666 |
| Sens 1 | 75% | nd | 77% | 71% | nd | 72% | 71% | nd | 73% |
| Spec 1 | 35% | nd | 38% | 41% | nd | 44% | 54% | nd | 39% |
| Cutoff 2 | 0.540 | nd | 0.537 | 0.704 | nd | 0.704 | 0.674 | nd | 0.190 |
| Sens 2 | 81% | nd | 85% | 82% | nd | 83% | 82% | nd | 80% |
| Spec 2 | 35% | nd | 36% | 37% | nd | 40% | 37% | nd | 28% |
| Cutoff 3 | 0.0223 | nd | 0.0223 | 0.190 | nd | 0.309 | 0.00228 | nd | 0.00228 |
| Sens 3 | 94% | nd | 92% | 93% | nd | 93% | 94% | nd | 93% |
| Spec 3 | 19% | nd | 21% | 25% | nd | 31% | 5% | nd | 5% |
| Cutoff 4 | 3.05 | nd | 2.70 | 3.05 | nd | 2.70 | 3.05 | nd | 2.70 |
| Sens 4 | 44% | nd | 38% | 39% | nd | 38% | 47% | nd | 47% |
| Spec 4 | 71% | nd | 70% | 71% | nd | 70% | 71% | nd | 70% |
| Cutoff 5 | 4.18 | nd | 4.00 | 4.18 | nd | 4.00 | 4.18 | nd | 4.00 |
| Sens 5 | 38% | nd | 31% | 32% | nd | 31% | 47% | nd | 47% |
| Spec 5 | 80% | nd | 80% | 80% | nd | 80% | 80% | nd | 80% |
| Cutoff 6 | 6.50 | nd | 6.43 | 6.50 | nd | 6.43 | 6.50 | nd | 6.43 |
| Sens 6 | 31% | nd | 31% | 25% | nd | 24% | 41% | nd | 40% |
| Spec 6 | 90% | nd | 90% | 90% | nd | 90% | 90% | nd | 90% |
| OR Quart 2 | 3.2 | nd | 2.7 | 3.8 | nd | 5.9 | 0.64 | nd | 1.5 |
| p Value | 0.16 | nd | 0.26 | 0.052 | nd | 0.027 | 0.64 | nd | 0.66 |
| 95% CI of | 0.62 | nd | 0.49 | 0.99 | nd | 1.2 | 0.10 | nd | 0.24 |
| OR Quart 2 | 17 | nd | 14 | 15 | nd | 28 | 4.0 | nd | 9.4 |
| OR Quart 3 | 1.0 | nd | 1.0 | 1.7 | nd | 3.8 | 1.4 | nd | 1.5 |
| p Value | 1.0 | nd | 1.0 | 0.47 | nd | 0.10 | 0.70 | nd | 0.65 |
| 95% CI of | 0.14 | nd | 0.14 | 0.39 | nd | 0.76 | 0.29 | nd | 0.25 |
| OR Quart 3 | 7.3 | nd | 7.4 | 7.6 | nd | 19 | 6.4 | nd | 9.5 |
| OR Quart 4 | 3.2 | nd | 2.1 | 3.7 | nd | 5.9 | 2.9 | nd | 3.8 |
| p Value | 0.17 | nd | 0.41 | 0.055 | nd | 0.027 | 0.13 | nd | 0.11 |
| 95% CI of | 0.61 | nd | 0.36 | 0.97 | nd | 1.2 | 0.72 | nd | 0.75 |
| OR Quart 4 | 16 | nd | 12 | 14 | nd | 28 | 11 | nd | 19 |

CA 15-3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.253 | 0.375 | 0.253 | 0.552 | 0.253 | 0.502 |
| Average | 0.895 | 1.25 | 0.895 | 1.93 | 0.895 | 1.79 |
| Stdev | 4.27 | 3.04 | 4.27 | 4.80 | 4.27 | 4.30 |
| p (t-test) | | 0.75 | | 0.23 | | 0.37 |
| Min | 0.00165 | 0.00154 | 0.00165 | 0.0463 | 0.00165 | 0.0355 |
| Max | 60.0 | 12.5 | 60.0 | 24.0 | 60.0 | 17.8 |
| n (Samp) | 282 | 16 | 282 | 28 | 282 | 20 |
| n (Patient) | 160 | 16 | 160 | 28 | 160 | 20 |
| sCr only | | | | | | |
| Median | nd | nd | nd | nd | 0.292 | 0.334 |
| Average | nd | nd | nd | nd | 1.05 | 0.350 |
| Stdev | nd | nd | nd | nd | 4.23 | 0.248 |
| p (t-test) | nd | nd | nd | nd | | 0.69 |
| Min | nd | nd | nd | nd | 0.00154 | 0.0355 |
| Max | nd | nd | nd | nd | 60.0 | 0.682 |
| n (Samp) | nd | nd | nd | nd | 353 | 6 |
| n (Patient) | nd | nd | nd | nd | 193 | 6 |
| UO only | | | | | | |
| Median | 0.284 | 0.366 | 0.284 | 0.588 | 0.284 | 0.530 |
| Average | 0.965 | 1.40 | 0.965 | 2.08 | 0.965 | 2.07 |
| Stdev | 4.46 | 3.37 | 4.46 | 4.96 | 4.46 | 4.63 |
| p (t-test) | | 0.73 | | 0.23 | | 0.32 |
| Min | 0.00165 | 0.00154 | 0.00165 | 0.109 | 0.00165 | 0.141 |
| Max | 60.0 | 12.5 | 60.0 | 24.0 | 60.0 | 17.8 |
| n (Samp) | 258 | 13 | 258 | 26 | 258 | 17 |
| n (Patient) | 140 | 13 | 140 | 26 | 140 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.59 | nd | 0.58 | 0.66 | nd | 0.66 | 0.64 | 0.48 | 0.67 |
| SE | 0.077 | nd | 0.085 | 0.058 | nd | 0.061 | 0.069 | 0.12 | 0.074 |
| p | 0.25 | nd | 0.35 | 0.0064 | nd | 0.0078 | 0.035 | 0.87 | 0.019 |
| nCohort 1 | 282 | nd | 258 | 282 | nd | 258 | 282 | 353 | 258 |
| nCohort 2 | 16 | nd | 13 | 28 | nd | 26 | 20 | 6 | 17 |
| Cutoff 1 | 0.266 | nd | 0.261 | 0.249 | nd | 0.249 | 0.392 | 0.187 | 0.409 |
| Sens 1 | 75% | nd | 77% | 71% | nd | 73% | 70% | 83% | 71% |
| Spec 1 | 51% | nd | 47% | 50% | nd | 46% | 69% | 31% | 67% |
| Cutoff 2 | 0.191 | nd | 0.191 | 0.187 | nd | 0.191 | 0.176 | 0.187 | 0.176 |
| Sens 2 | 81% | nd | 85% | 82% | nd | 81% | 80% | 83% | 82% |
| Spec 2 | 34% | nd | 30% | 33% | nd | 30% | 32% | 31% | 28% |
| Cutoff 3 | 0.0177 | nd | 0.0177 | 0.133 | nd | 0.136 | 0.147 | 0.0338 | 0.147 |
| Sens 3 | 94% | nd | 92% | 93% | nd | 92% | 90% | 100% | 94% |
| Spec 3 | 2% | nd | 2% | 23% | nd | 21% | 25% | 3% | 22% |
| Cutoff 4 | 0.409 | nd | 0.456 | 0.409 | nd | 0.456 | 0.409 | 0.476 | 0.456 |
| Sens 4 | 38% | nd | 38% | 57% | nd | 58% | 65% | 50% | 59% |
| Spec 4 | 70% | nd | 70% | 70% | nd | 70% | 70% | 70% | 70% |
| Cutoff 5 | 0.614 | nd | 0.708 | 0.614 | nd | 0.708 | 0.614 | 0.701 | 0.708 |
| Sens 5 | 31% | nd | 23% | 46% | nd | 38% | 30% | 0% | 29% |
| Spec 5 | 80% | nd | 80% | 80% | nd | 80% | 80% | 80% | 80% |
| Cutoff 6 | 1.14 | nd | 1.20 | 1.14 | nd | 1.20 | 1.14 | 1.40 | 1.20 |
| Sens 6 | 19% | nd | 15% | 25% | nd | 31% | 15% | 0% | 18% |
| Spec 6 | 90% | nd | 90% | 90% | nd | 90% | 90% | 90% | 90% |
| OR Quart 2 | 0.32 | nd | 1.5 | 1.2 | nd | 1.7 | 0.99 | 2.0 | 0.99 |
| p Value | 0.33 | nd | 0.66 | 0.75 | nd | 0.47 | 0.99 | 0.57 | 0.99 |
| 95% CI of | 0.033 | nd | 0.24 | 0.32 | nd | 0.39 | 0.19 | 0.18 | 0.13 |
| OR Quart 2 | 3.1 | nd | 9.3 | 4.8 | nd | 7.5 | 5.0 | 23 | 7.2 |
| OR Quart 3 | 2.1 | nd | 1.5 | 1.3 | nd | 1.7 | 1.4 | 2.0 | 2.6 |
| p Value | 0.31 | nd | 0.66 | 0.73 | nd | 0.47 | 0.70 | 0.57 | 0.27 |
| 95% CI of | 0.50 | nd | 0.24 | 0.33 | nd | 0.39 | 0.29 | 0.18 | 0.48 |
| OR Quart 3 | 8.7 | nd | 9.3 | 4.9 | nd | 7.5 | 6.3 | 23 | 14 |
| OR Quart 4 | 2.1 | nd | 2.6 | 4.0 | nd | 5.1 | 3.6 | 1.0 | 4.3 |
| p Value | 0.32 | nd | 0.27 | 0.019 | nd | 0.015 | 0.058 | 0.99 | 0.071 |
| 95% CI of | 0.49 | nd | 0.48 | 1.3 | nd | 1.4 | 0.96 | 0.062 | 0.88 |
| OR Quart 4 | 8.6 | nd | 14 | 13 | nd | 19 | 14 | 16 | 21 |

TABLE 7
Comparison of marker levels in EDTA samples collected within 12
hours of reaching stage R from Cohort 1 (patients that reached, but did not progress
beyond, RIFLE stage R) and from Cohort 2 (patients that reached RIFLE stage I or F).
C-C motif chemokine 8

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 13.7 | 13.8 | nd | nd | 13.8 | 13.8 |
| Average | 19.7 | 14.2 | nd | nd | 13.8 | 13.4 |
| Stdev | 31.0 | 7.93 | nd | nd | 10.1 | 8.09 |
| p (t-test) | | 0.45 | nd | nd | | 0.88 |
| Min | 0.162 | 1.20 | nd | nd | 0.170 | 1.20 |
| Max | 166 | 34.9 | nd | nd | 50.3 | 34.9 |
| n (Samp) | 41 | 19 | nd | nd | 31 | 15 |
| n (Patient) | 41 | 19 | nd | nd | 31 | 15 |

| | At Enrollment | | |
|---|---|---|---|
| | sCr or UO | sCr only | UO only |
| AUC | 0.52 | nd | 0.50 |
| SE | 0.081 | nd | 0.092 |
| p | 0.77 | nd | 0.97 |
| nCohort 1 | 41 | nd | 31 |
| nCohort 2 | 19 | nd | 15 |
| Cutoff 1 | 10.7 | nd | 10.6 |
| Sens 1 | 74% | nd | 73% |
| Spec 1 | 41% | nd | 42% |
| Cutoff 2 | 7.48 | nd | 8.22 |
| Sens 2 | 84% | nd | 80% |
| Spec 2 | 22% | nd | 32% |
| Cutoff 3 | 1.16 | nd | 1.16 |
| Sens 3 | 100% | nd | 100% |
| Spec 3 | 7% | nd | 6% |
| Cutoff 4 | 16.9 | nd | 16.9 |
| Sens 4 | 26% | nd | 13% |
| Spec 4 | 71% | nd | 71% |
| Cutoff 5 | 18.9 | nd | 18.9 |
| Sens 5 | 21% | nd | 13% |
| Spec 5 | 80% | nd | 81% |
| Cutoff 6 | 26.8 | nd | 25.2 |
| Sens 6 | 5% | nd | 7% |
| Spec 6 | 90% | nd | 90% |
| OR Quart 2 | 1.4 | nd | 6.0 |
| p Value | 0.69 | nd | 0.068 |
| 95% CI of | 0.29 | nd | 0.87 |
| OR Quart 2 | 6.6 | nd | 41 |
| OR Quart 3 | 1.4 | nd | 2.5 |
| p Value | 0.69 | nd | 0.35 |
| 95% CI of | 0.29 | nd | 0.36 |
| OR Quart 3 | 6.6 | nd | 17 |
| OR Quart 4 | 1.4 | nd | 1.9 |
| p Value | 0.69 | nd | 0.54 |
| 95% CI of | 0.29 | nd | 0.25 |
| OR Quart 4 | 6.6 | nd | 14 |

Immunoglogulin G1

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 8820000 | 8470000 | nd | nd | 8840000 | 8470000 |
| Average | 9870000 | 1.04E7 | nd | nd | 9700000 | 8840000 |
| Stdev | 4230000 | 6820000 | nd | nd | 3800000 | 2660000 |
| p (t-test) | | 0.74 | nd | nd | | 0.58 |
| Min | 2060000 | 4620000 | nd | nd | 2060000 | 4620000 |
| Max | 2.33E7 | 2.72E7 | nd | nd | 1.75E7 | 1.20E7 |
| n (Samp) | 41 | 9 | nd | nd | 30 | 7 |
| n (Patient) | 41 | 9 | nd | nd | 30 | 7 |

| | At Enrollment | | |
|---|---|---|---|
| | sCr or UO | sCr only | UO only |
| AUC | 0.46 | nd | 0.45 |
| SE | 0.11 | nd | 0.12 |
| p | 0.73 | nd | 0.66 |
| nCohort 1 | 41 | nd | 30 |
| nCohort 2 | 9 | nd | 7 |
| Cutoff 1 | 7140000 | nd | 7600000 |
| Sens 1 | 78% | nd | 71% |
| Spec 1 | 29% | nd | 33% |
| Cutoff 2 | 4620000 | nd | 6710000 |
| Sens 2 | 89% | nd | 86% |
| Spec 2 | 2% | nd | 27% |
| Cutoff 3 | 2060000 | nd | 2060000 |
| Sens 3 | 100% | nd | 100% |
| Spec 3 | 2% | nd | 3% |
| Cutoff 4 | 1.17E7 | nd | 1.15E7 |
| Sens 4 | 33% | nd | 29% |
| Spec 4 | 71% | nd | 70% |
| Cutoff 5 | 1.34E7 | nd | 1.34E7 |
| Sens 5 | 11% | nd | 0% |
| Spec 5 | 80% | nd | 80% |
| Cutoff 6 | 1.59E7 | nd | 1.39E7 |
| Sens 6 | 11% | nd | 0% |
| Spec 6 | 90% | nd | 90% |
| OR Quart 2 | 4.0 | nd | 0.50 |
| p Value | 0.26 | nd | 0.60 |
| 95% CI of | 0.35 | nd | 0.037 |
| OR Quart 2 | 45 | nd | 6.7 |
| OR Quart 3 | 3.6 | nd | 2.0 |
| p Value | 0.30 | nd | 0.51 |
| 95% CI of | 0.32 | nd | 0.25 |
| OR Quart 3 | 40 | nd | 16 |
| OR Quart 4 | 2.4 | nd | 0.50 |
| p Value | 0.50 | nd | 0.60 |
| 95% CI of | 0.19 | nd | 0.037 |
| OR Quart 4 | 31 | nd | 6.7 |

Interleukin-11

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 91.3 | 87.4 | nd | nd | 91.9 | 76.6 |
| Average | 112 | 86.0 | nd | nd | 98.1 | 77.8 |
| Stdev | 130 | 49.3 | nd | nd | 86.1 | 52.9 |
| p (t-test) | | 0.40 | nd | nd | | 0.41 |
| Min | 0.359 | 0.480 | nd | nd | 0.359 | 0.480 |
| Max | 741 | 191 | nd | nd | 371 | 191 |
| n (Samp) | 41 | 19 | nd | nd | 31 | 15 |
| n (Patient) | 41 | 19 | nd | nd | 31 | 15 |

| | At Enrollment | | |
|---|---|---|---|
| | sCr or UO | sCr only | UO only |
| AUC | 0.47 | nd | 0.45 |
| SE | 0.081 | nd | 0.092 |
| p | 0.76 | nd | 0.61 |
| nCohort 1 | 41 | nd | 31 |
| nCohort 2 | 19 | nd | 15 |
| Cutoff 1 | 55.3 | nd | 47.0 |
| Sens 1 | 79% | nd | 73% |
| Spec 1 | 34% | nd | 35% |
| Cutoff 2 | 47.3 | nd | 10.7 |
| Sens 2 | 84% | nd | 80% |
| Spec 2 | 34% | nd | 26% |
| Cutoff 3 | 0.736 | nd | 0.736 |
| Sens 3 | 95% | nd | 93% |
| Spec 3 | 20% | nd | 26% |
| Cutoff 4 | 139 | nd | 146 |
| Sens 4 | 11% | nd | 7% |
| Spec 4 | 71% | nd | 71% |
| Cutoff 5 | 163 | nd | 163 |
| Sens 5 | 11% | nd | 7% |
| Spec 5 | 80% | nd | 81% |
| Cutoff 6 | 190 | nd | 184 |
| Sens 6 | 5% | nd | 7% |
| Spec 6 | 90% | nd | 90% |
| OR Quart 2 | 4.3 | nd | 13 |
| p Value | 0.11 | nd | 0.033 |
| 95% CI of | 0.71 | nd | 1.2 |
| OR Quart 2 | 27 | nd | 140 |
| OR Quart 3 | 7.4 | nd | 7.9 |
| p Value | 0.029 | nd | 0.085 |
| 95% CI of | 1.2 | nd | 0.75 |
| OR Quart 3 | 45 | nd | 82 |
| OR Quart 4 | 1.6 | nd | 4.1 |
| p Value | 0.63 | nd | 0.25 |
| 95% CI of | 0.23 | nd | 0.36 |
| OR Quart 4 | 11 | nd | 47 |

TABLE 8
Comparison of the maximum marker levels in EDTA samples
collected from Cohort 1 (patients that did not progress beyond RIFLE stage 0) and the
maximum values in EDTA samples collected from subjects between enrollment and 0, 24
hours, and 48 hours prior to reaching stage F in Cohort 2.
C-C motif chemokine 18

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 170 | 303 | 170 | 303 | nd | nd |
| Average | 230 | 472 | 230 | 463 | nd | nd |
| Stdev | 178 | 512 | 178 | 518 | nd | nd |
| p (t-test) | | 0.0041 | | 0.0058 | nd | nd |
| Min | 38.6 | 103 | 38.6 | 103 | nd | nd |
| Max | 1020 | 1650 | 1020 | 1650 | nd | nd |
| n (Samp) | 87 | 8 | 87 | 8 | nd | nd |
| n (Patient) | 87 | 8 | 87 | 8 | nd | nd |
| UO only | | | | | | |
| Median | 173 | 380 | 173 | 380 | nd | nd |
| Average | 247 | 580 | 247 | 580 | nd | nd |
| Stdev | 181 | 557 | 181 | 557 | nd | nd |
| p (t-test) | | 6.5E−4 | | 6.5E−4 | nd | nd |
| Min | 38.6 | 118 | 38.6 | 118 | nd | nd |
| Max | 848 | 1650 | 848 | 1650 | nd | nd |
| n (Samp) | 80 | 6 | 80 | 6 | nd | nd |
| n (Patient) | 80 | 6 | 80 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.68 | nd | 0.75 | 0.65 | nd | 0.75 | nd | nd | nd |
| SE | 0.11 | nd | 0.12 | 0.11 | nd | 0.12 | nd | nd | nd |
| p | 0.10 | nd | 0.037 | 0.18 | nd | 0.037 | nd | nd | nd |
| nCohort 1 | 87 | nd | 80 | 87 | nd | 80 | nd | nd | nd |
| nCohort 2 | 8 | nd | 6 | 8 | nd | 6 | nd | nd | nd |
| Cutoff 1 | 186 | nd | 264 | 120 | nd | 264 | nd | nd | nd |
| Sens 1 | 75% | nd | 83% | 75% | nd | 83% | nd | nd | nd |
| Spec 1 | 56% | nd | 65% | 31% | nd | 65% | nd | nd | nd |
| Cutoff 2 | 118 | nd | 264 | 118 | nd | 264 | nd | nd | nd |
| Sens 2 | 88% | nd | 83% | 88% | nd | 83% | nd | nd | nd |
| Spec 2 | 30% | nd | 65% | 30% | nd | 65% | nd | nd | nd |
| Cutoff 3 | 103 | nd | 117 | 103 | nd | 117 | nd | nd | nd |
| Sens 3 | 100% | nd | 100% | 100% | nd | 100% | nd | nd | nd |
| Spec 3 | 21% | nd | 25% | 21% | nd | 25% | nd | nd | nd |
| Cutoff 4 | 246 | nd | 283 | 246 | nd | 283 | nd | nd | nd |
| Sens 4 | 62% | nd | 67% | 62% | nd | 67% | nd | nd | nd |
| Spec 4 | 70% | nd | 70% | 70% | nd | 70% | nd | nd | nd |
| Cutoff 5 | 309 | nd | 379 | 309 | nd | 379 | nd | nd | nd |
| Sens 5 | 50% | nd | 50% | 50% | nd | 50% | nd | nd | nd |
| Spec 5 | 80% | nd | 80% | 80% | nd | 80% | nd | nd | nd |
| Cutoff 6 | 484 | nd | 507 | 484 | nd | 507 | nd | nd | nd |
| Sens 6 | 25% | nd | 33% | 25% | nd | 33% | nd | nd | nd |
| Spec 6 | 91% | nd | 90% | 91% | nd | 90% | nd | nd | nd |
| OR Quart 2 | 0.96 | nd | 0 | 2.0 | nd | 0 | nd | nd | nd |
| p Value | 0.98 | nd | na | 0.58 | nd | na | nd | nd | nd |
| 95% CI of | 0.056 | nd | na | 0.17 | nd | na | nd | nd | nd |
| OR Quart 2 | 16 | nd | na | 24 | nd | na | nd | nd | nd |
| OR Quart 3 | 2.0 | nd | 1.0 | 0.96 | nd | 1.0 | nd | nd | nd |
| p Value | 0.58 | nd | 1.0 | 0.98 | nd | 1.0 | nd | nd | nd |
| 95% CI of | 0.17 | nd | 0.058 | 0.056 | nd | 0.058 | nd | nd | nd |
| OR Quart 3 | 24 | nd | 17 | 16 | nd | 17 | nd | nd | nd |
| OR Quart 4 | 4.4 | nd | 4.4 | 4.4 | nd | 4.4 | nd | nd | nd |
| p Value | 0.20 | nd | 0.20 | 0.20 | nd | 0.20 | nd | nd | nd |
| 95% CI of | 0.45 | nd | 0.45 | 0.45 | nd | 0.45 | nd | nd | nd |
| OR Quart 4 | 43 | nd | 44 | 43 | nd | 44 | nd | nd | nd |

C-C motif chemokine 24

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 222 | 259 | 222 | 259 | 222 | 155 |
| Average | 400 | 476 | 400 | 366 | 400 | 299 |
| Stdev | 493 | 643 | 493 | 299 | 493 | 235 |
| p (t-test) | | 0.64 | | 0.82 | | 0.60 |
| Min | 0.0260 | 86.4 | 0.0260 | 86.4 | 0.0260 | 86.4 |
| Max | 2920 | 2430 | 2920 | 1100 | 2920 | 614 |
| n (Samp) | 64 | 12 | 64 | 12 | 64 | 7 |
| n (Patient) | 64 | 12 | 64 | 12 | 64 | 7 |
| sCr only | | | | | | |
| Median | 245 | 554 | 245 | 554 | nd | nd |
| Average | 386 | 755 | 386 | 534 | nd | nd |
| Stdev | 423 | 843 | 423 | 341 | nd | nd |
| p (t-test) | | 0.049 | | 0.40 | nd | nd |
| Min | 0.0260 | 95.2 | 0.0260 | 95.2 | nd | nd |
| Max | 2920 | 2430 | 2920 | 1100 | nd | nd |
| n (Samp) | 131 | 6 | 131 | 6 | nd | nd |
| n (Patient) | 131 | 6 | 131 | 6 | nd | nd |
| UO only | | | | | | |
| Median | 282 | 163 | 282 | 163 | 282 | 148 |
| Average | 467 | 237 | 467 | 237 | 467 | 249 |
| Stdev | 499 | 182 | 499 | 182 | 499 | 213 |
| p (t-test) | | 0.20 | | 0.20 | | 0.30 |
| Min | 24.7 | 86.4 | 24.7 | 86.4 | 24.7 | 86.4 |
| Max | 2920 | 614 | 2920 | 614 | 2920 | 614 |
| n (Samp) | 63 | 8 | 63 | 8 | 63 | 6 |
| n (Patient) | 63 | 8 | 63 | 8 | 63 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.54 | 0.68 | 0.35 | 0.54 | 0.67 | 0.35 | 0.48 | nd | 0.35 |
| SE | 0.093 | 0.12 | 0.11 | 0.093 | 0.12 | 0.11 | 0.12 | nd | 0.13 |
| p | 0.63 | 0.14 | 0.17 | 0.66 | 0.16 | 0.17 | 0.85 | nd | 0.23 |
| nCohort 1 | 64 | 131 | 63 | 64 | 131 | 63 | 64 | nd | 63 |
| nCohort 2 | 12 | 6 | 8 | 12 | 6 | 8 | 7 | nd | 6 |
| Cutoff 1 | 154 | 282 | 138 | 154 | 282 | 138 | 134 | nd | 89.8 |
| Sens 1 | 75% | 83% | 75% | 75% | 83% | 75% | 71% | nd | 83% |
| Spec 1 | 36% | 56% | 22% | 36% | 56% | 22% | 30% | nd | 13% |
| Cutoff 2 | 134 | 282 | 89.8 | 134 | 282 | 89.8 | 94.1 | nd | 89.8 |
| Sens 2 | 83% | 83% | 88% | 83% | 83% | 88% | 86% | nd | 83% |
| Spec 2 | 30% | 56% | 13% | 30% | 56% | 13% | 22% | nd | 13% |
| Cutoff 3 | 94.1 | 95.2 | 85.2 | 94.1 | 95.2 | 85.2 | 85.2 | nd | 85.2 |
| Sens 3 | 92% | 100% | 100% | 92% | 100% | 100% | 100% | nd | 100% |
| Spec 3 | 22% | 22% | 11% | 22% | 22% | 11% | 17% | nd | 11% |
| Cutoff 4 | 400 | 463 | 567 | 400 | 463 | 567 | 400 | nd | 567 |
| Sens 4 | 42% | 67% | 12% | 42% | 67% | 12% | 43% | nd | 17% |
| Spec 4 | 70% | 70% | 71% | 70% | 70% | 71% | 70% | nd | 71% |
| Cutoff 5 | 657 | 625 | 715 | 657 | 625 | 715 | 657 | nd | 715 |
| Sens 5 | 8% | 17% | 0% | 8% | 17% | 0% | 0% | nd | 0% |
| Spec 5 | 81% | 80% | 81% | 81% | 80% | 81% | 81% | nd | 81% |
| Cutoff 6 | 855 | 859 | 991 | 855 | 859 | 991 | 855 | nd | 991 |
| Sens 6 | 8% | 17% | 0% | 8% | 17% | 0% | 0% | nd | 0% |
| Spec 6 | 91% | 90% | 90% | 91% | 90% | 90% | 91% | nd | 90% |
| OR Quart 2 | 1.6 | 0 | 1.0 | 1.6 | 0 | 1.0 | 0.47 | nd | 1.1 |
| p Value | 0.63 | na | 1.0 | 0.63 | na | 1.0 | 0.55 | nd | 0.97 |
| 95% CI of | 0.23 | na | 0.058 | 0.23 | na | 0.058 | 0.039 | nd | 0.061 |
| OR Quart 2 | 11 | na | 17 | 11 | na | 17 | 5.7 | nd | 18 |
| OR Quart 3 | 2.3 | 2.1 | 3.4 | 2.3 | 2.1 | 3.4 | 1.0 | nd | 1.1 |
| p Value | 0.38 | 0.56 | 0.31 | 0.38 | 0.56 | 0.31 | 1.0 | nd | 0.97 |
| 95% CI of | 0.36 | 0.18 | 0.32 | 0.36 | 0.18 | 0.32 | 0.13 | nd | 0.061 |
| OR Quart 3 | 14 | 24 | 36 | 14 | 24 | 36 | 8.0 | nd | 18 |
| OR Quart 4 | 1.6 | 3.1 | 3.6 | 1.6 | 3.1 | 3.6 | 1.1 | nd | 3.6 |
| p Value | 0.63 | 0.34 | 0.29 | 0.63 | 0.34 | 0.29 | 0.95 | nd | 0.29 |
| 95% CI of | 0.23 | 0.31 | 0.34 | 0.23 | 0.31 | 0.34 | 0.13 | nd | 0.34 |
| OR Quart 4 | 11 | 31 | 39 | 11 | 31 | 39 | 8.6 | nd | 39 |

C-C motif chemokine 8

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 13.9 | 21.7 | 13.9 | 21.7 | 13.9 | 23.8 |
| Average | 20.1 | 42.4 | 20.1 | 42.3 | 20.1 | 37.2 |
| Stdev | 26.8 | 48.6 | 26.8 | 48.7 | 26.8 | 35.5 |
| p (t-test) | | 0.025 | | 0.026 | | 0.13 |
| Min | 1.20 | 6.24 | 1.20 | 6.24 | 1.20 | 6.24 |
| Max | 148 | 169 | 148 | 169 | 148 | 101 |
| n (Samp) | 64 | 12 | 64 | 12 | 64 | 7 |
| n (Patient) | 64 | 12 | 64 | 12 | 64 | 7 |
| sCr only | | | | | | |
| Median | 15.1 | 21.8 | 15.1 | 21.1 | nd | nd |
| Average | 20.2 | 55.5 | 20.2 | 55.3 | nd | nd |
| Stdev | 25.1 | 65.4 | 25.1 | 65.6 | nd | nd |
| p (t-test) | | 0.0027 | | 0.0028 | nd | nd |
| Min | 1.20 | 6.24 | 1.20 | 6.24 | nd | nd |
| Max | 180 | 169 | 180 | 169 | nd | nd |
| n (Samp) | 131 | 6 | 131 | 6 | nd | nd |
| n (Patient) | 131 | 6 | 131 | 6 | nd | nd |
| UO only | | | | | | |
| Median | 13.7 | 21.7 | 13.7 | 21.7 | 13.7 | 20.4 |
| Average | 21.2 | 26.3 | 21.2 | 26.3 | 21.2 | 26.5 |
| Stdev | 27.6 | 20.6 | 27.6 | 20.6 | 27.6 | 23.7 |
| p (t-test) | | 0.61 | | 0.61 | | 0.65 |
| Min | 1.20 | 6.24 | 1.20 | 6.24 | 1.20 | 6.24 |
| Max | 148 | 72.1 | 148 | 72.1 | 148 | 72.1 |
| n (Samp) | 63 | 8 | 63 | 8 | 63 | 6 |
| n (Patient) | 63 | 8 | 63 | 8 | 63 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.70 | 0.66 | 0.66 | 0.70 | 0.65 | 0.66 | 0.69 | nd | 0.63 |
| SE | 0.090 | 0.12 | 0.11 | 0.090 | 0.12 | 0.11 | 0.12 | nd | 0.13 |
| p | 0.025 | 0.21 | 0.15 | 0.030 | 0.24 | 0.15 | 0.097 | nd | 0.29 |
| nCohort 1 | 64 | 131 | 63 | 64 | 131 | 63 | 64 | nd | 63 |
| nCohort 2 | 12 | 6 | 8 | 12 | 6 | 8 | 7 | nd | 6 |
| Cutoff 1 | 14.2 | 13.4 | 14.2 | 13.7 | 13.4 | 14.2 | 16.9 | nd | 11.3 |
| Sens 1 | 75% | 83% | 75% | 75% | 83% | 75% | 71% | nd | 83% |
| Spec 1 | 52% | 44% | 54% | 50% | 44% | 54% | 64% | nd | 41% |
| Cutoff 2 | 13.0 | 13.4 | 11.3 | 13.0 | 13.4 | 11.3 | 11.3 | nd | 11.3 |
| Sens 2 | 83% | 83% | 88% | 83% | 83% | 88% | 86% | nd | 83% |
| Spec 2 | 48% | 44% | 41% | 48% | 44% | 41% | 36% | nd | 41% |
| Cutoff 3 | 11.3 | 5.84 | 4.81 | 11.3 | 5.84 | 4.81 | 4.81 | nd | 4.81 |
| Sens 3 | 92% | 100% | 100% | 92% | 100% | 100% | 100% | nd | 100% |
| Spec 3 | 36% | 13% | 14% | 36% | 13% | 14% | 16% | nd | 14% |
| Cutoff 4 | 18.9 | 18.9 | 20.1 | 18.9 | 18.9 | 20.1 | 18.9 | nd | 20.1 |
| Sens 4 | 58% | 50% | 50% | 58% | 50% | 50% | 57% | nd | 50% |
| Spec 4 | 70% | 70% | 73% | 70% | 70% | 73% | 70% | nd | 73% |
| Cutoff 5 | 22.4 | 22.1 | 24.3 | 22.4 | 22.1 | 24.3 | 22.4 | nd | 24.3 |
| Sens 5 | 50% | 50% | 38% | 50% | 50% | 38% | 57% | nd | 33% |
| Spec 5 | 81% | 80% | 81% | 81% | 80% | 81% | 81% | nd | 81% |
| Cutoff 6 | 30.2 | 30.5 | 39.5 | 30.2 | 30.5 | 39.5 | 30.2 | nd | 39.5 |
| Sens 6 | 33% | 33% | 12% | 33% | 33% | 12% | 29% | nd | 17% |
| Spec 6 | 91% | 90% | 92% | 91% | 90% | 92% | 91% | nd | 92% |
| OR Quart 2 | 3.4 | 2.1 | 0.94 | 3.4 | 2.1 | 0.94 | 0.94 | nd | 1.0 |
| p Value | 0.31 | 0.56 | 0.97 | 0.31 | 0.56 | 0.97 | 0.97 | nd | 1.0 |
| 95% CI of | 0.32 | 0.18 | 0.054 | 0.32 | 0.18 | 0.054 | 0.054 | nd | 0.057 |
| OR Quart 2 | 36 | 24 | 16 | 36 | 24 | 16 | 16 | nd | 17 |
| OR Quart 3 | 2.1 | 0 | 2.0 | 2.1 | 0 | 2.0 | 0.94 | nd | 1.0 |
| p Value | 0.55 | na | 0.59 | 0.55 | na | 0.59 | 0.97 | nd | 1.0 |
| 95% CI of | 0.18 | na | 0.16 | 0.18 | na | 0.16 | 0.054 | nd | 0.057 |
| OR Quart 3 | 26 | na | 24 | 26 | na | 24 | 16 | nd | 17 |
| OR Quart 4 | 8.3 | 3.1 | 4.6 | 8.3 | 3.1 | 4.6 | 4.6 | nd | 3.2 |
| p Value | 0.063 | 0.34 | 0.20 | 0.063 | 0.34 | 0.20 | 0.20 | nd | 0.34 |
| 95% CI of | 0.89 | 0.31 | 0.46 | 0.89 | 0.31 | 0.46 | 0.46 | nd | 0.30 |
| OR Quart 4 | 78 | 31 | 46 | 78 | 31 | 46 | 46 | nd | 34 |

Cathepsin D

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 233000 | 479000 | 233000 | 479000 | nd | nd |
| Average | 256000 | 538000 | 256000 | 452000 | nd | nd |
| Stdev | 149000 | 348000 | 149000 | 144000 | nd | nd |
| p (t-test) | | 2.6E−5 | | 5.9E−4 | nd | nd |
| Min | 34100 | 266000 | 34100 | 266000 | nd | nd |
| Max | 1020000 | 1350000 | 1020000 | 655000 | nd | nd |
| n (Samp) | 86 | 8 | 86 | 8 | nd | nd |
| n (Patient) | 86 | 8 | 86 | 8 | nd | nd |
| UO only | | | | | | |
| Median | 247000 | 479000 | 247000 | 479000 | nd | nd |
| Average | 278000 | 442000 | 278000 | 442000 | nd | nd |
| Stdev | 153000 | 128000 | 153000 | 128000 | nd | nd |
| p (t-test) | | 0.012 | | 0.012 | nd | nd |
| Min | 69400 | 266000 | 69400 | 266000 | nd | nd |
| Max | 1020000 | 561000 | 1020000 | 561000 | nd | nd |
| n (Samp) | 79 | 6 | 79 | 6 | nd | nd |
| n (Patient) | 79 | 6 | 79 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.86 | nd | 0.83 | 0.86 | nd | 0.83 | nd | nd | nd |
| SE | 0.084 | nd | 0.10 | 0.084 | nd | 0.10 | nd | nd | nd |
| p | 1.3E−5 | nd | 0.0016 | 1.8E−5 | nd | 0.0016 | nd | nd | nd |
| nCohort 1 | 86 | nd | 79 | 86 | nd | 79 | nd | nd | nd |
| nCohort 2 | 8 | nd | 6 | 8 | nd | 6 | nd | nd | nd |
| Cutoff 1 | 307000 | nd | 307000 | 307000 | nd | 307000 | nd | nd | nd |
| Sens 1 | 75% | nd | 83% | 75% | nd | 83% | nd | nd | nd |
| Spec 1 | 74% | nd | 67% | 74% | nd | 67% | nd | nd | nd |
| Cutoff 2 | 307000 | nd | 307000 | 307000 | nd | 307000 | nd | nd | nd |
| Sens 2 | 88% | nd | 83% | 88% | nd | 83% | nd | nd | nd |
| Spec 2 | 73% | nd | 67% | 73% | nd | 67% | nd | nd | nd |
| Cutoff 3 | 263000 | nd | 263000 | 263000 | nd | 263000 | nd | nd | nd |
| Sens 3 | 100% | nd | 100% | 100% | nd | 100% | nd | nd | nd |
| Spec 3 | 66% | nd | 58% | 66% | nd | 58% | nd | nd | nd |
| Cutoff 4 | 302000 | nd | 310000 | 302000 | nd | 310000 | nd | nd | nd |
| Sens 4 | 88% | nd | 67% | 88% | nd | 67% | nd | nd | nd |
| Spec 4 | 71% | nd | 71% | 71% | nd | 71% | nd | nd | nd |
| Cutoff 5 | 324000 | nd | 374000 | 324000 | nd | 374000 | nd | nd | nd |
| Sens 5 | 62% | nd | 67% | 62% | nd | 67% | nd | nd | nd |
| Spec 5 | 80% | nd | 81% | 80% | nd | 81% | nd | nd | nd |
| Cutoff 6 | 442000 | nd | 471000 | 442000 | nd | 471000 | nd | nd | nd |
| Sens 6 | 62% | nd | 50% | 62% | nd | 50% | nd | nd | nd |
| Spec 6 | 91% | nd | 91% | 91% | nd | 91% | nd | nd | nd |
| OR Quart 2 | >0 | nd | >0 | >0 | nd | >0 | nd | nd | nd |
| p Value | <na | nd | <na | <na | nd | <na | nd | nd | nd |
| 95% CI of | >na | nd | >na | >na | nd | >na | nd | nd | nd |
| OR Quart 2 | na | nd | na | na | nd | na | nd | nd | nd |
| OR Quart 3 | >3.4 | nd | >2.2 | >3.4 | nd | >2.2 | nd | nd | nd |
| p Value | <0.30 | nd | <0.53 | <0.30 | nd | <0.53 | nd | nd | nd |
| 95% CI of | >0.33 | nd | >0.19 | >0.33 | nd | >0.19 | nd | nd | nd |
| OR Quart 3 | na | nd | na | na | nd | na | nd | nd | nd |
| OR Quart 4 | >6.1 | nd | >4.7 | >6.1 | nd | >4.7 | nd | nd | nd |
| p Value | <0.11 | nd | <0.19 | <0.11 | nd | <0.19 | nd | nd | nd |
| 95% CI of | >0.65 | nd | >0.48 | >0.65 | nd | >0.48 | nd | nd | nd |
| OR Quart 4 | na | nd | na | na | nd | na | nd | nd | nd |

C—X—C motif chemokine 13

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 47.3 | 83.0 | 47.3 | 83.0 | 47.3 | 82.8 |
| Average | 162 | 306 | 162 | 306 | 162 | 190 |
| Stdev | 351 | 564 | 351 | 564 | 351 | 234 |
| p (t-test) | | 0.24 | | 0.24 | | 0.84 |
| Min | 9.90 | 15.1 | 9.90 | 15.1 | 9.90 | 45.0 |
| Max | 1790 | 2000 | 1790 | 2000 | 1790 | 685 |
| n (Samp) | 64 | 12 | 64 | 12 | 64 | 7 |
| n (Patient) | 64 | 12 | 64 | 12 | 64 | 7 |
| sCr only | | | | | | |
| Median | 53.6 | 83.0 | 53.6 | 83.0 | nd | nd |
| Average | 150 | 399 | 150 | 399 | nd | nd |
| Stdev | 294 | 785 | 294 | 785 | nd | nd |
| p (t-test) | | 0.070 | | 0.070 | nd | nd |
| Min | 9.90 | 15.1 | 9.90 | 15.1 | nd | nd |
| Max | 1790 | 2000 | 1790 | 2000 | nd | nd |
| n (Samp) | 131 | 6 | 131 | 6 | nd | nd |
| n (Patient) | 131 | 6 | 131 | 6 | nd | nd |
| UO only | | | | | | |
| Median | 51.9 | 77.2 | 51.9 | 77.2 | 51.9 | 77.2 |
| Average | 149 | 180 | 149 | 180 | 149 | 208 |
| Stdev | 298 | 221 | 298 | 221 | 298 | 251 |
| p (t-test) | | 0.78 | | 0.78 | | 0.64 |
| Min | 9.90 | 35.6 | 9.90 | 35.6 | 9.90 | 45.0 |
| Max | 1790 | 685 | 1790 | 685 | 1790 | 685 |
| n (Samp) | 63 | 8 | 63 | 8 | 63 | 6 |
| n (Patient) | 63 | 8 | 63 | 8 | 63 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.65 | 0.60 | 0.64 | 0.65 | 0.60 | 0.64 | 0.67 | nd | 0.66 |
| SE | 0.092 | 0.12 | 0.11 | 0.092 | 0.12 | 0.11 | 0.12 | nd | 0.13 |
| p | 0.11 | 0.45 | 0.22 | 0.11 | 0.45 | 0.22 | 0.15 | nd | 0.20 |
| nCohort 1 | 64 | 131 | 63 | 64 | 131 | 63 | 64 | nd | 63 |
| nCohort 2 | 12 | 6 | 8 | 12 | 6 | 8 | 7 | nd | 6 |
| Cutoff 1 | 66.4 | 70.0 | 66.4 | 66.4 | 70.0 | 66.4 | 68.9 | nd | 66.4 |
| Sens 1 | 75% | 83% | 75% | 75% | 83% | 75% | 71% | nd | 83% |
| Spec 1 | 58% | 56% | 54% | 58% | 56% | 54% | 58% | nd | 54% |
| Cutoff 2 | 44.3 | 70.0 | 44.3 | 44.3 | 70.0 | 44.3 | 66.4 | nd | 66.4 |
| Sens 2 | 83% | 83% | 88% | 83% | 83% | 88% | 86% | nd | 83% |
| Spec 2 | 45% | 56% | 41% | 45% | 56% | 41% | 58% | nd | 54% |
| Cutoff 3 | 35.5 | 15.0 | 35.5 | 35.5 | 15.0 | 35.5 | 44.3 | nd | 44.3 |
| Sens 3 | 92% | 100% | 100% | 92% | 100% | 100% | 100% | nd | 100% |
| Spec 3 | 39% | 5% | 32% | 39% | 5% | 32% | 45% | nd | 41% |
| Cutoff 4 | 106 | 114 | 111 | 106 | 114 | 111 | 106 | nd | 111 |
| Sens 4 | 42% | 33% | 38% | 42% | 33% | 38% | 29% | nd | 33% |
| Spec 4 | 70% | 70% | 71% | 70% | 70% | 71% | 70% | nd | 71% |
| Cutoff 5 | 170 | 155 | 140 | 170 | 155 | 140 | 170 | nd | 140 |
| Sens 5 | 25% | 17% | 38% | 25% | 17% | 38% | 29% | nd | 33% |
| Spec 5 | 81% | 80% | 81% | 81% | 80% | 81% | 81% | nd | 81% |
| Cutoff 6 | 254 | 260 | 254 | 254 | 260 | 254 | 254 | nd | 254 |
| Sens 6 | 25% | 17% | 25% | 25% | 17% | 25% | 29% | nd | 33% |
| Spec 6 | 91% | 90% | 90% | 91% | 90% | 90% | 91% | nd | 90% |
| OR Quart 2 | 2.1 | 0 | >2.1 | 2.1 | 0 | >2.1 | >1.0 | nd | >1.1 |
| p Value | 0.55 | na | <0.55 | 0.55 | na | <0.55 | <1.0 | nd | <0.97 |
| 95% CI of | 0.18 | na | >0.18 | 0.18 | na | >0.18 | >0.058 | nd | >0.061 |
| OR Quart 2 | 26 | na | na | 26 | na | na | na | nd | na |
| OR Quart 3 | 4.8 | 3.2 | >3.4 | 4.8 | 3.2 | >3.4 | >4.9 | nd | >3.6 |
| p Value | 0.18 | 0.33 | <0.31 | 0.18 | 0.33 | <0.31 | <0.18 | nd | <0.29 |
| 95% CI of | 0.48 | 0.32 | >0.32 | 0.48 | 0.32 | >0.32 | >0.49 | nd | >0.34 |
| OR Quart 3 | 48 | 32 | na | 48 | 32 | na | na | nd | na |
| OR Quart 4 | 6.4 | 2.0 | >3.4 | 6.4 | 2.0 | >3.4 | >2.1 | nd | >2.1 |
| p Value | 0.11 | 0.58 | <0.31 | 0.11 | 0.58 | <0.31 | <0.55 | nd | <0.55 |
| 95% CI of | 0.67 | 0.17 | >0.32 | 0.67 | 0.17 | >0.32 | >0.18 | nd | >0.18 |
| OR Quart 4 | 61 | 23 | na | 61 | 23 | na | na | nd | na |

Insulin-like growth factor-binding protein 3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3530 | 4230 | 3530 | 3620 | nd | nd |
| Average | 3640 | 4460 | 3640 | 4160 | nd | nd |
| Stdev | 1520 | 1990 | 1520 | 1840 | nd | nd |
| p (t-test) | | 0.16 | | 0.36 | nd | nd |
| Min | 823 | 2070 | 823 | 2070 | nd | nd |
| Max | 8260 | 7360 | 8260 | 7360 | nd | nd |
| n (Samp) | 87 | 8 | 87 | 8 | nd | nd |
| n (Patient) | 87 | 8 | 87 | 8 | nd | nd |
| UO only | | | | | | |
| Median | 3370 | 4230 | 3370 | 4230 | nd | nd |
| Average | 3670 | 4490 | 3670 | 4490 | nd | nd |
| Stdev | 1550 | 2000 | 1550 | 2000 | nd | nd |
| p (t-test) | | 0.22 | | 0.22 | nd | nd |
| Min | 823 | 2070 | 823 | 2070 | nd | nd |
| Max | 8260 | 7360 | 8260 | 7360 | nd | nd |
| n (Samp) | 80 | 6 | 80 | 6 | nd | nd |
| n (Patient) | 80 | 6 | 80 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.63 | nd | 0.64 | 0.59 | nd | 0.64 | nd | nd | nd |
| SE | 0.11 | nd | 0.13 | 0.11 | nd | 0.13 | nd | nd | nd |
| p | 0.23 | nd | 0.25 | 0.43 | nd | 0.25 | nd | nd | nd |
| nCohort 1 | 87 | nd | 80 | 87 | nd | 80 | nd | nd | nd |
| nCohort 2 | 8 | nd | 6 | 8 | nd | 6 | nd | nd | nd |
| Cutoff 1 | 3050 | nd | 3090 | 3050 | nd | 3090 | nd | nd | nd |
| Sens 1 | 75% | nd | 83% | 75% | nd | 83% | nd | nd | nd |
| Spec 1 | 46% | nd | 48% | 46% | nd | 48% | nd | nd | nd |
| Cutoff 2 | 2400 | nd | 3090 | 2400 | nd | 3090 | nd | nd | nd |
| Sens 2 | 88% | nd | 83% | 88% | nd | 83% | nd | nd | nd |
| Spec 2 | 22% | nd | 48% | 22% | nd | 48% | nd | nd | nd |
| Cutoff 3 | 2040 | nd | 2040 | 2040 | nd | 2040 | nd | nd | nd |
| Sens 3 | 100% | nd | 100% | 100% | nd | 100% | nd | nd | nd |
| Spec 3 | 16% | nd | 15% | 16% | nd | 15% | nd | nd | nd |
| Cutoff 4 | 4330 | nd | 4220 | 4330 | nd | 4220 | nd | nd | nd |
| Sens 4 | 50% | nd | 50% | 38% | nd | 50% | nd | nd | nd |
| Spec 4 | 70% | nd | 70% | 70% | nd | 70% | nd | nd | nd |
| Cutoff 5 | 4990 | nd | 4990 | 4990 | nd | 4990 | nd | nd | nd |
| Sens 5 | 50% | nd | 50% | 38% | nd | 50% | nd | nd | nd |
| Spec 5 | 80% | nd | 80% | 80% | nd | 80% | nd | nd | nd |
| Cutoff 6 | 5650 | nd | 5590 | 5650 | nd | 5590 | nd | nd | nd |
| Sens 6 | 38% | nd | 33% | 25% | nd | 33% | nd | nd | nd |
| Spec 6 | 91% | nd | 90% | 91% | nd | 90% | nd | nd | nd |
| OR Quart 2 | 0.95 | nd | 2.0 | 0.95 | nd | 2.0 | nd | nd | nd |
| p Value | 0.96 | nd | 0.58 | 0.96 | nd | 0.58 | nd | nd | nd |
| 95% CI of | 0.12 | nd | 0.17 | 0.12 | nd | 0.17 | nd | nd | nd |
| OR Quart 2 | 7.4 | nd | 24 | 7.4 | nd | 24 | nd | nd | nd |
| OR Quart 3 | 0 | nd | 0 | 0.46 | nd | 0 | nd | nd | nd |
| p Value | na | nd | na | 0.53 | nd | na | nd | nd | nd |
| 95% CI of | na | nd | na | 0.039 | nd | na | nd | nd | nd |
| OR Quart 3 | na | nd | na | 5.4 | nd | na | nd | nd | nd |
| OR Quart 4 | 2.1 | nd | 3.2 | 1.5 | nd | 3.2 | nd | nd | nd |
| p Value | 0.42 | nd | 0.34 | 0.67 | nd | 0.34 | nd | nd | nd |
| 95% CI of | 0.35 | nd | 0.30 | 0.23 | nd | 0.30 | nd | nd | nd |
| OR Quart 4 | 13 | nd | 33 | 9.9 | nd | 33 | nd | nd | nd |

Interleukin-11

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 98.9 | 101 | 98.9 | 101 | 98.9 | 90.7 |
| Average | 333 | 576 | 333 | 576 | 333 | 129 |
| Stdev | 1130 | 1530 | 1130 | 1530 | 1130 | 76.7 |
| p (t-test) | | 0.52 | | 0.52 | | 0.64 |
| Min | 0.368 | 31.8 | 0.368 | 31.8 | 0.368 | 38.4 |
| Max | 6920 | 5420 | 6920 | 5420 | 6920 | 233 |
| n (Samp) | 65 | 12 | 65 | 12 | 65 | 7 |
| n (Patient) | 65 | 12 | 65 | 12 | 65 | 7 |
| sCr only | | | | | | |
| Median | 91.9 | 83.6 | 91.9 | 83.6 | nd | nd |
| Average | 218 | 143 | 218 | 143 | nd | nd |
| Stdev | 804 | 136 | 804 | 136 | nd | nd |
| p (t-test) | | 0.82 | | 0.82 | nd | nd |
| Min | 0.359 | 31.8 | 0.359 | 31.8 | nd | nd |
| Max | 6920 | 389 | 6920 | 389 | nd | nd |
| n (Samp) | 132 | 6 | 132 | 6 | nd | nd |
| n (Patient) | 132 | 6 | 132 | 6 | nd | nd |
| UO only | | | | | | |
| Median | 90.7 | 101 | 90.7 | 101 | 90.7 | 83.7 |
| Average | 319 | 778 | 319 | 778 | 319 | 114 |
| Stdev | 1140 | 1880 | 1140 | 1880 | 1140 | 72.9 |
| p (t-test) | | 0.33 | | 0.33 | | 0.67 |
| Min | 0.368 | 51.1 | 0.368 | 51.1 | 0.368 | 38.4 |
| Max | 6920 | 5420 | 6920 | 5420 | 6920 | 233 |
| n (Samp) | 64 | 8 | 64 | 8 | 64 | 6 |
| n (Patient) | 64 | 8 | 64 | 8 | 64 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.59 | 0.56 | 0.63 | 0.59 | 0.56 | 0.63 | 0.57 | nd | 0.57 |
| SE | 0.093 | 0.12 | 0.11 | 0.093 | 0.12 | 0.11 | 0.12 | nd | 0.13 |
| p | 0.31 | 0.63 | 0.24 | 0.31 | 0.63 | 0.24 | 0.55 | nd | 0.59 |
| nCohort 1 | 65 | 132 | 64 | 65 | 132 | 64 | 65 | nd | 64 |
| nCohort 2 | 12 | 6 | 8 | 12 | 6 | 8 | 7 | nd | 6 |
| Cutoff 1 | 67.2 | 51.1 | 76.5 | 67.2 | 51.1 | 76.5 | 76.5 | nd | 71.6 |
| Sens 1 | 75% | 83% | 75% | 75% | 83% | 75% | 71% | nd | 83% |
| Spec 1 | 43% | 39% | 47% | 43% | 39% | 47% | 43% | nd | 47% |
| Cutoff 2 | 51.1 | 51.1 | 71.6 | 51.1 | 51.1 | 71.6 | 67.2 | nd | 71.6 |
| Sens 2 | 83% | 83% | 88% | 83% | 83% | 88% | 86% | nd | 83% |
| Spec 2 | 38% | 39% | 47% | 38% | 39% | 47% | 43% | nd | 47% |
| Cutoff 3 | 47.4 | 29.7 | 47.4 | 47.4 | 29.7 | 47.4 | 33.9 | nd | 33.9 |
| Sens 3 | 92% | 100% | 100% | 92% | 100% | 100% | 100% | nd | 100% |
| Spec 3 | 38% | 23% | 41% | 38% | 23% | 41% | 32% | nd | 33% |
| Cutoff 4 | 159 | 158 | 155 | 159 | 158 | 155 | 159 | nd | 155 |
| Sens 4 | 42% | 33% | 38% | 42% | 33% | 38% | 43% | nd | 33% |
| Spec 4 | 71% | 73% | 70% | 71% | 73% | 70% | 71% | nd | 70% |
| Cutoff 5 | 209 | 196 | 190 | 209 | 196 | 190 | 209 | nd | 190 |
| Sens 5 | 33% | 33% | 25% | 33% | 33% | 25% | 29% | nd | 17% |
| Spec 5 | 80% | 81% | 81% | 80% | 81% | 81% | 80% | nd | 81% |
| Cutoff 6 | 370 | 261 | 306 | 370 | 261 | 306 | 370 | nd | 306 |
| Sens 6 | 17% | 17% | 12% | 17% | 17% | 12% | 0% | nd | 0% |
| Spec 6 | 91% | 90% | 91% | 91% | 90% | 91% | 91% | nd | 91% |
| OR Quart 2 | >8.8 | >4.4 | >5.1 | >8.8 | >4.4 | >5.1 | >5.1 | nd | >3.4 |
| p Value | <0.057 | <0.20 | <0.16 | <0.057 | <0.20 | <0.16 | <0.16 | nd | <0.31 |
| 95% CI of | >0.94 | >0.46 | >0.52 | >0.94 | >0.46 | >0.52 | >0.52 | nd | >0.32 |
| OR Quart 2 | na | na | na | na | na | na | na | nd | na |
| OR Quart 3 | >2.2 | >0 | >2.2 | >2.2 | >0 | >2.2 | >1.1 | nd | >1.1 |
| p Value | <0.53 | <na | <0.52 | <0.53 | <na | <0.52 | <0.97 | nd | <0.97 |
| 95% CI of | >0.19 | >na | >0.19 | >0.19 | >na | >0.19 | >0.061 | nd | >0.061 |
| OR Quart 3 | na | na | na | na | na | na | na | nd | na |
| OR Quart 4 | >4.8 | >2.1 | >2.2 | >4.8 | >2.1 | >2.2 | >2.2 | nd | >2.1 |
| p Value | <0.18 | <0.56 | <0.52 | <0.18 | <0.56 | <0.52 | <0.52 | nd | <0.55 |
| 95% CI of | >0.48 | >0.18 | >0.19 | >0.48 | >0.18 | >0.19 | >0.19 | nd | >0.18 |
| OR Quart 4 | na | na | na | na | na | na | na | nd | na |

Interleukin-2 receptor alpha chain

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 22.9 | 93.0 | 22.9 | 85.8 | 22.9 | 89.7 |
| Average | 45.2 | 158 | 45.2 | 152 | 45.2 | 121 |
| Stdev | 56.9 | 163 | 56.9 | 166 | 56.9 | 114 |
| p (t-test) | | 3.7E−5 | | 9.1E−5 | | 0.0041 |
| Min | 0.0359 | 5.03 | 0.0359 | 5.03 | 0.0359 | 12.5 |
| Max | 232 | 533 | 232 | 533 | 232 | 352 |
| n (Samp) | 65 | 12 | 65 | 12 | 65 | 7 |
| n (Patient) | 65 | 12 | 65 | 12 | 65 | 7 |
| sCr only | | | | | | |
| Median | 28.1 | 72.0 | 28.1 | 47.9 | nd | nd |
| Average | 80.9 | 66.1 | 80.9 | 55.7 | nd | nd |
| Stdev | 146 | 51.1 | 146 | 50.1 | nd | nd |
| p (t-test) | | 0.81 | | 0.67 | nd | nd |
| Min | 0.0359 | 5.03 | 0.0359 | 5.03 | nd | nd |
| Max | 1040 | 139 | 1040 | 139 | nd | nd |
| n (Samp) | 132 | 6 | 132 | 6 | nd | nd |
| n (Patient) | 132 | 6 | 132 | 6 | nd | nd |
| UO only | | | | | | |
| Median | 38.4 | 122 | 38.4 | 122 | 38.4 | 75.9 |
| Average | 55.1 | 196 | 55.1 | 196 | 55.1 | 117 |
| Stdev | 59.7 | 188 | 59.7 | 188 | 59.7 | 125 |
| p (t-test) | | 1.9E−5 | | 1.9E−5 | | 0.032 |
| Min | 0.0359 | 12.5 | 0.0359 | 12.5 | 0.0359 | 12.5 |
| Max | 232 | 533 | 232 | 533 | 232 | 352 |
| n (Samp) | 64 | 8 | 64 | 8 | 64 | 6 |
| n (Patient) | 64 | 8 | 64 | 8 | 64 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.78 | 0.60 | 0.77 | 0.76 | 0.57 | 0.77 | 0.77 | nd | 0.69 |
| SE | 0.083 | 0.13 | 0.10 | 0.085 | 0.12 | 0.10 | 0.11 | nd | 0.12 |
| p | 8.5E−4 | 0.40 | 0.0085 | 0.0026 | 0.60 | 0.0085 | 0.013 | nd | 0.13 |
| nCohort 1 | 65 | 132 | 64 | 65 | 132 | 64 | 65 | nd | 64 |
| nCohort 2 | 12 | 6 | 8 | 12 | 6 | 8 | 7 | nd | 6 |
| Cutoff 1 | 61.5 | 10.6 | 61.5 | 33.6 | 10.6 | 61.5 | 61.5 | nd | 29.5 |
| Sens 1 | 75% | 83% | 75% | 75% | 83% | 75% | 71% | nd | 83% |
| Spec 1 | 74% | 36% | 66% | 57% | 36% | 66% | 74% | nd | 47% |
| Cutoff 2 | 29.5 | 10.6 | 29.5 | 29.5 | 10.6 | 29.5 | 29.5 | nd | 29.5 |
| Sens 2 | 83% | 83% | 88% | 83% | 83% | 88% | 86% | nd | 83% |
| Spec 2 | 57% | 36% | 47% | 57% | 36% | 47% | 57% | nd | 47% |
| Cutoff 3 | 10.6 | 4.75 | 10.6 | 10.6 | 4.75 | 10.6 | 10.6 | nd | 10.6 |
| Sens 3 | 92% | 100% | 100% | 92% | 100% | 100% | 100% | nd | 100% |
| Spec 3 | 42% | 28% | 33% | 42% | 28% | 33% | 42% | nd | 33% |
| Cutoff 4 | 51.4 | 68.3 | 74.5 | 51.4 | 68.3 | 74.5 | 51.4 | nd | 74.5 |
| Sens 4 | 75% | 50% | 62% | 67% | 33% | 62% | 71% | nd | 50% |
| Spec 4 | 71% | 70% | 70% | 71% | 70% | 70% | 71% | nd | 70% |
| Cutoff 5 | 89.4 | 132 | 105 | 89.4 | 132 | 105 | 89.4 | nd | 105 |
| Sens 5 | 58% | 17% | 50% | 50% | 17% | 50% | 57% | nd | 33% |
| Spec 5 | 80% | 80% | 81% | 80% | 80% | 81% | 80% | nd | 81% |
| Cutoff 6 | 133 | 203 | 148 | 133 | 203 | 148 | 133 | nd | 148 |
| Sens 6 | 42% | 0% | 50% | 42% | 0% | 50% | 43% | nd | 33% |
| Spec 6 | 91% | 90% | 91% | 91% | 90% | 91% | 91% | nd | 91% |
| OR Quart 2 | >2.2 | >2.1 | >2.2 | >2.2 | >2.1 | >2.2 | >1.1 | nd | >2.1 |
| p Value | <0.53 | <0.56 | <0.52 | <0.53 | <0.56 | <0.52 | <0.97 | nd | <0.55 |
| 95% CI of | >0.19 | >0.18 | >0.19 | >0.19 | >0.18 | >0.19 | >0.061 | nd | >0.18 |
| OR Quart 2 | na | na | na | na | na | na | na | nd | na |
| OR Quart 3 | >3.6 | >2.1 | >2.2 | >5.1 | >3.3 | >2.2 | >2.2 | nd | >1.1 |
| p Value | <0.29 | <0.55 | <0.52 | <0.17 | <0.31 | <0.52 | <0.52 | nd | <0.97 |
| 95% CI of | >0.34 | >0.18 | >0.19 | >0.51 | >0.32 | >0.19 | >0.19 | nd | >0.061 |
| OR Quart 3 | na | na | na | na | na | na | na | nd | na |
| OR Quart 4 | >10 | >2.1 | >5.1 | >8.1 | >1.0 | >5.1 | >5.1 | nd | >3.4 |
| p Value | <0.039 | <0.56 | <0.16 | <0.065 | <1.0 | <0.16 | <0.16 | nd | <0.31 |
| 95% CI of | >1.1 | >0.18 | >0.52 | >0.88 | >0.060 | >0.52 | >0.52 | nd | >0.32 |
| OR Quart 4 | na | na | na | na | na | na | na | nd | na |

Neutrophil collagenase

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1220 | 4220 | 1220 | 4220 | nd | nd |
| Average | 2280 | 11600 | 2280 | 11600 | nd | nd |
| Stdev | 2900 | 15500 | 2900 | 15500 | nd | nd |
| p (t-test) | | 3.1E−6 | | 3.2E−6 | nd | nd |
| Min | 131 | 21.8 | 131 | 1.14 | nd | nd |
| Max | 16000 | 38700 | 16000 | 38700 | nd | nd |
| n (Samp) | 87 | 8 | 87 | 8 | nd | nd |
| n (Patient) | 87 | 8 | 87 | 8 | nd | nd |
| UO only | | | | | | |
| Median | 1190 | 5900 | 1190 | 5900 | nd | nd |
| Average | 3030 | 14800 | 3030 | 14800 | nd | nd |
| Stdev | 6790 | 16900 | 6790 | 16900 | nd | nd |
| p (t-test) | | 5.5E−4 | | 5.5E−4 | nd | nd |
| Min | 131 | 1500 | 131 | 1500 | nd | nd |
| Max | 55000 | 38700 | 55000 | 38700 | nd | nd |
| n (Samp) | 80 | 6 | 80 | 6 | nd | nd |
| n (Patient) | 80 | 6 | 80 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.76 | nd | 0.86 | 0.76 | nd | 0.86 | nd | nd | nd |
| SE | 0.10 | nd | 0.098 | 0.10 | nd | 0.098 | nd | nd | nd |
| p | 0.012 | nd | 2.3E−4 | 0.012 | nd | 2.3E−4 | nd | nd | nd |
| nCohort 1 | 87 | nd | 80 | 87 | nd | 80 | nd | nd | nd |
| nCohort 2 | 8 | nd | 6 | 8 | nd | 6 | nd | nd | nd |
| Cutoff 1 | 2830 | nd | 2830 | 2830 | nd | 2830 | nd | nd | nd |
| Sens 1 | 75% | nd | 83% | 75% | nd | 83% | nd | nd | nd |
| Spec 1 | 77% | nd | 76% | 77% | nd | 76% | nd | nd | nd |
| Cutoff 2 | 1490 | nd | 2830 | 1490 | nd | 2830 | nd | nd | nd |
| Sens 2 | 88% | nd | 83% | 88% | nd | 83% | nd | nd | nd |
| Spec 2 | 62% | nd | 76% | 62% | nd | 76% | nd | nd | nd |
| Cutoff 3 | 0 | nd | 1490 | 0 | nd | 1490 | nd | nd | nd |
| Sens 3 | 100% | nd | 100% | 100% | nd | 100% | nd | nd | nd |
| Spec 3 | 0% | nd | 62% | 0% | nd | 62% | nd | nd | nd |
| Cutoff 4 | 2000 | nd | 2060 | 2000 | nd | 2060 | nd | nd | nd |
| Sens 4 | 75% | nd | 83% | 75% | nd | 83% | nd | nd | nd |
| Spec 4 | 70% | nd | 70% | 70% | nd | 70% | nd | nd | nd |
| Cutoff 5 | 3180 | nd | 3180 | 3180 | nd | 3180 | nd | nd | nd |
| Sens 5 | 62% | nd | 67% | 62% | nd | 67% | nd | nd | nd |
| Spec 5 | 80% | nd | 80% | 80% | nd | 80% | nd | nd | nd |
| Cutoff 6 | 5900 | nd | 5900 | 5900 | nd | 5900 | nd | nd | nd |
| Sens 6 | 38% | nd | 50% | 38% | nd | 50% | nd | nd | nd |
| Spec 6 | 91% | nd | 90% | 91% | nd | 90% | nd | nd | nd |
| OR Quart 2 | 0 | nd | >0 | 0 | nd | >0 | nd | nd | nd |
| p Value | na | nd | <na | na | nd | <na | nd | nd | nd |
| 95% CI of | na | nd | >na | na | nd | >na | nd | nd | nd |
| OR Quart 2 | na | nd | na | na | nd | na | nd | nd | nd |
| OR Quart 3 | 2.0 | nd | >2.2 | 2.0 | nd | >2.2 | nd | nd | nd |
| p Value | 0.58 | nd | <0.53 | 0.58 | nd | <0.53 | nd | nd | nd |
| 95% CI of | 0.17 | nd | >0.19 | 0.17 | nd | >0.19 | nd | nd | nd |
| OR Quart 3 | 24 | nd | na | 24 | nd | na | nd | nd | nd |
| OR Quart 4 | 5.8 | nd | >4.7 | 5.8 | nd | >4.7 | nd | nd | nd |
| p Value | 0.12 | nd | <0.19 | 0.12 | nd | <0.19 | nd | nd | nd |
| 95% CI of | 0.62 | nd | >0.48 | 0.62 | nd | >0.48 | nd | nd | nd |
| OR Quart 4 | 54 | nd | na | 54 | nd | na | nd | nd | nd |

Protransforming growth factor alpha

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 2.51 | 10.2 | 2.51 | 10.2 | 2.51 | 8.34 |
| Average | 8.67 | 30.9 | 8.67 | 30.9 | 8.67 | 16.5 |
| Stdev | 35.8 | 50.3 | 35.8 | 50.4 | 35.8 | 23.6 |
| p (t-test) | | 0.068 | | 0.069 | | 0.57 |
| Min | 0.00305 | 0.566 | 0.00305 | 0.227 | 0.00305 | 0.783 |
| Max | 287 | 173 | 287 | 173 | 287 | 68.3 |
| n (Samp) | 65 | 12 | 65 | 12 | 65 | 7 |
| n (Patient) | 65 | 12 | 65 | 12 | 65 | 7 |
| sCr only | | | | | | |
| Median | 2.57 | 4.38 | 2.57 | 4.38 | nd | nd |
| Average | 9.04 | 14.3 | 9.04 | 14.2 | nd | nd |
| Stdev | 30.5 | 24.0 | 30.5 | 24.1 | nd | nd |
| p (t-test) | | 0.68 | | 0.68 | nd | nd |
| Min | 0.00305 | 0.566 | 0.00305 | 0.227 | nd | nd |
| Max | 287 | 62.6 | 287 | 62.6 | nd | nd |
| n (Samp) | 132 | 6 | 132 | 6 | nd | nd |
| n (Patient) | 132 | 6 | 132 | 6 | nd | nd |
| UO only | | | | | | |
| Median | 2.34 | 12.3 | 2.34 | 12.3 | 2.34 | 10.2 |
| Average | 8.46 | 38.0 | 8.46 | 38.0 | 8.46 | 19.0 |
| Stdev | 36.1 | 58.4 | 36.1 | 58.4 | 36.1 | 24.9 |
| p (t-test) | | 0.047 | | 0.047 | | 0.49 |
| Min | 0.00305 | 0.783 | 0.00305 | 0.783 | 0.00305 | 0.783 |
| Max | 287 | 173 | 287 | 173 | 287 | 68.3 |
| n (Samp) | 64 | 8 | 64 | 8 | 64 | 6 |
| n (Patient) | 64 | 8 | 64 | 8 | 64 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.74 | 0.63 | 0.85 | 0.73 | 0.62 | 0.85 | 0.75 | nd | 0.81 |
| SE | 0.087 | 0.12 | 0.088 | 0.088 | 0.12 | 0.088 | 0.11 | nd | 0.11 |
| p | 0.0060 | 0.28 | 6.8E−5 | 0.0088 | 0.34 | 6.8E−5 | 0.026 | nd | 0.0042 |
| nCohort 1 | 65 | 132 | 64 | 65 | 132 | 64 | 65 | nd | 64 |
| nCohort 2 | 12 | 6 | 8 | 12 | 6 | 8 | 7 | nd | 6 |
| Cutoff 1 | 2.57 | 1.80 | 7.01 | 2.57 | 1.80 | 7.01 | 5.39 | nd | 5.39 |
| Sens 1 | 75% | 83% | 75% | 75% | 83% | 75% | 71% | nd | 83% |
| Spec 1 | 52% | 42% | 89% | 52% | 42% | 89% | 86% | nd | 88% |
| Cutoff 2 | 1.80 | 1.80 | 5.39 | 1.80 | 1.80 | 5.39 | 1.80 | nd | 5.39 |
| Sens 2 | 83% | 83% | 88% | 83% | 83% | 88% | 86% | nd | 83% |
| Spec 2 | 40% | 42% | 88% | 40% | 42% | 88% | 40% | nd | 88% |
| Cutoff 3 | 0.775 | 0.540 | 0.775 | 0.775 | 0.137 | 0.775 | 0.775 | nd | 0.775 |
| Sens 3 | 92% | 100% | 100% | 92% | 100% | 100% | 100% | nd | 100% |
| Spec 3 | 25% | 20% | 30% | 25% | 11% | 30% | 25% | nd | 30% |
| Cutoff 4 | 3.59 | 4.18 | 3.36 | 3.59 | 4.18 | 3.36 | 3.59 | nd | 3.36 |
| Sens 4 | 67% | 50% | 88% | 67% | 50% | 88% | 71% | nd | 83% |
| Spec 4 | 71% | 70% | 70% | 71% | 70% | 70% | 71% | nd | 70% |
| Cutoff 5 | 4.80 | 5.28 | 4.36 | 4.80 | 5.28 | 4.36 | 4.80 | nd | 4.36 |
| Sens 5 | 67% | 50% | 88% | 67% | 50% | 88% | 71% | nd | 83% |
| Spec 5 | 80% | 80% | 81% | 80% | 80% | 81% | 80% | nd | 81% |
| Cutoff 6 | 7.33 | 13.7 | 9.25 | 7.33 | 13.7 | 9.25 | 7.33 | nd | 9.25 |
| Sens 6 | 58% | 17% | 62% | 58% | 17% | 62% | 57% | nd | 50% |
| Spec 6 | 91% | 90% | 91% | 91% | 90% | 91% | 91% | nd | 91% |
| OR Quart 2 | 1.0 | 2.0 | >1.1 | 1.0 | 2.0 | >1.1 | 1.0 | nd | >1.0 |
| p Value | 1.0 | 0.58 | <0.97 | 1.0 | 0.58 | <0.97 | 1.0 | nd | <1.0 |
| 95% CI of | 0.13 | 0.17 | >0.061 | 0.13 | 0.17 | >0.061 | 0.058 | nd | >0.058 |
| OR Quart 2 | 7.9 | 23 | na | 7.9 | 23 | na | 17 | nd | na |
| OR Quart 3 | 0 | 0 | >0 | 0 | 0 | >0 | 0 | nd | >0 |
| p Value | na | na | <na | na | na | <na | na | nd | <na |
| 95% CI of | na | na | >na | na | na | >na | na | nd | >na |
| OR Quart 3 | na | na | na | na | na | na | na | nd | na |
| OR Quart 4 | 5.7 | 3.1 | >11 | 5.7 | 3.1 | >11 | 6.5 | nd | >6.5 |
| p Value | 0.048 | 0.34 | <0.032 | 0.048 | 0.34 | <0.032 | 0.10 | nd | <0.10 |
| 95% CI of | 1.0 | 0.31 | >1.2 | 1.0 | 0.31 | >1.2 | 0.68 | nd | >0.68 |
| OR Quart 4 | 32 | 31 | na | 32 | 31 | na | 63 | nd | na |

CA 15-3

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.245 | 0.833 | 0.245 | 0.644 | nd | nd |
| Average | 0.500 | 1.04 | 0.500 | 0.953 | nd | nd |
| Stdev | 0.916 | 0.811 | 0.916 | 0.817 | nd | nd |
| p (t-test) | | 0.11 | | 0.18 | nd | nd |
| Min | 0.0328 | 0.249 | 0.0328 | 0.249 | nd | nd |
| Max | 6.54 | 2.69 | 6.54 | 2.69 | nd | nd |
| n (Samp) | 87 | 8 | 87 | 8 | nd | nd |
| n (Patient) | 87 | 8 | 87 | 8 | nd | nd |
| UO only | | | | | | |
| Median | 0.289 | 0.588 | 0.289 | 0.588 | nd | nd |
| Average | 0.616 | 1.01 | 0.616 | 1.01 | nd | nd |
| Stdev | 1.07 | 0.952 | 1.07 | 0.952 | nd | nd |
| p (t-test) | | 0.39 | | 0.39 | nd | nd |
| Min | 0.0328 | 0.249 | 0.0328 | 0.249 | nd | nd |
| Max | 6.54 | 2.69 | 6.54 | 2.69 | nd | nd |
| n (Samp) | 80 | 6 | 80 | 6 | nd | nd |
| n (Patient) | 80 | 6 | 80 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.82 | nd | 0.75 | 0.81 | nd | 0.75 | nd | nd | nd |
| SE | 0.093 | nd | 0.12 | 0.094 | nd | 0.12 | nd | nd | nd |
| p | 5.7E−4 | nd | 0.039 | 8.9E−4 | nd | 0.039 | nd | nd | nd |
| nCohort 1 | 87 | nd | 80 | 87 | nd | 80 | nd | nd | nd |
| nCohort 2 | 8 | nd | 6 | 8 | nd | 6 | nd | nd | nd |
| Cutoff 1 | 0.476 | nd | 0.346 | 0.476 | nd | 0.346 | nd | nd | nd |
| Sens 1 | 75% | nd | 83% | 75% | nd | 83% | nd | nd | nd |
| Spec 1 | 83% | nd | 59% | 83% | nd | 59% | nd | nd | nd |
| Cutoff 2 | 0.326 | nd | 0.346 | 0.326 | nd | 0.346 | nd | nd | nd |
| Sens 2 | 88% | nd | 83% | 88% | nd | 83% | nd | nd | nd |
| Spec 2 | 68% | nd | 59% | 68% | nd | 59% | nd | nd | nd |
| Cutoff 3 | 0.248 | nd | 0.248 | 0.248 | nd | 0.248 | nd | nd | nd |
| Sens 3 | 100% | nd | 100% | 100% | nd | 100% | nd | nd | nd |
| Spec 3 | 54% | nd | 46% | 54% | nd | 46% | nd | nd | nd |
| Cutoff 4 | 0.353 | nd | 0.402 | 0.353 | nd | 0.402 | nd | nd | nd |
| Sens 4 | 75% | nd | 67% | 75% | nd | 67% | nd | nd | nd |
| Spec 4 | 70% | nd | 70% | 70% | nd | 70% | nd | nd | nd |
| Cutoff 5 | 0.466 | nd | 0.708 | 0.466 | nd | 0.708 | nd | nd | nd |
| Sens 5 | 75% | nd | 33% | 75% | nd | 33% | nd | nd | nd |
| Spec 5 | 80% | nd | 80% | 80% | nd | 80% | nd | nd | nd |
| Cutoff 6 | 1.28 | nd | 1.57 | 1.28 | nd | 1.57 | nd | nd | nd |
| Sens 6 | 25% | nd | 33% | 25% | nd | 33% | nd | nd | nd |
| Spec 6 | 91% | nd | 90% | 91% | nd | 90% | nd | nd | nd |
| OR Quart 2 | >0 | nd | >1.0 | >0 | nd | >1.0 | nd | nd | nd |
| p Value | <na | nd | <1.0 | <na | nd | <1.0 | nd | nd | nd |
| 95% CI of | >na | nd | >0.059 | >na | nd | >0.059 | nd | nd | nd |
| OR Quart 2 | na | nd | na | na | nd | na | nd | nd | nd |
| OR Quart 3 | >2.1 | nd | >1.0 | >2.1 | nd | >1.0 | nd | nd | nd |
| p Value | <0.56 | nd | <0.97 | <0.56 | nd | <0.97 | nd | nd | nd |
| 95% CI of | >0.18 | nd | >0.061 | >0.18 | nd | >0.061 | nd | nd | nd |
| OR Quart 3 | na | nd | na | na | nd | na | nd | nd | nd |
| OR Quart 4 | >7.7 | nd | >4.7 | >7.7 | nd | >4.7 | nd | nd | nd |
| p Value | <0.070 | nd | <0.19 | <0.070 | nd | <0.19 | nd | nd | nd |
| 95% CI of | >0.85 | nd | >0.48 | >0.85 | nd | >0.48 | nd | nd | nd |
| OR Quart 4 | na | nd | na | na | nd | na | nd | nd | nd |

TABLE 9
Comparison of marker levels in urine samples collected from Cohort 1
(patients that did not progress beyond RIFLE stage 0, R, or I) and in urine samples
collected from Cohort 2 (subjects who progress to RIFLE stage F) at 0, 24 hours, and 48
hours prior to the subject reaching RIFLE stage I.
C-C motif chemokine 18

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.474 | 2.53 | 0.474 | 3.21 | 0.474 | 0.628 |
| Average | 2.27 | 6.02 | 2.27 | 10.9 | 2.27 | 12.6 |
| Stdev | 6.63 | 9.81 | 6.63 | 14.5 | 6.63 | 18.9 |
| p (t-test) | | 0.0092 | | 2.1E−8 | | 1.7E−6 |
| Min | 3.13E−5 | 0.0680 | 3.13E−5 | 0.138 | 3.13E−5 | 0.00268 |
| Max | 40.0 | 40.0 | 40.0 | 40.0 | 40.0 | 40.0 |
| n (Samp) | 1273 | 22 | 1273 | 20 | 1273 | 10 |
| n (Patient) | 451 | 22 | 451 | 20 | 451 | 10 |
| sCr only | | | | | | |
| Median | 0.490 | 4.14 | nd | nd | nd | nd |
| Average | 2.63 | 9.65 | nd | nd | nd | nd |
| Stdev | 7.46 | 10.8 | nd | nd | nd | nd |
| p (t-test) | | 0.0082 | nd | nd | nd | nd |
| Min | 3.13E−5 | 0.204 | nd | nd | nd | nd |
| Max | 40.0 | 31.3 | nd | nd | nd | nd |
| n (Samp) | 1337 | 8 | nd | nd | nd | nd |
| n (Patient) | 466 | 8 | nd | nd | nd | nd |
| UO only | | | | | | |
| Median | 0.490 | 2.16 | 0.490 | 3.58 | 0.490 | 0.724 |
| Average | 2.46 | 8.94 | 2.46 | 13.1 | 2.46 | 17.3 |
| Stdev | 6.94 | 14.1 | 6.94 | 16.2 | 6.94 | 21.2 |
| p (t-test) | | 6.6E−4 | | 2.3E−10 | | 4.2E−8 |
| Min | 3.13E−5 | 0.0680 | 3.13E−5 | 0.138 | 3.13E−5 | 0.119 |
| Max | 40.0 | 40.0 | 40.0 | 40.0 | 40.0 | 40.0 |
| n (Samp) | 1119 | 14 | 1119 | 19 | 1119 | 7 |
| n (Patient) | 361 | 14 | 361 | 19 | 361 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.72 | 0.83 | 0.71 | 0.78 | nd | 0.79 | 0.56 | nd | 0.58 |
| SE | 0.062 | 0.090 | 0.079 | 0.062 | nd | 0.062 | 0.094 | nd | 0.11 |
| p | 4.1E−4 | 2.4E−4 | 0.0090 | 7.2E−6 | nd | 2.8E−6 | 0.53 | nd | 0.48 |
| nCohort 1 | 1273 | 1337 | 1119 | 1273 | nd | 1119 | 1273 | nd | 1119 |
| nCohort 2 | 22 | 8 | 14 | 20 | nd | 19 | 10 | nd | 7 |
| Cutoff 1 | 0.862 | 2.72 | 0.862 | 1.98 | nd | 1.98 | 0.131 | nd | 0.131 |
| Sens 1 | 73% | 75% | 71% | 70% | nd | 74% | 70% | nd | 71% |
| Spec 1 | 65% | 85% | 63% | 83% | nd | 81% | 19% | nd | 17% |
| Cutoff 2 | 0.351 | 2.33 | 0.232 | 0.788 | nd | 0.788 | 0.125 | nd | 0.125 |
| Sens 2 | 82% | 88% | 86% | 80% | nd | 84% | 80% | nd | 86% |
| Spec 2 | 43% | 83% | 29% | 63% | nd | 62% | 18% | nd | 17% |
| Cutoff 3 | 0.204 | 0.204 | 0.164 | 0.270 | nd | 0.142 | 0.118 | nd | 0.118 |
| Sens 3 | 91% | 100% | 93% | 90% | nd | 95% | 90% | nd | 100% |
| Spec 3 | 27% | 27% | 22% | 36% | nd | 19% | 18% | nd | 16% |
| Cutoff 4 | 1.04 | 1.10 | 1.12 | 1.04 | nd | 1.12 | 1.04 | nd | 1.12 |
| Sens 4 | 68% | 88% | 64% | 75% | nd | 79% | 40% | nd | 43% |
| Spec 4 | 70% | 70% | 70% | 70% | nd | 70% | 70% | nd | 70% |
| Cutoff 5 | 1.76 | 1.90 | 1.90 | 1.76 | nd | 1.90 | 1.76 | nd | 1.90 |
| Sens 5 | 55% | 88% | 50% | 70% | nd | 74% | 40% | nd | 43% |
| Spec 5 | 80% | 80% | 80% | 80% | nd | 80% | 80% | nd | 80% |
| Cutoff 6 | 3.72 | 4.03 | 3.96 | 3.72 | nd | 3.96 | 3.72 | nd | 3.96 |
| Sens 6 | 36% | 50% | 43% | 45% | nd | 47% | 40% | nd | 43% |
| Spec 6 | 90% | 90% | 90% | 90% | nd | 90% | 90% | nd | 90% |
| OR Quart 2 | 1.5 | >1.0 | 0.50 | 1.0 | nd | 0.50 | 0 | nd | 0 |
| p Value | 0.66 | <1.00 | 0.57 | 1.0 | nd | 0.57 | na | nd | na |
| 95% CI of | 0.25 | >0.062 | 0.045 | 0.14 | nd | 0.045 | na | nd | na |
| OR Quart 2 | 9.0 | na | 5.5 | 7.1 | nd | 5.5 | na | nd | na |
| OR Quart 3 | 2.0 | >0 | 1.5 | 0.50 | nd | 0.50 | 0.50 | nd | 0.33 |
| p Value | 0.42 | <na | 0.66 | 0.57 | nd | 0.57 | 0.42 | nd | 0.34 |
| 95% CI of | 0.36 | >na | 0.25 | 0.045 | nd | 0.045 | 0.090 | nd | 0.034 |
| OR Quart 3 | 11 | na | 9.1 | 5.5 | nd | 5.5 | 2.7 | nd | 3.2 |
| OR Quart 4 | 6.7 | >7.1 | 4.1 | 7.8 | nd | 7.8 | 1.00 | nd | 1.00 |
| p Value | 0.013 | <0.067 | 0.077 | 0.0067 | nd | 0.0066 | 1.00 | nd | 1.00 |
| 95% CI of | 1.5 | >0.87 | 0.86 | 1.8 | nd | 1.8 | 0.25 | nd | 0.20 |
| OR Quart 4 | 30 | na | 19 | 34 | nd | 35 | 4.0 | nd | 5.0 |

C-C motif chemokine 24

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 12.9 | 39.0 | 12.9 | 34.2 | 12.9 | 14.0 |
| Average | 28.6 | 216 | 28.6 | 269 | 28.6 | 127 |
| Stdev | 58.5 | 474 | 58.5 | 594 | 58.5 | 368 |
| p (t-test) | | 1.8E−24 | | 2.4E−29 | | 2.6E−6 |
| Min | 0.0120 | 5.58 | 0.0120 | 0.0347 | 0.0120 | 0.0635 |
| Max | 1090 | 1930 | 1090 | 2380 | 1090 | 1170 |
| n (Samp) | 1275 | 22 | 1275 | 20 | 1275 | 10 |
| n (Patient) | 452 | 22 | 452 | 20 | 452 | 10 |
| sCr only | | | | | | |
| Median | 13.7 | 57.3 | nd | nd | nd | nd |
| Average | 42.1 | 88.9 | nd | nd | nd | nd |
| Stdev | 152 | 118 | nd | nd | nd | nd |
| p (t-test) | | 0.38 | nd | nd | nd | nd |
| Min | 0.0120 | 11.1 | nd | nd | nd | nd |
| Max | 2790 | 373 | nd | nd | nd | nd |
| n (Samp) | 1339 | 8 | nd | nd | nd | nd |
| n (Patient) | 467 | 8 | nd | nd | nd | nd |
| UO only | | | | | | |
| Median | 13.2 | 56.4 | 13.2 | 37.2 | 13.2 | 14.8 |
| Average | 29.6 | 391 | 29.6 | 384 | 29.6 | 178 |
| Stdev | 60.6 | 769 | 60.6 | 710 | 60.6 | 439 |
| p (t-test) | | 6.3E−37 | | 1.8E−42 | | 1.3E−8 |
| Min | 0.0120 | 5.58 | 0.0120 | 0.0347 | 0.0120 | 4.32 |
| Max | 1090 | 2790 | 1090 | 2380 | 1090 | 1170 |
| n (Samp) | 1122 | 14 | 1122 | 19 | 1122 | 7 |
| n (Patient) | 362 | 14 | 362 | 19 | 362 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.73 | 0.79 | 0.76 | 0.71 | nd | 0.76 | 0.50 | nd | 0.53 |
| SE | 0.062 | 0.096 | 0.075 | 0.066 | nd | 0.065 | 0.092 | nd | 0.11 |
| p | 1.7E−4 | 0.0031 | 6.2E−4 | 0.0012 | nd | 6.7E−5 | 1.00 | nd | 0.77 |
| nCohort 1 | 1275 | 1339 | 1122 | 1275 | nd | 1122 | 1275 | nd | 1122 |
| nCohort 2 | 22 | 8 | 14 | 20 | nd | 19 | 10 | nd | 7 |
| Cutoff 1 | 21.9 | 25.9 | 22.5 | 27.0 | nd | 27.2 | 6.94 | nd | 6.94 |
| Sens 1 | 73% | 75% | 71% | 70% | nd | 74% | 70% | nd | 71% |
| Spec 1 | 65% | 68% | 66% | 71% | nd | 71% | 37% | nd | 36% |
| Cutoff 2 | 9.98 | 21.9 | 8.63 | 14.2 | nd | 16.6 | 4.59 | nd | 4.59 |
| Sens 2 | 82% | 88% | 86% | 80% | nd | 84% | 80% | nd | 86% |
| Spec 2 | 44% | 63% | 40% | 53% | nd | 57% | 30% | nd | 29% |
| Cutoff 3 | 8.65 | 11.1 | 5.84 | 3.78 | nd | 3.78 | 4.24 | nd | 4.24 |
| Sens 3 | 91% | 100% | 93% | 90% | nd | 95% | 90% | nd | 100% |
| Spec 3 | 42% | 45% | 32% | 29% | nd | 28% | 30% | nd | 28% |
| Cutoff 4 | 26.4 | 28.1 | 26.7 | 26.4 | nd | 26.7 | 26.4 | nd | 26.7 |
| Sens 4 | 59% | 62% | 64% | 70% | nd | 79% | 10% | nd | 14% |
| Spec 4 | 70% | 70% | 70% | 70% | nd | 70% | 70% | nd | 70% |
| Cutoff 5 | 40.3 | 42.2 | 40.9 | 40.3 | nd | 40.9 | 40.3 | nd | 40.9 |
| Sens 5 | 50% | 62% | 64% | 40% | nd | 47% | 10% | nd | 14% |
| Spec 5 | 80% | 80% | 80% | 80% | nd | 80% | 80% | nd | 80% |
| Cutoff 6 | 69.6 | 78.2 | 72.3 | 69.6 | nd | 72.3 | 69.6 | nd | 72.3 |
| Sens 6 | 32% | 38% | 43% | 35% | nd | 37% | 10% | nd | 14% |
| Spec 6 | 90% | 90% | 90% | 90% | nd | 90% | 90% | nd | 90% |
| OR Quart 2 | >6.1 | >1.0 | >4.1 | 1.00 | nd | 2.0 | 3.0 | nd | >3.0 |
| p Value | <0.095 | <1.0 | <0.21 | 1.00 | nd | 0.57 | 0.34 | nd | <0.34 |
| 95% CI of | >0.73 | >0.062 | >0.45 | 0.14 | nd | 0.18 | 0.31 | nd | >0.31 |
| OR Quart 2 | na | na | na | 7.1 | nd | 22 | 29 | nd | na |
| OR Quart 3 | >5.1 | >2.0 | >1.0 | 3.0 | nd | 5.1 | 5.1 | nd | >3.0 |
| p Value | <0.14 | <0.57 | <1.00 | 0.18 | nd | 0.14 | 0.14 | nd | <0.34 |
| 95% CI of | >0.59 | >0.18 | >0.062 | 0.61 | nd | 0.59 | 0.59 | nd | >0.31 |
| OR Quart 3 | na | na | na | 15 | nd | 44 | 44 | nd | na |
| OR Quart 4 | >11 | >5.1 | >9.3 | 5.1 | nd | 11 | 1.00 | nd | >1.0 |
| p Value | <0.020 | <0.14 | <0.035 | 0.036 | nd | 0.020 | 1.00 | nd | <1.0 |
| 95% CI of | >1.5 | >0.59 | >1.2 | 1.1 | nd | 1.5 | 0.062 | nd | >0.062 |
| OR Quart 4 | na | na | na | 24 | nd | 89 | 16 | nd | na |

C-C motif chemokine 8

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.36 | 23.8 | 1.36 | 10.0 | 1.36 | 1.61 |
| Average | 9.75 | 211 | 9.75 | 42.0 | 9.75 | 16.5 |
| Stdev | 26.6 | 775 | 26.6 | 70.5 | 26.6 | 34.1 |
| p (t-test) | | 1.7E−19 | | 2.9E−7 | | 0.43 |
| Min | 0.0250 | 0.206 | 0.0250 | 0.0945 | 0.0250 | 0.0472 |
| Max | 473 | 3670 | 473 | 294 | 473 | 111 |
| n (Samp) | 1275 | 22 | 1275 | 20 | 1275 | 10 |
| n (Patient) | 452 | 22 | 452 | 20 | 452 | 10 |
| sCr only | | | | | | |
| Median | 1.42 | 16.3 | nd | nd | nd | nd |
| Average | 14.1 | 29.6 | nd | nd | nd | nd |
| Stdev | 105 | 40.1 | nd | nd | nd | nd |
| p (t-test) | | 0.68 | nd | nd | nd | nd |
| Min | 0.0250 | 0.206 | nd | nd | nd | nd |
| Max | 3670 | 116 | nd | nd | nd | nd |
| n (Samp) | 1339 | 8 | nd | nd | nd | nd |
| n (Patient) | 467 | 8 | nd | nd | nd | nd |
| UO only | | | | | | |
| Median | 1.36 | 31.3 | 1.36 | 30.8 | 1.36 | 2.77 |
| Average | 10.1 | 59.3 | 10.1 | 51.5 | 10.1 | 20.2 |
| Stdev | 27.7 | 66.2 | 27.7 | 71.6 | 27.7 | 40.5 |
| p (t-test) | | 2.0E−10 | | 9.3E−10 | | 0.34 |
| Min | 0.0250 | 0.829 | 0.0250 | 0.136 | 0.0250 | 0.0472 |
| Max | 473 | 176 | 473 | 294 | 473 | 111 |
| n (Samp) | 1122 | 14 | 1122 | 19 | 1122 | 7 |
| n (Patient) | 362 | 14 | 362 | 19 | 362 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.77 | 0.71 | 0.81 | 0.73 | nd | 0.79 | 0.52 | nd | 0.55 |
| SE | 0.059 | 0.10 | 0.071 | 0.065 | nd | 0.062 | 0.093 | nd | 0.11 |
| p | 3.7E−6 | 0.046 | 1.4E−5 | 4.2E−4 | nd | 3.7E−6 | 0.79 | nd | 0.68 |
| nCohort 1 | 1275 | 1339 | 1122 | 1275 | nd | 1122 | 1275 | nd | 1122 |
| nCohort 2 | 22 | 8 | 14 | 20 | nd | 19 | 10 | nd | 7 |
| Cutoff 1 | 2.74 | 2.74 | 12.6 | 3.20 | nd | 8.37 | 0.240 | nd | 0.438 |
| Sens 1 | 77% | 75% | 71% | 70% | nd | 74% | 70% | nd | 71% |
| Spec 1 | 56% | 55% | 82% | 60% | nd | 74% | 34% | nd | 39% |
| Cutoff 2 | 0.802 | 0.491 | 0.802 | 1.21 | nd | 1.21 | 0.127 | nd | 0.127 |
| Sens 2 | 91% | 88% | 100% | 80% | nd | 84% | 80% | nd | 86% |
| Spec 2 | 44% | 38% | 44% | 49% | nd | 49% | 21% | nd | 20% |
| Cutoff 3 | 0.802 | 0.154 | 0.802 | 0.491 | nd | 0.491 | 0.0705 | nd | 0.0250 |
| Sens 3 | 91% | 100% | 100% | 90% | nd | 95% | 90% | nd | 100% |
| Spec 3 | 44% | 26% | 44% | 40% | nd | 39% | 11% | nd | 2% |
| Cutoff 4 | 6.78 | 7.38 | 6.78 | 6.78 | nd | 6.78 | 6.78 | nd | 6.78 |
| Sens 4 | 68% | 62% | 71% | 65% | nd | 74% | 40% | nd | 43% |
| Spec 4 | 70% | 70% | 70% | 70% | nd | 70% | 70% | nd | 70% |
| Cutoff 5 | 11.5 | 12.5 | 11.8 | 11.5 | nd | 11.8 | 11.5 | nd | 11.8 |
| Sens 5 | 64% | 50% | 71% | 45% | nd | 58% | 40% | nd | 29% |
| Spec 5 | 80% | 80% | 80% | 80% | nd | 80% | 80% | nd | 80% |
| Cutoff 6 | 22.8 | 27.7 | 23.0 | 22.8 | nd | 23.0 | 22.8 | nd | 23.0 |
| Sens 6 | 50% | 25% | 57% | 45% | nd | 58% | 10% | nd | 14% |
| Spec 6 | 90% | 90% | 90% | 90% | nd | 90% | 90% | nd | 90% |
| OR Quart 2 | >5.1 | >2.0 | >3.0 | 1.00 | nd | 2.0 | 0.66 | nd | 0.50 |
| p Value | <0.14 | <0.57 | <0.34 | 1.00 | nd | 0.57 | 0.66 | nd | 0.57 |
| 95% CI of | >0.59 | >0.18 | >0.31 | 0.14 | nd | 0.18 | 0.11 | nd | 0.045 |
| OR Quart 2 | na | na | na | 7.1 | nd | 22 | 4.0 | nd | 5.5 |
| OR Quart 3 | >3.0 | >2.0 | >1.0 | 2.5 | nd | 3.0 | 0.33 | nd | 0.50 |
| p Value | <0.34 | <0.57 | <1.00 | 0.27 | nd | 0.34 | 0.34 | nd | 0.57 |
| 95% CI of | >0.31 | >0.18 | >0.062 | 0.48 | nd | 0.31 | 0.034 | nd | 0.045 |
| OR Quart 3 | na | na | na | 13 | nd | 29 | 3.2 | nd | 5.5 |
| OR Quart 4 | >15 | >4.0 | >10 | 5.6 | nd | 14 | 1.3 | nd | 1.5 |
| p Value | <0.0098 | <0.21 | <0.026 | 0.025 | nd | 0.012 | 0.71 | nd | 0.66 |
| 95% CI of | >1.9 | >0.45 | >1.3 | 1.2 | nd | 1.8 | 0.30 | nd | 0.25 |
| OR Quart 4 | na | na | na | 26 | nd | 100 | 6.0 | nd | 9.0 |

Cathepsin D

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 75900 | 115000 | 75900 | 114000 | 75900 | 101000 |
| Average | 81800 | 121000 | 81800 | 117000 | 81800 | 95500 |
| Stdev | 41500 | 43500 | 41500 | 43600 | 41500 | 52800 |
| p (t-test) | | 1.2E−5 | | 1.4E−4 | | 0.30 |
| Min | 656 | 63100 | 656 | 45000 | 656 | 4320 |
| Max | 200000 | 200000 | 200000 | 200000 | 200000 | 200000 |
| n (Samp) | 1273 | 22 | 1273 | 20 | 1273 | 10 |
| n (Patient) | 451 | 22 | 451 | 20 | 451 | 10 |
| sCr only | | | | | | |
| Median | 78100 | 99300 | nd | nd | nd | nd |
| Average | 84000 | 109000 | nd | nd | nd | nd |
| Stdev | 43000 | 32700 | nd | nd | nd | nd |
| p (t-test) | | 0.10 | nd | nd | nd | nd |
| Min | 656 | 71400 | nd | nd | nd | nd |
| Max | 200000 | 151000 | nd | nd | nd | nd |
| n (Samp) | 1337 | 8 | nd | nd | nd | nd |
| n (Patient) | 466 | 8 | nd | nd | nd | nd |
| UO only | | | | | | |
| Median | 78500 | 132000 | 78500 | 117000 | 78500 | 99800 |
| Average | 84100 | 128000 | 84100 | 123000 | 84100 | 104000 |
| Stdev | 41200 | 47600 | 41200 | 46300 | 41200 | 51000 |
| p (t-test) | | 7.8E−5 | | 4.2E−5 | | 0.21 |
| Min | 2520 | 63100 | 2520 | 45000 | 2520 | 54900 |
| Max | 200000 | 200000 | 200000 | 200000 | 200000 | 200000 |
| n (Samp) | 1119 | 14 | 1119 | 19 | 1119 | 7 |
| n (Patient) | 361 | 14 | 361 | 19 | 361 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.75 | 0.69 | 0.76 | 0.73 | nd | 0.74 | 0.59 | nd | 0.61 |
| SE | 0.061 | 0.10 | 0.075 | 0.065 | nd | 0.066 | 0.095 | nd | 0.11 |
| p | 5.9E−5 | 0.063 | 6.5E−4 | 3.8E−4 | nd | 2.7E−4 | 0.34 | nd | 0.36 |
| nCohort 1 | 1273 | 1337 | 1119 | 1273 | nd | 1119 | 1273 | nd | 1119 |
| nCohort 2 | 22 | 8 | 14 | 20 | nd | 19 | 10 | nd | 7 |
| Cutoff 1 | 84700 | 89600 | 89600 | 90800 | nd | 90800 | 66900 | nd | 66900 |
| Sens 1 | 73% | 75% | 71% | 70% | nd | 74% | 70% | nd | 71% |
| Spec 1 | 58% | 60% | 61% | 64% | nd | 62% | 40% | nd | 37% |
| Cutoff 2 | 76300 | 73800 | 76300 | 84400 | nd | 84400 | 63400 | nd | 63400 |
| Sens 2 | 82% | 88% | 86% | 80% | nd | 84% | 80% | nd | 86% |
| Spec 2 | 50% | 46% | 48% | 58% | nd | 56% | 36% | nd | 33% |
| Cutoff 3 | 71400 | 71400 | 70900 | 73700 | nd | 65500 | 54900 | nd | 54900 |
| Sens 3 | 91% | 100% | 93% | 90% | nd | 95% | 90% | nd | 100% |
| Spec 3 | 45% | 43% | 42% | 47% | nd | 35% | 27% | nd | 24% |
| Cutoff 4 | 101000 | 104000 | 103000 | 101000 | nd | 103000 | 101000 | nd | 103000 |
| Sens 4 | 59% | 50% | 64% | 60% | nd | 63% | 50% | nd | 43% |
| Spec 4 | 70% | 70% | 70% | 70% | nd | 70% | 70% | nd | 70% |
| Cutoff 5 | 116000 | 119000 | 118000 | 116000 | nd | 118000 | 116000 | nd | 118000 |
| Sens 5 | 50% | 38% | 57% | 45% | nd | 47% | 30% | nd | 29% |
| Spec 5 | 80% | 80% | 80% | 80% | nd | 80% | 80% | nd | 80% |
| Cutoff 6 | 137000 | 144000 | 140000 | 137000 | nd | 140000 | 137000 | nd | 140000 |
| Sens 6 | 41% | 25% | 50% | 25% | nd | 26% | 10% | nd | 14% |
| Spec 6 | 90% | 90% | 90% | 90% | nd | 90% | 90% | nd | 90% |
| OR Quart 2 | >5.1 | >2.0 | >3.0 | 2.0 | nd | 2.0 | 3.0 | nd | 2.0 |
| p Value | <0.14 | <0.57 | <0.34 | 0.57 | nd | 0.57 | 0.34 | nd | 0.57 |
| 95% CI of | >0.59 | >0.18 | >0.31 | 0.18 | nd | 0.18 | 0.31 | nd | 0.18 |
| OR Quart 2 | na | na | na | 22 | nd | 22 | 29 | nd | 22 |
| OR Quart 3 | >4.0 | >3.0 | >3.0 | 5.1 | nd | 5.1 | 3.0 | nd | 2.0 |
| p Value | <0.21 | <0.34 | <0.34 | 0.14 | nd | 0.14 | 0.34 | nd | 0.57 |
| 95% CI of | >0.45 | >0.31 | >0.31 | 0.59 | nd | 0.59 | 0.31 | nd | 0.18 |
| OR Quart 3 | na | na | na | 44 | nd | 44 | 29 | nd | 22 |
| OR Quart 4 | >14 | >3.0 | >8.2 | 12 | nd | 11 | 3.0 | nd | 2.0 |
| p Value | <0.012 | <0.34 | <0.048 | 0.016 | nd | 0.020 | 0.34 | nd | 0.57 |
| 95% CI of | >1.8 | >0.31 | >1.0 | 1.6 | nd | 1.5 | 0.31 | nd | 0.18 |
| OR Quart 4 | na | na | na | 96 | nd | 89 | 29 | nd | 22 |

C—X—C motif chemokine 13

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.0398 | 6.51 | 0.0398 | 13.3 | 0.0398 | 2.67 |
| Average | 6.13 | 110 | 6.13 | 38.0 | 6.13 | 52.4 |
| Stdev | 35.0 | 392 | 35.0 | 47.6 | 35.0 | 138 |
| p (t-test) | | 5.5E−15 | | 6.6E−5 | | 7.7E−5 |
| Min | 0.00269 | 0.0104 | 0.00269 | 0.00471 | 0.00269 | 0.00471 |
| Max | 832 | 1850 | 832 | 131 | 832 | 440 |
| n (Samp) | 1275 | 22 | 1275 | 20 | 1275 | 10 |
| n (Patient) | 452 | 22 | 452 | 20 | 452 | 10 |
| sCr only | | | | | | |
| Median | 0.145 | 3.98 | nd | nd | nd | nd |
| Average | 11.2 | 13.7 | nd | nd | nd | nd |
| Stdev | 83.4 | 27.2 | nd | nd | nd | nd |
| p (t-test) | | 0.93 | nd | nd | nd | nd |
| Min | 0.00269 | 0.0174 | nd | nd | nd | nd |
| Max | 1930 | 80.5 | nd | nd | nd | nd |
| n (Samp) | 1339 | 8 | nd | nd | nd | nd |
| n (Patient) | 467 | 8 | nd | nd | nd | nd |
| UO only | | | | | | |
| Median | 0.116 | 20.2 | 0.116 | 30.0 | 0.116 | 2.90 |
| Average | 6.45 | 169 | 6.45 | 142 | 6.45 | 74.4 |
| Stdev | 37.0 | 510 | 37.0 | 418 | 37.0 | 163 |
| p (t-test) | | 2.2E−19 | | 3.4E−19 | | 4.1E−6 |
| Min | 0.00269 | 0.0104 | 0.00269 | 0.00471 | 0.00269 | 0.00821 |
| Max | 832 | 1930 | 832 | 1850 | 832 | 440 |
| n (Samp) | 1122 | 14 | 1122 | 19 | 1122 | 7 |
| n (Patient) | 362 | 14 | 362 | 19 | 362 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.79 | 0.77 | 0.81 | 0.81 | nd | 0.84 | 0.59 | nd | 0.68 |
| SE | 0.058 | 0.098 | 0.071 | 0.059 | nd | 0.058 | 0.095 | nd | 0.11 |
| p | 8.4E−7 | 0.0051 | 1.5E−5 | 2.0E−7 | nd | 6.1E−9 | 0.33 | nd | 0.12 |
| nCohort 1 | 1275 | 1339 | 1122 | 1275 | nd | 1122 | 1275 | nd | 1122 |
| nCohort 2 | 22 | 8 | 14 | 20 | nd | 19 | 10 | nd | 7 |
| Cutoff 1 | 2.00 | 2.57 | 2.86 | 2.60 | nd | 3.17 | 0.0109 | nd | 2.43 |
| Sens 1 | 73% | 75% | 71% | 70% | nd | 74% | 70% | nd | 71% |
| Spec 1 | 74% | 75% | 78% | 77% | nd | 79% | 26% | nd | 76% |
| Cutoff 2 | 0.362 | 2.00 | 0.362 | 1.19 | nd | 1.19 | 0.00930 | nd | 0.00930 |
| Sens 2 | 82% | 88% | 86% | 80% | nd | 84% | 80% | nd | 86% |
| Spec 2 | 55% | 71% | 54% | 67% | nd | 66% | 25% | nd | 23% |
| Cutoff 3 | 0.0163 | 0.0163 | 0.0155 | 0.0193 | nd | 0.0174 | 0.00789 | nd | 0.00789 |
| Sens 3 | 91% | 100% | 93% | 90% | nd | 95% | 90% | nd | 100% |
| Spec 3 | 45% | 44% | 42% | 47% | nd | 44% | 18% | nd | 16% |
| Cutoff 4 | 1.51 | 1.76 | 1.54 | 1.51 | nd | 1.54 | 1.51 | nd | 1.54 |
| Sens 4 | 73% | 88% | 71% | 75% | nd | 79% | 60% | nd | 71% |
| Spec 4 | 70% | 70% | 70% | 70% | nd | 70% | 70% | nd | 70% |
| Cutoff 5 | 3.37 | 3.89 | 3.44 | 3.37 | nd | 3.44 | 3.37 | nd | 3.44 |
| Sens 5 | 55% | 50% | 64% | 60% | nd | 68% | 30% | nd | 43% |
| Spec 5 | 80% | 80% | 80% | 80% | nd | 80% | 80% | nd | 80% |
| Cutoff 6 | 9.59 | 12.3 | 9.51 | 9.59 | nd | 9.51 | 9.59 | nd | 9.51 |
| Sens 6 | 41% | 12% | 57% | 55% | nd | 63% | 20% | nd | 29% |
| Spec 6 | 90% | 90% | 90% | 90% | nd | 90% | 90% | nd | 90% |
| OR Quart 2 | >4.0 | >1.0 | 1.0 | 2.0 | nd | 1.0 | 1.0 | nd | 0 |
| p Value | <0.21 | <1.0 | 1.0 | 0.57 | nd | 1.0 | 1.0 | nd | na |
| 95% CI of | >0.45 | >0.062 | 0.062 | 0.18 | nd | 0.062 | 0.14 | nd | na |
| OR Quart 2 | na | na | 16 | 22 | nd | 16 | 7.1 | nd | na |
| OR Quart 3 | >3.0 | >2.0 | 2.0 | 3.0 | nd | 3.0 | 0 | nd | 0 |
| p Value | <0.34 | <0.57 | 0.57 | 0.34 | nd | 0.34 | na | nd | na |
| 95% CI of | >0.31 | >0.18 | 0.18 | 0.31 | nd | 0.31 | na | nd | na |
| OR Quart 3 | na | na | 22 | 29 | nd | 29 | na | nd | na |
| OR Quart 4 | >16 | >5.1 | 10 | 15 | nd | 15 | 3.0 | nd | 2.5 |
| p Value | <0.0079 | <0.14 | 0.026 | 0.0099 | nd | 0.0098 | 0.18 | nd | 0.27 |
| 95% CI of | >2.1 | >0.59 | 1.3 | 1.9 | nd | 1.9 | 0.61 | nd | 0.48 |
| OR Quart 4 | na | na | 81 | 110 | nd | 110 | 15 | nd | 13 |

Insulin-like growth factor-binding protein 3

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 392 | 551 | 392 | 670 | 392 | 609 |
| Average | 672 | 1350 | 672 | 1760 | 672 | 684 |
| Stdev | 1010 | 1580 | 1010 | 2850 | 1010 | 546 |
| p (t-test) | | 0.0012 | | 7.6E−7 | | 0.96 |
| Min | 0.0478 | 88.0 | 0.0478 | 98.3 | 0.0478 | 0.311 |
| Max | 12500 | 5660 | 12500 | 12500 | 12500 | 1550 |
| n (Samp) | 1385 | 24 | 1385 | 24 | 1385 | 13 |
| n (Patient) | 484 | 24 | 484 | 24 | 484 | 13 |
| sCr only | | | | | | |
| Median | 410 | 786 | 410 | 1560 | 410 | 862 |
| Average | 730 | 1260 | 730 | 2430 | 730 | 931 |
| Stdev | 1190 | 1440 | 1190 | 2880 | 1190 | 559 |
| p (t-test) | | 0.21 | | 5.4E−4 | | 0.65 |
| Min | 0.0478 | 88.0 | 0.0478 | 455 | 0.0478 | 267 |
| Max | 12500 | 4040 | 12500 | 8160 | 12500 | 1880 |
| n (Samp) | 1452 | 8 | 1452 | 6 | 1452 | 7 |
| n (Patient) | 500 | 8 | 500 | 6 | 500 | 7 |
| UO only | | | | | | |
| Median | 419 | 993 | 419 | 947 | 419 | 609 |
| Average | 701 | 2620 | 701 | 2080 | 701 | 753 |
| Stdev | 1080 | 3340 | 1080 | 3020 | 1080 | 673 |
| p (t-test) | | 1.0E−10 | | 4.5E−8 | | 0.90 |
| Min | 0.0478 | 119 | 0.0478 | 98.3 | 0.0478 | 65.9 |
| Max | 12500 | 12500 | 12500 | 12500 | 12500 | 1550 |
| n (Samp) | 1243 | 15 | 1243 | 21 | 1243 | 7 |
| n (Patient) | 397 | 15 | 397 | 21 | 397 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.63 | 0.61 | 0.71 | 0.66 | 0.82 | 0.69 | 0.56 | 0.70 | 0.55 |
| SE | 0.062 | 0.11 | 0.076 | 0.061 | 0.10 | 0.065 | 0.083 | 0.11 | 0.11 |
| p | 0.043 | 0.28 | 0.0067 | 0.0074 | 0.0021 | 0.0044 | 0.47 | 0.072 | 0.68 |
| nCohort 1 | 1385 | 1452 | 1243 | 1385 | 1452 | 1243 | 1385 | 1452 | 1243 |
| nCohort 2 | 24 | 8 | 15 | 24 | 6 | 21 | 13 | 7 | 7 |
| Cutoff 1 | 267 | 271 | 388 | 353 | 654 | 353 | 201 | 666 | 201 |
| Sens 1 | 71% | 75% | 73% | 71% | 83% | 71% | 77% | 71% | 71% |
| Spec 1 | 39% | 39% | 48% | 47% | 66% | 45% | 31% | 67% | 29% |
| Cutoff 2 | 185 | 108 | 259 | 245 | 654 | 313 | 77.5 | 395 | 77.5 |
| Sens 2 | 83% | 88% | 80% | 83% | 83% | 81% | 85% | 86% | 86% |
| Spec 2 | 30% | 20% | 36% | 37% | 66% | 41% | 15% | 49% | 13% |
| Cutoff 3 | 108 | 87.7 | 185 | 149 | 455 | 174 | 65.8 | 267 | 65.8 |
| Sens 3 | 92% | 100% | 93% | 92% | 100% | 90% | 92% | 100% | 100% |
| Spec 3 | 20% | 16% | 27% | 25% | 54% | 26% | 13% | 38% | 11% |
| Cutoff 4 | 726 | 751 | 750 | 726 | 751 | 750 | 726 | 751 | 750 |
| Sens 4 | 46% | 50% | 60% | 46% | 67% | 52% | 38% | 57% | 43% |
| Spec 4 | 70% | 70% | 70% | 70% | 70% | 70% | 70% | 70% | 70% |
| Cutoff 5 | 1020 | 1070 | 1040 | 1020 | 1070 | 1040 | 1020 | 1070 | 1040 |
| Sens 5 | 33% | 38% | 47% | 42% | 67% | 48% | 31% | 43% | 43% |
| Spec 5 | 80% | 80% | 80% | 80% | 80% | 80% | 80% | 80% | 80% |
| Cutoff 6 | 1470 | 1560 | 1470 | 1470 | 1560 | 1470 | 1470 | 1560 | 1470 |
| Sens 6 | 33% | 25% | 47% | 33% | 50% | 43% | 15% | 14% | 29% |
| Spec 6 | 90% | 90% | 90% | 90% | 90% | 90% | 90% | 90% | 90% |
| OR Quart 2 | 1.5 | 0.50 | 4.0 | 1.7 | >0 | 2.5 | 0.33 | >2.0 | 0.50 |
| p Value | 0.53 | 0.57 | 0.21 | 0.48 | <na | 0.27 | 0.34 | <0.57 | 0.57 |
| 95% CI of | 0.42 | 0.045 | 0.45 | 0.40 | >na | 0.49 | 0.034 | >0.18 | 0.045 |
| OR Quart 2 | 5.4 | 5.5 | 36 | 7.1 | na | 13 | 3.2 | na | 5.5 |
| OR Quart 3 | 0.75 | 0.50 | 1.0 | 1.7 | >2.0 | 1.5 | 1.3 | >2.0 | 0.50 |
| p Value | 0.71 | 0.57 | 1.0 | 0.48 | <0.57 | 0.66 | 0.70 | <0.57 | 0.57 |
| 95% CI of | 0.17 | 0.045 | 0.062 | 0.40 | >0.18 | 0.25 | 0.30 | >0.18 | 0.045 |
| OR Quart 3 | 3.4 | 5.5 | 16 | 7.1 | na | 9.1 | 6.0 | na | 5.5 |
| OR Quart 4 | 2.8 | 2.0 | 9.2 | 3.7 | >4.0 | 5.7 | 1.7 | >3.0 | 1.5 |
| p Value | 0.081 | 0.42 | 0.036 | 0.044 | <0.21 | 0.025 | 0.48 | <0.34 | 0.66 |
| 95% CI of | 0.88 | 0.37 | 1.2 | 1.0 | >0.45 | 1.2 | 0.40 | >0.31 | 0.25 |
| OR Quart 4 | 8.9 | 11 | 73 | 14 | na | 26 | 7.0 | na | 9.0 |

Immunoglogulin G1

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3300 | 10100 | 3300 | 13900 | 3300 | 5060 |
| Average | 5860 | 12600 | 5860 | 16000 | 5860 | 7690 |
| Stdev | 6930 | 9230 | 6930 | 16500 | 6930 | 7590 |
| p (t-test) | | 7.5E−6 | | 3.8E−10 | | 0.40 |
| Min | 3.36 | 980 | 3.36 | 1440 | 3.36 | 55.9 |
| Max | 80000 | 35700 | 80000 | 80000 | 80000 | 25300 |
| n (Samp) | 1268 | 22 | 1268 | 20 | 1268 | 10 |
| n (Patient) | 449 | 22 | 449 | 20 | 449 | 10 |
| sCr only | | | | | | |
| Median | 3480 | 10000 | nd | nd | nd | nd |
| Average | 6270 | 12600 | nd | nd | nd | nd |
| Stdev | 7490 | 9250 | nd | nd | nd | nd |
| p (t-test) | | 0.018 | nd | nd | nd | nd |
| Min | 3.36 | 980 | nd | nd | nd | nd |
| Max | 80000 | 28300 | nd | nd | nd | nd |
| n (Samp) | 1332 | 8 | nd | nd | nd | nd |
| n (Patient) | 464 | 8 | nd | nd | nd | nd |
| UO only | | | | | | |
| Median | 3420 | 11700 | 3420 | 14900 | 3420 | 5570 |
| Average | 5990 | 13400 | 5990 | 18400 | 5990 | 9330 |
| Stdev | 7020 | 9250 | 7020 | 17000 | 7020 | 8180 |
| p (t-test) | | 9.1E−5 | | 2.9E−13 | | 0.21 |
| Min | 3.36 | 1880 | 3.36 | 1440 | 3.36 | 2380 |
| Max | 80000 | 35700 | 80000 | 80000 | 80000 | 25300 |
| n (Samp) | 1114 | 14 | 1114 | 19 | 1114 | 7 |
| n (Patient) | 359 | 14 | 359 | 19 | 359 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.76 | 0.74 | 0.78 | 0.81 | nd | 0.84 | 0.60 | nd | 0.69 |
| SE | 0.060 | 0.10 | 0.074 | 0.059 | nd | 0.057 | 0.095 | nd | 0.11 |
| p | 1.2E−5 | 0.017 | 1.4E−4 | 1.5E−7 | nd | 2.0E−9 | 0.30 | nd | 0.086 |
| nCohort 1 | 1268 | 1332 | 1114 | 1268 | nd | 1114 | 1268 | nd | 1114 |
| nCohort 2 | 22 | 8 | 14 | 20 | nd | 19 | 10 | nd | 7 |
| Cutoff 1 | 6900 | 6900 | 8550 | 9500 | nd | 9500 | 3900 | nd | 4550 |
| Sens 1 | 73% | 75% | 71% | 70% | nd | 74% | 70% | nd | 71% |
| Spec 1 | 72% | 70% | 78% | 81% | nd | 81% | 56% | nd | 61% |
| Cutoff 2 | 5190 | 5190 | 4150 | 7030 | nd | 7030 | 2380 | nd | 3900 |
| Sens 2 | 82% | 88% | 86% | 80% | nd | 84% | 80% | nd | 86% |
| Spec 2 | 65% | 63% | 57% | 73% | nd | 72% | 37% | nd | 55% |
| Cutoff 3 | 1990 | 977 | 1990 | 4740 | nd | 4740 | 1740 | nd | 2380 |
| Sens 3 | 91% | 100% | 93% | 90% | nd | 95% | 90% | nd | 100% |
| Spec 3 | 31% | 12% | 30% | 63% | nd | 62% | 26% | nd | 36% |
| Cutoff 4 | 6240 | 6760 | 6490 | 6240 | nd | 6490 | 6240 | nd | 6490 |
| Sens 4 | 77% | 75% | 79% | 80% | nd | 84% | 40% | nd | 43% |
| Spec 4 | 70% | 70% | 70% | 70% | nd | 70% | 70% | nd | 70% |
| Cutoff 5 | 8950 | 9780 | 9100 | 8950 | nd | 9100 | 8950 | nd | 9100 |
| Sens 5 | 55% | 50% | 64% | 70% | nd | 74% | 30% | nd | 29% |
| Spec 5 | 80% | 80% | 80% | 80% | nd | 80% | 80% | nd | 80% |
| Cutoff 6 | 14200 | 15400 | 14300 | 14200 | nd | 14300 | 14200 | nd | 14300 |
| Sens 6 | 36% | 38% | 43% | 50% | nd | 58% | 20% | nd | 29% |
| Spec 6 | 90% | 90% | 90% | 90% | nd | 90% | 90% | nd | 90% |
| OR Quart 2 | 2.0 | 0 | >2.0 | 1.0 | nd | 0 | 2.0 | nd | >1.0 |
| p Value | 0.57 | na | <0.57 | 1.0 | nd | na | 0.57 | nd | <1.00 |
| 95% CI of | 0.18 | na | >0.18 | 0.062 | nd | na | 0.18 | nd | >0.062 |
| OR Quart 2 | 22 | na | na | 16 | nd | na | 22 | nd | na |
| OR Quart 3 | 4.0 | 2.0 | >1.0 | 3.0 | nd | 3.0 | 3.0 | nd | >3.0 |
| p Value | 0.21 | 0.57 | <1.00 | 0.34 | nd | 0.34 | 0.34 | nd | <0.34 |
| 95% CI of | 0.45 | 0.18 | >0.062 | 0.31 | nd | 0.31 | 0.31 | nd | >0.31 |
| OR Quart 3 | 36 | 22 | na | 29 | nd | 29 | 29 | nd | na |
| OR Quart 4 | 16 | 5.1 | >11 | 16 | nd | 16 | 4.0 | nd | >3.0 |
| p Value | 0.0080 | 0.14 | <0.020 | 0.0079 | nd | 0.0078 | 0.21 | nd | <0.34 |
| 95% CI of | 2.1 | 0.59 | >1.5 | 2.1 | nd | 2.1 | 0.45 | nd | >0.31 |
| OR Quart 4 | 120 | 44 | na | 120 | nd | 120 | 36 | nd | na |

Immunoglogulin G2

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 9080 | 24500 | 9080 | 31300 | 9080 | 11300 |
| Average | 18400 | 51400 | 18400 | 57600 | 18400 | 42800 |
| Stdev | 31600 | 63700 | 31600 | 80400 | 31600 | 73900 |
| p (t-test) | | 2.5E−6 | | 1.5E−7 | | 0.017 |
| Min | 119 | 2380 | 119 | 3940 | 119 | 25.4 |
| Max | 240000 | 240000 | 240000 | 240000 | 240000 | 240000 |
| n (Samp) | 1268 | 22 | 1268 | 20 | 1268 | 10 |
| n (Patient) | 449 | 22 | 449 | 20 | 449 | 10 |
| sCr only | | | | | | |
| Median | 9340 | 24600 | nd | nd | nd | nd |
| Average | 20400 | 55100 | nd | nd | nd | nd |
| Stdev | 36100 | 62200 | nd | nd | nd | nd |
| p (t-test) | | 0.0071 | nd | nd | nd | nd |
| Min | 119 | 2380 | nd | nd | nd | nd |
| Max | 240000 | 182000 | nd | nd | nd | nd |
| n (Samp) | 1332 | 8 | nd | nd | nd | nd |
| n (Patient) | 464 | 8 | nd | nd | nd | nd |
| UO only | | | | | | |
| Median | 9630 | 24500 | 9630 | 33000 | 9630 | 13800 |
| Average | 18500 | 63500 | 18500 | 76200 | 18500 | 52800 |
| Stdev | 30500 | 81000 | 30500 | 89800 | 30500 | 86800 |
| p (t-test) | | 1.5E−7 | | 2.9E−14 | | 0.0038 |
| Min | 25.4 | 4460 | 25.4 | 3940 | 25.4 | 2320 |
| Max | 240000 | 240000 | 240000 | 240000 | 240000 | 240000 |
| n (Samp) | 1114 | 14 | 1114 | 19 | 1114 | 7 |
| n (Patient) | 359 | 14 | 359 | 19 | 359 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.71 | 0.73 | 0.71 | 0.74 | nd | 0.77 | 0.57 | nd | 0.61 |
| SE | 0.063 | 0.10 | 0.078 | 0.064 | nd | 0.064 | 0.095 | nd | 0.11 |
| p | 0.0011 | 0.023 | 0.0064 | 1.5E−4 | nd | 1.9E−5 | 0.48 | nd | 0.32 |
| nCohort 1 | 1268 | 1332 | 1114 | 1268 | nd | 1114 | 1268 | nd | 1114 |
| nCohort 2 | 22 | 8 | 14 | 20 | nd | 19 | 10 | nd | 7 |
| Cutoff 1 | 11800 | 13700 | 13700 | 14100 | nd | 14100 | 8540 | nd | 8840 |
| Sens 1 | 73% | 75% | 71% | 70% | nd | 74% | 70% | nd | 71% |
| Spec 1 | 60% | 65% | 65% | 67% | nd | 66% | 47% | nd | 47% |
| Cutoff 2 | 5910 | 11800 | 5910 | 12400 | nd | 12400 | 6890 | nd | 8540 |
| Sens 2 | 82% | 88% | 86% | 80% | nd | 84% | 80% | nd | 86% |
| Spec 2 | 31% | 59% | 29% | 63% | nd | 61% | 37% | nd | 45% |
| Cutoff 3 | 4910 | 2370 | 5180 | 5300 | nd | 5300 | 2270 | nd | 2270 |
| Sens 3 | 91% | 100% | 93% | 90% | nd | 95% | 90% | nd | 100% |
| Spec 3 | 25% | 8% | 24% | 27% | nd | 25% | 9% | nd | 8% |
| Cutoff 4 | 15200 | 16200 | 16000 | 15200 | nd | 16000 | 15200 | nd | 16000 |
| Sens 4 | 59% | 62% | 57% | 60% | nd | 63% | 30% | nd | 29% |
| Spec 4 | 70% | 70% | 70% | 70% | nd | 70% | 70% | nd | 70% |
| Cutoff 5 | 21200 | 22500 | 21800 | 21200 | nd | 21800 | 21200 | nd | 21800 |
| Sens 5 | 59% | 62% | 57% | 55% | nd | 58% | 30% | nd | 29% |
| Spec 5 | 80% | 80% | 80% | 80% | nd | 80% | 80% | nd | 80% |
| Cutoff 6 | 37600 | 41400 | 37600 | 37600 | nd | 37600 | 37600 | nd | 37600 |
| Sens 6 | 36% | 38% | 43% | 30% | nd | 42% | 30% | nd | 29% |
| Spec 6 | 90% | 90% | 90% | 90% | nd | 90% | 90% | nd | 90% |
| OR Quart 2 | 0.66 | 0 | 0.50 | 1.0 | nd | 2.0 | 1.5 | nd | 2.0 |
| p Value | 0.65 | na | 0.57 | 1.0 | nd | 0.57 | 0.66 | nd | 0.57 |
| 95% CI of | 0.11 | na | 0.045 | 0.14 | nd | 0.18 | 0.25 | nd | 0.18 |
| OR Quart 2 | 4.0 | na | 5.5 | 7.1 | nd | 22 | 9.0 | nd | 22 |
| OR Quart 3 | 1.3 | 2.0 | 1.5 | 2.0 | nd | 4.0 | 1.0 | nd | 2.0 |
| p Value | 0.70 | 0.57 | 0.66 | 0.42 | nd | 0.21 | 1.0 | nd | 0.57 |
| 95% CI of | 0.30 | 0.18 | 0.25 | 0.37 | nd | 0.45 | 0.14 | nd | 0.18 |
| OR Quart 3 | 6.0 | 22 | 9.1 | 11 | nd | 36 | 7.1 | nd | 22 |
| OR Quart 4 | 4.5 | 5.1 | 4.1 | 6.2 | nd | 12 | 1.5 | nd | 2.0 |
| p Value | 0.021 | 0.14 | 0.077 | 0.018 | nd | 0.016 | 0.66 | nd | 0.57 |
| 95% CI of | 1.3 | 0.59 | 0.86 | 1.4 | nd | 1.6 | 0.25 | nd | 0.18 |
| OR Quart 4 | 16 | 44 | 19 | 28 | nd | 96 | 9.0 | nd | 22 |

Interleukin-11

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 133 | 222 | 133 | 253 | 133 | 93.2 |
| Average | 212 | 502 | 212 | 422 | 212 | 327 |
| Stdev | 234 | 693 | 234 | 570 | 234 | 645 |
| p (t-test) | | 6.8E−8 | | 1.8E−4 | | 0.13 |
| Min | 0.0822 | 48.0 | 0.0822 | 49.5 | 0.0822 | 14.5 |
| Max | 2260 | 2900 | 2260 | 2590 | 2260 | 2140 |
| n (Samp) | 1275 | 22 | 1275 | 19 | 1275 | 10 |
| n (Patient) | 452 | 22 | 452 | 19 | 452 | 10 |
| sCr only | | | | | | |
| Median | 137 | 124 | nd | nd | nd | nd |
| Average | 231 | 276 | nd | nd | nd | nd |
| Stdev | 302 | 348 | nd | nd | nd | nd |
| p (t-test) | | 0.68 | nd | nd | nd | nd |
| Min | 0.0822 | 48.0 | nd | nd | nd | nd |
| Max | 3780 | 1040 | nd | nd | nd | nd |
| n (Samp) | 1338 | 8 | nd | nd | nd | nd |
| n (Patient) | 467 | 8 | nd | nd | nd | nd |
| UO only | | | | | | |
| Median | 142 | 384 | 142 | 255 | 142 | 95.5 |
| Average | 220 | 588 | 220 | 490 | 220 | 430 |
| Stdev | 238 | 740 | 238 | 623 | 238 | 763 |
| p (t-test) | | 5.4E−8 | | 5.2E−6 | | 0.023 |
| Min | 0.0822 | 116 | 0.0822 | 49.5 | 0.0822 | 14.5 |
| Max | 2260 | 2900 | 2260 | 2590 | 2260 | 2140 |
| n (Samp) | 1121 | 14 | 1121 | 18 | 1121 | 7 |
| n (Patient) | 362 | 14 | 362 | 18 | 362 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.68 | 0.51 | 0.75 | 0.69 | nd | 0.69 | 0.46 | nd | 0.50 |
| SE | 0.064 | 0.10 | 0.076 | 0.068 | nd | 0.070 | 0.094 | nd | 0.11 |
| p | 0.0046 | 0.93 | 0.0012 | 0.0043 | nd | 0.0068 | 0.63 | nd | 0.98 |
| nCohort 1 | 1275 | 1338 | 1121 | 1275 | nd | 1121 | 1275 | nd | 1121 |
| nCohort 2 | 22 | 8 | 14 | 19 | nd | 18 | 10 | nd | 7 |
| Cutoff 1 | 131 | 87.1 | 158 | 181 | nd | 179 | 88.1 | nd | 90.5 |
| Sens 1 | 73% | 75% | 71% | 74% | nd | 72% | 70% | nd | 71% |
| Spec 1 | 49% | 32% | 55% | 63% | nd | 60% | 33% | nd | 31% |
| Cutoff 2 | 115 | 76.3 | 131 | 135 | nd | 135 | 85.7 | nd | 85.7 |
| Sens 2 | 82% | 88% | 86% | 84% | nd | 83% | 80% | nd | 86% |
| Spec 2 | 44% | 26% | 46% | 51% | nd | 48% | 32% | nd | 29% |
| Cutoff 3 | 87.1 | 47.7 | 128 | 79.7 | nd | 79.7 | 66.2 | nd | 13.5 |
| Sens 3 | 91% | 100% | 93% | 95% | nd | 94% | 90% | nd | 100% |
| Spec 3 | 32% | 13% | 46% | 29% | nd | 27% | 23% | nd | 1% |
| Cutoff 4 | 226 | 235 | 235 | 226 | nd | 235 | 226 | nd | 235 |
| Sens 4 | 50% | 25% | 57% | 58% | nd | 61% | 30% | nd | 43% |
| Spec 4 | 70% | 70% | 70% | 70% | nd | 70% | 70% | nd | 70% |
| Cutoff 5 | 312 | 329 | 319 | 312 | nd | 319 | 312 | nd | 319 |
| Sens 5 | 45% | 25% | 57% | 32% | nd | 33% | 20% | nd | 29% |
| Spec 5 | 80% | 80% | 80% | 80% | nd | 80% | 80% | nd | 80% |
| Cutoff 6 | 469 | 503 | 474 | 469 | nd | 474 | 469 | nd | 474 |
| Sens 6 | 32% | 25% | 43% | 21% | nd | 28% | 10% | nd | 14% |
| Spec 6 | 90% | 90% | 90% | 90% | nd | 90% | 90% | nd | 90% |
| OR Quart 2 | 6.1 | 4.0 | >3.0 | 2.0 | nd | 4.0 | 0.50 | nd | 0.50 |
| p Value | 0.095 | 0.21 | <0.34 | 0.57 | nd | 0.21 | 0.57 | nd | 0.57 |
| 95% CI of | 0.73 | 0.45 | >0.31 | 0.18 | nd | 0.45 | 0.045 | nd | 0.045 |
| OR Quart 2 | 51 | 36 | na | 22 | nd | 36 | 5.5 | nd | 5.5 |
| OR Quart 3 | 5.1 | 1.0 | >3.0 | 8.2 | nd | 5.1 | 2.5 | nd | 1.5 |
| p Value | 0.14 | 1.0 | <0.34 | 0.048 | nd | 0.14 | 0.27 | nd | 0.66 |
| 95% CI of | 0.59 | 0.062 | >0.31 | 1.0 | nd | 0.59 | 0.49 | nd | 0.25 |
| OR Quart 3 | 44 | 16 | na | 66 | nd | 44 | 13 | nd | 9.1 |
| OR Quart 4 | 10 | 2.0 | >8.2 | 8.2 | nd | 8.2 | 1.0 | nd | 0.50 |
| p Value | 0.027 | 0.57 | <0.048 | 0.049 | nd | 0.048 | 1.00 | nd | 0.57 |
| 95% CI of | 1.3 | 0.18 | >1.0 | 1.0 | nd | 1.0 | 0.14 | nd | 0.045 |
| OR Quart 4 | 81 | 22 | na | 66 | nd | 66 | 7.2 | nd | 5.5 |

Interleukin-2 receptor alpha chain

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 573 | 935 | 573 | 1390 | 573 | 770 |
| Average | 1010 | 1690 | 1010 | 2110 | 1010 | 1620 |
| Stdev | 1270 | 1810 | 1270 | 2430 | 1270 | 2400 |
| p (t-test) | | 0.014 | | 1.6E−4 | | 0.13 |
| Min | 0.0317 | 0.118 | 0.0317 | 50.9 | 0.0317 | 0.115 |
| Max | 10400 | 6080 | 10400 | 9090 | 10400 | 7740 |
| n (Samp) | 1277 | 22 | 1277 | 20 | 1277 | 10 |
| n (Patient) | 452 | 22 | 452 | 20 | 452 | 10 |
| sCr only | | | | | | |
| Median | 609 | 485 | nd | nd | nd | nd |
| Average | 1090 | 1290 | nd | nd | nd | nd |
| Stdev | 1420 | 2030 | nd | nd | nd | nd |
| p (t-test) | | 0.70 | nd | nd | nd | nd |
| Min | 0.0317 | 0.118 | nd | nd | nd | nd |
| Max | 10400 | 6080 | nd | nd | nd | nd |
| n (Samp) | 1341 | 8 | nd | nd | nd | nd |
| n (Patient) | 467 | 8 | nd | nd | nd | nd |
| UO only | | | | | | |
| Median | 624 | 1540 | 624 | 1700 | 624 | 819 |
| Average | 1050 | 2130 | 1050 | 2300 | 1050 | 2120 |
| Stdev | 1280 | 1940 | 1280 | 2450 | 1280 | 2740 |
| p (t-test) | | 0.0020 | | 3.7E−5 | | 0.029 |
| Min | 0.0317 | 54.4 | 0.0317 | 50.9 | 0.0317 | 19.9 |
| Max | 10400 | 6080 | 10400 | 9090 | 10400 | 7740 |
| n (Samp) | 1124 | 14 | 1124 | 19 | 1124 | 7 |
| n (Patient) | 362 | 14 | 362 | 19 | 362 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.59 | 0.47 | 0.66 | 0.65 | nd | 0.68 | 0.52 | nd | 0.58 |
| SE | 0.064 | 0.10 | 0.080 | 0.067 | nd | 0.068 | 0.093 | nd | 0.11 |
| p | 0.16 | 0.78 | 0.052 | 0.021 | nd | 0.0078 | 0.86 | nd | 0.46 |
| nCohort 1 | 1277 | 1341 | 1124 | 1277 | nd | 1124 | 1277 | nd | 1124 |
| nCohort 2 | 22 | 8 | 14 | 20 | nd | 19 | 10 | nd | 7 |
| Cutoff 1 | 350 | 189 | 477 | 761 | nd | 761 | 227 | nd | 718 |
| Sens 1 | 73% | 75% | 71% | 70% | nd | 74% | 70% | nd | 71% |
| Spec 1 | 37% | 24% | 42% | 57% | nd | 56% | 28% | nd | 54% |
| Cutoff 2 | 189 | 60.5 | 277 | 385 | nd | 385 | 35.7 | nd | 35.7 |
| Sens 2 | 82% | 88% | 86% | 80% | nd | 84% | 80% | nd | 86% |
| Spec 2 | 25% | 12% | 30% | 39% | nd | 37% | 10% | nd | 9% |
| Cutoff 3 | 60.5 | 0.116 | 89.0 | 69.2 | nd | 68.0 | 18.6 | nd | 18.6 |
| Sens 3 | 91% | 100% | 93% | 90% | nd | 95% | 90% | nd | 100% |
| Spec 3 | 13% | 2% | 13% | 14% | nd | 12% | 8% | nd | 7% |
| Cutoff 4 | 1110 | 1160 | 1140 | 1110 | nd | 1140 | 1110 | nd | 1140 |
| Sens 4 | 45% | 38% | 57% | 55% | nd | 58% | 40% | nd | 43% |
| Spec 4 | 70% | 70% | 70% | 70% | nd | 70% | 70% | nd | 70% |
| Cutoff 5 | 1620 | 1760 | 1670 | 1620 | nd | 1670 | 1620 | nd | 1670 |
| Sens 5 | 36% | 12% | 50% | 45% | nd | 53% | 30% | nd | 43% |
| Spec 5 | 80% | 80% | 80% | 80% | nd | 80% | 80% | nd | 80% |
| Cutoff 6 | 2420 | 2620 | 2480 | 2420 | nd | 2480 | 2420 | nd | 2480 |
| Sens 6 | 32% | 12% | 43% | 30% | nd | 37% | 20% | nd | 29% |
| Spec 6 | 90% | 90% | 90% | 90% | nd | 90% | 90% | nd | 90% |
| OR Quart 2 | 1.00 | 0.50 | 1.5 | 0.50 | nd | 0.66 | 0.33 | nd | 0 |
| p Value | 1.00 | 0.57 | 0.66 | 0.42 | nd | 0.65 | 0.34 | nd | na |
| 95% CI of | 0.29 | 0.045 | 0.25 | 0.090 | nd | 0.11 | 0.034 | nd | na |
| OR Quart 2 | 3.5 | 5.5 | 9.0 | 2.7 | nd | 4.0 | 3.2 | nd | na |
| OR Quart 3 | 0.80 | 1.0 | 1.0 | 1.0 | nd | 1.3 | 1.00 | nd | 1.00 |
| p Value | 0.73 | 1.00 | 1.0 | 1.0 | nd | 0.71 | 1.00 | nd | 1.00 |
| 95% CI of | 0.21 | 0.14 | 0.14 | 0.25 | nd | 0.30 | 0.20 | nd | 0.14 |
| OR Quart 3 | 3.0 | 7.2 | 7.1 | 4.0 | nd | 6.0 | 5.0 | nd | 7.1 |
| OR Quart 4 | 1.6 | 1.5 | 3.6 | 2.5 | nd | 3.4 | 1.00 | nd | 1.5 |
| p Value | 0.41 | 0.65 | 0.12 | 0.12 | nd | 0.065 | 1.00 | nd | 0.66 |
| 95% CI of | 0.52 | 0.25 | 0.73 | 0.79 | nd | 0.93 | 0.20 | nd | 0.25 |
| OR Quart 4 | 5.0 | 9.1 | 17 | 8.2 | nd | 13 | 5.0 | nd | 9.0 |

Neutrophil collagenase

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3540 | 13400 | 3540 | 13400 | 3540 | 3260 |
| Average | 15000 | 81600 | 15000 | 38600 | 15000 | 11700 |
| Stdev | 41900 | 159000 | 41900 | 49400 | 41900 | 18300 |
| p (t-test) | | 4.3E−12 | | 0.0066 | | 0.78 |
| Min | 0.114 | 0.260 | 0.114 | 25.8 | 0.114 | 212 |
| Max | 670000 | 649000 | 670000 | 166000 | 670000 | 66900 |
| n (Samp) | 1382 | 24 | 1382 | 24 | 1382 | 13 |
| n (Patient) | 485 | 24 | 485 | 24 | 485 | 13 |
| sCr only | | | | | | |
| Median | 3770 | 5520 | 3770 | 14100 | 3770 | 10800 |
| Average | 17100 | 99700 | 17100 | 97000 | 17100 | 10600 |
| Stdev | 47900 | 225000 | 47900 | 160000 | 47900 | 8750 |
| p (t-test) | | 3.9E−6 | | 6.4E−5 | | 0.72 |
| Min | 0.114 | 0.260 | 0.114 | 25.8 | 0.114 | 1120 |
| Max | 670000 | 649000 | 670000 | 401000 | 670000 | 21900 |
| n (Samp) | 1449 | 8 | 1449 | 6 | 1449 | 7 |
| n (Patient) | 501 | 8 | 501 | 6 | 501 | 7 |
| UO only | | | | | | |
| Median | 3590 | 22500 | 3590 | 20500 | 3590 | 3260 |
| Average | 14600 | 80500 | 14600 | 61600 | 14600 | 13500 |
| Stdev | 38300 | 147000 | 38300 | 82900 | 38300 | 24100 |
| p (t-test) | | 9.7E−10 | | 7.0E−8 | | 0.94 |
| Min | 0.114 | 1660 | 0.114 | 551 | 0.114 | 576 |
| Max | 670000 | 561000 | 670000 | 327000 | 670000 | 66900 |
| n (Samp) | 1240 | 15 | 1240 | 21 | 1240 | 7 |
| n (Patient) | 398 | 15 | 398 | 21 | 398 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.68 | 0.55 | 0.76 | 0.72 | 0.61 | 0.77 | 0.51 | 0.61 | 0.49 |
| SE | 0.061 | 0.11 | 0.072 | 0.060 | 0.12 | 0.061 | 0.081 | 0.11 | 0.11 |
| p | 0.0024 | 0.64 | 2.5E−4 | 3.1E−4 | 0.38 | 6.5E−6 | 0.90 | 0.34 | 0.95 |
| nCohort 1 | 1382 | 1449 | 1240 | 1382 | 1449 | 1240 | 1382 | 1449 | 1240 |
| nCohort 2 | 24 | 8 | 15 | 24 | 6 | 21 | 13 | 7 | 7 |
| Cutoff 1 | 5080 | 3020 | 8450 | 7290 | 978 | 10400 | 1120 | 2970 | 1430 |
| Sens 1 | 71% | 75% | 73% | 71% | 83% | 71% | 77% | 71% | 71% |
| Spec 1 | 58% | 45% | 69% | 65% | 22% | 74% | 25% | 44% | 29% |
| Cutoff 2 | 2970 | 127 | 5080 | 3090 | 978 | 7290 | 576 | 2660 | 576 |
| Sens 2 | 83% | 88% | 80% | 83% | 83% | 81% | 85% | 86% | 86% |
| Spec 2 | 46% | 3% | 58% | 46% | 22% | 65% | 15% | 42% | 15% |
| Cutoff 3 | 1650 | 0.159 | 2030 | 2450 | 23.8 | 2660 | 571 | 1120 | 571 |
| Sens 3 | 92% | 100% | 93% | 92% | 100% | 90% | 92% | 100% | 100% |
| Spec 3 | 34% | 0% | 36% | 42% | 1% | 43% | 15% | 24% | 15% |
| Cutoff 4 | 8980 | 9730 | 8740 | 8980 | 9730 | 8740 | 8980 | 9730 | 8740 |
| Sens 4 | 54% | 38% | 67% | 62% | 50% | 71% | 38% | 57% | 29% |
| Spec 4 | 70% | 70% | 70% | 70% | 70% | 70% | 70% | 70% | 70% |
| Cutoff 5 | 15600 | 17000 | 15100 | 15600 | 17000 | 15100 | 15600 | 17000 | 15100 |
| Sens 5 | 46% | 25% | 53% | 46% | 50% | 52% | 23% | 29% | 14% |
| Spec 5 | 80% | 80% | 80% | 80% | 80% | 80% | 80% | 80% | 80% |
| Cutoff 6 | 32700 | 36000 | 32700 | 32700 | 36000 | 32700 | 32700 | 36000 | 32700 |
| Sens 6 | 33% | 25% | 40% | 33% | 33% | 43% | 8% | 0% | 14% |
| Spec 6 | 90% | 90% | 90% | 90% | 90% | 90% | 90% | 90% | 90% |
| OR Quart 2 | 2.5 | 0.50 | >3.0 | 2.0 | 0 | 3.0 | 0.75 | 2.0 | 0.50 |
| p Value | 0.27 | 0.57 | <0.34 | 0.42 | na | 0.34 | 0.70 | 0.57 | 0.57 |
| 95% CI of | 0.48 | 0.045 | >0.31 | 0.36 | na | 0.31 | 0.17 | 0.18 | 0.045 |
| OR Quart 2 | 13 | 5.5 | na | 11 | na | 29 | 3.4 | 22 | 5.5 |
| OR Quart 3 | 2.0 | 1.0 | >2.0 | 2.0 | 0.50 | 3.0 | 0.50 | 1.0 | 1.0 |
| p Value | 0.42 | 1.0 | <0.57 | 0.42 | 0.57 | 0.34 | 0.42 | 1.0 | 1.0 |
| 95% CI of | 0.37 | 0.14 | >0.18 | 0.37 | 0.045 | 0.31 | 0.090 | 0.062 | 0.14 |
| OR Quart 3 | 11 | 7.1 | na | 11 | 5.5 | 29 | 2.7 | 16 | 7.1 |
| OR Quart 4 | 6.7 | 1.5 | >10 | 7.2 | 1.5 | 15 | 1.00 | 3.0 | 1.0 |
| p Value | 0.013 | 0.66 | <0.027 | 0.0092 | 0.66 | 0.0099 | 1.00 | 0.34 | 1.00 |
| 95% CI of | 1.5 | 0.25 | >1.3 | 1.6 | 0.25 | 1.9 | 0.25 | 0.31 | 0.14 |
| OR Quart 4 | 30 | 9.0 | na | 32 | 9.0 | 110 | 4.0 | 29 | 7.2 |

Protransforming growth factor alpha

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 6.25 | 13.8 | 6.25 | 12.5 | 6.25 | 13.5 |
| Average | 10.9 | 20.9 | 10.9 | 20.1 | 10.9 | 14.9 |
| Stdev | 21.2 | 26.1 | 21.2 | 16.0 | 21.2 | 12.1 |
| p (t-test) | | 0.028 | | 0.051 | | 0.54 |
| Min | 0.00184 | 0.781 | 0.00184 | 3.26 | 0.00184 | 0.00454 |
| Max | 361 | 122 | 361 | 50.4 | 361 | 36.0 |
| n (Samp) | 1271 | 22 | 1271 | 20 | 1271 | 10 |
| n (Patient) | 452 | 22 | 452 | 20 | 452 | 10 |
| sCr only | | | | | | |
| Median | 6.57 | 13.7 | nd | nd | nd | nd |
| Average | 11.3 | 12.4 | nd | nd | nd | nd |
| Stdev | 21.2 | 5.27 | nd | nd | nd | nd |
| p (t-test) | | 0.89 | nd | nd | nd | nd |
| Min | 0.00184 | 1.41 | nd | nd | nd | nd |
| Max | 361 | 18.0 | nd | nd | nd | nd |
| n (Samp) | 1335 | 8 | nd | nd | nd | nd |
| n (Patient) | 467 | 8 | nd | nd | nd | nd |
| UO only | | | | | | |
| Median | 6.50 | 16.0 | 6.50 | 17.8 | 6.50 | 14.4 |
| Average | 11.3 | 21.8 | 11.3 | 21.7 | 11.3 | 17.6 |
| Stdev | 22.3 | 19.7 | 22.3 | 15.6 | 22.3 | 13.1 |
| p (t-test) | | 0.080 | | 0.044 | | 0.45 |
| Min | 0.00184 | 0.781 | 0.00184 | 3.26 | 0.00184 | 0.564 |
| Max | 361 | 63.1 | 361 | 50.4 | 361 | 36.0 |
| n (Samp) | 1118 | 14 | 1118 | 19 | 1118 | 7 |
| n (Patient) | 362 | 14 | 362 | 19 | 362 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.72 | 0.70 | 0.73 | 0.74 | nd | 0.78 | 0.65 | nd | 0.70 |
| SE | 0.062 | 0.10 | 0.078 | 0.064 | nd | 0.064 | 0.095 | nd | 0.11 |
| p | 3.3E−4 | 0.061 | 0.0034 | 1.8E−4 | nd | 1.4E−5 | 0.12 | nd | 0.069 |
| nCohort 1 | 1271 | 1335 | 1118 | 1271 | nd | 1118 | 1271 | nd | 1118 |
| nCohort 2 | 22 | 8 | 14 | 20 | nd | 19 | 10 | nd | 7 |
| Cutoff 1 | 9.29 | 11.3 | 8.44 | 10.5 | nd | 11.3 | 9.53 | nd | 12.6 |
| Sens 1 | 73% | 75% | 71% | 70% | nd | 74% | 70% | nd | 71% |
| Spec 1 | 67% | 73% | 63% | 72% | nd | 74% | 68% | nd | 78% |
| Cutoff 2 | 6.62 | 9.29 | 6.53 | 6.37 | nd | 8.14 | 6.20 | nd | 6.20 |
| Sens 2 | 82% | 88% | 86% | 80% | nd | 84% | 80% | nd | 86% |
| Spec 2 | 52% | 65% | 50% | 51% | nd | 62% | 50% | nd | 49% |
| Cutoff 3 | 5.52 | 1.41 | 5.48 | 4.33 | nd | 4.33 | 0.530 | nd | 0.530 |
| Sens 3 | 91% | 100% | 93% | 90% | nd | 95% | 90% | nd | 100% |
| Spec 3 | 45% | 10% | 44% | 35% | nd | 34% | 5% | nd | 5% |
| Cutoff 4 | 9.95 | 10.5 | 10.1 | 9.95 | nd | 10.1 | 9.95 | nd | 10.1 |
| Sens 4 | 68% | 75% | 64% | 70% | nd | 79% | 60% | nd | 71% |
| Spec 4 | 70% | 70% | 70% | 70% | nd | 70% | 70% | nd | 70% |
| Cutoff 5 | 13.4 | 14.1 | 13.7 | 13.4 | nd | 13.7 | 13.4 | nd | 13.7 |
| Sens 5 | 55% | 50% | 57% | 45% | nd | 53% | 50% | nd | 57% |
| Spec 5 | 80% | 80% | 80% | 80% | nd | 80% | 80% | nd | 80% |
| Cutoff 6 | 21.9 | 23.3 | 23.2 | 21.9 | nd | 23.2 | 21.9 | nd | 23.2 |
| Sens 6 | 18% | 0% | 29% | 35% | nd | 37% | 20% | nd | 29% |
| Spec 6 | 90% | 90% | 90% | 90% | nd | 90% | 90% | nd | 90% |
| OR Quart 2 | 0.50 | 0 | 1.0 | >4.0 | nd | >3.0 | 0.50 | nd | 1.0 |
| p Value | 0.57 | na | 1.0 | <0.21 | nd | <0.34 | 0.57 | nd | 1.0 |
| 95% CI of | 0.045 | na | 0.062 | >0.45 | nd | >0.31 | 0.045 | nd | 0.062 |
| OR Quart 2 | 5.5 | na | 16 | na | nd | na | 5.5 | nd | 16 |
| OR Quart 3 | 3.0 | 2.0 | 4.0 | >5.1 | nd | >4.1 | 0.50 | nd | 0 |
| p Value | 0.18 | 0.57 | 0.21 | <0.14 | nd | <0.21 | 0.57 | nd | na |
| 95% CI of | 0.61 | 0.18 | 0.45 | >0.59 | nd | >0.45 | 0.045 | nd | na |
| OR Quart 3 | 15 | 22 | 36 | na | nd | na | 5.5 | nd | na |
| OR Quart 4 | 6.7 | 5.0 | 8.2 | >11 | nd | >12 | 3.0 | nd | 5.1 |
| p Value | 0.013 | 0.14 | 0.048 | <0.020 | nd | <0.016 | 0.18 | nd | 0.14 |
| 95% CI of | 1.5 | 0.59 | 1.0 | >1.5 | nd | >1.6 | 0.61 | nd | 0.59 |
| OR Quart 4 | 30 | 43 | 66 | na | nd | na | 15 | nd | 44 |

CA 15-3

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 434 | 434 | 434 | 434 | 434 | 434 |
| Average | 326 | 423 | 326 | 325 | 326 | 308 |
| Stdev | 223 | 180 | 223 | 224 | 223 | 191 |
| p (t-test) | | 0.17 | | 0.99 | | 0.82 |
| Min | 1.17 | 22.0 | 1.17 | 39.8 | 1.17 | 4.72 |
| Max | 784 | 784 | 784 | 784 | 784 | 434 |
| n (Samp) | 558 | 10 | 558 | 15 | 558 | 8 |
| n (Patient) | 262 | 10 | 262 | 15 | 262 | 8 |
| sCr only | | | | | | |
| Median | nd | nd | nd | nd | 434 | 434 |
| Average | nd | nd | nd | nd | 326 | 406 |
| Stdev | nd | nd | nd | nd | 223 | 68.4 |
| p (t-test) | nd | nd | nd | nd | | 0.38 |
| Min | nd | nd | nd | nd | 1.17 | 266 |
| Max | nd | nd | nd | nd | 784 | 434 |
| n (Samp) | nd | nd | nd | nd | 575 | 6 |
| n (Patient) | nd | nd | nd | nd | 269 | 6 |
| UO only | | | | | | |
| Median | nd | nd | 434 | 434 | nd | nd |
| Average | nd | nd | 331 | 349 | nd | nd |
| Stdev | nd | nd | 218 | 235 | nd | nd |
| p (t-test) | nd | nd | | 0.80 | nd | nd |
| Min | nd | nd | 1.17 | 52.2 | nd | nd |
| Max | nd | nd | 784 | 784 | nd | nd |
| n (Samp) | nd | nd | 533 | 10 | nd | nd |
| n (Patient) | nd | nd | 235 | 10 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.60 | nd | nd | 0.53 | nd | 0.55 | 0.47 | 0.60 | nd |
| SE | 0.096 | nd | nd | 0.077 | nd | 0.095 | 0.10 | 0.12 | nd |
| p | 0.28 | nd | nd | 0.71 | nd | 0.58 | 0.75 | 0.40 | nd |
| nCohort 1 | 558 | nd | nd | 558 | nd | 533 | 558 | 575 | nd |
| nCohort 2 | 10 | nd | nd | 15 | nd | 10 | 8 | 6 | nd |
| Cutoff 1 | 427 | nd | nd | 88.4 | nd | 239 | 265 | 427 | nd |
| Sens 1 | 80% | nd | nd | 73% | nd | 70% | 75% | 83% | nd |
| Spec 1 | 39% | nd | nd | 28% | nd | 33% | 36% | 39% | nd |
| Cutoff 2 | 427 | nd | nd | 62.5 | nd | 87.5 | 19.7 | 427 | nd |
| Sens 2 | 80% | nd | nd | 80% | nd | 80% | 88% | 83% | nd |
| Spec 2 | 39% | nd | nd | 24% | nd | 26% | 10% | 39% | nd |
| Cutoff 3 | 387 | nd | nd | 45.0 | nd | 62.5 | 4.68 | 265 | nd |
| Sens 3 | 90% | nd | nd | 93% | nd | 90% | 100% | 100% | nd |
| Spec 3 | 39% | nd | nd | 20% | nd | 23% | 1% | 36% | nd |
| Cutoff 4 | 434 | nd | nd | 434 | nd | 434 | 434 | 434 | nd |
| Sens 4 | 10% | nd | nd | 13% | nd | 20% | 0% | 0% | nd |
| Spec 4 | 92% | nd | nd | 92% | nd | 92% | 92% | 91% | nd |
| Cutoff 5 | 434 | nd | nd | 434 | nd | 434 | 434 | 434 | nd |
| Sens 5 | 10% | nd | nd | 13% | nd | 20% | 0% | 0% | nd |
| Spec 5 | 92% | nd | nd | 92% | nd | 92% | 92% | 91% | nd |
| Cutoff 6 | 434 | nd | nd | 434 | nd | 434 | 434 | 434 | nd |
| Sens 6 | 10% | nd | nd | 13% | nd | 20% | 0% | 0% | nd |
| Spec 6 | 92% | nd | nd | 92% | nd | 92% | 92% | 91% | nd |
| OR Quart 2 | 8.4 | nd | nd | 0.49 | nd | 0.99 | >0 | >6.3 | nd |
| p Value | 0.046 | nd | nd | 0.42 | nd | 0.99 | <na | <0.091 | nd |
| 95% CI of | 1.0 | nd | nd | 0.089 | nd | 0.14 | >na | >0.74 | nd |
| OR Quart 2 | 68 | nd | nd | 2.7 | nd | 7.2 | na | na | nd |
| OR Quart 3 | 0 | nd | nd | 1.8 | nd | 2.0 | >6.3 | >0 | nd |
| p Value | na | nd | nd | 0.36 | nd | 0.42 | <0.091 | <na | nd |
| 95% CI of | na | nd | nd | 0.51 | nd | 0.36 | >0.74 | >na | nd |
| OR Quart 3 | na | nd | nd | 6.2 | nd | 11 | na | na | nd |
| OR Quart 4 | 1.0 | nd | nd | 0.49 | nd | 0.99 | >2.0 | >0 | nd |
| p Value | 1.0 | nd | nd | 0.41 | nd | 0.99 | <0.56 | <na | nd |
| 95% CI of | 0.062 | nd | nd | 0.088 | nd | 0.14 | >0.18 | >na | nd |
| OR Quart 4 | 16 | nd | nd | 2.7 | nd | 7.2 | na | na | nd |

TABLE 10
Comparison of marker levels in EDTA samples collected from
Cohort 1 (patients that did not progress beyond RIFLE stage 0, R, or I) and in EDTA
samples collected from Cohort 2 (subjects who progress to RIFLE stage F) at 0, 24 hours,
and 48 hours prior to the subject reaching RIFLE stage I.
C-C motif chemokine 18

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 182 | 272 | nd | nd |
| Average | nd | nd | 245 | 513 | nd | nd |
| Stdev | nd | nd | 205 | 604 | nd | nd |
| p (t-test) | nd | nd | | 0.0027 | nd | nd |
| Min | nd | nd | 31.7 | 92.4 | nd | nd |
| Max | nd | nd | 2110 | 1650 | nd | nd |
| n (Samp) | nd | nd | 367 | 6 | nd | nd |
| n (Patient) | nd | nd | 197 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.58 | nd | nd | nd | nd | nd |
| SE | nd | nd | nd | 0.12 | nd | nd | nd | nd | nd |
| p | nd | nd | nd | 0.51 | nd | nd | nd | nd | nd |
| nCohort 1 | nd | nd | nd | 367 | nd | nd | nd | nd | nd |
| nCohort 2 | nd | nd | nd | 6 | nd | nd | nd | nd | nd |
| Cutoff 1 | nd | nd | nd | 107 | nd | nd | nd | nd | nd |
| Sens 1 | nd | nd | nd | 83% | nd | nd | nd | nd | nd |
| Spec 1 | nd | nd | nd | 23% | nd | nd | nd | nd | nd |
| Cutoff 2 | nd | nd | nd | 107 | nd | nd | nd | nd | nd |
| Sens 2 | nd | nd | nd | 83% | nd | nd | nd | nd | nd |
| Spec 2 | nd | nd | nd | 23% | nd | nd | nd | nd | nd |
| Cutoff 3 | nd | nd | nd | 90.9 | nd | nd | nd | nd | nd |
| Sens 3 | nd | nd | nd | 100% | nd | nd | nd | nd | nd |
| Spec 3 | nd | nd | nd | 16% | nd | nd | nd | nd | nd |
| Cutoff 4 | nd | nd | nd | 278 | nd | nd | nd | nd | nd |
| Sens 4 | nd | nd | nd | 50% | nd | nd | nd | nd | nd |
| Spec 4 | nd | nd | nd | 70% | nd | nd | nd | nd | nd |
| Cutoff 5 | nd | nd | nd | 346 | nd | nd | nd | nd | nd |
| Sens 5 | nd | nd | nd | 50% | nd | nd | nd | nd | nd |
| Spec 5 | nd | nd | nd | 80% | nd | nd | nd | nd | nd |
| Cutoff 6 | nd | nd | nd | 491 | nd | nd | nd | nd | nd |
| Sens 6 | nd | nd | nd | 33% | nd | nd | nd | nd | nd |
| Spec 6 | nd | nd | nd | 90% | nd | nd | nd | nd | nd |
| OR Quart 2 | nd | nd | nd | 0.49 | nd | nd | nd | nd | nd |
| p Value | nd | nd | nd | 0.57 | nd | nd | nd | nd | nd |
| 95% CI of | nd | nd | nd | 0.044 | nd | nd | nd | nd | nd |
| OR Quart 2 | nd | nd | nd | 5.6 | nd | nd | nd | nd | nd |
| OR Quart 3 | nd | nd | nd | 0 | nd | nd | nd | nd | nd |
| p Value | nd | nd | nd | na | nd | nd | nd | nd | nd |
| 95% CI of | nd | nd | nd | na | nd | nd | nd | nd | nd |
| OR Quart 3 | nd | nd | nd | na | nd | nd | nd | nd | nd |
| OR Quart 4 | nd | nd | nd | 1.5 | nd | nd | nd | nd | nd |
| p Value | nd | nd | nd | 0.66 | nd | nd | nd | nd | nd |
| 95% CI of | nd | nd | nd | 0.24 | nd | nd | nd | nd | nd |
| OR Quart 4 | nd | nd | nd | 9.2 | nd | nd | nd | nd | nd |

C-C motif chemokine 24

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 254 | 278 | nd | nd |
| Average | nd | nd | 388 | 405 | nd | nd |
| Stdev | nd | nd | 385 | 395 | nd | nd |
| p (t-test) | nd | nd | | 0.92 | nd | nd |
| Min | nd | nd | 0.0260 | 81.9 | nd | nd |
| Max | nd | nd | 2920 | 1100 | nd | nd |
| n (Samp) | nd | nd | 297 | 6 | nd | nd |
| n (Patient) | nd | nd | 166 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.51 | nd | nd | nd | nd | nd |
| SE | nd | nd | nd | 0.12 | nd | nd | nd | nd | nd |
| p | nd | nd | nd | 0.93 | nd | nd | nd | nd | nd |
| nCohort 1 | nd | nd | nd | 297 | nd | nd | nd | nd | nd |
| nCohort 2 | nd | nd | nd | 6 | nd | nd | nd | nd | nd |
| Cutoff 1 | nd | nd | nd | 95.2 | nd | nd | nd | nd | nd |
| Sens 1 | nd | nd | nd | 83% | nd | nd | nd | nd | nd |
| Spec 1 | nd | nd | nd | 20% | nd | nd | nd | nd | nd |
| Cutoff 2 | nd | nd | nd | 95.2 | nd | nd | nd | nd | nd |
| Sens 2 | nd | nd | nd | 83% | nd | nd | nd | nd | nd |
| Spec 2 | nd | nd | nd | 20% | nd | nd | nd | nd | nd |
| Cutoff 3 | nd | nd | nd | 80.9 | nd | nd | nd | nd | nd |
| Sens 3 | nd | nd | nd | 100% | nd | nd | nd | nd | nd |
| Spec 3 | nd | nd | nd | 16% | nd | nd | nd | nd | nd |
| Cutoff 4 | nd | nd | nd | 475 | nd | nd | nd | nd | nd |
| Sens 4 | nd | nd | nd | 33% | nd | nd | nd | nd | nd |
| Spec 4 | nd | nd | nd | 70% | nd | nd | nd | nd | nd |
| Cutoff 5 | nd | nd | nd | 589 | nd | nd | nd | nd | nd |
| Sens 5 | nd | nd | nd | 33% | nd | nd | nd | nd | nd |
| Spec 5 | nd | nd | nd | 80% | nd | nd | nd | nd | nd |
| Cutoff 6 | nd | nd | nd | 900 | nd | nd | nd | nd | nd |
| Sens 6 | nd | nd | nd | 17% | nd | nd | nd | nd | nd |
| Spec 6 | nd | nd | nd | 90% | nd | nd | nd | nd | nd |
| OR Quart 2 | nd | nd | nd | 0.49 | nd | nd | nd | nd | nd |
| p Value | nd | nd | nd | 0.56 | nd | nd | nd | nd | nd |
| 95% CI of | nd | nd | nd | 0.043 | nd | nd | nd | nd | nd |
| OR Quart 2 | nd | nd | nd | 5.5 | nd | nd | nd | nd | nd |
| OR Quart 3 | nd | nd | nd | 0.49 | nd | nd | nd | nd | nd |
| p Value | nd | nd | nd | 0.56 | nd | nd | nd | nd | nd |
| 95% CI of | nd | nd | nd | 0.043 | nd | nd | nd | nd | nd |
| OR Quart 3 | nd | nd | nd | 5.5 | nd | nd | nd | nd | nd |
| OR Quart 4 | nd | nd | nd | 0.99 | nd | nd | nd | nd | nd |
| p Value | nd | nd | nd | 0.99 | nd | nd | nd | nd | nd |
| 95% CI of | nd | nd | nd | 0.14 | nd | nd | nd | nd | nd |
| OR Quart 4 | nd | nd | nd | 7.2 | nd | nd | nd | nd | nd |

C-C motif chemokine 8

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 13.4 | 16.7 | nd | nd |
| Average | nd | nd | 17.8 | 29.4 | nd | nd |
| Stdev | nd | nd | 23.5 | 35.7 | nd | nd |
| p (t-test) | nd | nd | | 0.24 | nd | nd |
| Min | nd | nd | 0.162 | 6.24 | nd | nd |
| Max | nd | nd | 180 | 101 | nd | nd |
| n (Samp) | nd | nd | 297 | 6 | nd | nd |
| n (Patient) | nd | nd | 166 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.62 | nd | nd | nd | nd | nd |
| SE | nd | nd | nd | 0.12 | nd | nd | nd | nd | nd |
| p | nd | nd | nd | 0.34 | nd | nd | nd | nd | nd |
| nCohort 1 | nd | nd | nd | 297 | nd | nd | nd | nd | nd |
| nCohort 2 | nd | nd | nd | 6 | nd | nd | nd | nd | nd |
| Cutoff 1 | nd | nd | nd | 11.5 | nd | nd | nd | nd | nd |
| Sens 1 | nd | nd | nd | 83% | nd | nd | nd | nd | nd |
| Spec 1 | nd | nd | nd | 39% | nd | nd | nd | nd | nd |
| Cutoff 2 | nd | nd | nd | 11.5 | nd | nd | nd | nd | nd |
| Sens 2 | nd | nd | nd | 83% | nd | nd | nd | nd | nd |
| Spec 2 | nd | nd | nd | 39% | nd | nd | nd | nd | nd |
| Cutoff 3 | nd | nd | nd | 5.84 | nd | nd | nd | nd | nd |
| Sens 3 | nd | nd | nd | 100% | nd | nd | nd | nd | nd |
| Spec 3 | nd | nd | nd | 17% | nd | nd | nd | nd | nd |
| Cutoff 4 | nd | nd | nd | 17.1 | nd | nd | nd | nd | nd |
| Sens 4 | nd | nd | nd | 50% | nd | nd | nd | nd | nd |
| Spec 4 | nd | nd | nd | 70% | nd | nd | nd | nd | nd |
| Cutoff 5 | nd | nd | nd | 20.1 | nd | nd | nd | nd | nd |
| Sens 5 | nd | nd | nd | 33% | nd | nd | nd | nd | nd |
| Spec 5 | nd | nd | nd | 80% | nd | nd | nd | nd | nd |
| Cutoff 6 | nd | nd | nd | 30.5 | nd | nd | nd | nd | nd |
| Sens 6 | nd | nd | nd | 17% | nd | nd | nd | nd | nd |
| Spec 6 | nd | nd | nd | 90% | nd | nd | nd | nd | nd |
| OR Quart 2 | nd | nd | nd | 0.99 | nd | nd | nd | nd | nd |
| p Value | nd | nd | nd | 0.99 | nd | nd | nd | nd | nd |
| 95% CI of | nd | nd | nd | 0.061 | nd | nd | nd | nd | nd |
| OR Quart 2 | nd | nd | nd | 16 | nd | nd | nd | nd | nd |
| OR Quart 3 | nd | nd | nd | 0.99 | nd | nd | nd | nd | nd |
| p Value | nd | nd | nd | 0.99 | nd | nd | nd | nd | nd |
| 95% CI of | nd | nd | nd | 0.061 | nd | nd | nd | nd | nd |
| OR Quart 3 | nd | nd | nd | 16 | nd | nd | nd | nd | nd |
| OR Quart 4 | nd | nd | nd | 3.0 | nd | nd | nd | nd | nd |
| p Value | nd | nd | nd | 0.34 | nd | nd | nd | nd | nd |
| 95% CI of | nd | nd | nd | 0.31 | nd | nd | nd | nd | nd |
| OR Quart 4 | nd | nd | nd | 30 | nd | nd | nd | nd | nd |

Cathepsin D

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 225000 | 379000 | nd | nd |
| Average | nd | nd | 260000 | 408000 | nd | nd |
| Stdev | nd | nd | 165000 | 159000 | nd | nd |
| p (t-test) | nd | nd | | 0.030 | nd | nd |
| Min | nd | nd | 34100 | 230000 | nd | nd |
| Max | nd | nd | 1870000 | 655000 | nd | nd |
| n (Samp) | nd | nd | 366 | 6 | nd | nd |
| n (Patient) | nd | nd | 196 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.79 | nd | nd | nd | nd | nd |
| SE | nd | nd | nd | 0.11 | nd | nd | nd | nd | nd |
| p | nd | nd | nd | 0.0096 | nd | nd | nd | nd | nd |
| nCohort 1 | nd | nd | nd | 366 | nd | nd | nd | nd | nd |
| nCohort 2 | nd | nd | nd | 6 | nd | nd | nd | nd | nd |
| Cutoff 1 | nd | nd | nd | 295000 | nd | nd | nd | nd | nd |
| Sens 1 | nd | nd | nd | 83% | nd | nd | nd | nd | nd |
| Spec 1 | nd | nd | nd | 67% | nd | nd | nd | nd | nd |
| Cutoff 2 | nd | nd | nd | 295000 | nd | nd | nd | nd | nd |
| Sens 2 | nd | nd | nd | 83% | nd | nd | nd | nd | nd |
| Spec 2 | nd | nd | nd | 67% | nd | nd | nd | nd | nd |
| Cutoff 3 | nd | nd | nd | 229000 | nd | nd | nd | nd | nd |
| Sens 3 | nd | nd | nd | 100% | nd | nd | nd | nd | nd |
| Spec 3 | nd | nd | nd | 51% | nd | nd | nd | nd | nd |
| Cutoff 4 | nd | nd | nd | 306000 | nd | nd | nd | nd | nd |
| Sens 4 | nd | nd | nd | 67% | nd | nd | nd | nd | nd |
| Spec 4 | nd | nd | nd | 70% | nd | nd | nd | nd | nd |
| Cutoff 5 | nd | nd | nd | 357000 | nd | nd | nd | nd | nd |
| Sens 5 | nd | nd | nd | 50% | nd | nd | nd | nd | nd |
| Spec 5 | nd | nd | nd | 80% | nd | nd | nd | nd | nd |
| Cutoff 6 | nd | nd | nd | 449000 | nd | nd | nd | nd | nd |
| Sens 6 | nd | nd | nd | 50% | nd | nd | nd | nd | nd |
| Spec 6 | nd | nd | nd | 90% | nd | nd | nd | nd | nd |
| OR Quart 2 | nd | nd | nd | >0 | nd | nd | nd | nd | nd |
| p Value | nd | nd | nd | <na | nd | nd | nd | nd | nd |
| 95% CI of | nd | nd | nd | >na | nd | nd | nd | nd | nd |
| OR Quart 2 | nd | nd | nd | na | nd | nd | nd | nd | nd |
| OR Quart 3 | nd | nd | nd | >3.1 | nd | nd | nd | nd | nd |
| p Value | nd | nd | nd | <0.33 | nd | nd | nd | nd | nd |
| 95% CI of | nd | nd | nd | >0.32 | nd | nd | nd | nd | nd |
| OR Quart 3 | nd | nd | nd | na | nd | nd | nd | nd | nd |
| OR Quart 4 | nd | nd | nd | >3.1 | nd | nd | nd | nd | nd |
| p Value | nd | nd | nd | <0.33 | nd | nd | nd | nd | nd |
| 95% CI of | nd | nd | nd | >0.32 | nd | nd | nd | nd | nd |
| OR Quart 4 | nd | nd | nd | na | nd | nd | nd | nd | nd |

C—X—C motif chemokine 13

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 51.9 | 73.9 | nd | nd |
| Average | nd | nd | 129 | 89.7 | nd | nd |
| Stdev | nd | nd | 258 | 39.2 | nd | nd |
| p (t-test) | nd | nd | | 0.71 | nd | nd |
| Min | nd | nd | 9.90 | 45.0 | nd | nd |
| Max | nd | nd | 2000 | 141 | nd | nd |
| n (Samp) | nd | nd | 297 | 6 | nd | nd |
| n (Patient) | nd | nd | 166 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.64 | nd | nd | nd | nd | nd |
| SE | nd | nd | nd | 0.12 | nd | nd | nd | nd | nd |
| p | nd | nd | nd | 0.24 | nd | nd | nd | nd | nd |
| nCohort 1 | nd | nd | nd | 297 | nd | nd | nd | nd | nd |
| nCohort 2 | nd | nd | nd | 6 | nd | nd | nd | nd | nd |
| Cutoff 1 | nd | nd | nd | 67.5 | nd | nd | nd | nd | nd |
| Sens 1 | nd | nd | nd | 83% | nd | nd | nd | nd | nd |
| Spec 1 | nd | nd | nd | 58% | nd | nd | nd | nd | nd |
| Cutoff 2 | nd | nd | nd | 67.5 | nd | nd | nd | nd | nd |
| Sens 2 | nd | nd | nd | 83% | nd | nd | nd | nd | nd |
| Spec 2 | nd | nd | nd | 58% | nd | nd | nd | nd | nd |
| Cutoff 3 | nd | nd | nd | 44.8 | nd | nd | nd | nd | nd |
| Sens 3 | nd | nd | nd | 100% | nd | nd | nd | nd | nd |
| Spec 3 | nd | nd | nd | 44% | nd | nd | nd | nd | nd |
| Cutoff 4 | nd | nd | nd | 95.3 | nd | nd | nd | nd | nd |
| Sens 4 | nd | nd | nd | 33% | nd | nd | nd | nd | nd |
| Spec 4 | nd | nd | nd | 70% | nd | nd | nd | nd | nd |
| Cutoff 5 | nd | nd | nd | 134 | nd | nd | nd | nd | nd |
| Sens 5 | nd | nd | nd | 33% | nd | nd | nd | nd | nd |
| Spec 5 | nd | nd | nd | 80% | nd | nd | nd | nd | nd |
| Cutoff 6 | nd | nd | nd | 250 | nd | nd | nd | nd | nd |
| Sens 6 | nd | nd | nd | 0% | nd | nd | nd | nd | nd |
| Spec 6 | nd | nd | nd | 90% | nd | nd | nd | nd | nd |
| OR Quart 2 | nd | nd | nd | >1.0 | nd | nd | nd | nd | nd |
| p Value | nd | nd | nd | <1.0 | nd | nd | nd | nd | nd |
| 95% CI of | nd | nd | nd | >0.061 | nd | nd | nd | nd | nd |
| OR Quart 2 | nd | nd | nd | na | nd | nd | nd | nd | nd |
| OR Quart 3 | nd | nd | nd | >3.1 | nd | nd | nd | nd | nd |
| p Value | nd | nd | nd | <0.33 | nd | nd | nd | nd | nd |
| 95% CI of | nd | nd | nd | >0.31 | nd | nd | nd | nd | nd |
| OR Quart 3 | nd | nd | nd | na | nd | nd | nd | nd | nd |
| OR Quart 4 | nd | nd | nd | >2.0 | nd | nd | nd | nd | nd |
| p Value | nd | nd | nd | <0.57 | nd | nd | nd | nd | nd |
| 95% CI of | nd | nd | nd | >0.18 | nd | nd | nd | nd | nd |
| OR Quart 4 | nd | nd | nd | na | nd | nd | nd | nd | nd |

Insulin-like growth factor-binding protein 3

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 3150 | 3200 | nd | nd |
| Average | nd | nd | 3500 | 3580 | nd | nd |
| Stdev | nd | nd | 1590 | 2040 | nd | nd |
| p (t-test) | nd | nd | | 0.90 | nd | nd |
| Min | nd | nd | 651 | 1380 | nd | nd |
| Max | nd | nd | 8600 | 7360 | nd | nd |
| n (Samp) | nd | nd | 367 | 6 | nd | nd |
| n (Patient) | nd | nd | 197 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.50 | nd | nd | nd | nd | nd |
| SE | nd | nd | nd | 0.12 | nd | nd | nd | nd | nd |
| p | nd | nd | nd | 0.98 | nd | nd | nd | nd | nd |
| nCohort 1 | nd | nd | nd | 367 | nd | nd | nd | nd | nd |
| nCohort 2 | nd | nd | nd | 6 | nd | nd | nd | nd | nd |
| Cutoff 1 | nd | nd | nd | 2410 | nd | nd | nd | nd | nd |
| Sens 1 | nd | nd | nd | 83% | nd | nd | nd | nd | nd |
| Spec 1 | nd | nd | nd | 30% | nd | nd | nd | nd | nd |
| Cutoff 2 | nd | nd | nd | 2410 | nd | nd | nd | nd | nd |
| Sens 2 | nd | nd | nd | 83% | nd | nd | nd | nd | nd |
| Spec 2 | nd | nd | nd | 30% | nd | nd | nd | nd | nd |
| Cutoff 3 | nd | nd | nd | 1380 | nd | nd | nd | nd | nd |
| Sens 3 | nd | nd | nd | 100% | nd | nd | nd | nd | nd |
| Spec 3 | nd | nd | nd | 6% | nd | nd | nd | nd | nd |
| Cutoff 4 | nd | nd | nd | 4250 | nd | nd | nd | nd | nd |
| Sens 4 | nd | nd | nd | 17% | nd | nd | nd | nd | nd |
| Spec 4 | nd | nd | nd | 70% | nd | nd | nd | nd | nd |
| Cutoff 5 | nd | nd | nd | 4870 | nd | nd | nd | nd | nd |
| Sens 5 | nd | nd | nd | 17% | nd | nd | nd | nd | nd |
| Spec 5 | nd | nd | nd | 80% | nd | nd | nd | nd | nd |
| Cutoff 6 | nd | nd | nd | 5780 | nd | nd | nd | nd | nd |
| Sens 6 | nd | nd | nd | 17% | nd | nd | nd | nd | nd |
| Spec 6 | nd | nd | nd | 90% | nd | nd | nd | nd | nd |
| OR Quart 2 | nd | nd | nd | 2.0 | nd | nd | nd | nd | nd |
| p Value | nd | nd | nd | 0.56 | nd | nd | nd | nd | nd |
| 95% CI of | nd | nd | nd | 0.18 | nd | nd | nd | nd | nd |
| OR Quart 2 | nd | nd | nd | 23 | nd | nd | nd | nd | nd |
| OR Quart 3 | nd | nd | nd | 2.0 | nd | nd | nd | nd | nd |
| p Value | nd | nd | nd | 0.56 | nd | nd | nd | nd | nd |
| 95% CI of | nd | nd | nd | 0.18 | nd | nd | nd | nd | nd |
| OR Quart 3 | nd | nd | nd | 23 | nd | nd | nd | nd | nd |
| OR Quart 4 | nd | nd | nd | 1.0 | nd | nd | nd | nd | nd |
| p Value | nd | nd | nd | 0.99 | nd | nd | nd | nd | nd |
| 95% CI of | nd | nd | nd | 0.062 | nd | nd | nd | nd | nd |
| OR Quart 4 | nd | nd | nd | 16 | nd | nd | nd | nd | nd |

Interleukin-11

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 62.8 | 83.7 | nd | nd |
| Average | nd | nd | 147 | 102 | nd | nd |
| Stdev | nd | nd | 555 | 53.4 | nd | nd |
| p (t-test) | nd | nd | | 0.84 | nd | nd |
| Min | nd | nd | 0.359 | 51.1 | nd | nd |
| Max | nd | nd | 6920 | 171 | nd | nd |
| n (Samp) | nd | nd | 298 | 6 | nd | nd |
| n (Patient) | nd | nd | 167 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.63 | nd | nd | nd | nd | nd |
| SE | nd | nd | nd | 0.12 | nd | nd | nd | nd | nd |
| p | nd | nd | nd | 0.29 | nd | nd | nd | nd | nd |
| nCohort 1 | nd | nd | nd | 298 | nd | nd | nd | nd | nd |
| nCohort 2 | nd | nd | nd | 6 | nd | nd | nd | nd | nd |
| Cutoff 1 | nd | nd | nd | 51.1 | nd | nd | nd | nd | nd |
| Sens 1 | nd | nd | nd | 83% | nd | nd | nd | nd | nd |
| Spec 1 | nd | nd | nd | 46% | nd | nd | nd | nd | nd |
| Cutoff 2 | nd | nd | nd | 51.1 | nd | nd | nd | nd | nd |
| Sens 2 | nd | nd | nd | 83% | nd | nd | nd | nd | nd |
| Spec 2 | nd | nd | nd | 46% | nd | nd | nd | nd | nd |
| Cutoff 3 | nd | nd | nd | 47.4 | nd | nd | nd | nd | nd |
| Sens 3 | nd | nd | nd | 100% | nd | nd | nd | nd | nd |
| Spec 3 | nd | nd | nd | 46% | nd | nd | nd | nd | nd |
| Cutoff 4 | nd | nd | nd | 111 | nd | nd | nd | nd | nd |
| Sens 4 | nd | nd | nd | 33% | nd | nd | nd | nd | nd |
| Spec 4 | nd | nd | nd | 70% | nd | nd | nd | nd | nd |
| Cutoff 5 | nd | nd | nd | 158 | nd | nd | nd | nd | nd |
| Sens 5 | nd | nd | nd | 33% | nd | nd | nd | nd | nd |
| Spec 5 | nd | nd | nd | 81% | nd | nd | nd | nd | nd |
| Cutoff 6 | nd | nd | nd | 221 | nd | nd | nd | nd | nd |
| Sens 6 | nd | nd | nd | 0% | nd | nd | nd | nd | nd |
| Spec 6 | nd | nd | nd | 90% | nd | nd | nd | nd | nd |
| OR Quart 2 | nd | nd | nd | >2.1 | nd | nd | nd | nd | nd |
| p Value | nd | nd | nd | <0.56 | nd | nd | nd | nd | nd |
| 95% CI of | nd | nd | nd | >0.18 | nd | nd | nd | nd | nd |
| OR Quart 2 | nd | nd | nd | na | nd | nd | nd | nd | nd |
| OR Quart 3 | nd | nd | nd | >2.1 | nd | nd | nd | nd | nd |
| p Value | nd | nd | nd | <0.56 | nd | nd | nd | nd | nd |
| 95% CI of | nd | nd | nd | >0.18 | nd | nd | nd | nd | nd |
| OR Quart 3 | nd | nd | nd | na | nd | nd | nd | nd | nd |
| OR Quart 4 | nd | nd | nd | >2.1 | nd | nd | nd | nd | nd |
| p Value | nd | nd | nd | <0.56 | nd | nd | nd | nd | nd |
| 95% CI of | nd | nd | nd | >0.18 | nd | nd | nd | nd | nd |
| OR Quart 4 | nd | nd | nd | na | nd | nd | nd | nd | nd |

Interleukin-2 receptor alpha chain

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 19.4 | 28.9 | nd | nd |
| Average | nd | nd | 70.5 | 48.1 | nd | nd |
| Stdev | nd | nd | 148 | 56.2 | nd | nd |
| p (t-test) | nd | nd | | 0.71 | nd | nd |
| Min | nd | nd | 0.0290 | 0.0290 | nd | nd |
| Max | nd | nd | 1040 | 154 | nd | nd |
| n (Samp) | nd | nd | 298 | 6 | nd | nd |
| n (Patient) | nd | nd | 167 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.54 | nd | nd | nd | nd | nd |
| SE | nd | nd | nd | 0.12 | nd | nd | nd | nd | nd |
| p | nd | nd | nd | 0.77 | nd | nd | nd | nd | nd |
| nCohort 1 | nd | nd | nd | 298 | nd | nd | nd | nd | nd |
| nCohort 2 | nd | nd | nd | 6 | nd | nd | nd | nd | nd |
| Cutoff 1 | nd | nd | nd | 11.9 | nd | nd | nd | nd | nd |
| Sens 1 | nd | nd | nd | 83% | nd | nd | nd | nd | nd |
| Spec 1 | nd | nd | nd | 43% | nd | nd | nd | nd | nd |
| Cutoff 2 | nd | nd | nd | 11.9 | nd | nd | nd | nd | nd |
| Sens 2 | nd | nd | nd | 83% | nd | nd | nd | nd | nd |
| Spec 2 | nd | nd | nd | 43% | nd | nd | nd | nd | nd |
| Cutoff 3 | nd | nd | nd | 0 | nd | nd | nd | nd | nd |
| Sens 3 | nd | nd | nd | 100% | nd | nd | nd | nd | nd |
| Spec 3 | nd | nd | nd | 0% | nd | nd | nd | nd | nd |
| Cutoff 4 | nd | nd | nd | 50.7 | nd | nd | nd | nd | nd |
| Sens 4 | nd | nd | nd | 33% | nd | nd | nd | nd | nd |
| Spec 4 | nd | nd | nd | 70% | nd | nd | nd | nd | nd |
| Cutoff 5 | nd | nd | nd | 92.7 | nd | nd | nd | nd | nd |
| Sens 5 | nd | nd | nd | 17% | nd | nd | nd | nd | nd |
| Spec 5 | nd | nd | nd | 80% | nd | nd | nd | nd | nd |
| Cutoff 6 | nd | nd | nd | 167 | nd | nd | nd | nd | nd |
| Sens 6 | nd | nd | nd | 0% | nd | nd | nd | nd | nd |
| Spec 6 | nd | nd | nd | 90% | nd | nd | nd | nd | nd |
| OR Quart 2 | nd | nd | nd | 1.0 | nd | nd | nd | nd | nd |
| p Value | nd | nd | nd | 1.0 | nd | nd | nd | nd | nd |
| 95% CI of | nd | nd | nd | 0.061 | nd | nd | nd | nd | nd |
| OR Quart 2 | nd | nd | nd | 16 | nd | nd | nd | nd | nd |
| OR Quart 3 | nd | nd | nd | 3.1 | nd | nd | nd | nd | nd |
| p Value | nd | nd | nd | 0.33 | nd | nd | nd | nd | nd |
| 95% CI of | nd | nd | nd | 0.31 | nd | nd | nd | nd | nd |
| OR Quart 3 | nd | nd | nd | 30 | nd | nd | nd | nd | nd |
| OR Quart 4 | nd | nd | nd | 1.0 | nd | nd | nd | nd | nd |
| p Value | nd | nd | nd | 1.0 | nd | nd | nd | nd | nd |
| 95% CI of | nd | nd | nd | 0.061 | nd | nd | nd | nd | nd |
| OR Quart 4 | nd | nd | nd | 16 | nd | nd | nd | nd | nd |

Neutrophil collagenase

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 1100 | 5350 | nd | nd |
| Average | nd | nd | 3160 | 14200 | nd | nd |
| Stdev | nd | nd | 6940 | 17400 | nd | nd |
| p (t-test) | nd | nd | | 2.3E−4 | nd | nd |
| Min | nd | nd | 40.0 | 1.14 | nd | nd |
| Max | nd | nd | 55000 | 38700 | nd | nd |
| n (Samp) | nd | nd | 366 | 6 | nd | nd |
| n (Patient) | nd | nd | 197 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.72 | nd | nd | nd | nd | nd |
| SE | nd | nd | nd | 0.12 | nd | nd | nd | nd | nd |
| p | nd | nd | nd | 0.059 | nd | nd | nd | nd | nd |
| nCohort 1 | nd | nd | nd | 366 | nd | nd | nd | nd | nd |
| nCohort 2 | nd | nd | nd | 6 | nd | nd | nd | nd | nd |
| Cutoff 1 | nd | nd | nd | 1500 | nd | nd | nd | nd | nd |
| Sens 1 | nd | nd | nd | 83% | nd | nd | nd | nd | nd |
| Spec 1 | nd | nd | nd | 64% | nd | nd | nd | nd | nd |
| Cutoff 2 | nd | nd | nd | 1500 | nd | nd | nd | nd | nd |
| Sens 2 | nd | nd | nd | 83% | nd | nd | nd | nd | nd |
| Spec 2 | nd | nd | nd | 64% | nd | nd | nd | nd | nd |
| Cutoff 3 | nd | nd | nd | 0 | nd | nd | nd | nd | nd |
| Sens 3 | nd | nd | nd | 100% | nd | nd | nd | nd | nd |
| Spec 3 | nd | nd | nd | 0% | nd | nd | nd | nd | nd |
| Cutoff 4 | nd | nd | nd | 1830 | nd | nd | nd | nd | nd |
| Sens 4 | nd | nd | nd | 67% | nd | nd | nd | nd | nd |
| Spec 4 | nd | nd | nd | 70% | nd | nd | nd | nd | nd |
| Cutoff 5 | nd | nd | nd | 3110 | nd | nd | nd | nd | nd |
| Sens 5 | nd | nd | nd | 67% | nd | nd | nd | nd | nd |
| Spec 5 | nd | nd | nd | 80% | nd | nd | nd | nd | nd |
| Cutoff 6 | nd | nd | nd | 6510 | nd | nd | nd | nd | nd |
| Sens 6 | nd | nd | nd | 50% | nd | nd | nd | nd | nd |
| Spec 6 | nd | nd | nd | 90% | nd | nd | nd | nd | nd |
| OR Quart 2 | nd | nd | nd | 0 | nd | nd | nd | nd | nd |
| p Value | nd | nd | nd | na | nd | nd | nd | nd | nd |
| 95% CI of | nd | nd | nd | na | nd | nd | nd | nd | nd |
| OR Quart 2 | nd | nd | nd | na | nd | nd | nd | nd | nd |
| OR Quart 3 | nd | nd | nd | 1.0 | nd | nd | nd | nd | nd |
| p Value | nd | nd | nd | 1.0 | nd | nd | nd | nd | nd |
| 95% CI of | nd | nd | nd | 0.062 | nd | nd | nd | nd | nd |
| OR Quart 3 | nd | nd | nd | 16 | nd | nd | nd | nd | nd |
| OR Quart 4 | nd | nd | nd | 4.1 | nd | nd | nd | nd | nd |
| p Value | nd | nd | nd | 0.21 | nd | nd | nd | nd | nd |
| 95% CI of | nd | nd | nd | 0.45 | nd | nd | nd | nd | nd |
| OR Quart 4 | nd | nd | nd | 38 | nd | nd | nd | nd | nd |

Protransforming growth factor alpha

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 1.45 | 6.40 | nd | nd |
| Average | nd | nd | 7.21 | 17.3 | nd | nd |
| Stdev | nd | nd | 27.5 | 26.5 | nd | nd |
| p (t-test) | nd | nd | | 0.37 | nd | nd |
| Min | nd | nd | 0.00228 | 0.0105 | nd | nd |
| Max | nd | nd | 287 | 68.3 | nd | nd |
| n (Samp) | nd | nd | 298 | 6 | nd | nd |
| n (Patient) | nd | nd | 167 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.60 | nd | nd | nd | nd | nd |
| SE | nd | nd | nd | 0.12 | nd | nd | nd | nd | nd |
| p | nd | nd | nd | 0.42 | nd | nd | nd | nd | nd |
| nCohort 1 | nd | nd | nd | 298 | nd | nd | nd | nd | nd |
| nCohort 2 | nd | nd | nd | 6 | nd | nd | nd | nd | nd |
| Cutoff 1 | nd | nd | nd | 0.190 | nd | nd | nd | nd | nd |
| Sens 1 | nd | nd | nd | 83% | nd | nd | nd | nd | nd |
| Spec 1 | nd | nd | nd | 23% | nd | nd | nd | nd | nd |
| Cutoff 2 | nd | nd | nd | 0.190 | nd | nd | nd | nd | nd |
| Sens 2 | nd | nd | nd | 83% | nd | nd | nd | nd | nd |
| Spec 2 | nd | nd | nd | 23% | nd | nd | nd | nd | nd |
| Cutoff 3 | nd | nd | nd | 0.00584 | nd | nd | nd | nd | nd |
| Sens 3 | nd | nd | nd | 100% | nd | nd | nd | nd | nd |
| Spec 3 | nd | nd | nd | 16% | nd | nd | nd | nd | nd |
| Cutoff 4 | nd | nd | nd | 3.12 | nd | nd | nd | nd | nd |
| Sens 4 | nd | nd | nd | 50% | nd | nd | nd | nd | nd |
| Spec 4 | nd | nd | nd | 70% | nd | nd | nd | nd | nd |
| Cutoff 5 | nd | nd | nd | 4.80 | nd | nd | nd | nd | nd |
| Sens 5 | nd | nd | nd | 50% | nd | nd | nd | nd | nd |
| Spec 5 | nd | nd | nd | 81% | nd | nd | nd | nd | nd |
| Cutoff 6 | nd | nd | nd | 9.07 | nd | nd | nd | nd | nd |
| Sens 6 | nd | nd | nd | 50% | nd | nd | nd | nd | nd |
| Spec 6 | nd | nd | nd | 90% | nd | nd | nd | nd | nd |
| OR Quart 2 | nd | nd | nd | 0.49 | nd | nd | nd | nd | nd |
| p Value | nd | nd | nd | 0.57 | nd | nd | nd | nd | nd |
| 95% CI of | nd | nd | nd | 0.044 | nd | nd | nd | nd | nd |
| OR Quart 2 | nd | nd | nd | 5.6 | nd | nd | nd | nd | nd |
| OR Quart 3 | nd | nd | nd | 0 | nd | nd | nd | nd | nd |
| p Value | nd | nd | nd | na | nd | nd | nd | nd | nd |
| 95% CI of | nd | nd | nd | na | nd | nd | nd | nd | nd |
| OR Quart 3 | nd | nd | nd | na | nd | nd | nd | nd | nd |
| OR Quart 4 | nd | nd | nd | 1.5 | nd | nd | nd | nd | nd |
| p Value | nd | nd | nd | 0.65 | nd | nd | nd | nd | nd |
| 95% CI of | nd | nd | nd | 0.25 | nd | nd | nd | nd | nd |
| OR Quart 4 | nd | nd | nd | 9.4 | nd | nd | nd | nd | nd |

CA 15-3

| | | |
|---|---|---|
| 0 hr prior to | 24 hr prior to | 48 hr prior to |
| AKI stage | AKI stage | AKI stage |

| sCr or UO | Cohort 1 | Cohort 2 | Cohort 1 | Cohort 2 | Cohort 1 | Cohort 2 |
|---|---|---|---|---|---|---|
| Median | nd | nd | 0.286 | 0.833 | nd | nd |
| Average | nd | nd | 1.00 | 1.13 | nd | nd |
| Stdev | nd | nd | 4.15 | 0.884 | nd | nd |
| p (t-test) | nd | nd | | 0.94 | nd | nd |
| Min | nd | nd | 0.00154 | 0.249 | nd | nd |
| Max | nd | nd | 60.0 | 2.69 | nd | nd |
| n (Samp) | nd | nd | 367 | 6 | nd | nd |
| n (Patient) | nd | nd | 197 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.80 | nd | nd | nd | nd | nd |
| SE | nd | nd | nd | 0.11 | nd | nd | nd | nd | nd |
| p | nd | nd | nd | 0.0065 | nd | nd | nd | nd | nd |
| nCohort 1 | nd | nd | nd | 367 | nd | nd | nd | nd | nd |
| nCohort 2 | nd | nd | nd | 6 | nd | nd | nd | nd | nd |
| Cutoff 1 | nd | nd | nd | 0.597 | nd | nd | nd | nd | nd |
| Sens 1 | nd | nd | nd | 83% | nd | nd | nd | nd | nd |
| Spec 1 | nd | nd | nd | 78% | nd | nd | nd | nd | nd |
| Cutoff 2 | nd | nd | nd | 0.597 | nd | nd | nd | nd | nd |
| Sens 2 | nd | nd | nd | 83% | nd | nd | nd | nd | nd |
| Spec 2 | nd | nd | nd | 78% | nd | nd | nd | nd | nd |
| Cutoff 3 | nd | nd | nd | 0.249 | nd | nd | nd | nd | nd |
| Sens 3 | nd | nd | nd | 100% | nd | nd | nd | nd | nd |
| Spec 3 | nd | nd | nd | 46% | nd | nd | nd | nd | nd |
| Cutoff 4 | nd | nd | nd | 0.466 | nd | nd | nd | nd | nd |
| Sens 4 | nd | nd | nd | 83% | nd | nd | nd | nd | nd |
| Spec 4 | nd | nd | nd | 70% | nd | nd | nd | nd | nd |
| Cutoff 5 | nd | nd | nd | 0.680 | nd | nd | nd | nd | nd |
| Sens 5 | nd | nd | nd | 67% | nd | nd | nd | nd | nd |
| Spec 5 | nd | nd | nd | 80% | nd | nd | nd | nd | nd |
| Cutoff 6 | nd | nd | nd | 1.26 | nd | nd | nd | nd | nd |
| Sens 6 | nd | nd | nd | 33% | nd | nd | nd | nd | nd |
| Spec 6 | nd | nd | nd | 90% | nd | nd | nd | nd | nd |
| OR Quart 2 | nd | nd | nd | >1.0 | nd | nd | nd | nd | nd |
| p Value | nd | nd | nd | <0.99 | nd | nd | nd | nd | nd |
| 95% CI of | nd | nd | nd | >0.062 | nd | nd | nd | nd | nd |
| OR Quart 2 | nd | nd | nd | na | nd | nd | nd | nd | nd |
| OR Quart 3 | nd | nd | nd | >0 | nd | nd | nd | nd | nd |
| p Value | nd | nd | nd | <na | nd | nd | nd | nd | nd |
| 95% CI of | nd | nd | nd | >na | nd | nd | nd | nd | nd |
| OR Quart 3 | nd | nd | nd | na | nd | nd | nd | nd | nd |
| OR Quart 4 | nd | nd | nd | >5.2 | nd | nd | nd | nd | nd |
| p Value | nd | nd | nd | <0.13 | nd | nd | nd | nd | nd |
| 95% CI of | nd | nd | nd | >0.60 | nd | nd | nd | nd | nd |
| OR Quart 4 | nd | nd | nd | na | nd | nd | nd | nd | nd |

TABLE 11
Comparison of marker levels in enroll urine samples collected from
Cohort 1 (patients that did not progress beyond RIFLE stage 0 or R
within 48 hrs) and in enroll urine samples collected from Cohort 2
(subjects reaching RIFLE stage I or F within 48 hrs). Enroll samples
from patients already at RIFLE stage I or F were included in Cohort 2.
C-C motif chemokine 18

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 0.460 | 1.04 | 0.486 | 2.03 | 0.488 | 1.04 |
| Average | 1.95 | 6.61 | 2.58 | 9.28 | 2.03 | 7.35 |
| Stdev | 5.90 | 12.7 | 7.41 | 14.4 | 5.85 | 13.5 |
| p (t-test) | | 2.7E−7 | | 2.0E−4 | | 3.3E−7 |
| Min | 3.13E−5 | 0.00268 | 3.13E−5 | 0.204 | 3.13E−5 | 0.00268 |
| Max | 40.0 | 40.0 | 40.0 | 40.0 | 40.0 | 40.0 |
| n (Samp) | 383 | 92 | 451 | 20 | 296 | 79 |
| n (Patient) | 383.00 | 92 | 451 | 20 | 296 | 79 |

| | At Enrollment | | |
|---|---|---|---|
| | sCr or UO | sCr only | UO only |
| AUC | 0.66 | 0.75 | 0.64 |
| SE | 0.034 | 0.064 | 0.037 |
| p | 2.3E−6 | 9.2E−5 | 1.4E−4 |
| nCohort 1 | 383 | 451 | 296 |
| nCohort 2 | 92 | 20 | 79 |
| Cutoff 1 | 0.402 | 0.906 | 0.362 |
| Sens 1 | 71% | 70% | 71% |
| Spec 1 | 48% | 67% | 43% |
| Cutoff 2 | 0.274 | 0.826 | 0.264 |
| Sens 2 | 80% | 80% | 81% |
| Spec 2 | 39% | 63% | 33% |
| Cutoff 3 | 0.147 | 0.340 | 0.141 |
| Sens 3 | 90% | 90% | 91% |
| Spec 3 | 24% | 42% | 19% |
| Cutoff 4 | 0.928 | 1.10 | 1.01 |
| Sens 4 | 53% | 60% | 52% |
| Spec 4 | 70% | 70% | 70% |
| Cutoff 5 | 1.61 | 1.84 | 1.81 |
| Sens 5 | 41% | 50% | 39% |
| Spec 5 | 80% | 80% | 80% |
| Cutoff 6 | 3.46 | 3.81 | 3.59 |
| Sens 6 | 26% | 35% | 25% |
| Spec 6 | 90% | 90% | 90% |
| OR Quart 2 | 2.3 | >3.1 | 1.7 |
| p Value | 0.031 | <0.34 | 0.18 |
| 95% CI of | 1.1 | >0.31 | 0.78 |
| OR Quart 2 | 5.0 | na | 3.8 |
| OR Quart 3 | 1.8 | >6.3 | 1.3 |
| p Value | 0.13 | <0.092 | 0.55 |
| 95% CI of | 0.84 | >0.74 | 0.56 |
| OR Quart 3 | 4.1 | na | 2.9 |
| OR Quart 4 | 4.7 | >12 | 3.7 |
| p Value | 2.9E−5 | <0.018 | 6.0E−4 |
| 95% CI of | 2.3 | >1.5 | 1.7 |
| OR Quart 4 | 9.8 | na | 7.7 |

C-C motif chemokine 24

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 10.6 | 36.2 | 13.9 | 79.4 | 11.4 | 36.1 |
| Average | 24.0 | 134 | 39.6 | 172 | 24.8 | 144 |
| Stdev | 47.0 | 357 | 147 | 418 | 49.4 | 384 |
| p (t-test) | | 1.1E−8 | | 5.4E−4 | | 3.3E−7 |
| Min | 0.0120 | 0.0347 | 0.0120 | 3.66 | 0.0120 | 0.0347 |
| Max | 554 | 2380 | 2380 | 1930 | 554 | 2380 |
| n (Samp) | 383 | 92 | 451 | 20 | 297 | 79 |
| n (Patient) | 383 | 92 | 451 | 20 | 297 | 79 |

| | At Enrollment | | |
|---|---|---|---|
| | sCr or UO | sCr only | UO only |
| AUC | 0.76 | 0.81 | 0.75 |
| SE | 0.031 | 0.059 | 0.034 |
| p | 0 | 1.4E−7 | 2.5E−13 |
| nCohort 1 | 383 | 451 | 297 |
| nCohort 2 | 92 | 20 | 79 |
| Cutoff 1 | 24.0 | 27.3 | 21.2 |
| Sens 1 | 71% | 70% | 71% |
| Spec 1 | 72% | 71% | 69% |
| Cutoff 2 | 14.5 | 27.0 | 11.7 |
| Sens 2 | 80% | 80% | 81% |
| Spec 2 | 58% | 71% | 51% |
| Cutoff 3 | 7.72 | 20.4 | 5.54 |
| Sens 3 | 90% | 90% | 91% |
| Spec 3 | 42% | 61% | 35% |
| Cutoff 4 | 22.4 | 26.6 | 22.4 |
| Sens 4 | 71% | 80% | 70% |
| Spec 4 | 70% | 70% | 70% |
| Cutoff 5 | 31.0 | 38.5 | 29.7 |
| Sens 5 | 54% | 60% | 57% |
| Spec 5 | 80% | 80% | 80% |
| Cutoff 6 | 57.3 | 67.2 | 57.9 |
| Sens 6 | 37% | 55% | 35% |
| Spec 6 | 90% | 90% | 90% |
| OR Quart 2 | 2.8 | >2.0 | 2.6 |
| p Value | 0.061 | <0.57 | 0.084 |
| 95% CI of | 0.96 | >0.18 | 0.88 |
| OR Quart 2 | 8.0 | na | 7.7 |
| OR Quart 3 | 6.6 | >6.3 | 5.4 |
| p Value | 1.9E−4 | <0.092 | 0.0011 |
| 95% CI of | 2.5 | >0.74 | 2.0 |
| OR Quart 3 | 18 | na | 15 |
| OR Quart 4 | 15 | >13 | 13 |
| p Value | 5.1E−8 | <0.014 | 3.2E−7 |
| 95% CI of | 5.6 | >1.7 | 4.9 |
| OR Quart 4 | 39 | na | 35 |

C-C motif chemokine 8

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 1.36 | 6.27 | 1.51 | 19.7 | 1.55 | 6.84 |
| Average | 11.6 | 106 | 14.8 | 376 | 13.1 | 74.4 |
| Stdev | 35.6 | 494 | 45.8 | 1020 | 39.9 | 345 |
| p (t-test) | | 2.2E−4 | | 3.3E−13 | | 0.0029 |
| Min | 0.0250 | 0.0250 | 0.0250 | 0.0701 | 0.0250 | 0.0250 |
| Max | 473 | 3670 | 492 | 3670 | 473 | 3020 |
| n (Samp) | 383 | 92 | 451 | 20 | 297 | 79 |
| n (Patient) | 383 | 92 | 451 | 20 | 297 | 79 |

| | At Enrollment | | |
|---|---|---|---|
| | sCr or UO | sCr only | UO only |
| AUC | 0.64 | 0.70 | 0.63 |
| SE | 0.034 | 0.067 | 0.037 |
| p | 3.2E−5 | 0.0024 | 3.7E−4 |
| nCohort 1 | 383 | 451 | 297 |
| nCohort 2 | 92 | 20 | 79 |
| Cutoff 1 | 0.491 | 3.16 | 0.491 |
| Sens 1 | 71% | 70% | 71% |
| Spec 1 | 38% | 58% | 35% |
| Cutoff 2 | 0.240 | 0.491 | 0.240 |
| Sens 2 | 80% | 80% | 82% |
| Spec 2 | 35% | 37% | 31% |
| Cutoff 3 | 0.127 | 0.154 | 0.127 |
| Sens 3 | 91% | 90% | 91% |
| Spec 3 | 27% | 28% | 23% |
| Cutoff 4 | 7.03 | 7.99 | 7.18 |
| Sens 4 | 48% | 60% | 49% |
| Spec 4 | 70% | 70% | 70% |
| Cutoff 5 | 12.0 | 12.7 | 12.2 |
| Sens 5 | 36% | 55% | 34% |
| Spec 5 | 80% | 80% | 80% |
| Cutoff 6 | 24.5 | 30.8 | 24.5 |
| Sens 6 | 27% | 35% | 28% |
| Spec 6 | 90% | 90% | 91% |
| OR Quart 2 | 3.4 | 5.1 | 1.8 |
| p Value | 0.0031 | 0.14 | 0.16 |
| 95% CI of | 1.5 | 0.59 | 0.79 |
| OR Quart 2 | 7.6 | 45 | 4.0 |
| OR Quart 3 | 2.4 | 2.0 | 2.2 |
| p Value | 0.035 | 0.57 | 0.056 |
| 95% CI of | 1.1 | 0.18 | 0.98 |
| OR Quart 3 | 5.6 | 22 | 4.8 |
| OR Quart 4 | 5.5 | 13 | 3.4 |
| p Value | 2.1E−5 | 0.014 | 0.0019 |
| 95% CI of | 2.5 | 1.7 | 1.6 |
| OR Quart 4 | 12 | 100 | 7.2 |

Cathepsin D

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 72700 | 105000 | 75800 | 130000 | 75500 | 104000 |
| Average | 76500 | 111000 | 81000 | 130000 | 80300 | 106000 |
| Stdev | 40100 | 42500 | 41700 | 40400 | 39500 | 41400 |
| p (t-test) | | 2.0E−12 | | 4.8E−7 | | 3.7E−7 |
| Min | 656 | 4320 | 656 | 55700 | 2520 | 4320 |
| Max | 200000 | 200000 | 200000 | 200000 | 200000 | 200000 |
| n (Samp) | 383 | 92 | 451 | 20 | 296 | 79 |
| n (Patient) | 383 | 92 | 451 | 20 | 296 | 79 |

| | At Enrollment | | |
|---|---|---|---|
| | sCr or UO | sCr only | UO only |
| AUC | 0.72 | 0.80 | 0.68 |
| SE | 0.032 | 0.060 | 0.036 |
| p | 2.7E−12 | 6.5E−7 | 4.3E−7 |
| nCohort 1 | 383 | 451 | 296 |
| nCohort 2 | 92 | 20 | 79 |
| Cutoff 1 | 84100 | 108000 | 78200 |
| Sens 1 | 71% | 70% | 71% |
| Spec 1 | 63% | 76% | 53% |
| Cutoff 2 | 71300 | 99200 | 70400 |
| Sens 2 | 80% | 80% | 81% |
| Spec 2 | 49% | 69% | 46% |
| Cutoff 3 | 63600 | 84400 | 62300 |
| Sens 3 | 90% | 90% | 91% |
| Spec 3 | 42% | 59% | 37% |
| Cutoff 4 | 95800 | 102000 | 99200 |
| Sens 4 | 60% | 70% | 54% |
| Spec 4 | 70% | 70% | 70% |
| Cutoff 5 | 109000 | 116000 | 115000 |
| Sens 5 | 47% | 65% | 39% |
| Spec 5 | 80% | 80% | 80% |
| Cutoff 6 | 133000 | 138000 | 134000 |
| Sens 6 | 27% | 35% | 24% |
| Spec 6 | 90% | 90% | 90% |
| OR Quart 2 | 5.4 | >2.0 | 4.8 |
| p Value | 0.0029 | <0.57 | 0.0029 |
| 95% CI of | 1.8 | >0.18 | 1.7 |
| OR Quart 2 | 16 | na | 13 |
| OR Quart 3 | 8.0 | >5.2 | 5.1 |
| p Value | 1.8E−4 | <0.14 | 0.0019 |
| 95% CI of | 2.7 | >0.60 | 1.8 |
| OR Quart 3 | 24 | na | 14 |
| OR Quart 4 | 16 | >14 | 9.5 |
| p Value | 3.1E−7 | <0.011 | 9.2E−6 |
| 95% CI of | 5.6 | >1.9 | 3.5 |
| OR Quart 4 | 47 | na | 26 |

C—X—C motif chemokine 13

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 0.0733 | 2.45 | 0.236 | 7.12 | 0.156 | 2.43 |
| Average | 6.47 | 46.6 | 9.11 | 131 | 7.64 | 51.3 |
| Stdev | 37.8 | 201 | 42.6 | 413 | 42.7 | 216 |
| p (t-test) | | 2.8E−4 | | 1.8E−8 | | 0.0012 |
| Min | 0.00269 | 0.00269 | 0.00269 | 0.00442 | 0.00269 | 0.00269 |
| Max | 652 | 1850 | 652 | 1850 | 652 | 1850 |
| n (Samp) | 383 | 92 | 451 | 20 | 297 | 79 |
| n (Patient) | 383 | 92 | 451 | 20 | 297 | 79 |

| | At Enrollment | | |
|---|---|---|---|
| | sCr or UO | sCr only | UO only |
| AUC | 0.69 | 0.74 | 0.69 |
| SE | 0.033 | 0.065 | 0.036 |
| p | 3.5E−9 | 2.0E−4 | 1.8E−7 |
| nCohort 1 | 383 | 451 | 297 |
| nCohort 2 | 92 | 20 | 79 |
| Cutoff 1 | 0.236 | 2.65 | 0.236 |
| Sens 1 | 71% | 70% | 71% |
| Spec 1 | 53% | 76% | 52% |
| Cutoff 2 | 0.0151 | 0.244 | 0.0151 |
| Sens 2 | 82% | 80% | 81% |
| Spec 2 | 40% | 50% | 37% |
| Cutoff 3 | 0.0109 | 0.0160 | 0.0109 |
| Sens 3 | 90% | 90% | 91% |
| Spec 3 | 33% | 39% | 28% |
| Cutoff 4 | 1.19 | 1.65 | 1.14 |
| Sens 4 | 58% | 75% | 57% |
| Spec 4 | 70% | 70% | 70% |
| Cutoff 5 | 3.20 | 4.17 | 3.20 |
| Sens 5 | 43% | 60% | 43% |
| Spec 5 | 80% | 80% | 80% |
| Cutoff 6 | 9.95 | 15.0 | 12.8 |
| Sens 6 | 30% | 40% | 28% |
| Spec 6 | 90% | 90% | 90% |
| OR Quart 2 | 2.4 | 1.5 | 2.2 |
| p Value | 0.035 | 0.66 | 0.086 |
| 95% CI of | 1.1 | 0.25 | 0.89 |
| OR Quart 2 | 5.6 | 9.1 | 5.4 |
| OR Quart 3 | 2.7 | 0.99 | 3.1 |
| p Value | 0.016 | 0.99 | 0.011 |
| 95% CI of | 1.2 | 0.14 | 1.3 |
| OR Quart 3 | 6.3 | 7.2 | 7.4 |
| OR Quart 4 | 6.4 | 7.1 | 6.1 |
| p Value | 3.1E−6 | 0.011 | 2.4E−5 |
| 95% CI of | 2.9 | 1.6 | 2.6 |
| OR Quart 4 | 14 | 32 | 14 |

Insulin-like growth factor-binding protein 3

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 339 | 706 | 370 | 1120 | 364 | 793 |
| Average | 575 | 1170 | 652 | 1490 | 607 | 1230 |
| Stdev | 735 | 1640 | 939 | 1810 | 762 | 1740 |
| p (t-test) | | 7.8E−8 | | 1.7E−4 | | 1.4E−6 |
| Min | 3.84 | 0.311 | 0.311 | 44.1 | 3.84 | 0.311 |
| Max | 7920 | 12500 | 12500 | 8160 | 7920 | 12500 |
| n (Samp) | 412 | 96 | 482 | 21 | 325 | 83 |
| n (Patient) | 412 | 96 | 482 | 21 | 325 | 83 |

| | At Enrollment | | |
|---|---|---|---|
| | sCr or UO | sCr only | UO only |
| AUC | 0.68 | 0.68 | 0.66 |
| SE | 0.032 | 0.066 | 0.035 |
| p | 5.3E−8 | 0.0050 | 3.1E−6 |
| nCohort 1 | 412 | 482 | 325 |
| nCohort 2 | 96 | 21 | 83 |
| Cutoff 1 | 371 | 425 | 369 |
| Sens 1 | 71% | 71% | 71% |
| Spec 1 | 54% | 54% | 50% |
| Cutoff 2 | 293 | 269 | 293 |
| Sens 2 | 80% | 81% | 81% |
| Spec 2 | 45% | 40% | 42% |
| Cutoff 3 | 114 | 108 | 125 |
| Sens 3 | 91% | 90% | 90% |
| Spec 3 | 26% | 23% | 23% |
| Cutoff 4 | 667 | 736 | 703 |
| Sens 4 | 52% | 52% | 52% |
| Spec 4 | 70% | 70% | 70% |
| Cutoff 5 | 1010 | 1070 | 1030 |
| Sens 5 | 41% | 52% | 40% |
| Spec 5 | 80% | 80% | 80% |
| Cutoff 6 | 1350 | 1470 | 1370 |
| Sens 6 | 28% | 38% | 29% |
| Spec 6 | 90% | 90% | 90% |
| OR Quart 2 | 1.7 | 0.99 | 2.1 |
| p Value | 0.17 | 0.99 | 0.075 |
| 95% CI of | 0.79 | 0.20 | 0.93 |
| OR Quart 2 | 3.9 | 5.0 | 4.8 |
| OR Quart 3 | 2.8 | 1.3 | 2.2 |
| p Value | 0.0063 | 0.71 | 0.052 |
| 95% CI of | 1.3 | 0.29 | 0.99 |
| OR Quart 3 | 6.0 | 6.1 | 5.1 |
| OR Quart 4 | 4.8 | 3.9 | 4.6 |
| p Value | 1.9E−5 | 0.041 | 1.1E−4 |
| 95% CI of | 2.4 | 1.1 | 2.1 |
| OR Quart 4 | 10.0 | 14 | 10.0 |

Immunoglogulin G1

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 3190 | 7960 | 3630 | 14900 | 3200 | 7950 |
| Average | 5790 | 11100 | 6510 | 14000 | 5780 | 11500 |
| Stdev | 7390 | 11900 | 8530 | 10300 | 7370 | 12400 |
| p (t-test) | | 9.1E−8 | | 1.7E−4 | | 2.8E−7 |
| Min | 59.7 | 55.9 | 55.9 | 177 | 59.7 | 55.9 |
| Max | 80000 | 80000 | 80000 | 28800 | 80000 | 80000 |
| n (Samp) | 379 | 92 | 447 | 20 | 292 | 79 |
| n (Patient) | 379 | 92 | 447 | 20 | 292 | 79 |

At Enrollment

| | sCr or UO | sCr only | UO only |
|---|---|---|---|
| AUC | 0.66 | 0.70 | 0.67 |
| SE | 0.033 | 0.066 | 0.036 |
| p | 9.5E−7 | 0.0021 | 1.4E−6 |
| nCohort 1 | 379 | 447 | 292 |
| nCohort 2 | 92 | 20 | 79 |
| Cutoff 1 | 3420 | 7950 | 3630 |
| Sens 1 | 71% | 70% | 71% |
| Spec 1 | 53% | 75% | 53% |
| Cutoff 2 | 1900 | 2280 | 2320 |
| Sens 2 | 80% | 80% | 81% |
| Spec 2 | 28% | 34% | 36% |
| Cutoff 3 | 1420 | 1250 | 1440 |
| Sens 3 | 90% | 90% | 91% |
| Spec 3 | 21% | 16% | 20% |
| Cutoff 4 | 6150 | 6670 | 6150 |
| Sens 4 | 57% | 70% | 57% |
| Spec 4 | 70% | 70% | 70% |
| Cutoff 5 | 8420 | 9330 | 8540 |
| Sens 5 | 47% | 60% | 46% |
| Spec 5 | 80% | 80% | 80% |
| Cutoff 6 | 13900 | 16200 | 13400 |
| Sens 6 | 32% | 40% | 35% |
| Spec 6 | 90% | 90% | 90% |
| OR Quart 2 | 0.85 | 0.99 | 0.75 |
| p Value | 0.68 | 0.99 | 0.50 |
| 95% CI of | 0.39 | 0.20 | 0.32 |
| OR Quart 2 | 1.8 | 5.0 | 1.7 |
| OR Quart 3 | 1.2 | 0.32 | 1.3 |
| p Value | 0.60 | 0.33 | 0.46 |
| 95% CI of | 0.59 | 0.033 | 0.62 |
| OR Quart 3 | 2.5 | 3.2 | 2.9 |
| OR Quart 4 | 3.6 | 4.7 | 3.5 |
| p Value | 9.8E−5 | 0.018 | 4.7E−4 |
| 95% CI of | 1.9 | 1.3 | 1.7 |
| OR Quart 4 | 6.9 | 17 | 7.1 |

Immunoglogulin G2

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 8420 | 18400 | 9310 | 36400 | 9250 | 18300 |
| Average | 17300 | 46300 | 20800 | 69400 | 17000 | 48900 |
| Stdev | 30200 | 64200 | 37700 | 72400 | 28000 | 67700 |
| p (t-test) | | 4.4E−10 | | 1.3E−7 | | 7.9E−10 |
| Min | 119 | 25.4 | 25.4 | 2380 | 334 | 25.4 |
| Max | 240000 | 240000 | 240000 | 203000 | 240000 | 240000 |
| n (Samp) | 379 | 92 | 447 | 20 | 292 | 79 |
| n (Patient) | 379 | 92 | 447 | 20 | 292 | 79 |

| | At Enrollment | | |
|---|---|---|---|
| | sCr or UO | sCr only | UO only |
| AUC | 0.69 | 0.72 | 0.69 |
| SE | 0.033 | 0.066 | 0.036 |
| p | 2.1E−8 | 8.8E−4 | 1.4E−7 |
| nCohort 1 | 379 | 447 | 292 |
| nCohort 2 | 92 | 20 | 79 |
| Cutoff 1 | 8630 | 13500 | 8840 |
| Sens 1 | 71% | 70% | 71% |
| Spec 1 | 52% | 64% | 50% |
| Cutoff 2 | 7050 | 7680 | 7950 |
| Sens 2 | 80% | 80% | 81% |
| Spec 2 | 44% | 43% | 46% |
| Cutoff 3 | 3980 | 4240 | 3980 |
| Sens 3 | 90% | 90% | 91% |
| Spec 3 | 21% | 21% | 20% |
| Cutoff 4 | 14400 | 15900 | 14500 |
| Sens 4 | 57% | 65% | 57% |
| Spec 4 | 70% | 70% | 70% |
| Cutoff 5 | 19800 | 22200 | 19800 |
| Sens 5 | 45% | 55% | 46% |
| Spec 5 | 80% | 80% | 80% |
| Cutoff 6 | 35300 | 41300 | 31100 |
| Sens 6 | 30% | 45% | 33% |
| Spec 6 | 90% | 90% | 90% |
| OR Quart 2 | 1.5 | 0.66 | 1.5 |
| p Value | 0.34 | 0.65 | 0.40 |
| 95% CI of | 0.67 | 0.11 | 0.61 |
| OR Quart 2 | 3.2 | 4.0 | 3.5 |
| OR Quart 3 | 2.0 | 1.3 | 2.1 |
| p Value | 0.071 | 0.71 | 0.078 |
| 95% CI of | 0.94 | 0.29 | 0.92 |
| OR Quart 3 | 4.3 | 6.1 | 4.8 |
| OR Quart 4 | 4.7 | 3.9 | 5.2 |
| p Value | 2.0E−5 | 0.040 | 3.4E−5 |
| 95% CI of | 2.3 | 1.1 | 2.4 |
| OR Quart 4 | 9.5 | 14 | 11 |

Interleukin-11

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 122 | 203 | 129 | 258 | 139 | 196 |
| Average | 194 | 364 | 217 | 438 | 209 | 354 |
| Stdev | 247 | 441 | 288 | 499 | 260 | 424 |
| p (t-test) | | 9.3E−7 | | 0.0013 | | 1.9E−4 |
| Min | 0.154 | 28.3 | 0.154 | 41.6 | 2.83 | 28.3 |
| Max | 2260 | 2140 | 2260 | 2020 | 2260 | 2140 |
| n (Samp) | 385 | 91 | 452 | 20 | 298 | 78 |
| n (Patient) | 385 | 91 | 452 | 20 | 298 | 78 |

| | At Enrollment | | |
|---|---|---|---|
| | sCr or UO | sCr only | UO only |
| AUC | 0.66 | 0.71 | 0.63 |
| SE | 0.034 | 0.066 | 0.037 |
| p | 1.2E−6 | 0.0019 | 5.6E−4 |
| nCohort 1 | 385 | 452 | 298 |
| nCohort 2 | 91 | 20 | 78 |
| Cutoff 1 | 117 | 201 | 113 |
| Sens 1 | 70% | 70% | 71% |
| Spec 1 | 49% | 70% | 42% |
| Cutoff 2 | 94.5 | 179 | 94.5 |
| Sens 2 | 80% | 80% | 81% |
| Spec 2 | 42% | 65% | 35% |
| Cutoff 3 | 63.9 | 56.7 | 64.5 |
| Sens 3 | 90% | 90% | 91% |
| Spec 3 | 29% | 23% | 24% |
| Cutoff 4 | 185 | 203 | 203 |
| Sens 4 | 54% | 65% | 47% |
| Spec 4 | 70% | 70% | 70% |
| Cutoff 5 | 253 | 286 | 274 |
| Sens 5 | 41% | 40% | 38% |
| Spec 5 | 80% | 80% | 80% |
| Cutoff 6 | 416 | 479 | 436 |
| Sens 6 | 22% | 25% | 22% |
| Spec 6 | 90% | 90% | 90% |
| OR Quart 2 | 2.9 | 0.33 | 2.7 |
| p Value | 0.010 | 0.34 | 0.015 |
| 95% CI of | 1.3 | 0.034 | 1.2 |
| OR Quart 2 | 6.6 | 3.2 | 6.1 |
| OR Quart 3 | 2.6 | 1.7 | 1.5 |
| p Value | 0.022 | 0.48 | 0.38 |
| 95% CI of | 1.1 | 0.40 | 0.62 |
| OR Quart 3 | 6.0 | 7.3 | 3.5 |
| OR Quart 4 | 5.7 | 3.9 | 4.1 |
| p Value | 1.2E−5 | 0.039 | 3.9E−4 |
| 95% CI of | 2.6 | 1.1 | 1.9 |
| OR Quart 4 | 13 | 15 | 9.1 |

Interleukin-2 receptor alpha chain

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 518 | 937 | 566 | 1560 | 524 | 913 |
| Average | 922 | 1840 | 1040 | 2330 | 943 | 1910 |
| Stdev | 1120 | 2190 | 1310 | 2770 | 1100 | 2260 |
| p (t-test) | | 2.1E−8 | | 6.2E−5 | | 1.6E−7 |
| Min | 0.0339 | 0.0482 | 0.0339 | 0.0482 | 0.0339 | 0.115 |
| Max | 8370 | 10100 | 9660 | 10100 | 6540 | 10100 |
| n (Samp) | 385 | 92 | 453 | 20 | 298 | 79 |
| n (Patient) | 385 | 92 | 453 | 20 | 298 | 79 |

| | At Enrollment | | |
|---|---|---|---|
| | sCr or UO | sCr only | UO only |
| AUC | 0.64 | 0.61 | 0.65 |
| SE | 0.034 | 0.068 | 0.037 |
| p | 3.4E−5 | 0.096 | 7.0E−5 |
| nCohort 1 | 385 | 453 | 298 |
| nCohort 2 | 92 | 20 | 79 |
| Cutoff 1 | 467 | 385 | 484 |
| Sens 1 | 71% | 70% | 71% |
| Spec 1 | 48% | 41% | 48% |
| Cutoff 2 | 325 | 60.5 | 333 |
| Sens 2 | 80% | 80% | 81% |
| Spec 2 | 41% | 13% | 39% |
| Cutoff 3 | 91.3 | 0.120 | 152 |
| Sens 3 | 90% | 90% | 91% |
| Spec 3 | 17% | 2% | 23% |
| Cutoff 4 | 1090 | 1170 | 1110 |
| Sens 4 | 42% | 60% | 41% |
| Spec 4 | 70% | 70% | 70% |
| Cutoff 5 | 1600 | 1690 | 1660 |
| Sens 5 | 36% | 50% | 33% |
| Spec 5 | 80% | 80% | 80% |
| Cutoff 6 | 2280 | 2590 | 2390 |
| Sens 6 | 26% | 30% | 27% |
| Spec 6 | 90% | 90% | 90% |
| OR Quart 2 | 1.6 | 0.39 | 2.4 |
| p Value | 0.24 | 0.27 | 0.058 |
| 95% CI of | 0.73 | 0.074 | 0.97 |
| OR Quart 2 | 3.5 | 2.0 | 5.8 |
| OR Quart 3 | 2.9 | 0.59 | 4.1 |
| p Value | 0.0045 | 0.48 | 0.0012 |
| 95% CI of | 1.4 | 0.14 | 1.7 |
| OR Quart 3 | 6.0 | 2.5 | 9.7 |
| OR Quart 4 | 3.4 | 2.1 | 4.5 |
| p Value | 8.9E−4 | 0.20 | 5.2E−4 |
| 95% CI of | 1.6 | 0.69 | 1.9 |
| OR Quart 4 | 6.9 | 6.3 | 10 |

Neutrophil collagenase

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 3190 | 9770 | 3750 | 21900 | 3260 | 10400 |
| Average | 13100 | 49700 | 16000 | 113000 | 11100 | 46700 |
| Stdev | 44400 | 112000 | 52900 | 169000 | 27800 | 99400 |
| p (t-test) | | 4.0E−7 | | 5.7E−12 | | 2.6E−8 |
| Min | 0.114 | 25.8 | 0.114 | 25.8 | 0.114 | 198 |
| Max | 670000 | 670000 | 670000 | 649000 | 300000 | 670000 |
| n (Samp) | 412 | 96 | 482 | 21 | 326 | 83 |
| n (Patient) | 412 | 96 | 482 | 21 | 326 | 83 |

| | At Enrollment | | |
|---|---|---|---|
| | sCr or UO | sCr only | UO only |
| AUC | 0.71 | 0.72 | 0.72 |
| SE | 0.032 | 0.064 | 0.034 |
| p | 1.1E−10 | 7.6E−4 | 5.9E−11 |
| nCohort 1 | 412 | 482 | 326 |
| nCohort 2 | 96 | 21 | 83 |
| Cutoff 1 | 4980 | 8140 | 5380 |
| Sens 1 | 71% | 71% | 71% |
| Spec 1 | 60% | 68% | 63% |
| Cutoff 2 | 2810 | 2810 | 3170 |
| Sens 2 | 80% | 81% | 81% |
| Spec 2 | 48% | 44% | 49% |
| Cutoff 3 | 1020 | 191 | 1630 |
| Sens 3 | 91% | 90% | 90% |
| Spec 3 | 27% | 5% | 35% |
| Cutoff 4 | 7060 | 8960 | 6920 |
| Sens 4 | 66% | 67% | 66% |
| Spec 4 | 70% | 70% | 70% |
| Cutoff 5 | 11900 | 15600 | 10700 |
| Sens 5 | 46% | 62% | 49% |
| Spec 5 | 80% | 80% | 80% |
| Cutoff 6 | 22200 | 30800 | 21900 |
| Sens 6 | 34% | 48% | 36% |
| Spec 6 | 90% | 90% | 90% |
| OR Quart 2 | 1.7 | 0.24 | 3.0 |
| p Value | 0.22 | 0.21 | 0.030 |
| 95% CI of | 0.73 | 0.027 | 1.1 |
| OR Quart 2 | 3.9 | 2.2 | 8.0 |
| OR Quart 3 | 3.0 | 0.74 | 4.4 |
| p Value | 0.0054 | 0.69 | 0.0022 |
| 95% CI of | 1.4 | 0.16 | 1.7 |
| OR Quart 3 | 6.5 | 3.4 | 11 |
| OR Quart 4 | 6.2 | 3.5 | 9.8 |
| p Value | 1.4E−6 | 0.033 | 1.1E−6 |
| 95% CI of | 3.0 | 1.1 | 3.9 |
| OR Quart 4 | 13 | 11 | 24 |

Protransforming growth factor alpha

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 6.28 | 12.5 | 6.98 | 15.7 | 6.62 | 12.3 |
| Average | 11.2 | 17.0 | 11.7 | 28.1 | 12.3 | 14.7 |
| Stdev | 24.0 | 18.0 | 22.5 | 32.1 | 26.8 | 10.2 |
| p (t-test) | | 0.030 | | 0.0019 | | 0.45 |
| Min | 0.00262 | 0.00454 | 0.00262 | 1.41 | 0.00262 | 0.00454 |
| Max | 361 | 122 | 361 | 122 | 361 | 50.4 |
| n (Samp) | 385 | 92 | 453 | 20 | 298 | 79 |
| n (Patient) | 385 | 92 | 453 | 20 | 298 | 79 |

| | At Enrollment | | |
|---|---|---|---|
| | sCr or UO | sCr only | UO only |
| AUC | 0.71 | 0.74 | 0.69 |
| SE | 0.033 | 0.065 | 0.036 |
| p | 3.0E−10 | 2.9E−4 | 1.5E−7 |
| nCohort 1 | 385 | 453 | 298 |
| nCohort 2 | 92 | 20 | 79 |
| Cutoff 1 | 8.32 | 11.3 | 8.57 |
| Sens 1 | 71% | 70% | 71% |
| Spec 1 | 62% | 69% | 61% |
| Cutoff 2 | 6.15 | 6.78 | 6.19 |
| Sens 2 | 80% | 80% | 81% |
| Spec 2 | 49% | 49% | 47% |
| Cutoff 3 | 4.83 | 4.92 | 4.83 |
| Sens 3 | 90% | 90% | 91% |
| Spec 3 | 42% | 37% | 39% |
| Cutoff 4 | 10.4 | 11.6 | 10.8 |
| Sens 4 | 61% | 65% | 59% |
| Spec 4 | 70% | 70% | 70% |
| Cutoff 5 | 13.8 | 15.2 | 14.7 |
| Sens 5 | 41% | 60% | 37% |
| Spec 5 | 80% | 80% | 80% |
| Cutoff 6 | 21.9 | 23.8 | 24.9 |
| Sens 6 | 20% | 35% | 10% |
| Spec 6 | 90% | 90% | 90% |
| OR Quart 2 | 2.1 | 4.1 | 2.0 |
| p Value | 0.12 | 0.21 | 0.16 |
| 95% CI of | 0.83 | 0.45 | 0.76 |
| OR Quart 2 | 5.5 | 37 | 5.2 |
| OR Quart 3 | 5.9 | 2.0 | 5.3 |
| p Value | 5.9E−5 | 0.57 | 2.4E−4 |
| 95% CI of | 2.5 | 0.18 | 2.2 |
| OR Quart 3 | 14 | 23 | 13 |
| OR Quart 4 | 7.7 | 14 | 6.0 |
| p Value | 2.8E−6 | 0.011 | 6.6E−5 |
| 95% CI of | 3.3 | 1.8 | 2.5 |
| OR Quart 4 | 18 | 110 | 15 |

CA 15-3

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 434 | 434 | nd | nd | 434 | 434 |
| Average | 300 | 382 | nd | nd | 303 | 391 |
| Stdev | 250 | 216 | nd | nd | 251 | 213 |
| p (t-test) | | 0.070 | nd | nd | | 0.059 |
| Min | 2.21 | 4.72 | nd | nd | 2.21 | 4.72 |
| Max | 784 | 784 | nd | nd | 784 | 784 |
| n (Samp) | 169 | 36 | nd | nd | 150 | 34 |
| n (Patient) | 169 | 36 | nd | nd | 150 | 34 |

| | At Enrollment | | |
|---|---|---|---|
| | sCr or UO | sCr only | UO only |
| AUC | 0.62 | nd | 0.62 |
| SE | 0.054 | nd | 0.056 |
| p | 0.033 | nd | 0.028 |
| nCohort 1 | 169 | nd | 150 |
| nCohort 2 | 36 | nd | 34 |
| Cutoff 1 | 427 | nd | 427 |
| Sens 1 | 75% | nd | 76% |
| Spec 1 | 49% | nd | 48% |
| Cutoff 2 | 80.1 | nd | 80.1 |
| Sens 2 | 81% | nd | 82% |
| Spec 2 | 36% | nd | 37% |
| Cutoff 3 | 36.1 | nd | 51.3 |
| Sens 3 | 92% | nd | 91% |
| Spec 3 | 24% | nd | 31% |
| Cutoff 4 | 434 | nd | 434 |
| Sens 4 | 14% | nd | 15% |
| Spec 4 | 88% | nd | 88% |
| Cutoff 5 | 434 | nd | 434 |
| Sens 5 | 14% | nd | 15% |
| Spec 5 | 88% | nd | 88% |
| Cutoff 6 | 784 | nd | 784 |
| Sens 6 | 0% | nd | 0% |
| Spec 6 | 100% | nd | 100% |
| OR Quart 2 | 1.3 | nd | 2.1 |
| p Value | 0.73 | nd | 0.30 |
| 95% CI of | 0.32 | nd | 0.50 |
| OR Quart 2 | 5.1 | nd | 9.2 |
| OR Quart 3 | 8.9 | nd | 11 |
| p Value | 2.2E−4 | nd | 3.2E−4 |
| 95% CI of | 2.8 | nd | 3.0 |
| OR Quart 3 | 28 | nd | 41 |
| OR Quart 4 | 1.2 | nd | 1.7 |
| p Value | 0.75 | nd | 0.46 |
| 95% CI of | 0.32 | nd | 0.39 |
| OR Quart 4 | 4.9 | nd | 7.8 |

TABLE 12
Comparison of marker levels in enroll EDTA samples collected
from Cohort 1 (patients that did not progress beyond RIFLE stage
0 or R within 48 hrs) and in enroll EDTA samples collected from
Cohort 2 (subjects reaching RIFLE stage I or F within 48 hrs).
Enroll samples from patients already at stage I or F were included in
Cohort 2.
C-C motif chemokine 18

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 171 | 222 | nd | nd | 160 | 241 |
| Average | 238 | 264 | nd | nd | 230 | 269 |
| Stdev | 192 | 149 | nd | nd | 180 | 151 |
| p (t-test) | | 0.51 | nd | nd | | 0.32 |
| Min | 48.2 | 103 | nd | nd | 48.2 | 103 |
| Max | 1020 | 682 | nd | nd | 817 | 682 |
| n (Samp) | 109 | 28 | nd | nd | 98 | 25 |
| n (Patient) | 109 | 28 | nd | nd | 98 | 25 |

| | At Enrollment | | |
|---|---|---|---|
| | sCr or UO | sCr only | UO only |
| AUC | 0.61 | nd | 0.63 |
| SE | 0.062 | nd | 0.065 |
| p | 0.078 | nd | 0.047 |
| nCohort 1 | 109 | nd | 98 |
| nCohort 2 | 28 | nd | 25 |
| Cutoff 1 | 157 | nd | 158 |
| Sens 1 | 71% | nd | 72% |
| Spec 1 | 48% | nd | 50% |
| Cutoff 2 | 150 | nd | 151 |
| Sens 2 | 82% | nd | 80% |
| Spec 2 | 45% | nd | 48% |
| Cutoff 3 | 117 | nd | 117 |
| Sens 3 | 93% | nd | 92% |
| Spec 3 | 31% | nd | 31% |
| Cutoff 4 | 264 | nd | 260 |
| Sens 4 | 39% | nd | 44% |
| Spec 4 | 71% | nd | 70% |
| Cutoff 5 | 340 | nd | 321 |
| Sens 5 | 25% | nd | 24% |
| Spec 5 | 81% | nd | 81% |
| Cutoff 6 | 548 | nd | 548 |
| Sens 6 | 4% | nd | 4% |
| Spec 6 | 91% | nd | 91% |
| OR Quart 2 | 4.9 | nd | 3.4 |
| p Value | 0.056 | nd | 0.16 |
| 95% CI of | 0.96 | nd | 0.62 |
| OR Quart 2 | 25 | nd | 18 |
| OR Quart 3 | 5.8 | nd | 5.7 |
| p Value | 0.034 | nd | 0.036 |
| 95% CI of | 1.1 | nd | 1.1 |
| OR Quart 3 | 29 | nd | 29 |
| OR Quart 4 | 5.5 | nd | 4.9 |
| p Value | 0.038 | nd | 0.059 |
| 95% CI of | 1.1 | nd | 0.94 |
| OR Quart 4 | 28 | nd | 25 |

C-C motif chemokine 24

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 268 | 224 | 257 | 509 | 275 | 179 |
| Average | 445 | 370 | 419 | 569 | 468 | 291 |
| Stdev | 477 | 370 | 455 | 465 | 495 | 298 |
| p (t-test) | | 0.46 | | 0.40 | | 0.11 |
| Min | 0.0260 | 12.4 | 0.0260 | 68.0 | 13.1 | 12.4 |
| Max | 2920 | 1290 | 2920 | 1290 | 2920 | 1100 |
| n (Samp) | 89 | 26 | 108 | 7 | 81 | 22 |
| n (Patient) | 89 | 26 | 108 | 7 | 81 | 22 |

| | At Enrollment | | |
|---|---|---|---|
| | sCr or UO | sCr only | UO only |
| AUC | 0.44 | 0.62 | 0.37 |
| SE | 0.065 | 0.12 | 0.070 |
| p | 0.40 | 0.29 | 0.066 |
| nCohort 1 | 89 | 108 | 81 |
| nCohort 2 | 26 | 7 | 22 |
| Cutoff 1 | 94.1 | 279 | 86.2 |
| Sens 1 | 73% | 71% | 73% |
| Spec 1 | 17% | 56% | 14% |
| Cutoff 2 | 67.2 | 169 | 39.1 |
| Sens 2 | 81% | 86% | 82% |
| Spec 2 | 9% | 36% | 4% |
| Cutoff 3 | 25.7 | 67.2 | 25.7 |
| Sens 3 | 92% | 100% | 91% |
| Spec 3 | 3% | 12% | 2% |
| Cutoff 4 | 511 | 490 | 542 |
| Sens 4 | 27% | 57% | 14% |
| Spec 4 | 71% | 70% | 70% |
| Cutoff 5 | 737 | 668 | 761 |
| Sens 5 | 15% | 29% | 9% |
| Spec 5 | 81% | 81% | 80% |
| Cutoff 6 | 1110 | 1080 | 1110 |
| Sens 6 | 4% | 29% | 0% |
| Spec 6 | 91% | 91% | 90% |
| OR Quart 2 | 1.5 | 0.96 | 1.8 |
| p Value | 0.52 | 0.98 | 0.45 |
| 95% CI of | 0.42 | 0.057 | 0.39 |
| OR Quart 2 | 5.5 | 16 | 8.6 |
| OR Quart 3 | 1.0 | 3.1 | 1.8 |
| p Value | 1.0 | 0.34 | 0.45 |
| 95% CI of | 0.26 | 0.30 | 0.39 |
| OR Quart 3 | 3.9 | 32 | 8.6 |
| OR Quart 4 | 2.3 | 2.0 | 4.3 |
| p Value | 0.20 | 0.58 | 0.049 |
| 95% CI of | 0.65 | 0.17 | 1.0 |
| OR Quart 4 | 7.9 | 23 | 18 |

C-C motif chemokine 8

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 12.8 | 15.4 | 14.0 | 14.4 | 12.8 | 15.7 |
| Average | 21.9 | 51.7 | 21.2 | 143 | 22.4 | 57.6 |
| Stdev | 31.4 | 144 | 29.0 | 270 | 32.9 | 156 |
| p (t-test) | | 0.070 | | 1.3E−5 | | 0.060 |
| Min | 0.162 | 6.24 | 0.162 | 12.4 | 0.162 | 6.24 |
| Max | 177 | 740 | 177 | 740 | 177 | 740 |
| n (Samp) | 89 | 26 | 108 | 7 | 81 | 22 |
| n (Patient) | 89 | 26 | 108 | 7 | 81 | 22 |

| | At Enrollment | | |
|---|---|---|---|
| | sCr or UO | sCr only | UO only |
| AUC | 0.60 | 0.68 | 0.61 |
| SE | 0.065 | 0.11 | 0.070 |
| p | 0.13 | 0.11 | 0.11 |
| nCohort 1 | 89 | 108 | 81 |
| nCohort 2 | 26 | 7 | 22 |
| Cutoff 1 | 13.0 | 13.7 | 13.7 |
| Sens 1 | 73% | 71% | 73% |
| Spec 1 | 51% | 47% | 54% |
| Cutoff 2 | 12.4 | 13.0 | 12.3 |
| Sens 2 | 81% | 86% | 82% |
| Spec 2 | 45% | 47% | 47% |
| Cutoff 3 | 9.32 | 12.3 | 9.32 |
| Sens 3 | 92% | 100% | 91% |
| Spec 3 | 27% | 41% | 28% |
| Cutoff 4 | 19.9 | 18.9 | 18.9 |
| Sens 4 | 27% | 43% | 27% |
| Spec 4 | 71% | 70% | 70% |
| Cutoff 5 | 22.4 | 22.4 | 22.4 |
| Sens 5 | 27% | 43% | 27% |
| Spec 5 | 81% | 81% | 80% |
| Cutoff 6 | 34.9 | 33.4 | 34.9 |
| Sens 6 | 15% | 43% | 14% |
| Spec 6 | 91% | 91% | 90% |
| OR Quart 2 | 2.7 | >3.2 | 2.7 |
| p Value | 0.19 | <0.32 | 0.26 |
| 95% CI of | 0.61 | >0.32 | 0.48 |
| OR Quart 2 | 12 | na | 16 |
| OR Quart 3 | 3.8 | >1.0 | 6.1 |
| p Value | 0.071 | <1.0 | 0.032 |
| 95% CI of | 0.89 | >0.060 | 1.2 |
| OR Quart 3 | 16 | na | 32 |
| OR Quart 4 | 2.7 | >3.2 | 3.4 |
| p Value | 0.19 | <0.32 | 0.16 |
| 95% CI of | 0.61 | >0.32 | 0.62 |
| OR Quart 4 | 12 | na | 19 |

Cathepsin D

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 234000 | 230000 | nd | nd | 240000 | 244000 |
| Average | 266000 | 284000 | nd | nd | 278000 | 279000 |
| Stdev | 146000 | 165000 | nd | nd | 153000 | 154000 |
| p (t-test) | | 0.55 | nd | nd | | 0.99 |
| Min | 34100 | 126000 | nd | nd | 54000 | 126000 |
| Max | 802000 | 655000 | nd | nd | 802000 | 618000 |
| n (Samp) | 109 | 28 | nd | nd | 98 | 25 |
| n (Patient) | 109 | 28 | nd | nd | 98 | 25 |

| | At Enrollment | | |
|---|---|---|---|
| | sCr or UO | sCr only | UO only |
| AUC | 0.53 | nd | 0.50 |
| SE | 0.062 | nd | 0.065 |
| p | 0.66 | nd | 1.0 |
| nCohort 1 | 109 | nd | 98 |
| nCohort 2 | 28 | nd | 25 |
| Cutoff 1 | 170000 | nd | 170000 |
| Sens 1 | 71% | nd | 72% |
| Spec 1 | 36% | nd | 34% |
| Cutoff 2 | 144000 | nd | 144000 |
| Sens 2 | 82% | nd | 80% |
| Spec 2 | 21% | nd | 18% |
| Cutoff 3 | 135000 | nd | 130000 |
| Sens 3 | 93% | nd | 92% |
| Spec 3 | 17% | nd | 14% |
| Cutoff 4 | 324000 | nd | 333000 |
| Sens 4 | 29% | nd | 28% |
| Spec 4 | 71% | nd | 70% |
| Cutoff 5 | 375000 | nd | 382000 |
| Sens 5 | 25% | nd | 24% |
| Spec 5 | 81% | nd | 81% |
| Cutoff 6 | 471000 | nd | 489000 |
| Sens 6 | 18% | nd | 16% |
| Spec 6 | 91% | nd | 91% |
| OR Quart 2 | 1.4 | nd | 0.82 |
| p Value | 0.55 | nd | 0.76 |
| 95% CI of | 0.44 | nd | 0.24 |
| OR Quart 2 | 4.7 | nd | 2.8 |
| OR Quart 3 | 1.0 | nd | 0.82 |
| p Value | 1.0 | nd | 0.76 |
| 95% CI of | 0.29 | nd | 0.24 |
| OR Quart 3 | 3.5 | nd | 2.8 |
| OR Quart 4 | 1.4 | nd | 0.86 |
| p Value | 0.59 | nd | 0.81 |
| 95% CI of | 0.42 | nd | 0.25 |
| OR Quart 4 | 4.5 | nd | 2.9 |

C—X—C motif chemokine 13

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 55.0 | 53.1 | 53.1 | 54.5 | 55.6 | 53.1 |
| Average | 155 | 212 | 139 | 612 | 164 | 239 |
| Stdev | 292 | 530 | 268 | 949 | 304 | 573 |
| p (t-test) | | 0.48 | | 5.5E−4 | | 0.41 |
| Min | 9.90 | 15.1 | 9.90 | 15.1 | 9.90 | 15.6 |
| Max | 1790 | 2000 | 1790 | 2000 | 1790 | 2000 |
| n (Samp) | 89 | 26 | 108 | 7 | 81 | 22 |
| n (Patient) | 89 | 26 | 108 | 7 | 81 | 22 |

| | At Enrollment | | |
|---|---|---|---|
| | sCr or UO | sCr only | UO only |
| AUC | 0.47 | 0.57 | 0.48 |
| SE | 0.065 | 0.12 | 0.070 |
| p | 0.67 | 0.55 | 0.76 |
| nCohort 1 | 89 | 108 | 81 |
| nCohort 2 | 26 | 7 | 22 |
| Cutoff 1 | 34.2 | 35.0 | 34.2 |
| Sens 1 | 73% | 71% | 73% |
| Spec 1 | 33% | 32% | 32% |
| Cutoff 2 | 28.5 | 34.2 | 28.5 |
| Sens 2 | 81% | 86% | 82% |
| Spec 2 | 25% | 32% | 23% |
| Cutoff 3 | 15.6 | 14.8 | 18.1 |
| Sens 3 | 92% | 100% | 91% |
| Spec 3 | 6% | 4% | 12% |
| Cutoff 4 | 114 | 101 | 116 |
| Sens 4 | 19% | 43% | 18% |
| Spec 4 | 71% | 70% | 70% |
| Cutoff 5 | 194 | 157 | 194 |
| Sens 5 | 12% | 29% | 14% |
| Spec 5 | 81% | 81% | 80% |
| Cutoff 6 | 271 | 267 | 271 |
| Sens 6 | 12% | 29% | 14% |
| Spec 6 | 91% | 91% | 90% |
| OR Quart 2 | 1.8 | 2.0 | 1.6 |
| p Value | 0.35 | 0.58 | 0.48 |
| 95% CI of | 0.52 | 0.17 | 0.41 |
| OR Quart 2 | 6.5 | 23 | 6.7 |
| OR Quart 3 | 1.5 | 0.96 | 2.0 |
| p Value | 0.52 | 0.98 | 0.31 |
| 95% CI of | 0.42 | 0.057 | 0.51 |
| OR Quart 3 | 5.5 | 16 | 8.0 |
| OR Quart 4 | 1.3 | 3.1 | 1.4 |
| p Value | 0.69 | 0.34 | 0.67 |
| 95% CI of | 0.35 | 0.30 | 0.32 |
| OR Quart 4 | 4.9 | 32 | 5.8 |

Insulin-like growth factor-binding protein 3

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 3010 | 3200 | nd | nd | 2940 | 2720 |
| Average | 3450 | 3420 | nd | nd | 3380 | 3240 |
| Stdev | 1530 | 1520 | nd | nd | 1500 | 1470 |
| p (t-test) | | 0.93 | nd | nd | | 0.67 |
| Min | 730 | 1120 | nd | nd | 730 | 1120 |
| Max | 7370 | 7100 | nd | nd | 7260 | 7100 |
| n (Samp) | 109 | 28 | nd | nd | 98 | 25 |
| n (Patient) | 109 | 28 | nd | nd | 98 | 25 |

| | At Enrollment | | |
|---|---|---|---|
| | sCr or UO | sCr only | UO only |
| AUC | 0.50 | nd | 0.48 |
| SE | 0.061 | nd | 0.065 |
| p | 0.97 | nd | 0.70 |
| nCohort 1 | 109 | nd | 98 |
| nCohort 2 | 28 | nd | 25 |
| Cutoff 1 | 2410 | nd | 2370 |
| Sens 1 | 71% | nd | 72% |
| Spec 1 | 32% | nd | 32% |
| Cutoff 2 | 2050 | nd | 2050 |
| Sens 2 | 82% | nd | 80% |
| Spec 2 | 19% | nd | 20% |
| Cutoff 3 | 1400 | nd | 1400 |
| Sens 3 | 93% | nd | 92% |
| Spec 3 | 6% | nd | 6% |
| Cutoff 4 | 4410 | nd | 4350 |
| Sens 4 | 29% | nd | 24% |
| Spec 4 | 71% | nd | 70% |
| Cutoff 5 | 4970 | nd | 4900 |
| Sens 5 | 14% | nd | 12% |
| Spec 5 | 81% | nd | 81% |
| Cutoff 6 | 5550 | nd | 5520 |
| Sens 6 | 7% | nd | 4% |
| Spec 6 | 91% | nd | 91% |
| OR Quart 2 | 1.2 | nd | 1.5 |
| p Value | 0.72 | nd | 0.52 |
| 95% CI of | 0.39 | nd | 0.42 |
| OR Quart 2 | 3.9 | nd | 5.4 |
| OR Quart 3 | 0.86 | nd | 1.2 |
| p Value | 0.80 | nd | 0.74 |
| 95% CI of | 0.26 | nd | 0.34 |
| OR Quart 3 | 2.9 | nd | 4.6 |
| OR Quart 4 | 1.0 | nd | 1.6 |
| p Value | 0.95 | nd | 0.48 |
| 95% CI of | 0.32 | nd | 0.44 |
| OR Quart 4 | 3.4 | nd | 5.7 |

Immunoglogulin G1

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 8690000 | 1.04E7 | nd | nd | 8030000 | 1.04E7 |
| Average | 1.02E7 | 1.14E7 | nd | nd | 9810000 | 1.14E7 |
| Stdev | 5690000 | 5580000 | nd | nd | 5570000 | 5580000 |
| p (t-test) | | 0.44 | nd | nd | | 0.30 |
| Min | 3270000 | 3270000 | nd | nd | 3270000 | 3270000 |
| Max | 3.57E7 | 2.63E7 | nd | nd | 3.57E7 | 2.63E7 |
| n (Samp) | 81 | 17 | nd | nd | 74 | 17 |
| n (Patient) | 81 | 17 | nd | nd | 74 | 17 |

| | At Enrollment | | |
|---|---|---|---|
| | sCr or UO | sCr only | UO only |
| AUC | 0.59 | nd | 0.61 |
| SE | 0.079 | nd | 0.079 |
| p | 0.24 | nd | 0.15 |
| nCohort 1 | 81 | nd | 74 |
| nCohort 2 | 17 | nd | 17 |
| Cutoff 1 | 9280000 | nd | 9280000 |
| Sens 1 | 71% | nd | 71% |
| Spec 1 | 56% | nd | 59% |
| Cutoff 2 | 7600000 | nd | 7600000 |
| Sens 2 | 82% | nd | 82% |
| Spec 2 | 43% | nd | 46% |
| Cutoff 3 | 4390000 | nd | 4390000 |
| Sens 3 | 94% | nd | 94% |
| Spec 3 | 6% | nd | 7% |
| Cutoff 4 | 1.11E7 | nd | 1.09E7 |
| Sens 4 | 41% | nd | 47% |
| Spec 4 | 70% | nd | 70% |
| Cutoff 5 | 1.34E7 | nd | 1.32E7 |
| Sens 5 | 18% | nd | 18% |
| Spec 5 | 80% | nd | 81% |
| Cutoff 6 | 1.65E7 | nd | 1.52E7 |
| Sens 6 | 18% | nd | 18% |
| Spec 6 | 90% | nd | 91% |
| OR Quart 2 | 1.5 | nd | 1.5 |
| p Value | 0.67 | nd | 0.67 |
| 95% CI of | 0.23 | nd | 0.23 |
| OR Quart 2 | 9.9 | nd | 10.0 |
| OR Quart 3 | 6.6 | nd | 5.3 |
| p Value | 0.026 | nd | 0.052 |
| 95% CI of | 1.2 | nd | 0.99 |
| OR Quart 3 | 35 | nd | 29 |
| OR Quart 4 | 1.5 | nd | 2.1 |
| p Value | 0.67 | nd | 0.42 |
| 95% CI of | 0.23 | nd | 0.34 |
| OR Quart 4 | 9.9 | nd | 13 |

Immunoglogulin G2

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 1.08E7 | 1.08E7 | nd | nd | 1.03E7 | 1.08E7 |
| Average | 1.38E7 | 1.11E7 | nd | nd | 1.32E7 | 1.11E7 |
| Stdev | 9680000 | 6680000 | nd | nd | 8830000 | 6680000 |
| p (t-test) | | 0.28 | nd | nd | | 0.37 |
| Min | 563000 | 2450000 | nd | nd | 1750000 | 2450000 |
| Max | 4.88E7 | 2.70E7 | nd | nd | 4.10E7 | 2.70E7 |
| n (Samp) | 81 | 17 | nd | nd | 74 | 17 |
| n (Patient) | 81 | 17 | nd | nd | 74 | 17 |

| | At Enrollment | | |
|---|---|---|---|
| | sCr or UO | sCr only | UO only |
| AUC | 0.44 | nd | 0.46 |
| SE | 0.079 | nd | 0.079 |
| p | 0.45 | nd | 0.58 |
| nCohort 1 | 81 | nd | 74 |
| nCohort 2 | 17 | nd | 17 |
| Cutoff 1 | 7850000 | nd | 7850000 |
| Sens 1 | 71% | nd | 71% |
| Spec 1 | 28% | nd | 30% |
| Cutoff 2 | 1750000 | nd | 1750000 |
| Sens 2 | 100% | nd | 100% |
| Spec 2 | 7% | nd | 7% |
| Cutoff 3 | 1750000 | nd | 1750000 |
| Sens 3 | 100% | nd | 100% |
| Spec 3 | 7% | nd | 7% |
| Cutoff 4 | 1.64E7 | nd | 1.64E7 |
| Sens 4 | 12% | nd | 12% |
| Spec 4 | 70% | nd | 73% |
| Cutoff 5 | 2.01E7 | nd | 1.84E7 |
| Sens 5 | 6% | nd | 6% |
| Spec 5 | 80% | nd | 81% |
| Cutoff 6 | 2.66E7 | nd | 2.55E7 |
| Sens 6 | 6% | nd | 6% |
| Spec 6 | 90% | nd | 91% |
| OR Quart 2 | 4.7 | nd | 4.6 |
| p Value | 0.072 | nd | 0.079 |
| 95% CI of | 0.87 | nd | 0.84 |
| OR Quart 2 | 26 | nd | 25 |
| OR Quart 3 | 1.6 | nd | 2.2 |
| p Value | 0.64 | nd | 0.39 |
| 95% CI of | 0.24 | nd | 0.36 |
| OR Quart 3 | 10 | nd | 13 |
| OR Quart 4 | 3.0 | nd | 2.3 |
| p Value | 0.21 | nd | 0.36 |
| 95% CI of | 0.53 | nd | 0.38 |
| OR Quart 4 | 17 | nd | 14 |

Interleukin-11

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 61.5 | 89.1 | 62.2 | 111 | 61.5 | 97.6 |
| Average | 97.6 | 146 | 95.1 | 316 | 98.2 | 142 |
| Stdev | 111 | 226 | 105 | 390 | 113 | 225 |
| p (t-test) | | 0.14 | | 5.8E−5 | | 0.20 |
| Min | 0.359 | 0.480 | 0.359 | 0.920 | 0.359 | 0.480 |
| Max | 741 | 1060 | 741 | 1060 | 741 | 1060 |
| n (Samp) | 90 | 26 | 109 | 7 | 82 | 22 |
| n (Patient) | 90 | 26 | 109 | 7 | 82 | 22 |

| | At Enrollment | | |
|---|---|---|---|
| | sCr or UO | sCr only | UO only |
| AUC | 0.53 | 0.64 | 0.54 |
| SE | 0.065 | 0.12 | 0.070 |
| p | 0.63 | 0.22 | 0.54 |
| nCohort 1 | 90 | 109 | 82 |
| nCohort 2 | 26 | 7 | 22 |
| Cutoff 1 | 20.4 | 47.4 | 33.9 |
| Sens 1 | 73% | 71% | 73% |
| Spec 1 | 27% | 46% | 32% |
| Cutoff 2 | 15.1 | 20.4 | 15.1 |
| Sens 2 | 81% | 86% | 82% |
| Spec 2 | 21% | 28% | 21% |
| Cutoff 3 | 0.736 | 0.736 | 6.00 |
| Sens 3 | 92% | 100% | 91% |
| Spec 3 | 11% | 11% | 15% |
| Cutoff 4 | 132 | 132 | 132 |
| Sens 4 | 31% | 43% | 32% |
| Spec 4 | 70% | 71% | 71% |
| Cutoff 5 | 173 | 171 | 177 |
| Sens 5 | 19% | 43% | 18% |
| Spec 5 | 80% | 81% | 80% |
| Cutoff 6 | 209 | 209 | 209 |
| Sens 6 | 15% | 43% | 14% |
| Spec 6 | 90% | 91% | 90% |
| OR Quart 2 | 1.0 | 2.1 | 0.76 |
| p Value | 1.0 | 0.56 | 0.71 |
| 95% CI of | 0.28 | 0.18 | 0.18 |
| OR Quart 2 | 3.6 | 24 | 3.2 |
| OR Quart 3 | 1.2 | 1.0 | 1.5 |
| p Value | 0.75 | 1.0 | 0.51 |
| 95% CI of | 0.35 | 0.060 | 0.42 |
| OR Quart 3 | 4.2 | 17 | 5.7 |
| OR Quart 4 | 1.2 | 3.2 | 1.3 |
| p Value | 0.75 | 0.32 | 0.73 |
| 95% CI of | 0.35 | 0.32 | 0.33 |
| OR Quart 4 | 4.2 | 33 | 4.8 |

Interleukin-2 receptor alpha chain

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 25.5 | 27.4 | 25.4 | 33.6 | 28.1 | 27.4 |
| Average | 69.7 | 82.6 | 70.8 | 102 | 74.9 | 93.3 |
| Stdev | 141 | 146 | 139 | 193 | 147 | 157 |
| p (t-test) | | 0.68 | | 0.58 | | 0.61 |
| Min | 0.0290 | 0.0461 | 0.0290 | 0.0569 | 0.0290 | 0.0461 |
| Max | 918 | 562 | 918 | 533 | 918 | 562 |
| n (Samp) | 90 | 26 | 109 | 7 | 82 | 22 |
| n (Patient) | 90 | 26 | 109 | 7 | 82 | 22 |

| | At Enrollment | | |
|---|---|---|---|
| | sCr or UO | sCr only | UO only |
| AUC | 0.53 | 0.51 | 0.52 |
| SE | 0.065 | 0.11 | 0.070 |
| p | 0.69 | 0.90 | 0.73 |
| nCohort 1 | 90 | 109 | 82 |
| nCohort 2 | 26 | 7 | 22 |
| Cutoff 1 | 5.04 | 4.75 | 10.6 |
| Sens 1 | 73% | 71% | 73% |
| Spec 1 | 30% | 28% | 32% |
| Cutoff 2 | 0.0515 | 0.0515 | 0.0515 |
| Sens 2 | 88% | 100% | 86% |
| Spec 2 | 16% | 16% | 15% |
| Cutoff 3 | 0.0359 | 0.0515 | 0.0359 |
| Sens 3 | 100% | 100% | 100% |
| Spec 3 | 9% | 16% | 7% |
| Cutoff 4 | 50.7 | 60.8 | 52.5 |
| Sens 4 | 42% | 29% | 45% |
| Spec 4 | 70% | 71% | 71% |
| Cutoff 5 | 93.9 | 94.9 | 104 |
| Sens 5 | 19% | 14% | 18% |
| Spec 5 | 80% | 81% | 80% |
| Cutoff 6 | 153 | 155 | 155 |
| Sens 6 | 15% | 14% | 14% |
| Spec 6 | 90% | 91% | 90% |
| OR Quart 2 | 1.2 | 0.48 | 1.3 |
| p Value | 0.75 | 0.56 | 0.73 |
| 95% CI of | 0.35 | 0.041 | 0.33 |
| OR Quart 2 | 4.2 | 5.6 | 4.8 |
| OR Quart 3 | 1.0 | 1.0 | 1.0 |
| p Value | 1.0 | 1.0 | 1.0 |
| 95% CI of | 0.28 | 0.13 | 0.25 |
| OR Quart 3 | 3.6 | 7.6 | 4.0 |
| OR Quart 4 | 1.2 | 1.0 | 1.3 |
| p Value | 0.75 | 1.0 | 0.73 |
| 95% CI of | 0.35 | 0.13 | 0.33 |
| OR Quart 4 | 4.2 | 7.6 | 4.8 |

Neutrophil collagenase

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 1260 | 1780 | nd | nd | 1190 | 2410 |
| Average | 3150 | 8240 | nd | nd | 3130 | 9130 |
| Stdev | 5690 | 17400 | nd | nd | 5890 | 18200 |
| p (t-test) | | 0.011 | nd | nd | | 0.0067 |
| Min | 83.9 | 1.14 | nd | nd | 1.14 | 241 |
| Max | 44900 | 83900 | nd | nd | 44900 | 83900 |
| n (Samp) | 108 | 28 | nd | nd | 97 | 25 |
| n (Patient) | 108 | 28 | nd | nd | 97 | 25 |

| | At Enrollment | | |
|---|---|---|---|
| | sCr or UO | sCr only | UO only |
| AUC | 0.58 | nd | 0.63 |
| SE | 0.062 | nd | 0.065 |
| p | 0.19 | nd | 0.041 |
| nCohort 1 | 108 | nd | 97 |
| nCohort 2 | 28 | nd | 25 |
| Cutoff 1 | 1040 | nd | 1130 |
| Sens 1 | 71% | nd | 72% |
| Spec 1 | 41% | nd | 49% |
| Cutoff 2 | 810 | nd | 994 |
| Sens 2 | 82% | nd | 80% |
| Spec 2 | 29% | nd | 41% |
| Cutoff 3 | 261 | nd | 712 |
| Sens 3 | 93% | nd | 92% |
| Spec 3 | 6% | nd | 30% |
| Cutoff 4 | 2370 | nd | 2340 |
| Sens 4 | 46% | nd | 52% |
| Spec 4 | 70% | nd | 70% |
| Cutoff 5 | 3750 | nd | 3580 |
| Sens 5 | 29% | nd | 32% |
| Spec 5 | 81% | nd | 80% |
| Cutoff 6 | 7620 | nd | 7370 |
| Sens 6 | 21% | nd | 24% |
| Spec 6 | 91% | nd | 91% |
| OR Quart 2 | 2.7 | nd | 5.7 |
| p Value | 0.13 | nd | 0.036 |
| 95% CI of | 0.74 | nd | 1.1 |
| OR Quart 2 | 9.8 | nd | 29 |
| OR Quart 3 | 1.6 | nd | 2.8 |
| p Value | 0.50 | nd | 0.24 |
| 95% CI of | 0.41 | nd | 0.50 |
| OR Quart 3 | 6.3 | nd | 16 |
| OR Quart 4 | 2.7 | nd | 5.7 |
| p Value | 0.13 | nd | 0.036 |
| 95% CI of | 0.74 | nd | 1.1 |
| OR Quart 4 | 9.8 | nd | 29 |

Protransforming growth factor alpha

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 1.47 | 4.03 | 1.70 | 7.33 | 1.40 | 4.03 |
| Average | 8.43 | 12.3 | 8.77 | 17.3 | 8.98 | 12.7 |
| Stdev | 32.1 | 18.1 | 30.0 | 22.4 | 33.6 | 19.0 |
| p (t-test) | | 0.56 | | 0.46 | | 0.62 |
| Min | 0.00228 | 0.00228 | 0.00228 | 0.227 | 0.00228 | 0.00228 |
| Max | 245 | 68.3 | 245 | 62.6 | 245 | 68.3 |
| n (Samp) | 90 | 26 | 109 | 7 | 82 | 22 |
| n (Patient) | 90 | 26 | 109 | 7 | 82 | 22 |

| | At Enrollment | | |
|---|---|---|---|
| | sCr or UO | sCr only | UO only |
| AUC | 0.68 | 0.76 | 0.69 |
| SE | 0.063 | 0.11 | 0.068 |
| p | 0.0044 | 0.018 | 0.0058 |
| nCohort 1 | 90 | 109 | 82 |
| nCohort 2 | 26 | 7 | 22 |
| Cutoff 1 | 1.78 | 5.22 | 1.78 |
| Sens 1 | 73% | 71% | 73% |
| Spec 1 | 56% | 83% | 59% |
| Cutoff 2 | 0.885 | 2.57 | 0.885 |
| Sens 2 | 81% | 86% | 82% |
| Spec 2 | 39% | 59% | 41% |
| Cutoff 3 | 0.141 | 0.141 | 0.783 |
| Sens 3 | 92% | 100% | 91% |
| Spec 3 | 21% | 19% | 39% |
| Cutoff 4 | 2.99 | 3.27 | 2.93 |
| Sens 4 | 50% | 71% | 50% |
| Spec 4 | 70% | 71% | 71% |
| Cutoff 5 | 4.00 | 4.91 | 4.23 |
| Sens 5 | 50% | 71% | 50% |
| Spec 5 | 80% | 81% | 80% |
| Cutoff 6 | 5.64 | 13.6 | 6.65 |
| Sens 6 | 42% | 29% | 41% |
| Spec 6 | 90% | 91% | 90% |
| OR Quart 2 | 1.8 | 0 | 2.2 |
| p Value | 0.45 | na | 0.39 |
| 95% CI of | 0.39 | na | 0.36 |
| OR Quart 2 | 8.4 | na | 13 |
| OR Quart 3 | 1.8 | 1.0 | 2.9 |
| p Value | 0.45 | 1.0 | 0.24 |
| 95% CI of | 0.39 | 0.060 | 0.50 |
| OR Quart 3 | 8.4 | 17 | 16 |
| OR Quart 4 | 7.0 | 5.8 | 8.8 |
| p Value | 0.0063 | 0.12 | 0.0093 |
| 95% CI of | 1.7 | 0.64 | 1.7 |
| OR Quart 4 | 29 | 53 | 45 |

CA 15-3

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 0.309 | 0.529 | nd | nd | 0.324 | 0.506 |
| Average | 1.36 | 0.742 | nd | nd | 1.48 | 0.757 |
| Stdev | 5.87 | 0.707 | nd | nd | 6.18 | 0.748 |
| p (t-test) | | 0.58 | nd | nd | | 0.56 |
| Min | 0.0328 | 0.109 | nd | nd | 0.0328 | 0.109 |
| Max | 60.0 | 3.12 | nd | nd | 60.0 | 3.12 |
| n (Samp) | 109 | 28 | nd | nd | 98 | 25 |
| n (Patient) | 109 | 28 | nd | nd | 98 | 25 |

| | At Enrollment | | |
|---|---|---|---|
| | sCr or UO | sCr only | UO only |
| AUC | 0.65 | nd | 0.62 |
| SE | 0.061 | nd | 0.066 |
| p | 0.017 | nd | 0.071 |
| nCohort 1 | 109 | nd | 98 |
| nCohort 2 | 28 | nd | 25 |
| Cutoff 1 | 0.392 | nd | 0.385 |
| Sens 1 | 71% | nd | 72% |
| Spec 1 | 59% | nd | 55% |
| Cutoff 2 | 0.208 | nd | 0.208 |
| Sens 2 | 86% | nd | 84% |
| Spec 2 | 35% | nd | 34% |
| Cutoff 3 | 0.181 | nd | 0.181 |
| Sens 3 | 93% | nd | 92% |
| Spec 3 | 32% | nd | 31% |
| Cutoff 4 | 0.510 | nd | 0.535 |
| Sens 4 | 54% | nd | 44% |
| Spec 4 | 71% | nd | 70% |
| Cutoff 5 | 0.762 | nd | 0.864 |
| Sens 5 | 29% | nd | 24% |
| Spec 5 | 81% | nd | 81% |
| Cutoff 6 | 1.91 | nd | 2.03 |
| Sens 6 | 7% | nd | 8% |
| Spec 6 | 91% | nd | 91% |
| OR Quart 2 | 2.8 | nd | 2.7 |
| p Value | 0.25 | nd | 0.26 |
| 95% CI of | 0.50 | nd | 0.48 |
| OR Quart 2 | 15 | nd | 15 |
| OR Quart 3 | 8.7 | nd | 6.7 |
| p Value | 0.0077 | nd | 0.022 |
| 95% CI of | 1.8 | nd | 1.3 |
| OR Quart 3 | 43 | nd | 34 |
| OR Quart 4 | 5.5 | nd | 4.9 |
| p Value | 0.038 | nd | 0.059 |
| 95% CI of | 1.1 | nd | 0.94 |
| OR Quart 4 | 28 | nd | 25 |

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method for evaluating renal status in a subject, comprising:
obtaining a urine sample from a subject selected for evaluation based on a determination that the subject is at risk of a future or current acute renal injury;
performing one or more assays configured to detect one or more biomarkers selected from the group consisting of Cancer antigen CA 15-3, C-C Motif chemokine 18, C-C Motif chemokine 24, Cathepsin D, C-X-C Motif chemokine 13, C-C motif chemokine 8, Interleukin-2 receptor alpha chain, Insulin-like growth factor-binding protein 3, Interleukin-11, Matrix Metalloproteinase-8, Transforming growth factor alpha, IgG1, and IgG2 by introducing the urine sample obtained from the subject into an assay instrument which (i) for each analyte binding assay performed, contacts all or a portion of the urine sample with a binding reagent which specifically binds for detection the kidney injury marker which is assayed, and (ii) generates one or more assay results indicative of binding of each biomarker which is assayed to its respective binding reagent and displays the assay results generated in human readable form; and
correlating the assay result(s) generated by the assay instrument to the renal status of the subject.

2. A method according to claim 1, wherein said correlation step comprises correlating the assay result(s) to one or more of risk stratification, diagnosis, staging, prognosis, classifying and monitoring of the renal status of the subject.

3. A method according to claim 1, wherein the subject is selected for evaluation based on a determination that the subject is at risk of a future acute renal injury.

4. A method according to claim 3, wherein the subject is selected for evaluation based on a determination that the subject is at risk of a future injury to renal function, future reduced renal function, future improvement in renal function, and future acute renal failure (ARF).

5. A method according to claim 1, wherein said assay results comprise at least 2, 3, 4, or 5 of:
a measured concentration of Cancer antigen CA 15-3,
a measured concentration of C-C Motif chemokine 18,
a measured concentration of C-C Motif chemokine 24,
a measured concentration of Cathepsin D,
a measured concentration of C-X-C Motif chemokine 13,
a measured concentration of C-C motif chemokine 8,
a measured concentration of Interleukin-2 receptor alpha chain,
a measured concentration of Insulin-like growth factor-binding protein 3,
a measured concentration of Interleukin-11,
a measured concentration of Matrix Metalloproteinase-8,
a measured concentration of Transforming growth factor alpha,
a measured concentration of IgG1, and
a measured concentration of IgG2.

6. A method according to claim 5, wherein a plurality of assay results are combined using a function that converts the plurality of assay results into a single composite result.

7. (canceled)

8. A method according to claim 3, wherein the subject is selected for evaluation based on a determination that the subject is at risk of a future acute renal injury within 30 days of the time at which the urine sample is obtained from the subject.

9. A method according to claim 8, wherein the subject is selected for evaluation based on a determination that the subject is at risk of a future acute renal injury within a period selected from the group consisting of 21 days, 14 days, 7 days, 5 days, 96 hours, 72 hours, 48 hours, 36 hours, 24 hours, and 12 hours.

10. A method according to claim 1, wherein the subject is selected for evaluation of renal status based on the pre-existence in the subject of one or more known risk factors for prerenal, intrinsic renal, or postrenal ARF.

11. A method according to claim 1, wherein the subject is selected for evaluation of renal status based on an existing diagnosis of one or more of congestive heart failure, preeclampsia, eclampsia, diabetes mellitus, hypertension, coronary artery disease, proteinuria, renal insufficiency, glomerular filtration below the normal range, cirrhosis, serum creatinine above the normal range, sepsis, injury to renal function, reduced renal function, or ARF, or based on undergoing or having undergone major vascular surgery, coronary artery bypass, or other cardiac surgery, or based on exposure to NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin.

12. A method according to claim 1, wherein each assay is an immunoassay performed by (i) introducing the urine sample into an assay device comprising at least one of which binds to a biomarker which is assayed, and (ii) generating an assay result indicative of binding of each biomarker to its respective antibody.

13. A method according to claim 1, wherein said correlating step comprises assessing whether or not renal function is improving or worsening in a subject who has suffered from an injury to renal function, reduced renal function, or ARF based on the assay result(s).

14-23. (canceled)

24. A method according to claim 1, wherein said one or more future changes in renal status comprise one or more of a future injury to renal function, future reduced renal function, future improvement in renal function, and future acute renal failure (ARF) within 72 hours of the time at which the body fluid sample is obtained.

25. A method according to claim 1, wherein said correlating step comprises correlating the assay results to a likelihood of one or more of a future injury to renal function, future reduced renal function, future improvement in renal function, and future acute renal failure (ARF) within 48 hours of the time at which the body fluid sample is obtained.

26. A method according to claim 1, wherein correlating step comprises correlating the assay results to a likelihood of one or more of a future injury to renal function, future reduced renal function, future improvement in renal function, and future acute renal failure (ARF) within 24 hours of the time at which the body fluid sample is obtained.

27. A method according to claim 1, wherein the subject is in RIFLE stage 0 or R.

28. A method according to claim 27, wherein the subject is in RIFLE stage 0.

29-32. (canceled)

33. A method according to claim 27, wherein the subject is in RIFLE stage R.

34. (canceled)

35. A method according to claim 1, wherein the subject is in RIFLE stage 0, R, or I.

36. A method according to claim 35, wherein the subject is in RIFLE stage I.

37-54. (canceled)

55. A method according to claim 1, wherein the subject is not in acute renal failure.

56-108. (canceled)
